(19) United States
(12) Patent Application Publication
(10) Pub. No.: US 2024/0357925 A1
(43) Pub. Date: Oct. 24, 2024

(54) NOVEL COMPOUND AND ORGANIC LIGHT EMITTING DEVICE COMPRISING THE SAME

(71) Applicant: LG CHEM, LTD., Seoul (KR)

(72) Inventors: Min Woo Jung, Daejeon (KR); Dong Hoon Lee, Daejeon (KR); Miyeon Han, Daejeon (KR); Seulchan Park, Daejeon (KR); Hoon Jun Kim, Daejeon (KR); Hye Min Cho, Daejeon (KR); Hojung Lee, Daejeon (KR)

(21) Appl. No.: 18/681,573

(22) PCT Filed: Oct. 26, 2022
(86) PCT No.: PCT/KR2022/016473

§ 371 Date: Feb. 6, 2024

(30) Foreign Application Priority Data
Oct. 27, 2021 (KR) 10-2021-0144838
Oct. 26, 2022 (KR) 10-2022-0138849

(51) Int. Cl. H10K 85/60; C07D 405/14; C07D 409/14; C09K 11/06; H10K 50/12

(52) U.S. Cl. CPC H10K 85/654; C07D 405/14; C07D 409/14; C09K 11/06; H10K 85/6572; H10K 85/6574; H10K 85/6576; C09K 2211/1018; H10K 50/12

(57) ABSTRACT

A novel compound of Chemical Formula 1 and an organic light emitting device including the same:

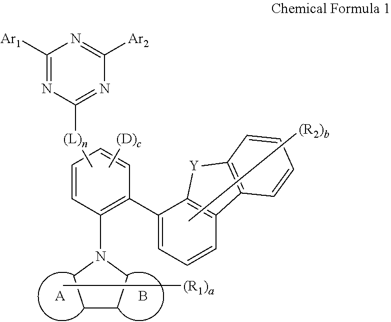

where Y is O or S: Ar₁ and Ar₂ are each independently a substituted or unsubstituted C₆₋₆₀ aryl or C₂₋₆₀ heteroaryl containing one or more heteroatoms of N, O and S; A and B are each independently a C₆₋₆₀ aromatic ring or a C₂₋₆₀ heteroaromatic ring containing one or more heteroatoms of N, O and S which is fused with an adjacent pentagonal ring; D is deuterium; R₁ and R₂ are each independently deuterium or a substituted or unsubstituted C₆₋₆₀ aryl or C₂₋₆₀ heteroaryl containing one or more heteroatoms of N, O and S, provided that at least one of R₁ and R₂ is deuterium; and the other substituent are as defined in the specification.

### CROSS-REFERENCE TO RELATED APPLICATION(S)

This application is a National Stage Application of International Application No. PCT/KR2022/016473 filed on Oct. 26, 2022, which claims priority to and the benefit of Korean Patent Application No. 10-2021-0144838 filed on Oct. 27, 2021 and Korean Patent Application No. 10-2022-0138849 filed on Oct. 26, 2022 in the Korean Intellectual Property Office, the entire contents of which are incorporated herein by reference.

### TECHNICAL FIELD

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### PRIOR ART LITERATURE

### Patent Literature

(Patent Literature 0001) Korean Unexamined Patent Publication No. 10-2000-0051826

### BRIEF DESCRIPTION

### Technical Problem

It is an object of the present disclosure to provide a novel compound and an organic light emitting device comprising the same.

### Technical Solution

According to an aspect of the present disclosure, provided is a compound of Chemical Formula 1:

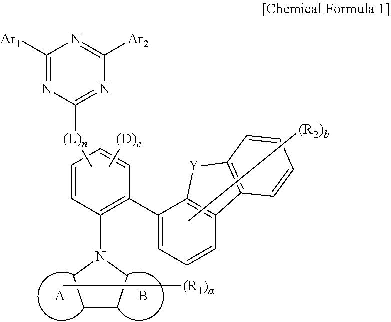

wherein, in Chemical Formula 1:
Y is O or S;
L is a single bond, a substituted or unsubstituted C₆₋₆₀ arylene, or a substituted or unsubstituted C₂₋₆₀ heteroarylene containing one or more heteroatoms of N, O and S;
n is 1, 2, or 3;
Ar₁ and Ar₂ are each independently a substituted or unsubstituted C₆₋₆₀ aryl or a substituted or unsubstituted C₂₋₆₀ heteroaryl containing one or more heteroatoms of N, O and S;
A and B are each independently a C₆₋₆₀ aromatic ring or a C₂₋₆₀ heteroaromatic ring containing one or more heteroatoms of N, O and S which is fused with an adjacent pentagonal ring;
R₁ and R₂ are each independently deuterium, a substituted or unsubstituted C₆₋₆₀ aryl, or a substituted or unsubstituted C₂₋₆₀ heteroaryl containing one or more heteroatoms of N, O and S;
D is deuterium;
a is an integer of 0 to 12;
b is an integer of 0 to 7; and
c is an integer of 0 to 3,
with the proviso that at least one of R₁ and R₂ is deuterium, and a+b is 1 or more.

According to another aspect of the present disclosure, provided is an organic light emitting device comprising: a first electrode; a second electrode that is opposite to the first electrode; and one or more organic material layers that are between the first electrode and the second electrode, wherein one or more layers of the one or more organic material layers comprises the compound of Chemical Formula 1.

### Advantageous Effects

The above-mentioned compound of Chemical Formula 1 can be used as a material of an organic material layer in an organic light emitting device, and can improve the efficiency, achieve low driving voltage and/or improve lifetime characteristics in the organic light emitting device.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, an electron transport layer 8, an electron injection layer 9, and a cathode 4.

### DETAILED DESCRIPTION

Hereinafter, embodiments of the present disclosure will be described in more detail to facilitate understanding of the invention.

### Definition of Terms

As used herein, the notation

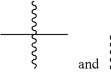

mean a bond linked to another substituent group.

As used herein, the term “substituted or unsubstituted” means being unsubstituted or substituted with one or more substituents selected from the group consisting of deuterium, a halogen group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heteroaryl containing at least one of N, O and S atoms, or being unsubstituted or substituted with a substituent to which two or more substituents of the above-exemplified substituents are linked. For example, “a substituent in which two or more substituents are linked” can be a biphenyl group. Namely, a biphenylyl group can be an aryl group, or it can also be interpreted as a substituent in which two phenyl groups are linked. In one example, the term “substituted or unsubstituted” can be understood to mean “being unsubstituted or substituted with one or more, for example 1 to 5 substituents selected from the group consisting of deuterium, halogen, nitrile, a C₁₋₁₀ alkyl, a C₁₋₁₀ alkoxy and a C₆₋₂₀ aryl”. Further, the term “substituted with one or more substituents” can be understood to mean, for example, “being substituted with 1 to 5 substituents, or “being substituted with 1 to 2 substituents”.

In the present disclosure, the carbon number of a carbonyl group is not particularly limited, but is preferably 1 to 40. Specifically, it can be a substituent having the following structure, but is not limited thereto.

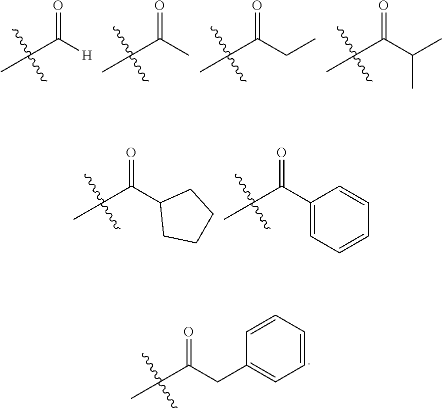

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, it can be a substituent having the following structure, but is not limited thereto:

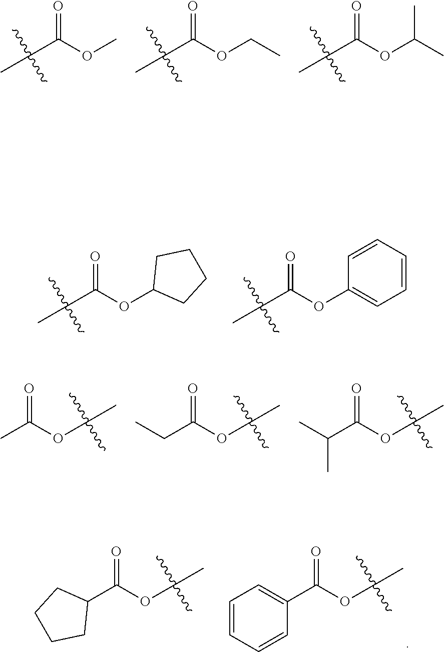

In the present disclosure, the carbon number of an imide group is not particularly limited, but is preferably 1 to 25. Specifically, it can be a substituent having the following structure, but is not limited thereto:

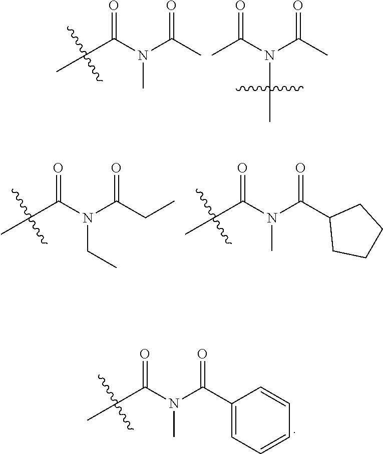

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, specific examples of the alkyl group include methyl, ethyl, propyl, n-propyl, isopropyl, butyl, n-butyl, isobutyl, tert-butyl, sec-butyl, 1-methyl-butyl, 1-ethylbutyl, pentyl, n-pentyl, isopentyl, neopentyl, tert-pentyl, 1-ethyl-propyl, 1,1-dimethylpropyl, hexyl, n-hexyl, 1-methylpentyl, 2-methylpentyl, 4-methyl-2-pentyl, 3,3-dimethylbutyl, 2-ethylbutyl, heptyl, n-heptyl, isohexyl, 1-methylhexyl, 2-methylhexyl, 3-methylhexyl, 4-methylhexyl, 5-methylhexyl, cyclopentylmethyl, cyclohexylmethyl, octyl, n-octyl, tert-octyl, 1-methylheptyl, 2-ethylhexyl, 2,4,4-trimethyl-1-pentyl, 2,4,4-trimethyl-2-pentyl, 2-propylpentyl, n-nonyl, 2,2-dimethylheptyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl) vinyl-1-yl, 2,2-bis(diphenyl-1-yl) vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group having aromaticity. According to one embodiment, the carbon number of the aryl group is 6 to 30. According to one embodiment, the carbon number of the aryl group is 6 to 20. The aryl group can be a phenyl group, a biphenylyl group, a terphenylyl group or the like as the monocyclic aryl group, but is not limited thereto. The polycyclic aryl group includes a naphthyl group, an anthracenyl group, a phenanthrenyl group, a triphenylenyl, pyrenyl group, a perylenyl group, a chrysenyl group, and the like, but is not limited thereto.

In the present disclosure, a heteroaryl is a heteroaryl containing one or more heteroatoms of O, N, Si and S as a heteroatom, and the carbon number thereof is not particularly limited, but is preferably 2 to 60. Examples of the heteroaryl group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, a carbazolyl group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, the arylamine group and the arylsilyl group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine group can be applied to the aforementioned description of the heteroaryl. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heteroaryl can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heteroaryl can be applied, except that the heterocycle is not a monovalent group but formed by combining two substituent groups.

### (Compound)

Meanwhile, the present disclosure provides the compound of Chemical Formula 1.

Specifically, the compound of Chemical Formula 1 relates to a compound in which a carbazole-based substituent, a dibenzofuranyl/dibenzothiophenyl substituent, and a triazinyl substituent binds to a benzene ring, wherein the dibenzofuranyl/dibenzothiophenyl substituent binds to the ortho position of the benzene ring, and the carbazole-based substituent and the dibenzofuranyl/dibenzothiophenyl substituent have a structure in which at least one deuterium substituent is present.

The compound of Chemical Formula 1 can exhibit enhanced electronic stability as a carbazole-based substituent, a dibenzofuranyl/dibenzothiophenyl substituent, and a triazinyl substituent form a bond at a specific position. In addition, the compound can be used as a host material for the light emitting layer. In this case, an exciplex is formed in a radical anion state, wherein due to deuterium (D) contained in the carbazole-based substituent and/or the dibenzofuranyl/dibenzothiophenyl substituent, the vibrational energy of the radical anion state can be lowered to have stable energy, so that the formed exciplex can also become in a more stable state.

Therefore, as compared to compounds containing no deuterium (D) in the same structure; and compounds containing deuterium but not containing deuterium in the carbazole-based substituent and the dibenzofuranyl/dibenzothiophenyl substituent, the compound of Chemical Formula 1 can contribute to the formation of a stable exciplex while having improved thermal stability and electrochemical stability, so that energy transfer to the dopant can be effectively achieved. This makes it possible to particularly improve lifetime characteristics of the organic light emitting device including the compound of Chemical Formula 1.

In addition, the phrase “at least one of R₁ and R₂ is deuterium, and a+b is 1 or more” used herein means that a is 1 or more and at least one of R₁ is deuterium; or b is 1 or more and at least one of R₂ is deuterium.

Also, L can be a single bond, a C₆₋₂₀ arylene that is unsubstituted or substituted with deuterium, or a C₂₋₂₀ heteroarylene containing one heteroatom of O and S that is unsubstituted or substituted with deuterium.

Specifically, L can be a single bond; phenylene that is unsubstituted or substituted with 1 to 4 deuteriums; biphenyldiyl that is unsubstituted or substituted with 1 to 8 deuteriums; terphenyldiyl that is unsubstituted or substituted with 1 to 12 deuteriums; naphthylene that is unsubstituted or substituted with 1 to 6 deuterium; anthracenylene that is unsubstituted or substituted with 1 to 8 deuteriums; dibenzofuranylene that is unsubstituted or substituted with 1 to 6 deuteriums; or dibenzothiophenylene that is unsubstituted or substituted with 1 to 6 deuteriums.

Specifically, L can be a single bond, phenylene, biphenyldiyl, terphenyldiyl, naphthylene, anthracenylene, dibenzofuranylene, or dibenzothiophenylene.

For example, L can be a single bond or any one selected from the group consisting of:

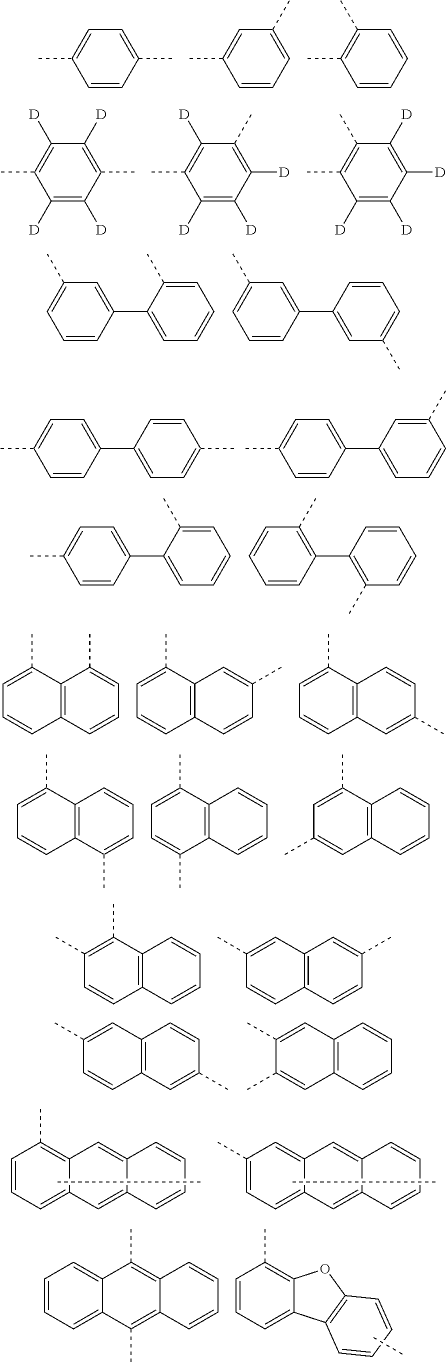

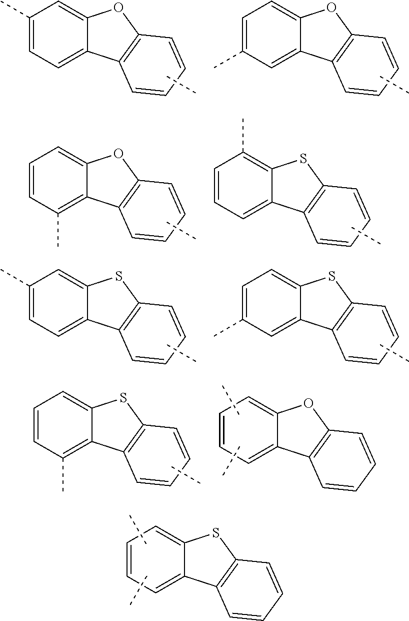

Here, n means the number of L, and when n is 2 or more, 2 or more Ls are the same or different from each other.

Further, —(L)n—can be a single bond or any one selected from the group consisting of:

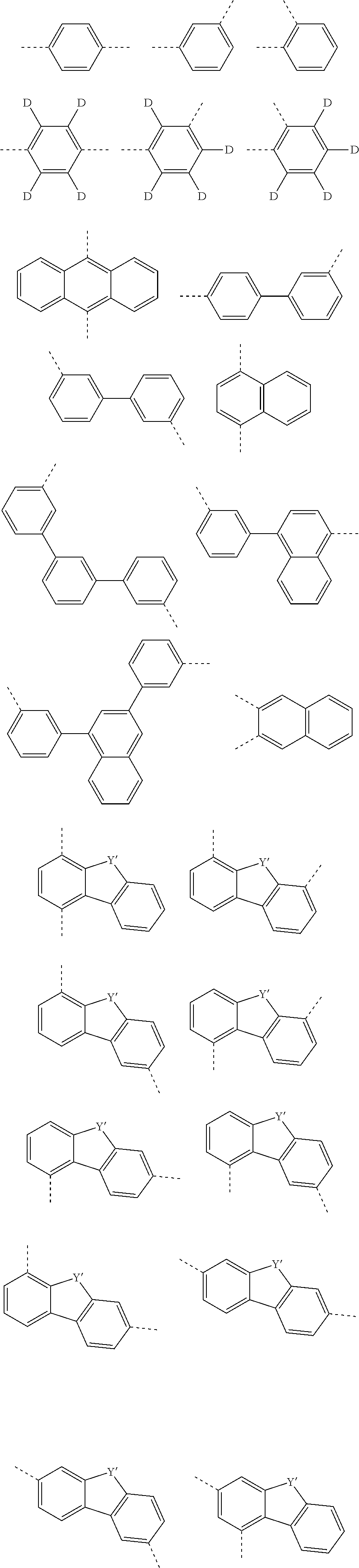

wherein,
Y′ is O or S.

In addition, Ar₁ and Ar₂ can be each independently a C₆₋₂₀ aryl which is unsubstituted or substituted with one or more substituents selected from the group consisting of deuterium and phenyl; or a C₂₋₂₀ heteroaryl containing two heteroatoms of N, O and S which is unsubstituted or substituted with one or more substituents selected from the group consisting of deuterium and phenyl.

Specifically, Ar₁ and Ar₂ are each independently phenyl, biphenylyl, naphthyl, phenanthryl, dibenzofuranyl, dibenzothiophenyl, carbazolyl, or phenylcarbazolyl,
wherein Ar₁ and Ar₂ can be unsubstituted or substituted with at least one deuterium.

More specifically, Ar₁ and Ar₂ can be each independently
phenyl that is unsubstituted or substituted with 1 to 5 deuteriums;
biphenylyl that is unsubstituted or substituted with 1 to 9 deuteriums;
naphthyl that is unsubstituted or substituted with 1 to 7 deuteriums;
phenanthryl that is unsubstituted or substituted with 1 to 9 deuteriums;
dibenzofuranyl that is unsubstituted or substituted with 1 to 7 deuteriums;
dibenzothiophenyl that is unsubstituted or substituted with 1 to 7 deuteriums;
carbazol-9-yl that is unsubstituted or substituted with 1 to 8 deuteriums; or
phenylcarbazol-9-yl that is unsubstituted or substituted with 1 to 12 deuterium atoms.

For example, Ar₁ and Ar₂ can be each independently any one substituent selected from the group consisting of:

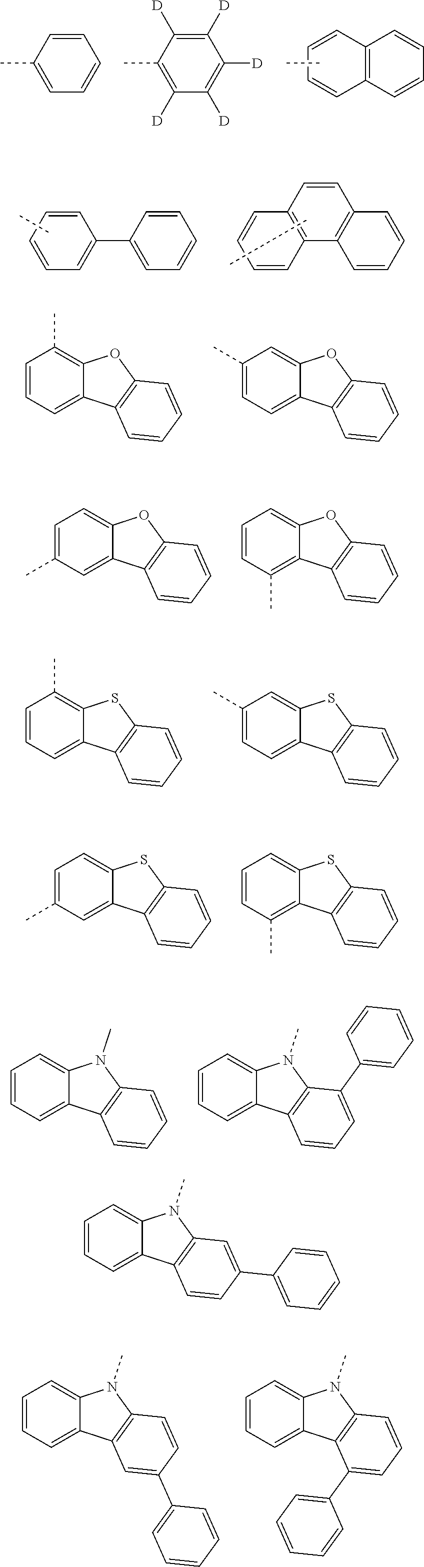

wherein, at least one of Ar₁ and Ar₂ can be

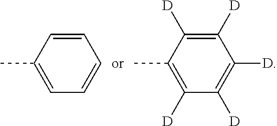

Further, Ar₁ and Ar₂ are the same as or different from each other.

Further, A and B can be each independently a benzene ring, a naphthalene ring, a phenanthrene ring, a triphenylenyl ring, a pyrene ring, an indole ring, or a carbazole ring.

Specifically, A and B can be each independently a benzene ring, a naphthalene ring, a phenanthrene ring, or a carbazole ring.

More specifically, one of A and B can be a benzene ring, and the other is a benzene ring, a naphthalene ring, a phenanthrene ring, or a carbazole ring.

Further, R₁ and R₂ are each independently deuterium or a C₆₋₂₀ aryl that is unsubstituted or substituted with at least one deuterium.

Specifically, R₁ and R₂ can be each independently deuterium or phenyl that is unsubstituted or substituted with 1 to 5 deuteriums.

Further, a is an integer of 1 to 12, and b is 0; or
a is 0, and b is an integer of 1 to 7; or
a is an integer of 1 to 12, and b can be an integer of 1 to 7.

In addition,
a is an integer of 1 to 12, b is 0, and R₁ is deuterium or a C₆₋₂₀ aryl that is unsubstituted or substituted with one or more deuterium, with the proviso that one of R₁ is deuterium; or
a is 0, b is an integer of 1 to 7, and R₂ is deuterium or a C₆₋₂₀ aryl that is unsubstituted or substituted with at least one deuterium, with the proviso that one of R₂ is deuterium; or
a is an integer of 1 to 12, b is an integer of 1 to 7, and R₁ and R₂ are each independently deuterium or a C₆₋₂₀ aryl that is unsubstituted or substituted with at least one deuterium, with the proviso that one of R₁ and R₂ is deuterium.

In one embodiment, a+b can be an integer of 1 to 20.

In another embodiment, a+b can be an integer of 2 to 20.

In another embodiment, a+b can be an integer of 3 to 20.

In another embodiment, a+b can be an integer of 4 to 20.

In another embodiment, a+b can be an integer of 5 to 20.

In another embodiment, a+b can be an integer of 6 to 20.

In another embodiment, a+b can be an integer of 7 to 20.

In another embodiment, a+b can be an integer of 8 to 20.

At this time, when both A and B are benzene rings,
a+b can be 1 or more, 2 or more, 3 or more, 4 or more, 5 or more, 6 or more, 7 or more, 8 or more, 9 or more, or 10 or more, and 15 or less, 14 or less, 13 or less, 12 or less, or 11 or less.

Further, c is 0, 1, 2, or 3.

In one embodiment, a+b+c can be an integer of 1 to 22.

In another embodiment, a+b+c can be an integer from 2 to 22.

In another embodiment, a+b+c can be an integer of 3 to 22.

In another embodiment, a+b+c can be an integer of 4 to 22.

In another embodiment, a+b+c can be an integer of 5 to 22.

In another embodiment, a+b+c can be an integer of 6 to 22.

In another embodiment, a+b+c can be an integer of 7 to 22.

In another embodiment, a+b+c can be an integer of 8 to 22.

In one embodiment,
when a+b is 1, one of R₁ and R₂ is deuterium, and
when a+b is 2 or more, one of R₁ and R₂ is deuterium, and the rest are each independently deuterium or a C₆₋₂₀ aryl that is unsubstituted or substituted with at least one deuterium; or both R₁ and R₂ can be deuterium.

In another embodiment,
when a+b is 1, one of R₁ and R₂ is deuterium, and
when a+b is 2 or more, one of R₁ and R₂ is deuterium, and the rest are each independently deuterium or phenyl that is unsubstituted or substituted with 1 to 5 deuteriums; or both R₁ and R₂ are deuterium.

In addition, the substituent

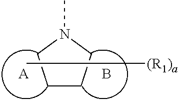

can be represented by any one of the following Chemical Formulas 2a to 2o:

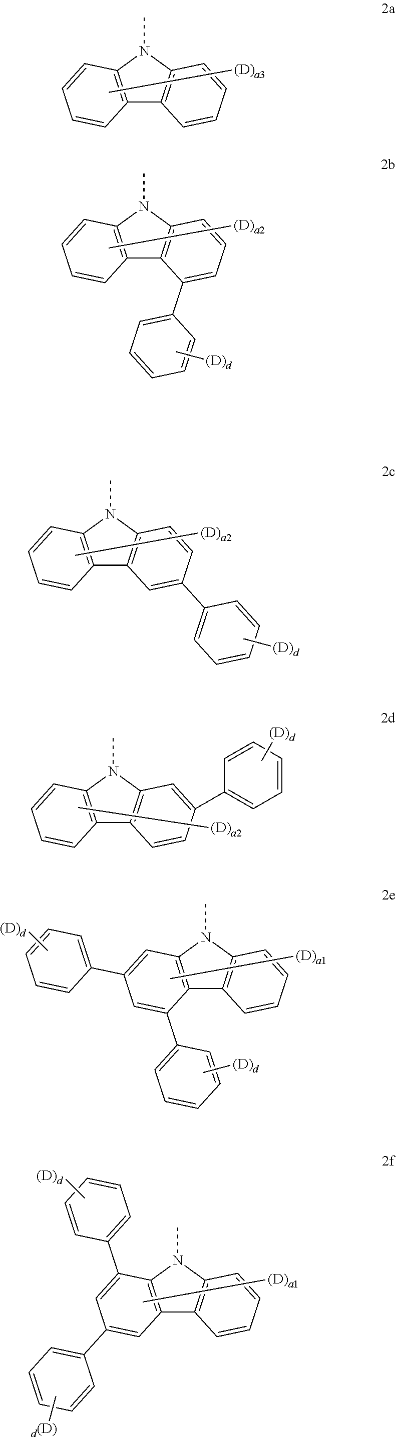

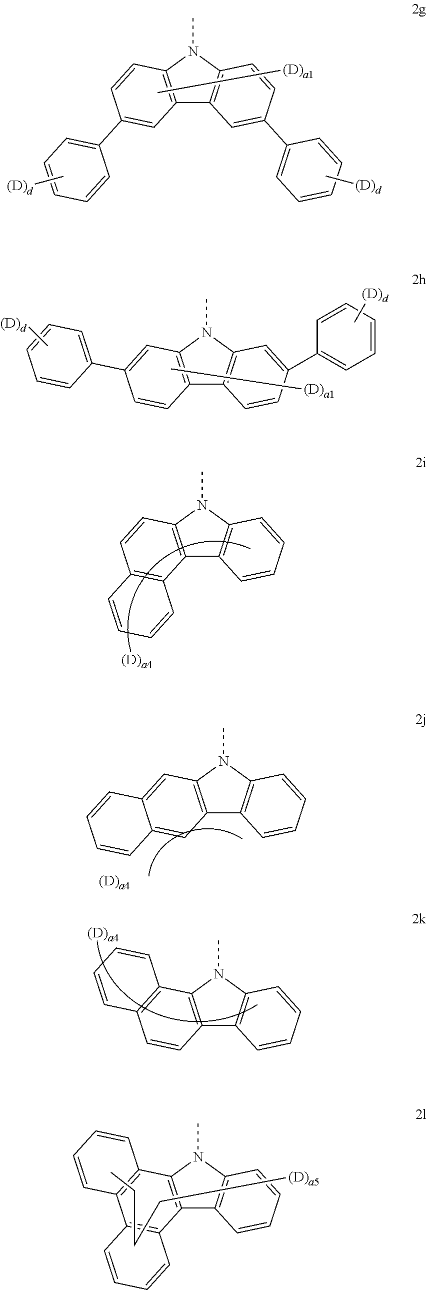

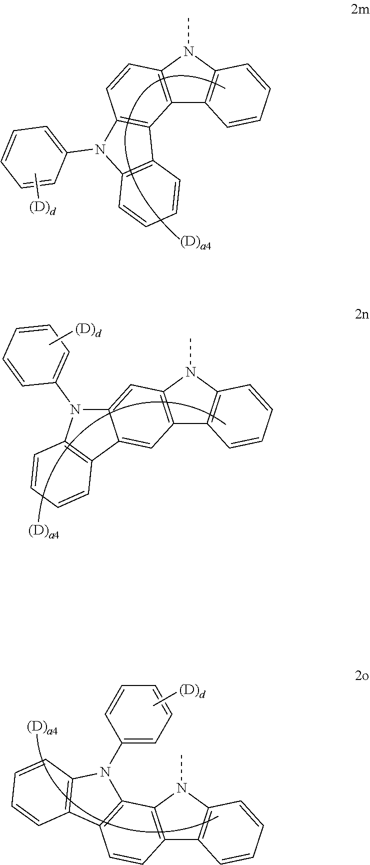

wherein, in Chemical Formulas 2a to 2o:
D is deuterium;
each d is independently an integer of 0 to 5;
a1 is an integer of 0 to 6;
a2 is an integer of 0 to 7;
a3 is an integer of 0 to 8;
a4 is an integer of 0 to 10; and 10
a5 is an integer of 0 to 12,
with the proviso that a1+b is 1 or more, or a2+b is 1 or more, or a3+b is 1 or more, a4+b is 1 or more, or a5+b is 1 or more.

At this time, a1 can be 0, 1, 2, 3, 4, 5, or 6,
a2 can be 0, 1, 2, 3, 4, 5, 6, or 7,
a3 can be 0, 1, 2, 3, 4, 5, 6, 7, or 8,
a4 can be 0, 1, 2, 3, 4, 5, 6, 7, 8, 9, or 10, and
a5 can be 0, 1, 2, 3, 4, 5, 6, 7, 8, 9, 10, 11, or 12.

More specifically, the substituent

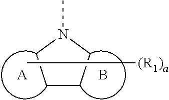

can be any one substituent selected from the group consisting of:

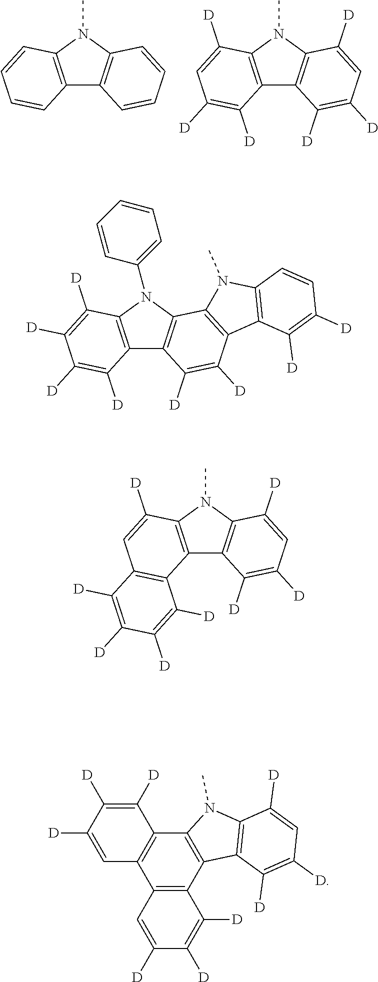

Further, the substituent

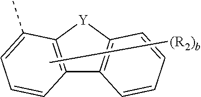

can be represented by any one of the following Chemical Formulas 3a to 3j:

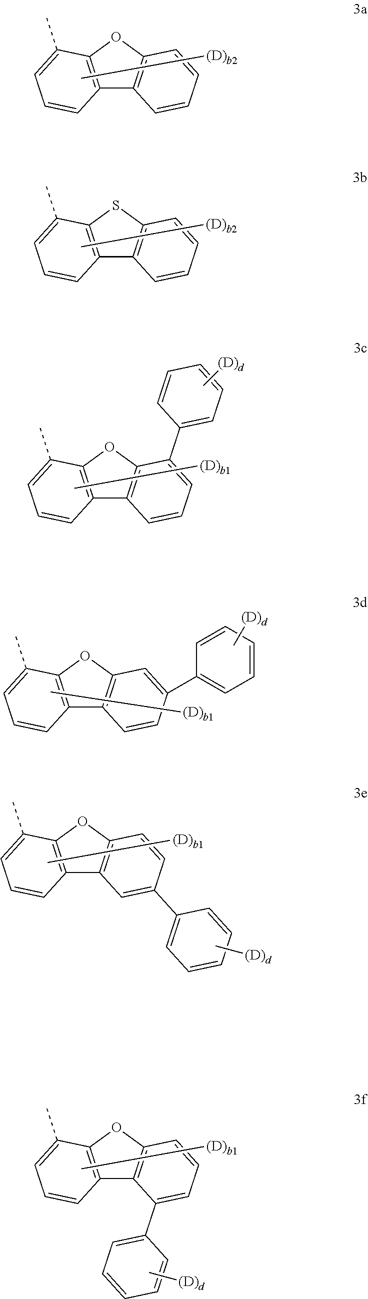

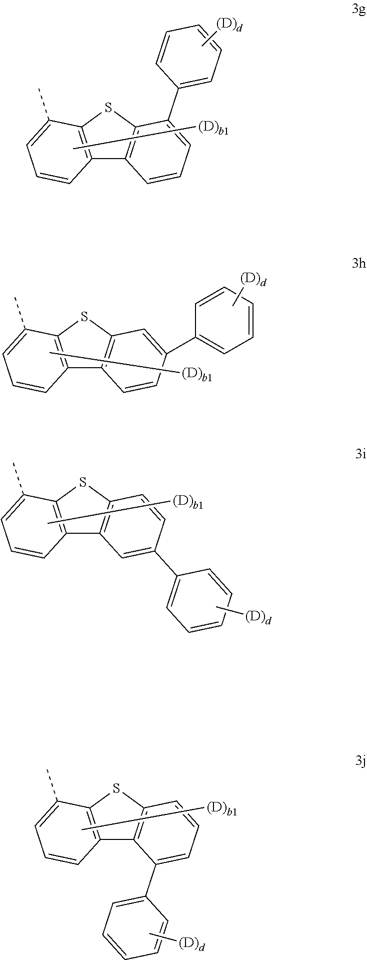

wherein, in Chemical Formulas 3a to 3j:
D is deuterium;
d is an integer of 0 to 5;
b1 is an integer of 0 to 6; and
b2 is an integer of 0 to 7,
with the proviso that a+b1 is 1 or more, or a+b2 is 1 or more.

However, when

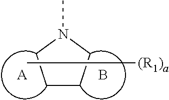

is represented by any one of the following Chemical Formulas 2a to 2o, and

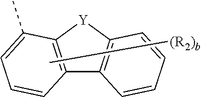

is represented by any one of the following Chemical Formulas 3a to 3j,
a1+b1 is 1 or more, or a2+b1 is 1 or more, or a3+b1 is 1 or more, or a4+b1 is 1 or more, or a5+b1 is 1 or more, or
a1+b2 is 1 or more, or a2+b2 is 1 or more, or a3+b2 is 1 or more, or a4+b2 is 1 or more, or a5+b2 is 1 or more.

At this time, b1 can be 0, 1, 2, 3, 4, 5, or 6, and
b2 can be 0, 1, 2, 3, 4, 5, 6, or 7.

More specifically, the substituent

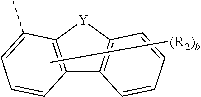

can be any one substituent selected from the group consisting of:

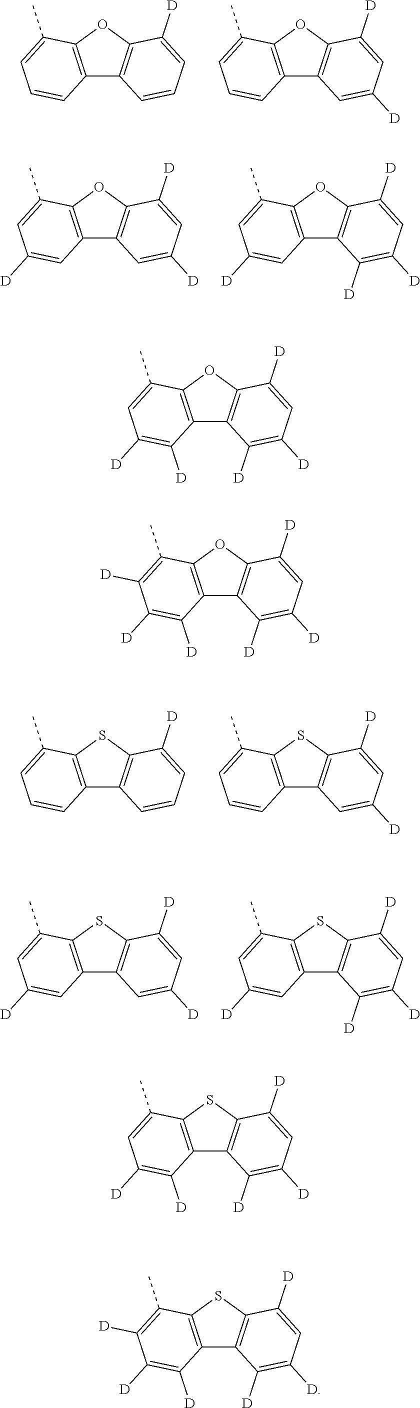

Further, the compound can be any one of the following Chemical Formulas 1-1 to 1-4:

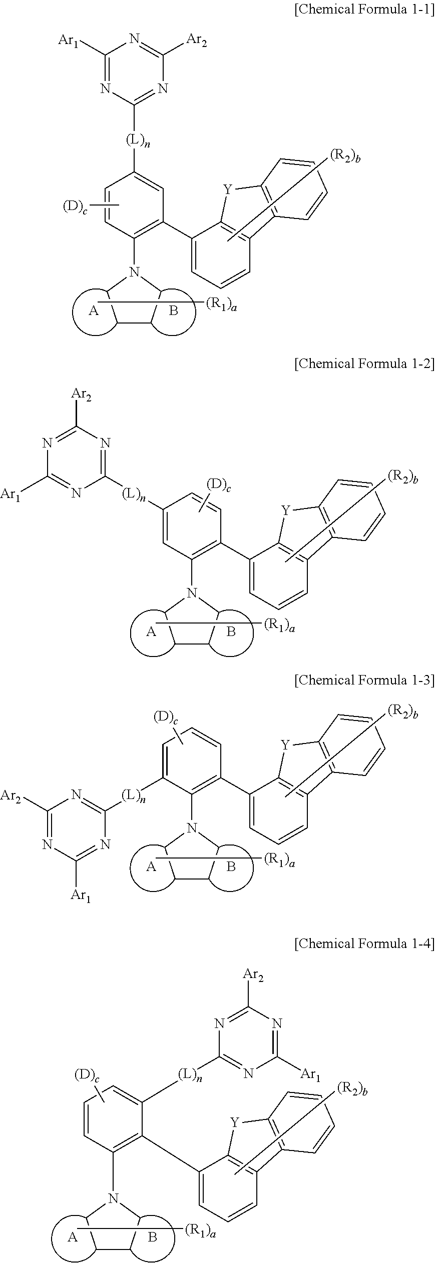

wherein, in Chemical Formulas 1-1 to 1-4:
A and B are each independently a benzene ring, naphthalene ring, phenanthrene ring, or carbazole ring which is fused with an adjacent pentagonal ring, and
Y, L, n, Ar₁, Ar₂, R₁, R₂, D, a, b and c are as defined in Chemical Formula 1.

Further, the compound can include 1 to 30 deuteriums. More specifically, the compound can include 1 or more, 2 or more, 3 or more, 4 or more, 5 or more, 6 or more, 7 or more, 8 or more, 9 or more, 10 or more, 11 or more, 12 or more, 13 or more, 14 or more, or 15 or more, and 30 or less, 29 or less, 28 or less, 27 or less, 26 or less, 25 or less, 24 or less, 23 or less, 22 or less, 21 or less, 20 or less, 19 or less, or 18 or less deuteriums.

Meanwhile, representative examples of the compound of Chemical Formula 1 are as follows:

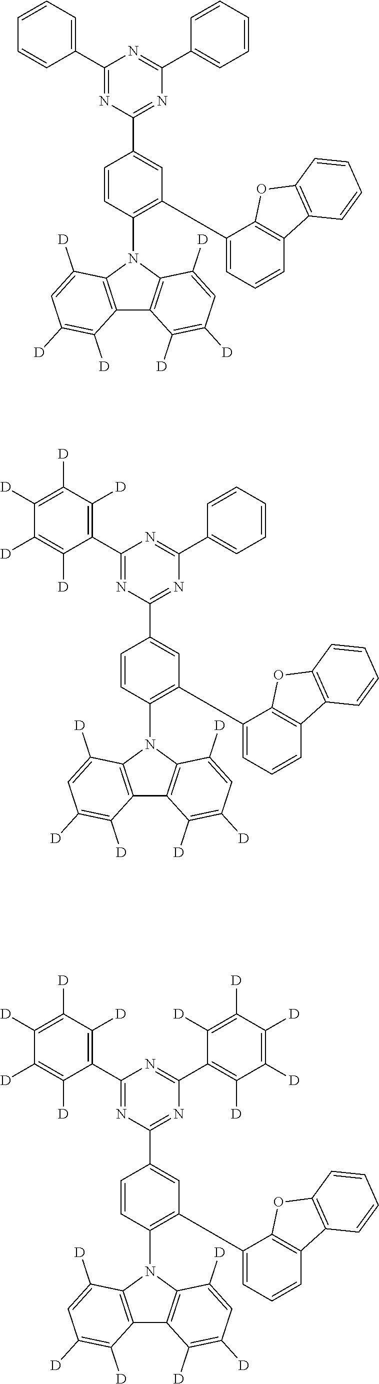 [SMILES: [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3c([2H])c([2H])c([2H])c([2H])c3[2H])n2)cc1-c1cccc2c1oc1ccccc12 ; [2H]c1cc([2H])c2c(c1[2H])c1c([2H])c([2H])cc([2H])c1n2-c1ccc(-c2nc(-c3c([2H])c([2H])c([2H])c([2H])c3[2H])nc(-c3c([2H])c([2H])c([2H])c([2H])c3[2H])n2)cc1-c1cccc2c1oc1ccccc12]

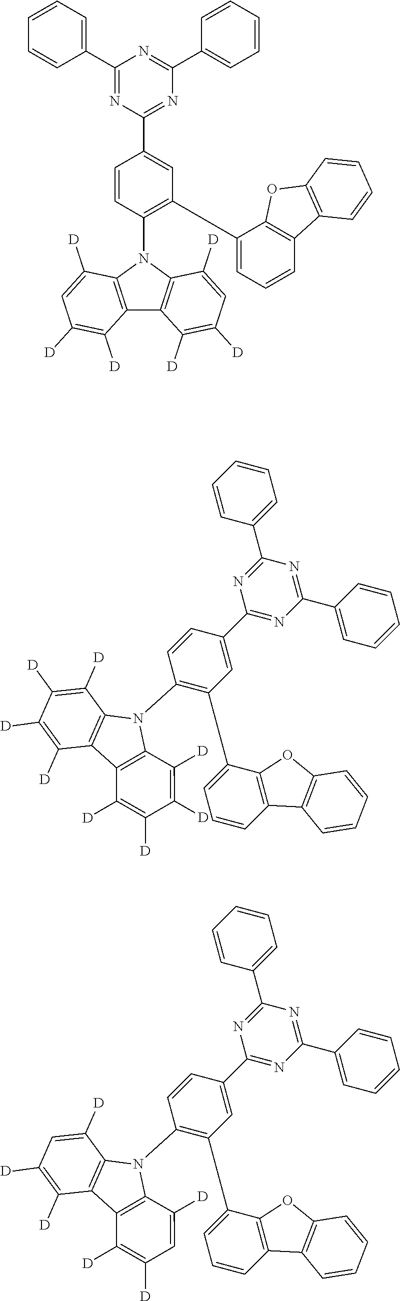 [SMILES: [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1oc1ccccc12 ; [2H]c1c([2H])c([2H])c2c(c1[2H])c1c([2H])c([2H])c([2H])c([2H])c1n2-c1ccc(-c2nc(-c3ccccc3)nc(-c3ccccc3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1oc1ccccc12]

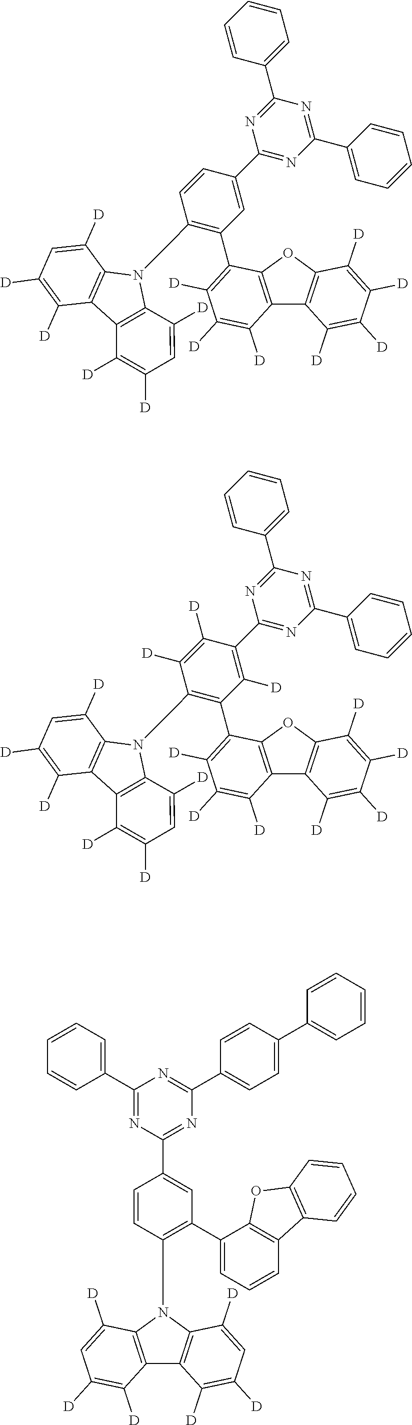 [SMILES: [2H]c1cc([2H])c2c(c1[2H])c1c([2H])c([2H])cc([2H])c1n2-c1ccc(-c2nc(-c3ccccc3)nc(-c3ccccc3)n2)cc1-c1c([2H])c([2H])c([2H])c2c1oc1c([2H])c([2H])c([2H])c([2H])c12 ; [2H]c1cc([2H])c2c(c1[2H])c1c([2H])c([2H])cc([2H])c1n2-c1c([2H])c([2H])c(-c2nc(-c3ccccc3)nc(-c3ccccc3)n2)c([2H])c1-c1c([2H])c([2H])c([2H])c2c1oc1c([2H])c([2H])c([2H])c([2H])c12 ; [2H]c1cc([2H])c2c(c1[2H])c1c([2H])c([2H])cc([2H])c1n2-c1ccc(-c2nc(-c3ccccc3)nc(-c3ccc(-c4ccccc4)cc3)n2)cc1-c1cccc2c1oc1ccccc12]

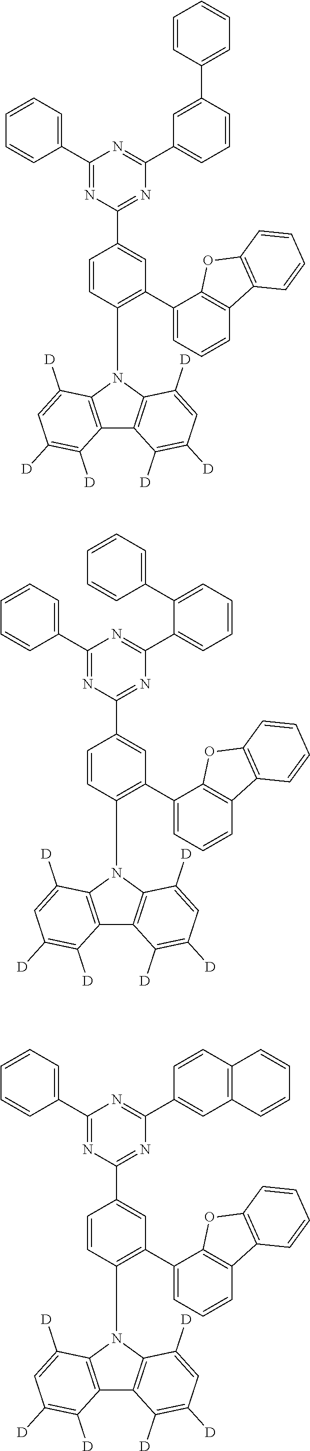 [SMILES: [2H]c1cc([2H])c2c(c1[2H])c1c([2H])c([2H])cc([2H])c1n2-c1ccc(-c2nc(-c3ccccc3)nc(-c3cccc(-c4ccccc4)c3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3ccccc3-c3ccccc3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3ccc4ccccc4c3)n2)cc1-c1cccc2c1oc1ccccc12]

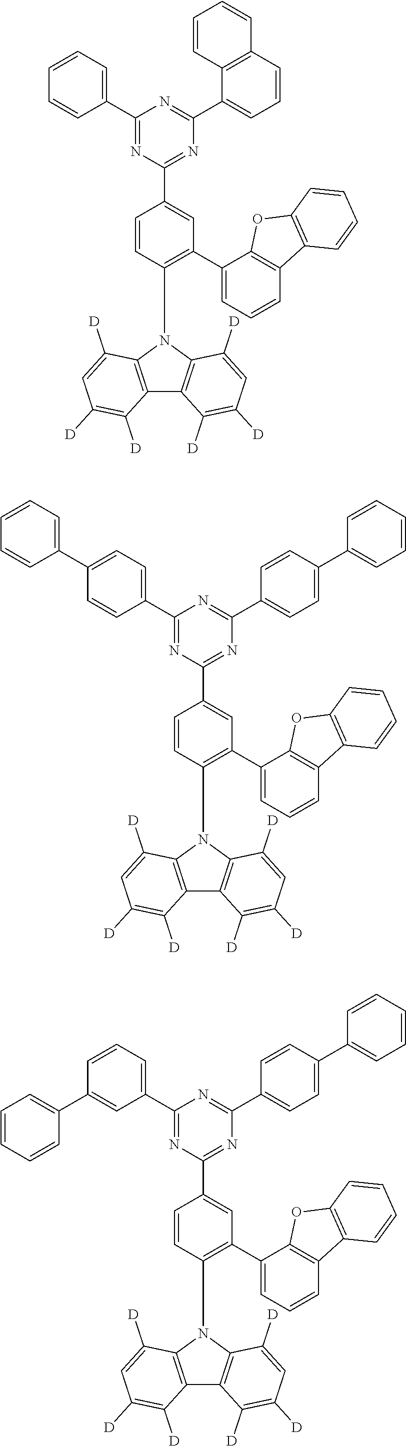 [SMILES: [2H]c1cc([2H])c2c(c1[2H])c1c([2H])c([2H])cc([2H])c1n2-c1ccc(-c2nc(-c3ccccc3)nc(-c3cccc4ccccc34)n2)cc1-c1cccc2c1oc1ccccc12 ; [2H]c1cc([2H])c2c(c1[2H])c1c([2H])c([2H])cc([2H])c1n2-c1ccc(-c2nc(-c3ccc(-c4ccccc4)cc3)nc(-c3ccc(-c4ccccc4)cc3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccc(-c4ccccc4)cc3)nc(-c3cccc(-c4ccccc4)c3)n2)cc1-c1cccc2c1oc1ccccc12]

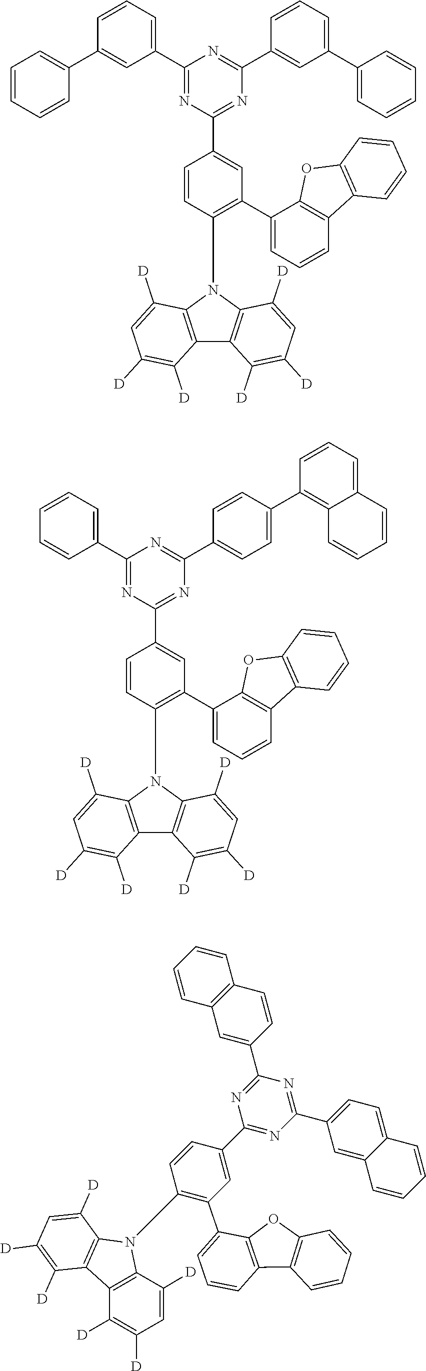 [SMILES: [2H]c1cc([2H])c2c(c1[2H])c1c([2H])c([2H])cc([2H])c1n2-c1ccc(-c2nc(-c3cccc(-c4ccccc4)c3)nc(-c3cccc(-c4ccccc4)c3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3ccc(-c4cccc5ccccc45)cc3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccc4ccccc4c3)nc(-c3ccc4ccccc4c3)n2)cc1-c1cccc2c1oc1ccccc12]

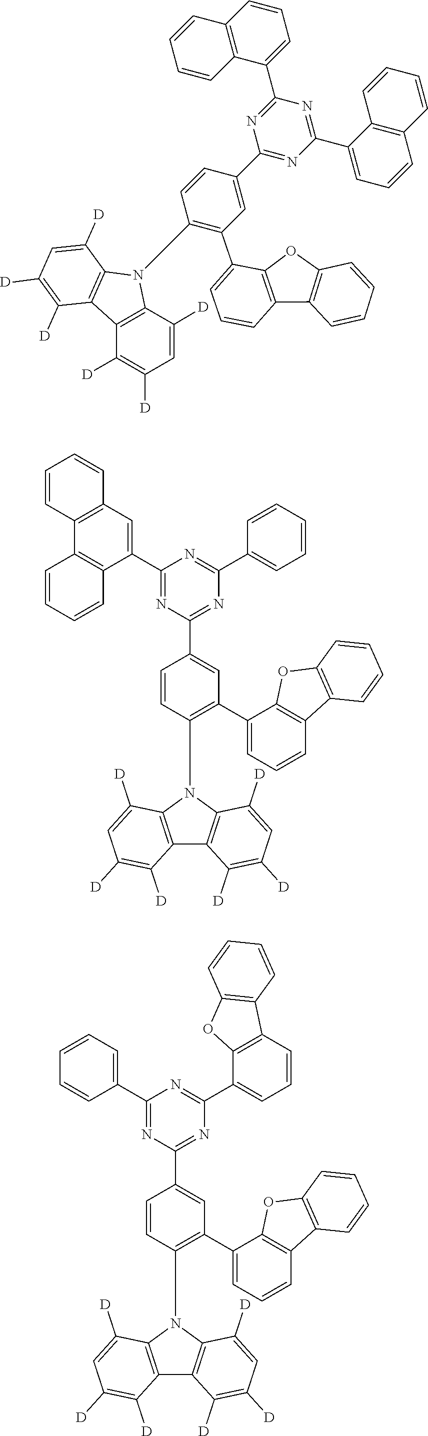

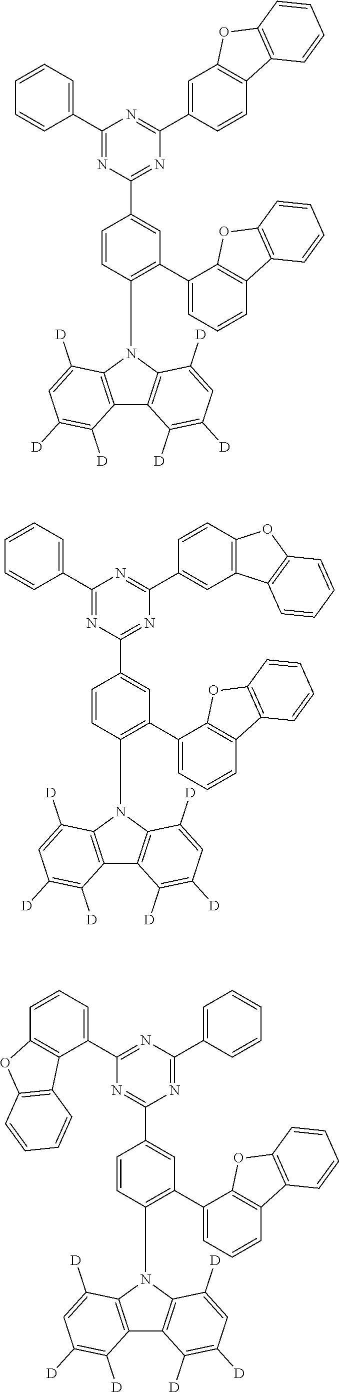 [SMILES: [2H]c1cc([2H])c2c(c1[2H])c1c([2H])c([2H])cc([2H])c1n2-c1ccc(-c2nc(-c3ccccc3)nc(-c3ccc4c(c3)oc3ccccc34)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3ccc4oc5ccccc5c4c3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3cccc4oc5ccccc5c34)n2)cc1-c1cccc2c1oc1ccccc12]

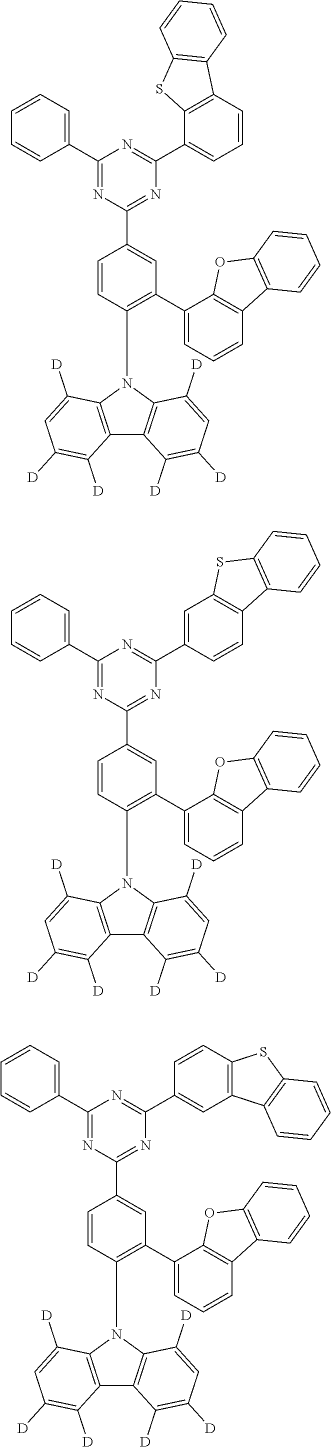 [SMILES: [2H]c1cc([2H])c2c(c1[2H])c1c([2H])c([2H])cc([2H])c1n2-c1ccc(-c2nc(-c3ccccc3)nc(-c3cccc4c3sc3ccccc34)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3ccc4c(c3)sc3ccccc34)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3ccc4sc5ccccc5c4c3)n2)cc1-c1cccc2c1oc1ccccc12]

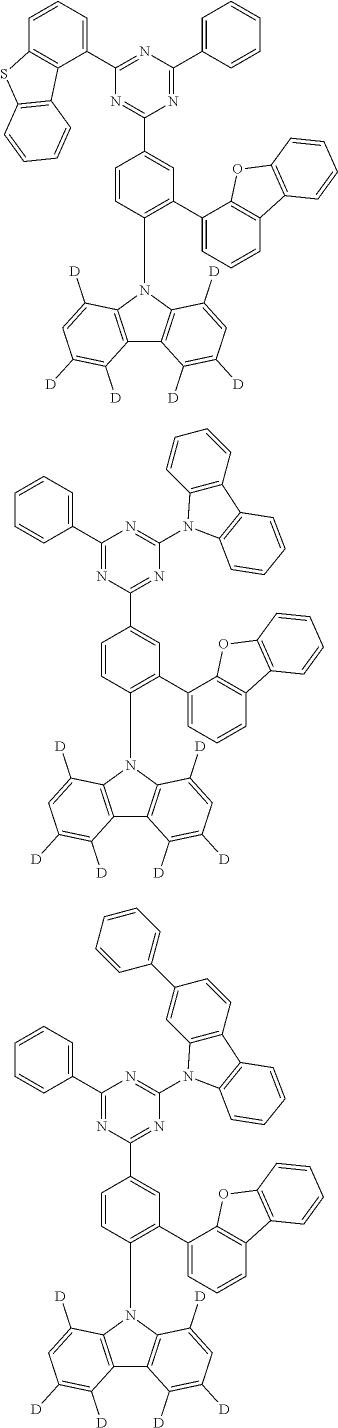

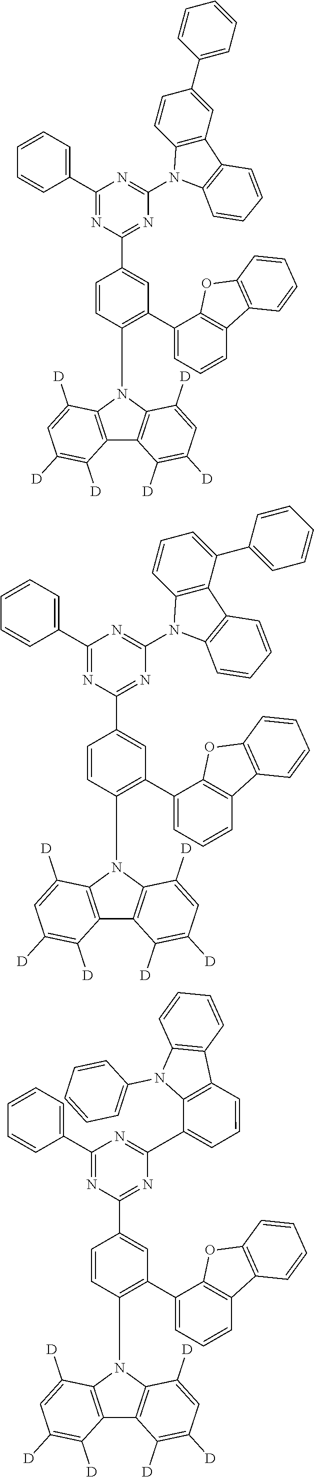

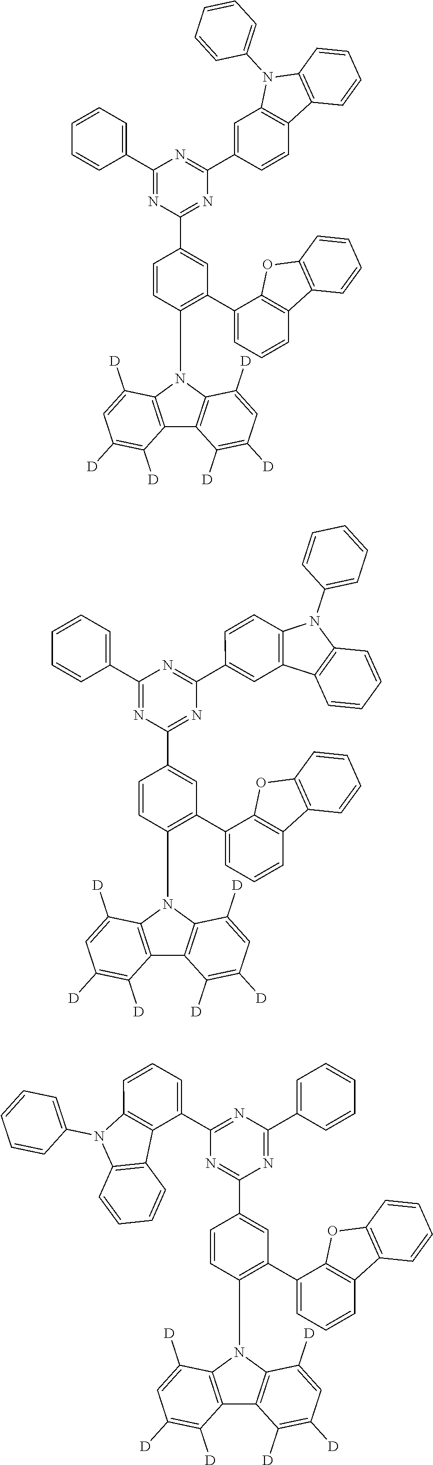 [SMILES: [2H]c1cc([2H])c2c(c1[2H])c1c([2H])c([2H])cc([2H])c1n2-c1ccc(-c2nc(-c3ccccc3)nc(-c3ccc4c5ccccc5n(-c5ccccc5)c4c3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3ccc4c(c3)c3ccccc3n4-c3ccccc3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3cccc4c3c3ccccc3n4-c3ccccc3)n2)cc1-c1cccc2c1oc1ccccc12]

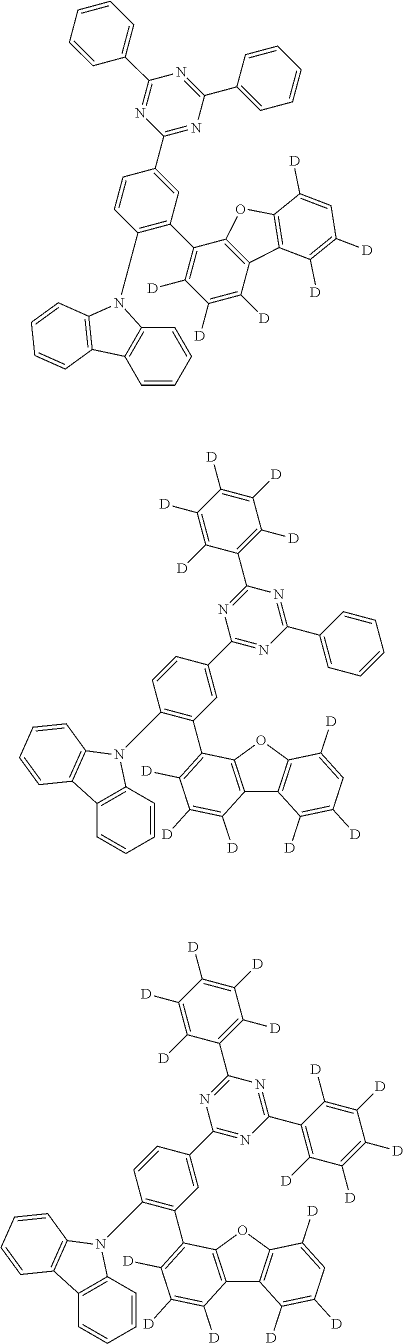 [SMILES: [2H]c1cc([2H])c2oc3c(-c4cc(-c5nc(-c6ccccc6)nc(-c6ccccc6)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6c([2H])c([2H])c([2H])c([2H])c6[2H])n5)ccc4-n4c5ccccc5c5ccccc54)c([2H])c([2H])c([2H])c3c2c1[2H] ; [2H]c1cc([2H])c2oc3c(-c4cc(-c5nc(-c6c([2H])c([2H])c([2H])c([2H])c6[2H])nc(-c6c([2H])c([2H])c([2H])c([2H])c6[2H])n5)ccc4-n4c5ccccc5c5ccccc54)c([2H])c([2H])c([2H])c3c2c1[2H]]

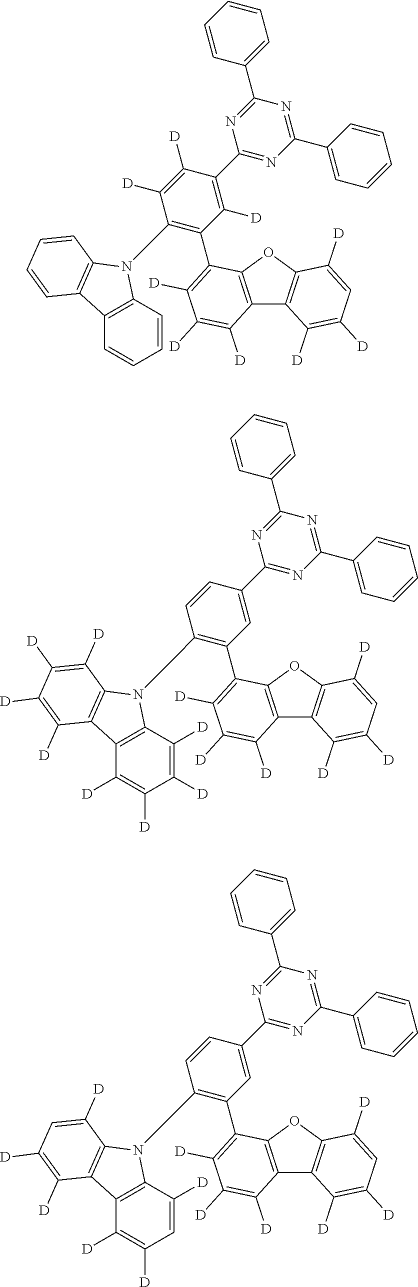 [SMILES: [2H]c1cc([2H])c2oc3c(-c4c([2H])c(-c5nc(-c6ccccc6)nc(-c6ccccc6)n5)c([2H])c([2H])c4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6ccccc6)n5)ccc4-n4c5c([2H])c([2H])c([2H])c([2H])c5c5c([2H])c([2H])c([2H])c([2H])c54)c([2H])c([2H])c([2H])c3c2c1[2H] ; [2H]c1cc([2H])c2oc3c(-c4cc(-c5nc(-c6ccccc6)nc(-c6ccccc6)n5)ccc4-n4c5c([2H])cc([2H])c([2H])c5c5c([2H])c([2H])cc([2H])c54)c([2H])c([2H])c([2H])c3c2c1[2H]]

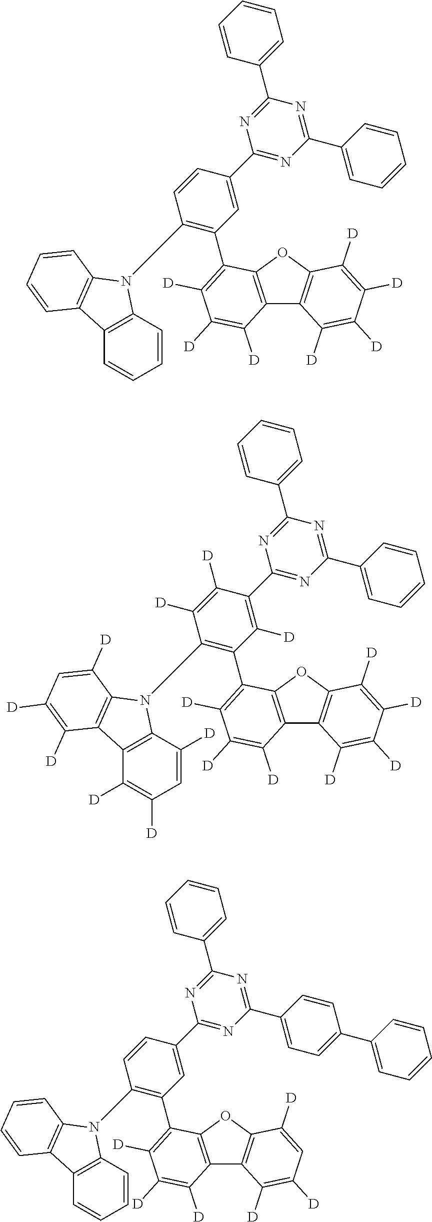 [SMILES: [2H]c1c([2H])c([2H])c2c(oc3c(-c4cc(-c5nc(-c6ccccc6)nc(-c6ccccc6)n5)ccc4-n4c5ccccc5c5ccccc54)c([2H])c([2H])c([2H])c32)c1[2H] ; [2H]c1cc([2H])c2c(c1[2H])c1c([2H])c([2H])cc([2H])c1n2-c1c([2H])c([2H])c(-c2nc(-c3ccccc3)nc(-c3ccccc3)n2)c([2H])c1-c1c([2H])c([2H])c([2H])c2c1oc1c([2H])c([2H])c([2H])c([2H])c12 ; [2H]c1cc([2H])c2oc3c(-c4cc(-c5nc(-c6ccccc6)nc(-c6ccc(-c7ccccc7)cc6)n5)ccc4-n4c5ccccc5c5ccccc54)c([2H])c([2H])c([2H])c3c2c1[2H]]

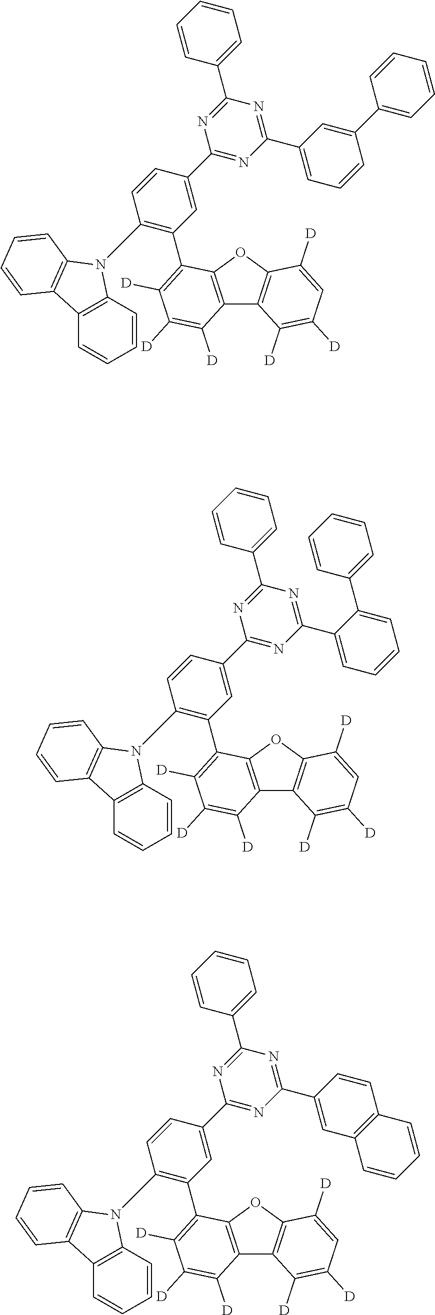 [SMILES: [2H]c1cc([2H])c2oc3c(-c4cc(-c5nc(-c6ccccc6)nc(-c6cccc(-c7ccccc7)c6)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6ccccc6-c6ccccc6)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6ccc7ccccc7c6)n5)ccc4-n4c5ccccc5c5ccccc54)c([2H])c([2H])c([2H])c3c2c1[2H]]

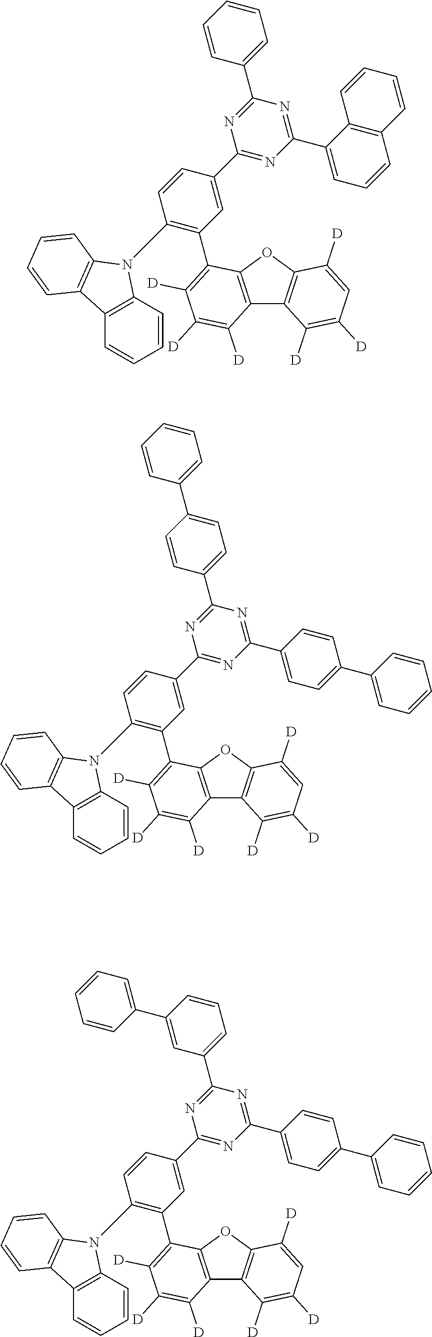 [SMILES: [2H]c1cc([2H])c2oc3c(-c4cc(-c5nc(-c6ccccc6)nc(-c6cccc7ccccc67)n5)ccc4-n4c5ccccc5c5ccccc54)c([2H])c([2H])c([2H])c3c2c1[2H] ; [2H]c1cc([2H])c2oc3c(-c4cc(-c5nc(-c6ccc(-c7ccccc7)cc6)nc(-c6ccc(-c7ccccc7)cc6)n5)ccc4-n4c5ccccc5c5ccccc54)c([2H])c([2H])c([2H])c3c2c1[2H] ; [2H]c1cc([2H])c2oc3c(-c4cc(-c5nc(-c6ccc(-c7ccccc7)cc6)nc(-c6cccc(-c7ccccc7)c6)n5)ccc4-n4c5ccccc5c5ccccc54)c([2H])c([2H])c([2H])c3c2c1[2H]]

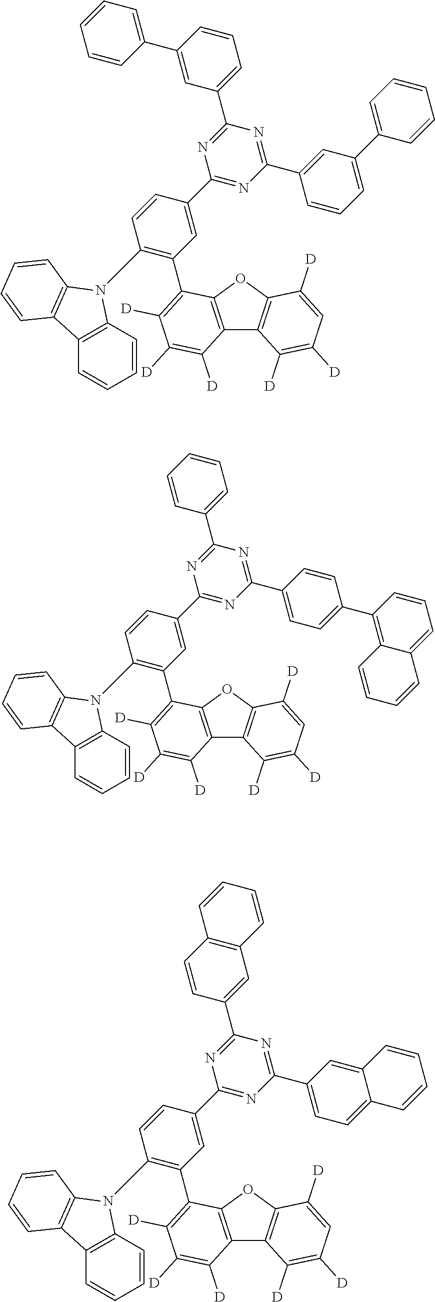 [SMILES: [2H]c1cc([2H])c2oc3c(-c4cc(-c5nc(-c6cccc(-c7ccccc7)c6)nc(-c6cccc(-c7ccccc7)c6)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6ccc(-c7cccc8ccccc78)cc6)n5)ccc4-n4c5ccccc5c5ccccc54)c([2H])c([2H])c([2H])c3c2c1[2H] ; [2H]c1cc([2H])c2oc3c(-c4cc(-c5nc(-c6ccc7ccccc7c6)nc(-c6ccc7ccccc7c6)n5)ccc4-n4c5ccccc5c5ccccc54)c([2H])c([2H])c([2H])c3c2c1[2H]]

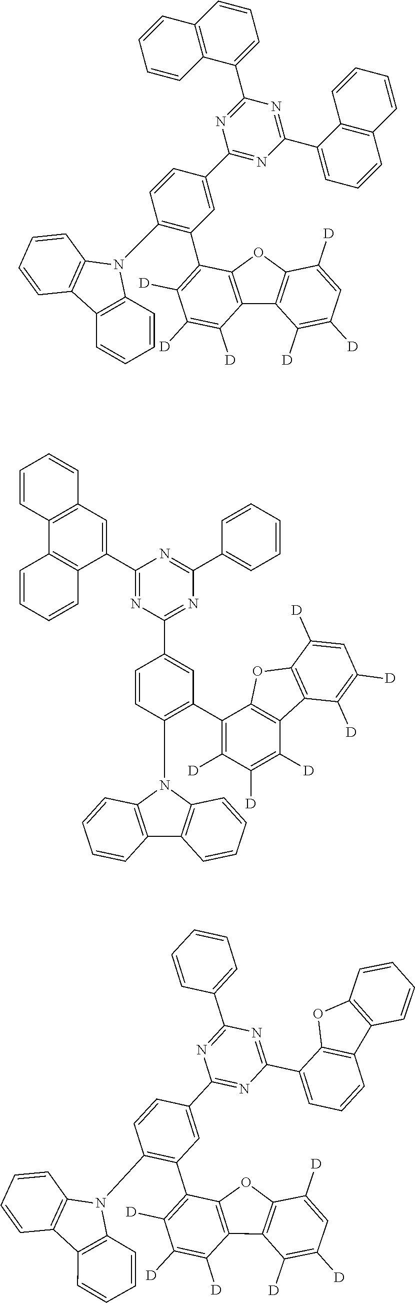 [SMILES: [2H]c1cc([2H])c2oc3c(-c4cc(-c5nc(-c6cccc7ccccc67)nc(-c6cccc7ccccc67)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6cc7ccccc7c7ccccc67)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6cccc7c6oc6ccccc67)n5)ccc4-n4c5ccccc5c5ccccc54)c([2H])c([2H])c([2H])c3c2c1[2H]]

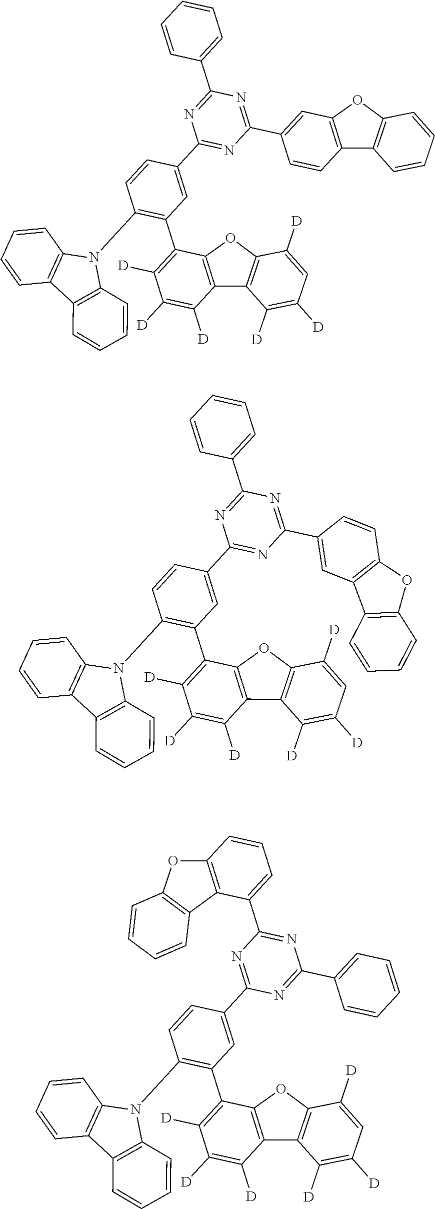 [SMILES: [2H]c1cc([2H])c2oc3c(-c4cc(-c5nc(-c6ccccc6)nc(-c6ccc7c(c6)oc6ccccc67)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6ccc7oc8ccccc8c7c6)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6cccc7oc8ccccc8c67)n5)ccc4-n4c5ccccc5c5ccccc54)c([2H])c([2H])c([2H])c3c2c1[2H]]

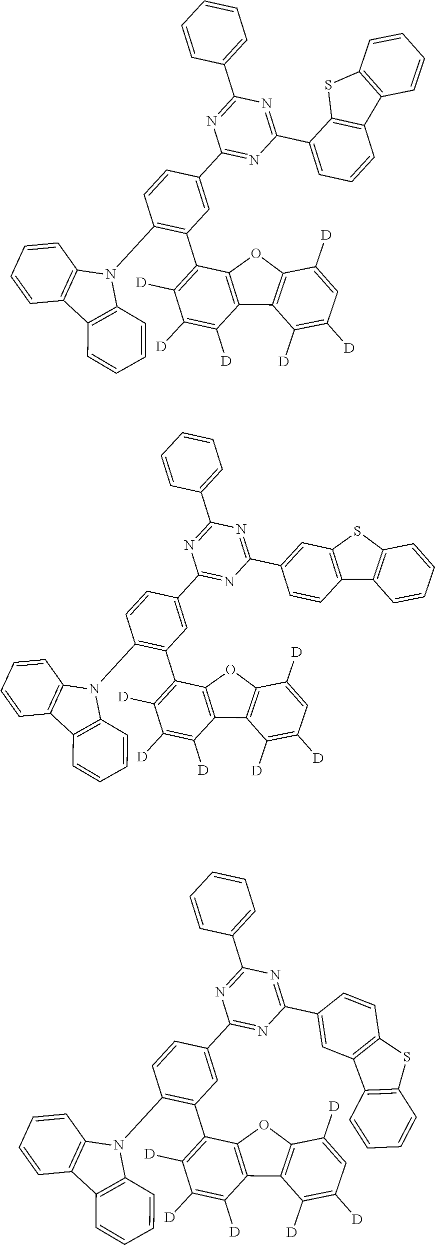 [SMILES: [2H]c1cc([2H])c2oc3c(-c4cc(-c5nc(-c6ccccc6)nc(-c6cccc7c6sc6ccccc67)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6ccc7c(c6)sc6ccccc67)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6ccc7sc8ccccc8c7c6)n5)ccc4-n4c5ccccc5c5ccccc54)c([2H])c([2H])c([2H])c3c2c1[2H]]

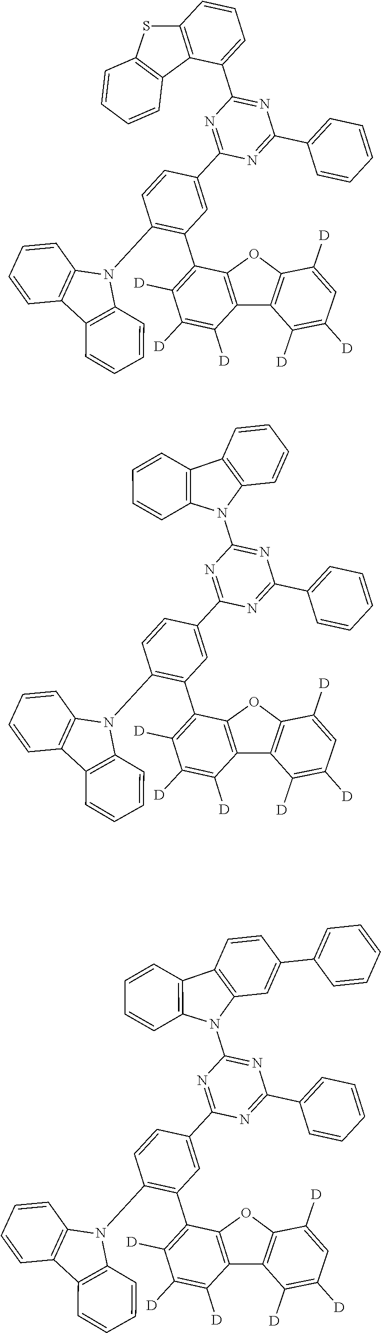 [SMILES: [2H]c1cc([2H])c2oc3c(-c4cc(-c5nc(-c6ccccc6)nc(-c6cccc7sc8ccccc8c67)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-n6c7ccccc7c7ccccc76)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-n6c7ccccc7c7ccc(-c8ccccc8)cc76)n5)ccc4-n4c5ccccc5c5ccccc54)c([2H])c([2H])c([2H])c3c2c1[2H]]

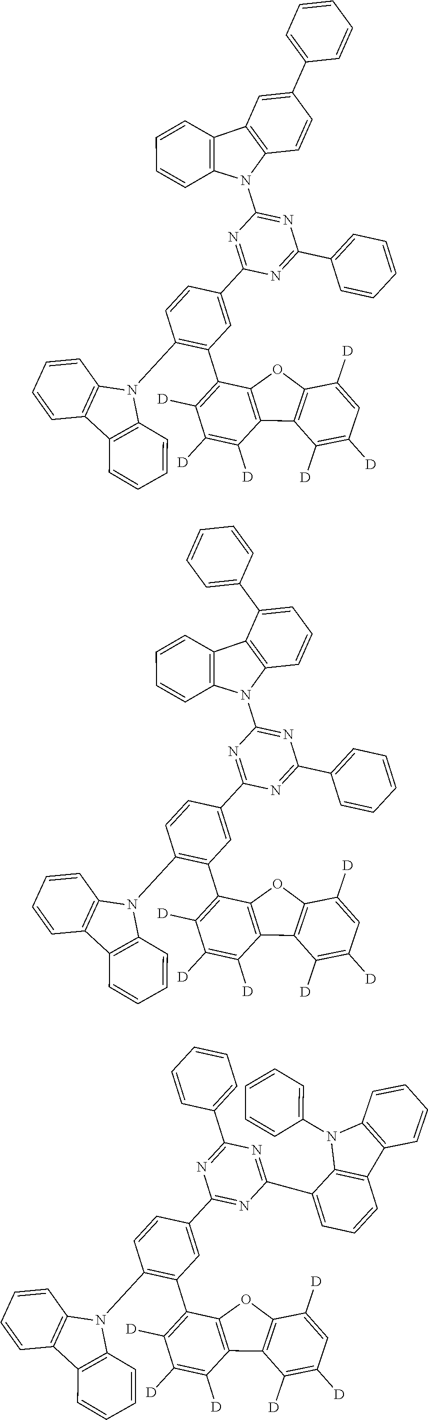 [SMILES: [2H]c1cc([2H])c2oc3c(-c4cc(-c5nc(-c6ccccc6)nc(-n6c7ccccc7c7cc(-c8ccccc8)ccc76)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-n6c7ccccc7c7c(-c8ccccc8)cccc76)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6cccc7c8ccccc8n(-c8ccccc8)c67)n5)ccc4-n4c5ccccc5c5ccccc54)c([2H])c([2H])c([2H])c3c2c1[2H]]

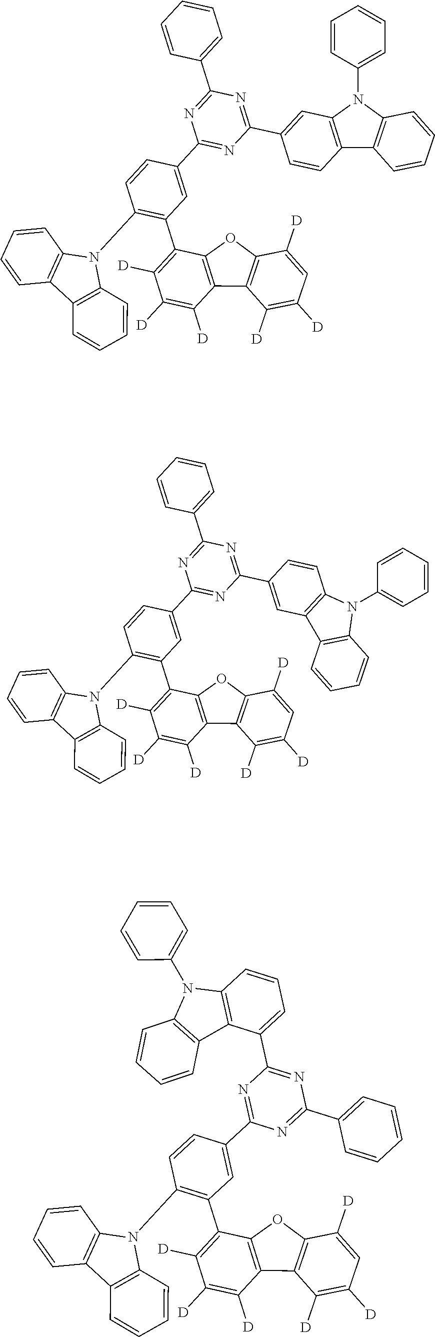 [SMILES: [2H]c1cc([2H])c2oc3c(-c4cc(-c5nc(-c6ccccc6)nc(-c6ccc7c8ccccc8n(-c8ccccc8)c7c6)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6ccc7c(c6)c6ccccc6n7-c6ccccc6)n5)ccc4-n4c5ccccc5c5ccccc54)c([2H])c([2H])c([2H])c3c2c1[2H] ; [2H]c1cc([2H])c2oc3c(-c4cc(-c5nc(-c6ccccc6)nc(-c6cccc7c6c6ccccc6n7-c6ccccc6)n5)ccc4-n4c5ccccc5c5ccccc54)c([2H])c([2H])c([2H])c3c2c1[2H]]

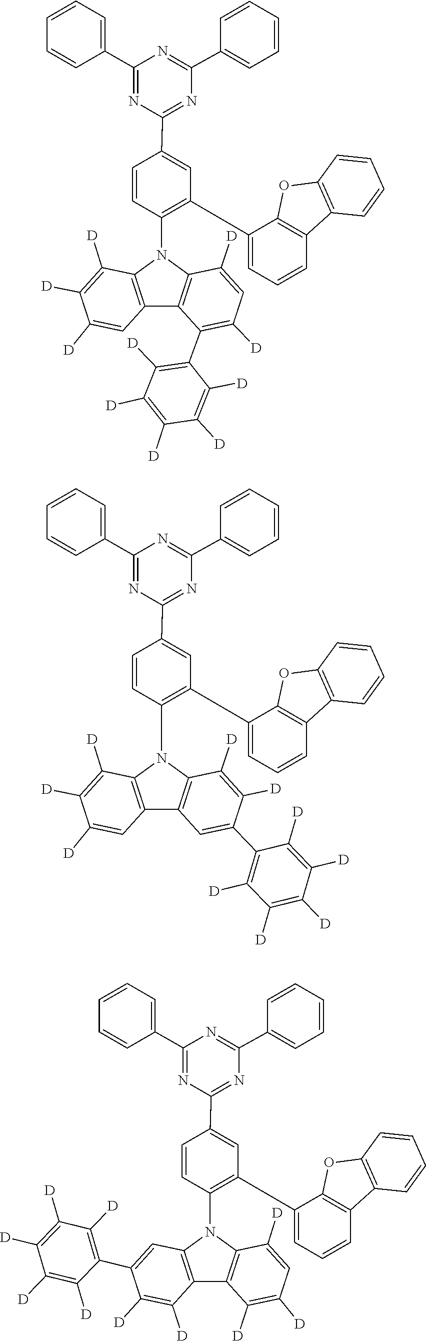 [SMILES: [2H]c1cc2c3c(-c4c([2H])c([2H])c([2H])c([2H])c4[2H])c([2H])cc([2H])c3n(-c3ccc(-c4nc(-c5ccccc5)nc(-c5ccccc5)n4)cc3-c3cccc4c3oc3ccccc34)c2c([2H])c1[2H] ; [2H]c1cc2c3cc(-c4c([2H])c([2H])c([2H])c([2H])c4[2H])c([2H])c([2H])c3n(-c3ccc(-c4nc(-c5ccccc5)nc(-c5ccccc5)n4)cc3-c3cccc4c3oc3ccccc34)c2c([2H])c1[2H] ; [2H]c1cc([2H])c2c(c1[2H])c1c([2H])c([2H])c(-c3c([2H])c([2H])c([2H])c([2H])c3[2H])cc1n2-c1ccc(-c2nc(-c3ccccc3)nc(-c3ccccc3)n2)cc1-c1cccc2c1oc1ccccc12]

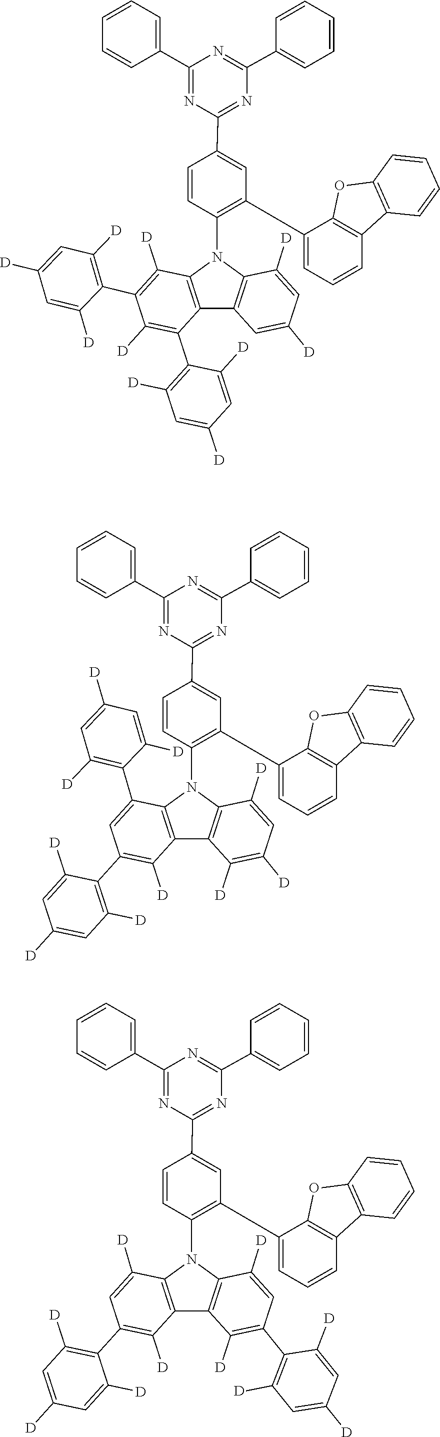 [SMILES: [2H]c1cc([2H])c(-c2c([2H])c(-c3c([2H])cc([2H])cc3[2H])c3c4cc([2H])cc([2H])c4n(-c4ccc(-c5nc(-c6ccccc6)nc(-c6ccccc6)n5)cc4-c4cccc5c4oc4ccccc45)c3c2[2H])c([2H])c1 ; [2H]c1cc([2H])c(-c2cc(-c3c([2H])cc([2H])cc3[2H])c3c(c2[2H])c2c([2H])c([2H])cc([2H])c2n3-c2ccc(-c3nc(-c4ccccc4)nc(-c4ccccc4)n3)cc2-c2cccc3c2oc2ccccc23)c([2H])c1 ; [2H]c1cc([2H])c(-c2cc([2H])c3c(c2[2H])c2c([2H])c(-c4c([2H])cc([2H])cc4[2H])cc([2H])c2n3-c2ccc(-c3nc(-c4ccccc4)nc(-c4ccccc4)n3)cc2-c2cccc3c2oc2ccccc23)c([2H])c1]

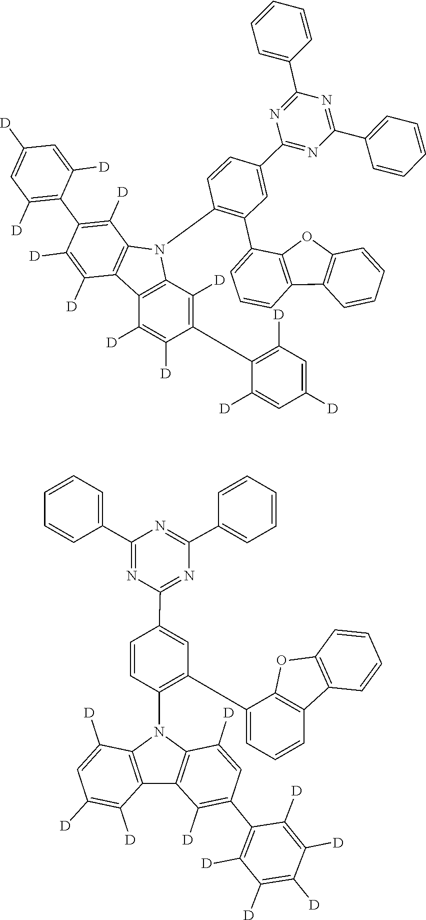 [SMILES: [2H]c1cc([2H])c(-c2c([2H])c([2H])c3c4c([2H])c([2H])c(-c5c([2H])cc([2H])cc5[2H])c([2H])c4n(-c4ccc(-c5nc(-c6ccccc6)nc(-c6ccccc6)n5)cc4-c4cccc5c4oc4ccccc45)c3c2[2H])c([2H])c1 ; [2H]c1cc([2H])c2c(c1[2H])c1c([2H])c(-c3c([2H])c([2H])c([2H])c([2H])c3[2H])cc([2H])c1n2-c1ccc(-c2nc(-c3ccccc3)nc(-c3ccccc3)n2)cc1-c1cccc2c1oc1ccccc12]

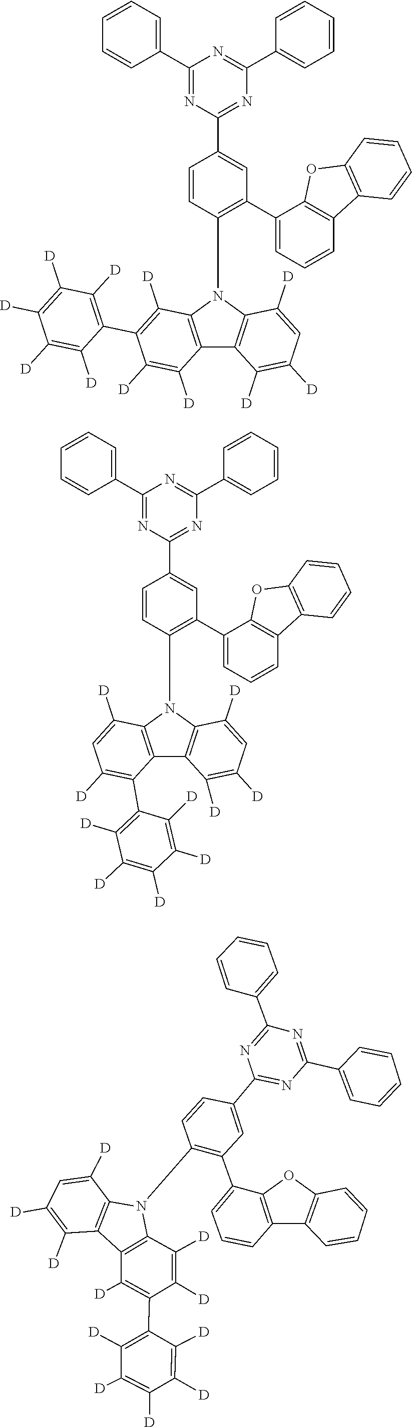

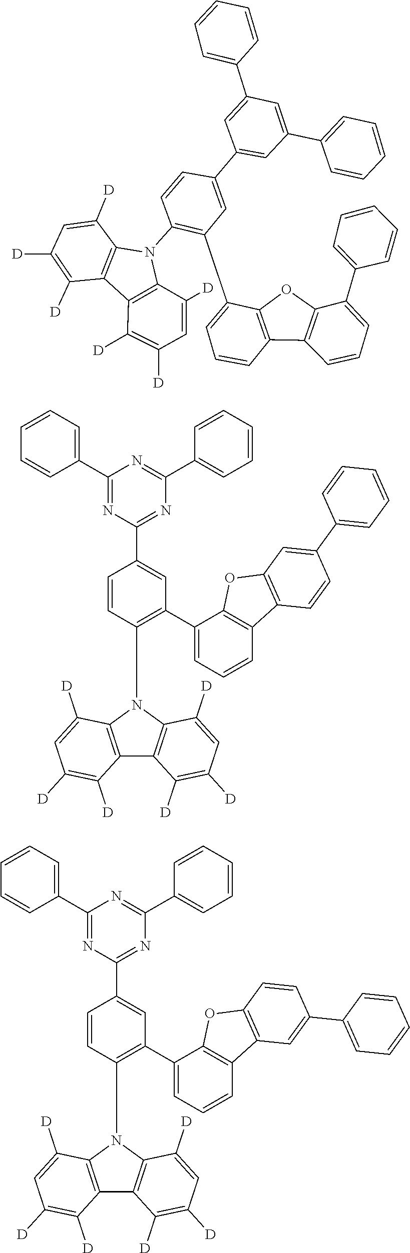 [SMILES: [2H]c1cc([2H])c2c(c1[2H])c1c([2H])c([2H])cc([2H])c1n2-c1ccc(-c2cc(-c3ccccc3)cc(-c3ccccc3)c2)cc1-c1cccc2c1oc1c(-c3ccccc3)cccc12 ; [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1oc1cc(-c3ccccc3)ccc12 ; [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1oc1ccc(-c3ccccc3)cc12]

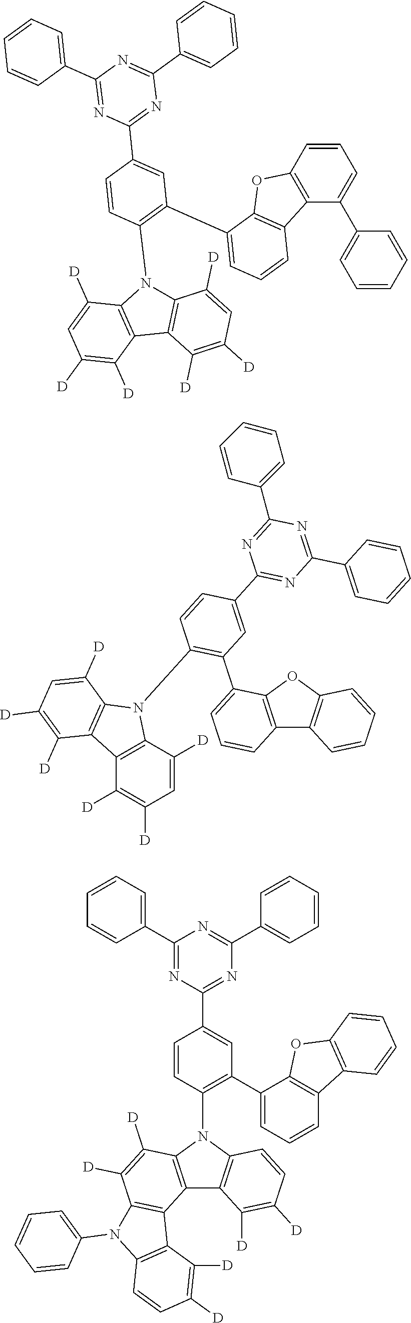

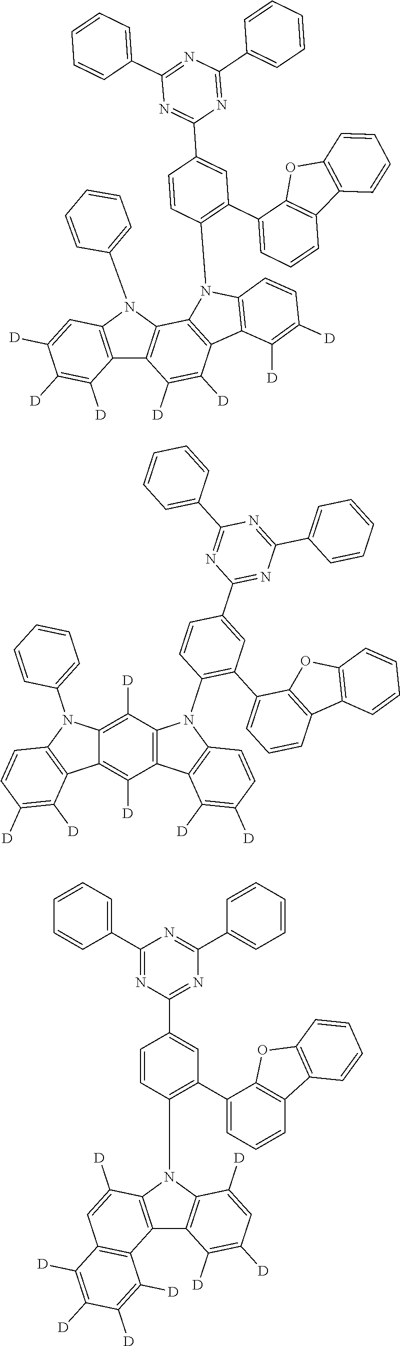

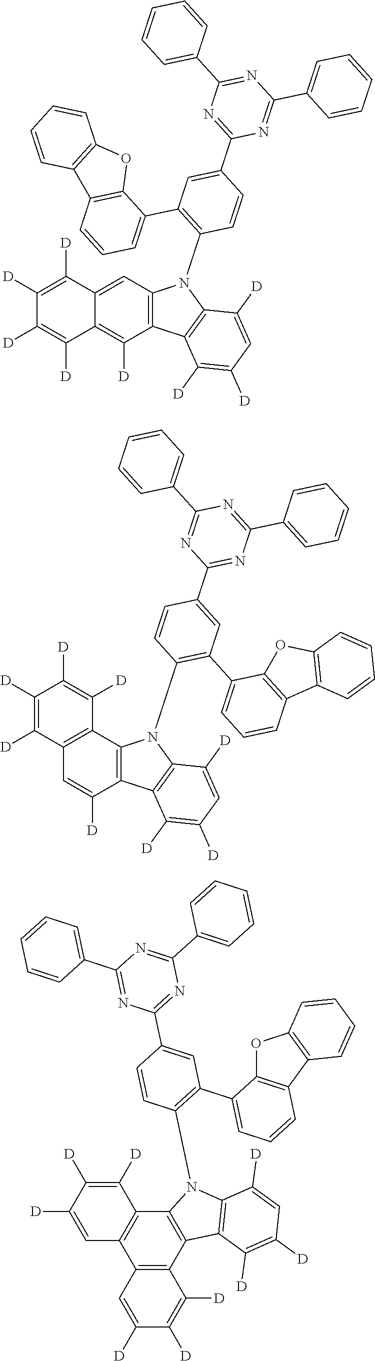

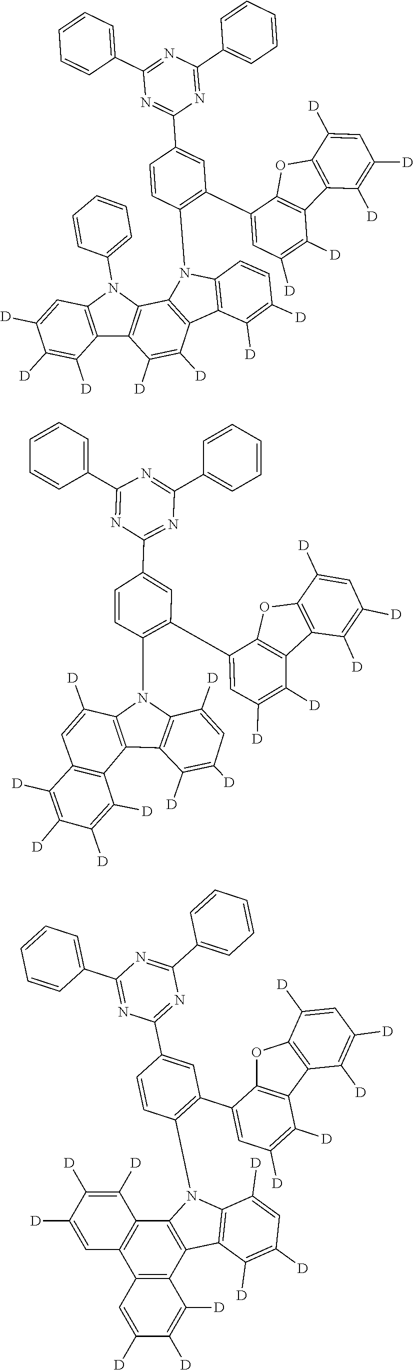

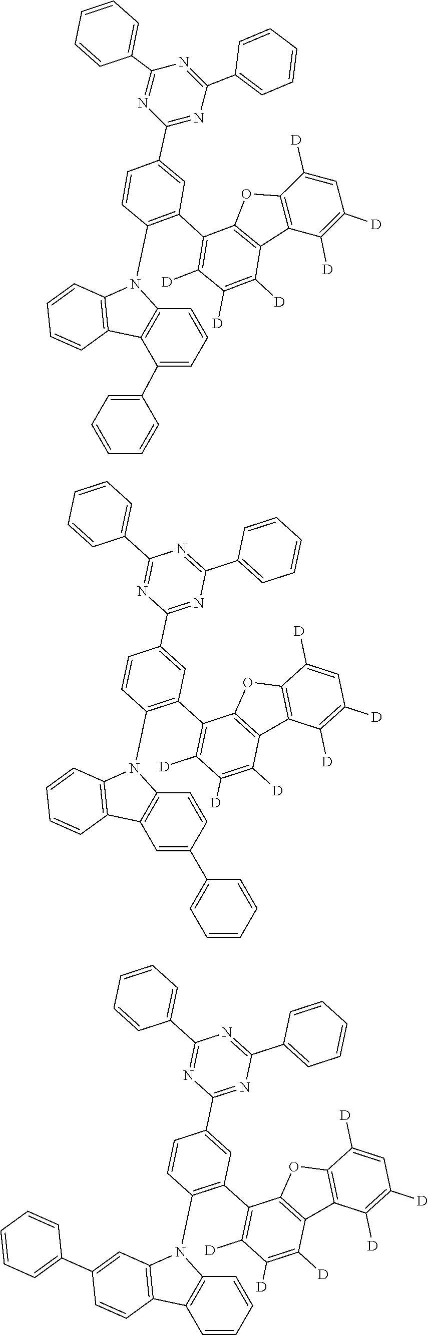

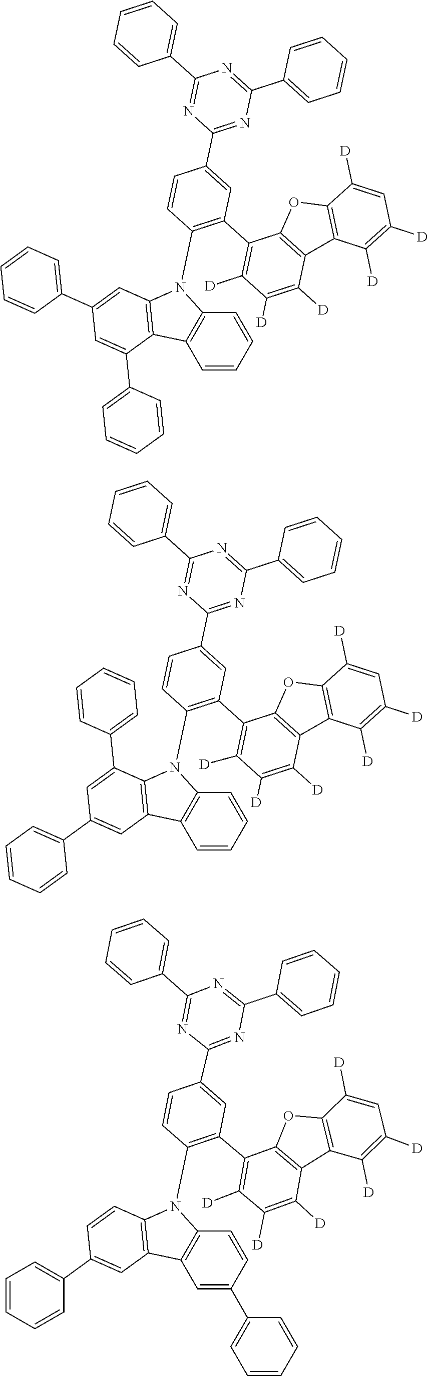

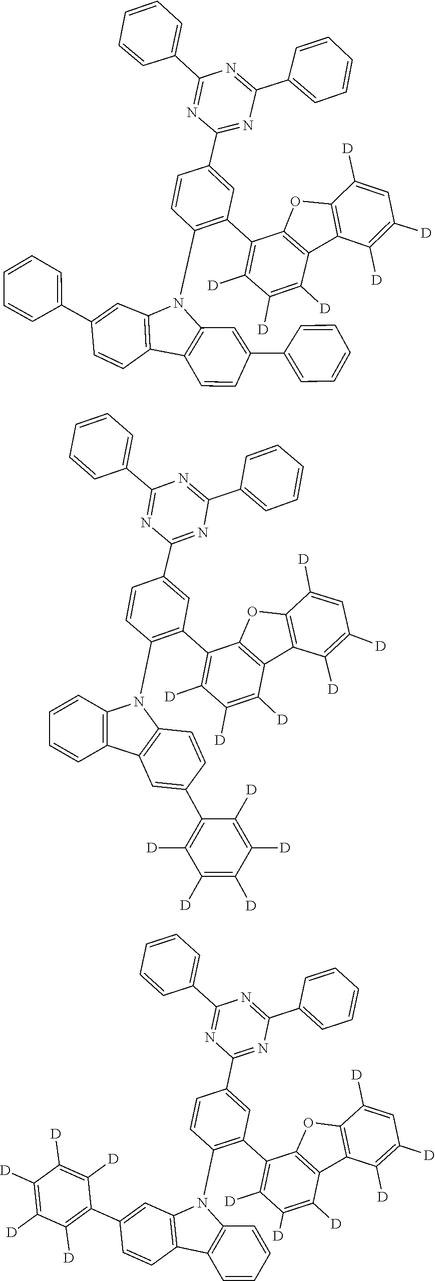

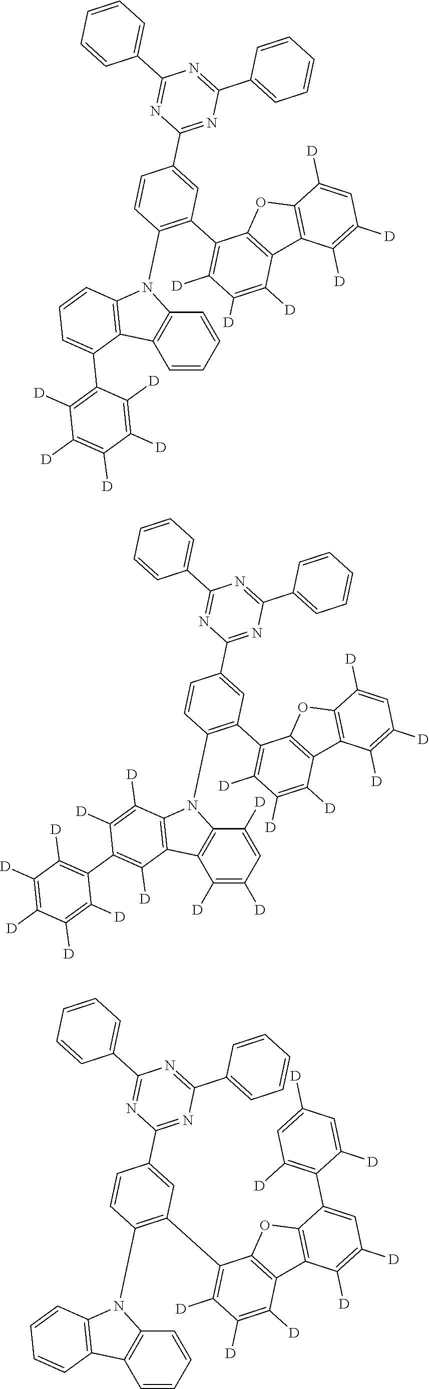 [SMILES: [2H]c1cc([2H])c2oc3c(-c4cc(-c5nc(-c6ccccc6)nc(-c6ccccc6)n5)ccc4-n4c5ccccc5c5c(-c6c([2H])c([2H])c([2H])c([2H])c6[2H])cccc54)c([2H])c([2H])c([2H])c3c2c1[2H] ; [2H]c1cc([2H])c2oc3c(-c4cc(-c5nc(-c6ccccc6)nc(-c6ccccc6)n5)ccc4-n4c5c([2H])cc([2H])c([2H])c5c5c([2H])c(-c6c([2H])c([2H])c([2H])c([2H])c6[2H])c([2H])c([2H])c54)c([2H])c([2H])c([2H])c3c2c1[2H] ; [2H]c1cc([2H])c(-c2cc([2H])c([2H])c3c2oc2c(-c4cc(-c5nc(-c6ccccc6)nc(-c6ccccc6)n5)ccc4-n4c5ccccc5c5ccccc54)c([2H])c([2H])c([2H])c23)c([2H])c1]

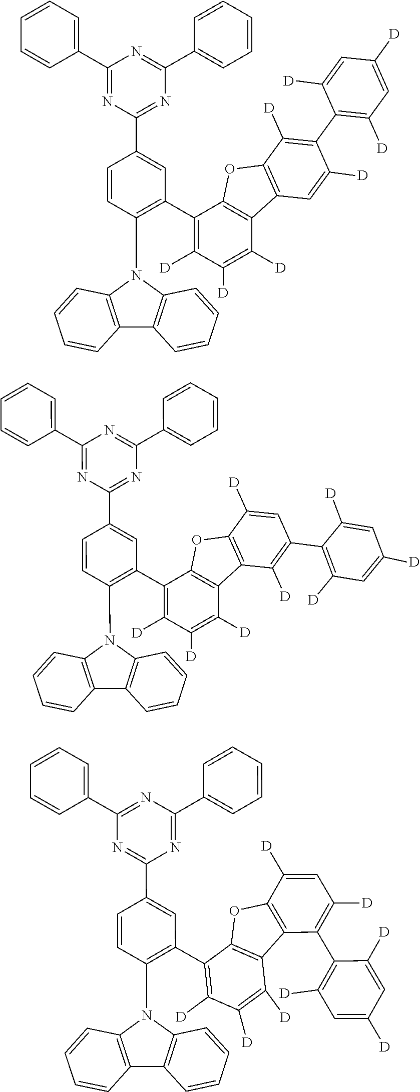

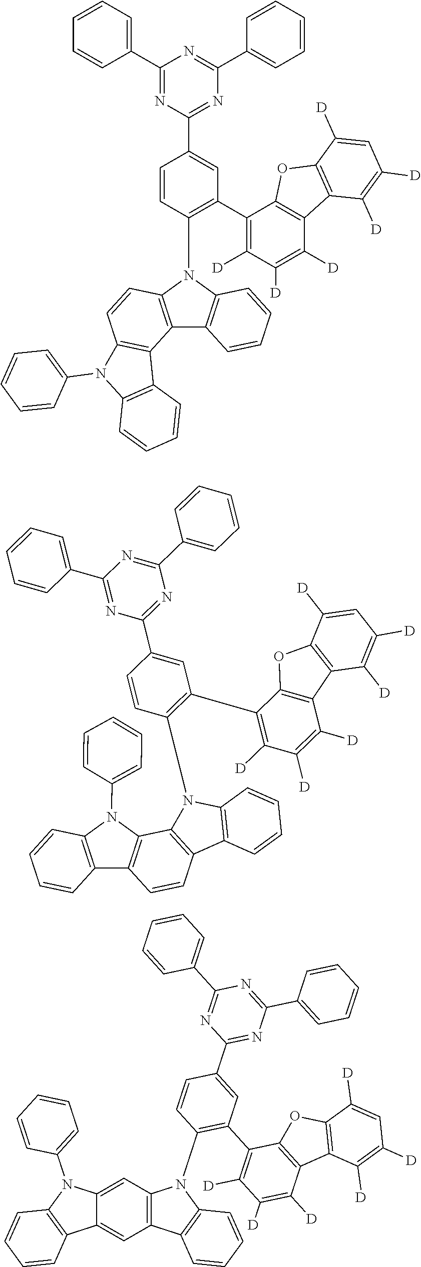

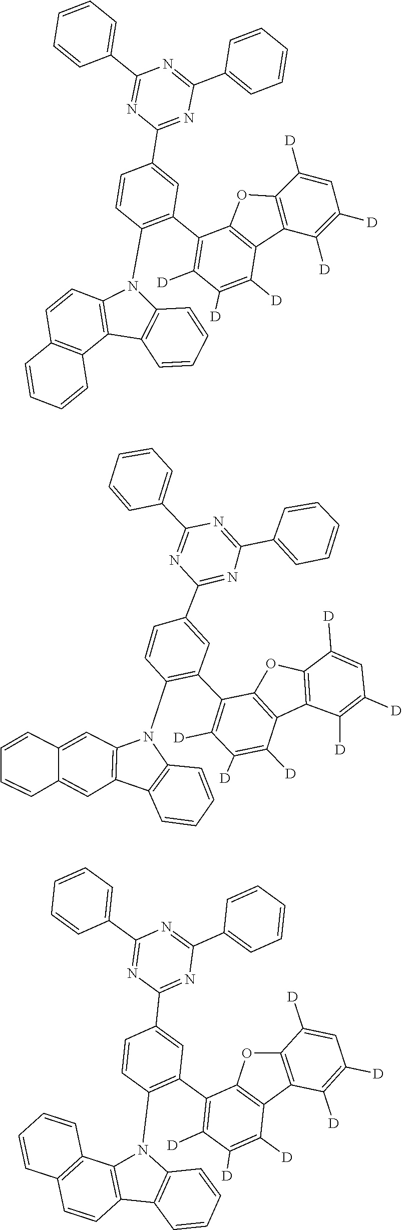 [SMILES: [2H]c1cc([2H])c2oc3c(-c4cc(-c5nc(-c6ccccc6)nc(-c6ccccc6)n5)ccc4-n4c5ccccc5c5c6ccccc6ccc54)c([2H])c([2H])c([2H])c3c2c1[2H] ; [2H]Cc1c([2H])c(-c2cc(-c3nc(-c4ccccc4)nc(-c4ccccc4)n3)ccc2-n2c3ccccc3c3cc4ccccc4cc32)c2oc3c([2H])cc([2H])c([2H])c3c2c1[2H] ; [2H]c1cc([2H])c2oc3c(-c4cc(-c5nc(-c6ccccc6)nc(-c6ccccc6)n5)ccc4-n4c5ccccc5c5ccc6ccccc6c54)c([2H])c([2H])c([2H])c3c2c1[2H]]

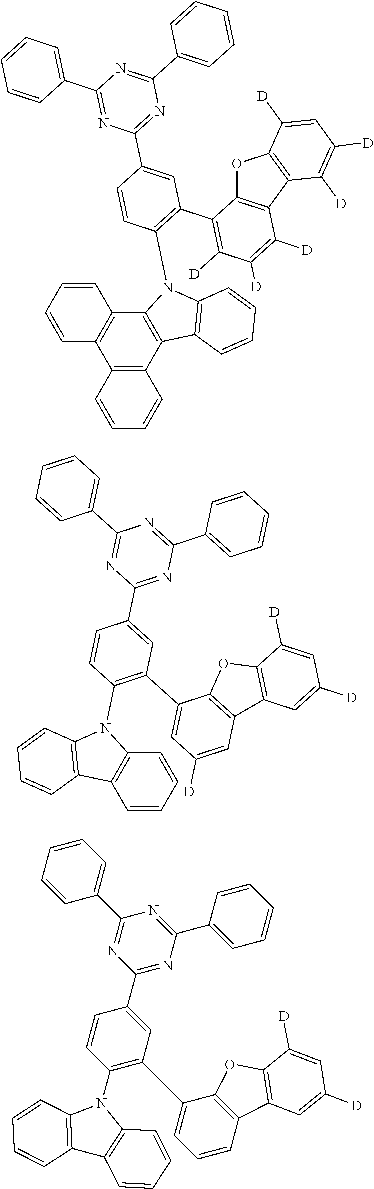 [SMILES: [2H]c1cc([2H])c2oc3c(-c4cc(-c5nc(-c6ccccc6)nc(-c6ccccc6)n5)ccc4-n4c5ccccc5c5c6ccccc6c6ccccc6c54)c([2H])c([2H])c([2H])c3c2c1[2H] ; [2H]c1cc([2H])c2oc3c(-c4cc(-c5nc(-c6ccccc6)nc(-c6ccccc6)n5)ccc4-n4c5ccccc5c5ccccc54)cc([2H])cc3c2c1 ; [2H]c1cc([2H])c2oc3c(-c4cc(-c5nc(-c6ccccc6)nc(-c6ccccc6)n5)ccc4-n4c5ccccc5c5ccccc54)cccc3c2c1]

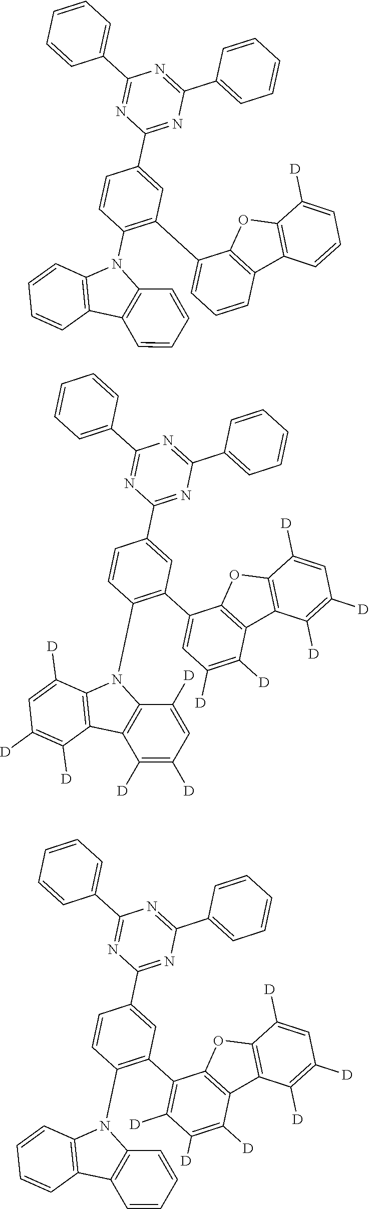 [SMILES: [2H]c1cccc2c1oc1c(-c3cc(-c4nc(-c5ccccc5)nc(-c5ccccc5)n4)ccc3-n3c4ccccc4c4ccccc43)cccc12 ; [2H]c1cc([2H])c2oc3c(-c4cc(-c5nc(-c6ccccc6)nc(-c6ccccc6)n5)ccc4-n4c5c([2H])cc([2H])c([2H])c5c5c([2H])c([2H])cc([2H])c54)cc([2H])c([2H])c3c2c1[2H] ; [2H]c1cc([2H])c2oc3c(-c4cc(-c5nc(-c6ccccc6)nc(-c6ccccc6)n5)ccc4-n4c5ccccc5c5ccccc54)c([2H])c([2H])c([2H])c3c2c1[2H]]

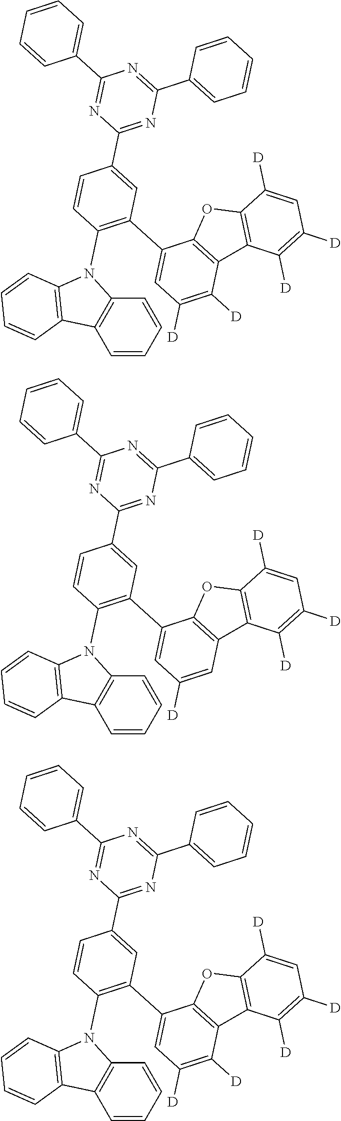

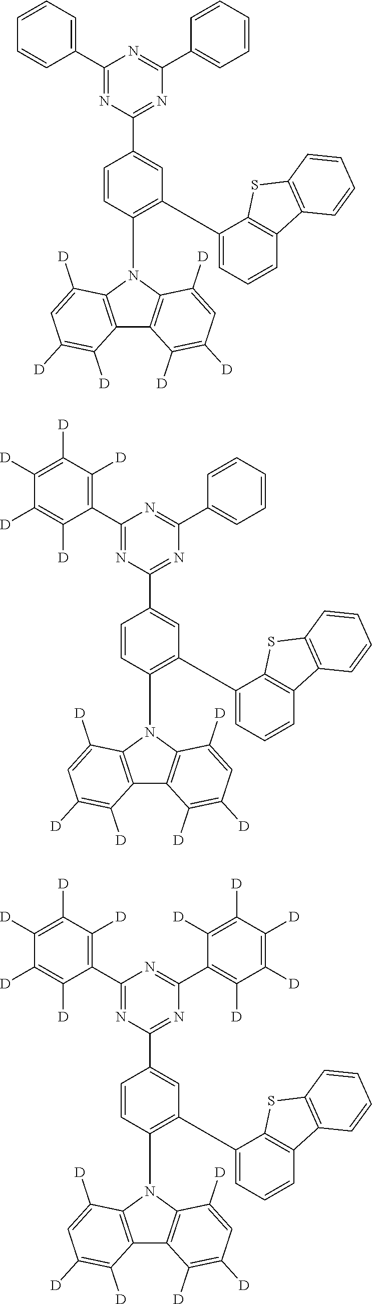 [SMILES: [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1sc1ccccc12 ; [2H]c1cc([2H])c2c(c1[2H])c1c([2H])c([2H])cc([2H])c1n2-c1ccc(-c2nc(-c3ccccc3)nc(-c3c([2H])c([2H])c([2H])c([2H])c3[2H])n2)cc1-c1cccc2c1sc1ccccc12 ; [2H]c1cc([2H])c2c(c1[2H])c1c([2H])c([2H])cc([2H])c1n2-c1ccc(-c2nc(-c3c([2H])c([2H])c([2H])c([2H])c3[2H])nc(-c3c([2H])c([2H])c([2H])c([2H])c3[2H])n2)cc1-c1cccc2c1sc1ccccc12]

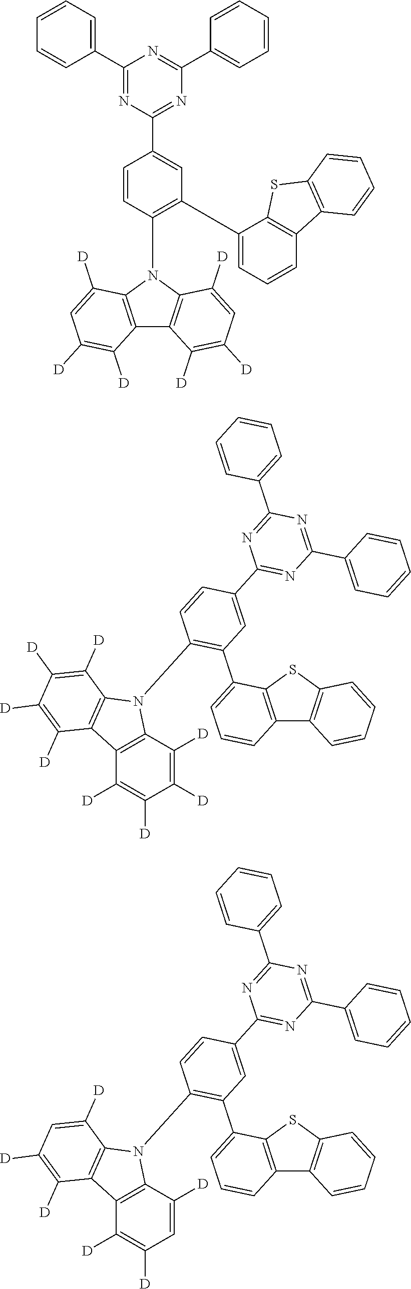 [SMILES: [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1sc1ccccc12 ; [2H]c1c([2H])c([2H])c2c(c1[2H])c1c([2H])c([2H])c([2H])c([2H])c1n2-c1ccc(-c2nc(-c3ccccc3)nc(-c3ccccc3)n2)cc1-c1cccc2c1sc1ccccc12 ; [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1sc1ccccc12]

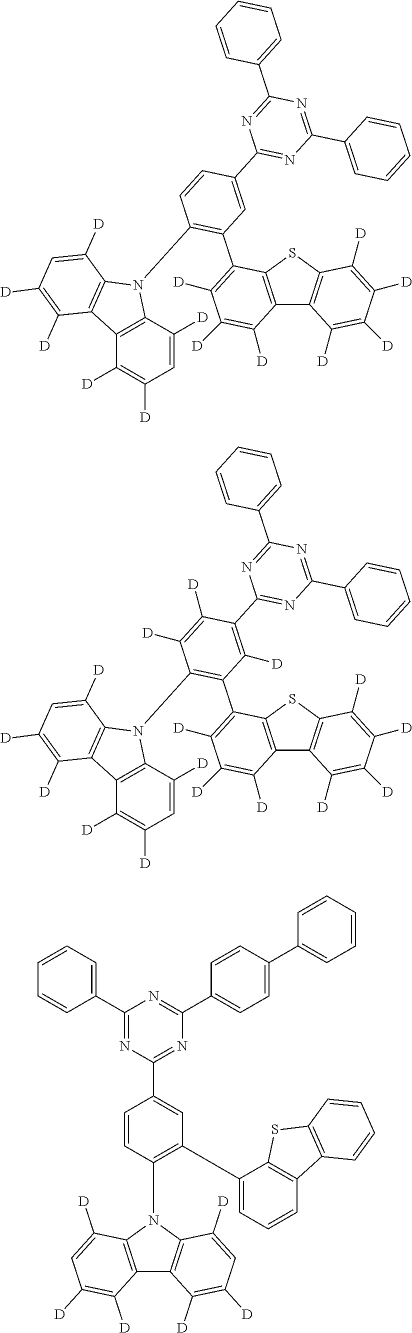 [SMILES: [2H]c1cc([2H])c2c(c1[2H])c1c([2H])c([2H])cc([2H])c1n2-c1ccc(-c2nc(-c3ccccc3)nc(-c3ccccc3)n2)cc1-c1c([2H])c([2H])c([2H])c2c1sc1c([2H])c([2H])c([2H])c([2H])c12 ; [2H]c1cc([2H])c2c(c1[2H])c1c([2H])c([2H])cc([2H])c1n2-c1c([2H])c([2H])c(-c2nc(-c3ccccc3)nc(-c3ccccc3)n2)c([2H])c1-c1c([2H])c([2H])c([2H])c2c1sc1c([2H])c([2H])c([2H])c([2H])c12 ; [2H]c1cc([2H])c2c(c1[2H])c1c([2H])c([2H])cc([2H])c1n2-c1ccc(-c2nc(-c3ccccc3)nc(-c3ccc(-c4ccccc4)cc3)n2)cc1-c1cccc2c1sc1ccccc12]

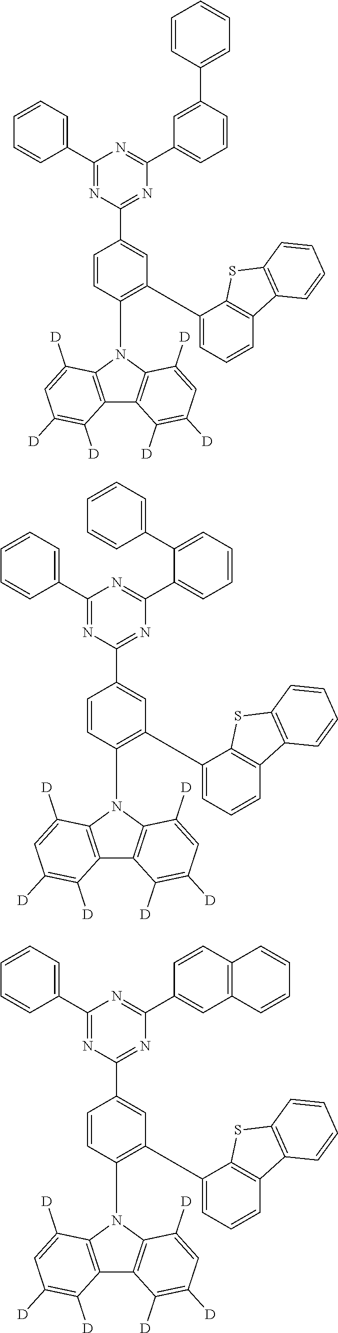

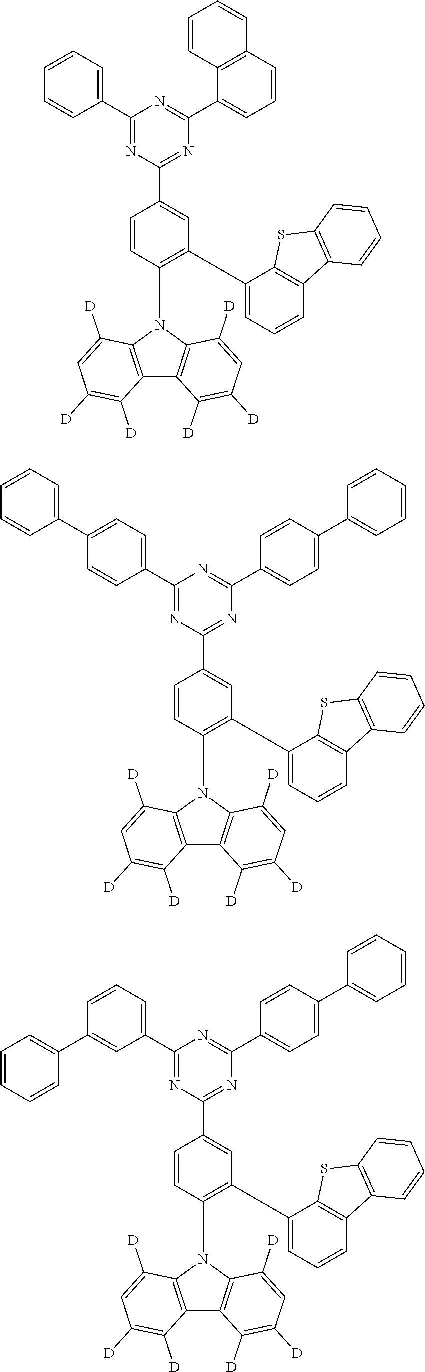 [SMILES: [2H]c1cc([2H])c2c(c1[2H])c1c([2H])c([2H])cc([2H])c1n2-c1ccc(-c2nc(-c3ccccc3)nc(-c3cccc4ccccc34)n2)cc1-c1cccc2c1sc1ccccc12 ; [2H]c1cc([2H])c2c(c1[2H])c1c([2H])c([2H])cc([2H])c1n2-c1ccc(-c2nc(-c3ccc(-c4ccccc4)cc3)nc(-c3ccc(-c4ccccc4)cc3)n2)cc1-c1cccc2c1sc1ccccc12 ; [2H]c1cc([2H])c2c(c1[2H])c1c([2H])c([2H])cc([2H])c1n2-c1ccc(-c2nc(-c3ccc(-c4ccccc4)cc3)nc(-c3cccc(-c4ccccc4)c3)n2)cc1-c1cccc2c1sc1ccccc12]

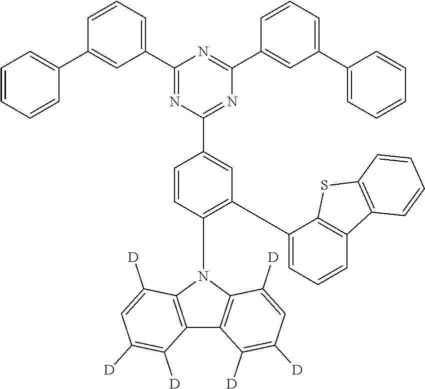 [SMILES: [2H]c1cc([2H])c2c(c1[2H])c1c([2H])c([2H])cc([2H])c1n2-c1ccc(-c2nc(-c3cccc(-c4ccccc4)c3)nc(-c3cccc(-c4ccccc4)c3)n2)cc1-c1cccc2c1sc1ccccc12]

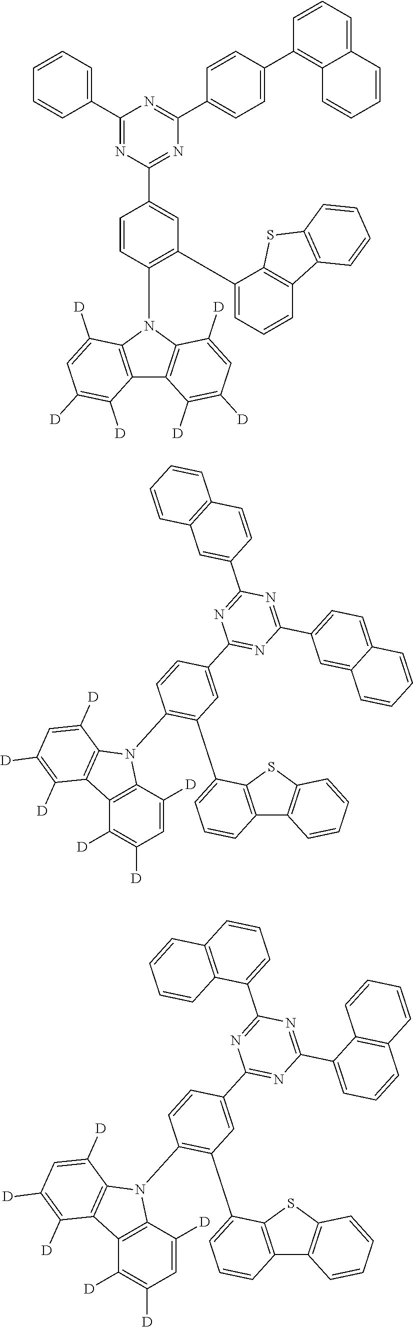

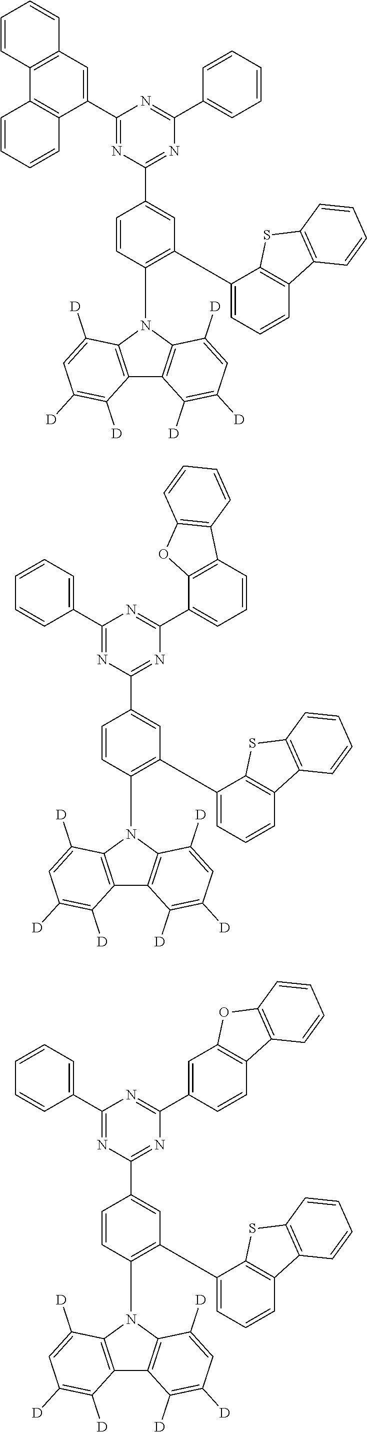

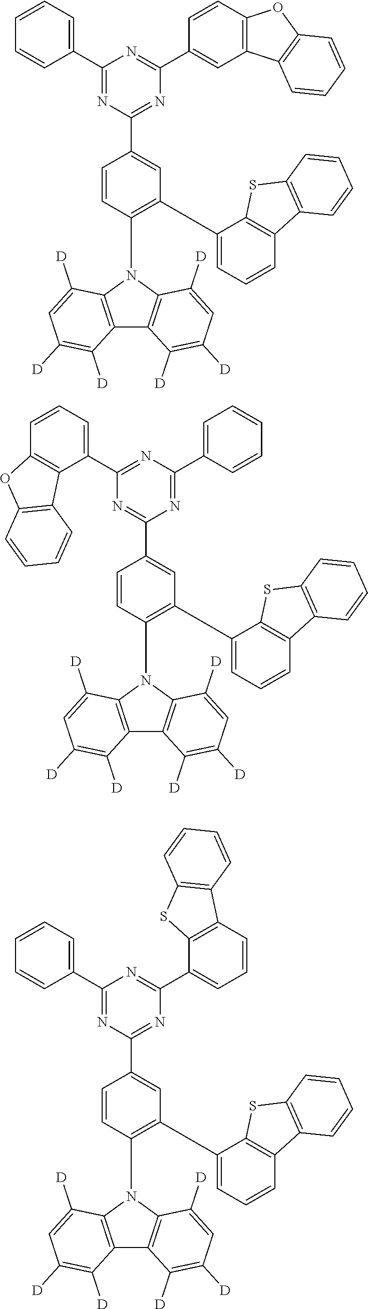

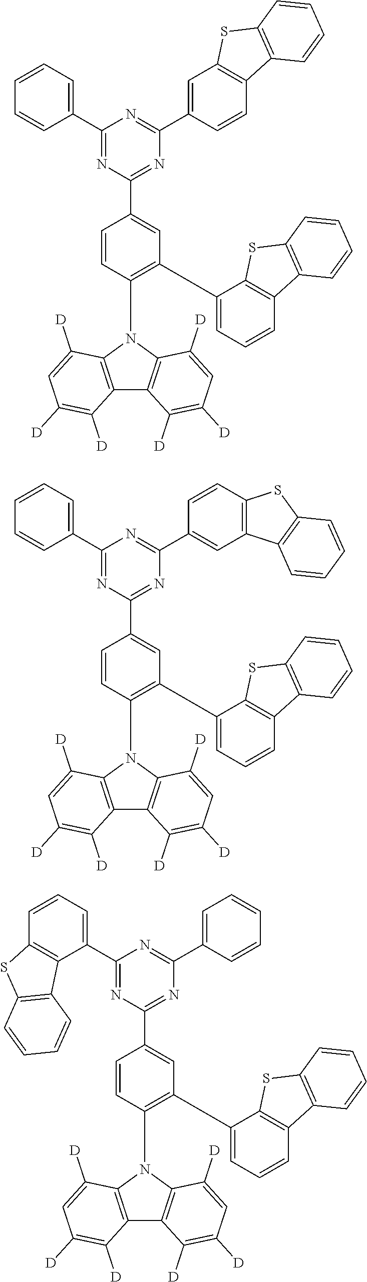

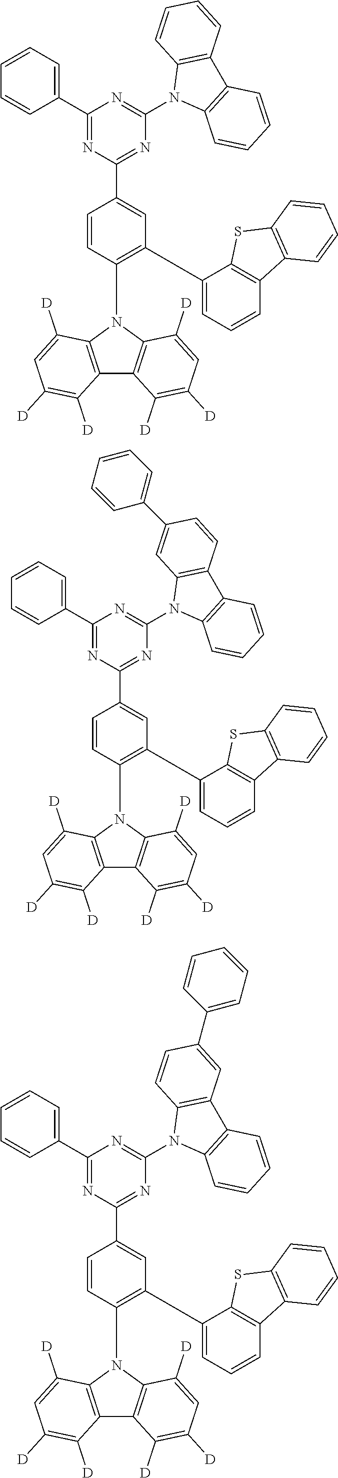

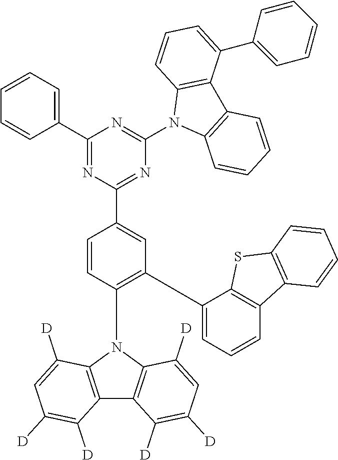 [SMILES: [2H]c1cc([2H])c2c(c1[2H])c1c([2H])c([2H])cc([2H])c1n2-c1ccc(-c2nc(-c3ccccc3)nc(-n3c4ccccc4c4c(-c5ccccc5)cccc43)n2)cc1-c1cccc2c1sc1ccccc12]

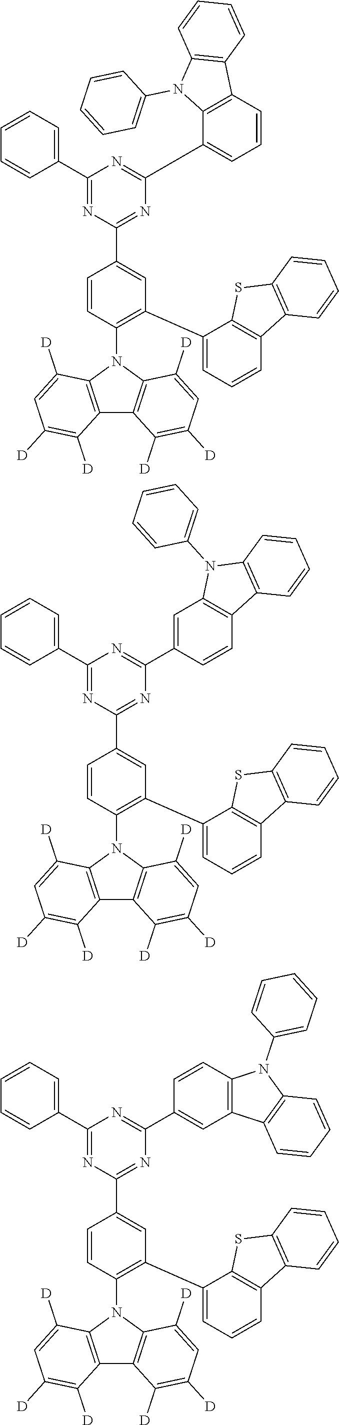

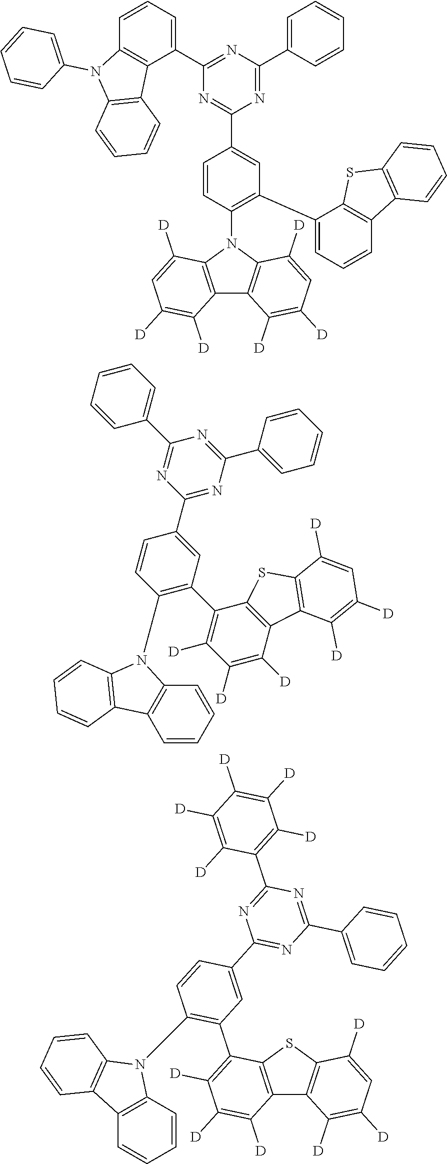

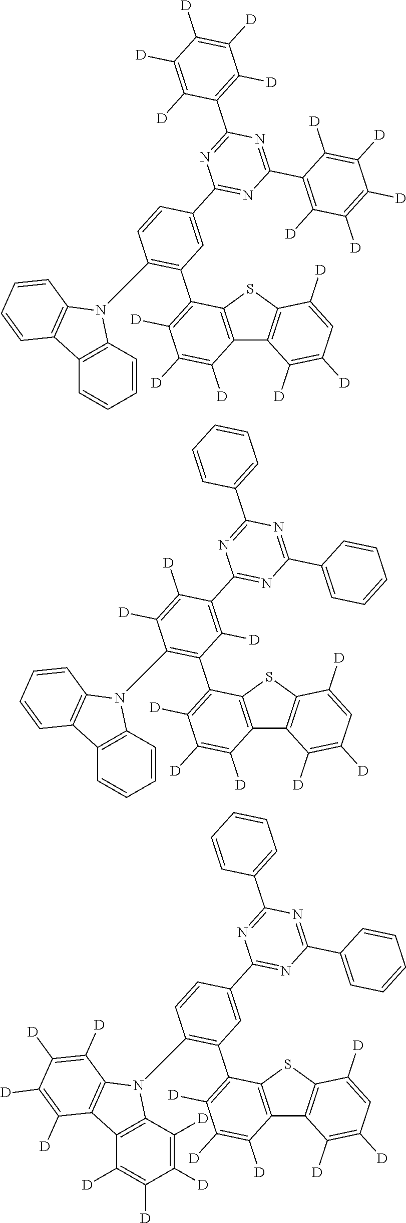

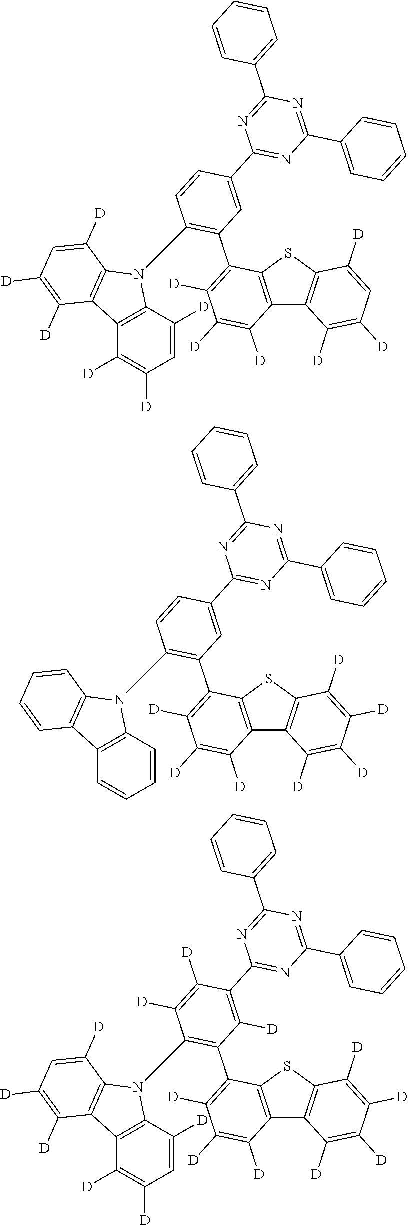

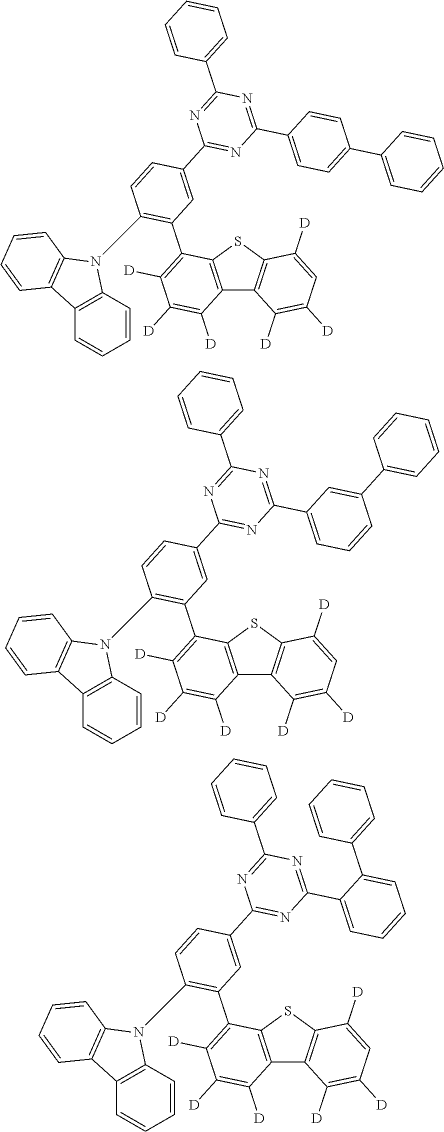

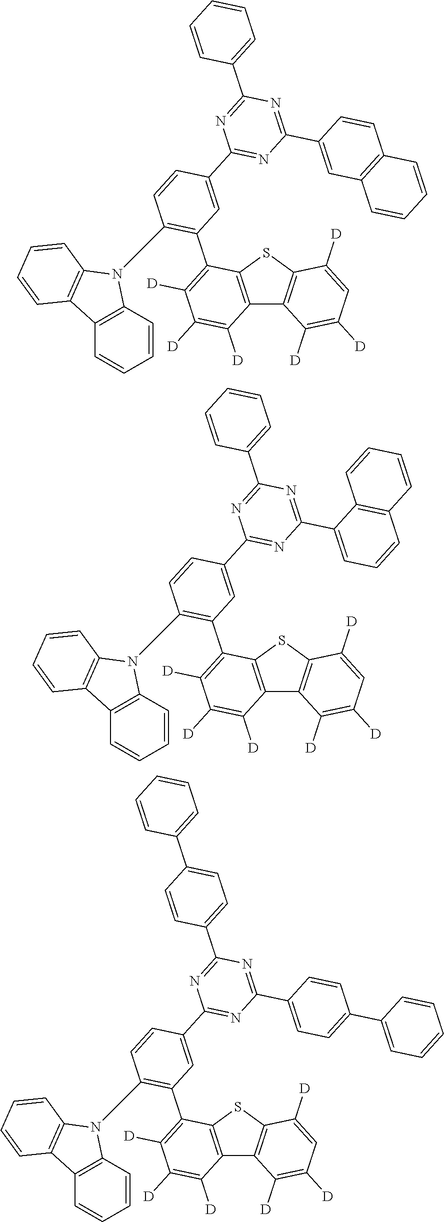

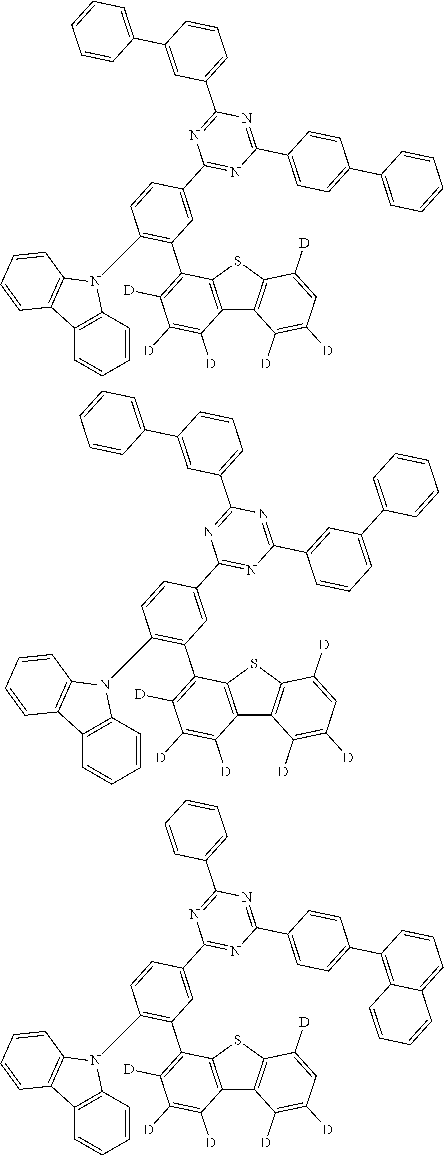

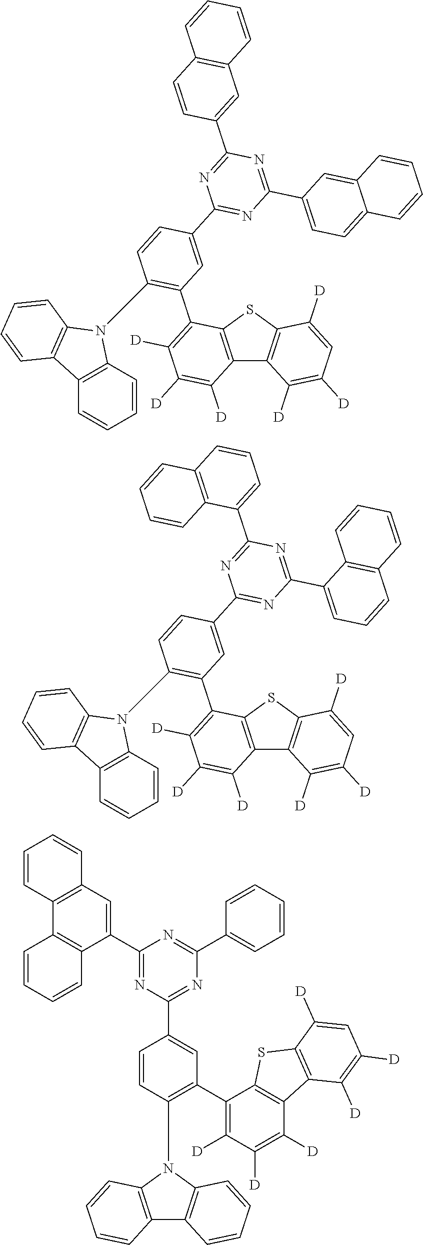

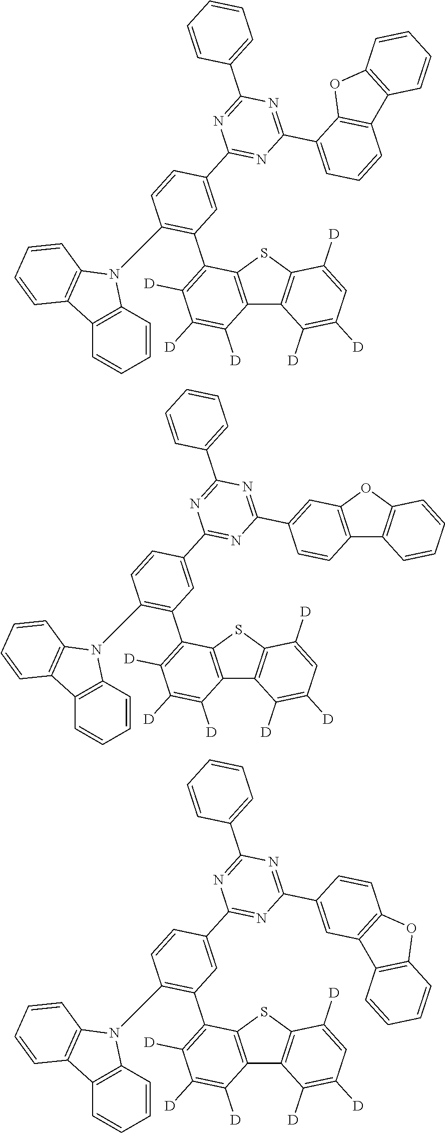

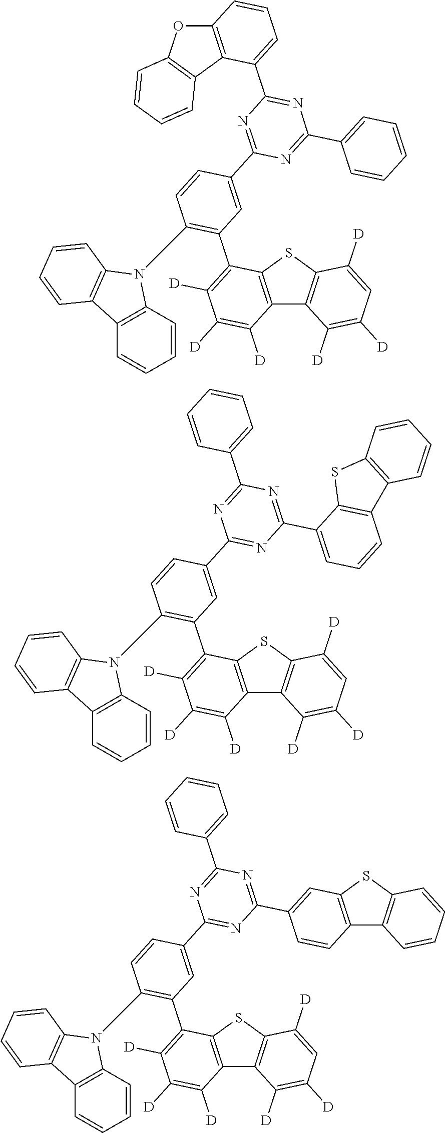

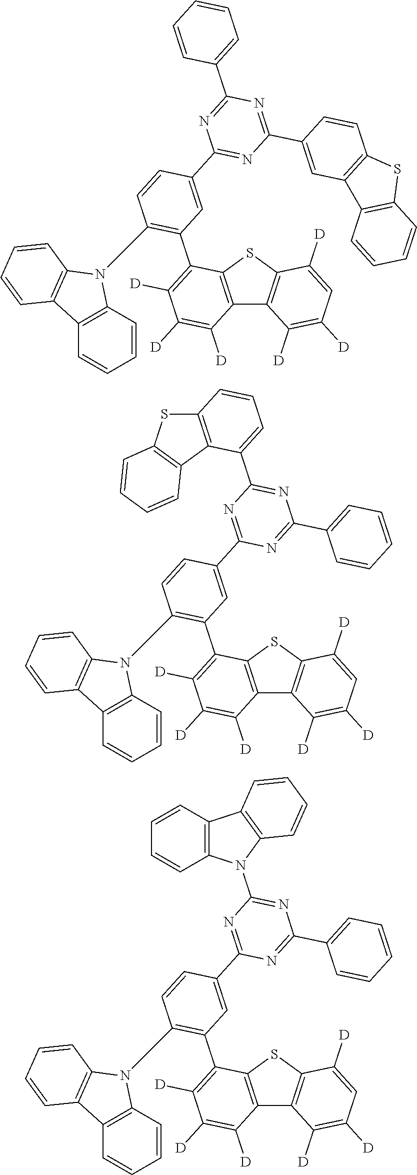

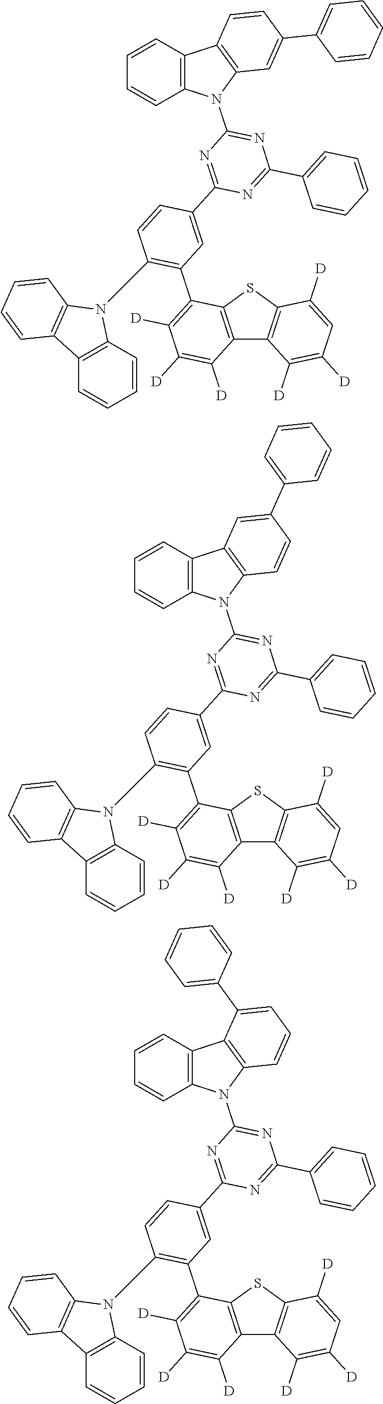

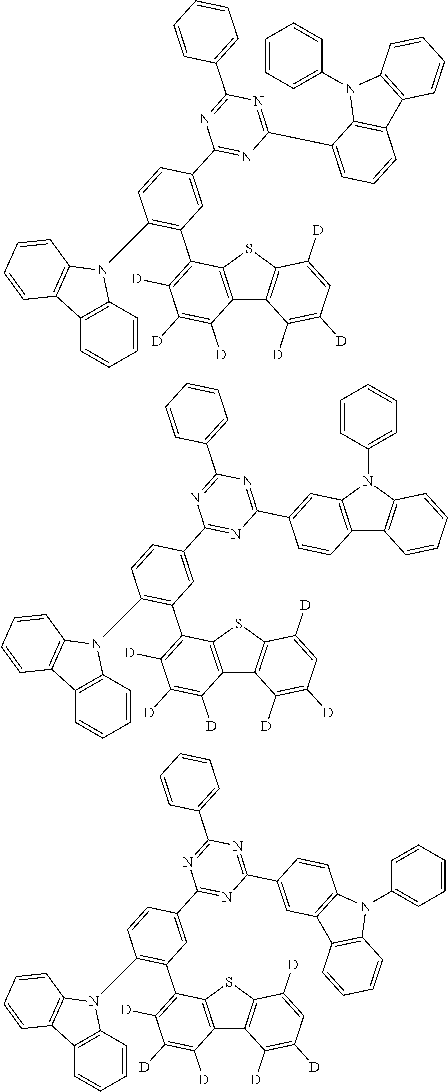

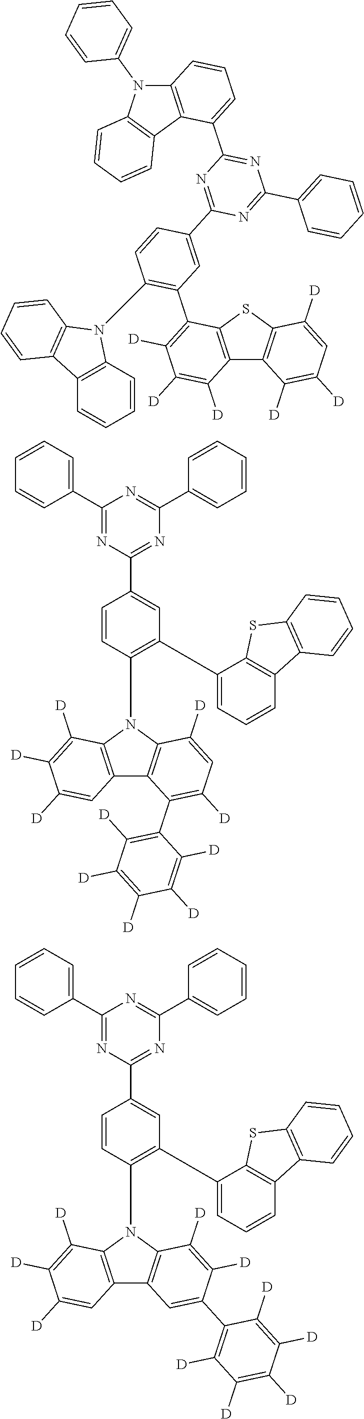

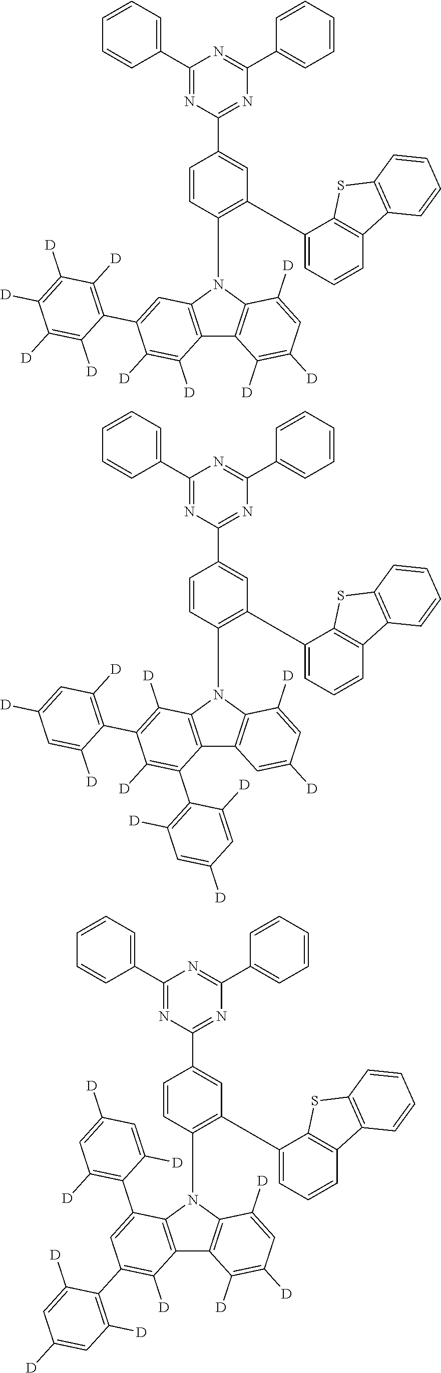

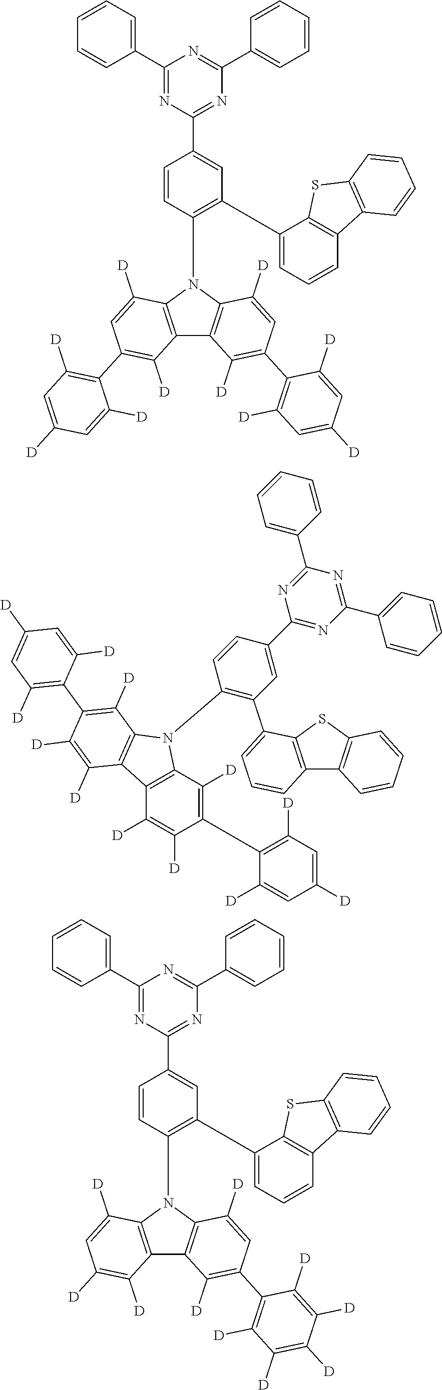

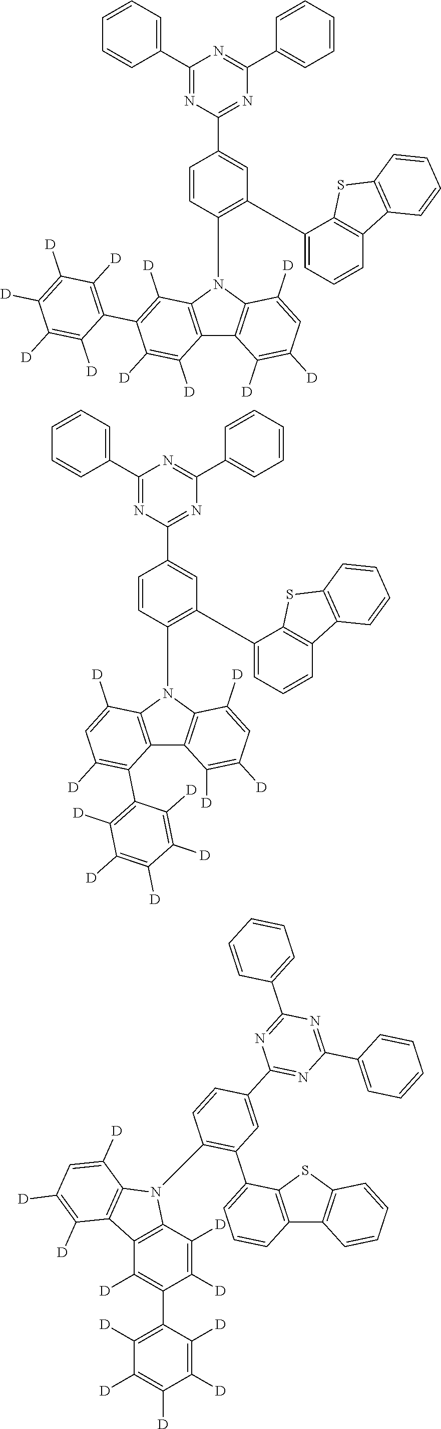

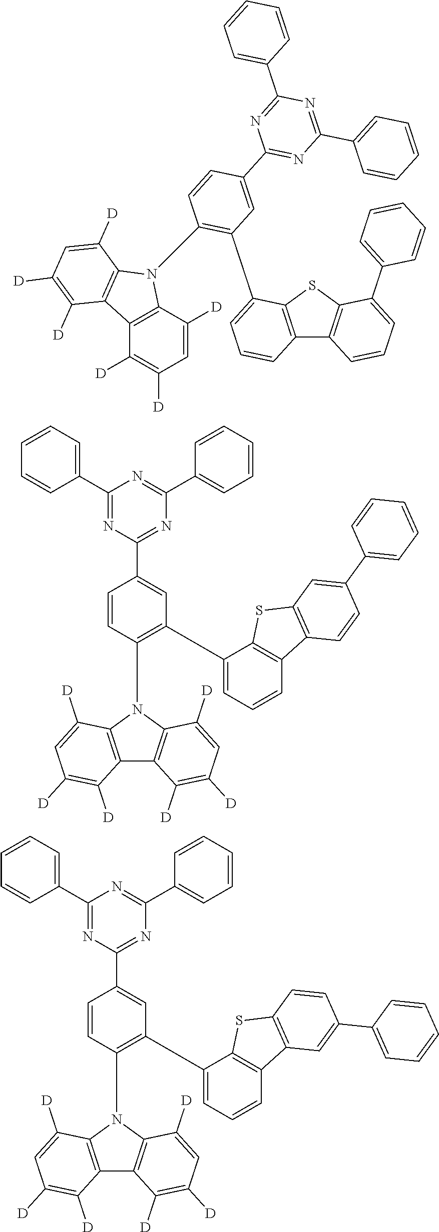

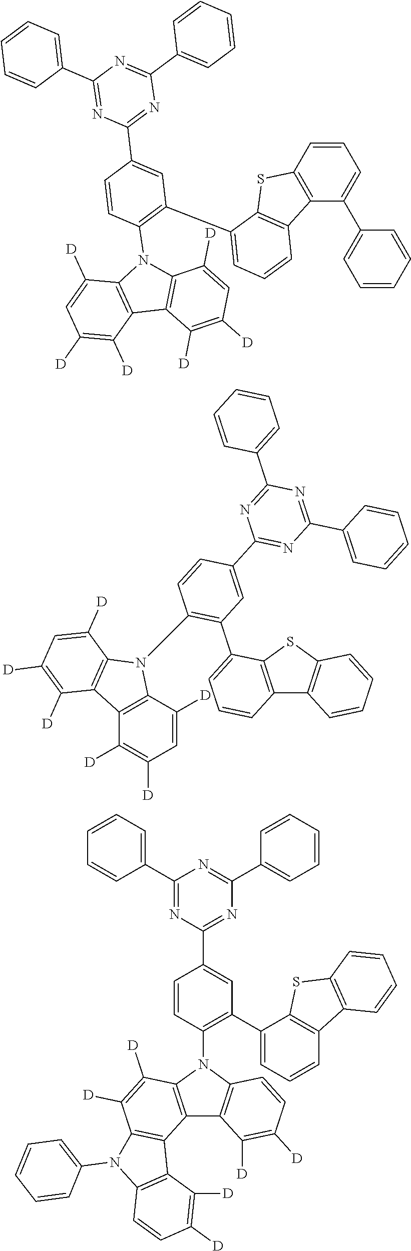

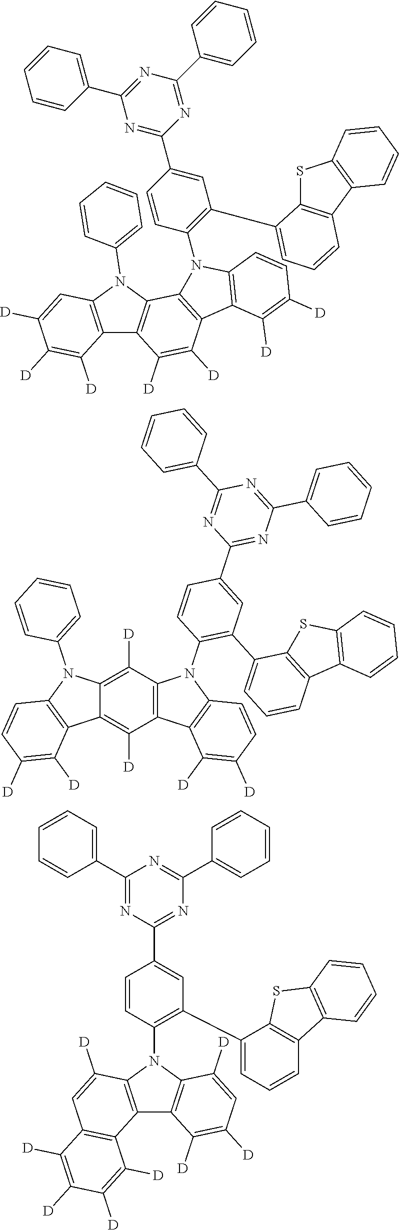

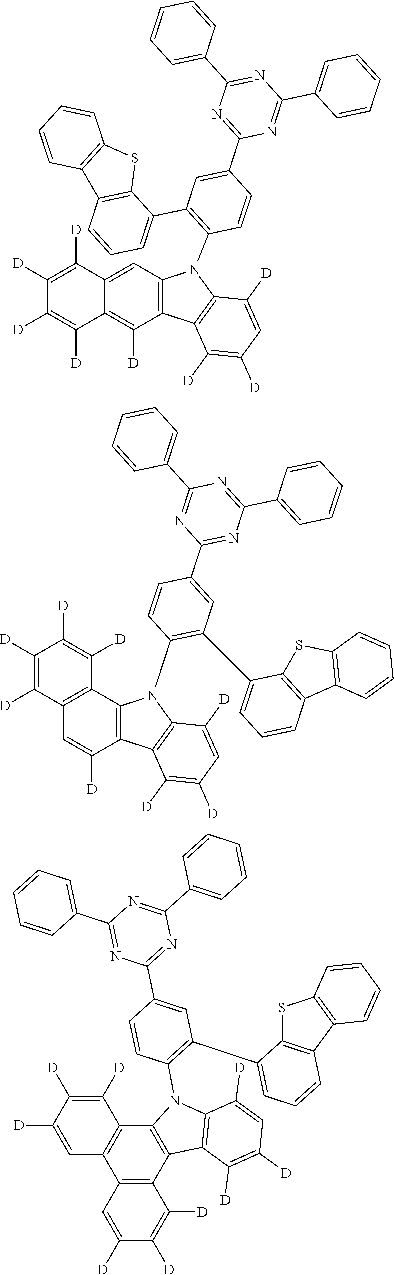

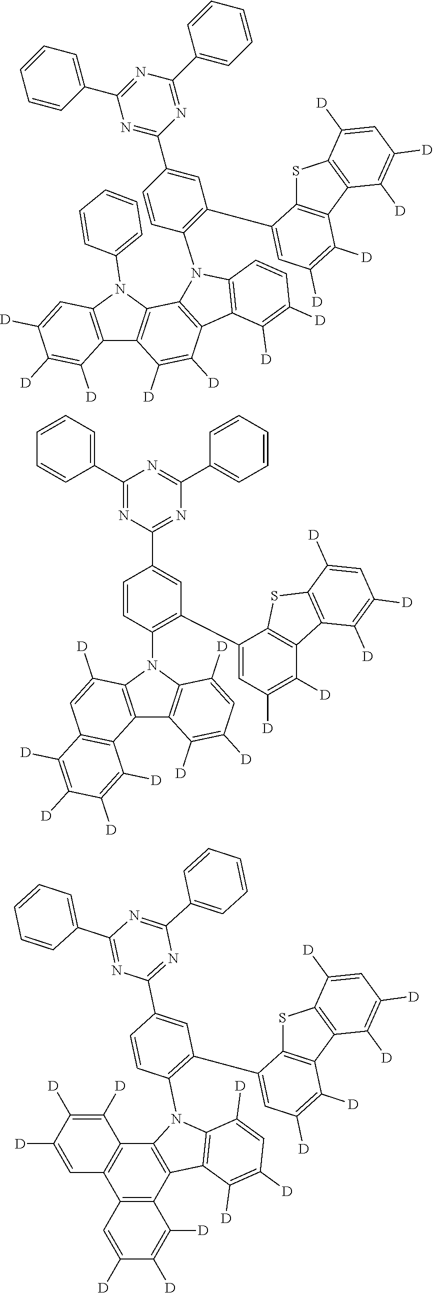

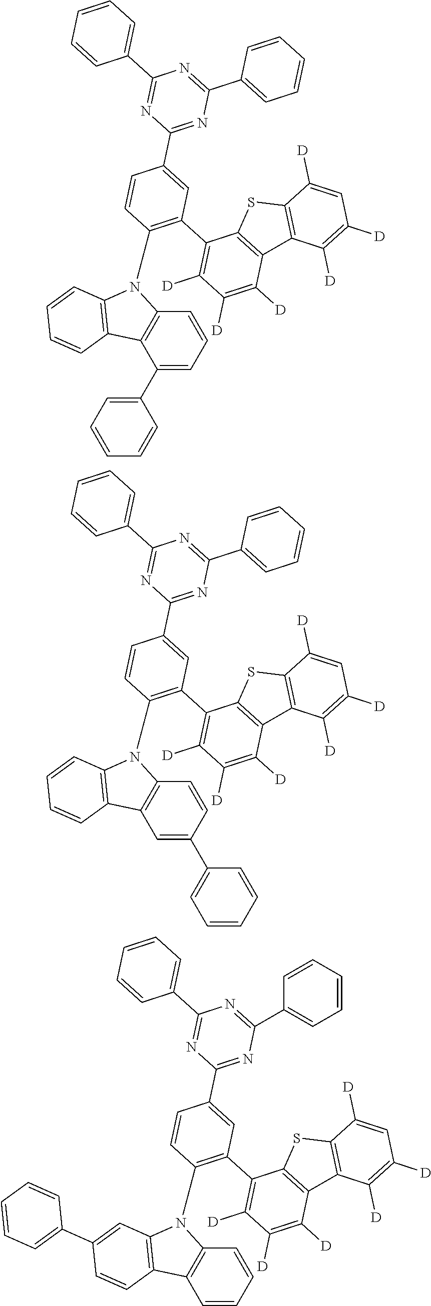

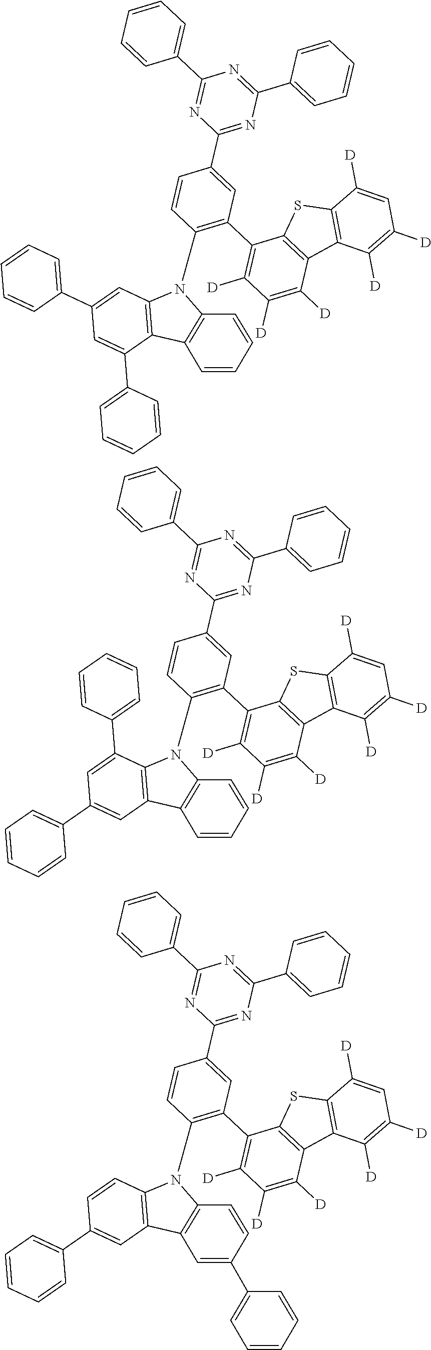

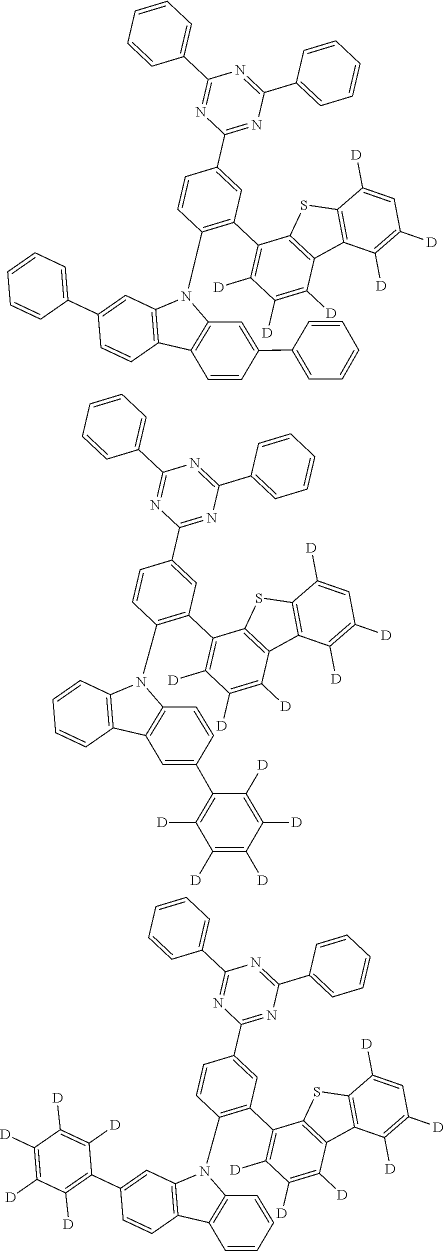

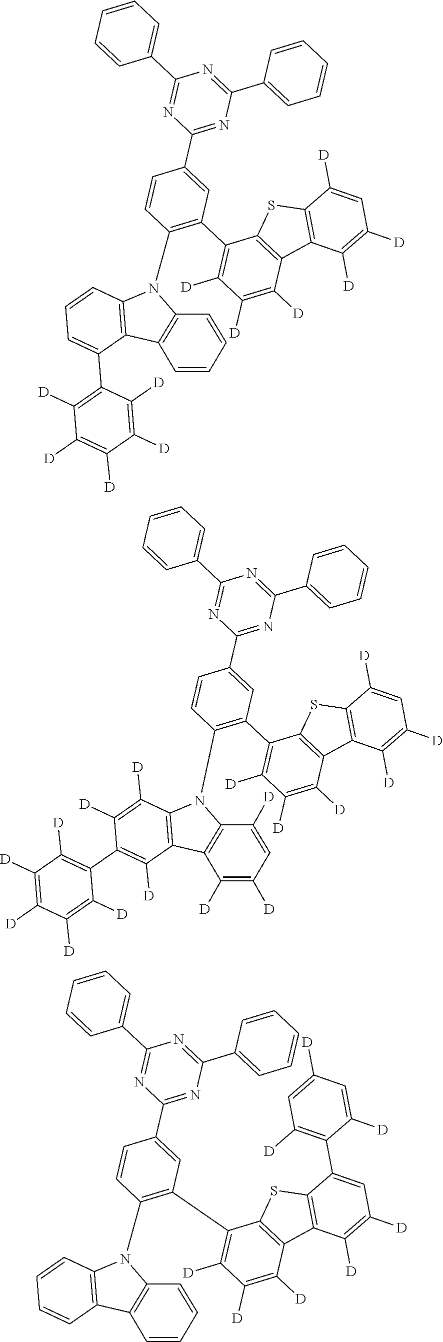

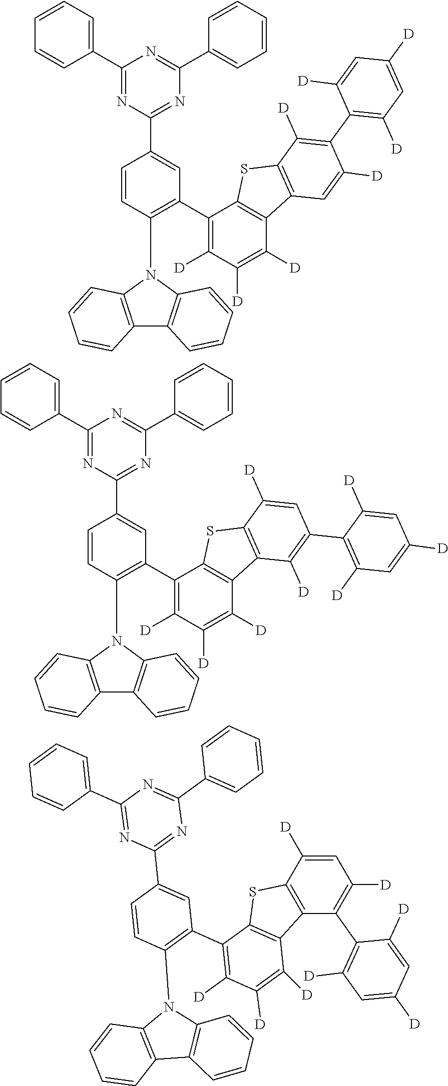

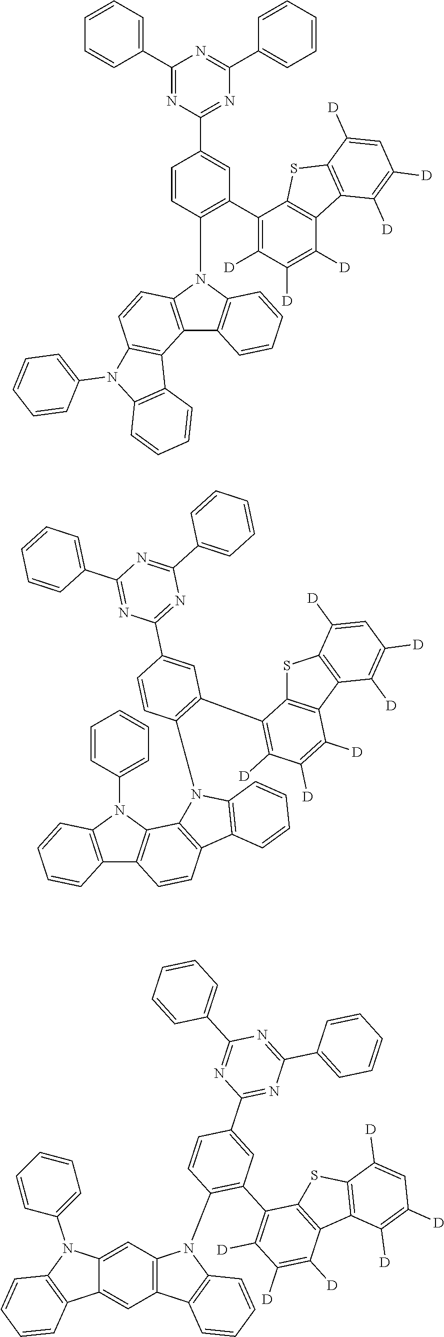 [SMILES: [2H]c1cc([2H])c2sc3c(-c4cc(-c5nc(-c6ccccc6)nc(-c6ccccc6)n5)ccc4-n4c5ccccc5c5c6c7ccccc7n(-c7ccccc7)c6ccc54)c([2H])c([2H])c([2H])c3c2c1[2H] ; [2H]c1cc([2H])c2sc3c(-c4cc(-c5nc(-c6ccccc6)nc(-c6ccccc6)n5)ccc4-n4c5ccccc5c5ccc6c7ccccc7n(-c7ccccc7)c6c54)c([2H])c([2H])c([2H])c3c2c1[2H] ; [2H]c1cc([2H])c2sc3c(-c4cc(-c5nc(-c6ccccc6)nc(-c6ccccc6)n5)ccc4-n4c5ccccc5c5cc6c7ccccc7n(-c7ccccc7)c6cc54)c([2H])c([2H])c([2H])c3c2c1[2H]]

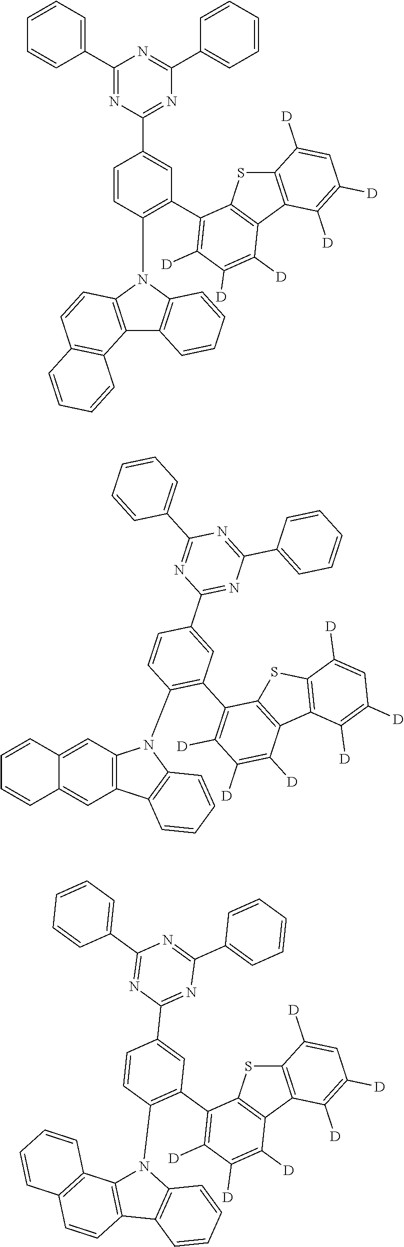 [SMILES: [2H]c1cc([2H])c2sc3c(-c4cc(-c5nc(-c6ccccc6)nc(-c6ccccc6)n5)ccc4-n4c5ccccc5c5c6ccccc6ccc54)c([2H])c([2H])c([2H])c3c2c1[2H] ; [2H]c1cc([2H])c2sc3c(-c4cc(-c5nc(-c6ccccc6)nc(-c6ccccc6)n5)ccc4-n4c5ccccc5c5cc6ccccc6cc54)c([2H])c([2H])c([2H])c3c2c1[2H] ; [2H]c1cc([2H])c2sc3c(-c4cc(-c5nc(-c6ccccc6)nc(-c6ccccc6)n5)ccc4-n4c5ccccc5c5ccc6ccccc6c54)c([2H])c([2H])c([2H])c3c2c1[2H]]

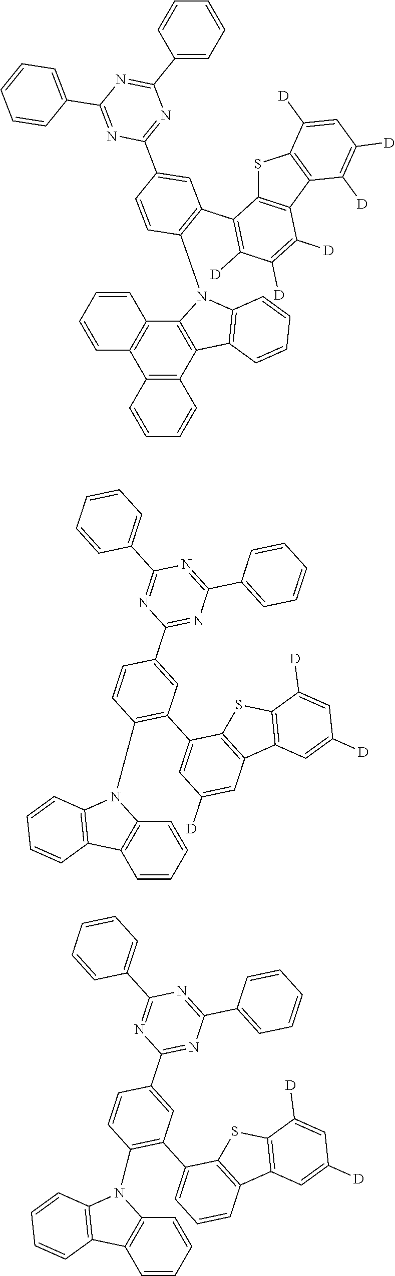

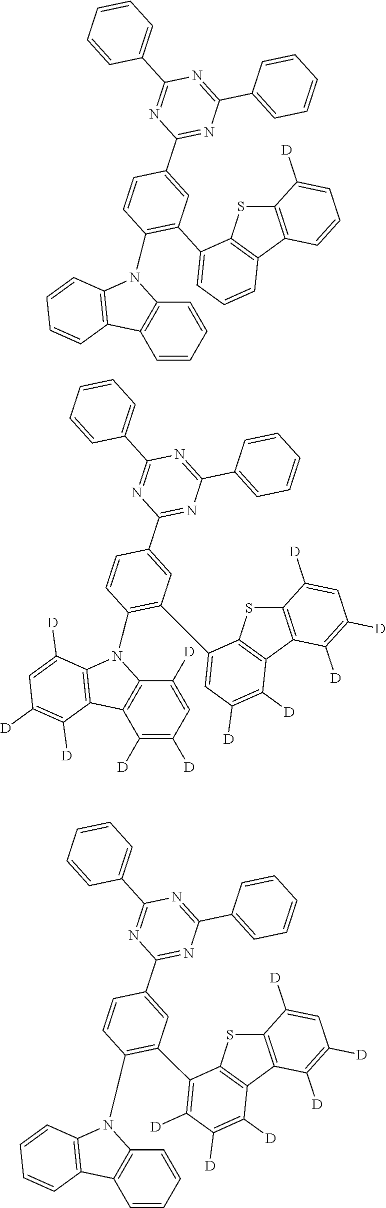

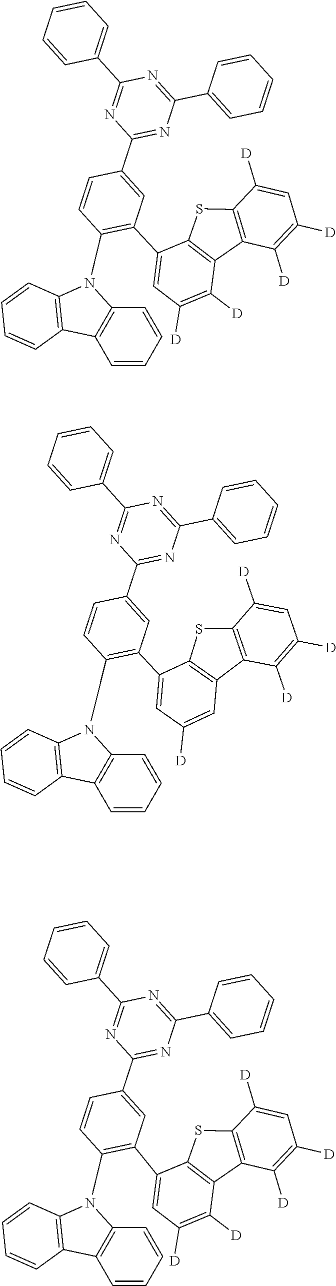 [SMILES: [2H]c1cc([2H])c2sc3c(-c4cc(-c5nc(-c6ccccc6)nc(-c6ccccc6)n5)ccc4-n4c5ccccc5c5ccccc54)cc([2H])c([2H])c3c2c1[2H] ; [2H]c1cc(-c2cc(-c3nc(-c4ccccc4)nc(-c4ccccc4)n3)ccc2-n2c3ccccc3c3ccccc32)c2sc3c([2H])cc([2H])c([2H])c3c2c1 ; [2H]c1cc([2H])c2sc3c(-c4cc(-c5nc(-c6ccccc6)nc(-c6ccccc6)n5)ccc4-n4c5ccccc5c5ccccc54)cc([2H])c([2H])c3c2c1[2H]]

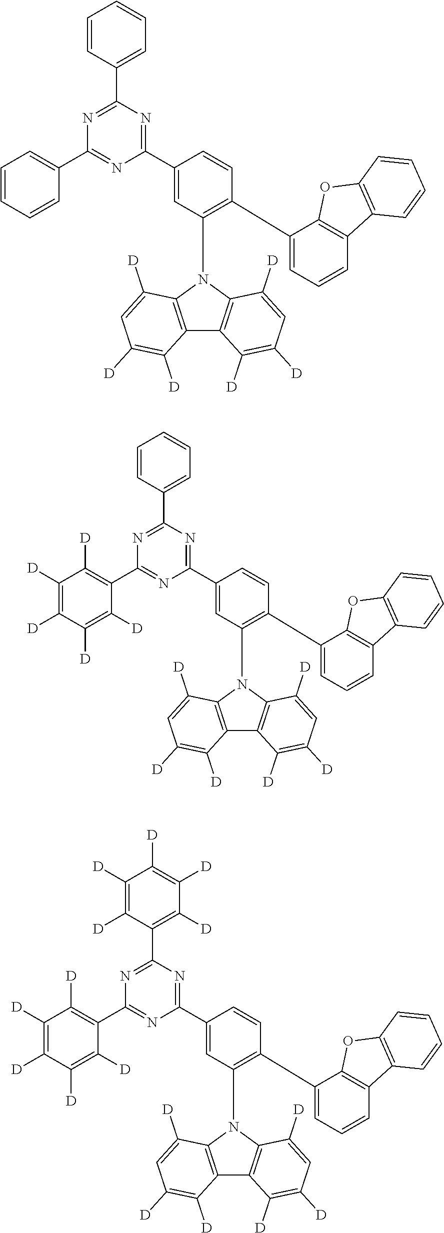 [SMILES: [2H]c1cc([2H])c2c(c1[2H])c1c([2H])c([2H])cc([2H])c1n2-c1cc(-c2nc(-c3ccccc3)nc(-c3ccccc3)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3c([2H])c([2H])c([2H])c([2H])c3[2H])n2)ccc1-c1cccc2c1oc1ccccc12 ; [2H]c1cc([2H])c2c(c1[2H])c1c([2H])c([2H])cc([2H])c1n2-c1cc(-c2nc(-c3c([2H])c([2H])c([2H])c([2H])c3[2H])nc(-c3c([2H])c([2H])c([2H])c([2H])c3[2H])n2)ccc1-c1cccc2c1oc1ccccc12]

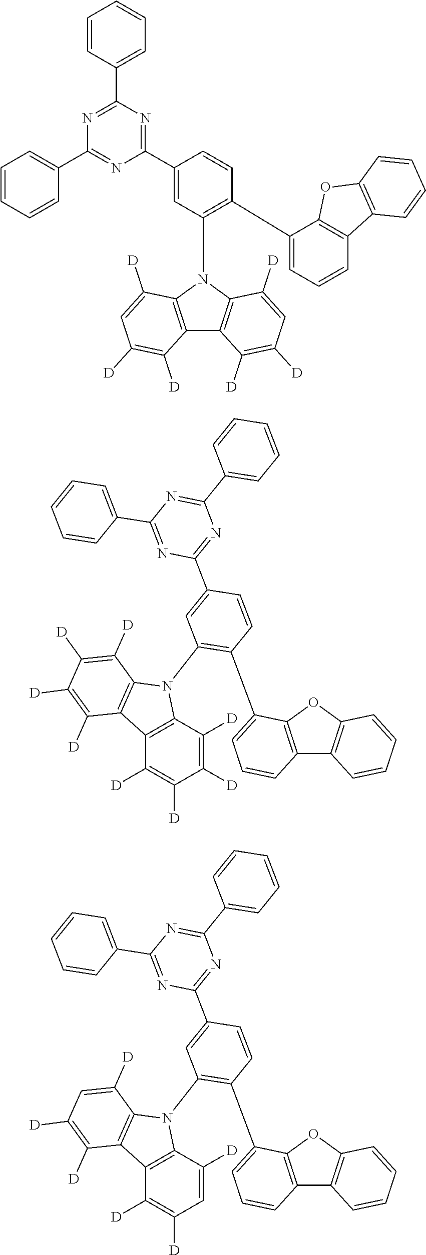

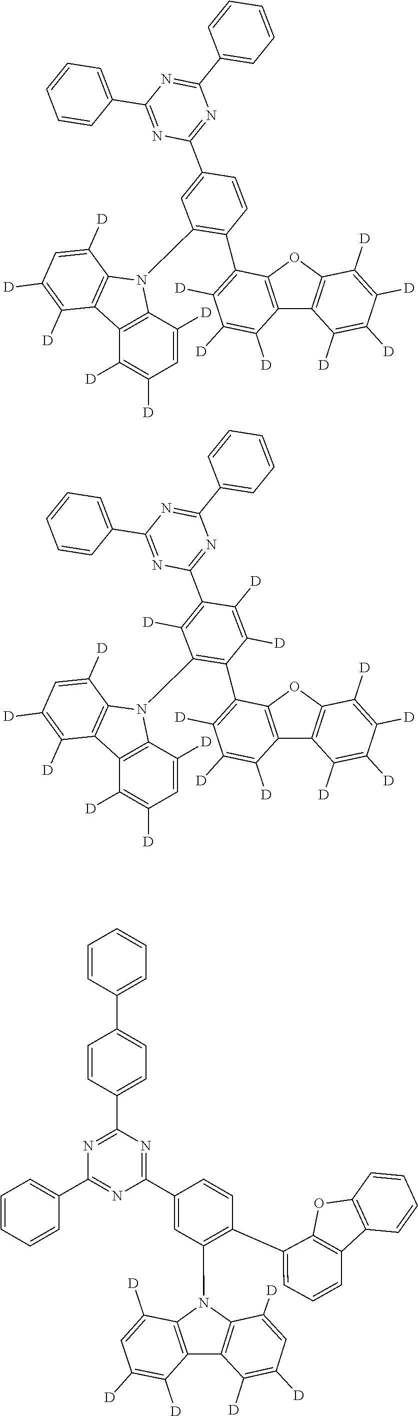 [SMILES: [2H]c1cc([2H])c2c(c1[2H])c1c([2H])c([2H])cc([2H])c1n2-c1cc(-c2nc(-c3ccccc3)nc(-c3ccccc3)n2)ccc1-c1c([2H])c([2H])c([2H])c2c1oc1c([2H])c([2H])c([2H])c([2H])c12 ; [2H]c1cc([2H])c2c(c1[2H])c1c([2H])c([2H])cc([2H])c1n2-c1c([2H])c(-c2nc(-c3ccccc3)nc(-c3ccccc3)n2)c([2H])c([2H])c1-c1c([2H])c([2H])c([2H])c2c1oc1c([2H])c([2H])c([2H])c([2H])c12 ; [2H]c1cc([2H])c2c(c1[2H])c1c([2H])c([2H])cc([2H])c1n2-c1cc(-c2nc(-c3ccccc3)nc(-c3ccc(-c4ccccc4)cc3)n2)ccc1-c1cccc2c1oc1ccccc12]

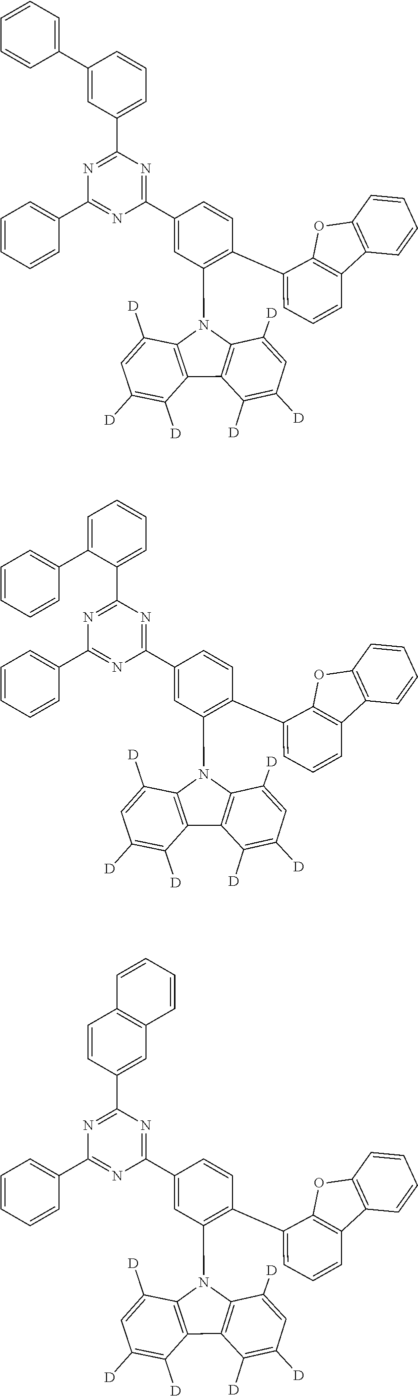 [SMILES: [2H]c1cc([2H])c2c(c1[2H])c1c([2H])c([2H])cc([2H])c1n2-c1cc(-c2nc(-c3ccccc3)nc(-c3cccc(-c4ccccc4)c3)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3ccccc3-c3ccccc3)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3ccc4ccccc4c3)n2)ccc1-c1cccc2c1oc1ccccc12]

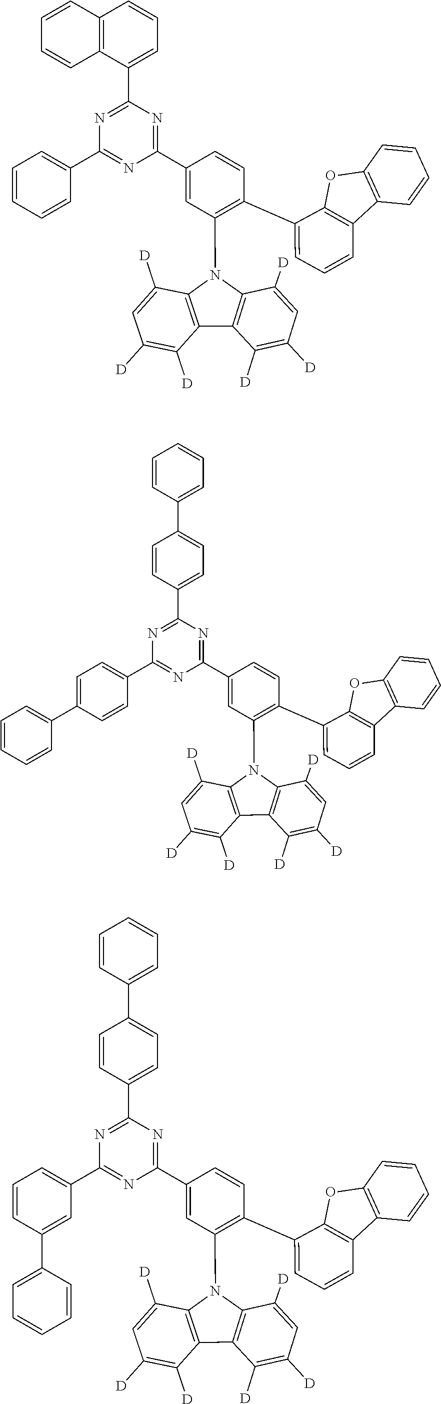 [SMILES: [2H]c1cc([2H])c2c(c1[2H])c1c([2H])c([2H])cc([2H])c1n2-c1cc(-c2nc(-c3ccccc3)nc(-c3cccc4ccccc34)n2)ccc1-c1cccc2c1oc1ccccc12 ; [2H]c1cc([2H])c2c(c1[2H])c1c([2H])c([2H])cc([2H])c1n2-c1cc(-c2nc(-c3ccc(-c4ccccc4)cc3)nc(-c3ccc(-c4ccccc4)cc3)n2)ccc1-c1cccc2c1oc1ccccc12 ; [2H]c1cc([2H])c2c(c1[2H])c1c([2H])c([2H])cc([2H])c1n2-c1cc(-c2nc(-c3ccc(-c4ccccc4)cc3)nc(-c3cccc(-c4ccccc4)c3)n2)ccc1-c1cccc2c1oc1ccccc12]

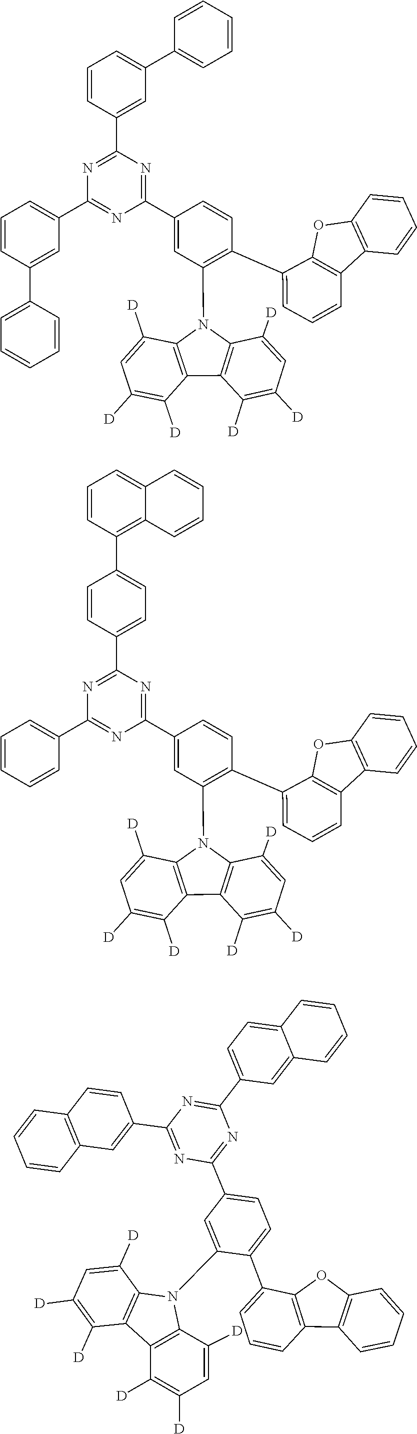

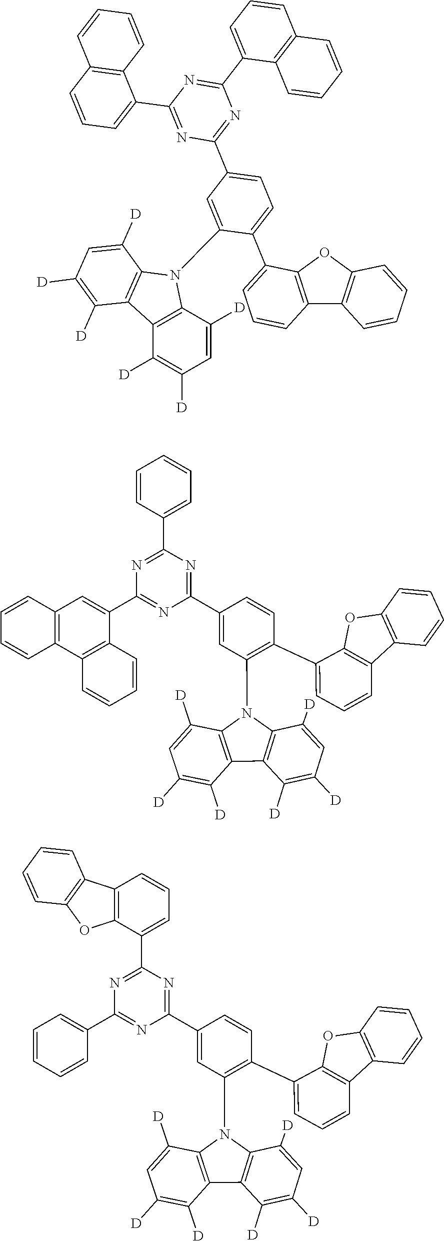 [SMILES: [2H]c1cc([2H])c2c(c1[2H])c1c([2H])c([2H])cc([2H])c1n2-c1cc(-c2nc(-c3cccc4ccccc34)nc(-c3cccc4ccccc34)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3cc4ccccc4c4ccccc34)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3cccc4c3oc3ccccc34)n2)ccc1-c1cccc2c1oc1ccccc12]

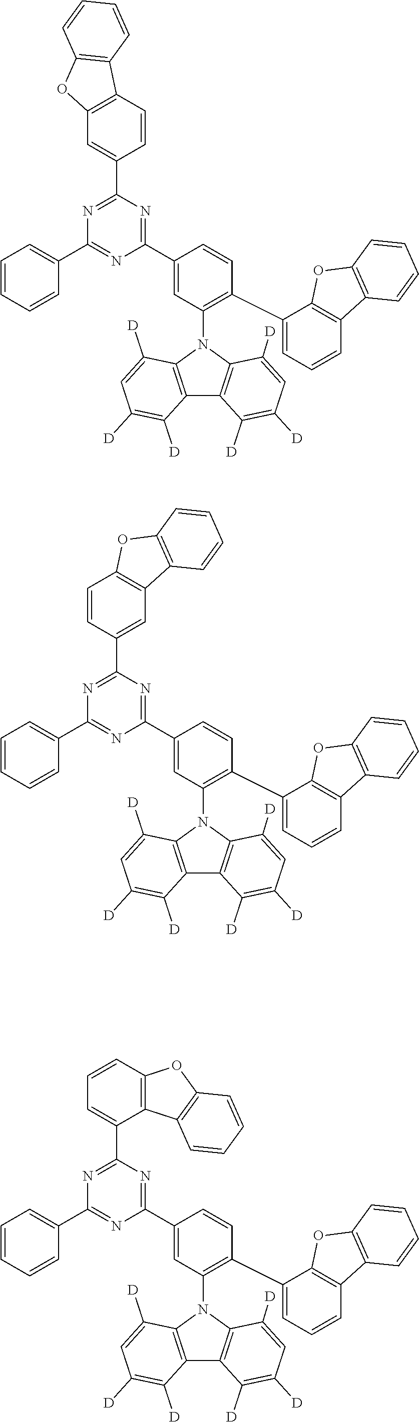 [SMILES: [2H]c1cc([2H])c2c(c1[2H])c1c([2H])c([2H])cc([2H])c1n2-c1cc(-c2nc(-c3ccccc3)nc(-c3ccc4c(c3)oc3ccccc34)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3ccc4oc5ccccc5c4c3)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3cccc4oc5ccccc5c34)n2)ccc1-c1cccc2c1oc1ccccc12]

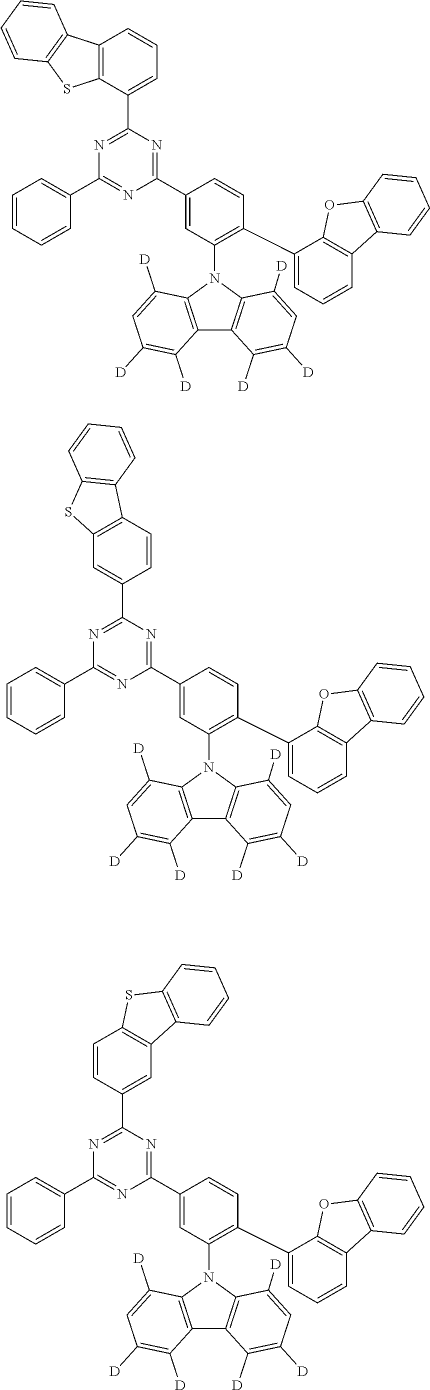 [SMILES: [2H]c1cc([2H])c2c(c1[2H])c1c([2H])c([2H])cc([2H])c1n2-c1cc(-c2nc(-c3ccccc3)nc(-c3cccc4c3sc3ccccc34)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3ccc4c(c3)sc3ccccc34)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3ccc4sc5ccccc5c4c3)n2)ccc1-c1cccc2c1oc1ccccc12]

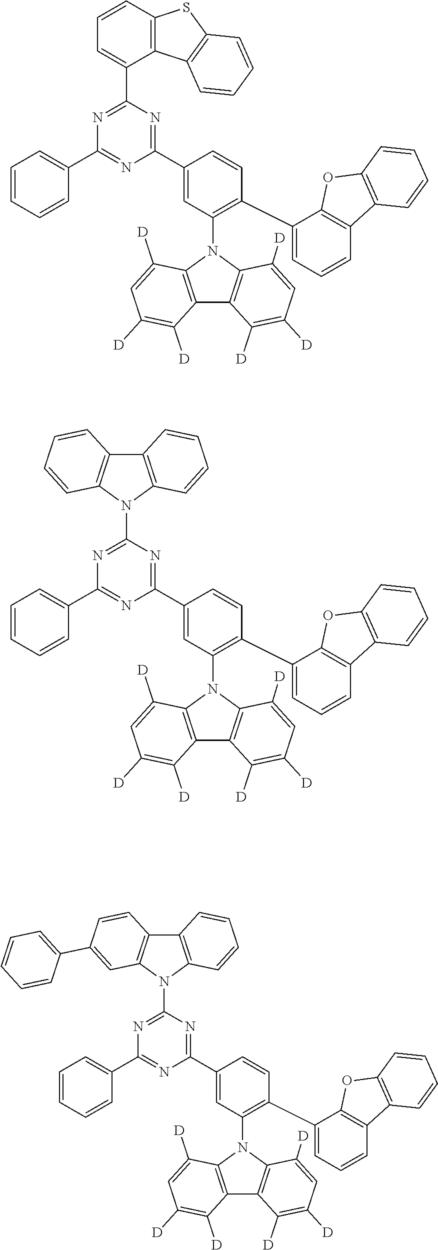 [SMILES: [2H]c1cc([2H])c2c(c1[2H])c1c([2H])c([2H])cc([2H])c1n2-c1cc(-c2nc(-c3ccccc3)nc(-c3cccc4sc5ccccc5c34)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-n3c4ccccc4c4ccccc43)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-n3c4ccccc4c4ccc(-c5ccccc5)cc43)n2)ccc1-c1cccc2c1oc1ccccc12]

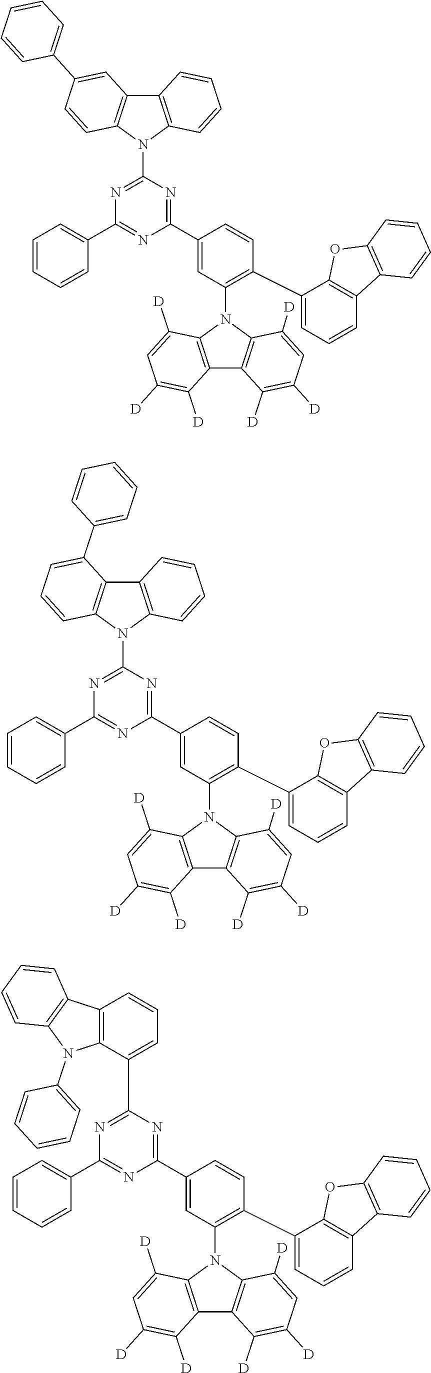 [SMILES: [2H]c1cc([2H])c2c(c1[2H])c1c([2H])c([2H])cc([2H])c1n2-c1cc(-c2nc(-c3ccccc3)nc(-n3c4ccccc4c4cc(-c5ccccc5)ccc43)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-n3c4ccccc4c4c(-c5ccccc5)cccc43)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3cccc4c5ccccc5n(-c5ccccc5)c34)n2)ccc1-c1cccc2c1oc1ccccc12]

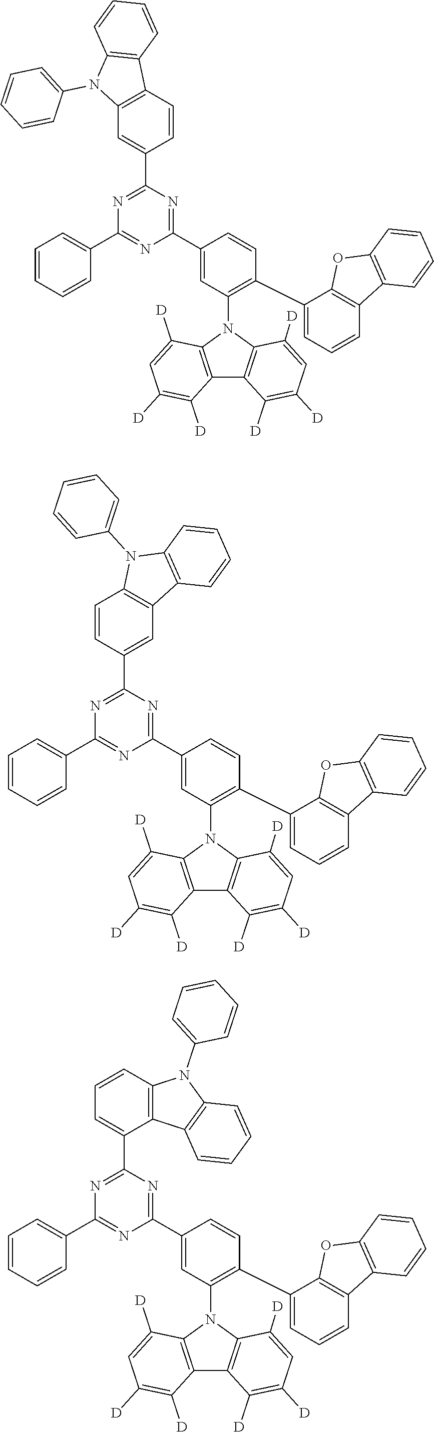

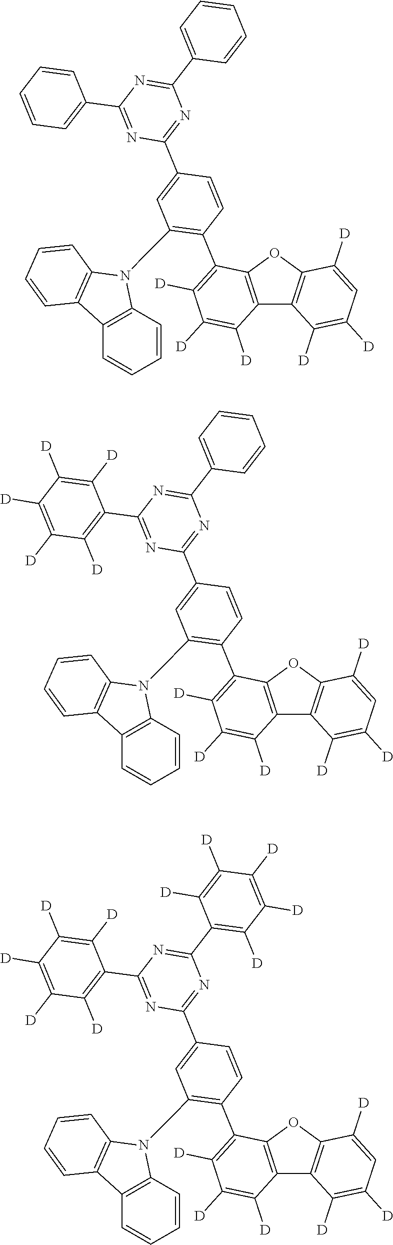 [SMILES: [2H]c1cc([2H])c2oc3c(-c4ccc(-c5nc(-c6ccccc6)nc(-c6ccccc6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6c([2H])c([2H])c([2H])c([2H])c6[2H])n5)cc4-n4c5ccccc5c5ccccc54)c([2H])c([2H])c([2H])c3c2c1[2H] ; [2H]c1cc([2H])c2oc3c(-c4ccc(-c5nc(-c6c([2H])c([2H])c([2H])c([2H])c6[2H])nc(-c6c([2H])c([2H])c([2H])c([2H])c6[2H])n5)cc4-n4c5ccccc5c5ccccc54)c([2H])c([2H])c([2H])c3c2c1[2H]]

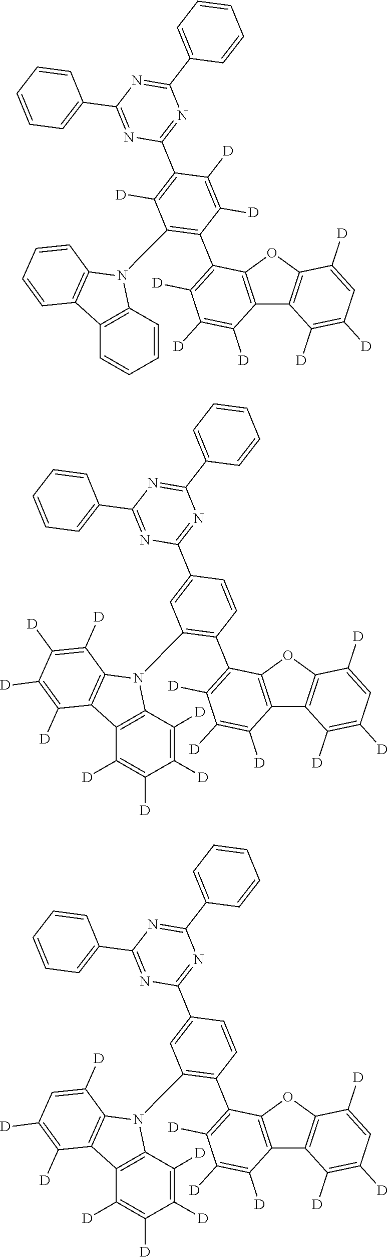 [SMILES: [2H]c1cc([2H])c2oc3c(-c4c([2H])c([2H])c(-c5nc(-c6ccccc6)nc(-c6ccccc6)n5)c([2H])c4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccccc6)n5)cc4-n4c5c([2H])c([2H])c([2H])c([2H])c5c5c([2H])c([2H])c([2H])c([2H])c54)c([2H])c([2H])c([2H])c3c2c1[2H] ; [2H]c1cc([2H])c2oc3c(-c4ccc(-c5nc(-c6ccccc6)nc(-c6ccccc6)n5)cc4-n4c5c([2H])cc([2H])c([2H])c5c5c([2H])c([2H])c([2H])c([2H])c54)c([2H])c([2H])c([2H])c3c2c1[2H]]

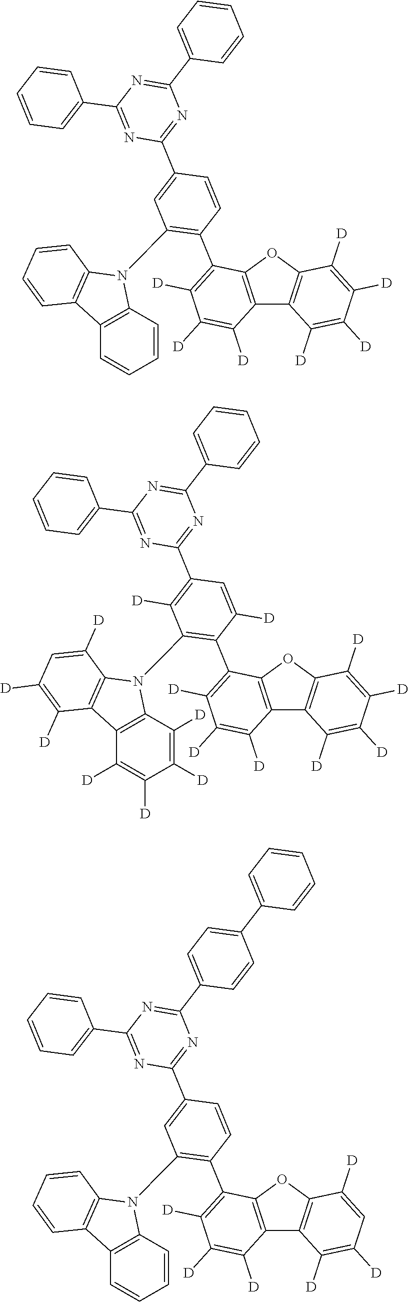 [SMILES: [2H]c1c([2H])c([2H])c2c(oc3c(-c4ccc(-c5nc(-c6ccccc6)nc(-c6ccccc6)n5)cc4-n4c5ccccc5c5ccccc54)c([2H])c([2H])c([2H])c32)c1[2H] ; [2H]c1cc(-c2nc(-c3ccccc3)nc(-c3ccccc3)n2)c([2H])c(-n2c3c([2H])cc([2H])c([2H])c3c3c([2H])c([2H])c([2H])c([2H])c32)c1-c1c([2H])c([2H])c([2H])c2c1oc1c([2H])c([2H])c([2H])c([2H])c12 ; [2H]c1cc([2H])c2oc3c(-c4ccc(-c5nc(-c6ccccc6)nc(-c6ccc(-c7ccccc7)cc6)n5)cc4-n4c5ccccc5c5ccccc54)c([2H])c([2H])c([2H])c3c2c1[2H]]

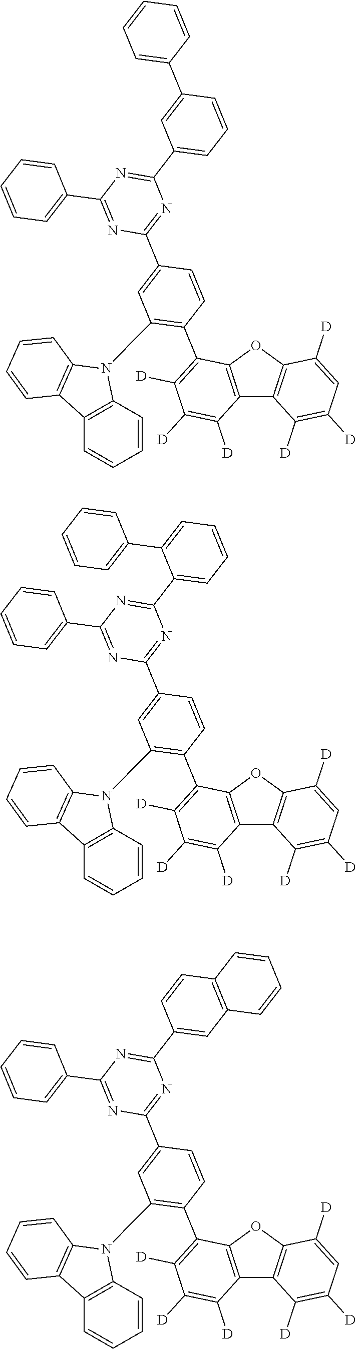

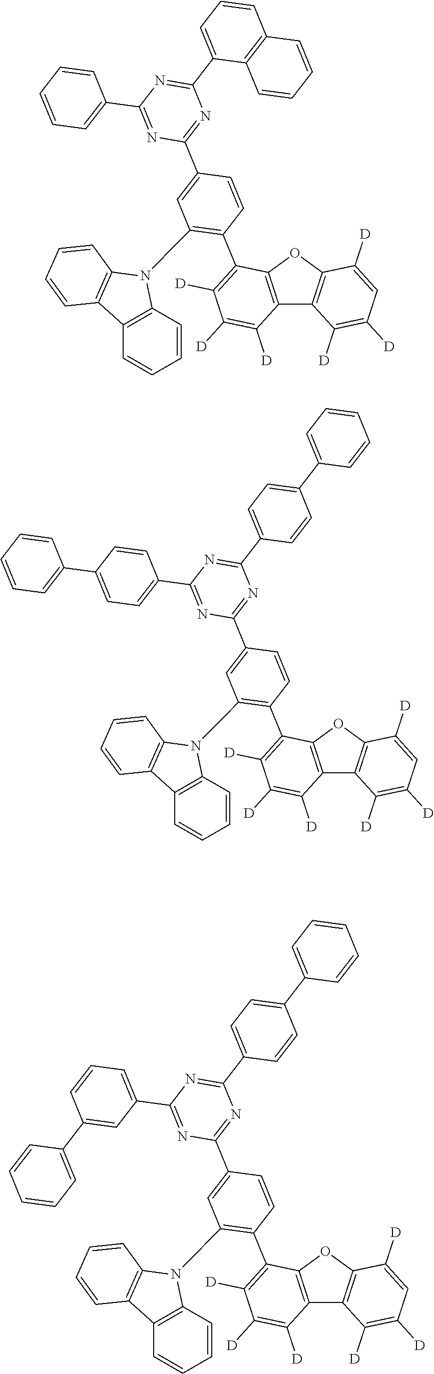 [SMILES: [2H]c1cc([2H])c2oc3c(-c4ccc(-c5nc(-c6ccccc6)nc(-c6cccc7ccccc67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccc(-c7ccccc7)cc6)nc(-c6ccc(-c7ccccc7)cc6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccc(-c7ccccc7)cc6)nc(-c6cccc(-c7ccccc7)c6)n5)cc4-n4c5ccccc5c5ccccc54)c([2H])c([2H])c([2H])c3c2c1[2H]]

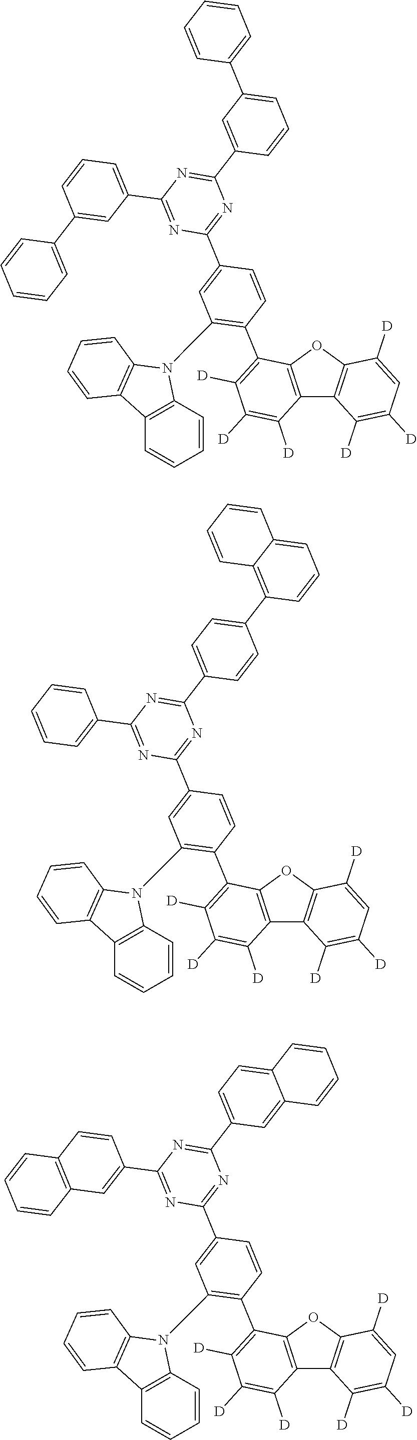 [SMILES: [2H]c1cc([2H])c2oc3c(-c4ccc(-c5nc(-c6cccc(-c7ccccc7)c6)nc(-c6cccc(-c7ccccc7)c6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccc(-c7cccc8ccccc78)cc6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccc7ccccc7c6)nc(-c6ccc7ccccc7c6)n5)cc4-n4c5ccccc5c5ccccc54)c([2H])c([2H])c([2H])c3c2c1[2H]]

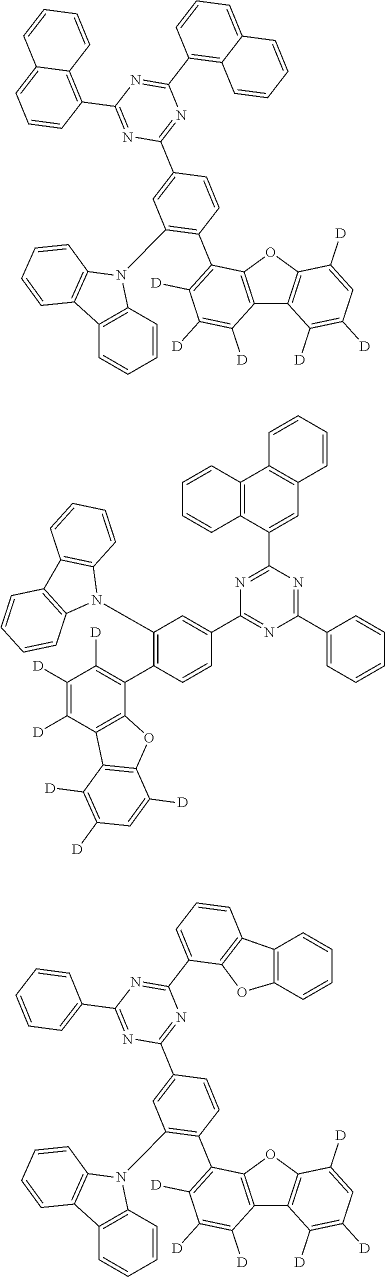 [SMILES: [2H]c1cc([2H])c2oc3c(-c4ccc(-c5nc(-c6cccc7ccccc67)nc(-c6cccc7ccccc67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6cc7ccccc7c7ccccc67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6cccc7c6oc6ccccc67)n5)cc4-n4c5ccccc5c5ccccc54)c([2H])c([2H])c([2H])c3c2c1[2H]]

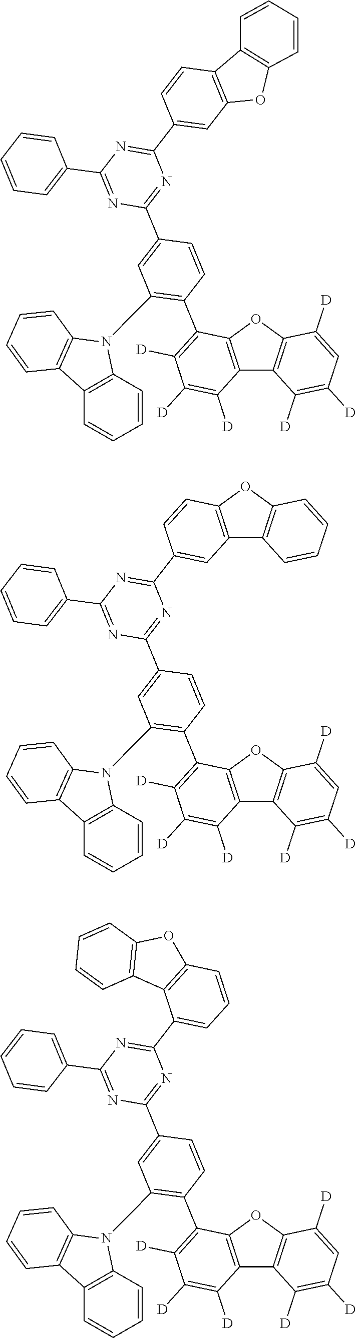 [SMILES: [2H]c1cc([2H])c2oc3c(-c4ccc(-c5nc(-c6ccccc6)nc(-c6ccc7c(c6)oc6ccccc67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccc7oc8ccccc8c7c6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6cccc7oc8ccccc8c67)n5)cc4-n4c5ccccc5c5ccccc54)c([2H])c([2H])c([2H])c3c2c1[2H]]

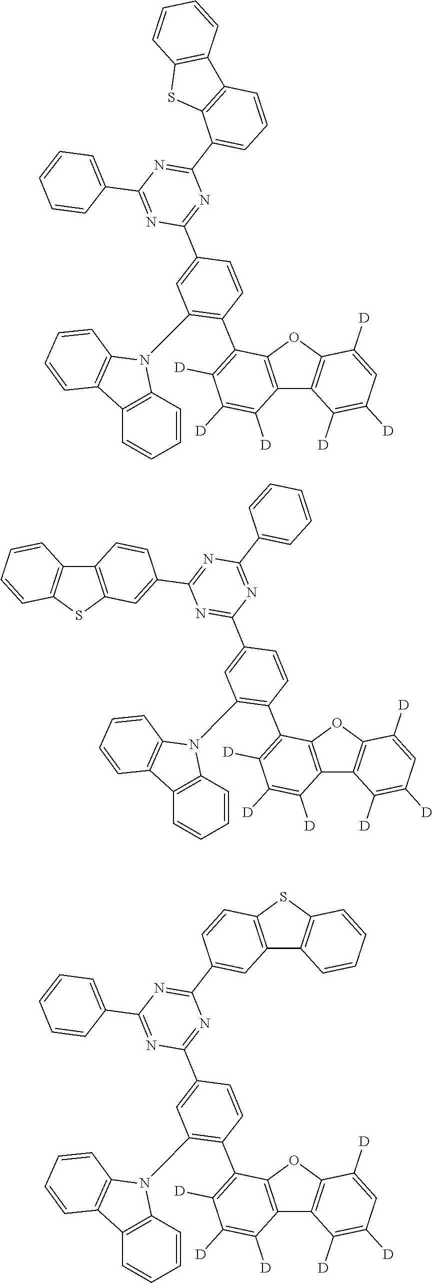

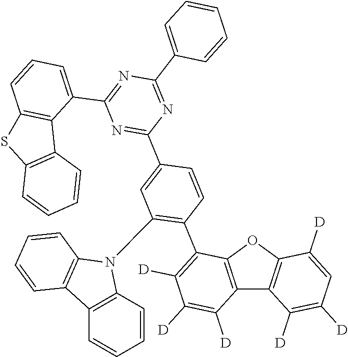 [SMILES: [2H]c1cc([2H])c2oc3c(-c4ccc(-c5nc(-c6ccccc6)nc(-c6cccc7sc8ccccc8c67)n5)cc4-n4c5ccccc5c5ccccc54)c([2H])c([2H])c([2H])c3c2c1[2H]]

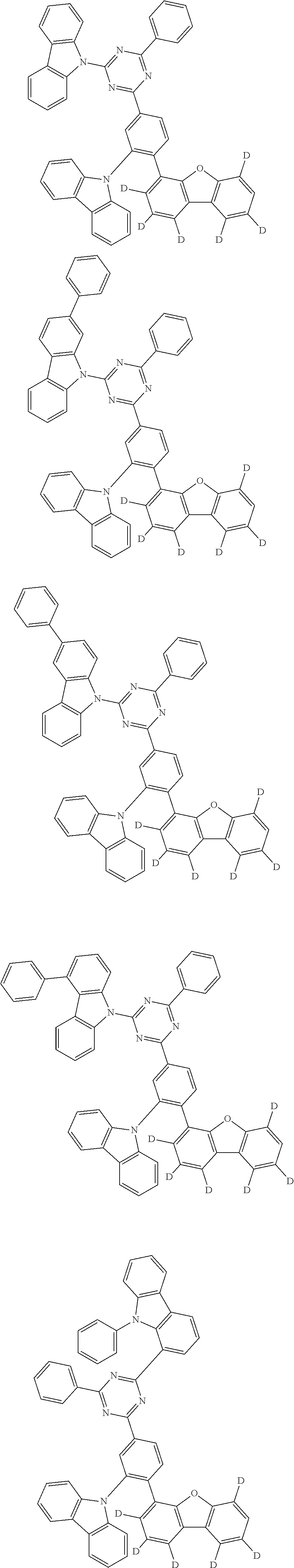

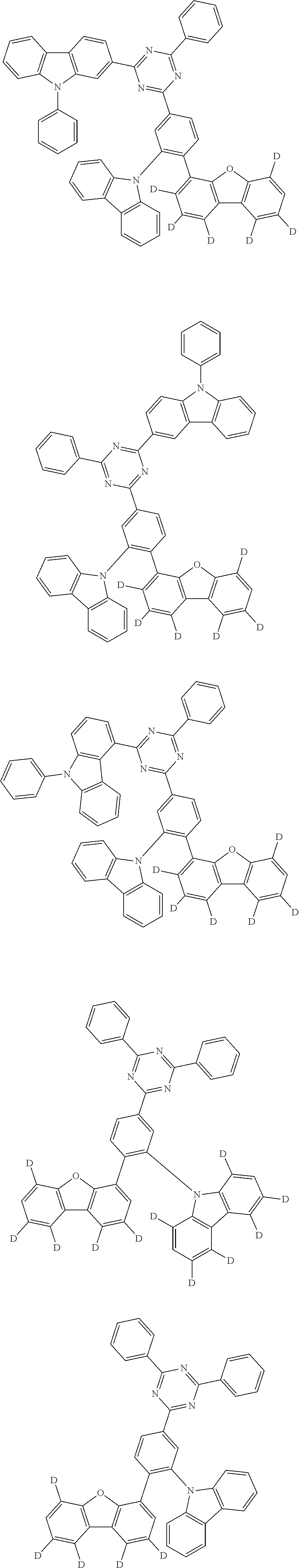

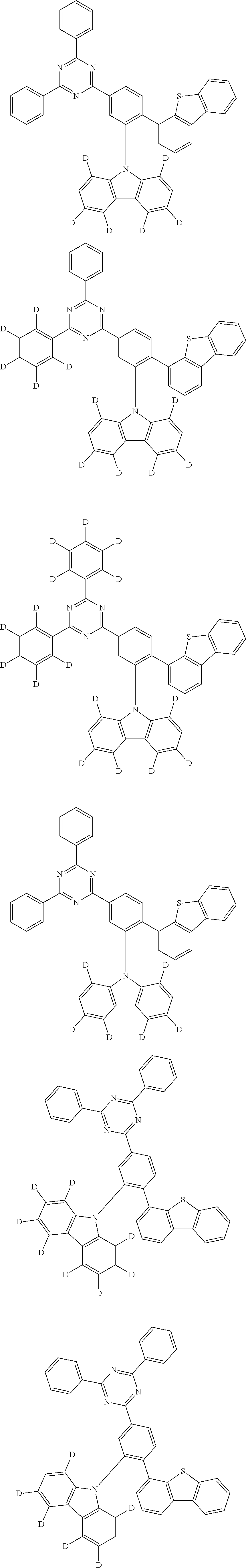

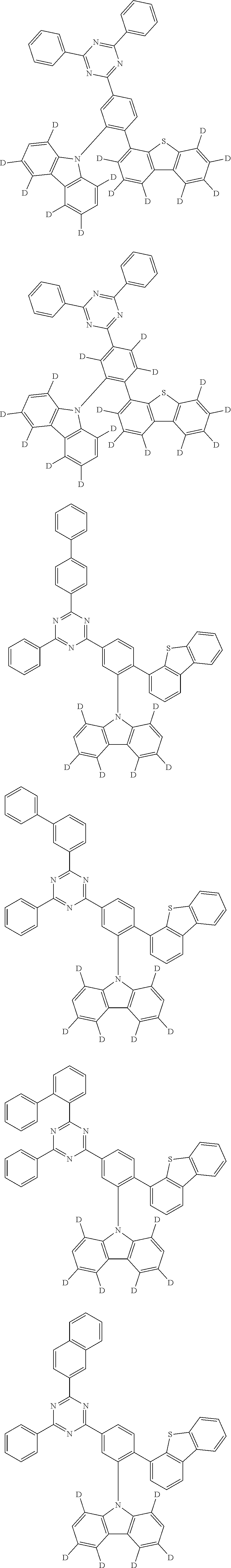

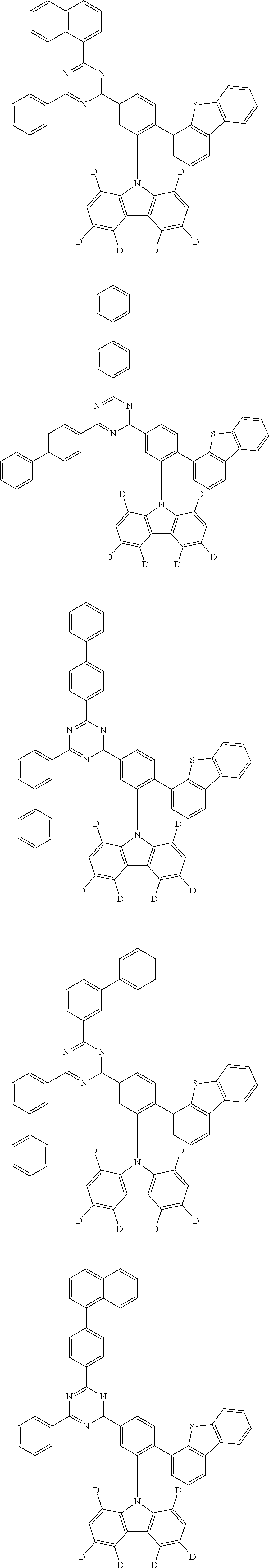

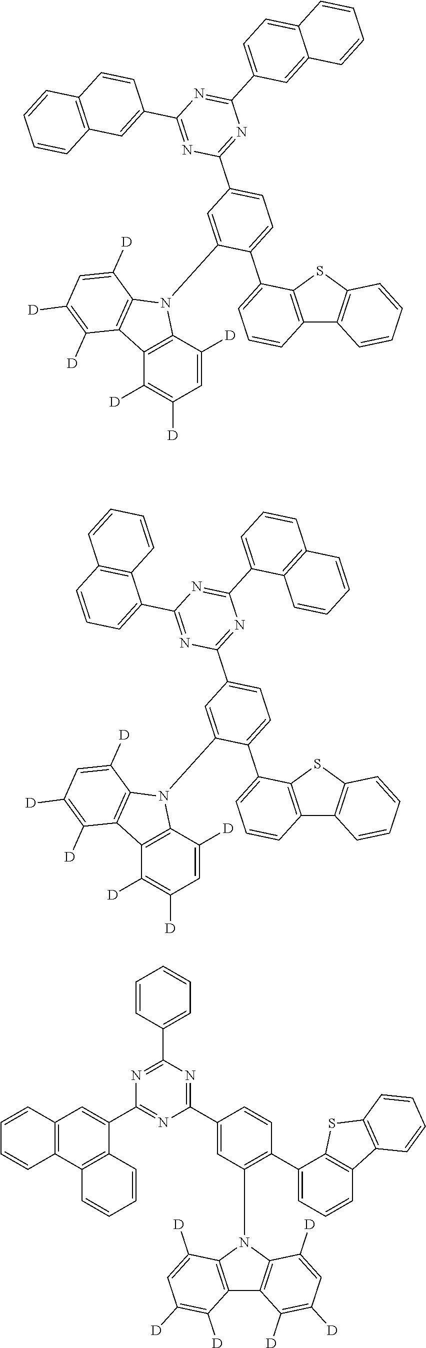

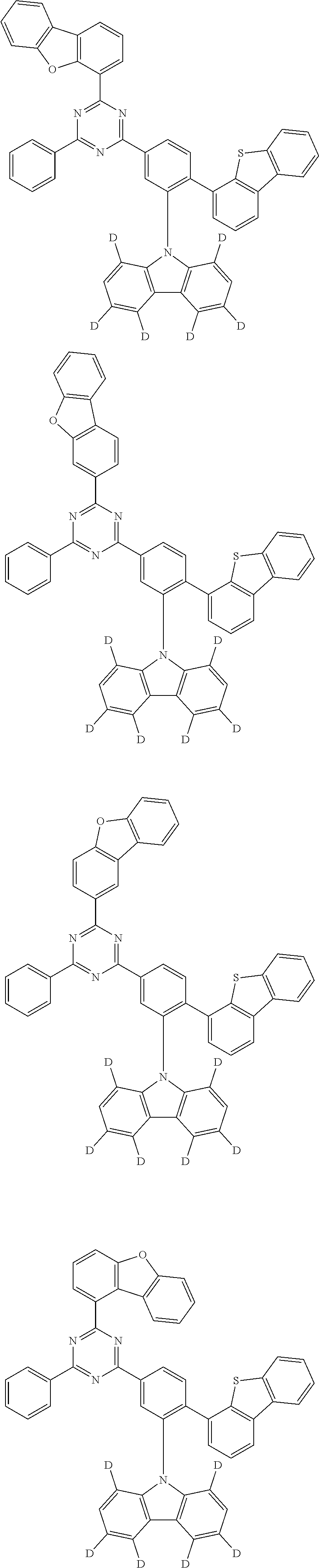

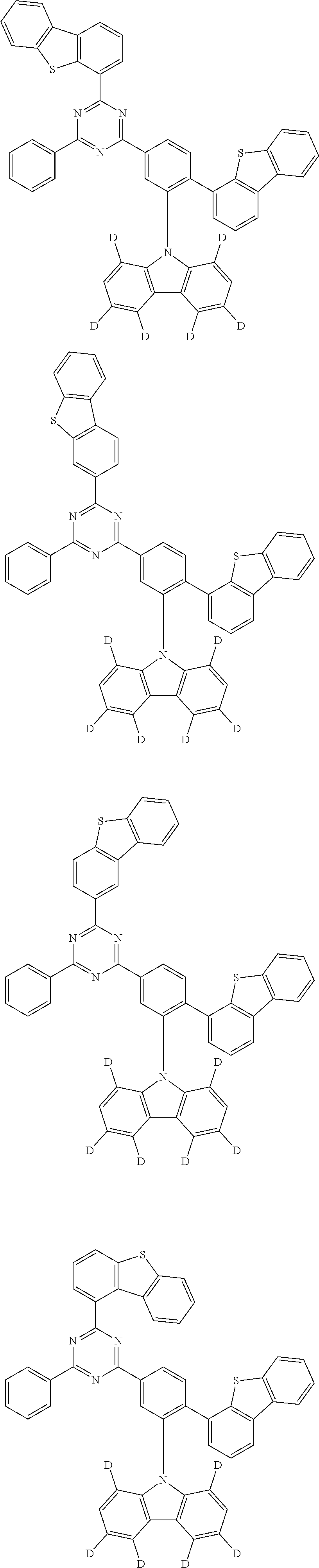

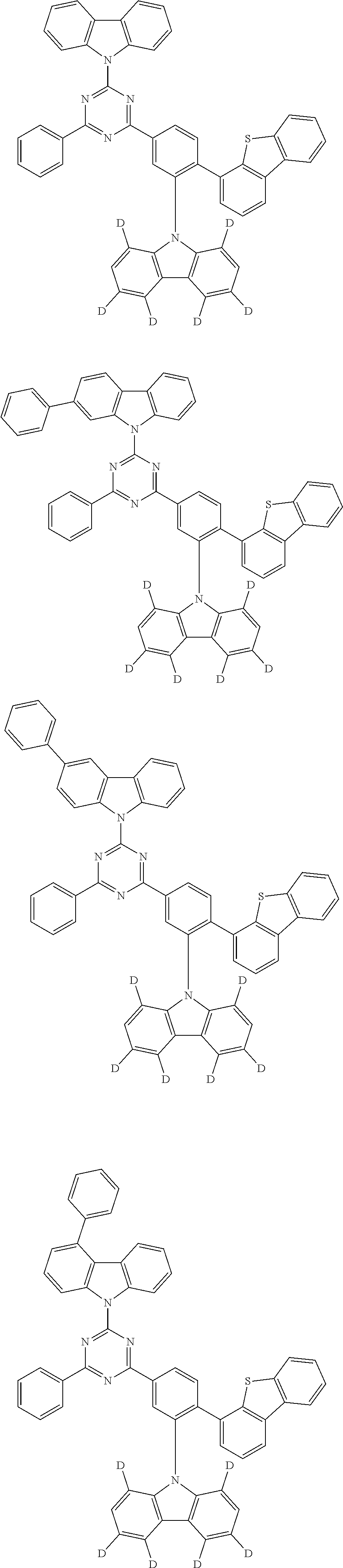

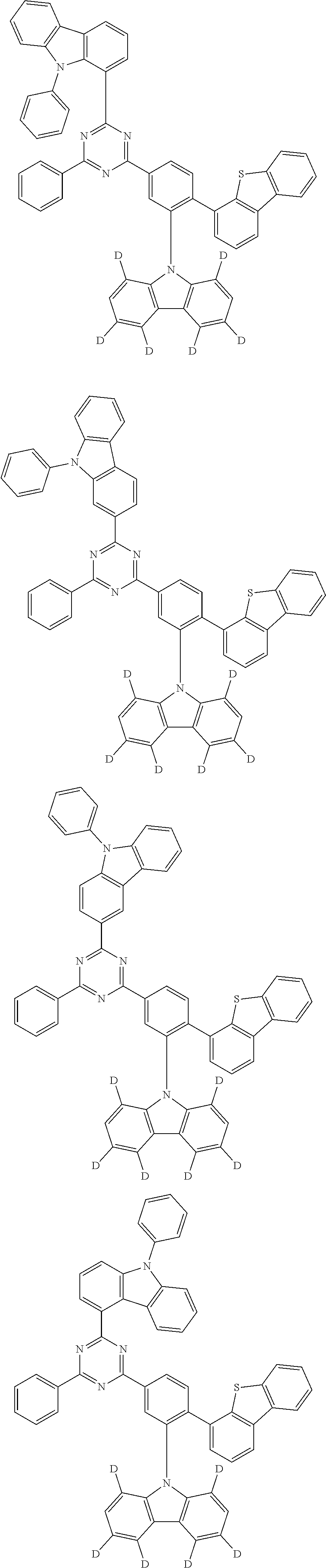

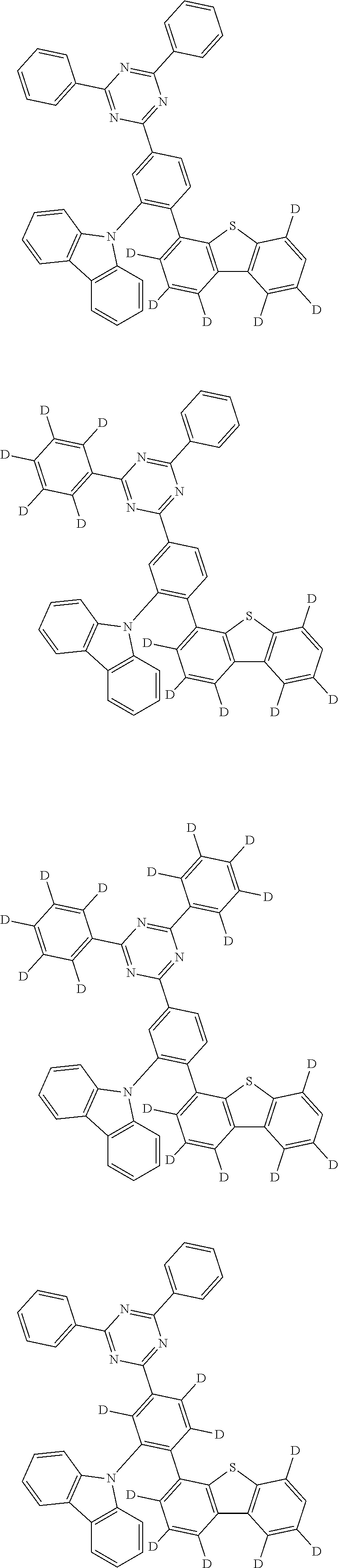

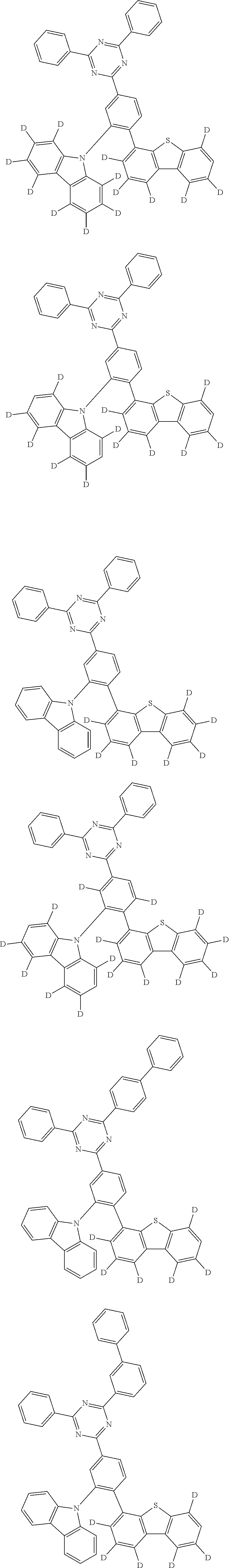

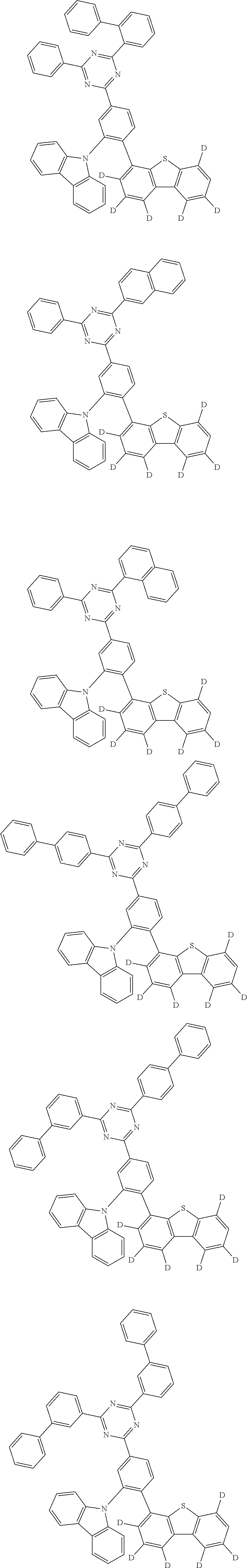

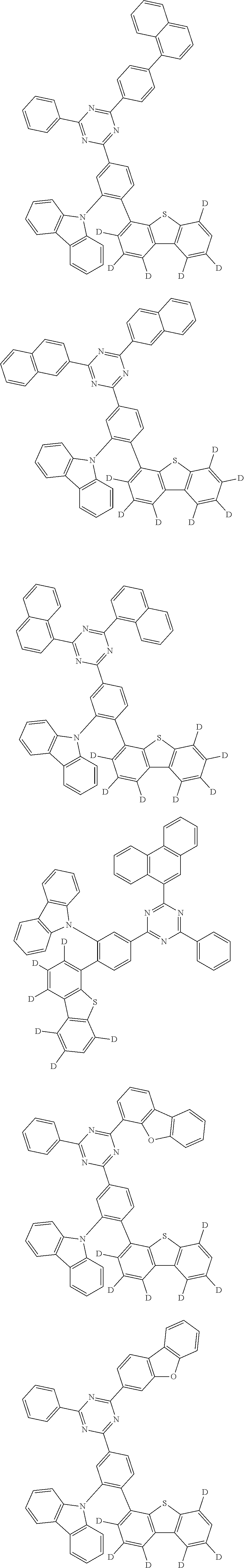

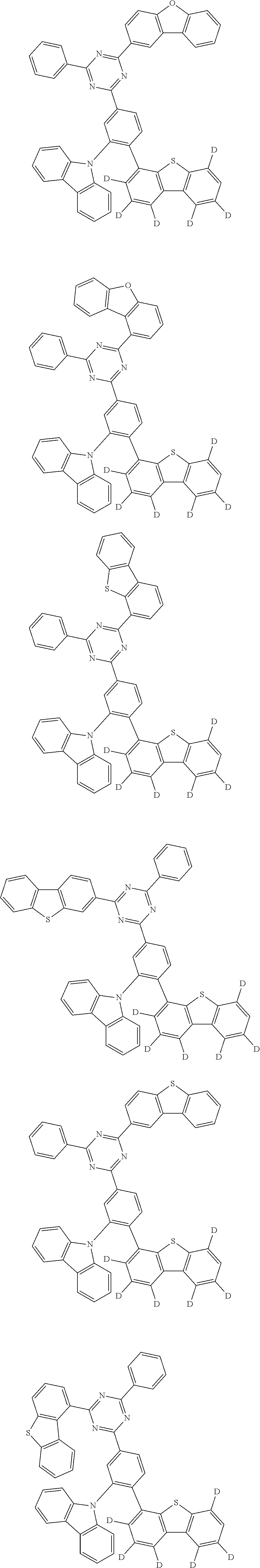

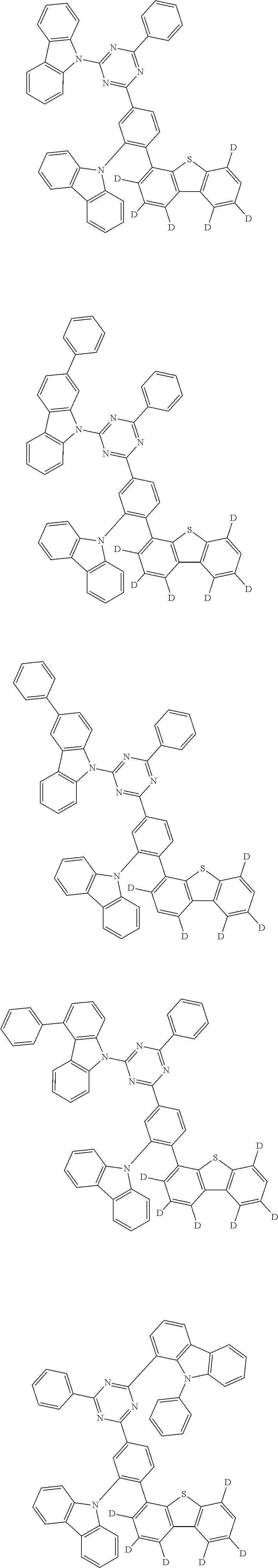

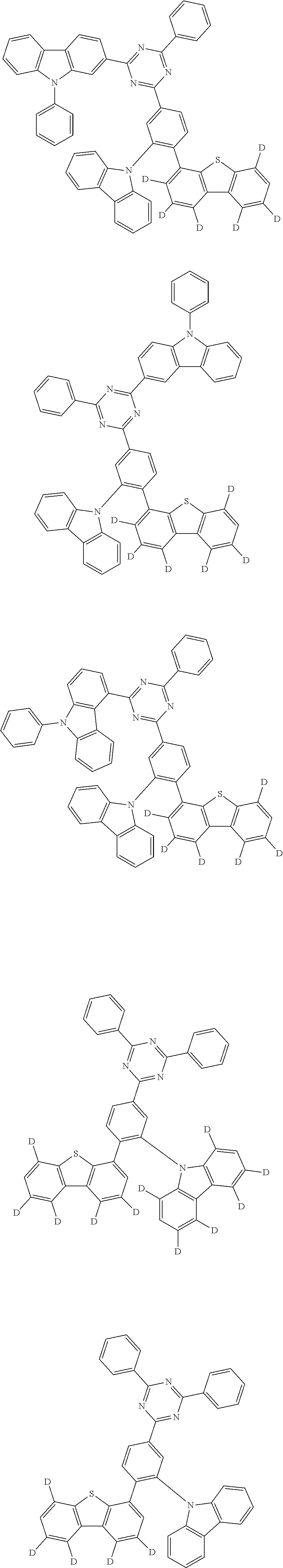

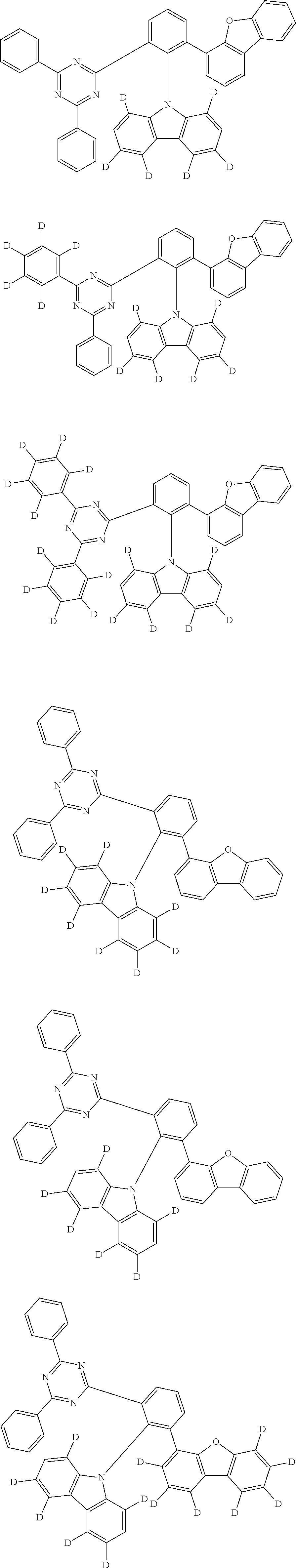

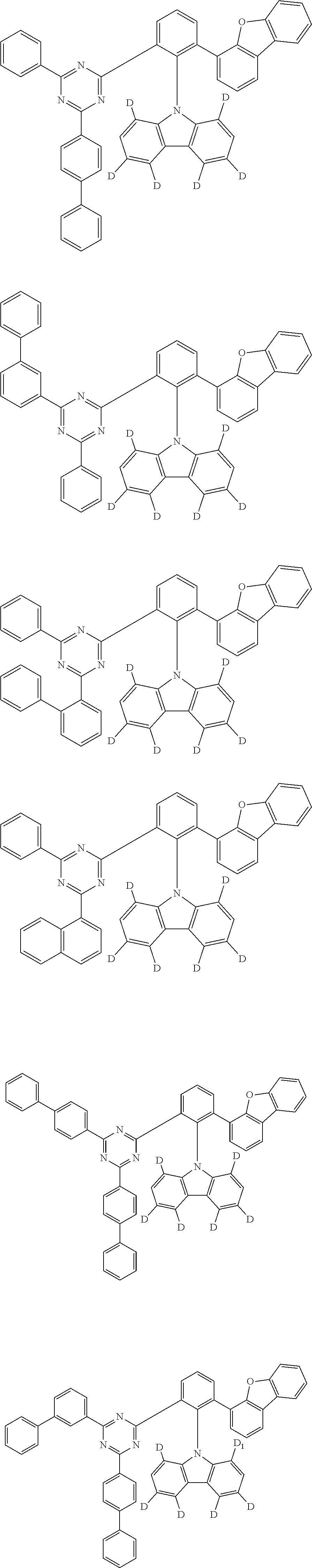

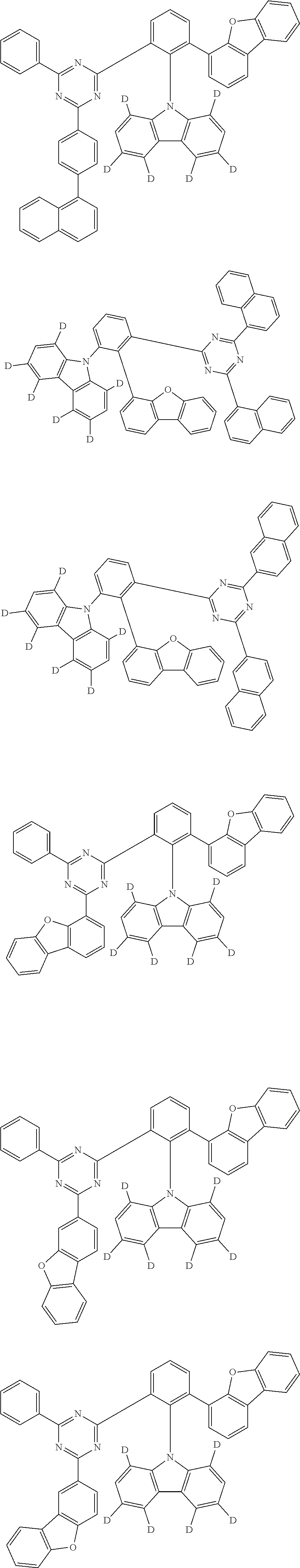

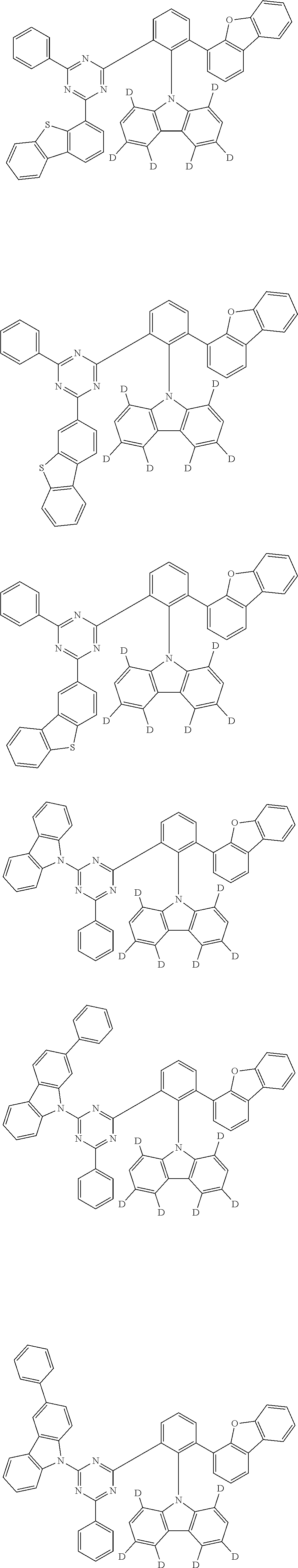

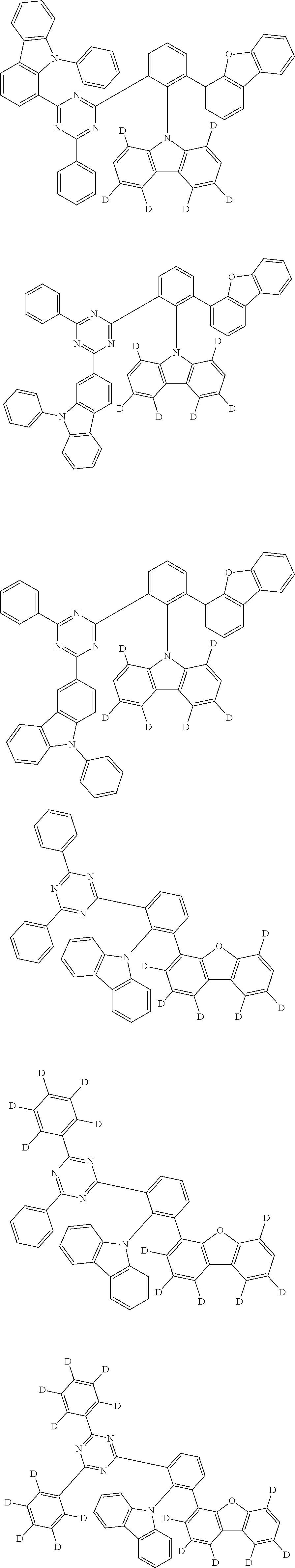

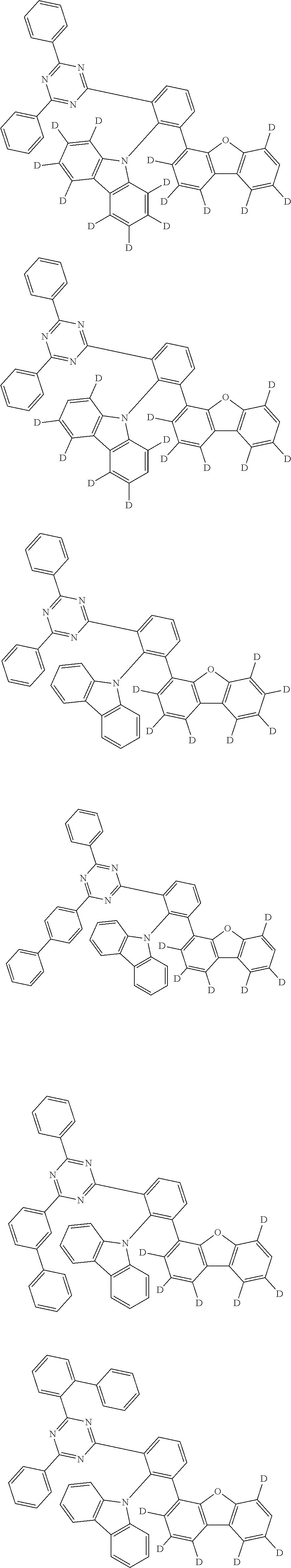

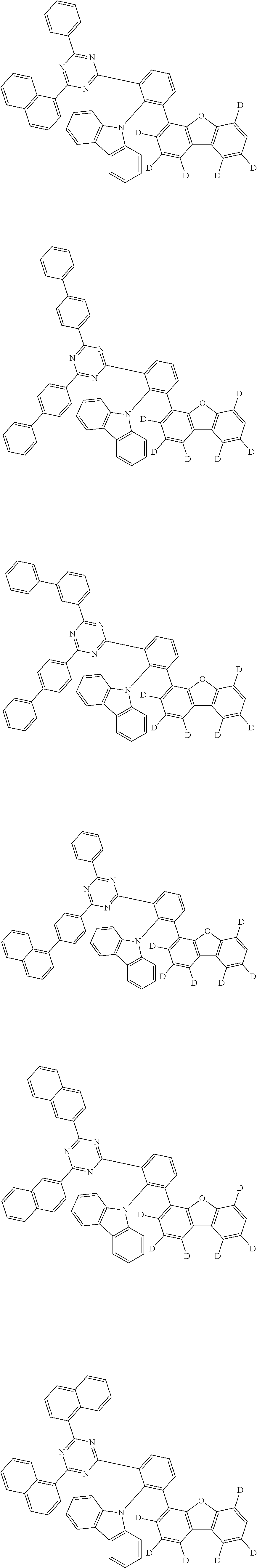

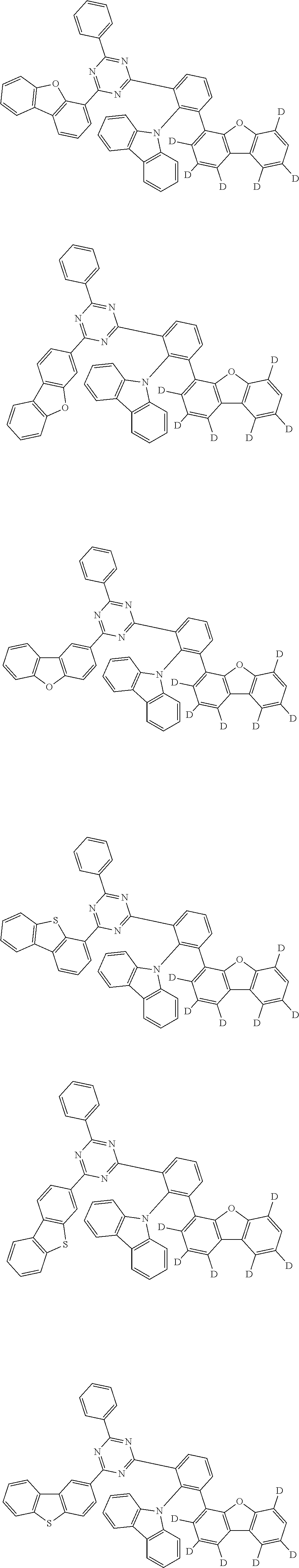

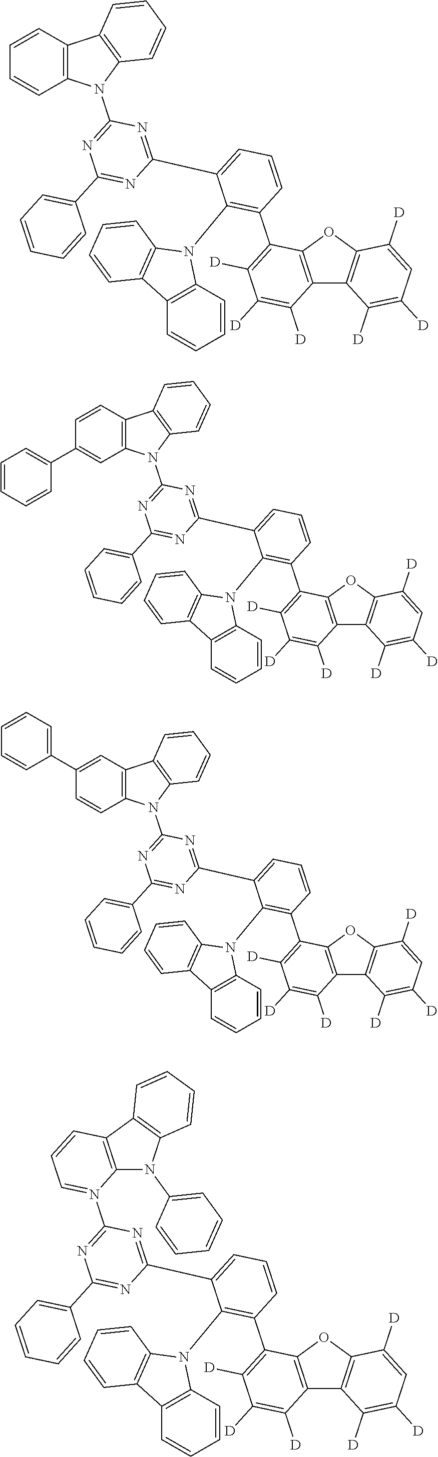

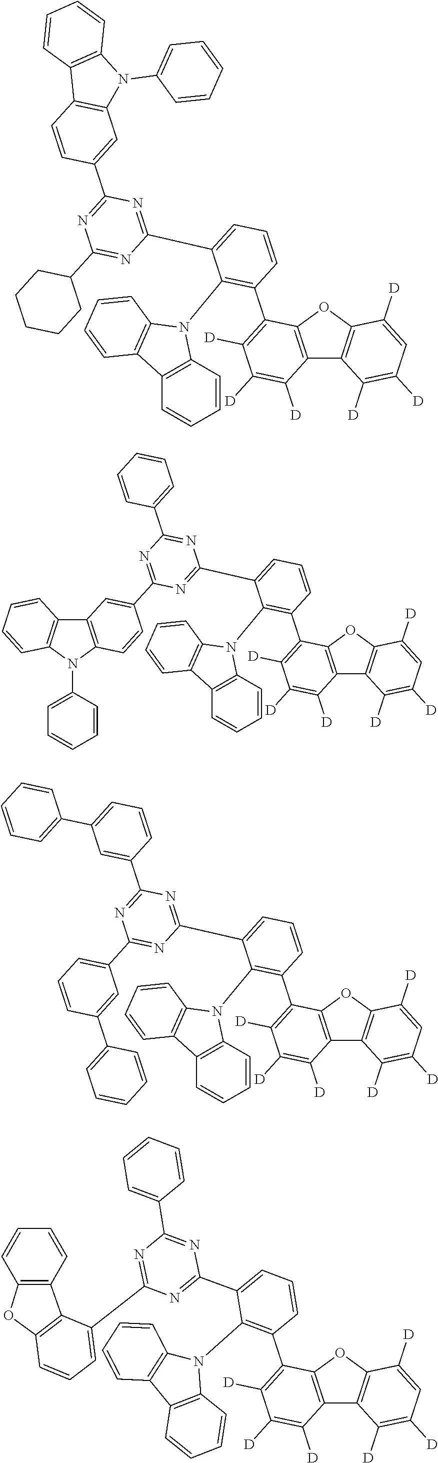

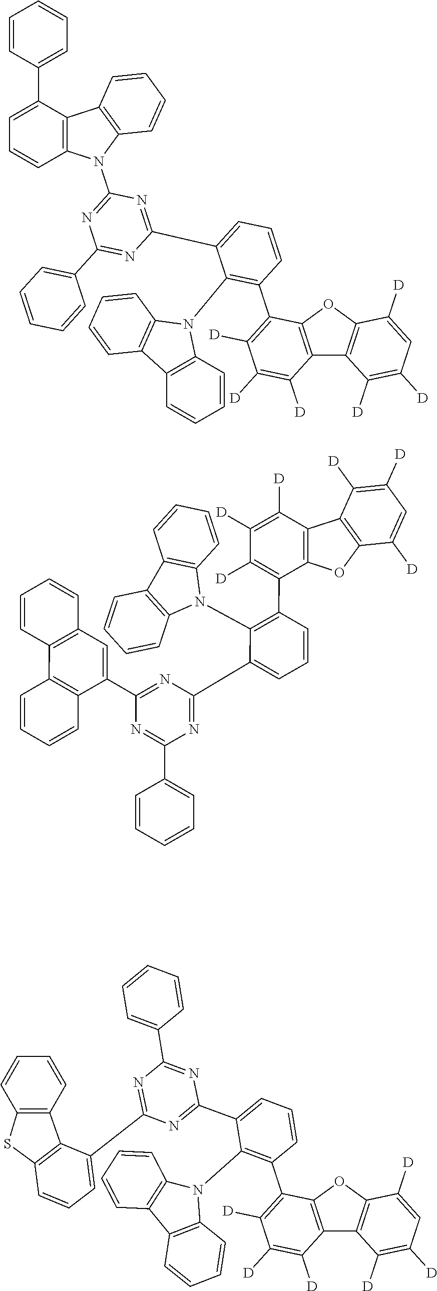 [SMILES: [2H]c1cc([2H])c2oc3c(-c4cccc(-c5nc(-c6ccccc6)nc(-n6c7ccccc7c7c(-c8ccccc8)cccc76)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6cc7ccccc7c7ccccc67)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6cccc7sc8ccccc8c67)n5)c4-n4c5ccccc5c5ccccc54)c([2H])c([2H])c([2H])c3c2c1[2H]]

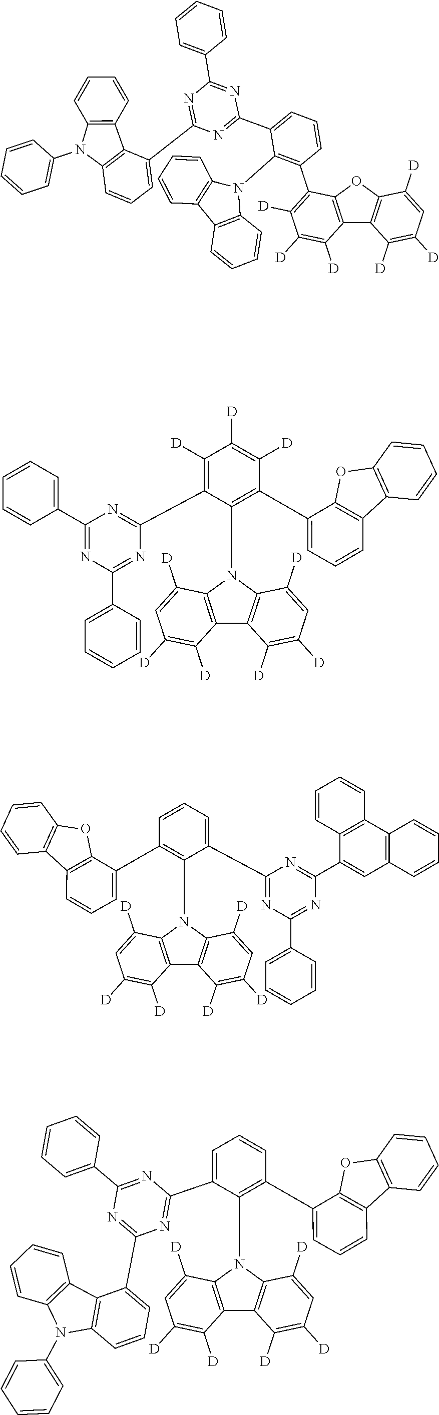

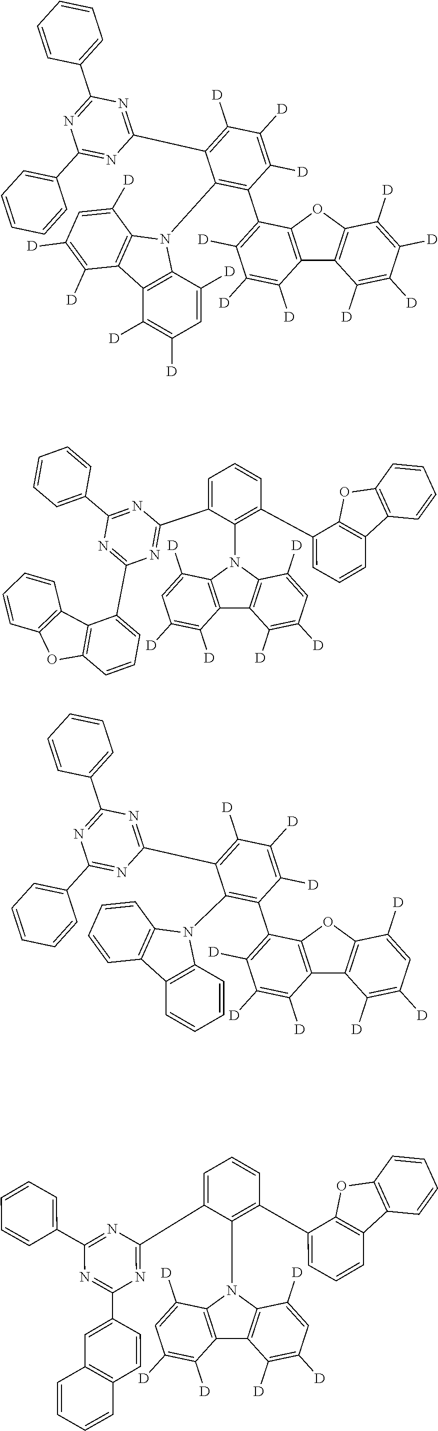

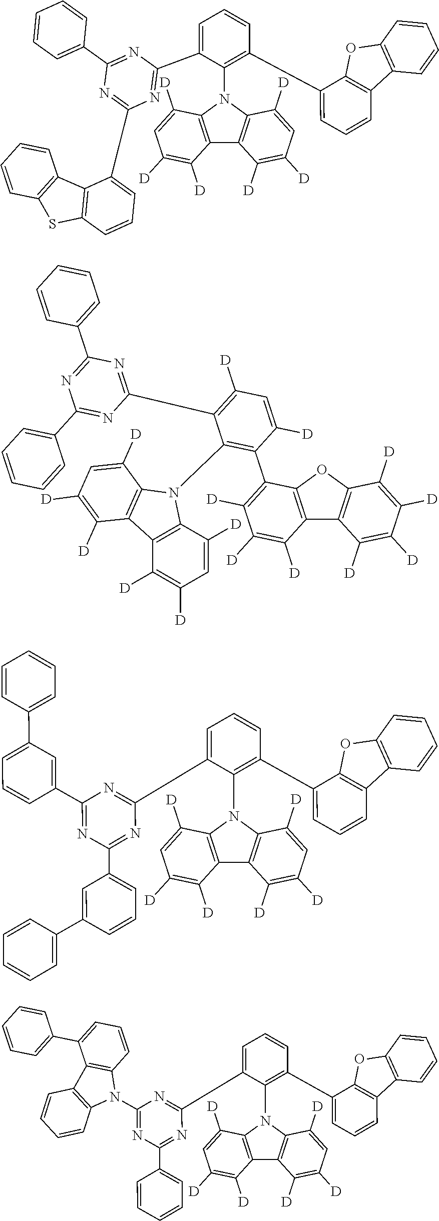

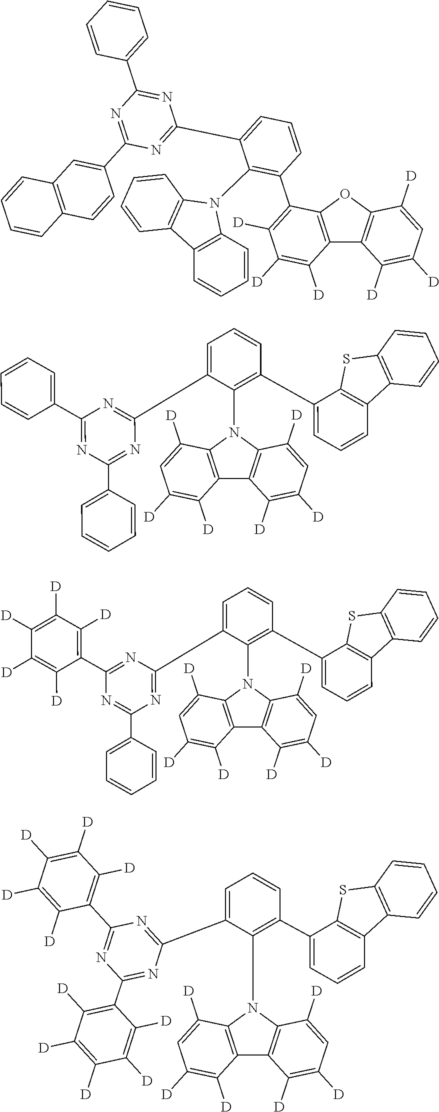

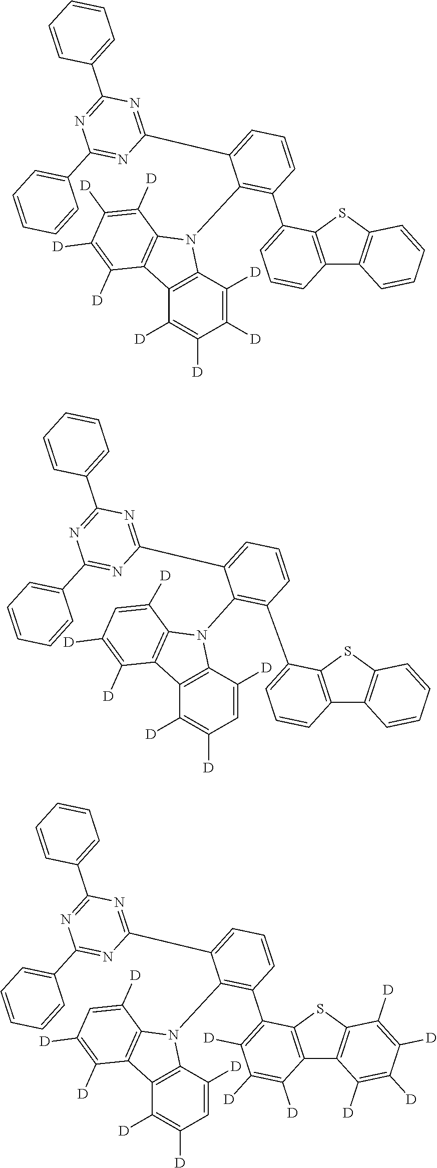 [SMILES: [2H]c1c([2H])c([2H])c2c(c1[2H])c1c([2H])c([2H])c([2H])c([2H])c1n2-c1c(-c2nc(-c3ccccc3)nc(-c3ccccc3)n2)cccc1-c1cccc2c1sc1ccccc12 ; [2H]c1cc([2H])c2c(c1[2H])c1c([2H])c([2H])cc([2H])c1n2-c1c(-c2nc(-c3ccccc3)nc(-c3ccccc3)n2)cccc1-c1cccc2c1sc1ccccc12 ; [2H]c1cc([2H])c2c(c1[2H])c1c([2H])c([2H])cc([2H])c1n2-c1c(-c2nc(-c3ccccc3)nc(-c3ccccc3)n2)cccc1-c1c([2H])c([2H])c([2H])c2c1sc1c([2H])c([2H])c([2H])c([2H])c12]

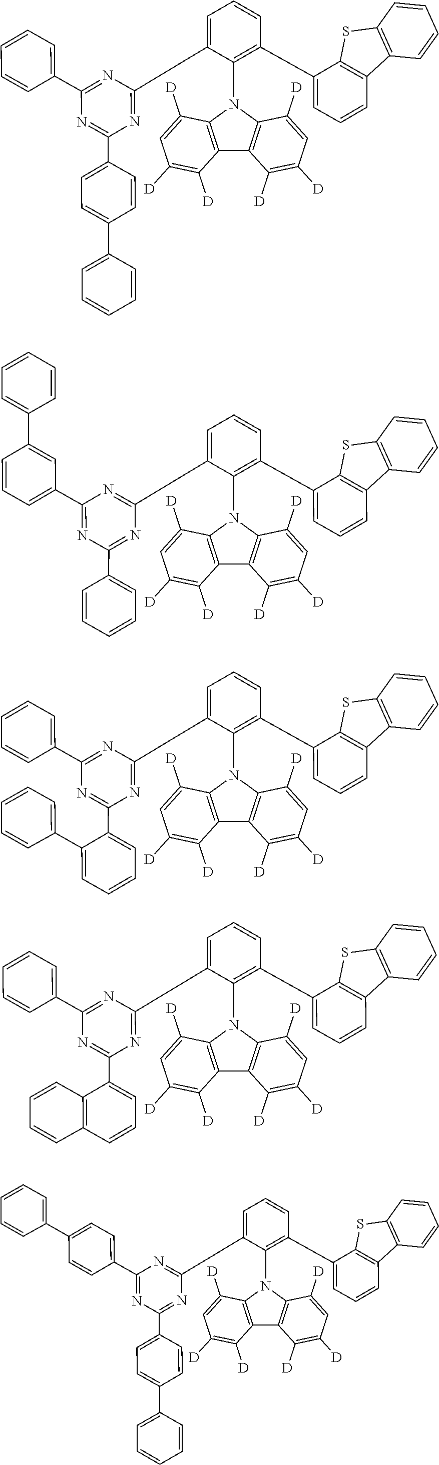

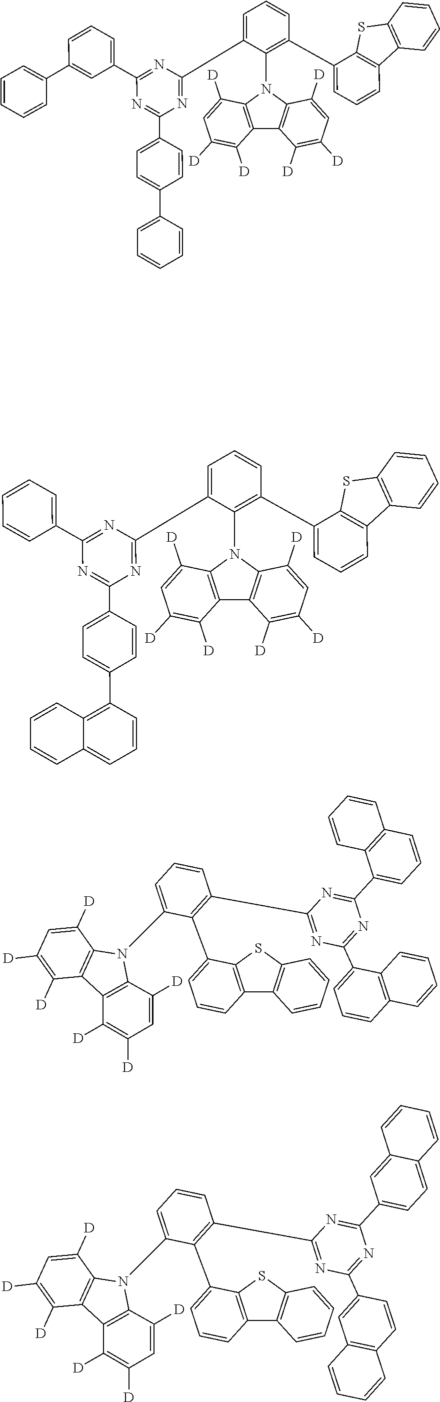

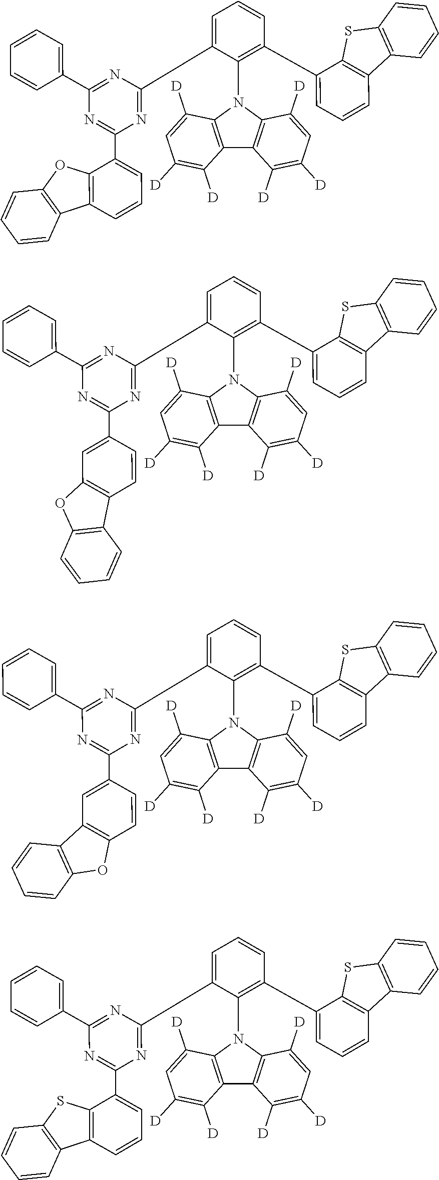

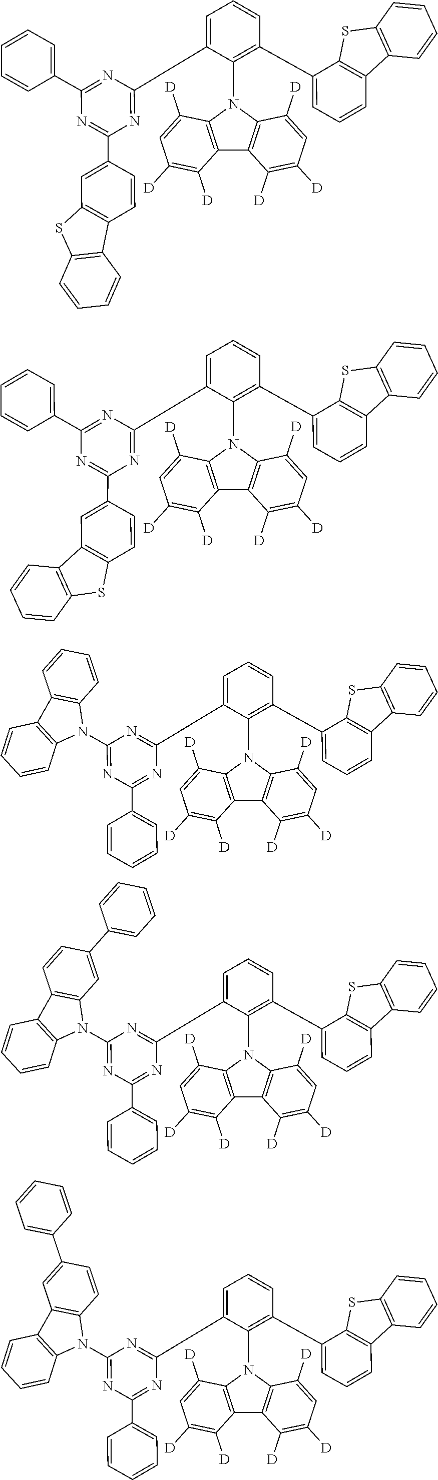

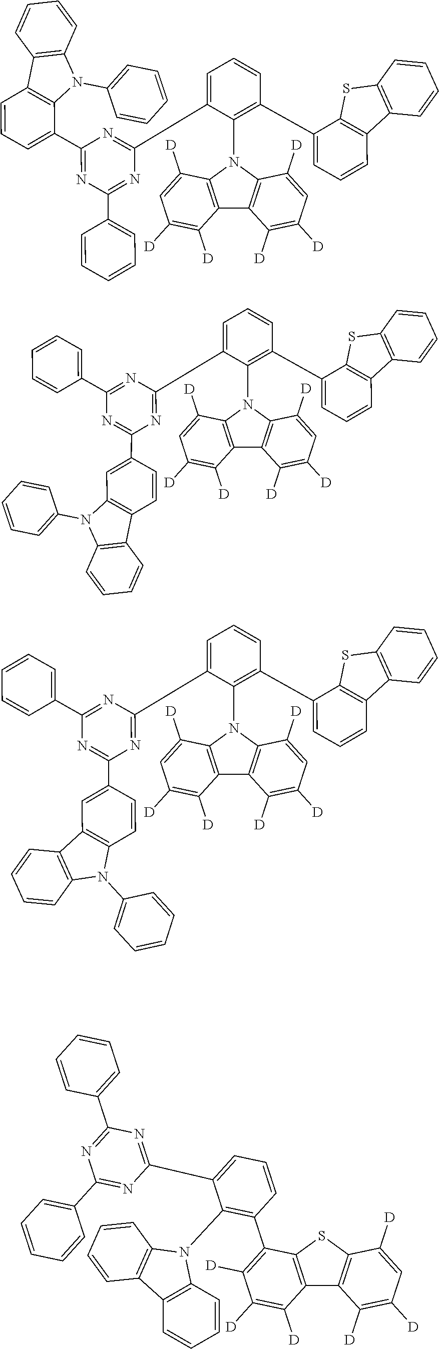

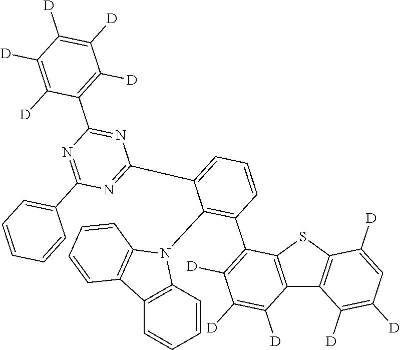 [SMILES: [2H]c1cc([2H])c2sc3c(-c4cccc(-c5nc(-c6ccccc6)nc(-c6c([2H])c([2H])c([2H])c([2H])c6[2H])n5)c4-n4c5ccccc5c5ccccc54)c([2H])c([2H])c([2H])c3c2c1[2H]]

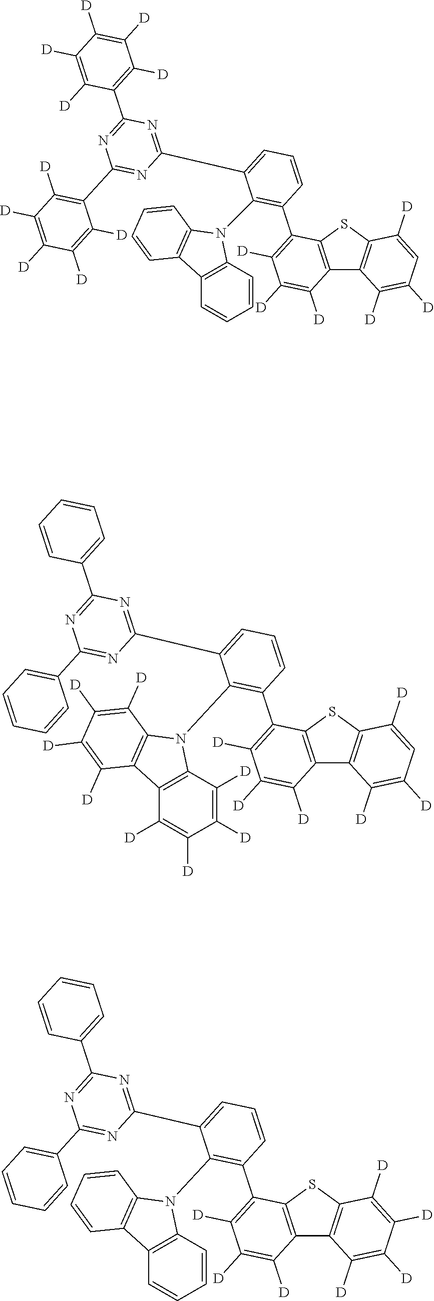 [SMILES: [2H]c1cc([2H])c2sc3c(-c4cccc(-c5nc(-c6c([2H])c([2H])c([2H])c([2H])c6[2H])nc(-c6c([2H])c([2H])c([2H])c([2H])c6[2H])n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccccc6)n5)c4-n4c5c([2H])c([2H])c([2H])c([2H])c5c5c([2H])c([2H])c([2H])c([2H])c54)c([2H])c([2H])c([2H])c3c2c1[2H] ; [2H]c1c([2H])c([2H])c2c(sc3c(-c4cccc(-c5nc(-c6ccccc6)nc(-c6ccccc6)n5)c4-n4c5ccccc5c5ccccc54)c([2H])c([2H])c([2H])c32)c1[2H]]

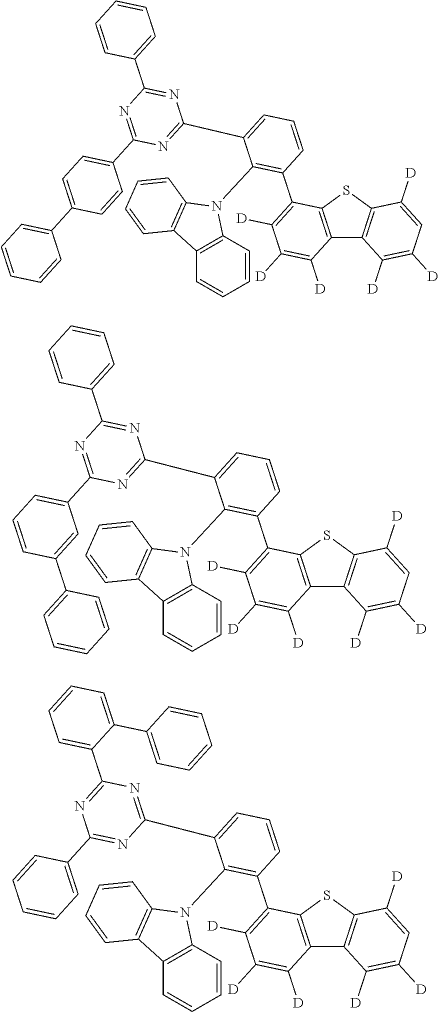

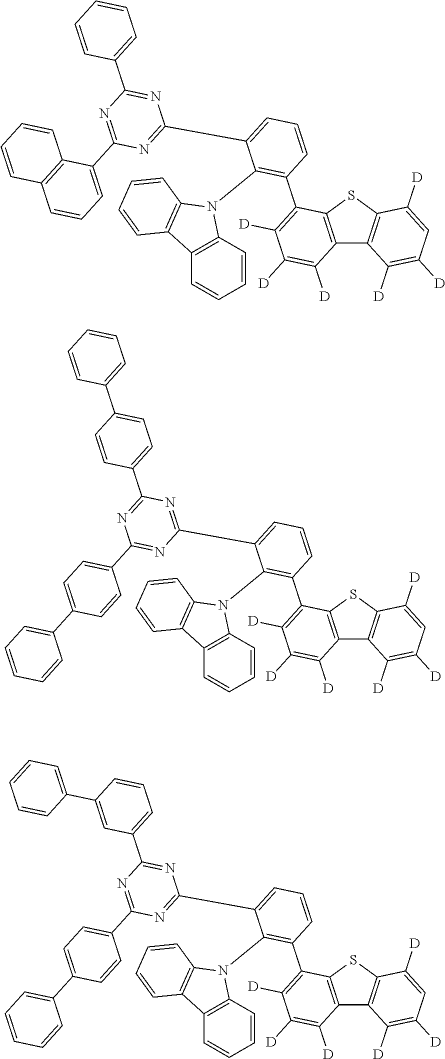 [SMILES: [2H]c1cc([2H])c2sc3c(-c4cccc(-c5nc(-c6ccccc6)nc(-c6cccc7ccccc67)n5)c4-n4c5ccccc5c5ccccc54)c([2H])c([2H])c([2H])c3c2c1[2H] ; [2H]c1cc([2H])c2sc3c(-c4cccc(-c5nc(-c6ccc(-c7ccccc7)cc6)nc(-c6ccc(-c7ccccc7)cc6)n5)c4-n4c5ccccc5c5ccccc54)c([2H])c([2H])c([2H])c3c2c1[2H] ; [2H]c1cc([2H])c2sc3c(-c4cccc(-c5nc(-c6ccc(-c7ccccc7)cc6)nc(-c6cccc(-c7ccccc7)c6)n5)c4-n4c5ccccc5c5ccccc54)c([2H])c([2H])c([2H])c3c2c1[2H]]

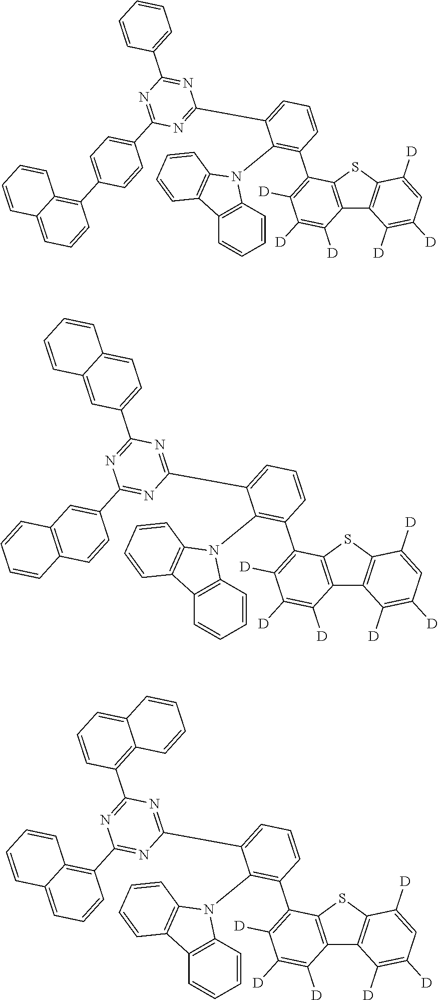 [SMILES: [2H]c1cc([2H])c2sc3c(-c4cccc(-c5nc(-c6ccccc6)nc(-c6ccc(-c7cccc8ccccc78)cc6)n5)c4-n4c5ccccc5c5ccccc54)c([2H])c([2H])c([2H])c3c2c1[2H] ; [2H]c1cc([2H])c2sc3c(-c4cccc(-c5nc(-c6ccc7ccccc7c6)nc(-c6ccc7ccccc7c6)n5)c4-n4c5ccccc5c5ccccc54)c([2H])c([2H])c([2H])c3c2c1[2H] ; [2H]c1cc([2H])c2sc3c(-c4cccc(-c5nc(-c6cccc7ccccc67)nc(-c6cccc7ccccc67)n5)c4-n4c5ccccc5c5ccccc54)c([2H])c([2H])c([2H])c3c2c1[2H]]

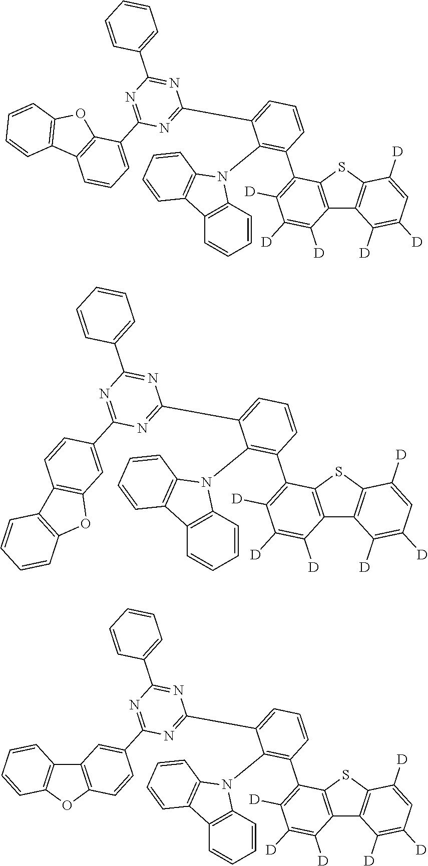 [SMILES: [2H]c1cc([2H])c2sc3c(-c4cccc(-c5nc(-c6ccccc6)nc(-c6cccc7c6oc6ccccc67)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccc7c(c6)oc6ccccc67)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccc7oc8ccccc8c7c6)n5)c4-n4c5ccccc5c5ccccc54)c([2H])c([2H])c([2H])c3c2c1[2H]]

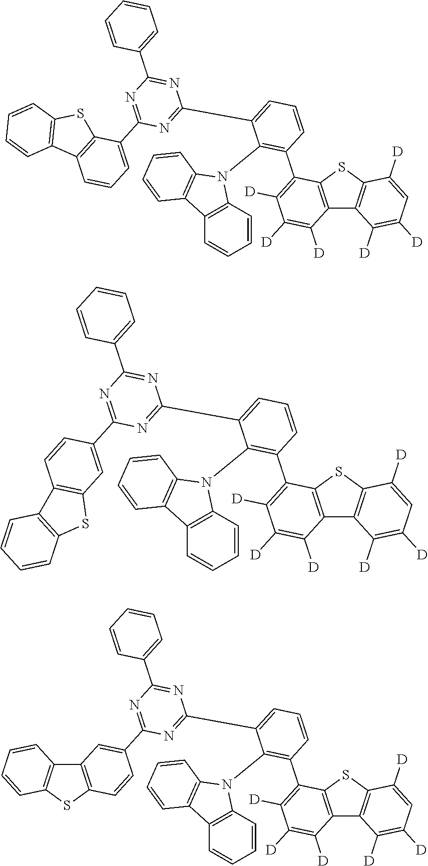 [SMILES: [2H]c1cc([2H])c2sc3c(-c4cccc(-c5nc(-c6ccccc6)nc(-c6cccc7c6sc6ccccc67)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccc7c(c6)sc6ccccc67)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccc7sc8ccccc8c7c6)n5)c4-n4c5ccccc5c5ccccc54)c([2H])c([2H])c([2H])c3c2c1[2H]]

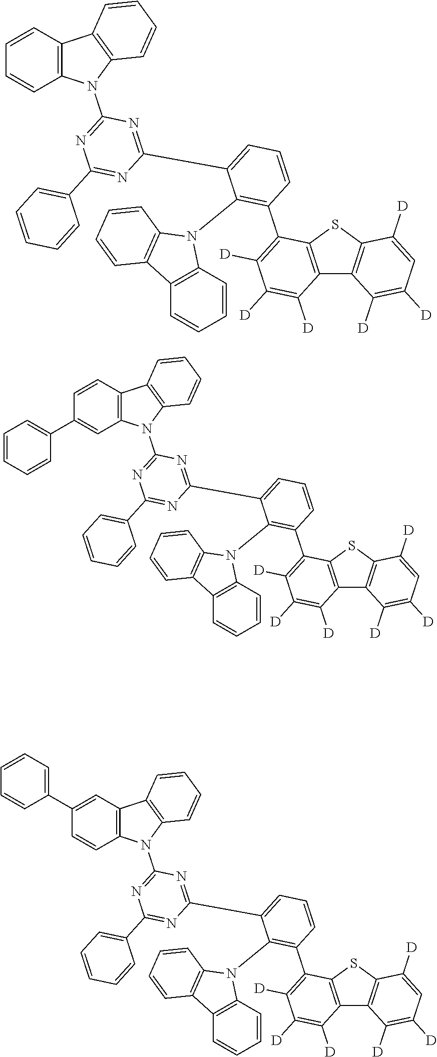

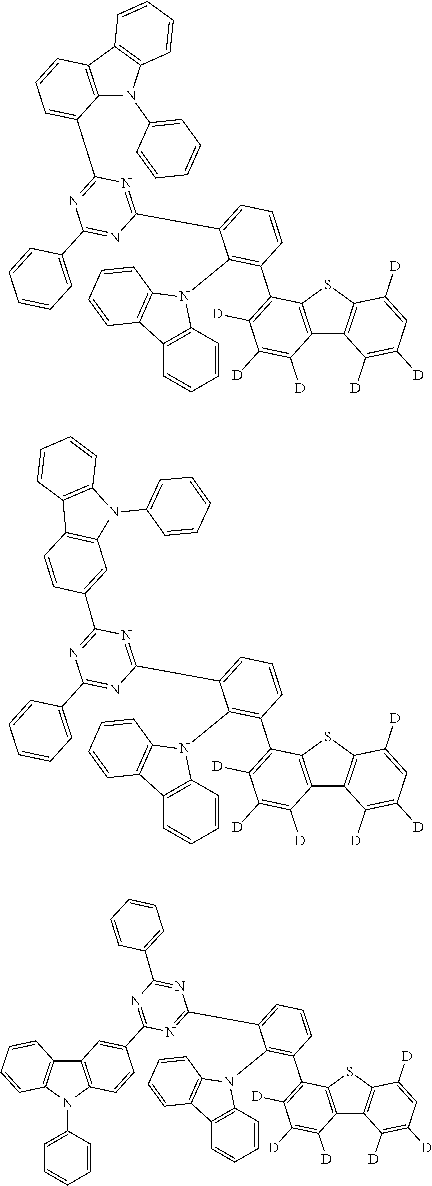 [SMILES: [2H]c1cc([2H])c2sc3c(-c4cccc(-c5nc(-c6ccccc6)nc(-c6cccc7c8ccccc8n(-c8ccccc8)c67)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccc7c8ccccc8n(-c8ccccc8)c7c6)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccc7c(c6)c6ccccc6n7-c6ccccc6)n5)c4-n4c5ccccc5c5ccccc54)c([2H])c([2H])c([2H])c3c2c1[2H]]

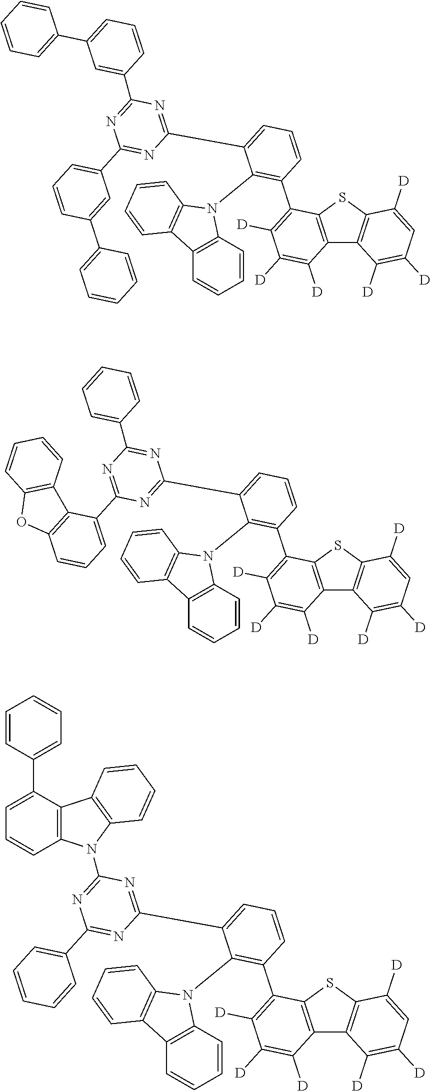 [SMILES: [2H]c1cc([2H])c2sc3c(-c4cccc(-c5nc(-c6cccc(-c7ccccc7)c6)nc(-c6cccc(-c7ccccc7)c6)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6cccc7oc8ccccc8c67)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-n6c7ccccc7c7c(-c8ccccc8)cccc76)n5)c4-n4c5ccccc5c5ccccc54)c([2H])c([2H])c([2H])c3c2c1[2H]]

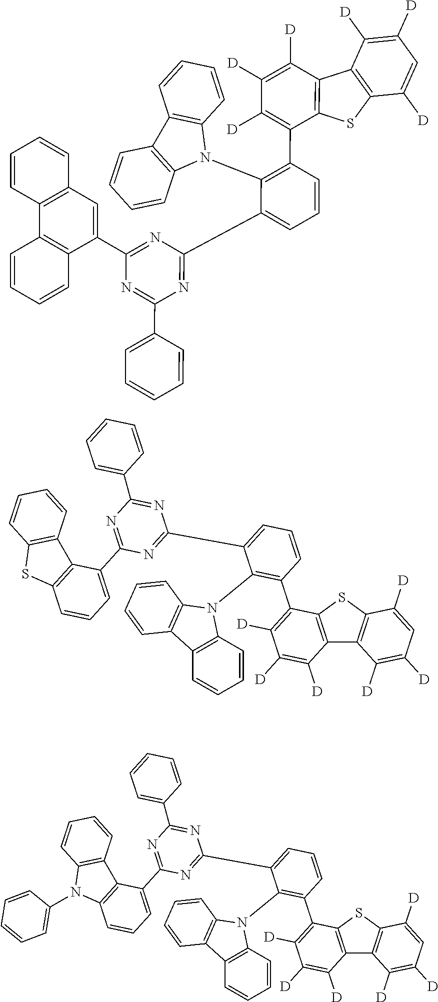

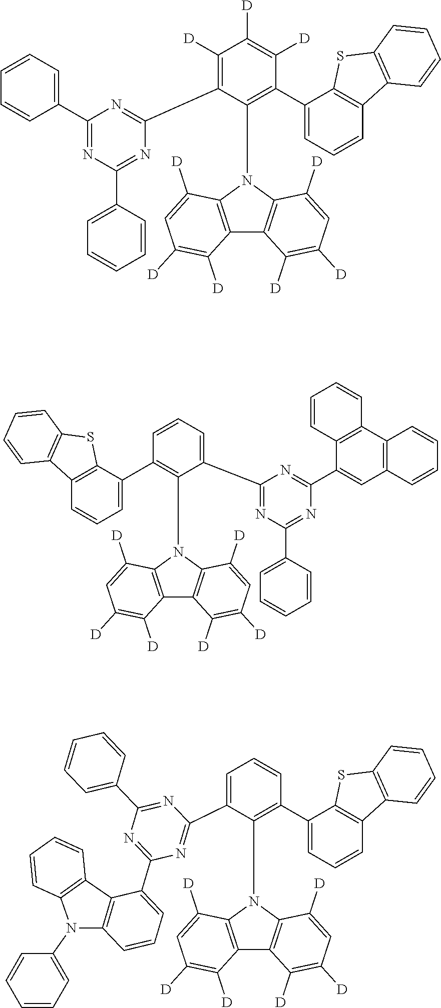 [SMILES: [2H]c1cc([2H])c2c(c1[2H])c1c([2H])c([2H])cc([2H])c1n2-c1c(-c2nc(-c3ccccc3)nc(-c3ccccc3)n2)c([2H])c([2H])c([2H])c1-c1cccc2c1sc1ccccc12 ; [2H]c1cc([2H])c2c(c1[2H])c1c([2H])c([2H])cc([2H])c1n2-c1c(-c2nc(-c3ccccc3)nc(-c3cc4ccccc4c4ccccc34)n2)cccc1-c1cccc2c1sc1ccccc12 ; [2H]c1cc([2H])c2c(c1[2H])c1c([2H])c([2H])cc([2H])c1n2-c1c(-c2nc(-c3ccccc3)nc(-c3cccc4c3c3ccccc3n4-c3ccccc3)n2)cccc1-c1cccc2c1sc1ccccc12]

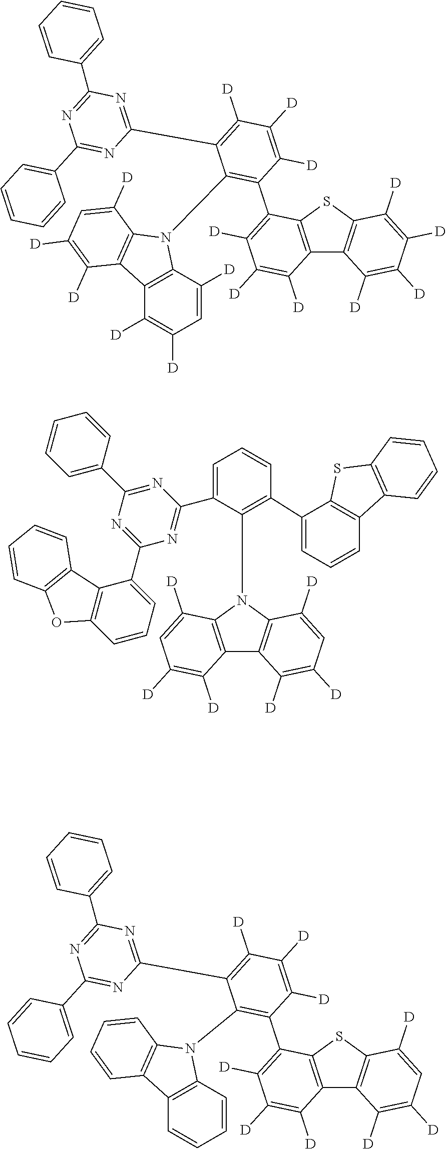 [SMILES: [2H]c1cc([2H])c2c(c1[2H])c1c([2H])c([2H])cc([2H])c1n2-c1c(-c2nc(-c3ccccc3)nc(-c3ccccc3)n2)c([2H])c([2H])c([2H])c1-c1c([2H])c([2H])c([2H])c2c1sc1c([2H])c([2H])c([2H])c([2H])c12 ; [2H]c1cc([2H])c2c(c1[2H])c1c([2H])c([2H])cc([2H])c1n2-c1c(-c2nc(-c3ccccc3)nc(-c3cccc4oc5ccccc5c34)n2)cccc1-c1cccc2c1sc1ccccc12 ; [2H]c1cc([2H])c2sc3c(-c4c([2H])c([2H])c([2H])c(-c5nc(-c6ccccc6)nc(-c6ccccc6)n5)c4-n4c5ccccc5c5ccccc54)c([2H])c([2H])c([2H])c3c2c1[2H]]

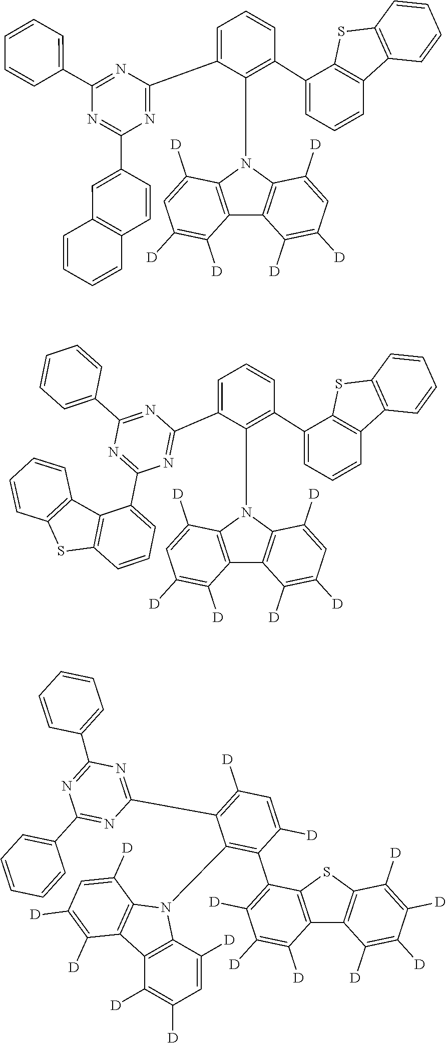 [SMILES: [2H]c1cc([2H])c2c(c1[2H])c1c([2H])c([2H])cc([2H])c1n2-c1c(-c2nc(-c3ccccc3)nc(-c3ccc4ccccc4c3)n2)cccc1-c1cccc2c1sc1ccccc12 ; [2H]c1cc([2H])c2c(c1[2H])c1c([2H])c([2H])cc([2H])c1n2-c1c(-c2nc(-c3ccccc3)nc(-c3cccc4sc5ccccc5c34)n2)cccc1-c1cccc2c1sc1ccccc12 ; [2H]c1cc([2H])c(-c2c([2H])c([2H])c([2H])c3c2sc2c([2H])c([2H])c([2H])c([2H])c23)c(-n2c3c([2H])cc([2H])c([2H])c3c3c([2H])c([2H])cc([2H])c32)c1-c1nc(-c2ccccc2)nc(-c2ccccc2)n1]

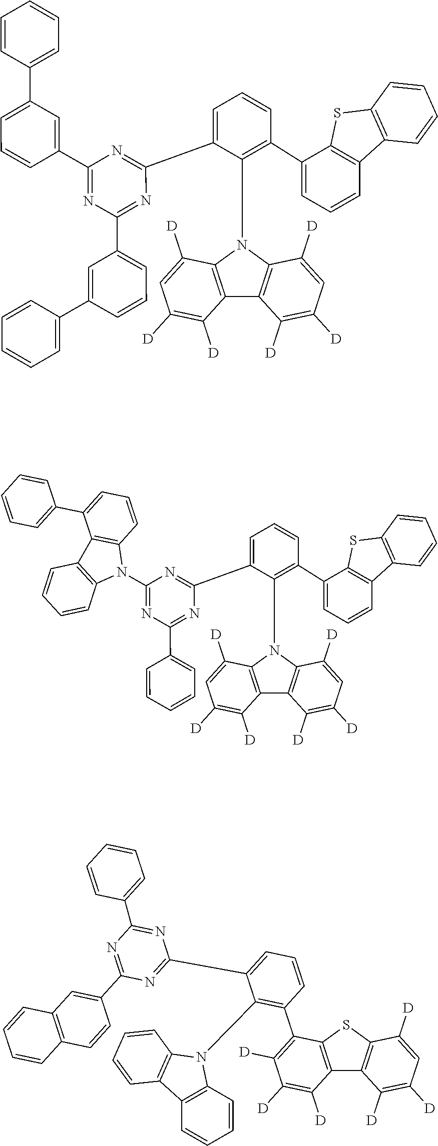 [SMILES: [2H]c1cc([2H])c2c(c1[2H])c1c([2H])c([2H])cc([2H])c1n2-c1c(-c2nc(-c3cccc(-c4ccccc4)c3)nc(-c3cccc(-c4ccccc4)c3)n2)cccc1-c1cccc2c1sc1ccccc12 ; [2H]c1cc([2H])c2c(c1[2H])c1c([2H])c([2H])cc([2H])c1n2-c1c(-c2nc(-c3ccccc3)nc(-n3c4ccccc4c4c(-c5ccccc5)cccc43)n2)cccc1-c1cccc2c1sc1ccccc12 ; [2H]c1cc([2H])c2sc3c(-c4cccc(-c5nc(-c6ccccc6)nc(-c6ccc7ccccc7c6)n5)c4-n4c5ccccc5c5ccccc54)c([2H])c([2H])c([2H])c3c2c1[2H]]

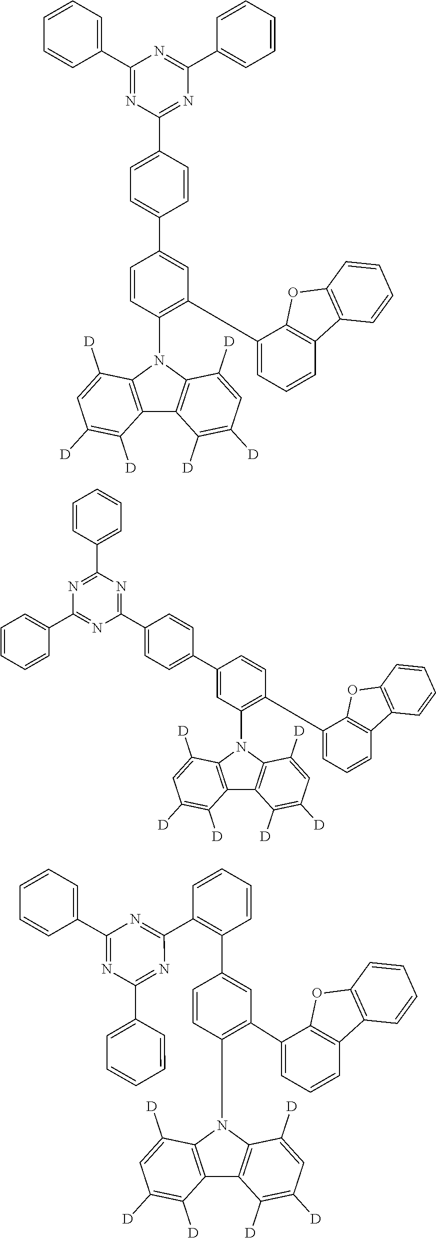

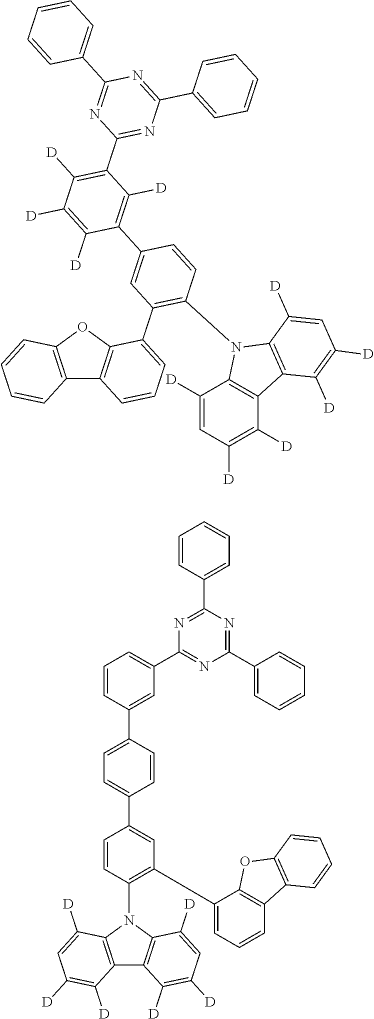 [SMILES: [2H]c1cc([2H])c2c(c1[2H])c1c([2H])c([2H])cc([2H])c1n2-c1ccc(-c2c([2H])c([2H])c([2H])c(-c3nc(-c4ccccc4)nc(-c4ccccc4)n3)c2[2H])cc1-c1cccc2c1oc1ccccc12 ; [2H]c1cc([2H])c2c(c1[2H])c1c([2H])c([2H])cc([2H])c1n2-c1ccc(-c2ccc(-c3cccc(-c4nc(-c5ccccc5)nc(-c5ccccc5)n4)c3)cc2)cc1-c1cccc2c1oc1ccccc12]

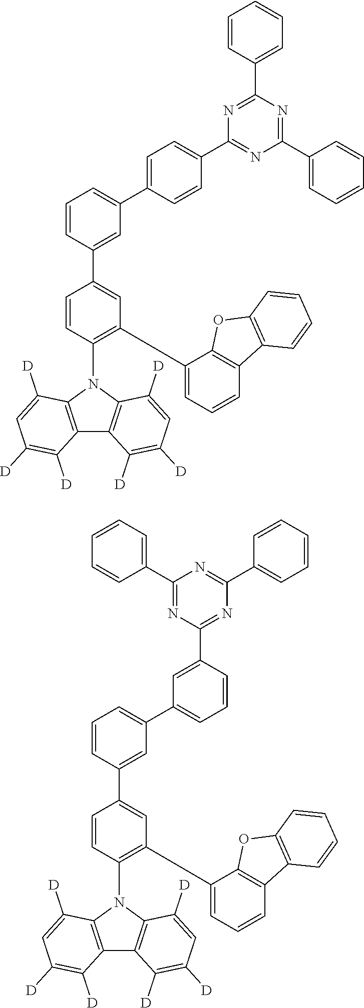

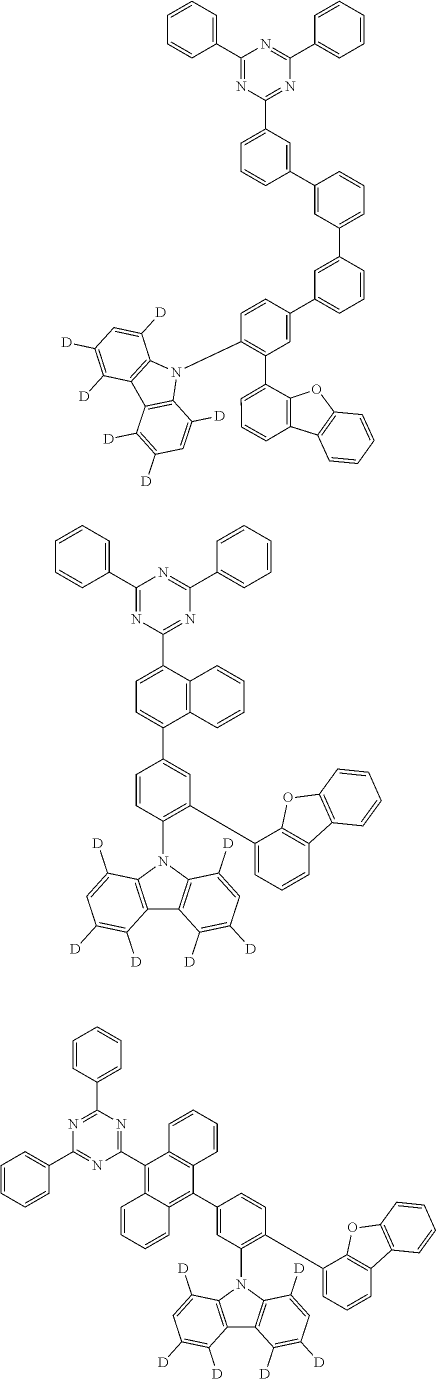 [SMILES: [2H]c1cc([2H])c2c(c1[2H])c1c([2H])c([2H])cc([2H])c1n2-c1ccc(-c2cccc(-c3cccc(-c4cccc(-c5nc(-c6ccccc6)nc(-c6ccccc6)n5)c4)c3)c2)cc1-c1cccc2c1oc1ccccc12 ; [2H]c1cc([2H])c2c(c1[2H])c1c([2H])c([2H])cc([2H])c1n2-c1ccc(-c2ccc(-c3nc(-c4ccccc4)nc(-c4ccccc4)n3)c3ccccc23)cc1-c1cccc2c1oc1ccccc12 ; [2H]c1cc([2H])c2c(c1[2H])c1c([2H])c([2H])cc([2H])c1n2-c1cc(-c2c3ccccc3c(-c3nc(-c4ccccc4)nc(-c4ccccc4)n3)c3ccccc23)ccc1-c1cccc2c1oc1ccccc12]

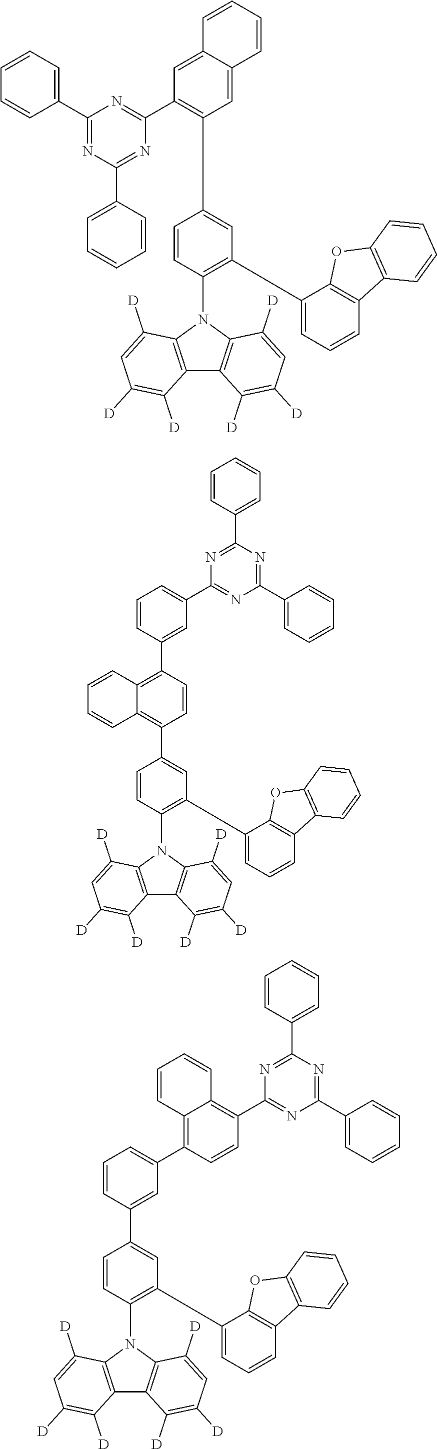

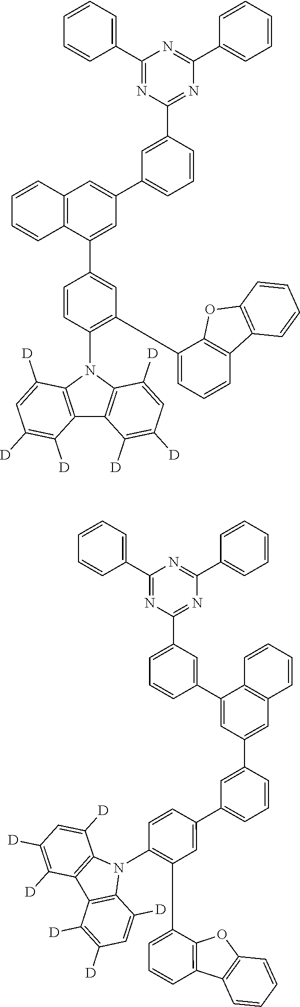 [SMILES: [2H]c1cc([2H])c2c(c1[2H])c1c([2H])c([2H])cc([2H])c1n2-c1ccc(-c2cc(-c3cccc(-c4nc(-c5ccccc5)nc(-c5ccccc5)n4)c3)cc3ccccc23)cc1-c1cccc2c1oc1ccccc12 ; [2H]c1cc([2H])c2c(c1[2H])c1c([2H])c([2H])cc([2H])c1n2-c1ccc(-c2cccc(-c3cc(-c4cccc(-c5nc(-c6ccccc6)nc(-c6ccccc6)n5)c4)c4ccccc4c3)c2)cc1-c1cccc2c1oc1ccccc12]

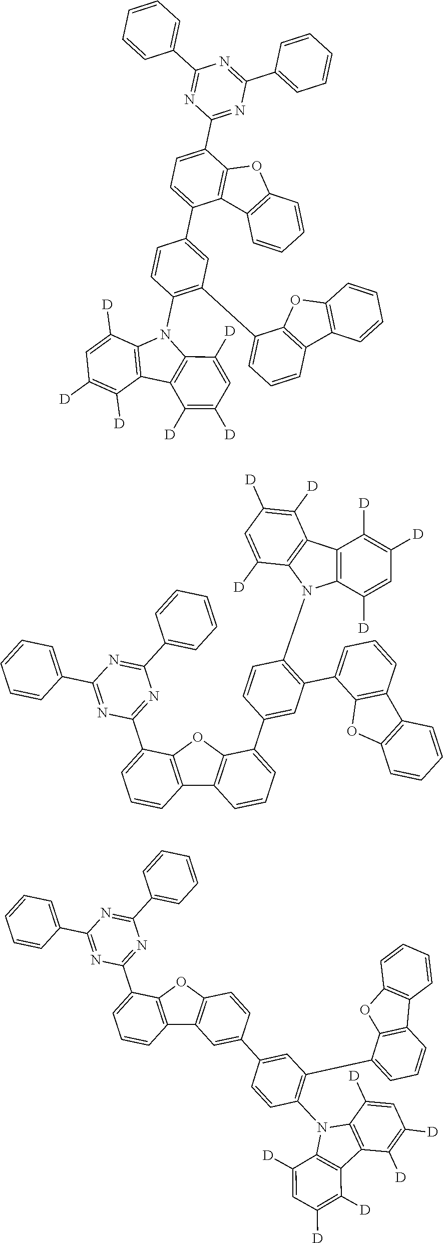 [SMILES: [2H]c1cc([2H])c2c(c1[2H])c1c([2H])c([2H])cc([2H])c1n2-c1ccc(-c2ccc(-c3nc(-c4ccccc4)nc(-c4ccccc4)n3)c3oc4ccccc4c23)cc1-c1cccc2c1oc1ccccc12 ; [2H]c1cc([2H])c2c(c1[2H])c1c([2H])c([2H])cc([2H])c1n2-c1ccc(-c2cccc3c2oc2c(-c4nc(-c5ccccc5)nc(-c5ccccc5)n4)cccc23)cc1-c1cccc2c1oc1ccccc12 ; [2H]c1cc([2H])c2c(c1[2H])c1c([2H])c([2H])cc([2H])c1n2-c1ccc(-c2ccc3oc4c(-c5nc(-c6ccccc6)nc(-c6ccccc6)n5)cccc4c3c2)cc1-c1cccc2c1oc1ccccc12]

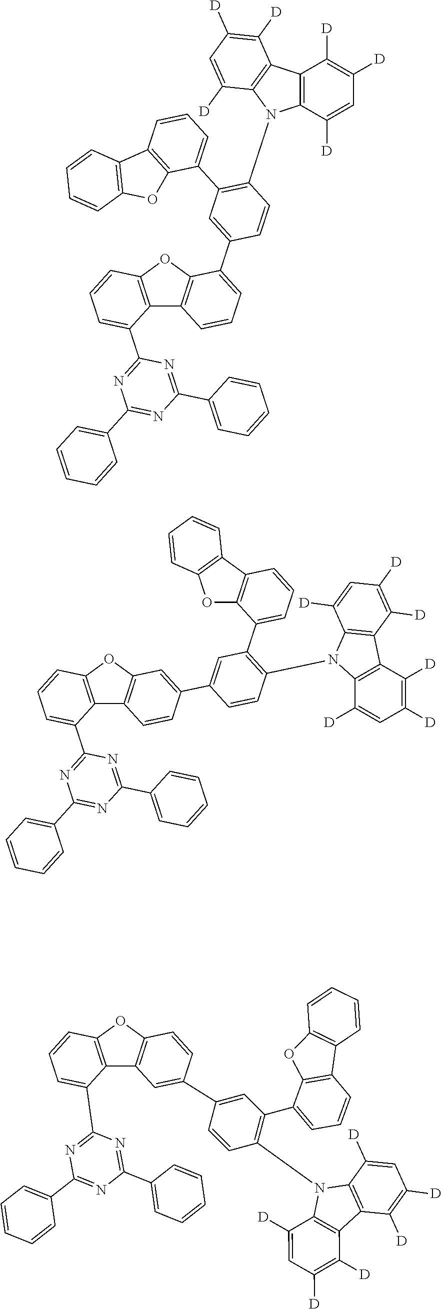 [SMILES: [2H]c1cc([2H])c2c(c1[2H])c1c([2H])c([2H])cc([2H])c1n2-c1ccc(-c2cccc3c2oc2cccc(-c4nc(-c5ccccc5)nc(-c5ccccc5)n4)c23)cc1-c1cccc2c1oc1ccccc12 ; [2H]c1cc([2H])c2c(c1[2H])c1c([2H])c([2H])cc([2H])c1n2-c1ccc(-c2ccc3c(c2)oc2cccc(-c4nc(-c5ccccc5)nc(-c5ccccc5)n4)c23)cc1-c1cccc2c1oc1ccccc12 ; [2H]c1cc([2H])c2c(c1[2H])c1c([2H])c([2H])cc([2H])c1n2-c1ccc(-c2ccc3oc4cccc(-c5nc(-c6ccccc6)nc(-c6ccccc6)n5)c4c3c2)cc1-c1cccc2c1oc1ccccc12]

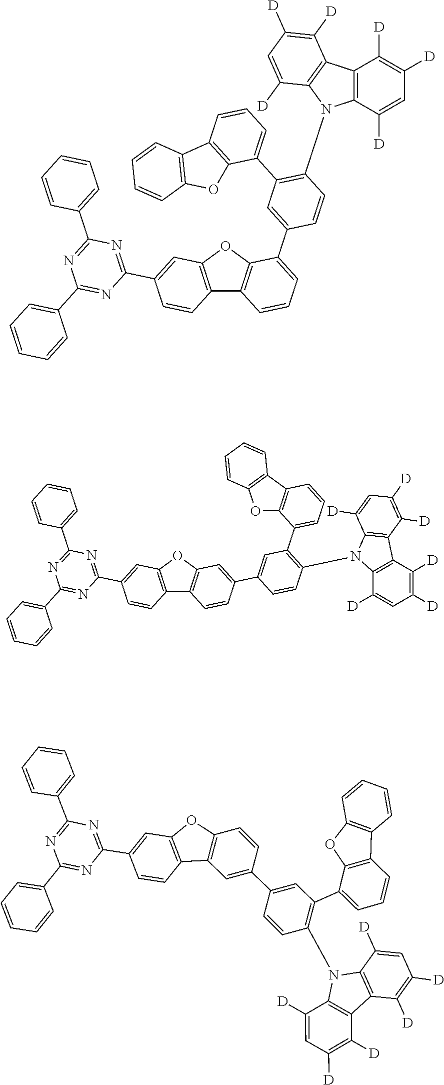 [SMILES: [2H]c1cc([2H])c2c(c1[2H])c1c([2H])c([2H])cc([2H])c1n2-c1ccc(-c2cccc3c2oc2cc(-c4nc(-c5ccccc5)nc(-c5ccccc5)n4)ccc23)cc1-c1cccc2c1oc1ccccc12 ; [2H]c1cc([2H])c2c(c1[2H])c1c([2H])c([2H])cc([2H])c1n2-c1ccc(-c2ccc3c(c2)oc2cc(-c4nc(-c5ccccc5)nc(-c5ccccc5)n4)ccc23)cc1-c1cccc2c1oc1ccccc12 ; [2H]c1cc([2H])c2c(c1[2H])c1c([2H])c([2H])cc([2H])c1n2-c1ccc(-c2ccc3oc4cc(-c5nc(-c6ccccc6)nc(-c6ccccc6)n5)ccc4c3c2)cc1-c1cccc2c1oc1ccccc12]

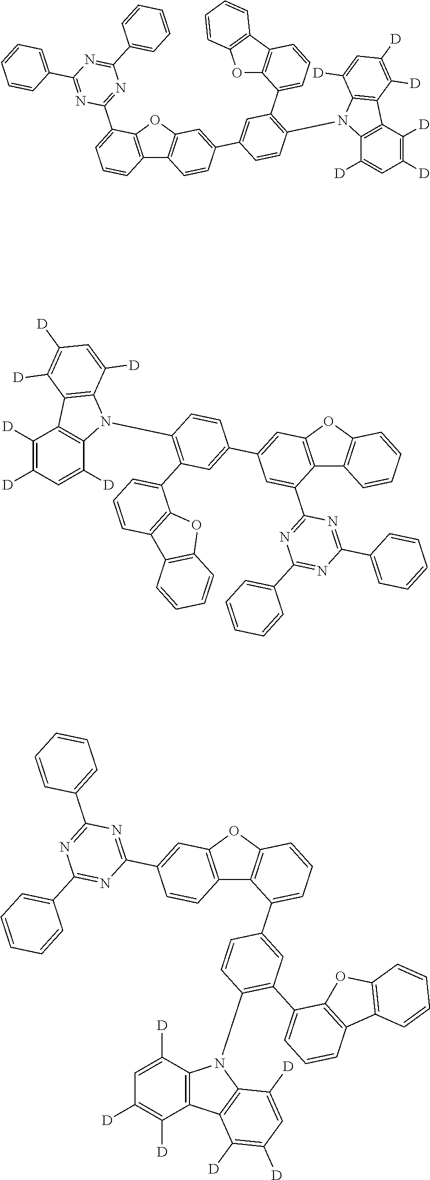 [SMILES: [2H]c1cc([2H])c2c(c1[2H])c1c([2H])c([2H])cc([2H])c1n2-c1ccc(-c2ccc3c(c2)oc2c(-c4nc(-c5ccccc5)nc(-c5ccccc5)n4)cccc23)cc1-c1cccc2c1oc1ccccc12 ; [2H]c1cc([2H])c2c(c1[2H])c1c([2H])c([2H])cc([2H])c1n2-c1ccc(-c2cc(-c3nc(-c4ccccc4)nc(-c4ccccc4)n3)c3c(c2)oc2ccccc23)cc1-c1cccc2c1oc1ccccc12 ; [2H]c1cc([2H])c2c(c1[2H])c1c([2H])c([2H])cc([2H])c1n2-c1ccc(-c2cccc3oc4cc(-c5nc(-c6ccccc6)nc(-c6ccccc6)n5)ccc4c23)cc1-c1cccc2c1oc1ccccc12]

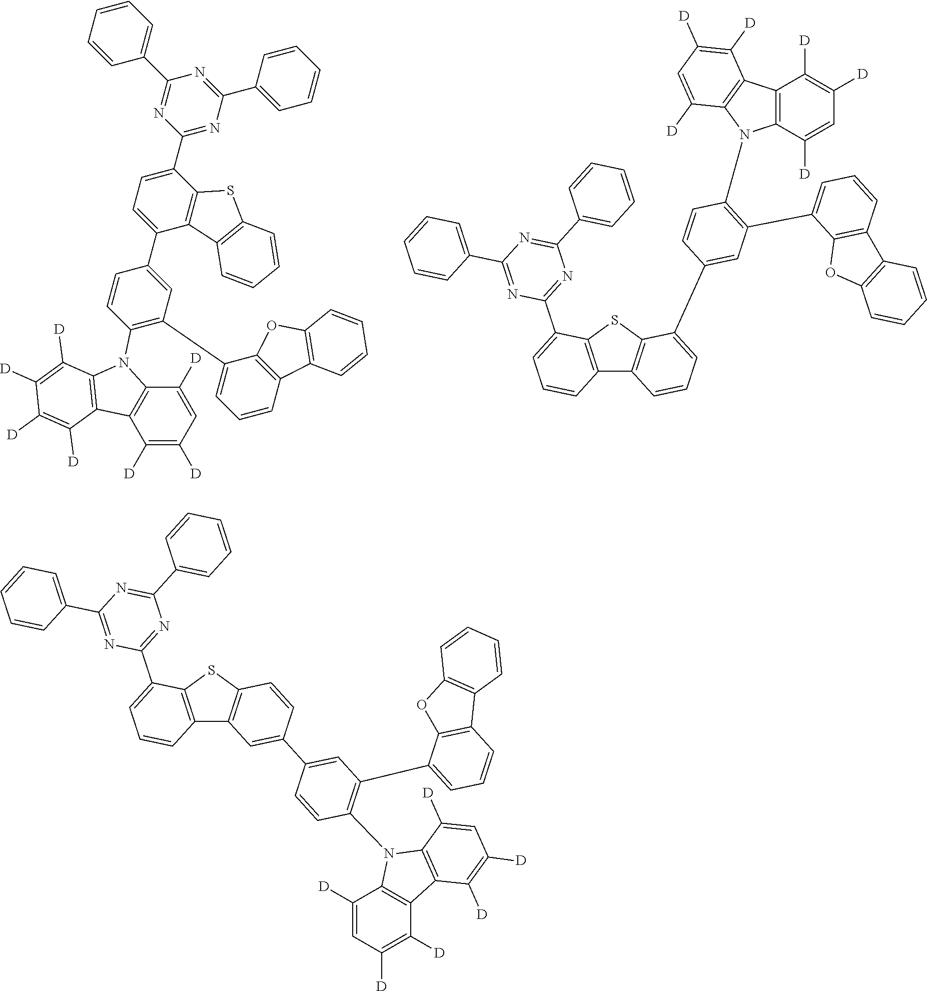

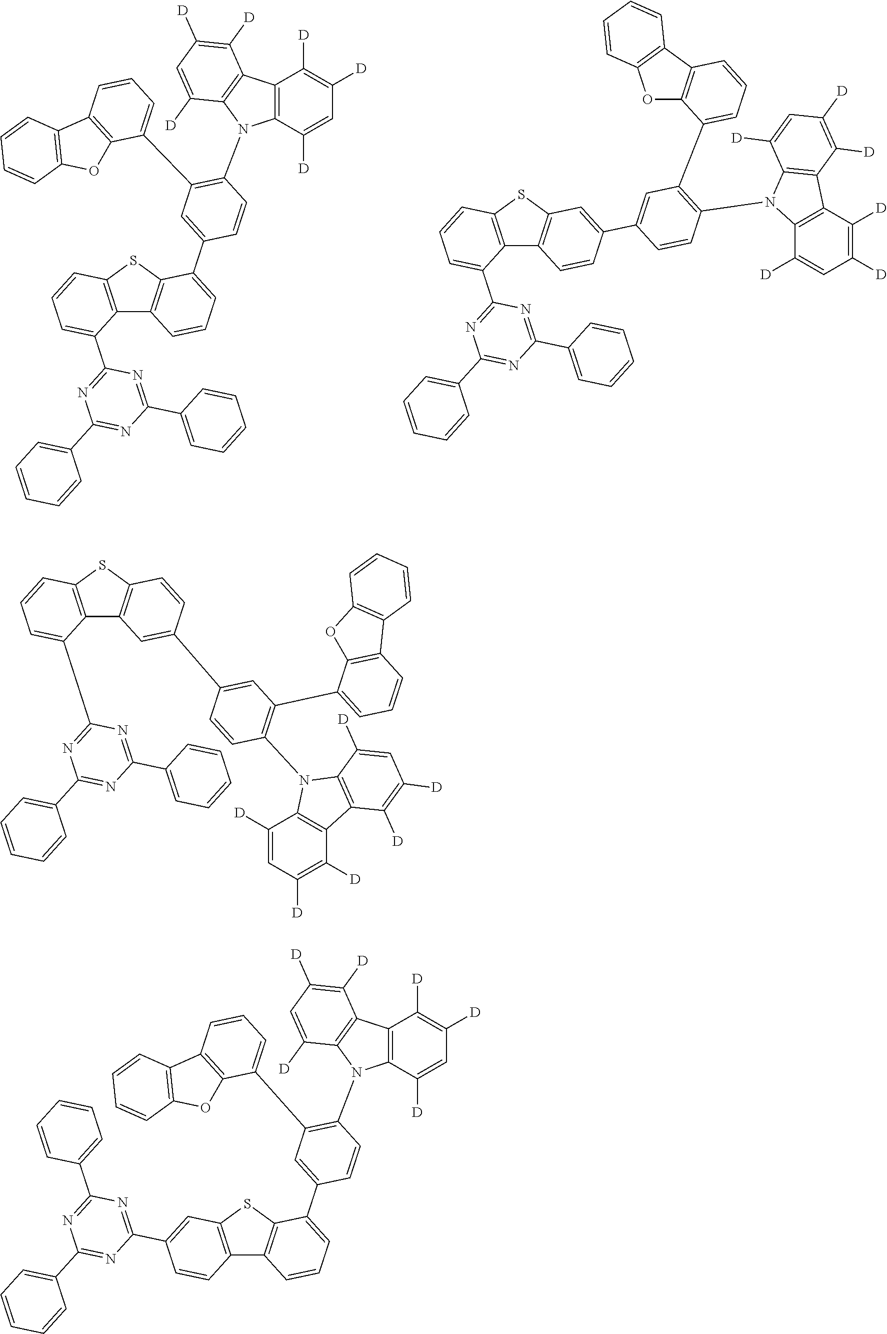

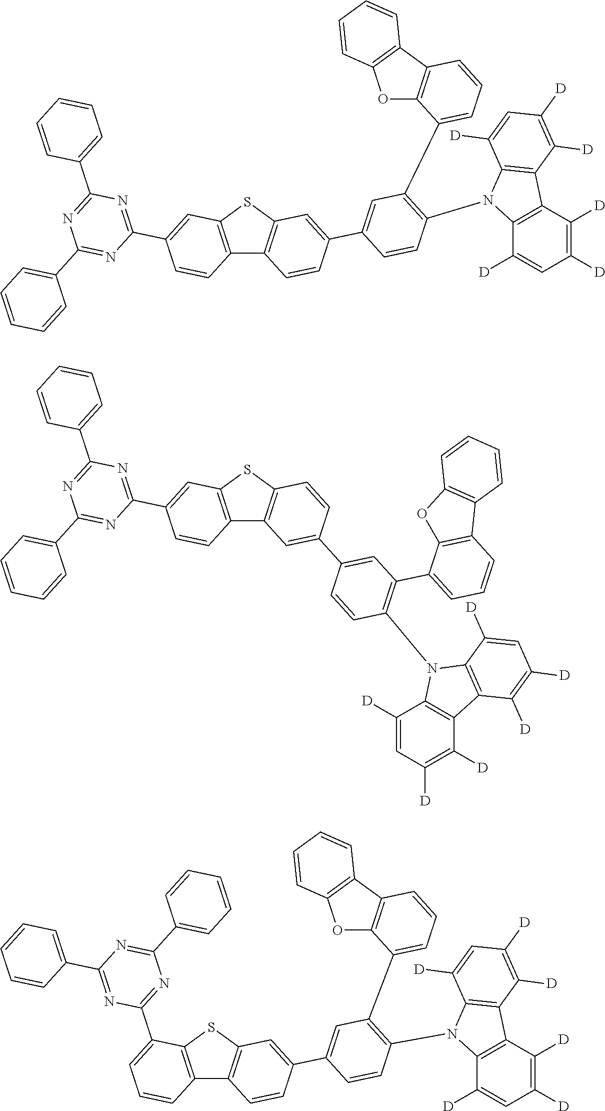

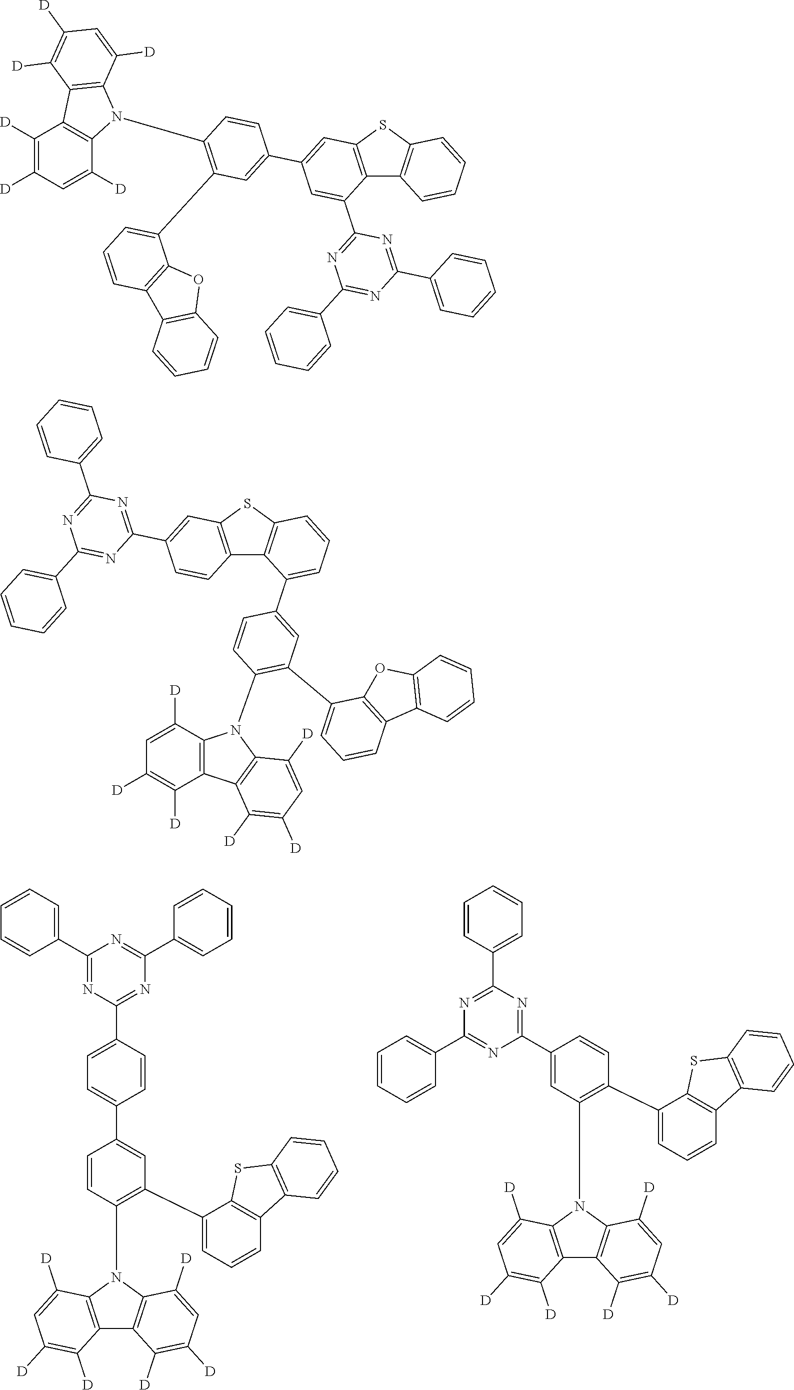

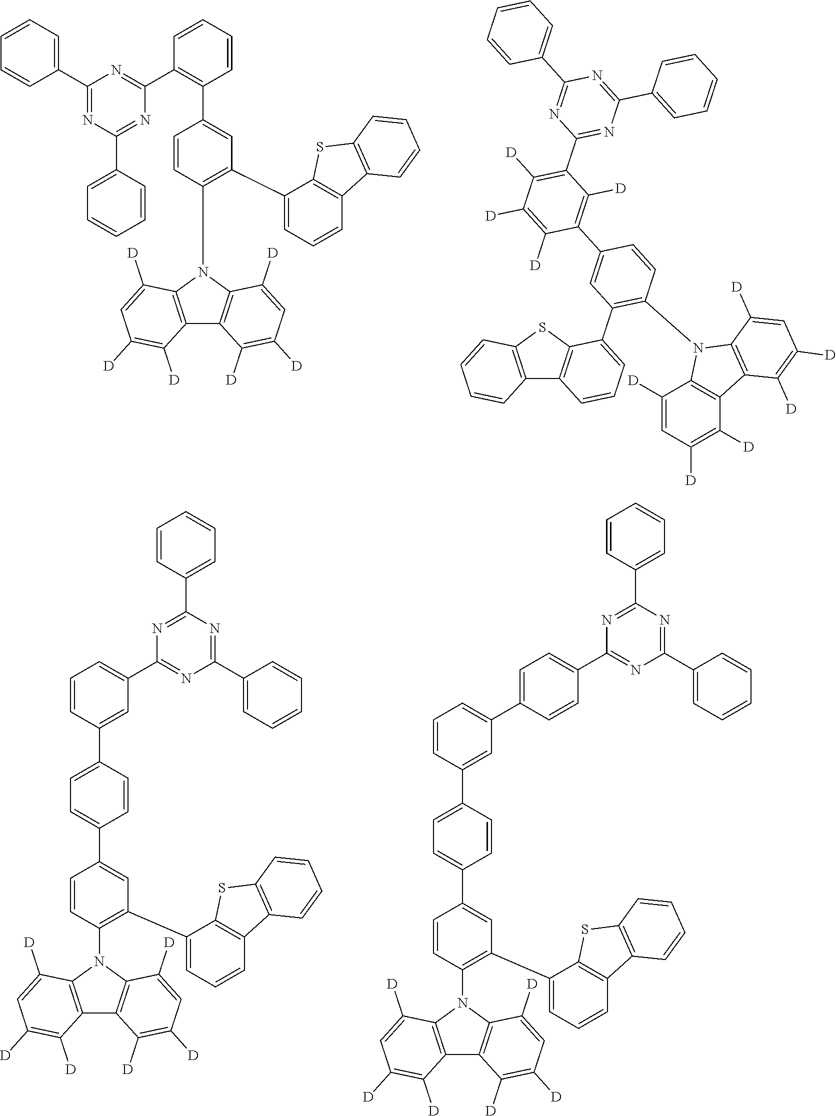

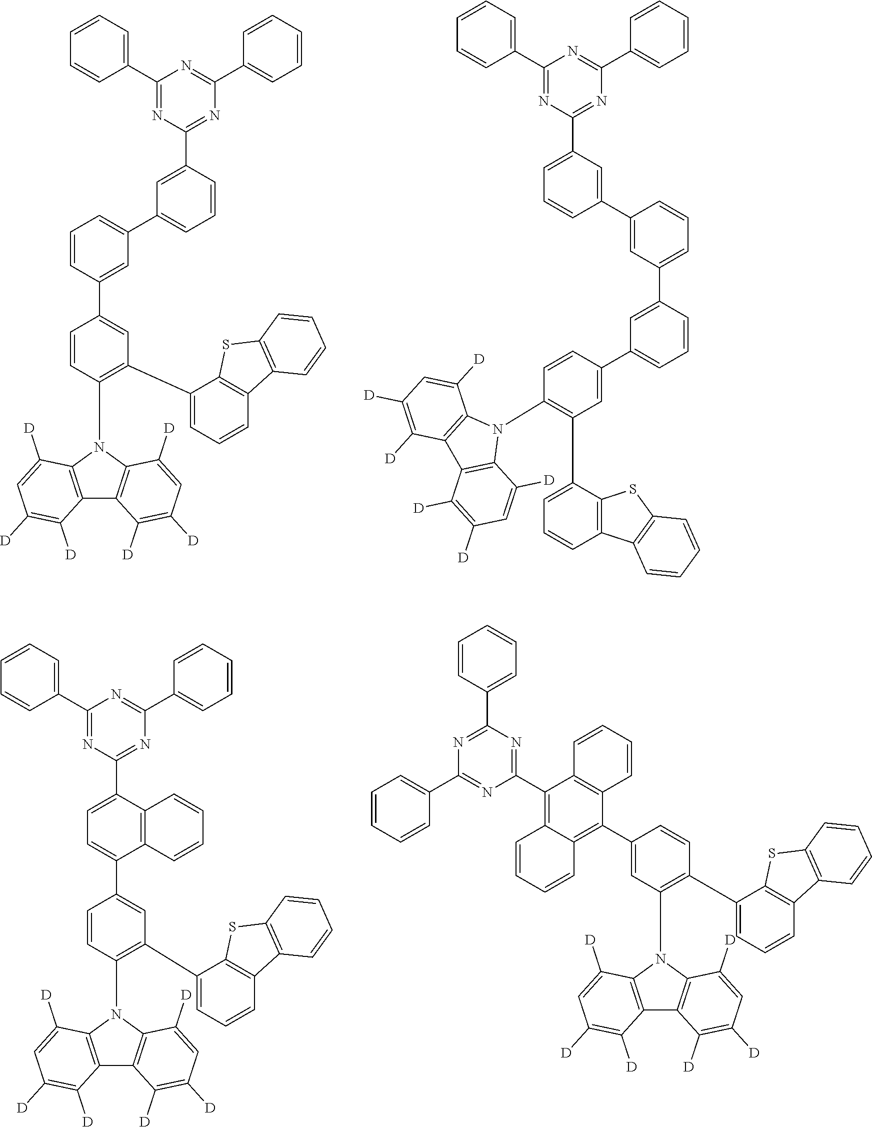

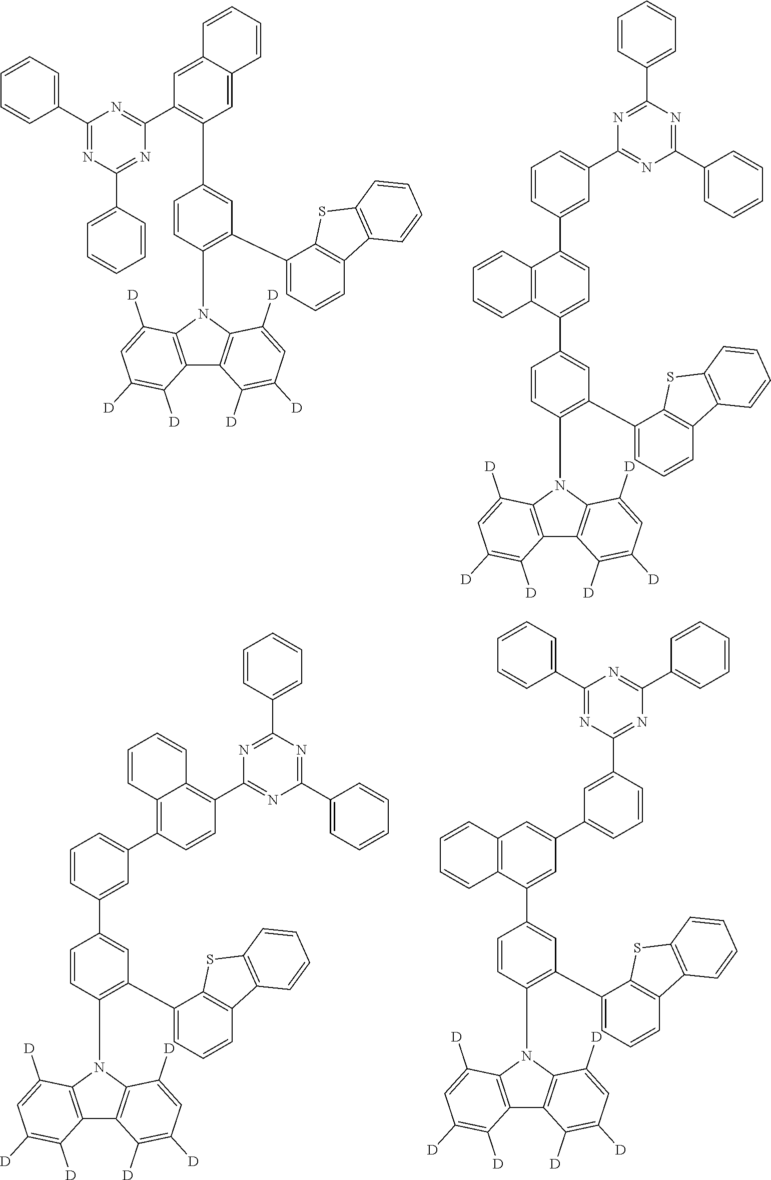

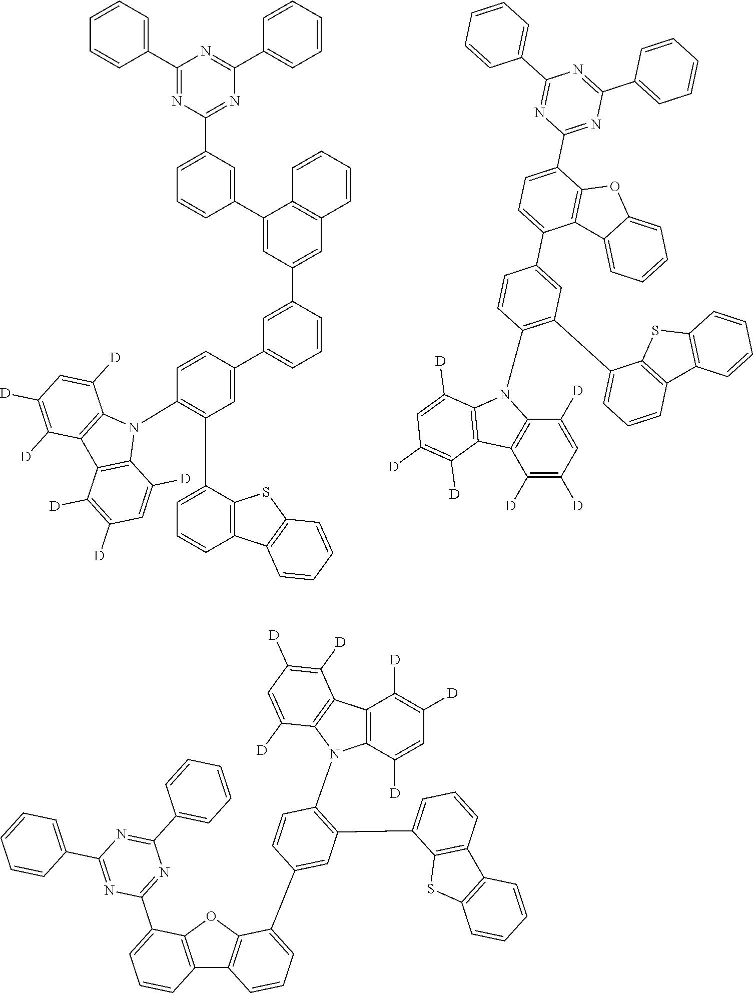 [SMILES: [2H]c1cc([2H])c2c(c1[2H])c1c([2H])c([2H])cc([2H])c1n2-c1ccc(-c2cccc(-c3cc(-c4cccc(-c5nc(-c6ccccc6)nc(-c6ccccc6)n5)c4)c4ccccc4c3)c2)cc1-c1cccc2c1sc1ccccc12 ; [2H]c1cc([2H])c2c(c1[2H])c1c([2H])c([2H])cc([2H])c1n2-c1ccc(-c2ccc(-c3nc(-c4ccccc4)nc(-c4ccccc4)n3)c3oc4ccccc4c23)cc1-c1cccc2c1sc1ccccc12 ; [2H]c1cc([2H])c2c(c1[2H])c1c([2H])c([2H])cc([2H])c1n2-c1ccc(-c2cccc3c2oc2c(-c4nc(-c5ccccc5)nc(-c5ccccc5)n4)cccc23)cc1-c1cccc2c1sc1ccccc12]

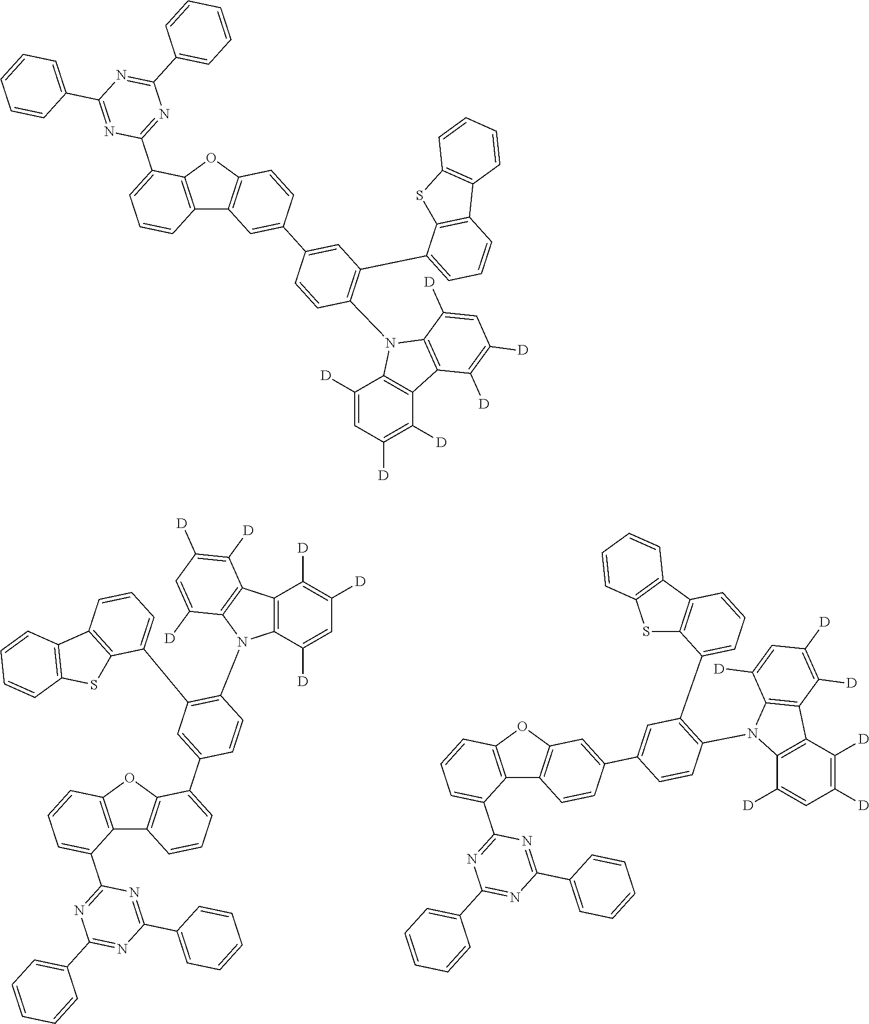 [SMILES: [2H]c1cc([2H])c2c(c1[2H])c1c([2H])c([2H])cc([2H])c1n2-c1ccc(-c2ccc3oc4c(-c5nc(-c6ccccc6)nc(-c6ccccc6)n5)cccc4c3c2)cc1-c1cccc2c1sc1ccccc12 ; [2H]c1cc([2H])c2c(c1[2H])c1c([2H])c([2H])cc([2H])c1n2-c1ccc(-c2cccc3c2oc2cccc(-c4nc(-c5ccccc5)nc(-c5ccccc5)n4)c23)cc1-c1cccc2c1sc1ccccc12 ; [2H]c1cc([2H])c2c(c1[2H])c1c([2H])c([2H])cc([2H])c1n2-c1ccc(-c2ccc3c(c2)oc2cccc(-c4nc(-c5ccccc5)nc(-c5ccccc5)n4)c23)cc1-c1cccc2c1sc1ccccc12]

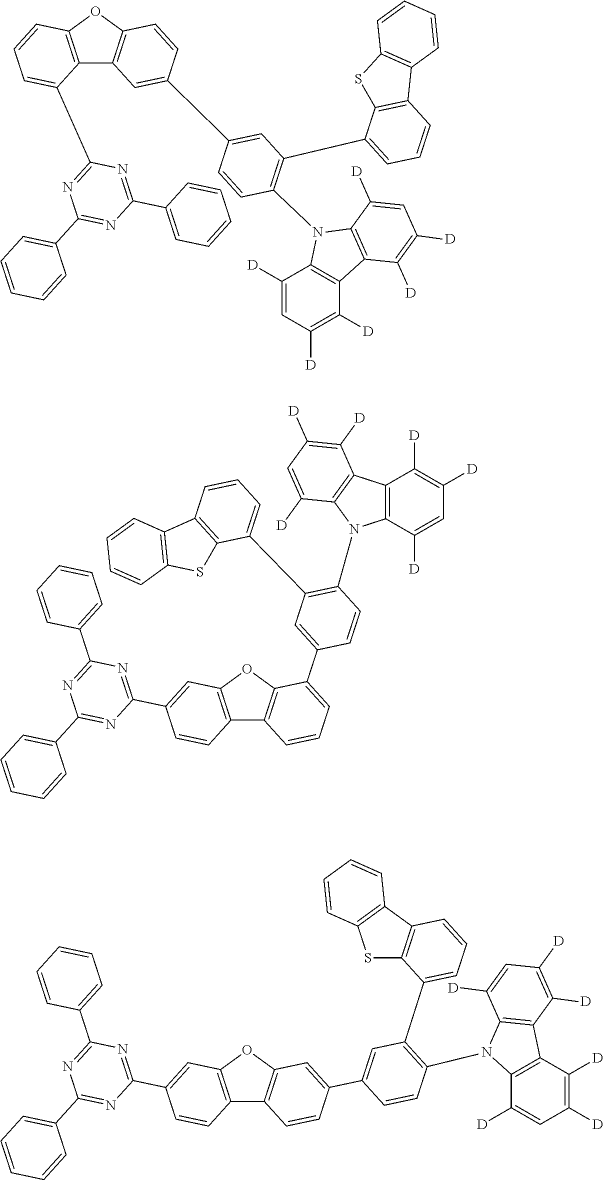 [SMILES: [2H]c1cc([2H])c2c(c1[2H])c1c([2H])c([2H])cc([2H])c1n2-c1ccc(-c2ccc3oc4cccc(-c5nc(-c6ccccc6)nc(-c6ccccc6)n5)c4c3c2)cc1-c1cccc2c1sc1ccccc12 ; [2H]c1cc([2H])c2c(c1[2H])c1c([2H])c([2H])cc([2H])c1n2-c1ccc(-c2cccc3c2oc2cc(-c4nc(-c5ccccc5)nc(-c5ccccc5)n4)ccc23)cc1-c1cccc2c1sc1ccccc12 ; [2H]c1cc([2H])c2c(c1[2H])c1c([2H])c([2H])cc([2H])c1n2-c1ccc(-c2ccc3c(c2)oc2cc(-c4nc(-c5ccccc5)nc(-c5ccccc5)n4)ccc23)cc1-c1cccc2c1sc1ccccc12]

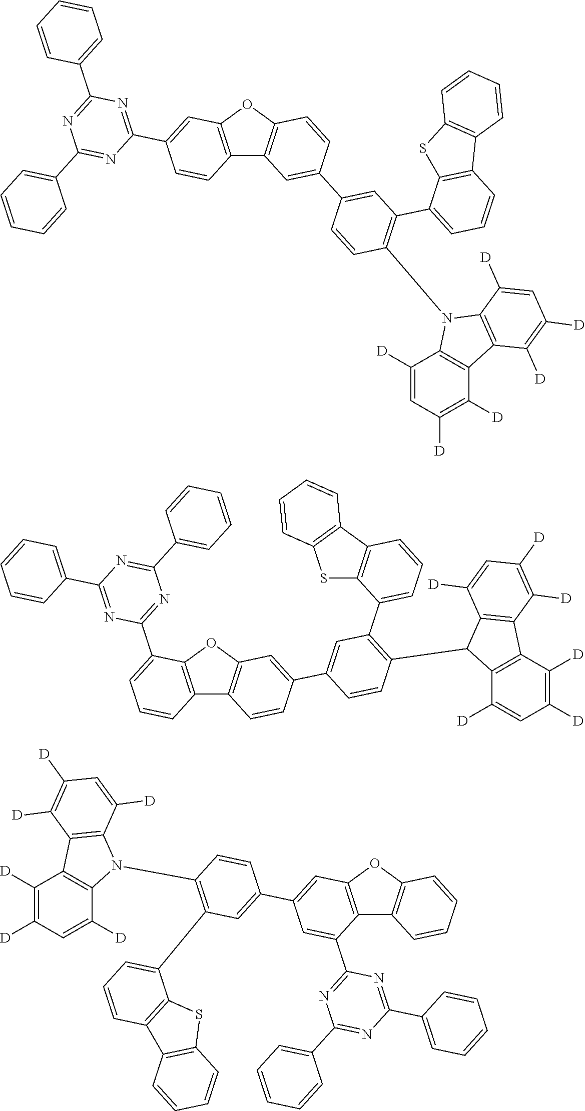 [SMILES: [2H]c1cc([2H])c2c(c1[2H])c1c([2H])c([2H])cc([2H])c1n2-c1ccc(-c2ccc3oc4cc(-c5nc(-c6ccccc6)nc(-c6ccccc6)n5)ccc4c3c2)cc1-c1cccc2c1sc1ccccc12 ; [2H]c1cc([2H])c2c(c1[2H])-c1c([2H])c([2H])cc([2H])c1C2c1ccc(-c2ccc3c(c2)oc2c(-c4nc(-c5ccccc5)nc(-c5ccccc5)n4)cccc23)cc1-c1cccc2c1sc1ccccc12 ; [2H]c1cc([2H])c2c(c1[2H])c1c([2H])c([2H])cc([2H])c1n2-c1ccc(-c2cc(-c3nc(-c4ccccc4)nc(-c4ccccc4)n3)c3c(c2)oc2ccccc23)cc1-c1cccc2c1sc1ccccc12]

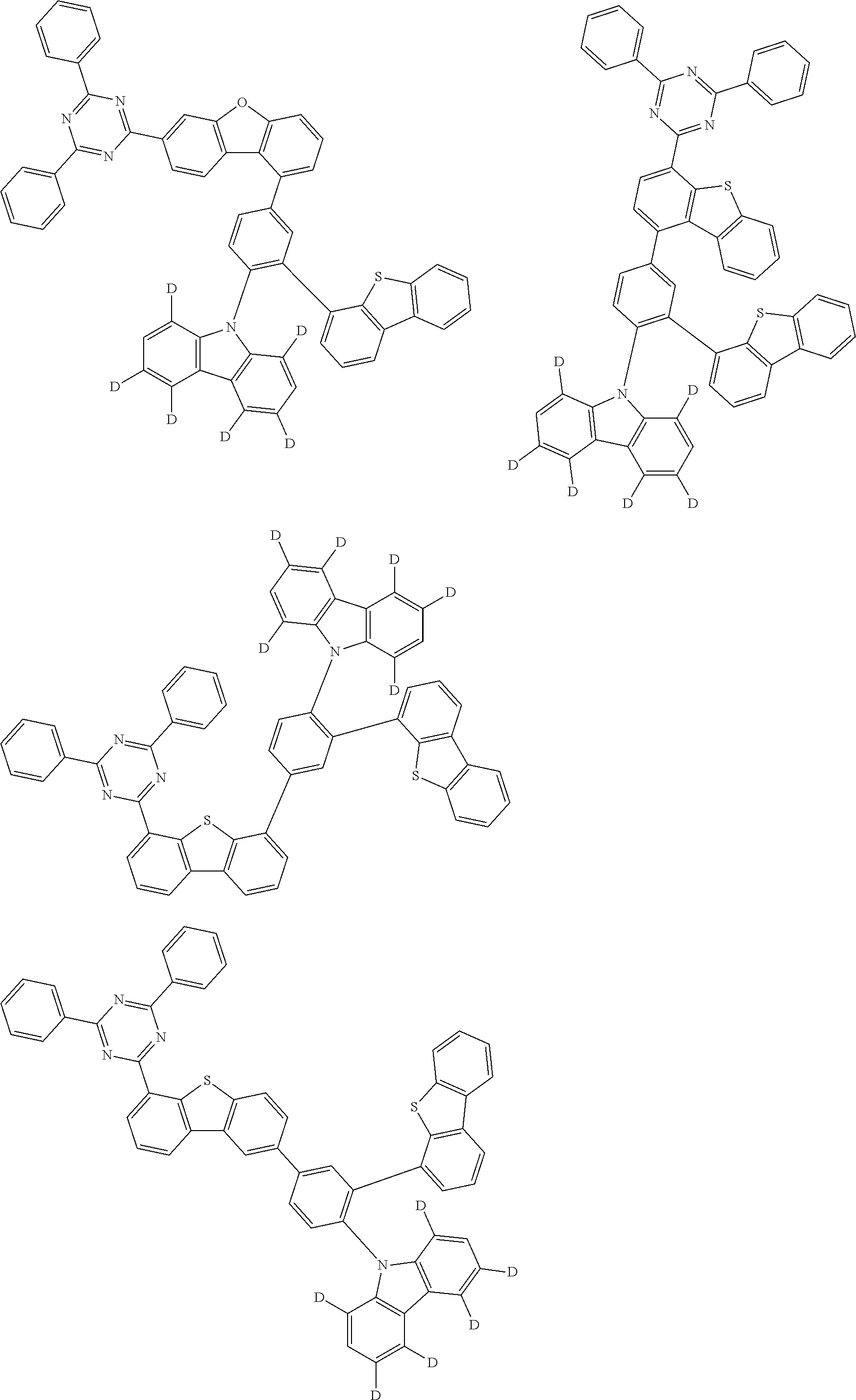

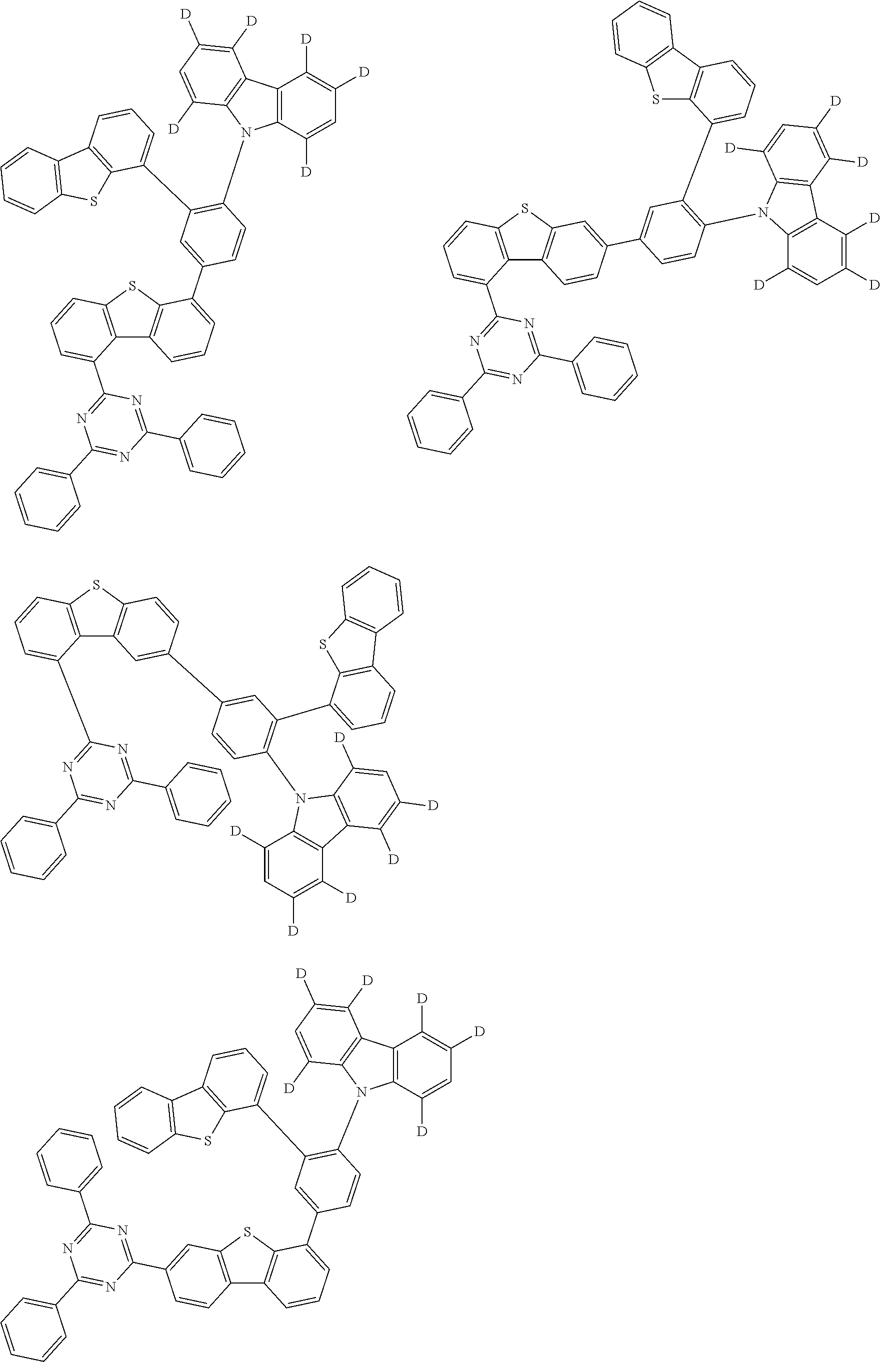

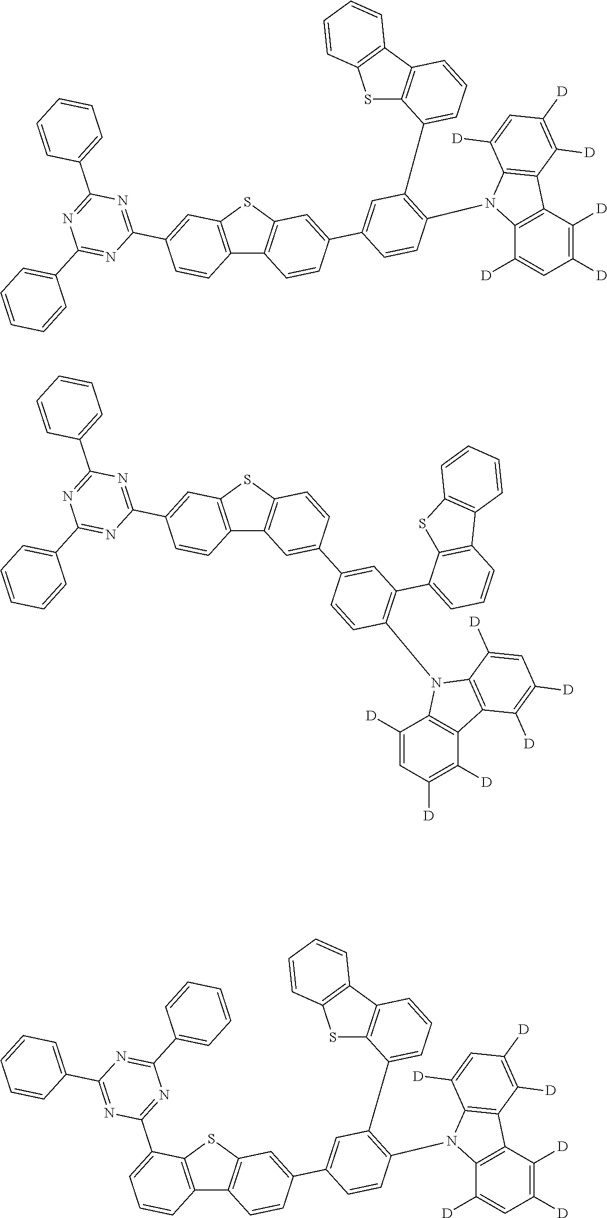 [SMILES: [2H]c1cc([2H])c2c(c1[2H])c1c([2H])c([2H])cc([2H])c1n2-c1ccc(-c2ccc3c(c2)sc2cc(-c4nc(-c5ccccc5)nc(-c5ccccc5)n4)ccc23)cc1-c1cccc2c1sc1ccccc12 ; [2H]c1cc([2H])c2c(c1[2H])c1c([2H])c([2H])cc([2H])c1n2-c1ccc(-c2ccc3sc4cc(-c5nc(-c6ccccc6)nc(-c6ccccc6)n5)ccc4c3c2)cc1-c1cccc2c1sc1ccccc12 ; [2H]c1cc([2H])c2c(c1[2H])c1c([2H])c([2H])cc([2H])c1n2-c1ccc(-c2ccc3c(c2)sc2c(-c4nc(-c5ccccc5)nc(-c5ccccc5)n4)cccc23)cc1-c1cccc2c1sc1ccccc12]

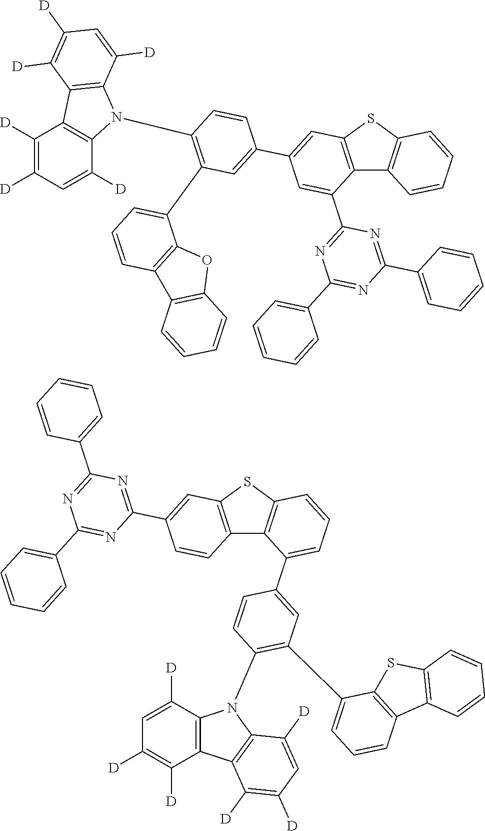 [SMILES: [2H]c1cc([2H])c2c(c1[2H])c1c([2H])c([2H])cc([2H])c1n2-c1ccc(-c2cc(-c3nc(-c4ccccc4)nc(-c4ccccc4)n3)c3c(c2)sc2ccccc23)cc1-c1cccc2c1oc1ccccc12 ; [2H]c1cc([2H])c2c(c1[2H])c1c([2H])c([2H])cc([2H])c1n2-c1ccc(-c2cccc3sc4cc(-c5nc(-c6ccccc6)nc(-c6ccccc6)n5)ccc4c23)cc1-c1cccc2c1sc1ccccc12]

Meanwhile, the compound of Chemical Formula 1 can be prepared by the preparation method as shown in the following Reaction Scheme 1 as an example.

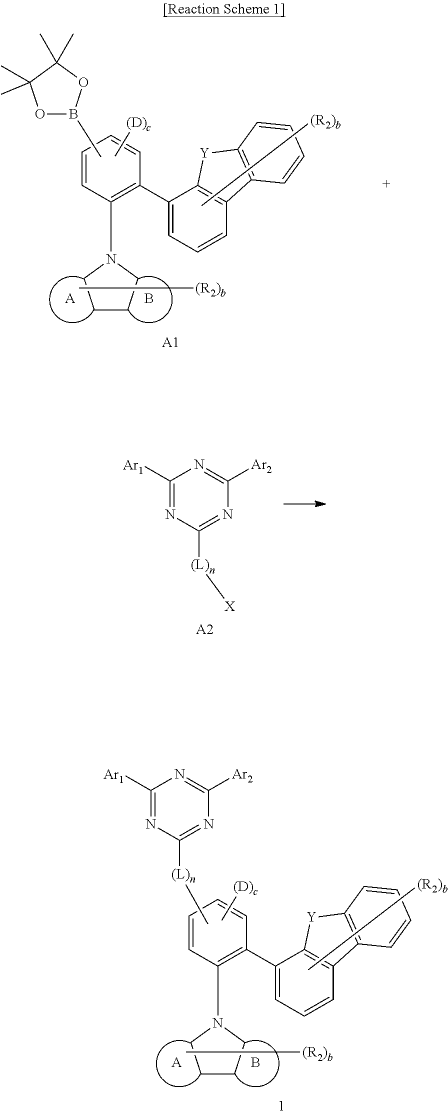

In Reaction Scheme 1, X is halogen, more preferably bromo or chloro, and the remaining substituents are the same as defined above.

Specifically, the compound of Chemical Formula 1 can be prepared from a Suzuki-coupling reaction of the reaction materials A1 and A2. At this time, the Suzuki-coupling reaction is preferably carried out in the presence of a palladium catalyst and a base, and a reactive group for the Suzuki-coupling reaction can be appropriately changed to a reactive group know in the art. Such a preparation method can be further embodied in the Preparation Examples described hereinafter.

### (Organic Light Emitting Device)

Meanwhile, the present disclosure provides an organic light emitting device comprising a compound of Chemical Formula 1. In one example, the present disclosure provides an organic light emitting device comprising: a first electrode; a second electrode that is opposite to the first electrode; and one or more organic material layers that are between the first electrode and the second electrode, wherein one or more layers of the one or more organic material layers includes the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure comprising a hole injection layer, a hole transport layer, a light emitting layer, an electron transport layer, an electron injection layer and the like as the organic material layer. However, the structure of the organic light emitting device is not limited thereto, and it can include a smaller number of organic layers.

In one embodiment, the organic material layer can include a light emitting layer, wherein the organic material layer including the compound can be a light emitting layer.

In another embodiment, the organic material layer can include a hole injection layer, a hole transport layer, a light emitting layer, an electron transport layer and an electron injection layer, wherein the organic material layer including the compound can be a light emitting layer or an electron transport layer.

In another embodiment, the organic material layer can include a hole injection layer, a hole transport layer, an electron blocking layer, a light emitting layer, an electron transport layer and an electron injection layer, wherein the organic material layer including the compound can be a light emitting layer or an electron transport layer.

In yet another embodiment, the organic material layer can include a hole injection layer, a hole transport layer, an electron blocking layer, a light emitting layer, a hole blocking layer, an electron transport layer and an electron injection layer, wherein the organic material layer including the compound can be a light emitting layer or an electron transport layer.

The organic material layer of the organic light emitting device of the present disclosure can have a single-layer structure, or it can have a multilayered structure in which two or more organic material layers are stacked. For example, the organic light emitting device of the present disclosure can have a structure further comprising a hole injection layer and a hole transport layer between the first electrode and the light emitting layer, and an electron transport layer and an electron injection layer between the light emitting layer and the second electrode, in addition to the light emitting layer, as the organic material layer. However, the structure of the organic light emitting device is not limited thereto, and it can include a smaller number of organic layers or a larger number of organic layers.

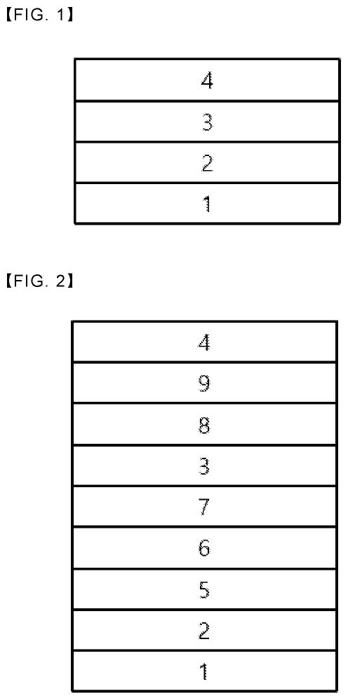

Further, the organic light emitting device according to the present disclosure can be a normal type organic light emitting device in which an anode, one or more organic material layers and a cathode are sequentially stacked on a substrate, wherein the first electrode is an anode, and the second electrode is a cathode. Further, the organic light emitting device according to the present disclosure can be an inverted type organic light emitting device in which a cathode, one or more organic material layers and an anode are sequentially stacked on a substrate, wherein the first electrode is a cathode and the second electrode is an anode. For example, the structure of the organic light emitting device according to one embodiment of the present disclosure is illustrated in FIGS. 1 and 2.

FIG. 1 shows an example of an organic light emitting device comprising a substrate 1, an anode 2, a light emitting layer 3, and a cathode 4. In such a structure, the compound of Chemical Formula 1 can be included in the light emitting layer.

FIG. 2 shows an example of an organic light emitting device comprising a substrate 1, an anode 2, a hole injection layer 5, a hole transport layer 6, an electron blocking layer 7, a light emitting layer 3, an electron transport layer 8, an electron injection layer 9, and a cathode 4. In such a structure, the compound of Chemical Formula 1 can be included in the light emitting layer.

The organic light emitting device according to the present disclosure can be manufactured by materials and methods known in the art, except that at least one of the organic material layers includes the compound of Chemical Formula 1. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO: Al or SnO₂: Sb; conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

Further, the hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to an electron injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

Further, the hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. The hole transport material includes an arylamine-based organic material, a conductive polymer, a block copolymer in which a conjugate portion and a non-conjugate portion are present together, and the like, but are not limited thereto.

Further, the electron blocking layer refers to a layer which is formed on the hole transport layer, preferably provided in contact with the light emitting layer, and serves to adjust the hole mobility, prevent excessive movement of electrons, and increase the probability of hole-electron coupling, thereby improving the efficiency of the organic light emitting device. The electron blocking layer includes an electron blocking material, and examples of such electron blocking material can include an arylamine-based organic material or the like, but is not limited thereto.

Further, the light emitting layer can include a host material and a dopant material. The compound of Chemical Formula 1 can be used as such a host material. Further, the host material can further include a fused aromatic ring derivative, a heterocycle-containing compound or the like in addition to the compound of Chemical Formula 1. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

In one embodiment, the light emitting layer can further include a compound of the following Chemical Formula 2, in addition to the compound of Chemical Formula 1:

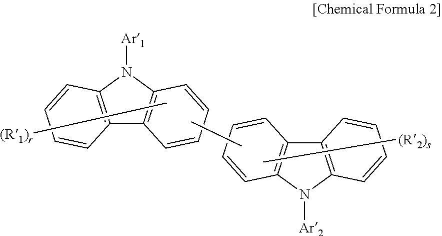

wherein, in Chemical Formula 2:
Ar′₁ and Ar′₂ are each independently a substituted or unsubstituted C₆₋₆₀ aryl or a substituted or unsubstituted C₂₋₆₀ heteroaryl containing one or more heteroatoms of N, O and S;
R′₁ and R′₂ are each independently deuterium, a C₁₋₆₀ alkyl, a C₆₋₆₀ aryl, or a C₂₋₆₀ heteroaryl containing one or more heteroatoms of N, O and S; and
r and s are each independently an integer of 0 to 7.

When the organic light emitting device further includes the compound of Chemical Formula 2, which can efficiently transfer holes to a dopant material from a host material for the light emitting layer, the recombination probability between holes and electrons in the light emitting layer increases with the compound of Chemical Formula 1 having excellent electron transport capability, thereby being capable of enhancing the efficiency and lifetime of the organic light emitting device.

According to one embodiment, the compound of Chemical Formula 2 can be Chemical Formula 2′:

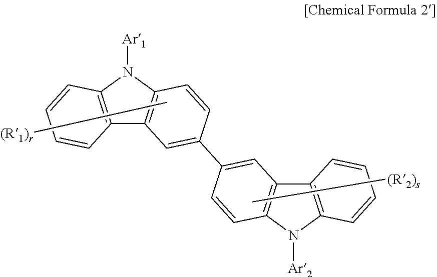

wherein, in Chemical Formula 2′,
Ar′₁, Ar′₂, R′₁, R′₂, r and s are as defined in Chemical Formula 2.

Further, in Chemical Formula 2, Ar′₁ and Ar′₂ are each independently a C₆₋₂₀ aryl, or a C₂₋₂₀ heteroaryl containing one heteroatom of N, O and S,
wherein Ar′₁ can be unsubstituted or substituted with one or more substituents selected from the group consisting of deuterium and a C₆₋₂₀ aryl.

For example, Ar′₁ and Ar′₂ can be each independently phenyl, biphenylyl, terphenylyl, naphthyl, dibenzofuranyl, or dibenzothiophenyl,
wherein Ar′₁ can be unsubstituted or substituted with one or more substituents selected from the group consisting of deuterium and a C₆₋₂₀ aryl.

At this time, at least one of Ar′₁ and Ar′₂ can be phenyl or biphenylyl.

Further, in Chemical Formula 2, R′₁ and R′₂ can be each independently hydrogen, deuterium, or a C₆₋₂₀ aryl.

For example, R′₁ and R′₂ can be each independently hydrogen, deuterium, or phenyl, but are not limited thereto.

Further, r and s, each indicating the number of R′₁ and R′₂, can be each independently 0, 1, 2, 3, 4, 5, 6, or 7.

More specifically, r and s can be each independently 0, 1, or 7.

For example, r+s can be 0 or 1.

Representative examples of the compound of Chemical Formula 2 are as follows:

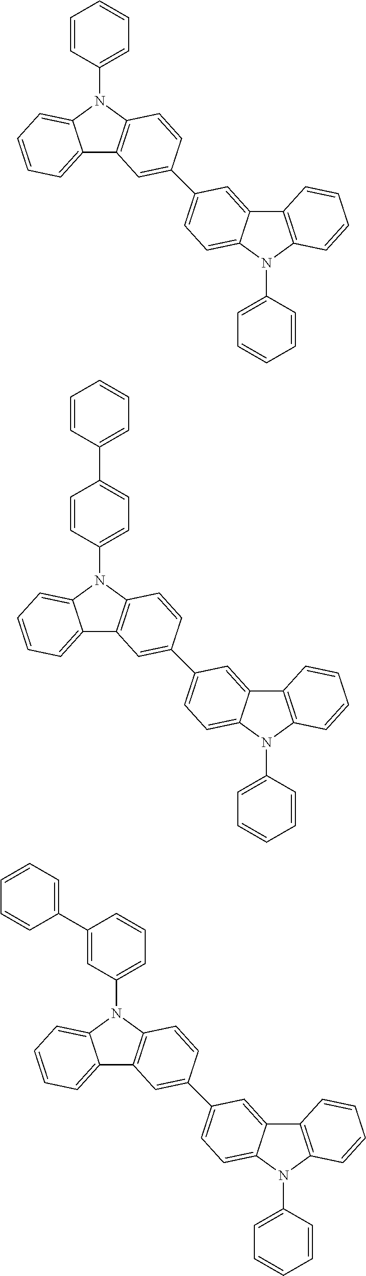 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

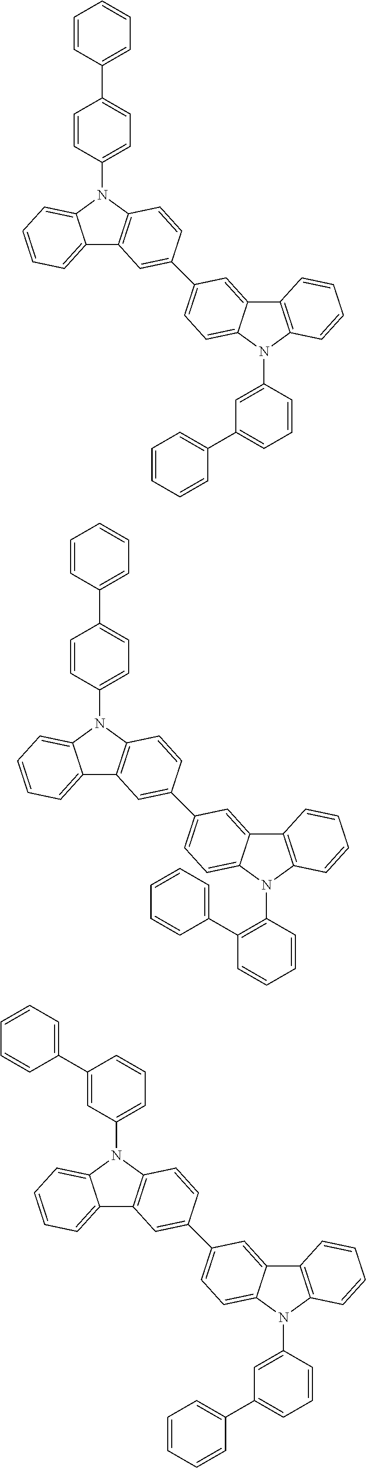 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

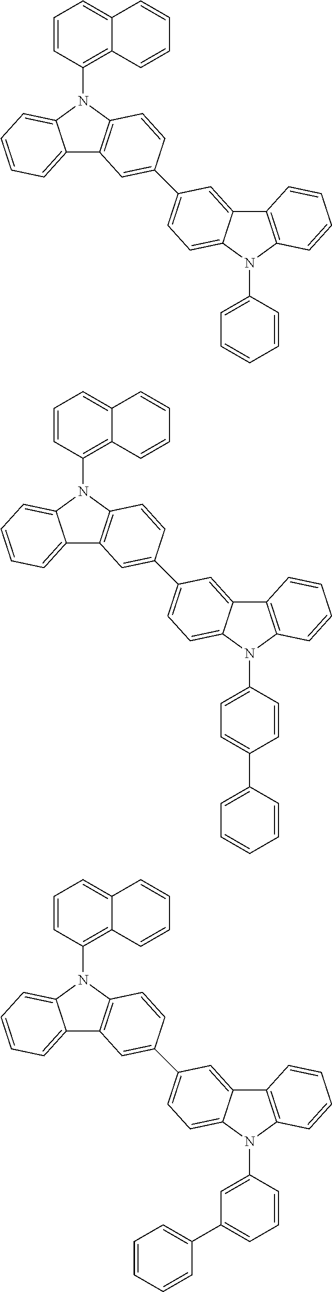 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2cccc(-n3c4ccccc4c4cc(-c5ccc6c(c5)c5ccccc5n6-c5cccc6ccccc56)ccc43)c2)cc1]

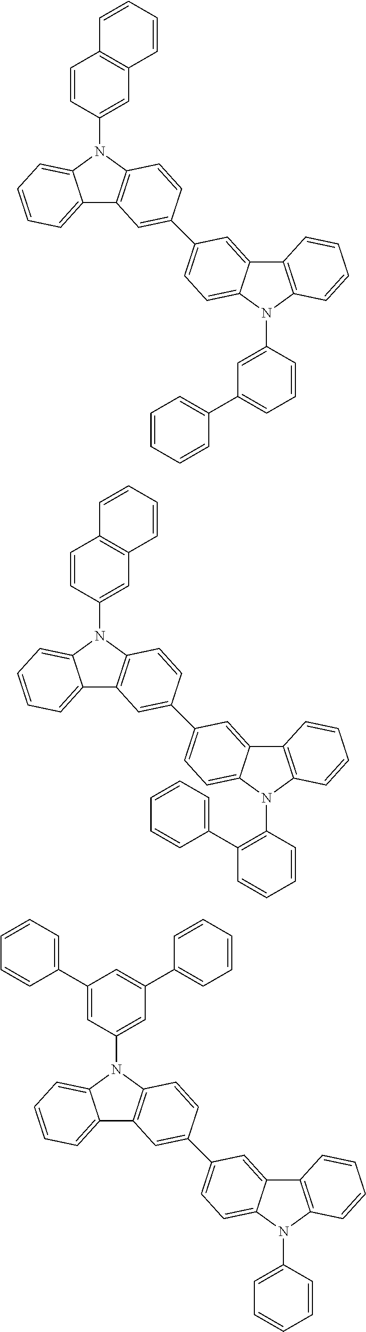

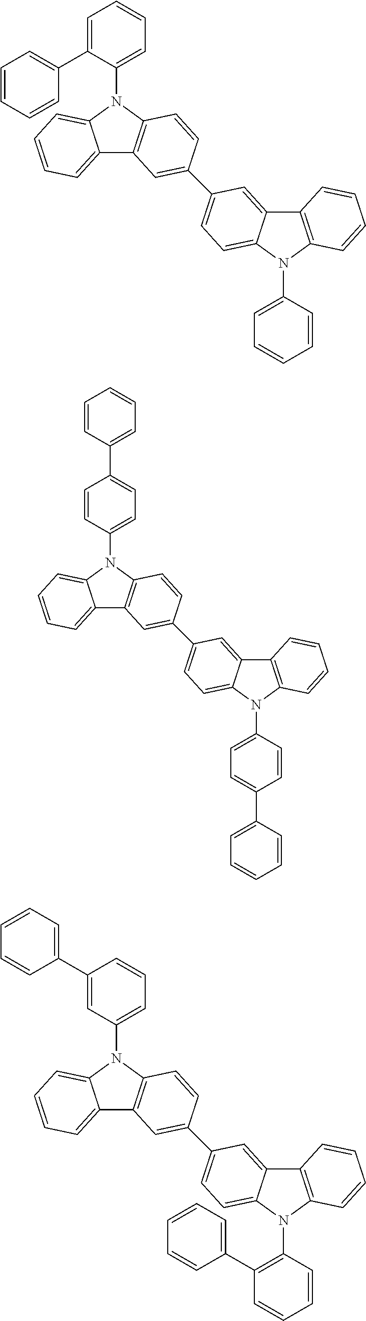 [SMILES: c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1]

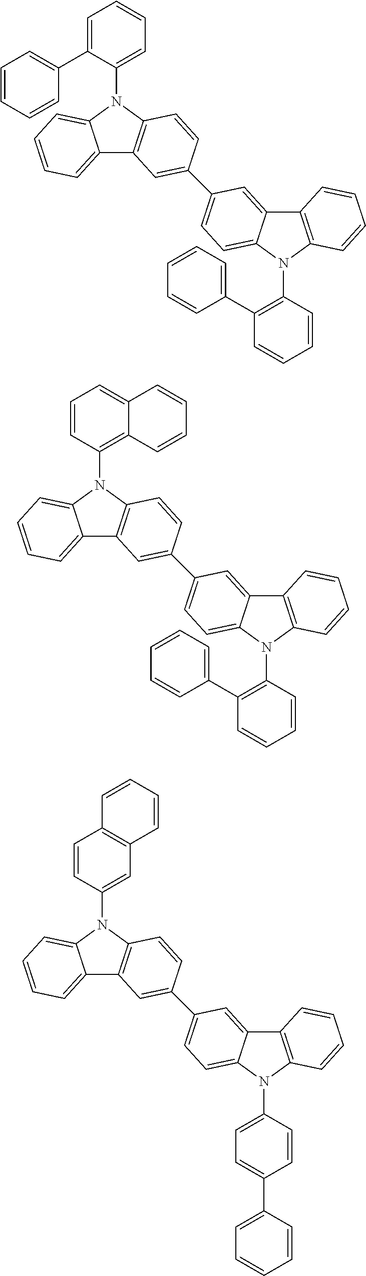 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccccc2-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1]

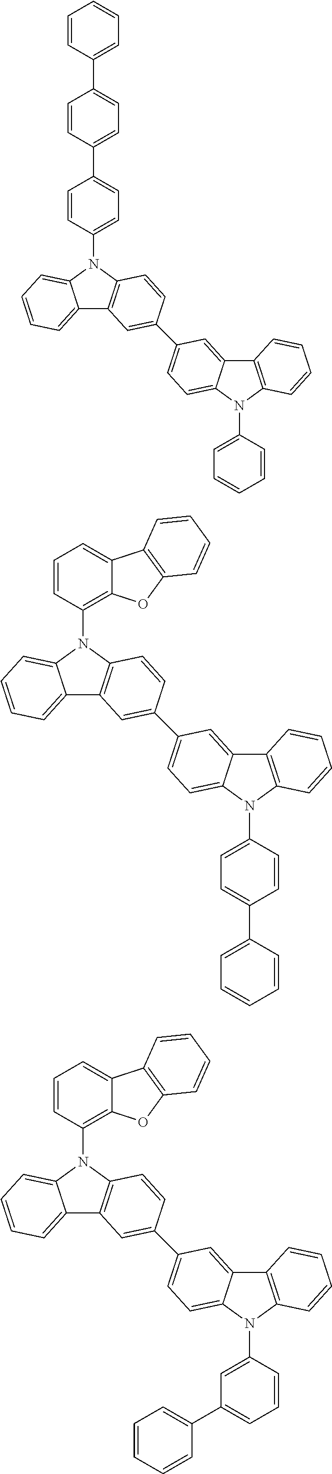

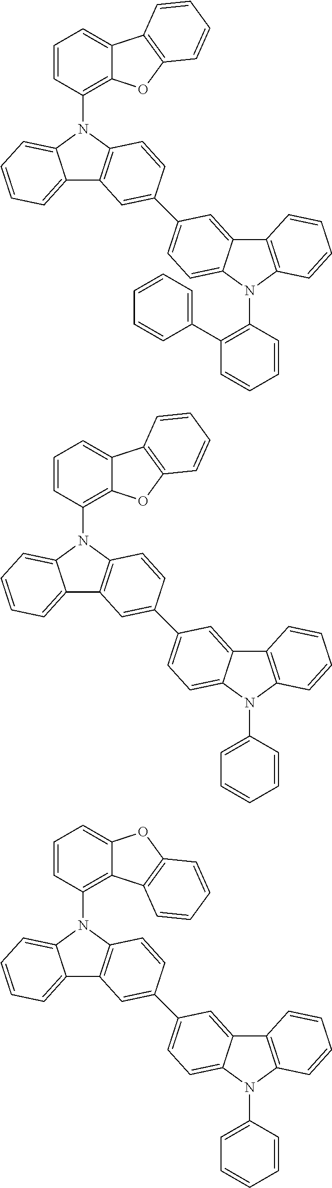 [SMILES: c1ccc(-c2ccccc2-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc5oc6ccccc6c45)ccc32)cc1]

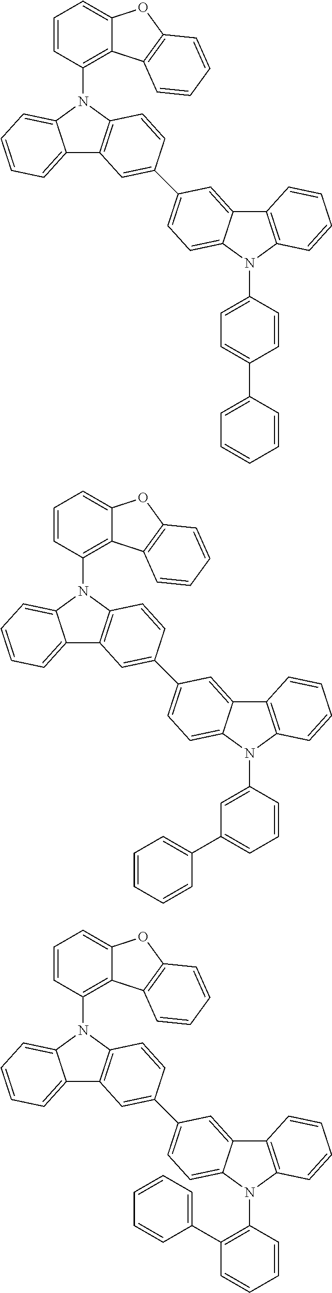

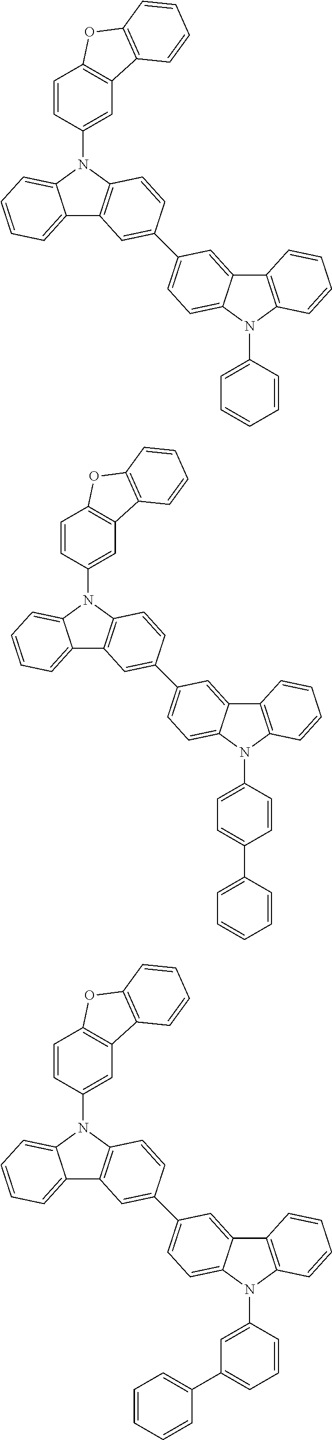 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

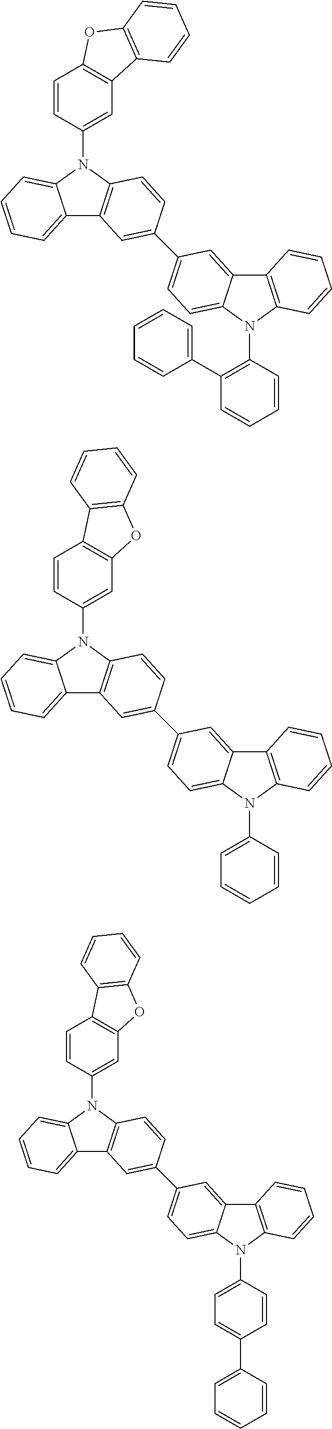 [SMILES: c1ccc(-c2ccccc2-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5c(c4)oc4ccccc45)ccc32)cc1 ; c1ccc(-c2ccc(-n3c4ccccc4c4cc(-c5ccc6c(c5)c5ccccc5n6-c5ccc6c(c5)oc5ccccc56)ccc43)cc2)cc1]

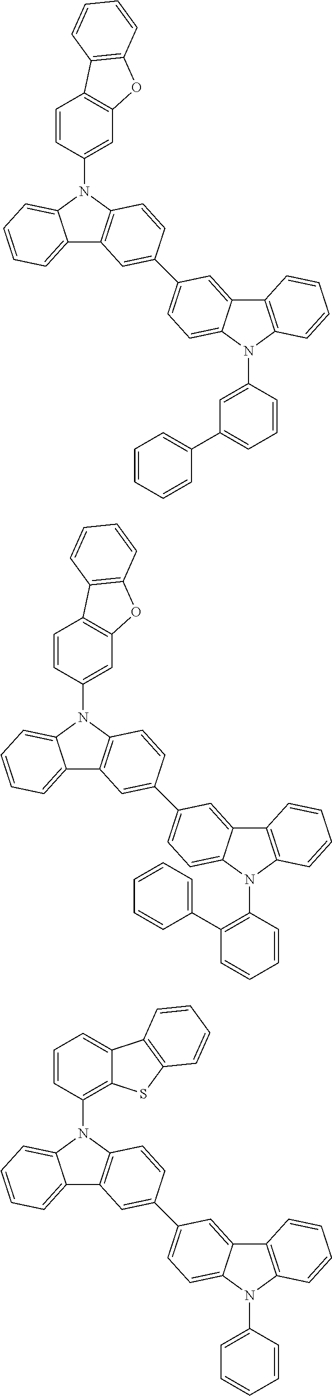

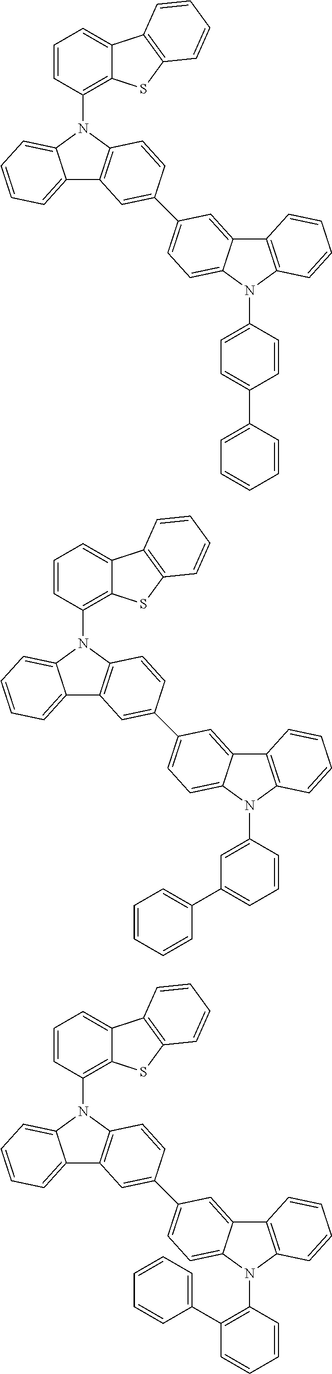

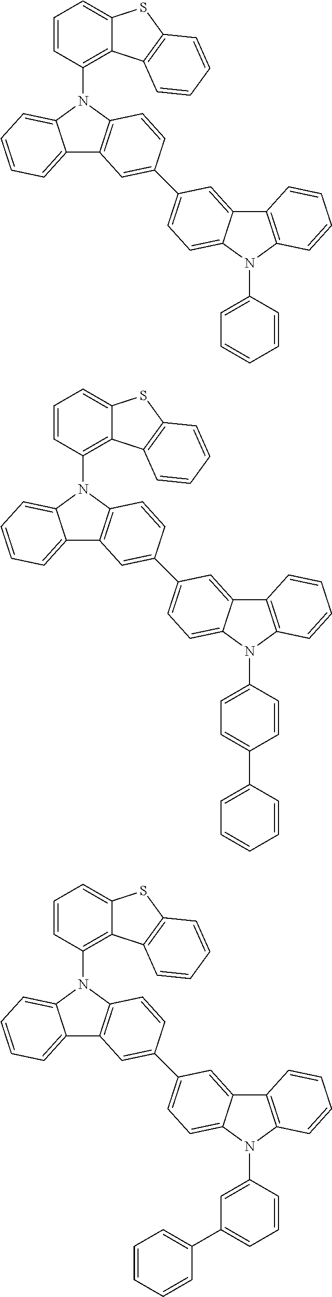 [SMILES: c1ccc(-n2c3ccccc3c3cc(-c4ccc5c(c4)c4ccccc4n5-c4cccc5sc6ccccc6c45)ccc32)cc1 ; c1ccc(-c2ccc(-n3c4ccccc4c4cc(-c5ccc6c(c5)c5ccccc5n6-c5cccc6sc7ccccc7c56)ccc43)cc2)cc1 ; c1ccc(-c2cccc(-n3c4ccccc4c4cc(-c5ccc6c(c5)c5ccccc5n6-c5cccc6sc7ccccc7c56)ccc43)c2)cc1]

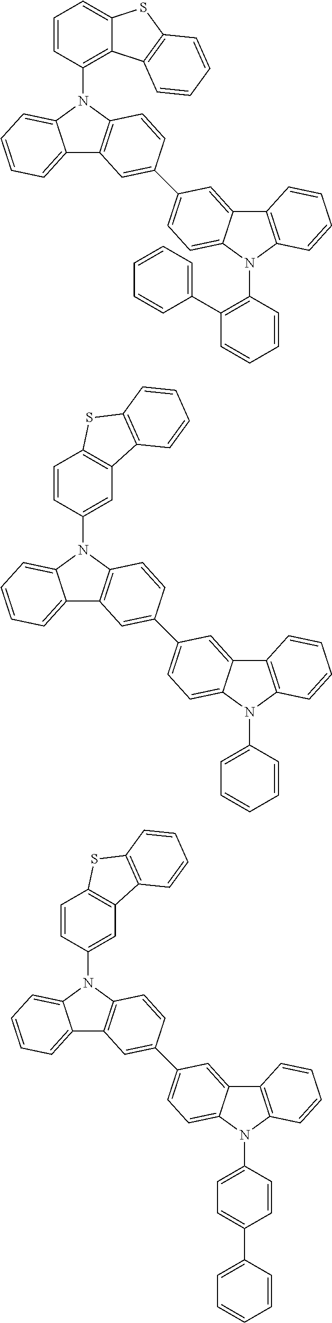 [SMILES: c1ccc(-c2ccccc2-n2c3ccccc3c3cc(-c4ccc5c(c4)c4ccccc4n5-c4cccc5sc6ccccc6c45)ccc32)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1]

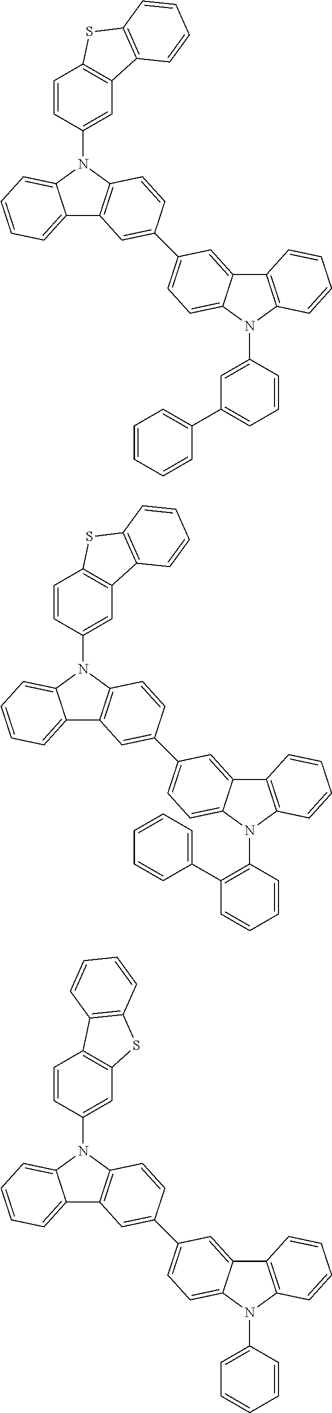

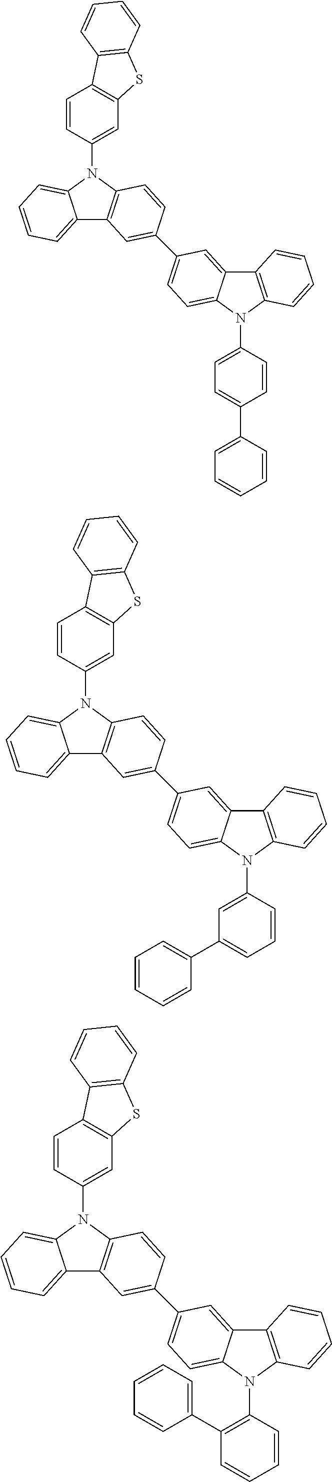

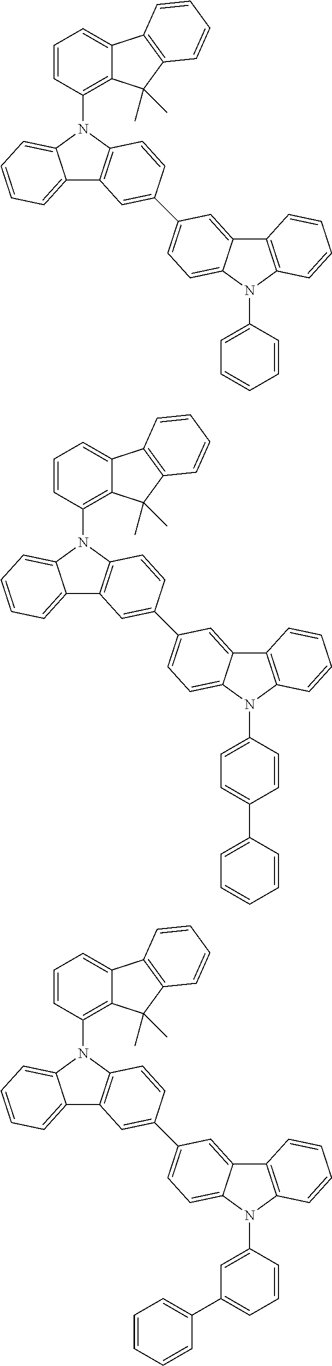 [SMILES: CC1(C)c2ccccc2-c2cccc(-n3c4ccccc4c4cc(-c5ccc6c(c5)c5ccccc5n6-c5ccccc5)ccc43)c21 ; CC1(C)c2ccccc2-c2cccc(-n3c4ccccc4c4cc(-c5ccc6c(c5)c5ccccc5n6-c5ccc(-c6ccccc6)cc5)ccc43)c21 ; CC1(C)c2ccccc2-c2cccc(-n3c4ccccc4c4cc(-c5ccc6c(c5)c5ccccc5n6-c5cccc(-c6ccccc6)c5)ccc43)c21]

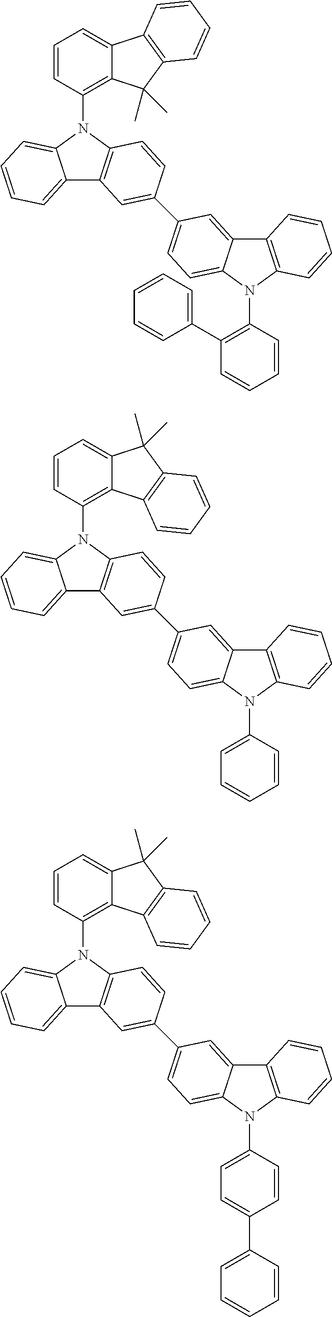 [SMILES: CC1(C)c2ccccc2-c2cccc(-n3c4ccccc4c4cc(-c5ccc6c(c5)c5ccccc5n6-c5ccccc5-c5ccccc5)ccc43)c21 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2c(-n3c4ccccc4c4cc(-c5ccc6c(c5)c5ccccc5n6-c5ccc(-c6ccccc6)cc5)ccc43)cccc21]

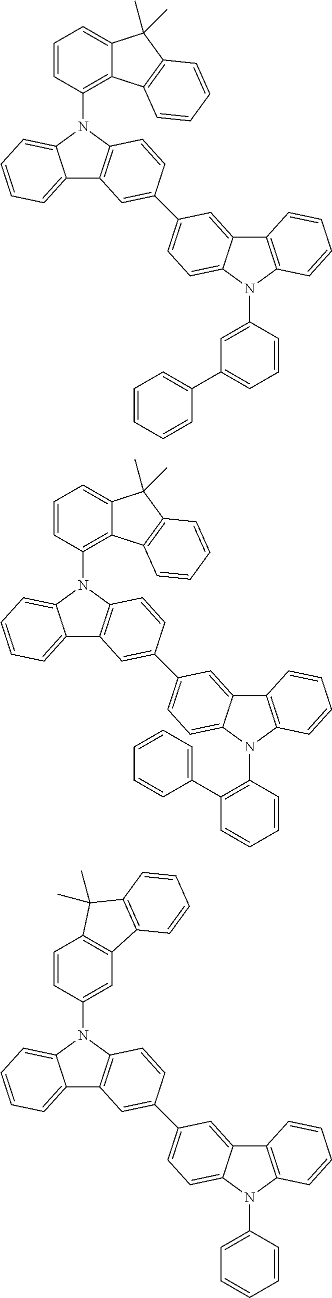

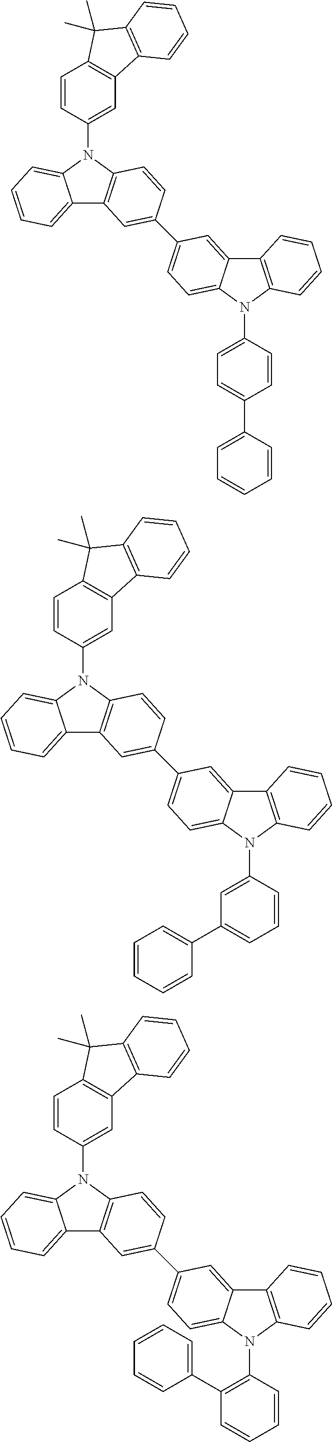

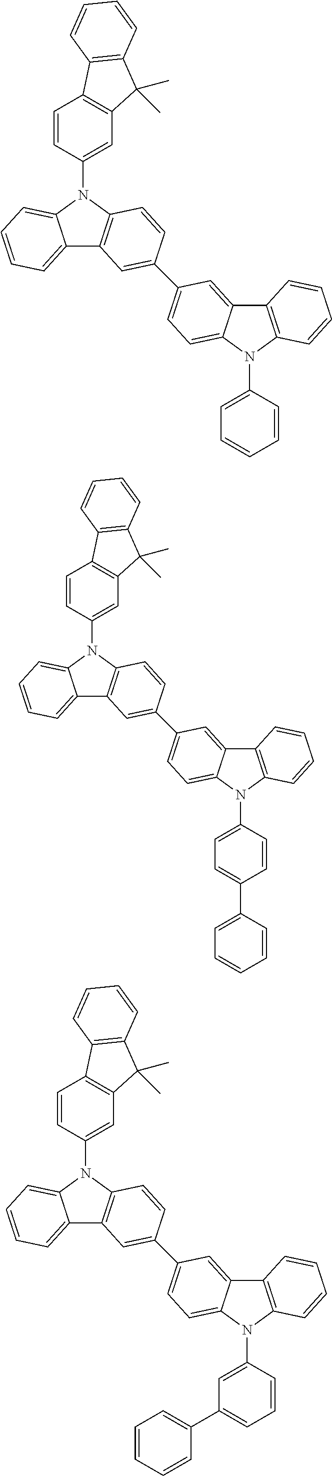 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cccc(-c6ccccc6)c5)ccc43)cc21]

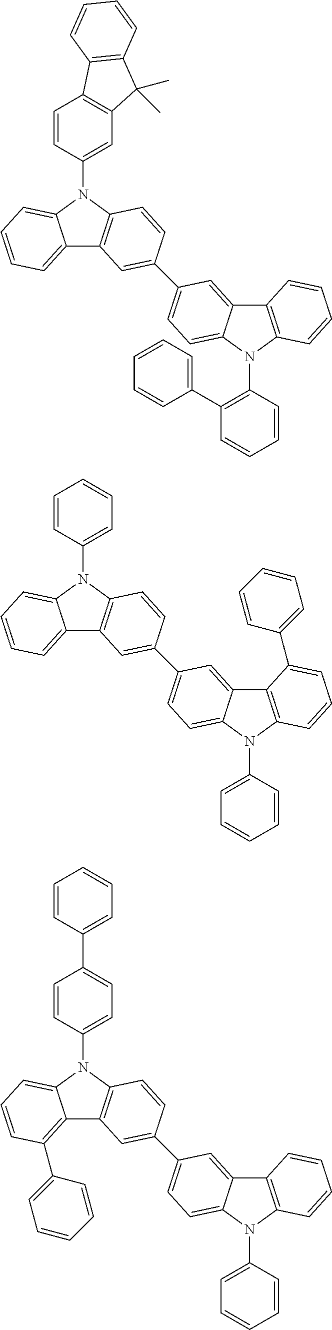 [SMILES: CC1(C)c2ccccc2-c2ccc(-n3c4ccccc4c4cc(-c5ccc6c(c5)c5ccccc5n6-c5ccccc5-c5ccccc5)ccc43)cc21 ; c1ccc(-c2cccc3c2c2cc(-c4ccc5c(c4)c4ccccc4n5-c4ccccc4)ccc2n3-c2ccccc2)cc1 ; c1ccc(-c2ccc(-n3c4ccc(-c5ccc6c(c5)c5ccccc5n6-c5ccccc5)cc4c4c(-c5ccccc5)cccc43)cc2)cc1]

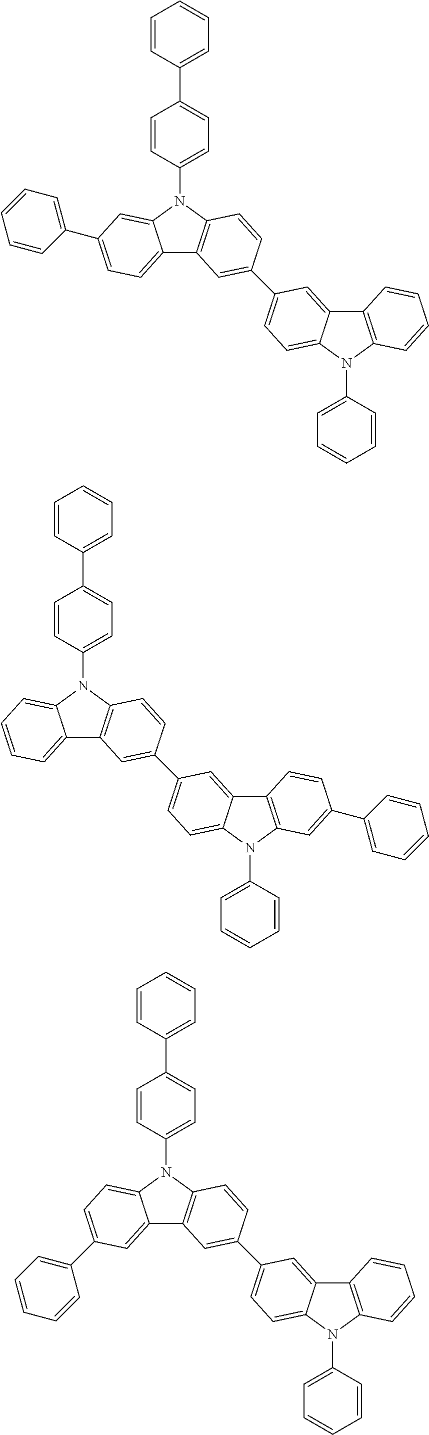 [SMILES: c1ccc(-c2ccc(-n3c4ccc(-c5ccc6c(c5)c5ccccc5n6-c5ccccc5)cc4c4ccc(-c5ccccc5)cc43)cc2)cc1 ; c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1]

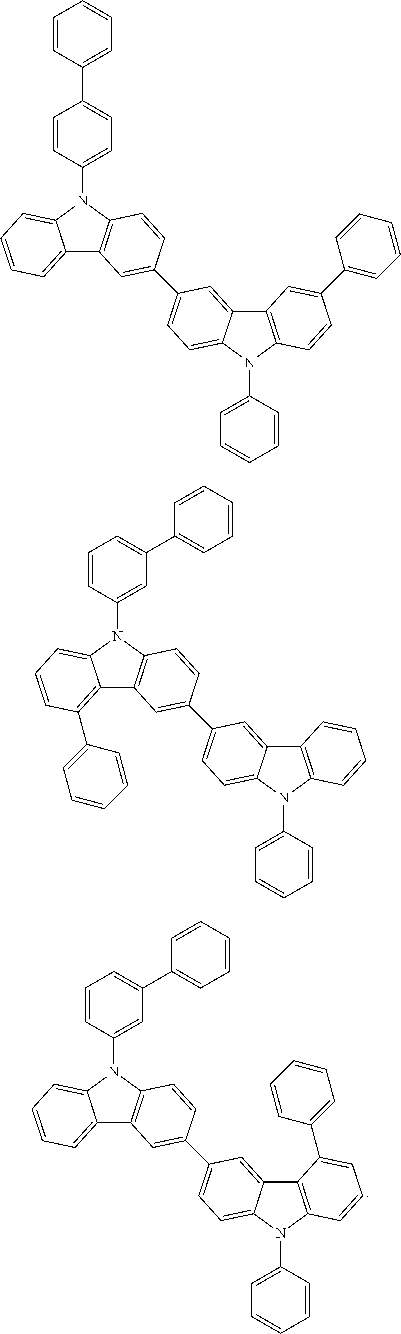

The compound of Chemical Formula 1 and the compound of Chemical Formula 2, which are two kinds of host materials, can be contained in the light emitting layer at a weight ratio of 10:90 to 90:10, for example, at a weight ratio of 50:50.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted with a substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

Further, the hole blocking layer refers to a layer which is formed on the light emitting layer, and preferably, is provided in contact with the light emitting layer, and thus severs to control electron mobility, to prevent excessive movement of holes, and to increase the probability of hole-electron bonding, thereby improving the efficiency of the organic light emitting device. The hole blocking layer includes a hole blocking material, and as an example of such hole blocking material, a compound into which an electron-withdrawing group is introduced, such as azine derivatives including triazine; triazole derivatives; oxadiazole derivatives; phenanthroline derivatives; phosphine oxide derivatives can be used, but is not limited thereto.

Further, the electron transport layer is a layer that receives the electrons from the electron injection layer and transports the electrons to the light emitting layer, and is formed on the light emitting layer and the hole blocking layer. The electron transport layer includes an electron transport material, and such an electron transport material is suitably a material which has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. Alternatively, fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like can be used together as the electron transport material, but are not limited thereto.

The electron injection layer is a layer that inject electrons from an electrode and is formed on the electron transport layer. Examples of the electron injection material included in the electron injection layer include LiF, NaCl, CsF, Li₂O, BaO, fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Here, the metal complex compound includes 8-hydroxy-quinolinato lithium, bis(8-hydroxyquinolinato)zinc, bis(8-hydroxy-quinolinato)copper, bis(8-hydroxyquinolinato)manganese, tris(8-hydroxyquinolinato)aluminum, tris(2-methyl-8-hydroxyquinolinato)-aluminum, tris(8-hydroxyquinolinato)gallium, bis(10-hydroxybenzo[h]-quinolinato)beryllium, bis(10-hydroxybenzo[h]quinolinato)zinc, bis(2-methyl-8-quinolinato) chlorogallium, bis(2-methyl-8-quinolinato) (o-cresolato)gallium, bis(2-methyl-8-quinolinato)(1-naphtholato)aluminum, bis(2-methyl-8-quinolinato)(2-naphtholato)gallium, and the like, but is not limited thereto.

The organic light emitting device according to the present disclosure can be a bottom emission type device, a top emission type device, or a double side emission type device, and in particular, it can be a bottom emission type light emitting device that requires relatively high luminous efficiency.

In addition, the compound of Chemical Formula 1 can be included in an organic solar cell or an organic transistor in addition to an organic light emitting device.

### EXAMPLES

The preparation of the compound of Chemical Formula 1 and the organic light emitting device including the same will be described in detail in the following examples. However, these examples are presented for illustrative purposes only, and are not intended to limit the scope of the present disclosure.

### Synthesis Example A: Preparation of Intermediate Compound Sub 1-1

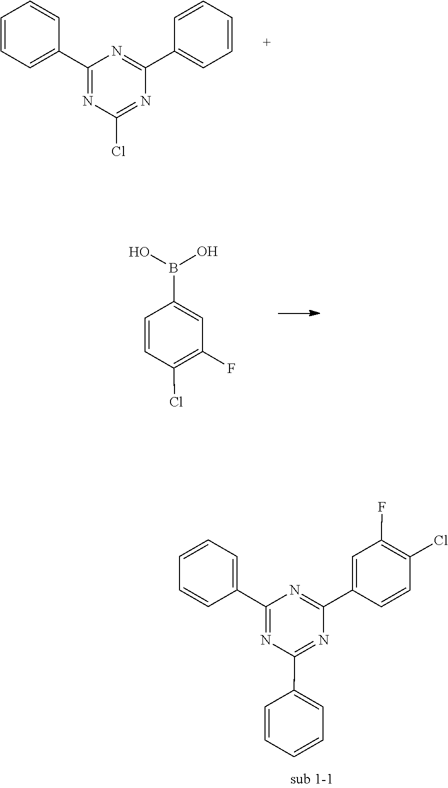 [SMILES: Clc1nc(-c2ccccc2)nc(-c2ccccc2)n1 ; OB(O)c1ccc(Cl)c(F)c1 ; Fc1cc(-c2nc(-c3ccccc3)nc(-c3ccccc3)n2)ccc1Cl]

2-Chloro-4,6-diphenyl-1,3,5-triazine (100 g, 374.5 mmol) and (4-chloro-3-fluorophenyl) boronic acid (136.8 g, 786.4 mmol) were added to 3000 mL of tetrahydrofuran under a nitrogen atmosphere, and the mixture was stirred and refluxed. Then, potassium carbonate (155.3 g, 1123.4 mmol) was dissolved in 155 ml of water and added thereto, and the mixture was sufficiently stirred and then tetrakistriphenyl-phosphinopalladium (17.3 g, 15 mmol) was added. After reacting for 2 hours, the reaction mixture was cooled to room temperature, then the organic layer and the aqueous layer were separated, and the organic layer was distilled. This was again dissolved in 6761 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a white solid compound sub 1-1 (79.8 g, 59%, MS: [M+H]⁺=362.1).

### Synthesis Example B: Preparation of Intermediate Compound Sub 2-1

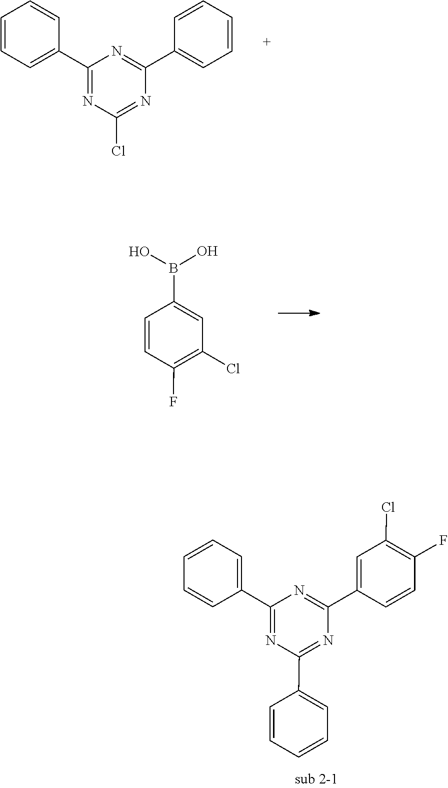 [SMILES: Clc1nc(-c2ccccc2)nc(-c2ccccc2)n1 ; OB(O)c1ccc(F)c(Cl)c1 ; Fc1ccc(-c2nc(-c3ccccc3)nc(-c3ccccc3)n2)cc1Cl]

2-Chloro-4,6-diphenyl-1,3,5-triazine (100 g, 374.5 mmol) and (2-chloro-4-fluorophenyl) boronic acid (136.8 g, 786.4 mmol) were added to 3000 mL of tetrahydrofuran under a nitrogen atmosphere, and the mixture was stirred and refluxed. Then, potassium carbonate (155.3 g, 1123.4 mmol) was dissolved in 155 ml of water and added thereto, and the mixture was sufficiently stirred and then tetrakistriphenyl-phosphinopalladium (17.3 g, 15 mmol) was added. After reacting for 3 hours, the reaction mixture was cooled to room temperature, then the organic layer and the aqueous layer were separated, and the organic layer was distilled. This was again dissolved in 6761 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a white solid compound sub 2-1 (79.8 g, 59%, MS: [M+H]⁺=362.1).

### Synthesis Example C: Preparation of Intermediate Compound Sub 3-1

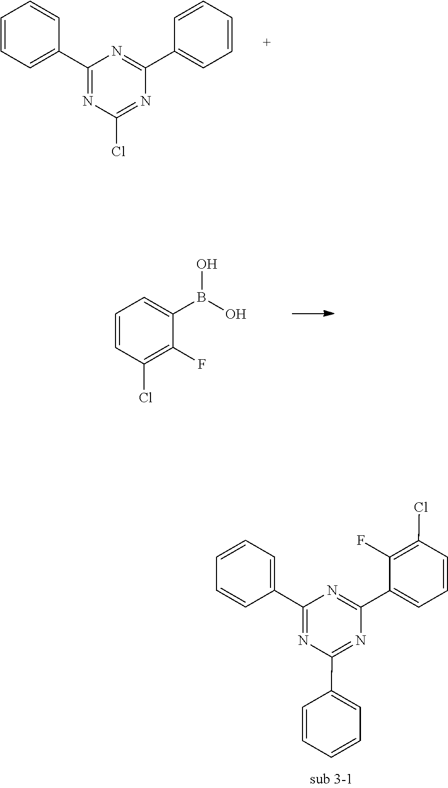 [SMILES: Clc1nc(-c2ccccc2)nc(-c2ccccc2)n1 ; OB(O)c1cccc(Cl)c1F ; Fc1c(Cl)cccc1-c1nc(-c2ccccc2)nc(-c2ccccc2)n1]

2-Chloro-4,6-diphenyl-1,3,5-triazine (100 g, 374.5 mmol) and (3-chloro-2-fluorophenyl) boronic acid (136.8 g, 786.4 mmol) were added to 3000 mL of tetrahydrofuran under a nitrogen atmosphere, and the mixture was stirred and refluxed. Then, potassium carbonate (155.3 g, 1123.4 mmol) was dissolved in 155 ml of water and added thereto, and the mixture was sufficiently stirred and then tetrakistriphenyl-phosphinopalladium (17.3 g, 15 mmol) was added. After reacting for 1 hours, the reaction mixture was cooled to room temperature, then the organic layer and the aqueous layer were separated, and the organic layer was distilled. This was again dissolved in 6761 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a white solid compound sub 3-1 (73 g, 54%, MS: [M+H]⁺=362.1).

### Synthesis Example D: Preparation of Intermediate Compound Sub 4-3

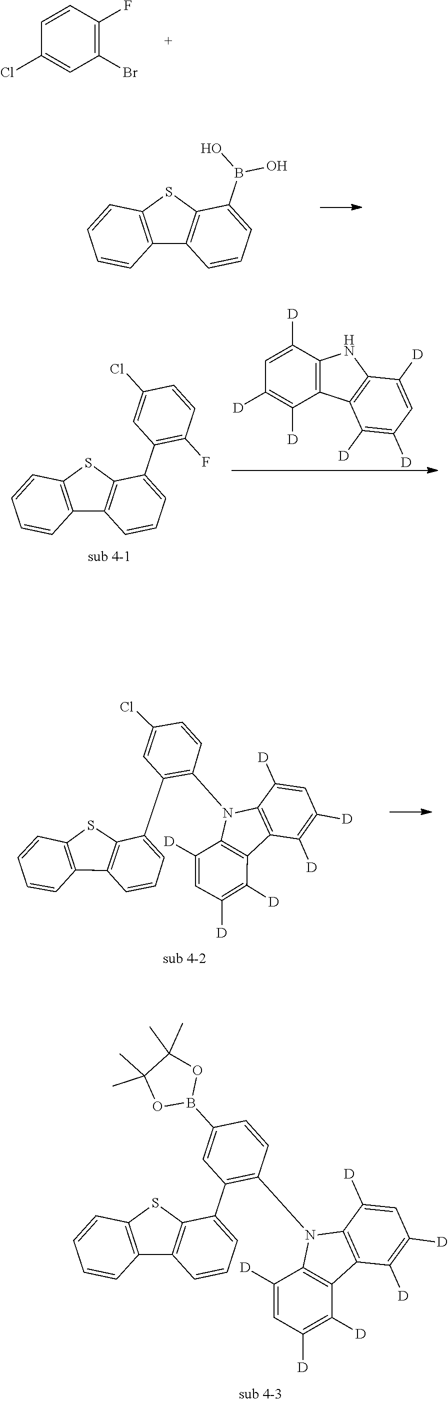

2-Bromo-4-chloro-1-fluorobenzene (100 g, 481 mmol) and dibenzo[b, d]thiophen-4-ylboronic acid (109.7 g, 481 mmol) were added to 2000 mL of tetrahydrofuran under a nitrogen atmosphere, and the mixture was stirred and refluxed. Then, potassium carbonate (199.4 g, 1442.9 mmol) was dissolved in 199 ml of water and added thereto, and the mixture was sufficiently stirred and then tetrakistriphenyl-phosphinopalladium (16.7 g, 14.4 mmol) was added. After reacting for 1 hours, the reaction mixture was cooled to room temperature, then the organic layer and the aqueous layer were separated, and the organic layer was distilled. The solid was dissolved in 7504 mL of chloroform, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a white solid compound sub 4-1 (87 g, 58%, MS: [M+H]⁺=313).

Next, sub 4-1 (50 g, 160.2 mmol) and 9H-carbazole-1,3,4,5,6,8-d6 (27.7 g, 160.2 mmol) were added to 1000 ml of xylene under a nitrogen atmosphere, and the mixture was stirred and refluxed. Then, sodium tert-butoxide (46.2 g, 480.7 mmol) was added thereto, and sufficiently stirred and then bis(tri-tert-butylphosphine) palladium (2.5 g, 4.8 mmol) was added. After reacting for 5 hours, the reaction mixture was cooled to room temperature, and the resulting solid was filtered. The solid was added to and dissolved in 2236 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid compound sub 4-2 (58.9 g, 79%, MS: [M+H]⁺=466.1).

Next, sub-4-2 (50 g, 107.5 mmol) and bis(pinacolato)diboron (27.6 g, 118.2 mmol) were added to 1000 ml of 1,4-dioxane (Diox) under a nitrogen atmosphere, and the mixture was stirred and refluxed. Then, potassium acetate (31 g, 322.5 mmol) was added thereto and sufficiently stirred, and then palladium dibenzylidene acetone palladium (1.9 g, 3.2 mmol) and tricyclohexylphosphine (1.8 g, 6.4 mmol) were added. After reacting for 6 hours, the reaction mixture was cooled to room temperature and then the organic layer was subjected to filtration treatment to remove a salt, and then the filtered organic layer was distilled. This was added again to 599 ml of chloroform, dissolved and washed twice with water. The organic layer was then separated, anhydrous magnesium sulfate was added, stirred and then filtered. The filtrate was distilled under reduced pressure. The concentrated compound was recrystallized from chloroform and ethanol to prepare a gray solid compound sub 4-3 (53.3 g, 89%, MS: [M+H]⁺=558.3).

### Synthesis Example 1: Preparation of Compound 1

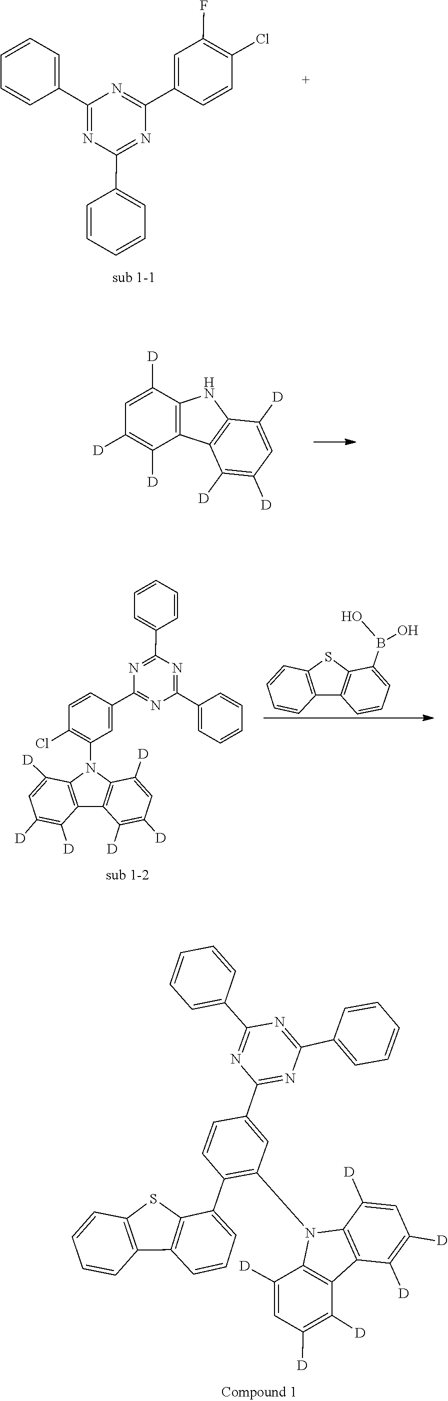

sub 1-1 (50 g, 138.5 mmol) and 9H-carbazole-1,3,4,5,6,8-d6 (24 g, 138.5 mmol) were added to 1000 ml of xylene under a nitrogen atmosphere, and the mixture was stirred and refluxed. Then, sodium tert-butoxide (39.9 g, 415.4 mmol) was added thereto, and sufficiently stirred and then bis(tri-tert-butylphosphine) palladium (2.1 g, 4.2 mmol) was added. After reacting for 3 hours, the reaction mixture was cooled to room temperature, and the resulting solid was filtered. The solid was added to and dissolved in 2136 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid compound sub 1-2 (47.7 g, 67%, MS: [M+H]⁺=515.2).

Next, sub 1-2 (20 g, 38.9 mmol) and dibenzo[b,d]thiophen-4-ylboronic acid (8.9 g, 38.9 mmol) were added to 400 mL of Diox under a nitrogen atmosphere, and the mixture was stirred and refluxed. Then, potassium triphosphate (24.8 g, 116.7 mmol) was dissolved in 25 ml of water and added thereto, and the mixture was sufficiently stirred and then dibenzylideneacetone palladium (0.7 g, 1.2 mmol) and tricyclohexylphosphine (0.7 g, 2.3 mmol) were added. After reacting for 6 hours, the reaction mixture was cooled to room temperature, and then the resulting solid was filtered. The solid was added to 773 mL of chloroform, dissolved, washed twice with water. The organic layer was then separated, anhydrous magnesium sulfate was added, stirred and then filtered. The filtrate was distilled under reduced pressure. The concentrated compound was recrystallized from chloroform and ethyl acetate to prepare a yellow solid Compound 1 (3.9 g, 15%, MS: [M+H]⁺=663.2).

### Synthesis Example 2: Preparation of Compound 2

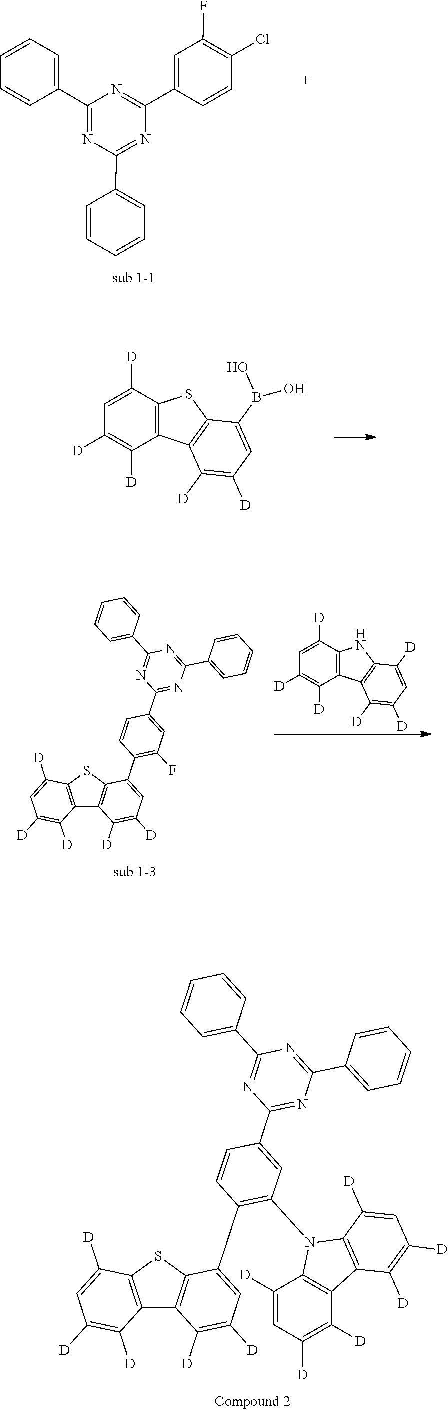

sub 1-1 (30 g, 83.1 mmol) and (dibenzo[b,d]thiophen-4-yl-1,2,6,8,9-d5) boronic acid (40.7 g, 174.5 mmol) were added to 900 mL of tetrahydrofuran under a nitrogen atmosphere, and the mixture was stirred and refluxed. Then, potassium carbonate (34.4 g, 249.3 mmol) was dissolved in 34 ml of water and added thereto, and the mixture was sufficiently stirred and then tetrakistriphenyl-phosphinopalladium (3.8 g, 3.3 mmol) was added. After reacting for 1 hours, the reaction mixture was cooled to room temperature, and the resulting solid was filtered. The solid was added to and dissolved in 2136 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid compound sub 1-3 (25.2 g, 59%, MS: [M+H]⁺=515.2).

Next, sub 1-3 (20 g, 38.9 mmol) and 9H-carbazole-1,3,4,5,6,8-d6 (6.7 g, 38.9 mmol) were added to 400 ml of xylene under a nitrogen atmosphere, and the mixture was stirred and refluxed. Then, sodium tert-butoxide (11.2 g, 116.7 mmol) was added thereto, and sufficiently stirred and then bis(tri-tert-butylphosphine) palladium (0.6 g, 1.2 mmol) was added. After reacting for 1 hours, the reaction mixture was cooled to room temperature, and the resulting solid was filtered. The solid was added to and dissolved in 779 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 2 (17.9 g, 69%, MS: [M+H]⁺=668.3).

### Synthesis Example 3: Preparation of Compound 3

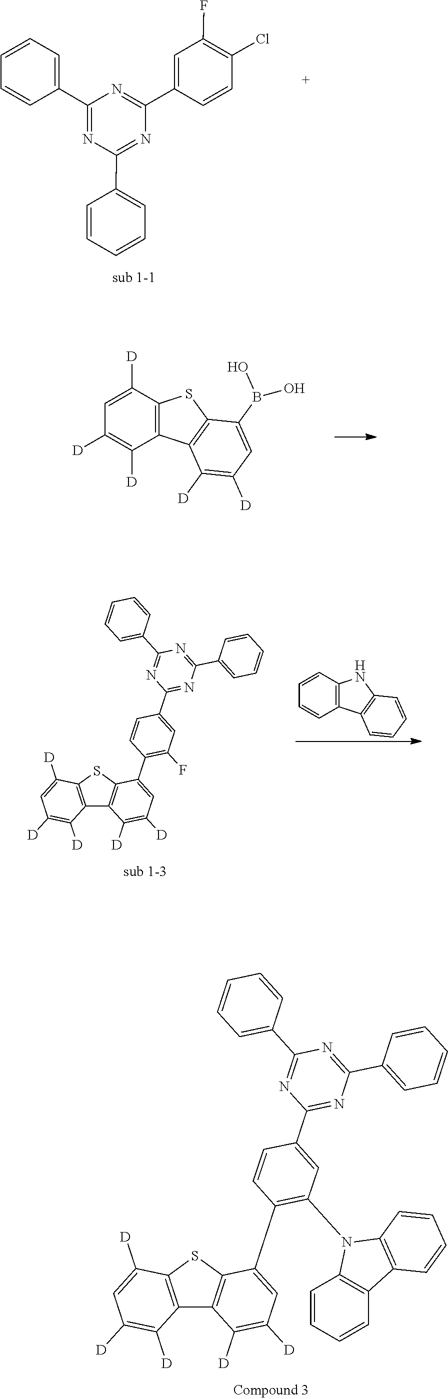

A solid Compound sub 1-3 was prepared in the same manner as in Synthesis Example 2.

Next, sub 1-3 (20 g, 38.9 mmol) and 9H-carbazole (6.5 g, 38.9 mmol) were added to 400 mL of xylene under a nitrogen atmosphere, and the mixture was stirred and refluxed. Then, sodium tert-butoxide (11.2 g, 116.7 mmol) was added thereto, and sufficiently stirred and then bis(tri-tert-butylphosphine) palladium (0.6 g, 1.2 mmol) was added. After reacting for 2 hours, the reaction mixture was cooled to room temperature, and the resulting solid was filtered. The solid was added to and dissolved in 772 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 3 (14.1 g, 55%, MS: [M+H]⁺=662.2).

### Synthesis Example 4: Preparation of Compound 4

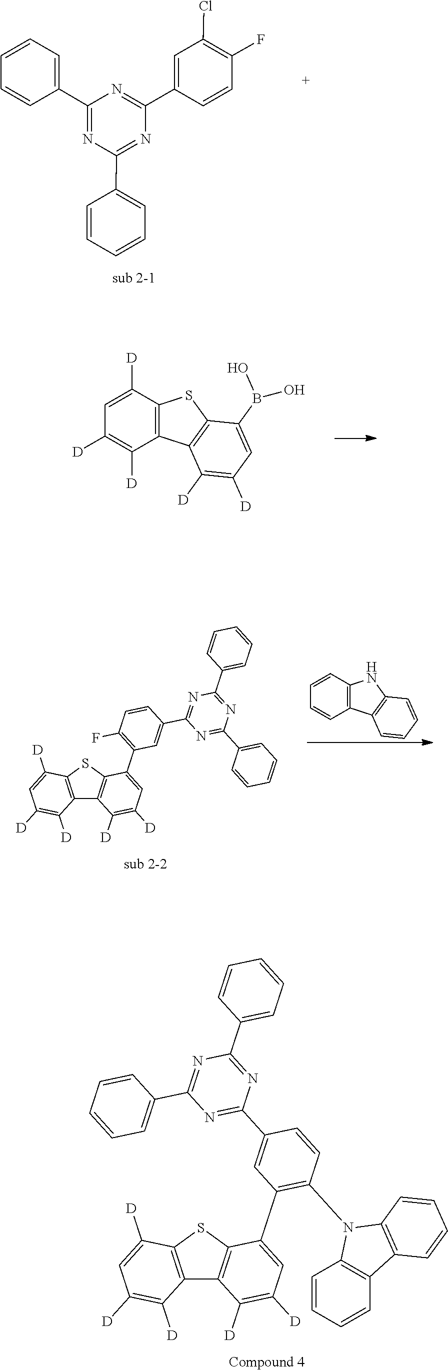 [SMILES: Fc1ccc(-c2nc(-c3ccccc3)nc(-c3ccccc3)n2)cc1Cl ; [2H]c1cc([2H])c2sc3c(B(O)O)cc([2H])c([2H])c3c2c1[2H] ; [2H]c1cc([2H])c2sc3c(-c4cc(-c5nc(-c6ccccc6)nc(-c6ccccc6)n5)ccc4F)cc([2H])c([2H])c3c2c1[2H] ; c1ccc2c(c1)[nH]c1ccccc12 ; [2H]c1cc([2H])c2sc3c(-c4cc(-c5nc(-c6ccccc6)nc(-c6ccccc6)n5)ccc4-n4c5ccccc5c5ccccc54)cc([2H])c([2H])c3c2c1[2H]]

sub 2-1 (30 g, 83.1 mmol) and (dibenzo[b,d]thiophen-4-yl-1,2,6,8,9-d5) boronic acid (40.7 g, 174.5 mmol) were added to 900 mL of tetrahydrofuran under a nitrogen atmosphere, and the mixture was stirred and refluxed. Then, potassium carbonate (34.4 g, 249.3 mmol) was dissolved in 34 ml of water and added thereto, and the mixture was sufficiently stirred and then tetrakistriphenyl-phosphinopalladium (3.8 g, 3.3 mmol) was added. After reacting for 1 hours, the reaction mixture was cooled to room temperature, and the resulting solid was filtered. The solid was added to and dissolved in 2136 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid compound sub 2-2 (27.3 g, 64%, MS: [M+H]⁺=515.2).

Next, sub 2-2 (20 g, 38.9 mmol) and 9H-carbazole (6.5 g, 38.9 mmol) were added to 400 mL of xylene under a nitrogen atmosphere, and the mixture was stirred and refluxed. Then, sodium tert-butoxide (11.2 g, 116.7 mmol) was added thereto, and sufficiently stirred and then bis(tri-tert-butylphosphine) palladium (0.6 g, 1.2 mmol) was added. After reacting for 5 hours, the reaction mixture was cooled to room temperature, and the resulting solid was filtered. The solid was added to and dissolved in 772 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 4 (19.3 g, 75%, MS: [M+H]⁺=662.2).

### Synthesis Example 5: Preparation of Compound 5

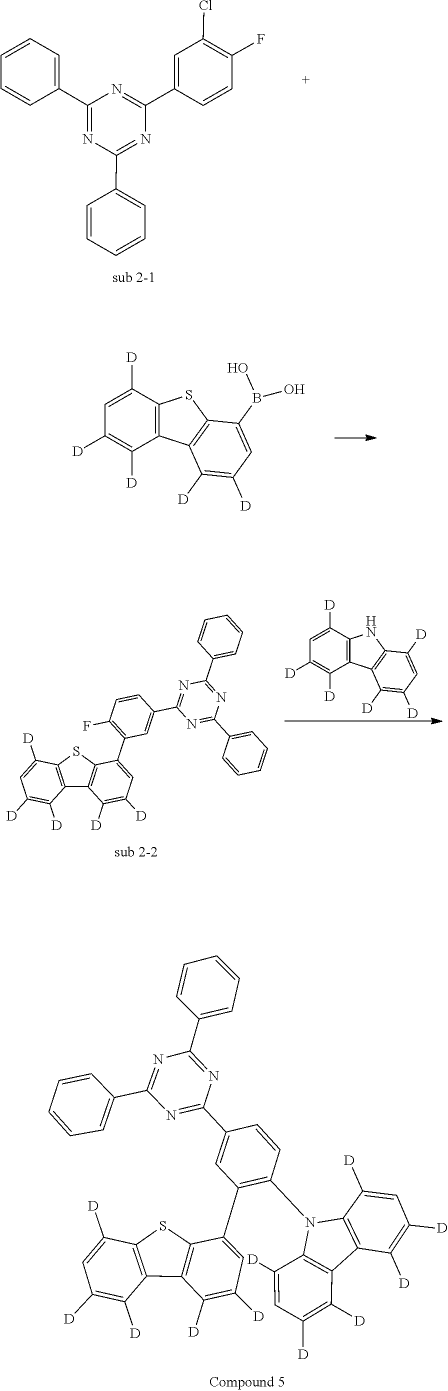 [SMILES: Fc1ccc(-c2nc(-c3ccccc3)nc(-c3ccccc3)n2)cc1Cl ; [2H]c1cc([2H])c2sc3c(B(O)O)cc([2H])c([2H])c3c2c1[2H] ; [2H]c1cc([2H])c2sc3c(-c4cc(-c5nc(-c6ccccc6)nc(-c6ccccc6)n5)ccc4F)cc([2H])c([2H])c3c2c1[2H] ; [2H]c1cc([2H])c2[nH]c3c([2H])cc([2H])c([2H])c3c2c1[2H] ; [2H]c1cc([2H])c2sc3c(-c4cc(-c5nc(-c6ccccc6)nc(-c6ccccc6)n5)ccc4-n4c5c([2H])cc([2H])c([2H])c5c5c([2H])c([2H])cc([2H])c54)cc([2H])c([2H])c3c2c1[2H]]

A solid Compound sub 2-2 was prepared in the same manner as in Synthesis Example 4.

Next, sub 2-2 (20 g, 38.9 mmol) and 9H-carbazole-1,3,4,5,6,8-d6 (6.7 g, 38.9 mmol) were added to 400 mL of xylene under a nitrogen atmosphere, and the mixture was stirred and refluxed. Then, sodium tert-butoxide (11.2 g, 116.7 mmol) was added thereto, and sufficiently stirred and then bis(tri-tert-butylphosphine) palladium (0.6 g, 1.2 mmol) was added. After reacting for 1 hours, the reaction mixture was cooled to room temperature, and the resulting solid was filtered. The solid was added to and dissolved in 779 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 5 (17.9 g, 69%, MS: [M+H]⁺=668.3).

### Synthesis Example 6: Preparation of Compound 6

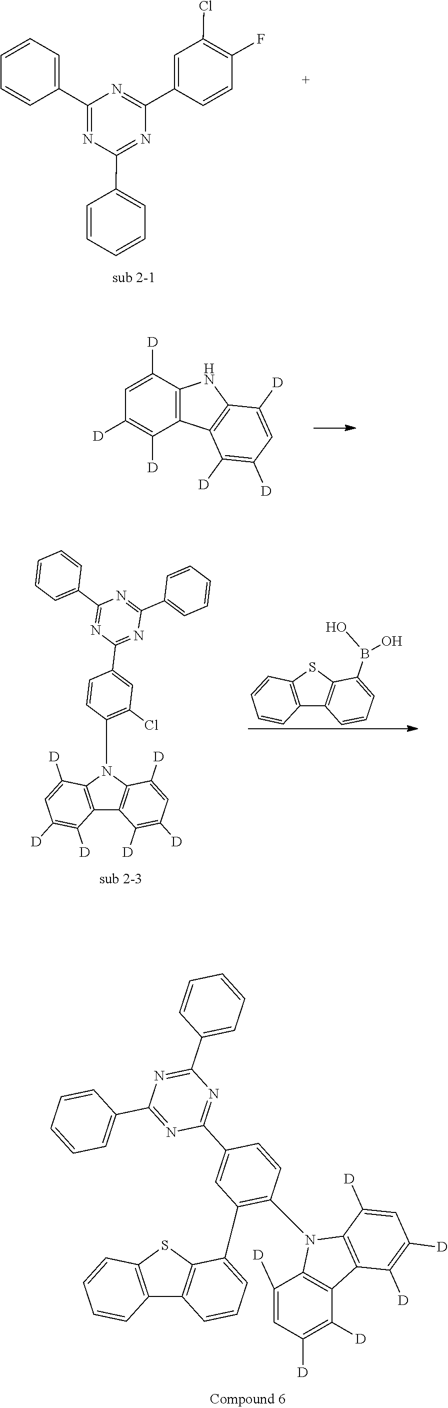 [SMILES: Fc1ccc(-c2nc(-c3ccccc3)nc(-c3ccccc3)n2)cc1Cl ; [2H]c1cc([2H])c2[nH]c3c([2H])cc([2H])c([2H])c3c2c1[2H] ; [2H]c1cc([2H])c2c(c1[2H])c1c([2H])c([2H])cc([2H])c1n2-c1ccc(-c2nc(-c3ccccc3)nc(-c3ccccc3)n2)cc1Cl ; OB(O)c1cccc2c1sc1ccccc12 ; [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1sc1ccccc12]

sub 2-1 (20 g, 55.4 mmol) and 9H-carbazole-1,3,4,5,6,8-d6 (9.6 g, 55.4 mmol) were added to 400 ml of xylene under a nitrogen atmosphere, and the mixture was stirred and refluxed. Then, sodium tert-butoxide (16 g, 166.2 mmol) was added thereto, and sufficiently stirred and then bis(tri-tert-butylphosphine) palladium (0.8 g, 1.7 mmol) was added. After reacting for 1 hours, the reaction mixture was cooled to room temperature, and the resulting solid was filtered. The solid 10 was added to and dissolved in 854 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound sub 2-3 (22.8 g, 80%, MS: [M+H]⁺=515.2).

sub 2-3 (30 g, 58.3 mmol) and dibenzo[b, d]thiophen-4-ylboronic acid (27.9 g, 122.5 mmol) were added to 900 ml of tetrahydrofuran under a nitrogen atmosphere, and the mixture was stirred and refluxed. Then, potassium carbonate (24.2 g, 175 mmol) was dissolved in 24 mL of water and added thereto, and the mixture was sufficiently stirred and then tetrakistriphenyl-phosphinopalladium (2.7 g, 2.3 mmol) was added. After reacting for 3 hours, the reaction mixture was cooled to room temperature, and the resulting solid was filtered. The solid was added to and dissolved in 1932 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 6 (22 g, 57%, MS: [M+H]⁺=663.2).

### Synthesis Example 7: Preparation of Compound 7

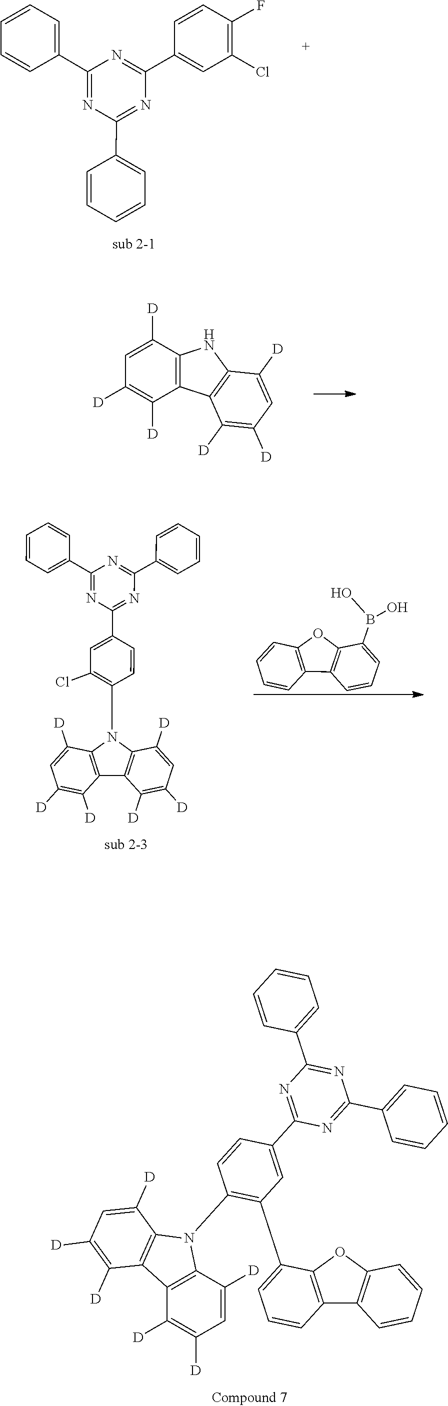 [SMILES: Fc1ccc(-c2nc(-c3ccccc3)nc(-c3ccccc3)n2)cc1Cl ; [2H]c1cc([2H])c2[nH]c3c([2H])cc([2H])c([2H])c3c2c1[2H] ; [2H]c1cc([2H])c2c(c1[2H])c1c([2H])c([2H])cc([2H])c1n2-c1ccc(-c2nc(-c3ccccc3)nc(-c3ccccc3)n2)cc1Cl ; OB(O)c1cccc2c1oc1ccccc12 ; [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1oc1ccccc12]

A solid compound sub 2-3 was prepared in the same manner as in Synthesis Example 6.

Next, sub 2-3 (30 g, 83.1 mmol) and dibenzo[b,d]furan-4-ylboronic acid (37 g, 174.5 mmol) were added to 900 ml of tetrahydrofuran under a nitrogen atmosphere, and the mixture was stirred and refluxed. Then, potassium carbonate (34.4 g, 249.3 mmol) was dissolved in 34 ml of water and added thereto, and the mixture was sufficiently stirred and then tetrakistriphenyl-phosphinopalladium (3.8 g, 3.3 mmol) was added. After reacting for 2 hours, the reaction mixture was cooled to room temperature, and the resulting solid was filtered. The solid was added to and dissolved in 2136 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 7 (26.1 g, 61%, MS: [M+H]⁺=515.2).

### Synthesis Example 8: Preparation of Compound 8

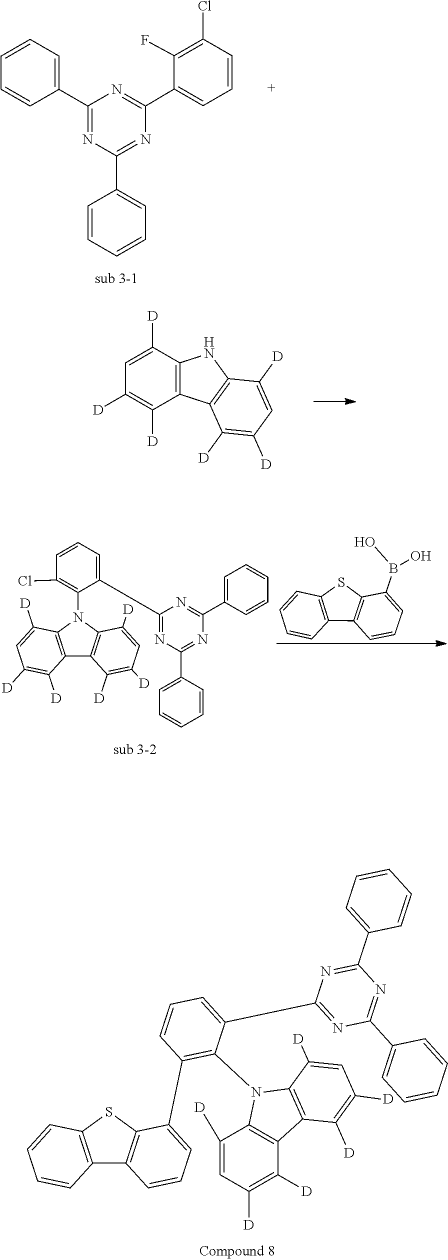

sub 3-1 (50 g, 138.5 mmol) and 9H-carbazole-1,3,4,5,6,8-d6 (24 g, 138.5 mmol) were added to 1000 ml of xylene under a nitrogen atmosphere, and the mixture was stirred and refluxed. Then, sodium tert-butoxide (39.9 g, 415.4 mmol) was added thereto, and sufficiently stirred and then bis(tri-tert-butylphosphine) palladium (2.1 g, 4.2 mmol) was added. After reacting for 1 hours, the reaction mixture was cooled to room temperature, and the resulting solid was filtered. The solid was added to and dissolved in 2136 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound sub 3-2 (44.1 g, 62%, MS: [M+H]⁺=515.2).

Next, sub 3-2 (30 g, 58.3 mmol) and dibenzo[b,d]thiophen-4-ylboronic acid (27.9 g, 122.5 mmol) were added to 900 ml of tetrahydrofuran under a nitrogen atmosphere, and the mixture was stirred and refluxed. Then, potassium carbonate (24.2 g, 175 mmol) was dissolved in 24 mL of water and added thereto, and the mixture was sufficiently stirred and then tetrakistriphenyl-phosphinopalladium (2.7 g, 2.3 mmol) was added. After reacting for 2 hours, the reaction mixture was cooled to room temperature, and the resulting solid was filtered. The solid was added to and dissolved in 1932 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 8 (29.4 g, 76%, MS: [M+H]⁺=663.2).

### Synthesis Example 9: Preparation of Compound 9

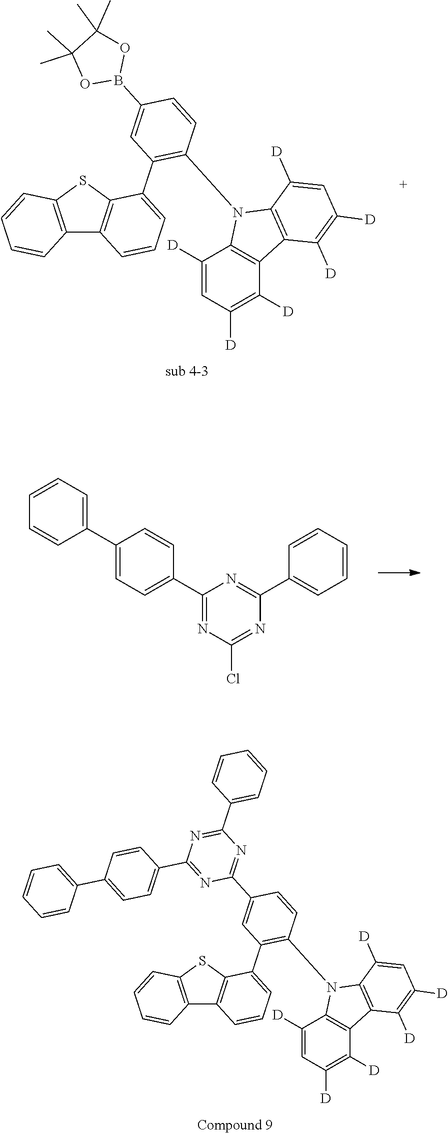 [SMILES: [2H]c1cc([2H])c2c(c1[2H])c1c([2H])c([2H])cc([2H])c1n2-c1ccc(B2OC(C)(C)C(C)(C)O2)cc1-c1cccc2c1sc1ccccc12 ; Clc1nc(-c2ccccc2)nc(-c2ccc(-c3ccccc3)cc2)n1 ; [2H]c1cc([2H])c2c(c1[2H])c1c([2H])c([2H])cc([2H])c1n2-c1ccc(-c2nc(-c3ccccc3)nc(-c3ccc(-c4ccccc4)cc3)n2)cc1-c1cccc2c1sc1ccccc12]

sub 4-3 (20 g, 35.9 mmol) and 2-([1,1′-biphenyl]-4-yl)-4-chloro-6-phenyl-1,3,5-triazine (12.3 g, 35.9 mmol) were added to 600 ml of tetrahydrofuran under a nitrogen atmosphere, and the mixture was stirred and refluxed. Then, potassium carbonate (14.9 g, 107.7 mmol) was dissolved in 15 ml of water and added thereto, and the mixture was sufficiently stirred and then tetrakistriphenyl-phosphinopalladium (1.2 g, 1.1 mmol) was added. After reacting for 2 hours, the reaction mixture was cooled to room temperature, and the resulting solid was filtered. The solid was added to and dissolved in 1325 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 9 (17.5 g, 66%, MS: [M+H]⁺=739.3).

### Synthesis Example 10: Preparation of Compound 10

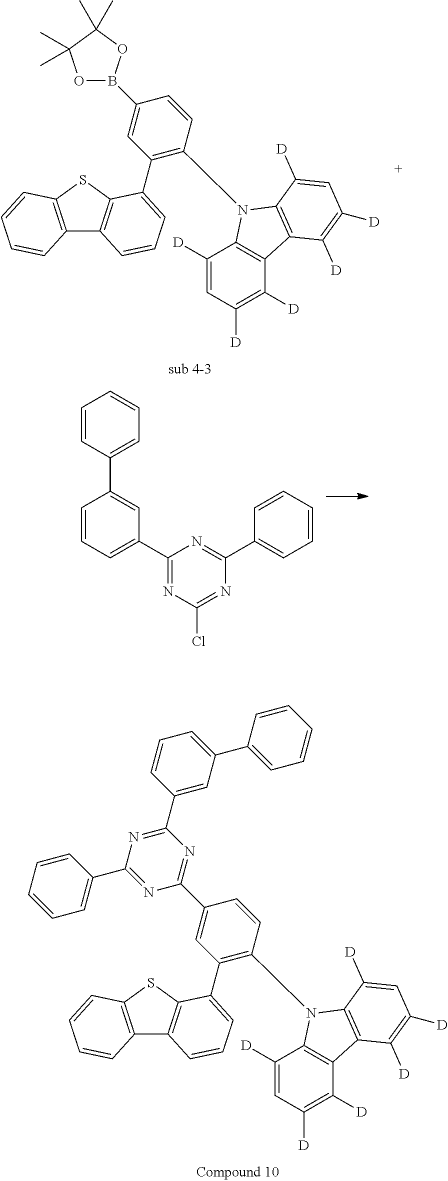 [SMILES: [2H]c1cc([2H])c2c(c1[2H])c1c([2H])c([2H])cc([2H])c1n2-c1ccc(B2OC(C)(C)C(C)(C)O2)cc1-c1cccc2c1sc1ccccc12 ; Clc1nc(-c2ccccc2)nc(-c2cccc(-c3ccccc3)c2)n1 ; [2H]c1cc([2H])c2c(c1[2H])c1c([2H])c([2H])cc([2H])c1n2-c1ccc(-c2nc(-c3ccccc3)nc(-c3cccc(-c4ccccc4)c3)n2)cc1-c1cccc2c1sc1ccccc12]

sub 4-3 (20 g, 35.9 mmol) and 2-([1,1′-biphenyl]-3-yl)-4-chloro-6-phenyl-1,3,5-triazine (12.3 g, 35.9 mmol) were added to 600 ml of tetrahydrofuran under a nitrogen atmosphere, and the mixture was stirred and refluxed. Then, potassium carbonate (14.9 g, 107.7 mmol) was dissolved in 15 ml of water and added thereto, and the mixture was sufficiently stirred and then tetrakistriphenyl-phosphinopalladium (1.2 g, 1.1 mmol) was added. After reacting for 2 hours, the reaction mixture was cooled to room temperature, and the resulting solid was filtered. The solid was added to and dissolved in 1325 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 10 (14.8 g, 56%, MS: [M+H]⁺=739.3).

### Synthesis Example 11: Preparation of Compound 11

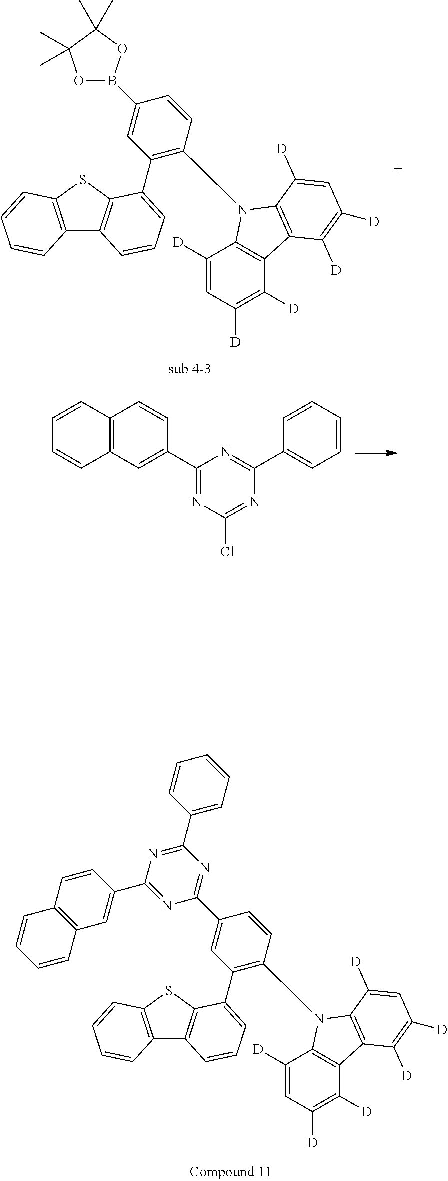 [SMILES: [2H]c1cc([2H])c2c(c1[2H])c1c([2H])c([2H])cc([2H])c1n2-c1ccc(B2OC(C)(C)C(C)(C)O2)cc1-c1cccc2c1sc1ccccc12 ; Clc1nc(-c2ccccc2)nc(-c2ccc3ccccc3c2)n1 ; [2H]c1cc([2H])c2c(c1[2H])c1c([2H])c([2H])cc([2H])c1n2-c1ccc(-c2nc(-c3ccccc3)nc(-c3ccc4ccccc4c3)n2)cc1-c1cccc2c1sc1ccccc12]

sub 4-3 (20 g, 35.9 mmol) and 2-chloro-4-(naphthalen-2-yl)-6-phenyl-1,3,5-triazine (12.3 g, 35.9 mmol) were added to 600 ml of tetrahydrofuran under a nitrogen atmosphere, and the mixture was stirred and refluxed. Then, potassium carbonate (14.9 g, 107.7 mmol) was dissolved in 15 ml of water and added thereto, and the mixture was sufficiently stirred and then tetrakistriphenyl-phosphinopalladium (1.2 g, 1.1 mmol) was added. After reacting for 2 hours, the reaction mixture was cooled to room temperature, and the resulting solid was filtered. The solid was added to and dissolved in 1278 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 11 (17.1 g, 67%, MS: [M+H]⁺=713.3).

### Synthesis Example 12: Preparation of Compound 12

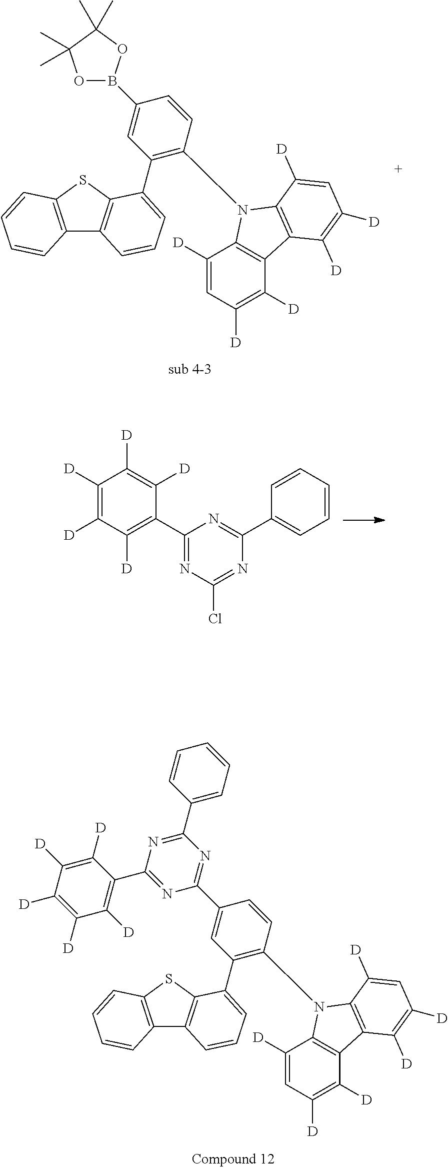 [SMILES: [2H]c1cc([2H])c2c(c1[2H])c1c([2H])c([2H])cc([2H])c1n2-c1ccc(B2OC(C)(C)C(C)(C)O2)cc1-c1cccc2c1sc1ccccc12 ; [2H]c1c([2H])c([2H])c(-c2nc(Cl)nc(-c3ccccc3)n2)c([2H])c1[2H] ; [2H]c1cc([2H])c2c(c1[2H])c1c([2H])c([2H])cc([2H])c1n2-c1ccc(-c2nc(-c3ccccc3)nc(-c3c([2H])c([2H])c([2H])c([2H])c3[2H])n2)cc1-c1cccc2c1sc1ccccc12]

sub 4-3 (20 g, 62.3 mmol) and 2-chloro-4-phenyl-6-(phenyl-d5)-1,3,5-triazine (17 g, 62.3 mmol) were added to 600 ml of tetrahydrofuran under a nitrogen atmosphere, and the mixture was stirred and refluxed. Then, potassium carbonate (25.8 g, 186.9 mmol) was dissolved in 26 ml of water and added thereto, and the mixture was sufficiently stirred and then tetrakistriphenyl-phosphinopalladium (2.2 g, 1.9 mmol) was added. After reacting for 1 hours, the reaction mixture was cooled to room temperature, and the resulting solid was filtered. The solid was added to and dissolved in 2079 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 12 (26.6 g, 64%, MS: [M+H]⁺=668.3).

### Synthesis Example 13: Preparation of Compound 13

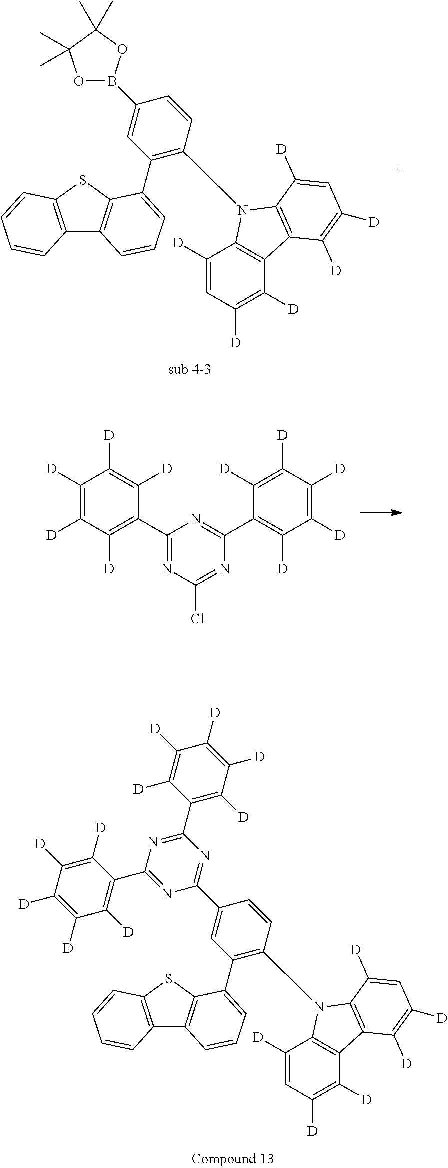 [SMILES: [2H]c1cc([2H])c2c(c1[2H])c1c([2H])c([2H])cc([2H])c1n2-c1ccc(B2OC(C)(C)C(C)(C)O2)cc1-c1cccc2c1sc1ccccc12 ; [2H]c1c([2H])c([2H])c(-c2nc(Cl)nc(-c3c([2H])c([2H])c([2H])c([2H])c3[2H])n2)c([2H])c1[2H] ; [2H]c1cc([2H])c2c(c1[2H])c1c([2H])c([2H])cc([2H])c1n2-c1ccc(-c2nc(-c3c([2H])c([2H])c([2H])c([2H])c3[2H])nc(-c3c([2H])c([2H])c([2H])c([2H])c3[2H])n2)cc1-c1cccc2c1sc1ccccc12]

sub 4-3 (20 g, 44.2 mmol) and 2-chloro-4,6-bis(phenyl-d5)-1,3,5-triazine (15.2 g, 44.2 mmol) were added to 600 mL of tetrahydrofuran under a nitrogen atmosphere, and the mixture was stirred and refluxed. Then, potassium carbonate (18.3 g, 132.7 mmol) was dissolved in 18 ml of water and added thereto, and the mixture was sufficiently stirred and then tetrakistriphenyl-phosphinopalladium (1.5 g, 1.3 mmol) was added. After reacting for 2 hours, the reaction mixture was cooled to room temperature, and the resulting solid was filtered. The solid was added to and dissolved in 1487 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 13 (20.8 g, 70%, MS: [M+H]⁺=673.3).

### Synthesis Example 14: Preparation of Compound 14

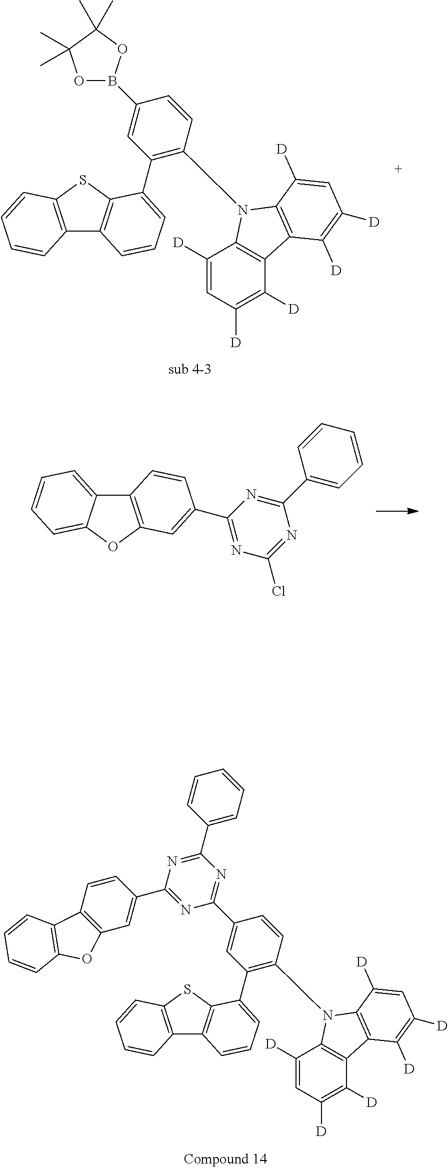 [SMILES: [2H]c1cc([2H])c2c(c1[2H])c1c([2H])c([2H])cc([2H])c1n2-c1ccc(B2OC(C)(C)C(C)(C)O2)cc1-c1cccc2c1sc1ccccc12 ; Clc1nc(-c2ccccc2)nc(-c2ccc3c(c2)oc2ccccc23)n1 ; [2H]c1cc([2H])c2c(c1[2H])c1c([2H])c([2H])cc([2H])c1n2-c1ccc(-c2nc(-c3ccccc3)nc(-c3ccc4c(c3)oc3ccccc34)n2)cc1-c1cccc2c1sc1ccccc12]

sub 4-3 (20 g, 35.9 mmol) and 2-chloro-4-(dibenzo[b, d]furan-3-yl)-6-phenyl-1,3,5-triazine (12.8 g, 35.9 mmol) were added to 600 mL of tetrahydrofuran under a nitrogen atmosphere, and the mixture was stirred and refluxed. Then, potassium carbonate (14.9 g, 107.7 mmol) was dissolved in 15 ml of water and added thereto, and the mixture was sufficiently stirred and then tetrakistriphenyl-phosphinopalladium (1.2 g, 1.1 mmol) was added. After reacting for 1 hours, the reaction mixture was cooled to room temperature, and the resulting solid was filtered. The solid was added to and dissolved in 1350 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 14 (20.5 g, 76%, MS: [M+H]⁺=753.3).

### Synthesis Example 15: Preparation of Compound 15

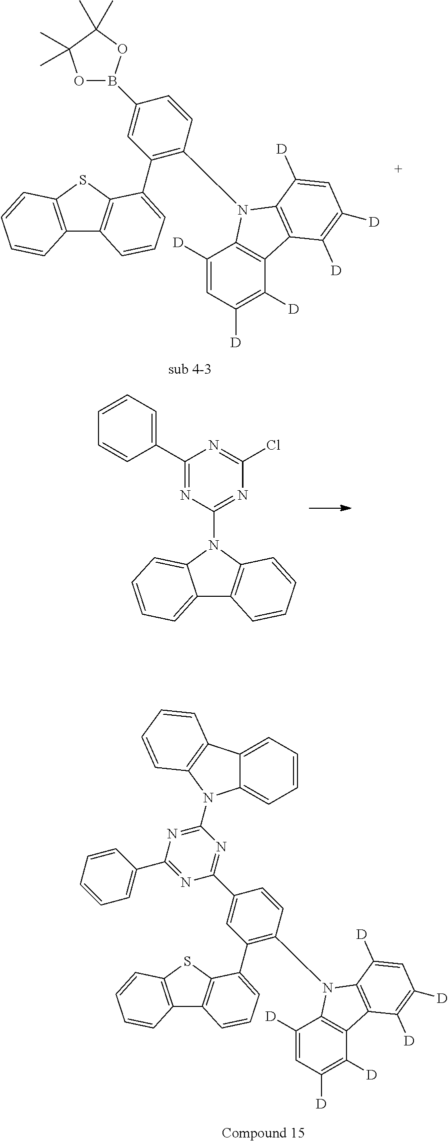 [SMILES: [2H]c1cc([2H])c2c(c1[2H])c1c([2H])c([2H])cc([2H])c1n2-c1ccc(B2OC(C)(C)C(C)(C)O2)cc1-c1cccc2c1sc1ccccc12 ; Clc1nc(-c2ccccc2)nc(-n2c3ccccc3c3ccccc32)n1 ; [2H]c1cc([2H])c2c(c1[2H])c1c([2H])c([2H])cc([2H])c1n2-c1ccc(-c2nc(-c3ccccc3)nc(-n3c4ccccc4c4ccccc43)n2)cc1-c1cccc2c1sc1ccccc12]

sub 4-3 (20 g, 35.9 mmol) and 9-(4-chloro-6-phenyl-1,3,5-triazin-2-yl)-9H-carbazole (12.8 g, 35.9 mmol) were added to 600 mL of tetrahydrofuran under a nitrogen atmosphere, and the mixture was stirred and refluxed. Then, potassium carbonate (14.9 g, 107.7 mmol) was dissolved in 15 ml of water and added thereto, and the mixture was sufficiently stirred and then tetrakistriphenyl-phosphinopalladium (1.2 g, 1.1 mmol) was added. After reacting for 2 hours, the reaction mixture was cooled to room temperature, and the resulting solid was filtered. The solid was added to and dissolved in 1348 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 15 (20.8 g, 77%, MS: [M+H]⁺=752.3).

### Synthesis Example 16: Preparation of Compound 16

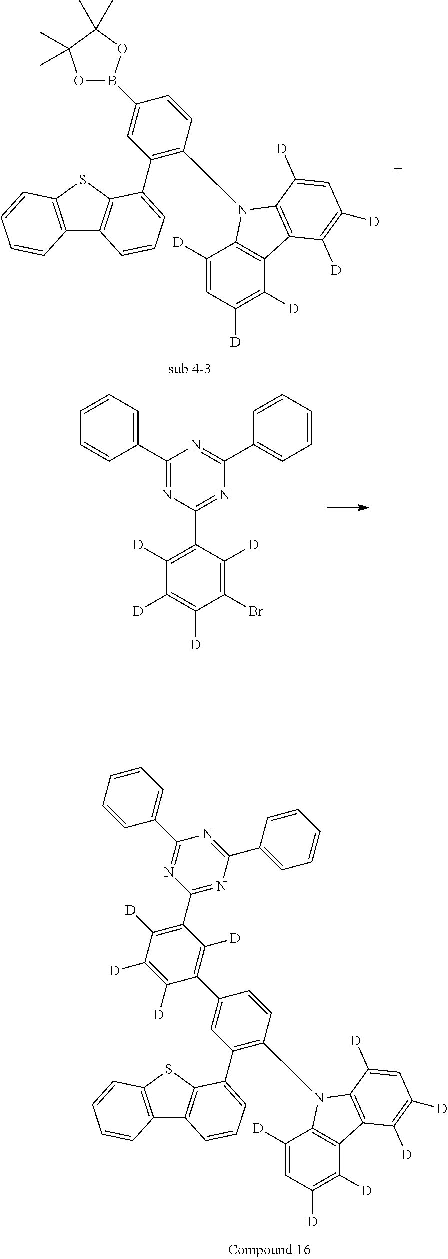 [SMILES: [2H]c1cc([2H])c2c(c1[2H])c1c([2H])c([2H])cc([2H])c1n2-c1ccc(B2OC(C)(C)C(C)(C)O2)cc1-c1cccc2c1sc1ccccc12 ; [2H]c1c([2H])c(Br)c([2H])c(-c2nc(-c3ccccc3)nc(-c3ccccc3)n2)c1[2H] ; [2H]c1cc([2H])c2c(c1[2H])c1c([2H])c([2H])cc([2H])c1n2-c1ccc(-c2c([2H])c([2H])c([2H])c(-c3nc(-c4ccccc4)nc(-c4ccccc4)n3)c2[2H])cc1-c1cccc2c1sc1ccccc12]

sub 4-3 (20 g, 35.9 mmol) and 2-(3-bromophenyl-2,4,5,6-d4)-4,6-diphenyl-1,3,5-triazine (14 g, 35.9 mmol) were added to 600 ml of tetrahydrofuran under a nitrogen atmosphere, and the mixture was stirred and refluxed. Then, potassium carbonate (14.9 g, 107.7 mmol) was dissolved in 15 mL of water and added thereto, and the mixture was sufficiently stirred and then tetrakistriphenyl-phosphinopalladium (1.2 g, 1.1 mmol) was added. After reacting for 2 hours, the reaction mixture was cooled to room temperature, and the resulting solid was filtered. The solid was added to and dissolved in 1332 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid Compound 16 (18.6 g, 70%, MS: [M+H]⁺=743.3).

### Synthesis Example 17: Preparation of Compound 17

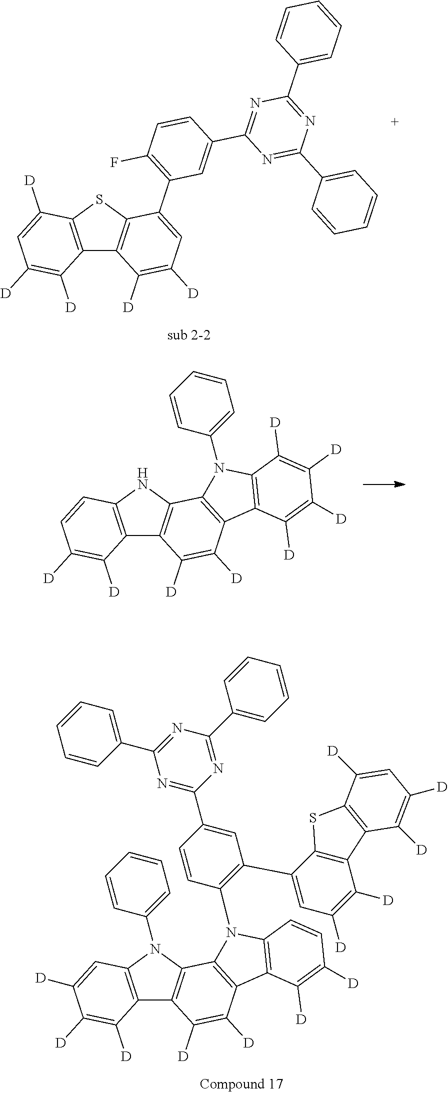 [SMILES: [2H]c1cc([2H])c2sc3c(-c4cc(-c5nc(-c6ccccc6)nc(-c6ccccc6)n5)ccc4F)cc([2H])c([2H])c3c2c1[2H] ; [2H]c1ccc2[nH]c3c(c([2H])c([2H])c4c5c([2H])c([2H])c([2H])c([2H])c5n(-c5ccccc5)c34)c2c1[2H] ; [2H]c1cc2c(c([2H])c1[2H])c1c([2H])c([2H])c3c4c([2H])c([2H])ccc4n(-c4ccc(-c5nc(-c6ccccc6)nc(-c6ccccc6)n5)cc4-c4cc([2H])c([2H])c5c4sc4c([2H])cc([2H])c([2H])c45)c3c1n2-c1ccccc1]

A solid Compound sub 2-2 was prepared in the same manner as in Synthesis Example 4.

Next, sub 2-2 (50 g, 97.2 mmol) and 11-phenyl-11,12-dihydroindolo[2,3-a]carbazole-3,4,5,6,7,8,9-d7 (33.1 g, 97.2 mmol) were added to 1000 ml of xylene under a nitrogen atmosphere, and the mixture was stirred and refluxed. Then, sodium tert-butoxide (28 g, 291.7 mmol) was added thereto, and sufficiently stirred and then bis(tri-tert-butylphosphine) palladium (1.5 g, 2.9 mmol) was added. After reacting for 2 hours, the reaction mixture was cooled to room temperature, and the resulting solid was filtered. The solid was added to and dissolved in 2370 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid compound 17 (45 g, 57%, MS: [M+H]⁺=813.2).

### Synthesis Example 18: Preparation of Compound 18

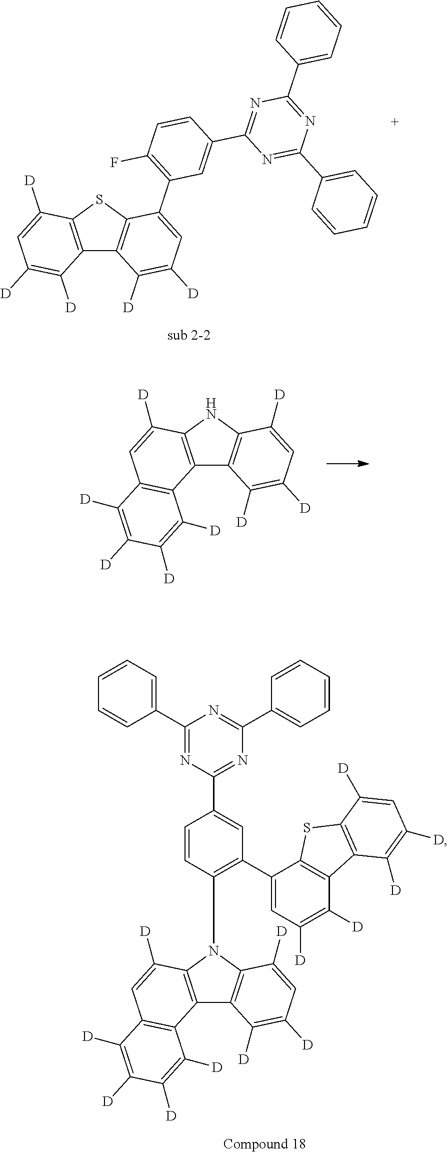

A solid Compound sub 2-2 was prepared in the same manner as in Synthesis Example 4.

Next, sub 2-2 (50 g, 97.1 mmol) and 7H-benzo[c]carbazole-1,2,3,4,6,8,10,11-d8 (21.9 g, 97.1 mmol) were added to 1000 mL of xylene under a nitrogen atmosphere, and the mixture was stirred and refluxed. Then, sodium tert-butoxide (28 g, 291.2 mmol) was added thereto, and sufficiently stirred and then bis(tri-tert-butylphosphine) palladium (1.5 g, 2.9 mmol) was added. After reacting for 1 hours, the reaction mixture was cooled to room temperature, and the resulting solid was filtered. The solid was added to and dissolved in 2033 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid compound 18 (37.3 g, 55%, MS: [M+H]⁺=699.3).

### Synthesis Example 19: Preparation of Compound 19

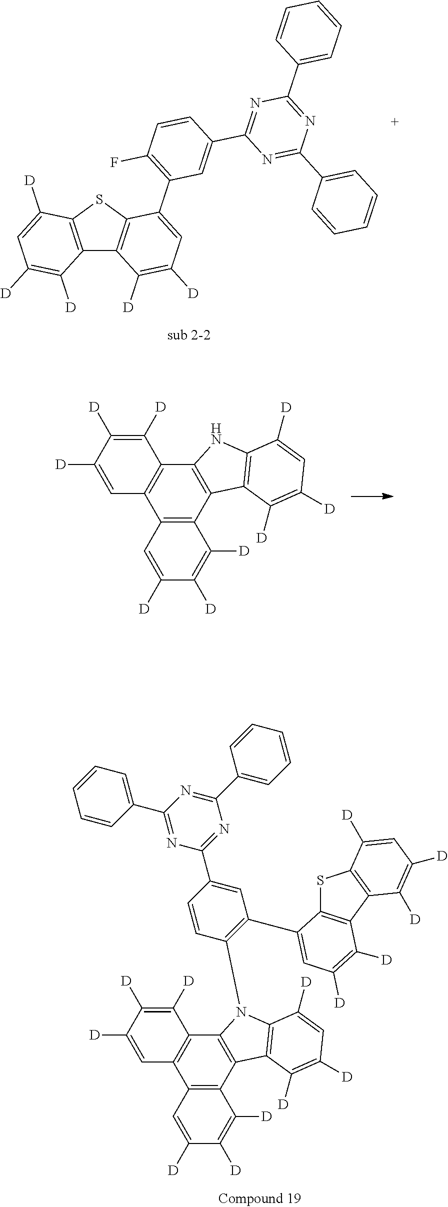 [SMILES: [2H]c1cc([2H])c2sc3c(-c4cc(-c5nc(-c6ccccc6)nc(-c6ccccc6)n5)ccc4F)cc([2H])c([2H])c3c2c1[2H] ; [2H]c1cc2c3cc([2H])c([2H])c([2H])c3c3c([nH]c4c([2H])cc([2H])c([2H])c43)c2c([2H])c1[2H] ; [2H]c1cc2c3cc([2H])c([2H])c([2H])c3c3c(c2c([2H])c1[2H])c1c([2H])c([2H])cc([2H])c1n3-c1ccc(-c2nc(-c3ccccc3)nc(-c3ccccc3)n2)cc1-c1cc([2H])c([2H])c2c1sc1c([2H])cc([2H])c([2H])c12]

A solid compound sub 2-2 was prepared in the same manner as in Synthesis Example 4.

Next, sub 2-2 (50 g, 96.9 mmol) and 9H-dibenzo[a,c]carbazole-1,2,3,6,7,8,10,12,13-d9 (26.8 g, 96.9 mmol) were added to 1000 ml of xylene under a nitrogen atmosphere, and the mixture was stirred and refluxed. Then, sodium tert-butoxide (27.9 g, 290.6 mmol) was added thereto, and sufficiently stirred and then bis(tri-tert-butylphosphine) palladium (1.5 g, 2.9 mmol) was added. After reacting for 3 hours, the reaction mixture was cooled to room temperature, and the resulting solid was filtered. The solid was added to and dissolved in 2178 mL of chloroform, washed twice with water, and then the organic layer was separated, anhydrous magnesium sulfate was added, stirred, then filtered, and the filtrate was distilled under reduced pressure. The concentrated compound was purified through a silica column using chloroform and ethyl acetate to prepare a white solid compound 19 (46.5 g, 64%, MS: [M+H]⁺=750.3).

### EXAMPLES

### Example 1

A glass substrate on which a thin film of ITO (indium tin oxide) was coated in a thickness of 1,300 Å was put into distilled water containing a detergent dissolved therein and ultrasonically cleaned. In this case, the detergent used was a product commercially available from Fischer Co. and the distilled water was one which had been twice filtered by using a filter commercially available from Millipore Co. The ITO was cleaned for 30 minutes, and ultrasonic cleaning was then repeated twice for 10 minutes by using distilled water. After the cleaning with distilled water was completed, the substrate was ultrasonically washed with the solvents of isopropyl alcohol, acetone, and methanol, and dried, after which it was transported to a plasma cleaner. Then, the substrate was cleaned with oxygen plasma for 5 minutes, and then transferred to a vacuum evaporator.

On the ITO transparent electrode thus prepared, the following Compound HI-1 was thermally vacuum-deposited to a thickness of 50 Å to form a hole injection layer. The following Compound HT-1 was thermally vacuum-deposited to a thickness of 250 Å on the hole injection layer to form hole transport layer, and the following Compound HT-2 was vacuum deposited on the HT-1 deposited layer to a thickness of 50 Å to form an electron blocking layer.

Then, Compound 1 prepared in the previous Synthesis Example 1, the following Compound YGH-1, and the following phosphorescence dopant YGD-1 were vacuum deposited as a light emitting layer at a weight ratio of 44:44:12 on the HT-2 deposited layer to form a light emitting layer with a thickness of 400 Å.

The following Compound ET-1 was vacuum deposited on the light emitting layer to a thickness of 250 Å to form an electron transport layer, and the following Compound ET-2 and LiF were vacuum deposited at a weight ratio of 98:2 on the electron transport layer to form an electron injection layer with a film thickness of 100 Å. Aluminum was deposited on the electron injection layer to a thickness of 1,000 Å to form a cathode.

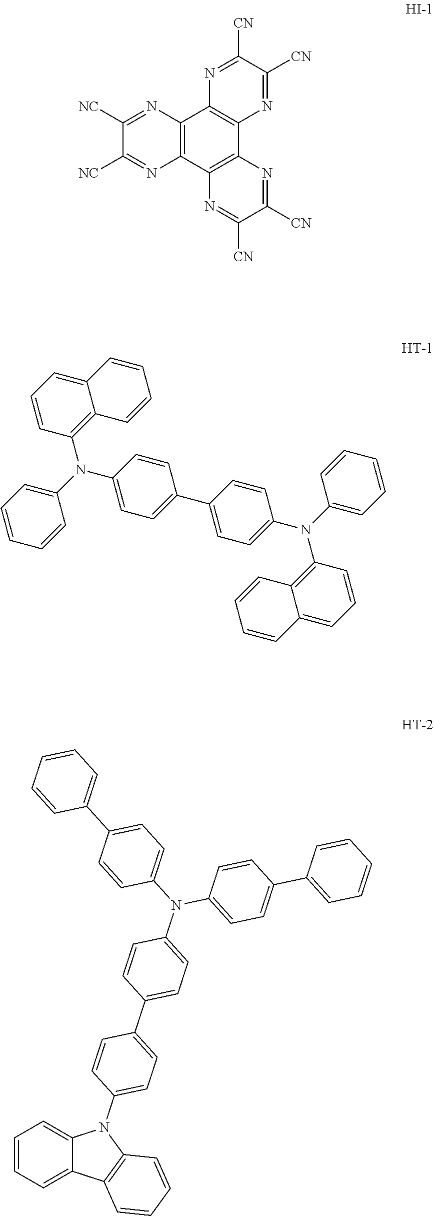 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1 ; c1ccc(-c2ccc(N(c3ccc(-c4ccccc4)cc3)c3ccc(-c4ccc(-n5c6ccccc6c6ccccc65)cc4)cc3)cc2)cc1]

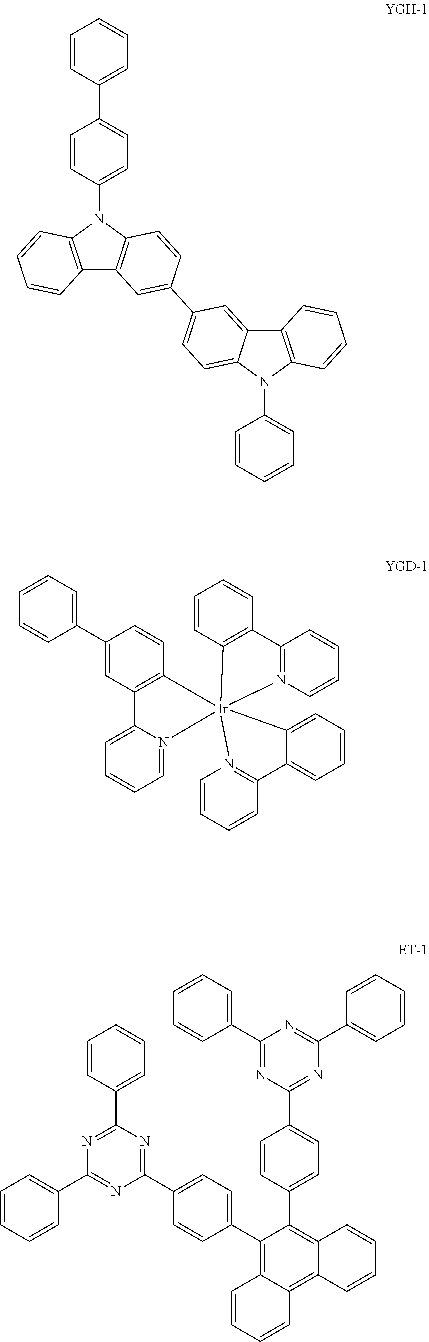

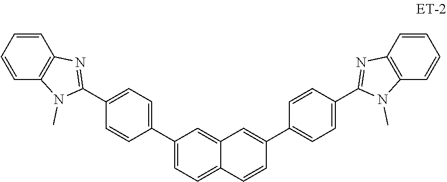 [SMILES: Cn1c(-c2ccc(-c3ccc4ccc(-c5ccc(-c6nc7ccccc7n6C)cc5)cc4c3)cc2)nc2ccccc21]

In the above-mentioned processes, the deposition rate of the organic material was maintained at 0.4˜0.7 Å/sec, the deposition rate of aluminum was maintained at 2 Å/sec, and the degree of vacuum during deposition was maintained at 1×10⁻⁷˜ 5×10⁻⁸ torr.

### Examples 2 to 19

The organic light emitting devices of Examples 2 to 19 were manufactured in the same manner as in Example 1, except that as one of the host materials for the light emitting layer in Example 1, the Compound shown in Table 1 below was used instead of the Compound 1 of Synthetic Example 1.

At this time, the structures of the compounds used in Examples 1 to 19 are as follows.

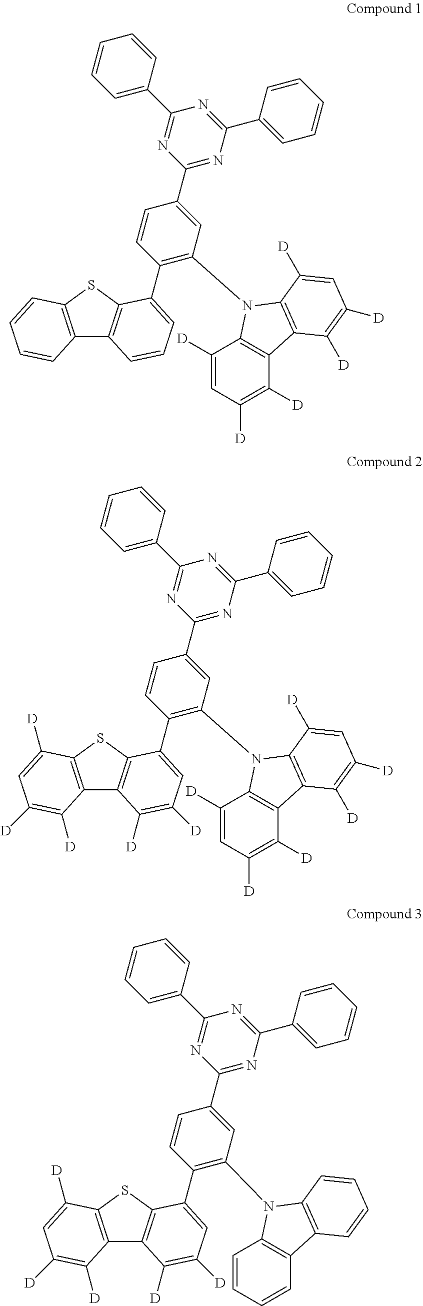 [SMILES: [2H]c1cc([2H])c2c(c1[2H])c1c([2H])c([2H])cc([2H])c1n2-c1cc(-c2nc(-c3ccccc3)nc(-c3ccccc3)n2)ccc1-c1cccc2c1sc1ccccc12 ; [2H]c1cc([2H])c2sc3c(-c4ccc(-c5nc(-c6ccccc6)nc(-c6ccccc6)n5)cc4-n4c5c([2H])cc([2H])c([2H])c5c5c([2H])c([2H])cc([2H])c54)cc([2H])c([2H])c3c2c1[2H] ; [2H]c1cc([2H])c2sc3c(-c4ccc(-c5nc(-c6ccccc6)nc(-c6ccccc6)n5)cc4-n4c5ccccc5c5ccccc54)cc([2H])c([2H])c3c2c1[2H]]

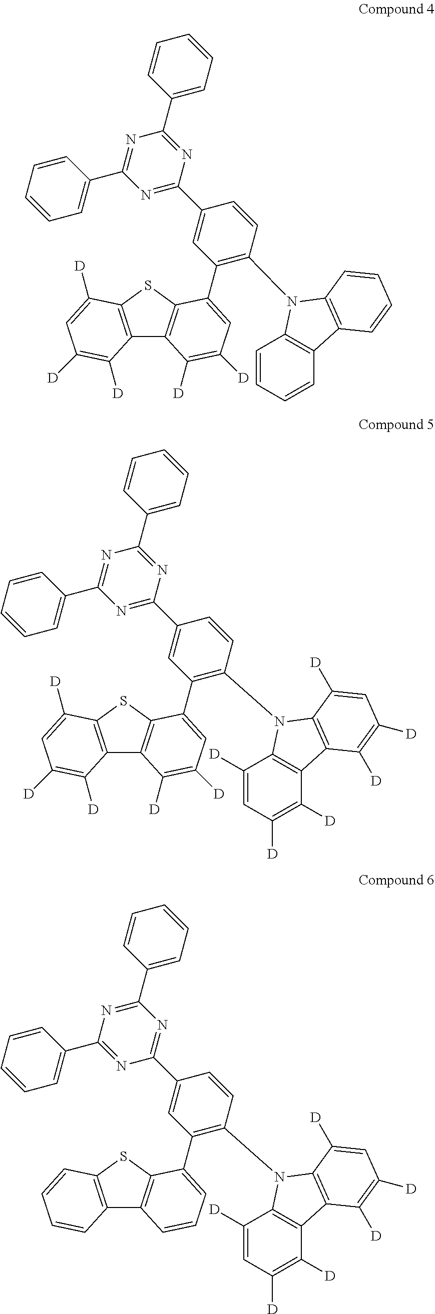 [SMILES: [2H]c1cc([2H])c2sc3c(-c4cc(-c5nc(-c6ccccc6)nc(-c6ccccc6)n5)ccc4-n4c5ccccc5c5ccccc54)cc([2H])c([2H])c3c2c1[2H] ; [2H]c1cc([2H])c2sc3c(-c4cc(-c5nc(-c6ccccc6)nc(-c6ccccc6)n5)ccc4-n4c5c([2H])cc([2H])c([2H])c5c5c([2H])c([2H])cc([2H])c54)cc([2H])c([2H])c3c2c1[2H] ; [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1sc1ccccc12]

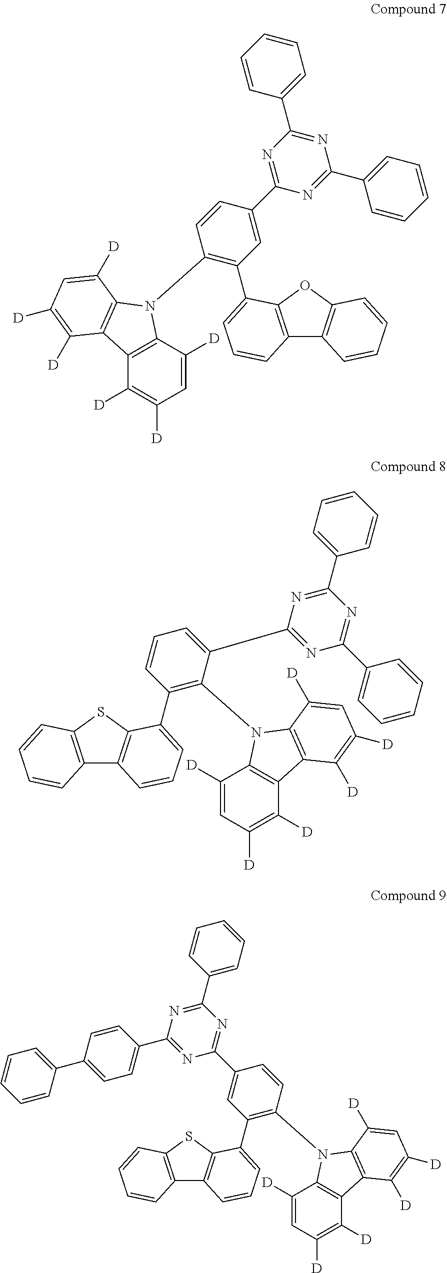 [SMILES: [2H]c1cc([2H])c2c(c1[2H])c1c([2H])c([2H])cc([2H])c1n2-c1ccc(-c2nc(-c3ccccc3)nc(-c3ccccc3)n2)cc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3ccccc3)n2)cccc1-c1cccc2c1sc1ccccc12 ; [2H]c1cc([2H])c2c(c1[2H])c1c([2H])c([2H])cc([2H])c1n2-c1ccc(-c2nc(-c3ccccc3)nc(-c3ccc(-c4ccccc4)cc3)n2)cc1-c1cccc2c1sc1ccccc12]

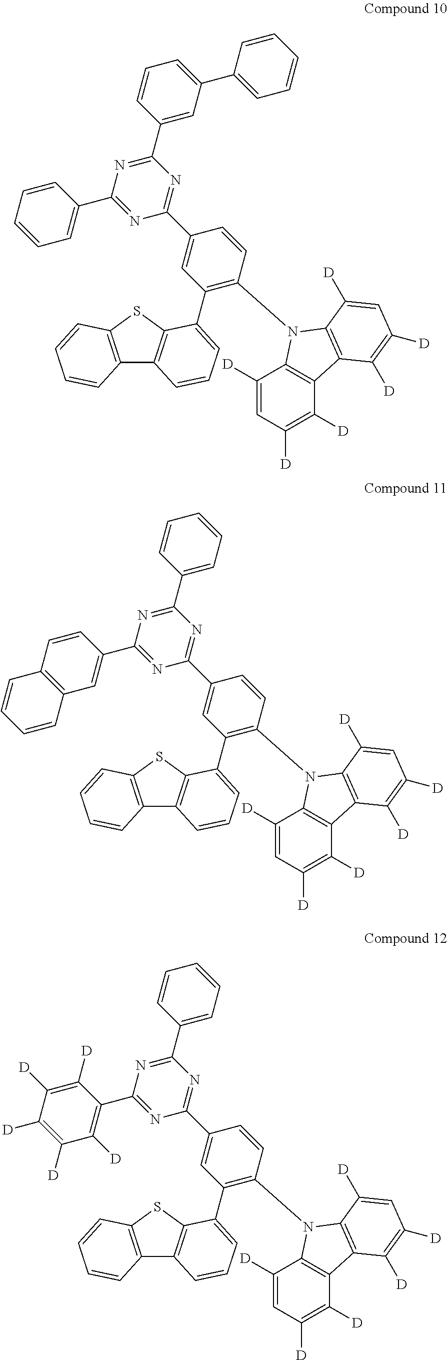 [SMILES: [2H]c1cc([2H])c2c(c1[2H])c1c([2H])c([2H])cc([2H])c1n2-c1ccc(-c2nc(-c3ccccc3)nc(-c3cccc(-c4ccccc4)c3)n2)cc1-c1cccc2c1sc1ccccc12 ; [2H]c1cc([2H])c2c(c1[2H])c1c([2H])c([2H])cc([2H])c1n2-c1ccc(-c2nc(-c3ccccc3)nc(-c3ccc4ccccc4c3)n2)cc1-c1cccc2c1sc1ccccc12 ; [2H]c1cc([2H])c2c(c1[2H])c1c([2H])c([2H])cc([2H])c1n2-c1ccc(-c2nc(-c3ccccc3)nc(-c3c([2H])c([2H])c([2H])c([2H])c3[2H])n2)cc1-c1cccc2c1sc1ccccc12]

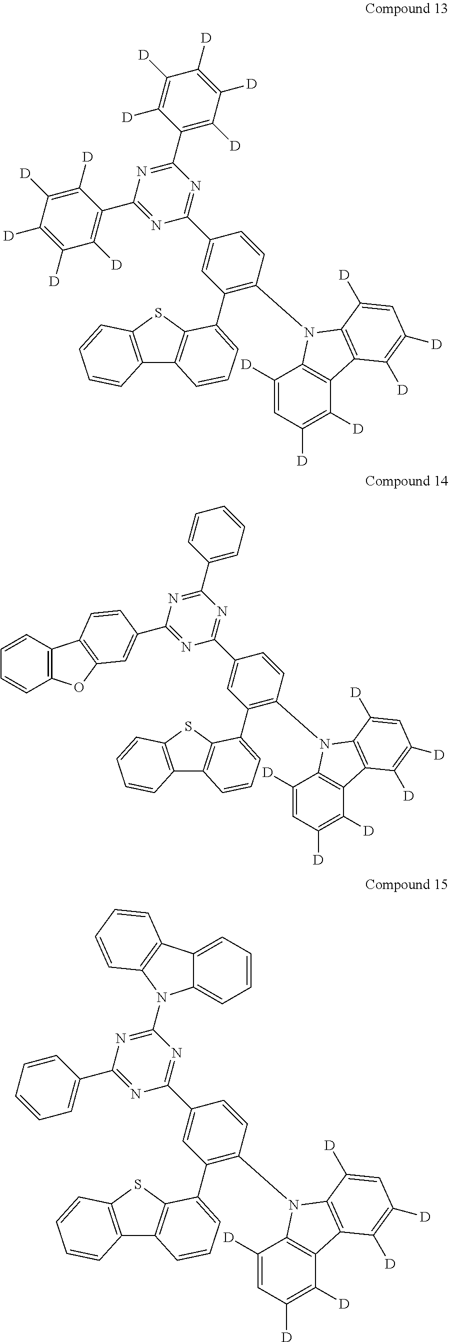 [SMILES: [2H]c1cc([2H])c2c(c1[2H])c1c([2H])c([2H])cc([2H])c1n2-c1ccc(-c2nc(-c3c([2H])c([2H])c([2H])c([2H])c3[2H])nc(-c3c([2H])c([2H])c([2H])c([2H])c3[2H])n2)cc1-c1cccc2c1sc1ccccc12 ; [2H]c1cc([2H])c2c(c1[2H])c1c([2H])c([2H])cc([2H])c1n2-c1ccc(-c2nc(-c3ccccc3)nc(-c3ccc4c(c3)oc3ccccc34)n2)cc1-c1cccc2c1sc1ccccc12 ; [2H]c1cc([2H])c2c(c1[2H])c1c([2H])c([2H])cc([2H])c1n2-c1ccc(-c2nc(-c3ccccc3)nc(-n3c4ccccc4c4ccccc43)n2)cc1-c1cccc2c1sc1ccccc12]

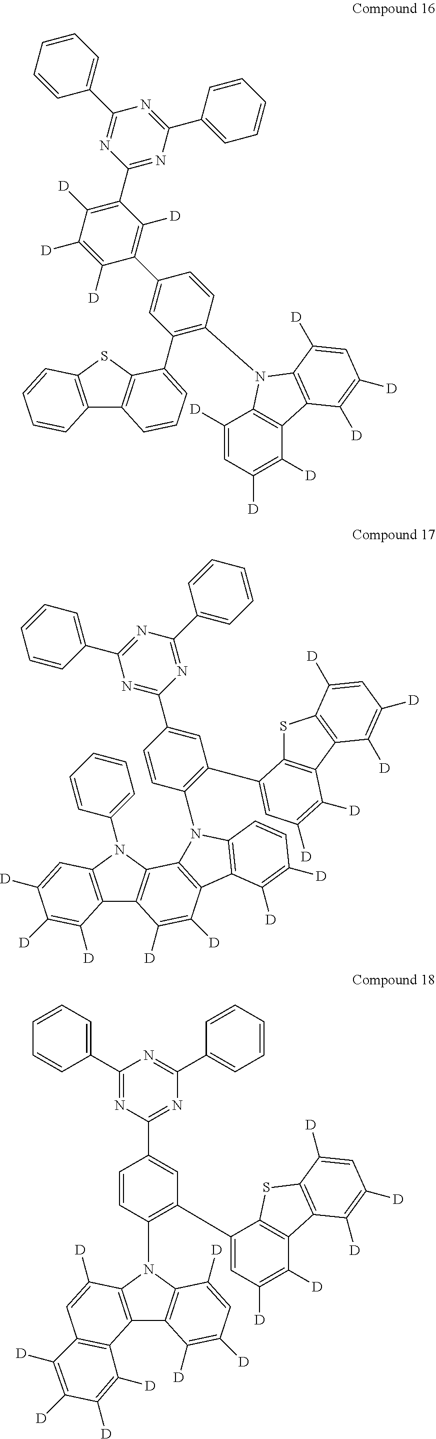 [SMILES: [2H]c1cc([2H])c2c(c1[2H])c1c([2H])c([2H])cc([2H])c1n2-c1ccc(-c2c([2H])c([2H])c([2H])c(-c3nc(-c4ccccc4)nc(-c4ccccc4)n3)c2[2H])cc1-c1cccc2c1sc1ccccc12 ; [2H]c1cc2c(c([2H])c1[2H])c1c([2H])c([2H])c3c4c([2H])c([2H])ccc4n(-c4ccc(-c5nc(-c6ccccc6)nc(-c6ccccc6)n5)cc4-c4cc([2H])c([2H])c5c4sc4c([2H])cc([2H])c([2H])c45)c3c1n2-c1ccccc1 ; [2H]c1cc([2H])c2sc3c(-c4cc(-c5nc(-c6ccccc6)nc(-c6ccccc6)n5)ccc4-n4c5c([2H])cc([2H])c([2H])c5c5c6c([2H])c([2H])c([2H])c([2H])c6cc([2H])c54)cc([2H])c([2H])c3c2c1[2H]]

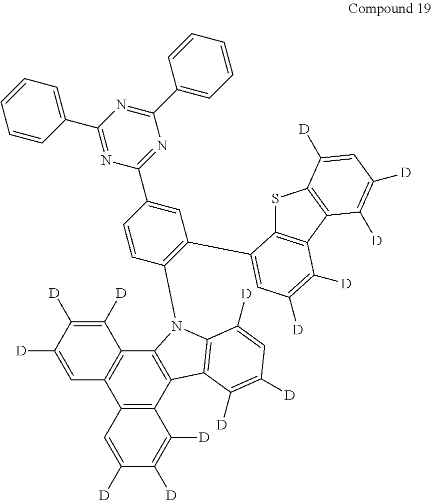 [SMILES: [2H]c1cc2c3cc([2H])c([2H])c([2H])c3c3c(c2c([2H])c1[2H])c1c([2H])c([2H])cc([2H])c1n3-c1ccc(-c2nc(-c3ccccc3)nc(-c3ccccc3)n2)cc1-c1cc([2H])c([2H])c2c1sc1c([2H])cc([2H])c([2H])c12]

### Comparative Examples 1 to 5

The organic light emitting devices of Comparative Examples 1 to 5 were manufactured in the same manner as in Example 1, except that as one of the host materials for the light emitting layer in Example 1, the Compound shown in Table 1 below was used instead of the Compound 1 of Synthetic Example 1. At this time, the Compounds CE1 to CE5 of Table 1 below are as follows.

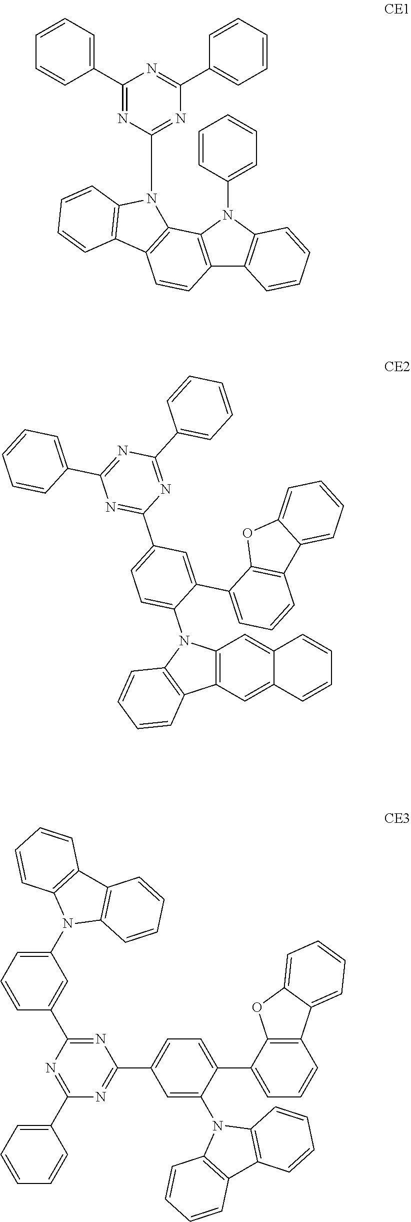 [SMILES: c1ccc(-c2nc(-c3ccccc3)nc(-n3c4ccccc4c4ccc5c6ccccc6n(-c6ccccc6)c5c43)n2)cc1 ; c1ccc(-c2nc(-c3ccccc3)nc(-c3ccc(-n4c5ccccc5c5cc6ccccc6cc54)c(-c4cccc5c4oc4ccccc45)c3)n2)cc1 ; c1ccc(-c2nc(-c3cccc(-n4c5ccccc5c5ccccc54)c3)nc(-c3ccc(-c4cccc5c4oc4ccccc45)c(-n4c5ccccc5c5ccccc54)c3)n2)cc1]

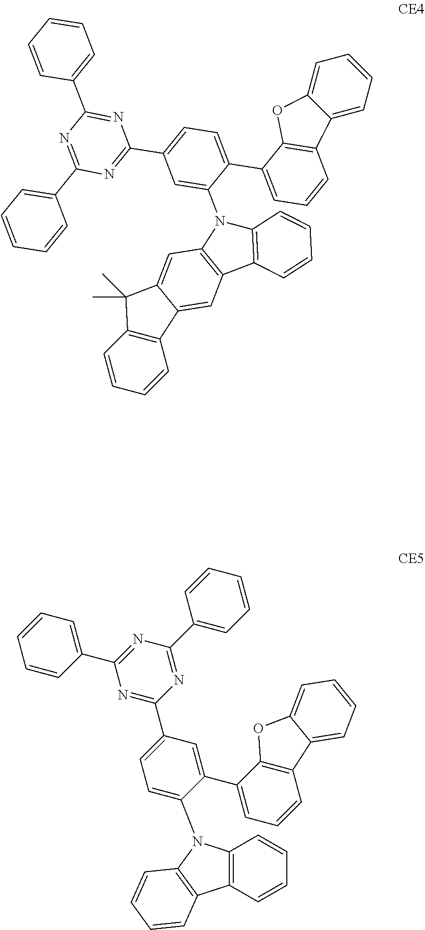 [SMILES: CC1(C)c2ccccc2-c2cc3c4ccccc4n(-c4cc(-c5nc(-c6ccccc6)nc(-c6ccccc6)n5)ccc4-c4cccc5c4oc4ccccc45)c3cc21 ; c1ccc(-c2nc(-c3ccccc3)nc(-c3ccc(-n4c5ccccc5c5ccccc54)c(-c4cccc5c4oc4ccccc45)c3)n2)cc1]

### Experimental Example 1: Evaluation of Device Characteristics

The voltage and efficiency of the organic light emitting devices manufactured in Examples and Comparative Examples were measured at a current density of 10 mA/cm², and the lifetime was measured at current density of 50 mA/cm². The results are shown in Table 1 below. At this time, lifetime LT95 means the time required for the luminance to be reduced to 95% of the initial luminance.

TABLE 1

| | | | | Color | Lifetime(h) |
|---|---|---|---|---|---|
| | | Voltage (V) | Efficiency(Cd/A) | coordinate | (LT₉₅ at |
| | Compound | (@10 mA/cm²) | (@10 mA/cm²) | (x, y) | 50 mA/cm²) |
| Example 1 | Compound | 3.1 | 80.5 | 0.44, | 209.4 |
| | 1 | | | 0.53 | |
| Example 2 | Compound | 3.2 | 80.3 | 0.432, | 230 |
| | 2 | | | 0.549 | |
| Example 3 | Compound | 3.2 | 80.3 | 0.432, | 211.3 |
| | 3 | | | 0.549 | |
| Example 4 | Compound | 3.9 | 81.6 | 0.432, | 213.3 |
| | 4 | | | 0.541 | |
| Example 5 | Compound | 3.9 | 81.7 | 0.434, | 250 |
| | 5 | | | 0.53 | |
| Example 6 | Compound | 3.8 | 81.9 | 0.439, | 214.4 |
| | 6 | | | 0.531 | |
| Example 7 | Compound | 3.6 | 82.4 | 0.431, | 195.5 |
| | 7 | | | 0.533 | |
| Example 8 | Compound | 3.7 | 82.4 | 0.44, | 217 |
| | 8 | | | 0.535 | |
| Example 9 | Compound | 3.8 | 82.8 | 0.44, | 217.8 |
| | 9 | | | 0.532 | |
| Example 10 | Compound | 4 | 82.9 | 0.432, | 218.5 |
| | 10 | | | 0.55 | |
| Example 11 | Compound | 3.8 | 83.3 | 0.439, | 219.7 |
| | 11 | | | 0.547 | |
| Example 12 | Compound | 3.8 | 83.7 | 0.43, | 220.5 |
| | 12 | | | 0.534 | |
| Example 13 | Compound | 3.9 | 84.1 | 0.436, | 221.9 |
| | 13 | | | 0.549 | |
| Example 14 | Compound | 4 | 84.1 | 0.44, | 222.5 |
| | 14 | | | 0.536 | |
| Example 15 | Compound | 3.8 | 84.3 | 0.449, | 224.2 |
| | 15 | | | 0.542 | |
| Example 16 | Compound | 3.7 | 85.7 | 0.439, | 174.2 |
| | 16 | | | 0.549 | |
| Example 17 | Compound | 3.8 | 84.7 | 0.442, | 215.5 |
| | 17 | | | 0.536 | |
| Example 18 | Compound | 3.9 | 80.5 | 0.434, | 204.5 |
| | 18 | | | 0.531 | |
| Example 19 | Compound | 4.1 | 80.3 | 0.439, | 200.1 |
| | 19 | | | 0.549 | |
| Comparative | CE1 | 4 | 70 | 0.441, | 100 |
| Example 1 | | | | 0.545 | |
| Comparative | CE2 | 4.2 | 58 | 0.431, | 45 |
| Example 2 | | | | 0.537 | |
| Comparative | CE3 | 4.4 | 77 | 0.44, | 160 |
| Example 3 | | | | 0.545 | |
| Comparative | CE4 | 4.3 | 75 | 0.446, | 145 |
| Example 4 | | | | 0.536 | |
| Comparative | CE5 | 3.6 | 82.4 | 0.431, | 130 |
| Example 5 | | | | 0.533 | |

As shown in Table 1, it can be confirmed that the organic light emitting devices of the Examples using the compound of Chemical Formula 1 as a host material for the light emitting layer exhibit an equivalent or higher level of efficiency and a remarkable lifetime characteristic as compared to the organic light emitting device of the Comparative Examples using a compound having a structure different therefrom. Therefore, considering that the luminous efficiency and lifetime characteristics of organic light emitting devices generally have a trade-off relationship, it can be confirmed that the compound of the present disclosure can improve the characteristics of the organic light emitting devices as compared to the compounds of the Comparative Examples.

[Description of Symbols]

| | |
|---|---|
| 1: substrate | 2: anode |
| 3: light emitting layer | 4: cathode |
| 5: hole injection layer | 6: hole transport layer |
| 7: electron blocking layer | 8: electron transport layer |
| 9: electron injection layer | |

1. A compound of Chemical Formula 1: 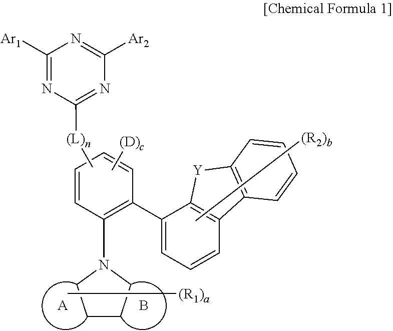
wherein, in Chemical Formula 1:
Y is O or S;
L is a single bond, a substituted or unsubstituted C₆₋₆₀ arylene, or a substituted or unsubstituted C₂₋₆₀ heteroarylene containing one or more heteroatoms of N, O and S;
n is 1, 2, or 3;
Ar₁ and Ar₂ are each independently a substituted or unsubstituted C₆₋₆₀ aryl or a substituted or unsubstituted C₂₋₆₀ heteroaryl containing one or more heteroatoms of N, O and S;
A and B are each independently a C₆₋₆₀ aromatic ring or a C₂₋₆₀ heteroaromatic ring containing one or more heteroatoms of N, O and S which is fused with an adjacent pentagonal ring;
R₁ and R₂ are each independently deuterium, a substituted or unsubstituted C₆₋₆₀ aryl, or a substituted or unsubstituted C₂₋₆₀ heteroaryl containing one or more heteroatoms of N, O and S;
D is deuterium;
a is an integer of 0 to 12;
b is an integer of 0 to 7; and
c is an integer of 0 to 3,
with the proviso that at least one of R₁ and R₂ is deuterium, and a+b is 1 or more.

2. The compound according to claim 1, wherein:
L is a single bond; phenylene that is unsubstituted or substituted with 1 to 4 deuteriums; biphenyldiyl that is unsubstituted or substituted with 1 to 8 deuteriums; terphenyldiyl that is unsubstituted or substituted with 1 to 12 deuteriums; naphthylene that is unsubstituted or substituted with 1 to 6 deuterium; anthracenylene that is unsubstituted or substituted with 1 to 8 deuteriums; dibenzofuranylene that is unsubstituted or substituted with 1 to 6 deuteriums; or dibenzothiophenylene that is unsubstituted or substituted with 1 to 6 deuteriums.

3. The compound according to claim 1, wherein:
-(L)n—is a single bond or any one selected from the group consisting of: 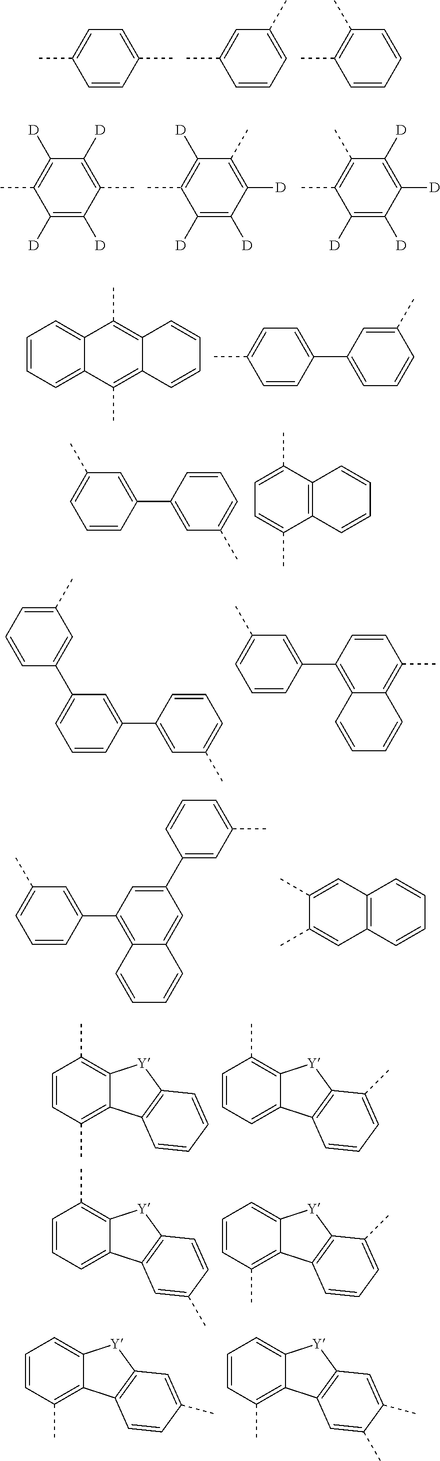 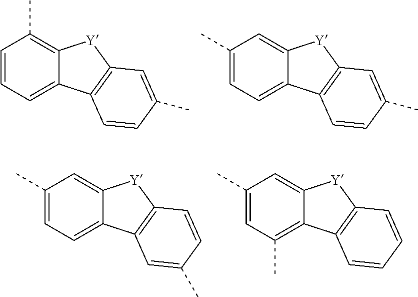
wherein Y′ is O or S.

4. The compound according to claim 1, wherein:
Ar₁ and Ar₂ are each independently phenyl, biphenylyl, naphthyl, phenanthryl, dibenzofuranyl, dibenzothiophenyl, carbazolyl, or phenylcarbazolyl,
wherein Ar₁ and Ar₂ are unsubstituted or substituted with at least one deuterium.

5. The compound according to claim 1, wherein:
Ar₁ and Ar₂ are each independently any one selected from the group consisting of:

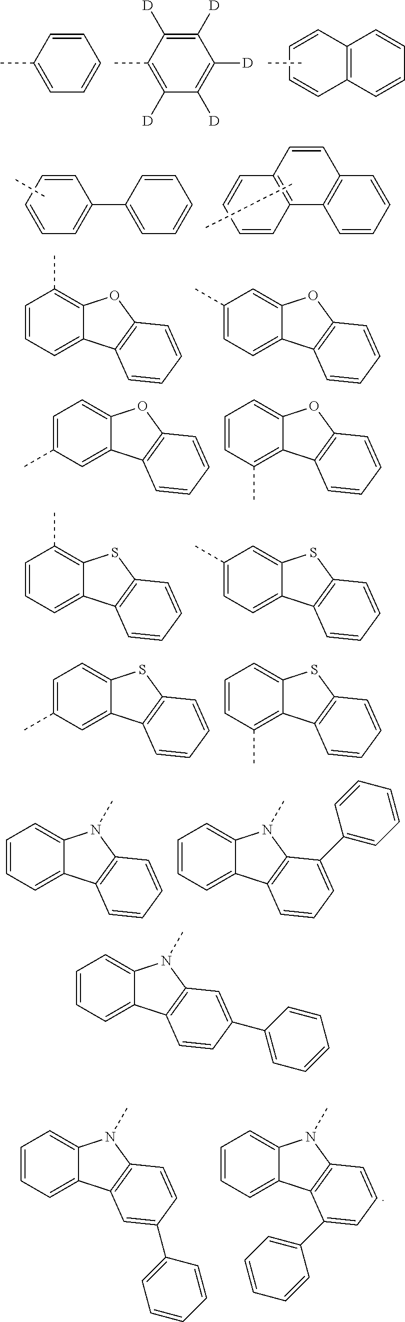

6. The compound according to claim 1, wherein:
A and B are each independently a benzene ring, a naphthalene ring, a phenanthrene ring, or a carbazole ring.

7. The compound according to claim 1, wherein:
R₁ and R₂ are each independently deuterium or phenyl that is unsubstituted or substituted with 1 to 5 deuteriums.

8. The compound according to claim 1, wherein:
a is an integer of 1 to 12, and b is 0; or
a is 0, and b is an integer of 1 to 7; or
a is an integer of 1 to 12, and b is an integer of 1 to 7.

9. The compound according to claim 1, wherein: 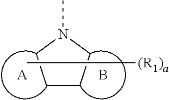
is any one of the following Chemical Formulas 2a to 2o: 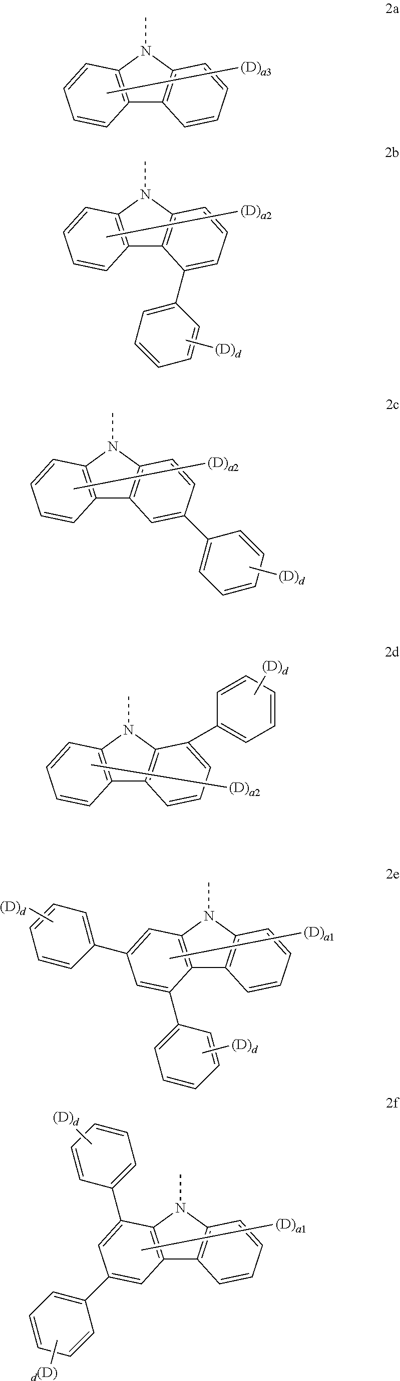 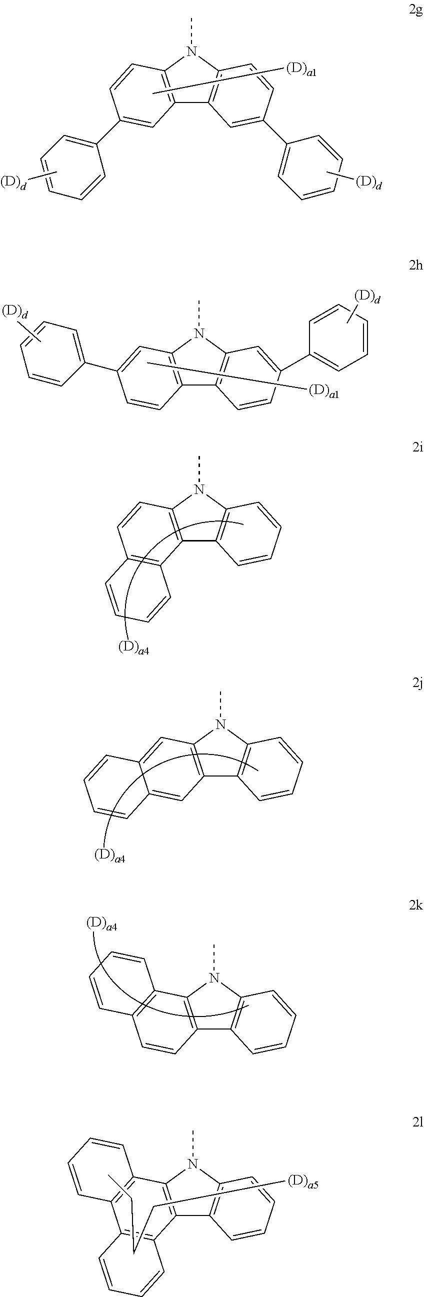 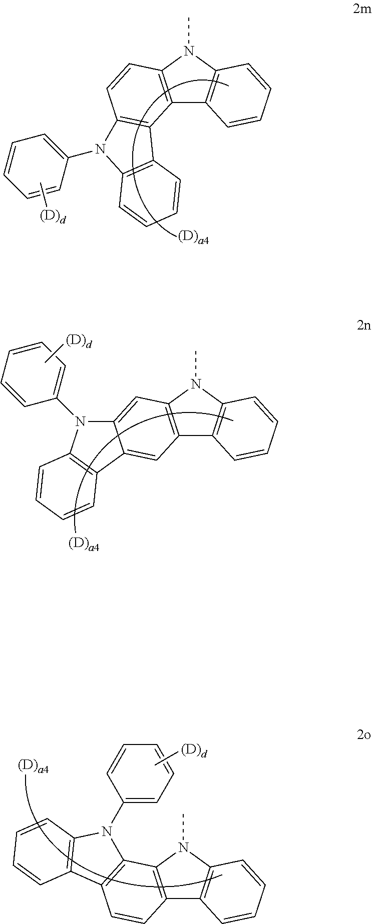
wherein, in Chemical Formulas 2a to 2o:
D is deuterium;
each d is independently an integer of 0 to 5;
a1 is an integer of 0 to 6;
a2 is an integer of 0 to 7;
a3 is an integer of 0 to 8;
a4 is an integer of 0 to 10; and
a5 is an integer of 0 to 12,
with the proviso that a1+b is 1 or more, or a2+b is 1 or more, or a3+b is 1 or more, or a4+b is 1 or more, or a5+b is 1 or more.

10. The compound according to claim 1, wherein: 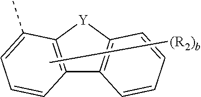
is any one of the following Chemical Formulas 3a to 3j: 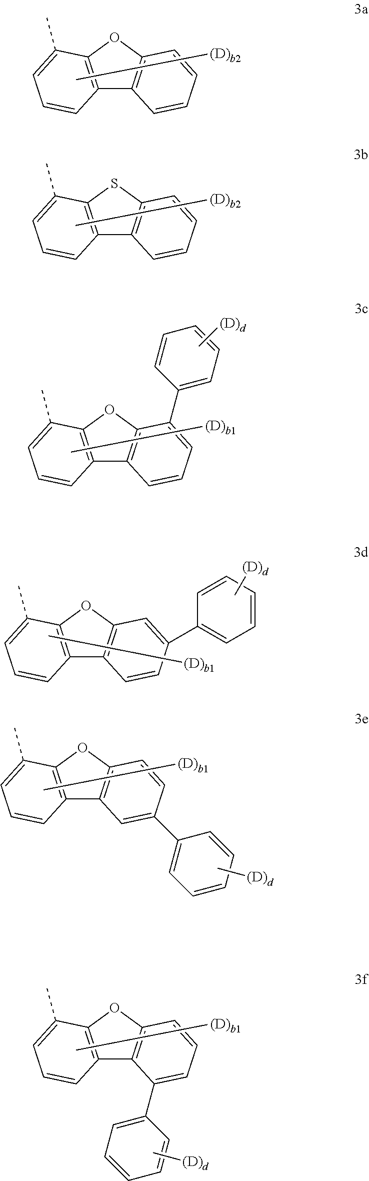 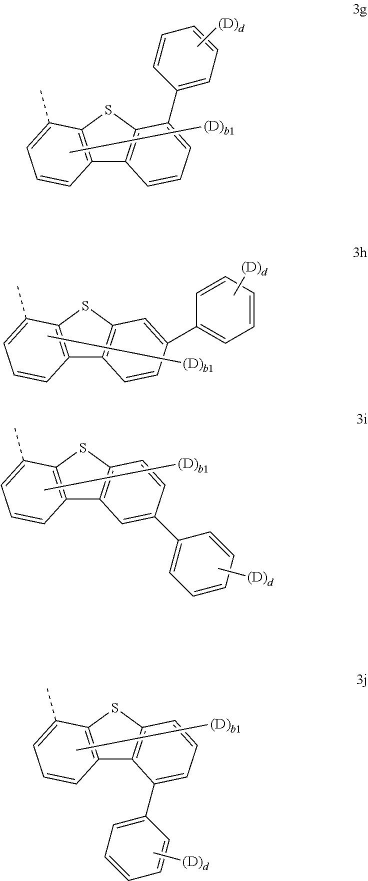
wherein in Chemical Formulas 3a to 3j:
D is deuterium;
d is an integer of 0 to 5;
b1 is an integer of 0 to 6;
b2 is an integer of 0 to 7;
with the proviso that a+b1 is 1 or more, or a+b2 is 1 or more.

11. The compound according to claim 1, wherein: 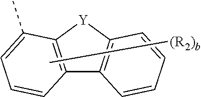
is any one substituent selected from the group consisting of:

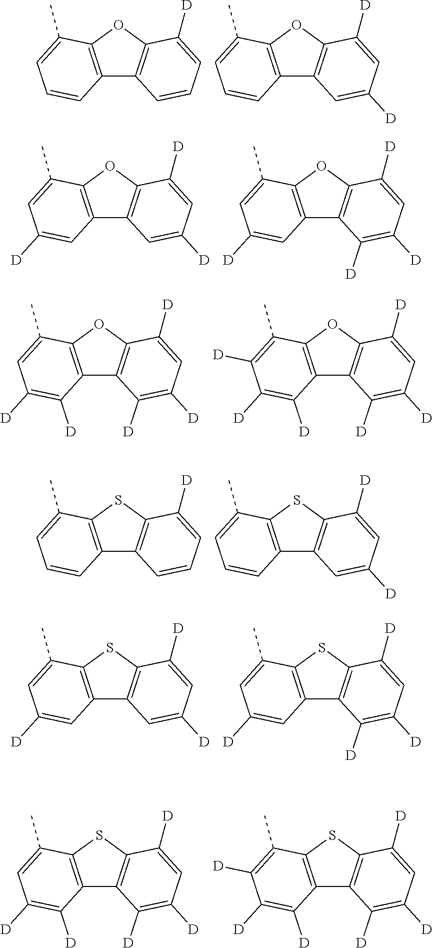

12. The compound according to claim 1, wherein:
the compound is any one of the following Chemical Formulas 1-1 to 1-4: 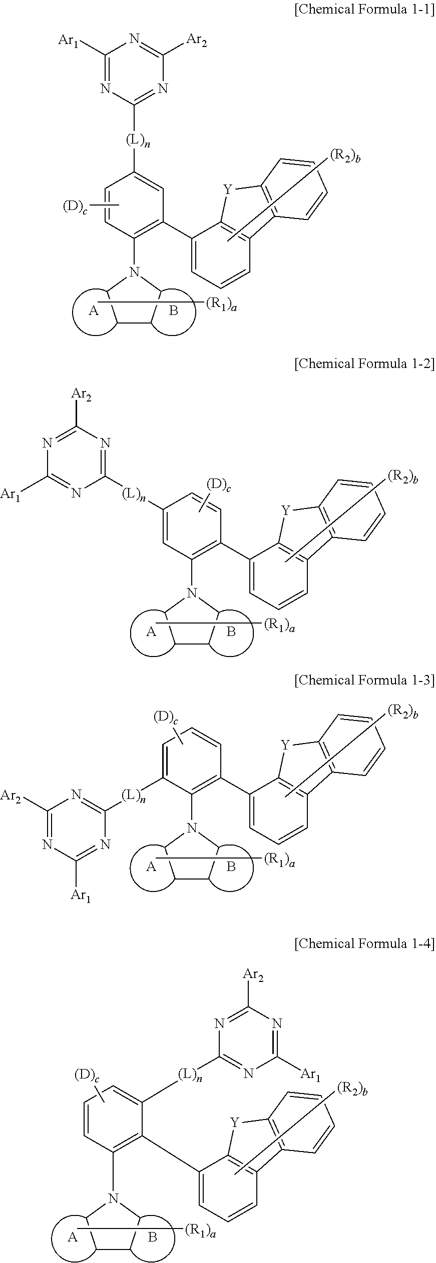
wherein, in Chemical Formulas 1-1 to 1-4;
A and B are each independently a benzene ring, naphthalene ring, phenanthrene ring, or carbazole ring which is fused with an adjacent pentagonal ring, and Y, L, n, Ar₁, Ar₂, R₁, R₂, D, a, b and c are as defined in claim 1.

13. The compound according to claim 1, wherein:
the compound contains 1 to 30 deuteriums.

14. The compound according to claim 1, wherein:
the compound is any one compound selected from the group consisting of the following compounds:

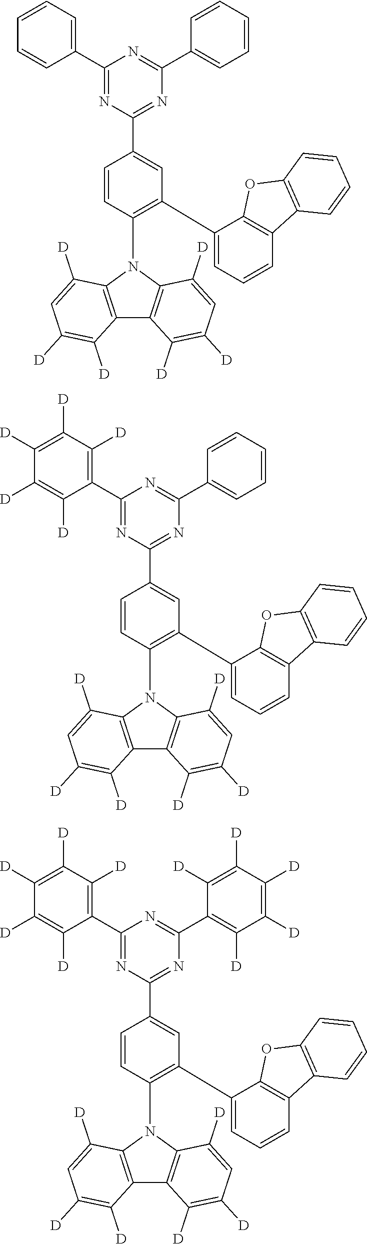

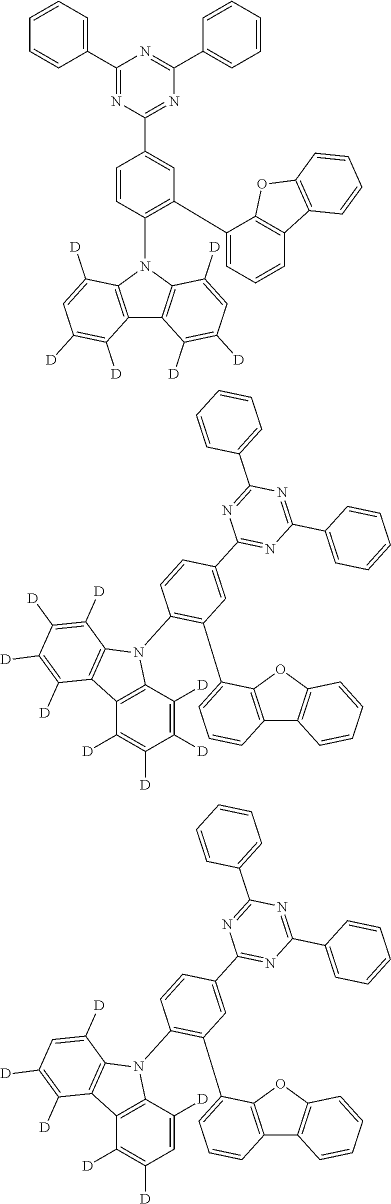

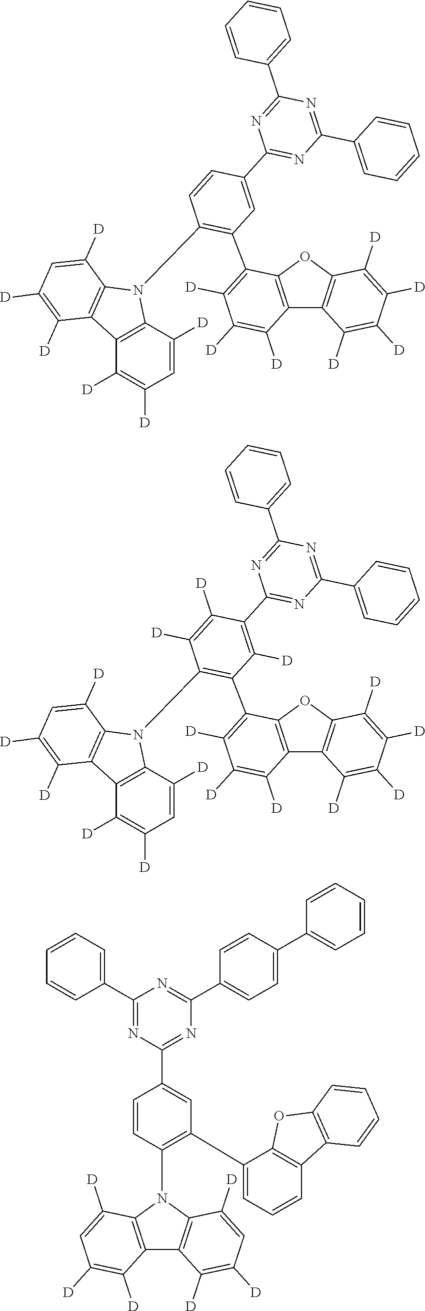 [SMILES: [2H]c1cc([2H])c2c(c1[2H])c1c([2H])c([2H])cc([2H])c1n2-c1ccc(-c2nc(-c3ccccc3)nc(-c3ccccc3)n2)cc1-c1c([2H])c([2H])c([2H])c2c1oc1c([2H])c([2H])c([2H])c([2H])c12 ; [2H]c1cc([2H])c2c(c1[2H])c1c([2H])c([2H])cc([2H])c1n2-c1c([2H])c([2H])c(-c2nc(-c3ccccc3)nc(-c3ccccc3)n2)c([2H])c1-c1c([2H])c([2H])c([2H])c2c1oc1c([2H])c([2H])c([2H])c([2H])c12 ; [2H]c1cc([2H])c2c(c1[2H])c1c([2H])c([2H])cc([2H])c1n2-c1ccc(-c2nc(-c3ccccc3)nc(-c3ccc(-c4ccccc4)cc3)n2)cc1-c1cccc2c1oc1ccccc12]

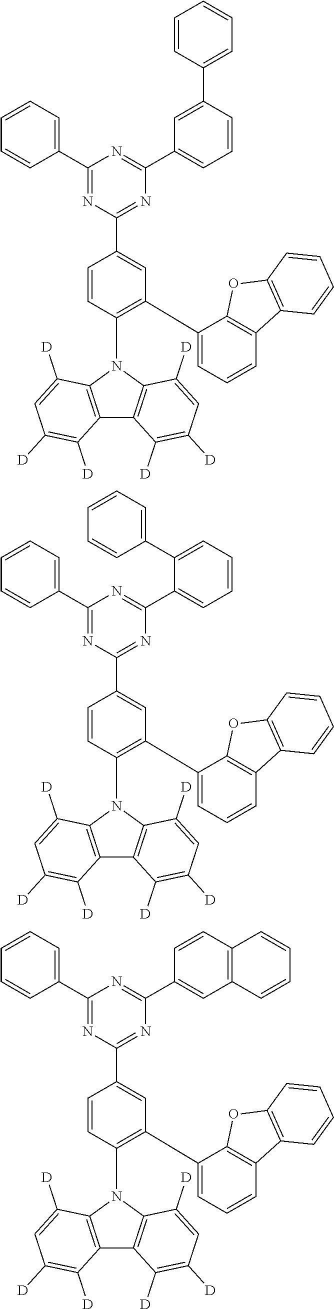

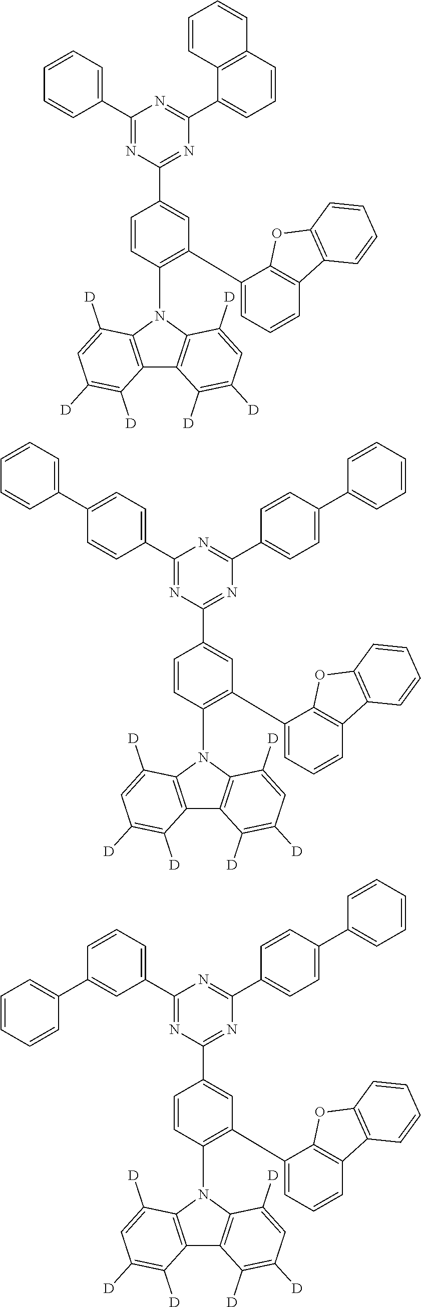 [SMILES: [2H]c1cc([2H])c2c(c1[2H])c1c([2H])c([2H])cc([2H])c1n2-c1ccc(-c2nc(-c3ccccc3)nc(-c3cccc4ccccc34)n2)cc1-c1cccc2c1oc1ccccc12 ; [2H]c1cc([2H])c2c(c1[2H])c1c([2H])c([2H])cc([2H])c1n2-c1ccc(-c2nc(-c3ccc(-c4ccccc4)cc3)nc(-c3ccc(-c4ccccc4)cc3)n2)cc1-c1cccc2c1oc1ccccc12 ; [2H]c1cc([2H])c2c(c1[2H])c1c([2H])c([2H])cc([2H])c1n2-c1ccc(-c2nc(-c3ccc(-c4ccccc4)cc3)nc(-c3cccc(-c4ccccc4)c3)n2)cc1-c1cccc2c1oc1ccccc12]

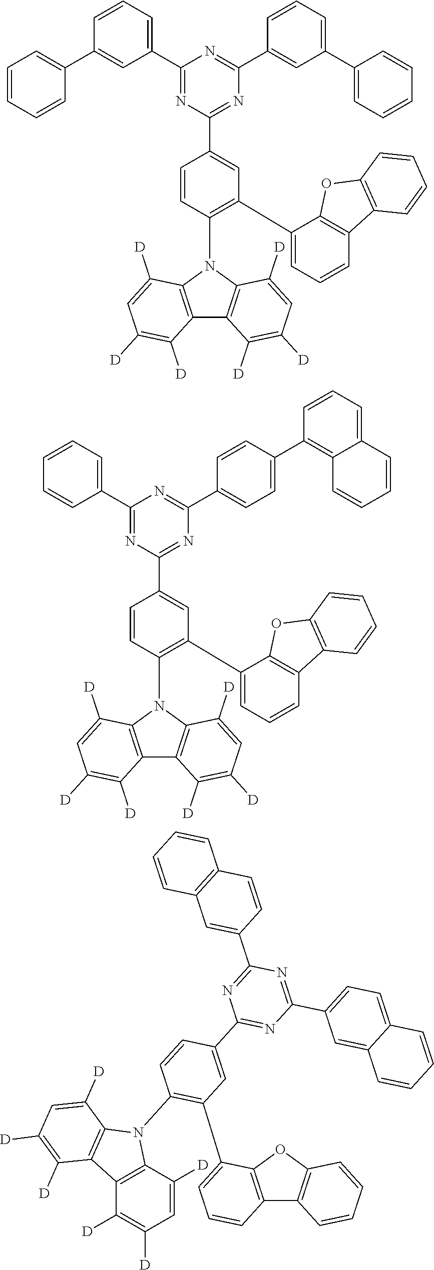

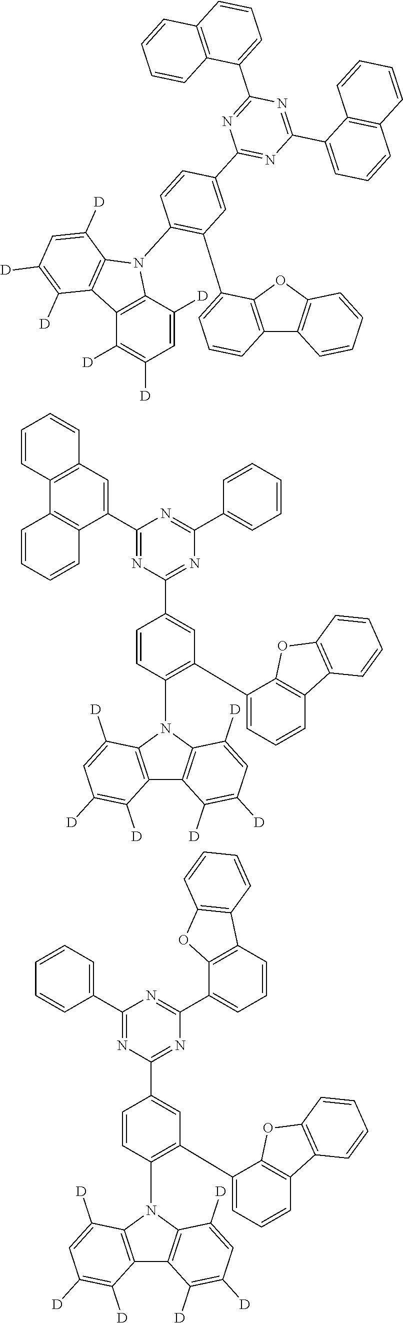

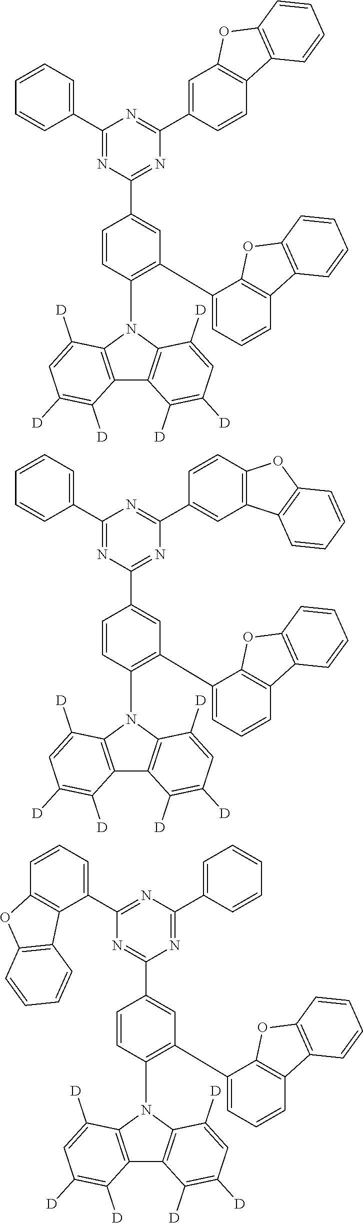

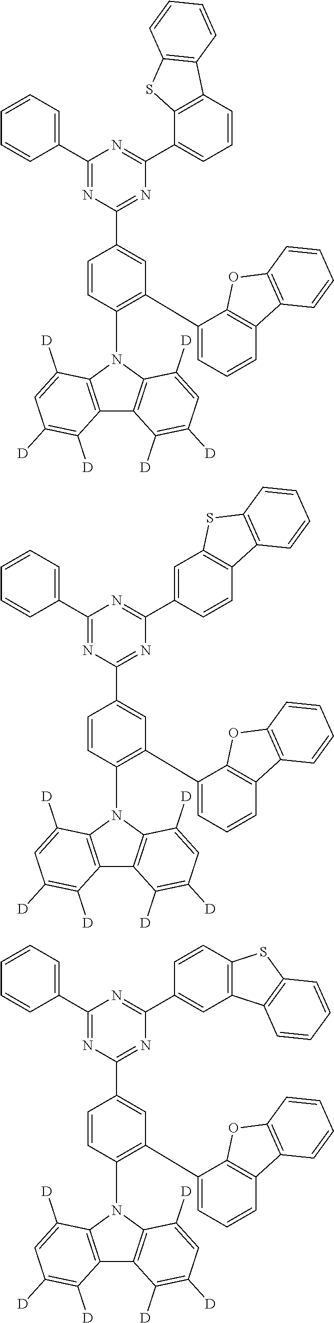

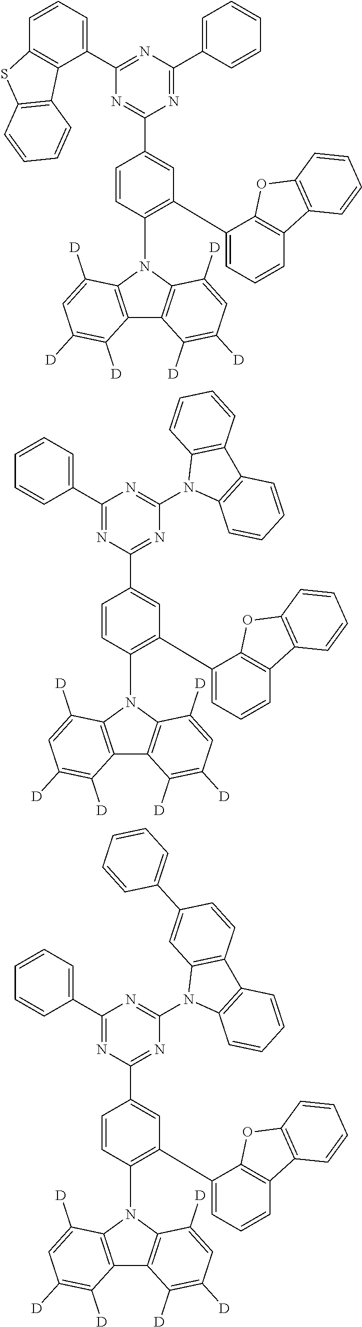

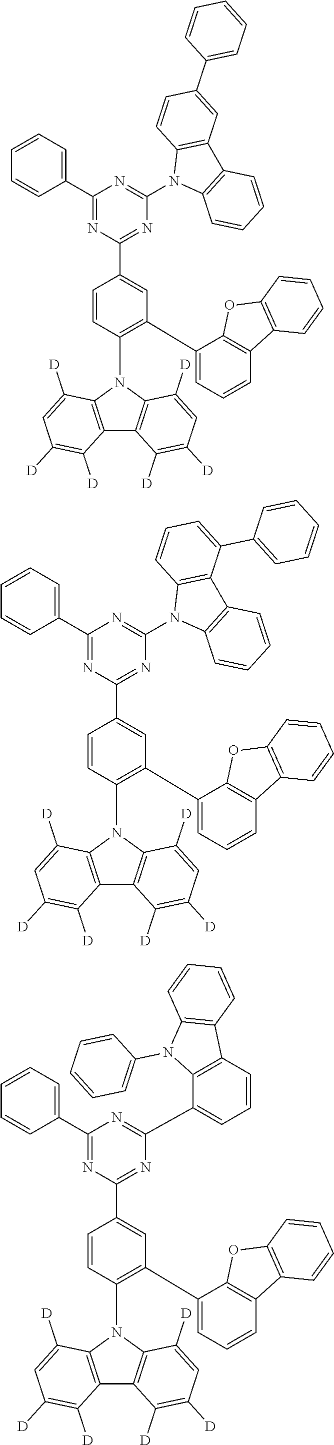

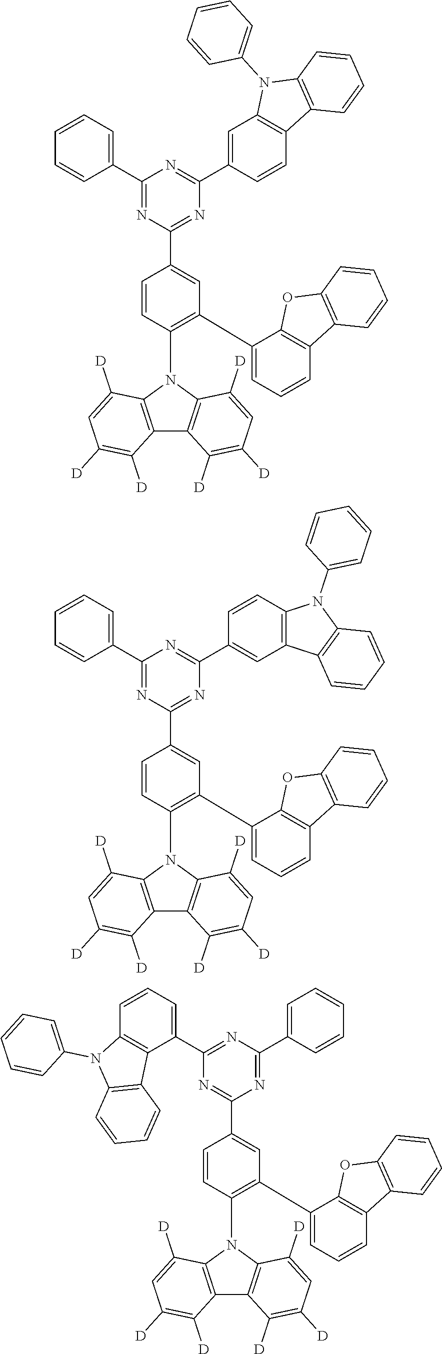

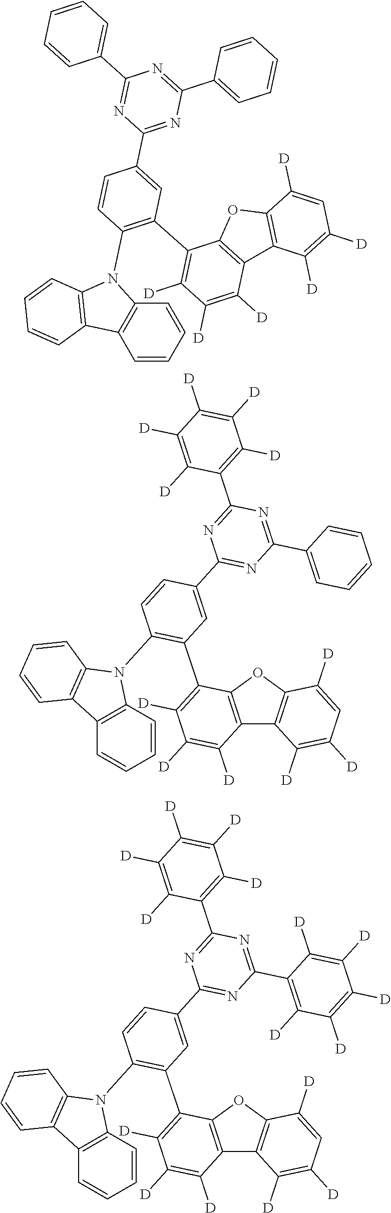

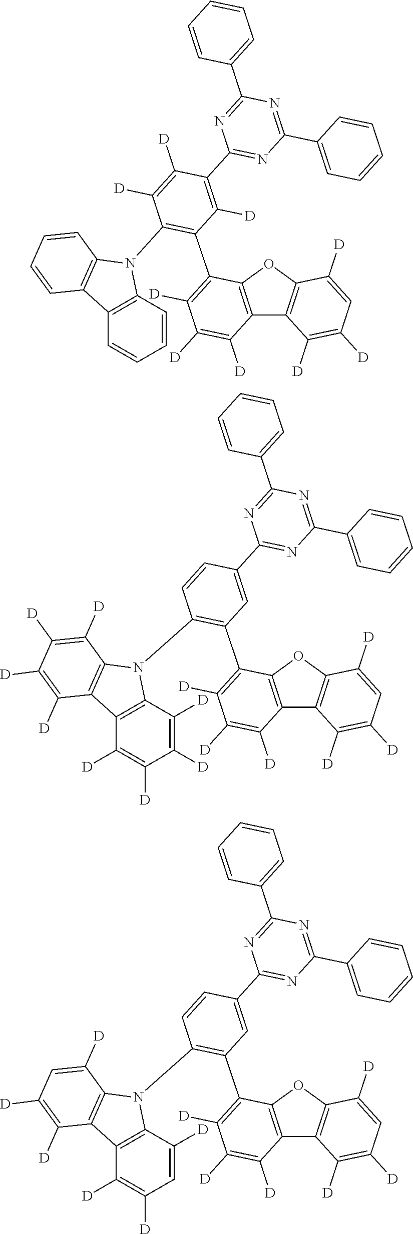

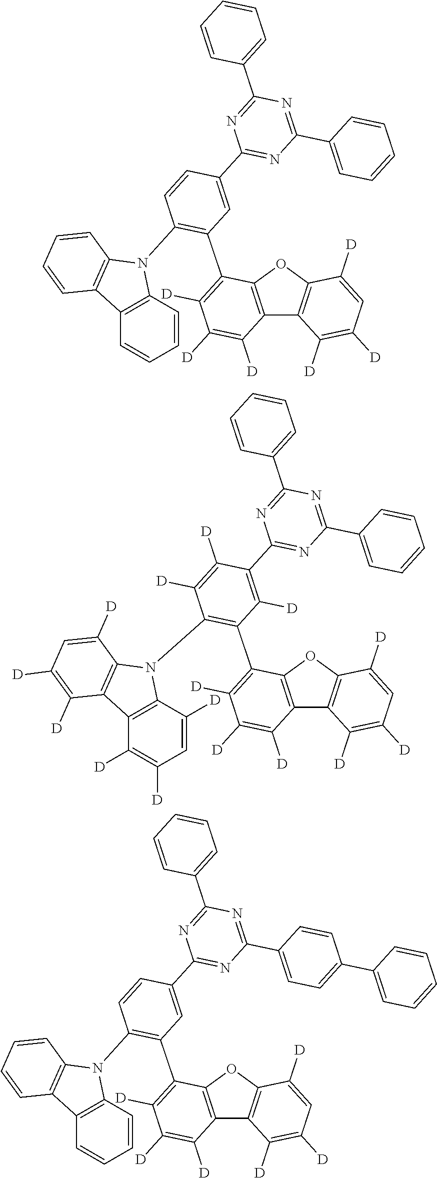

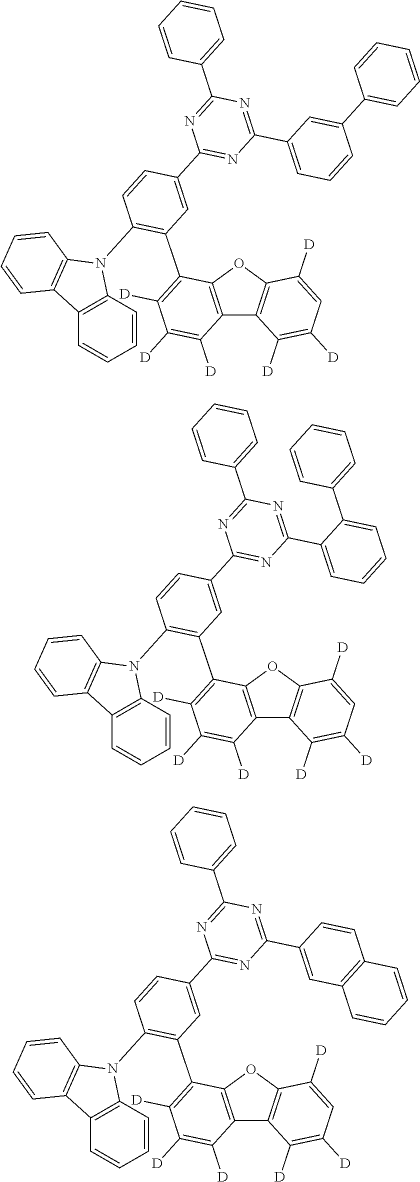

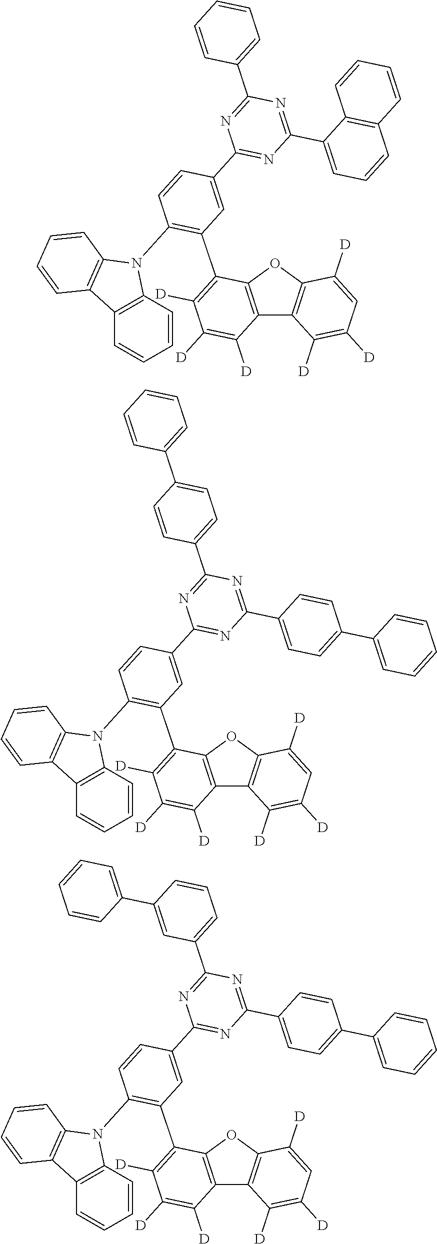

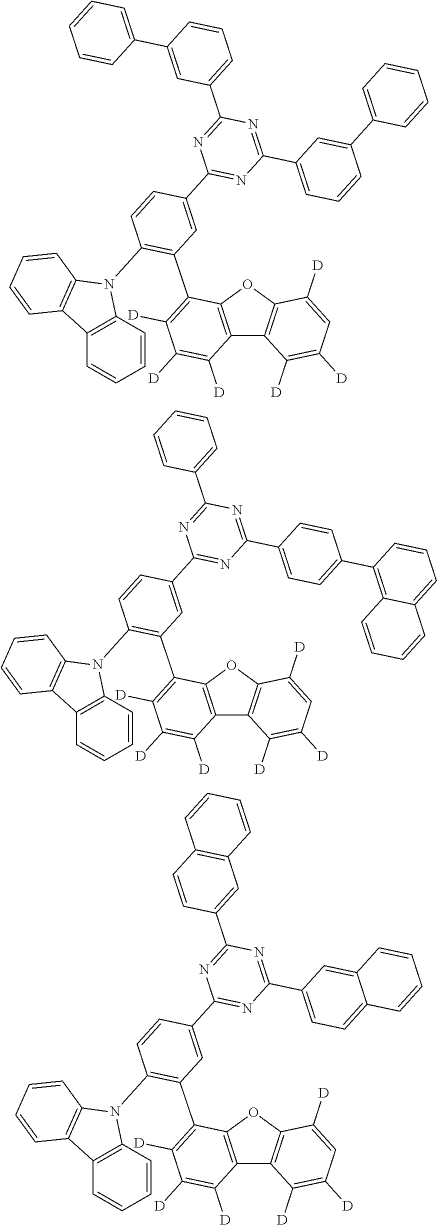

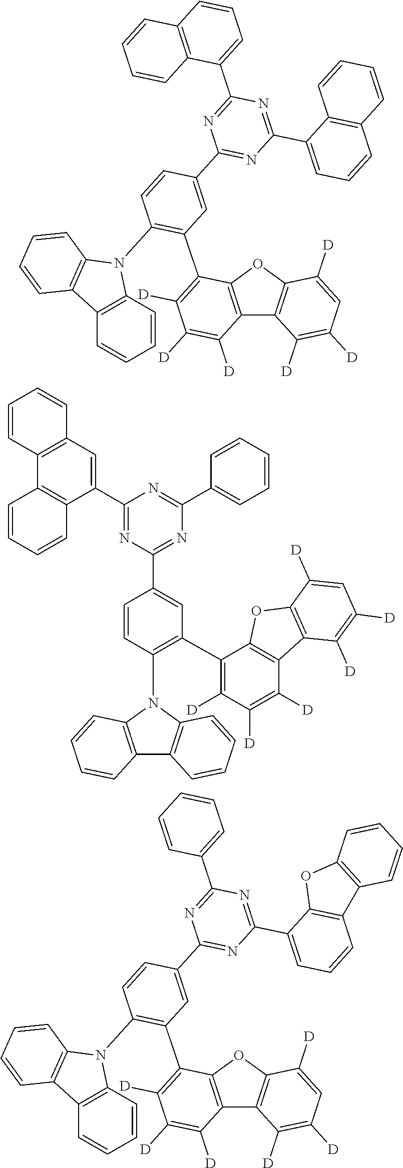

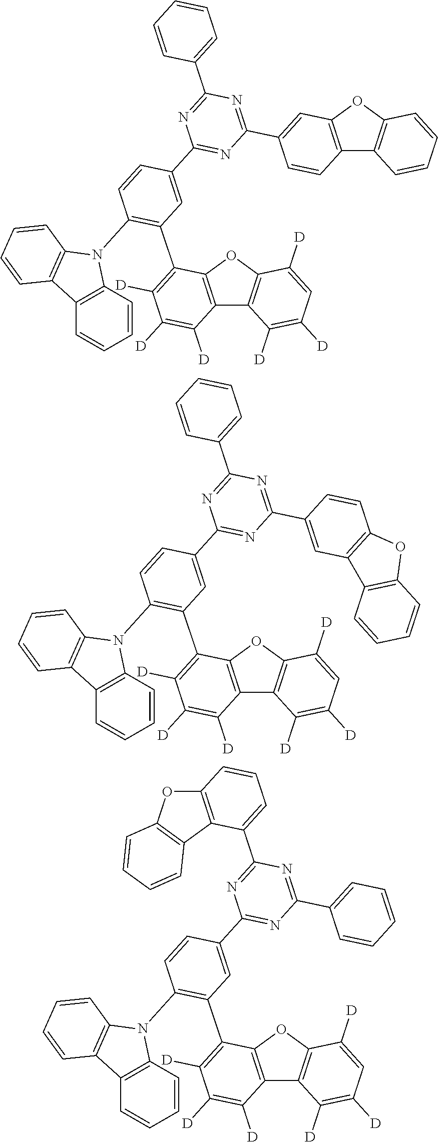

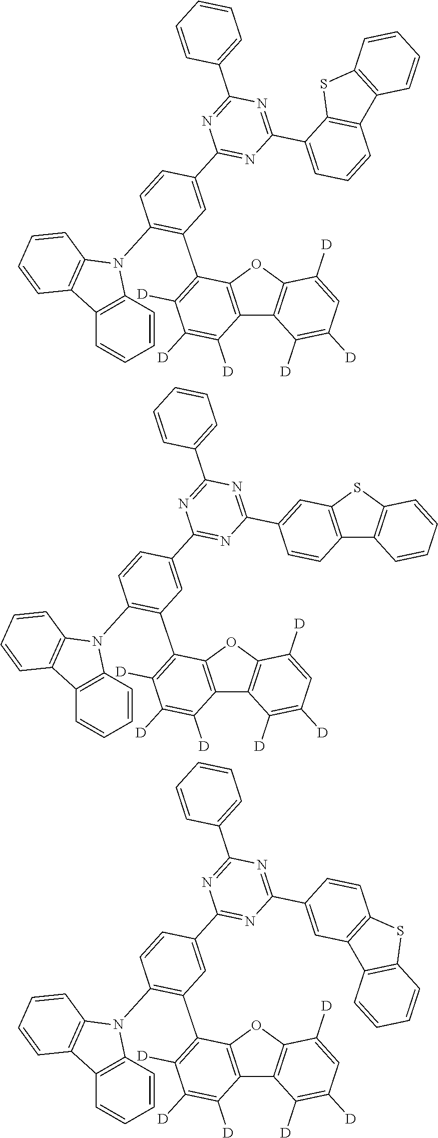

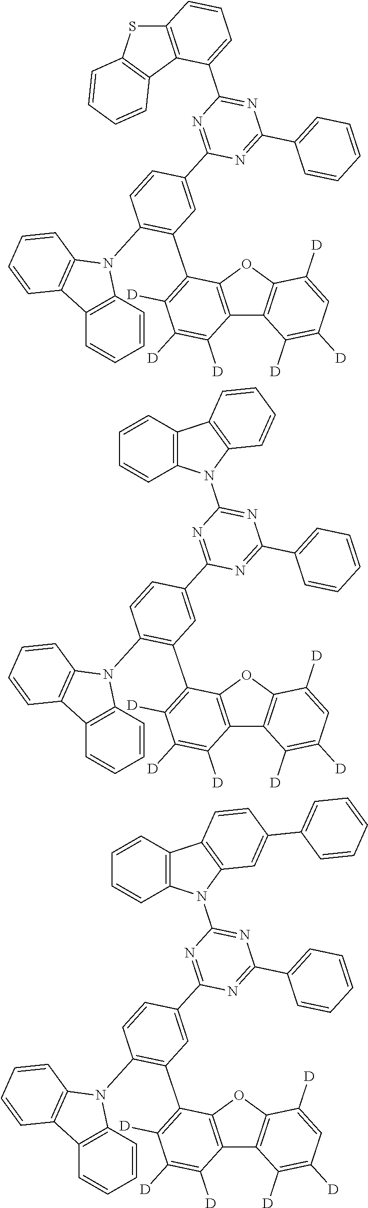

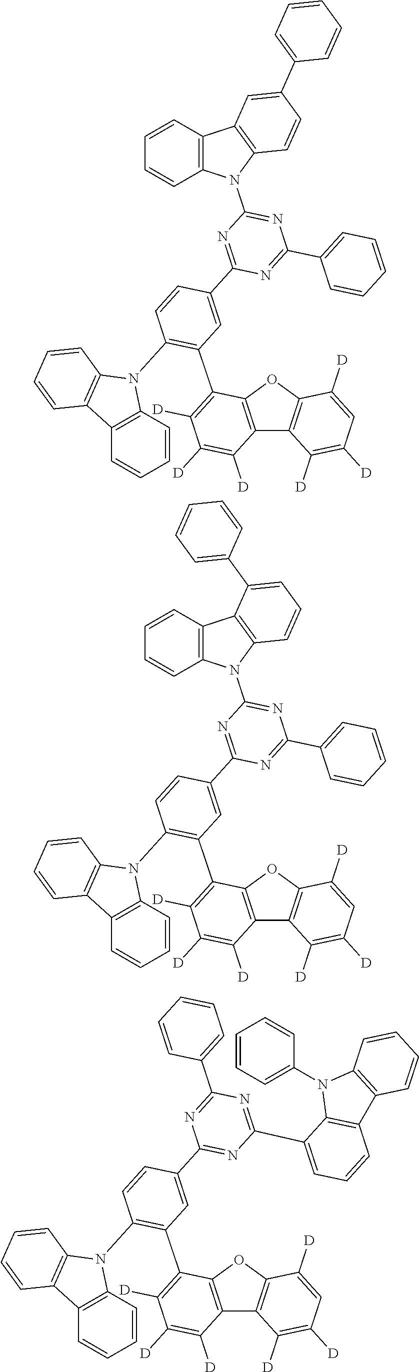

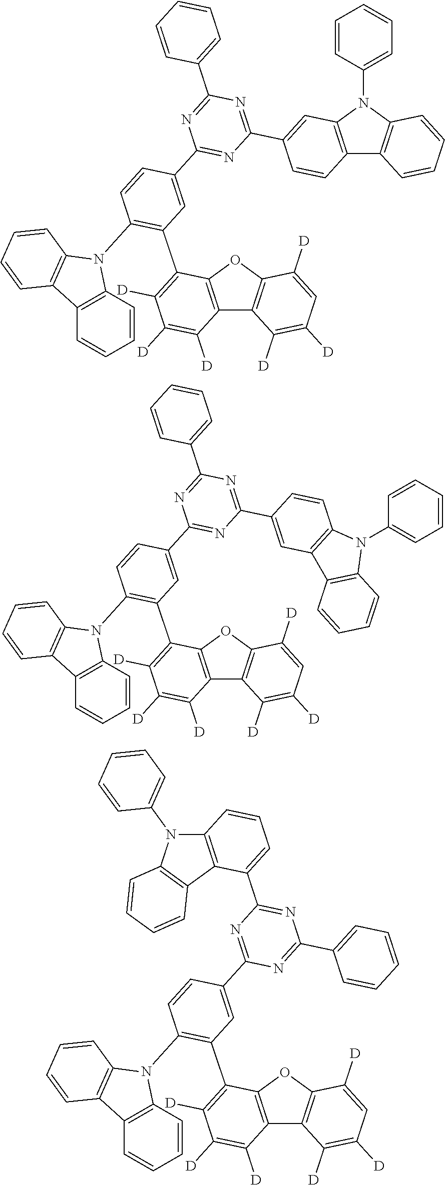

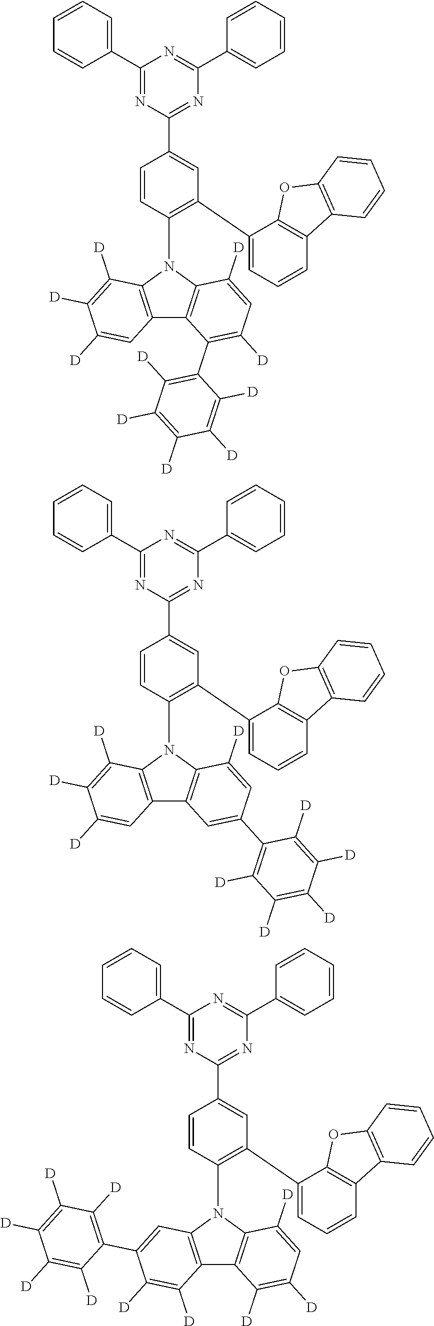

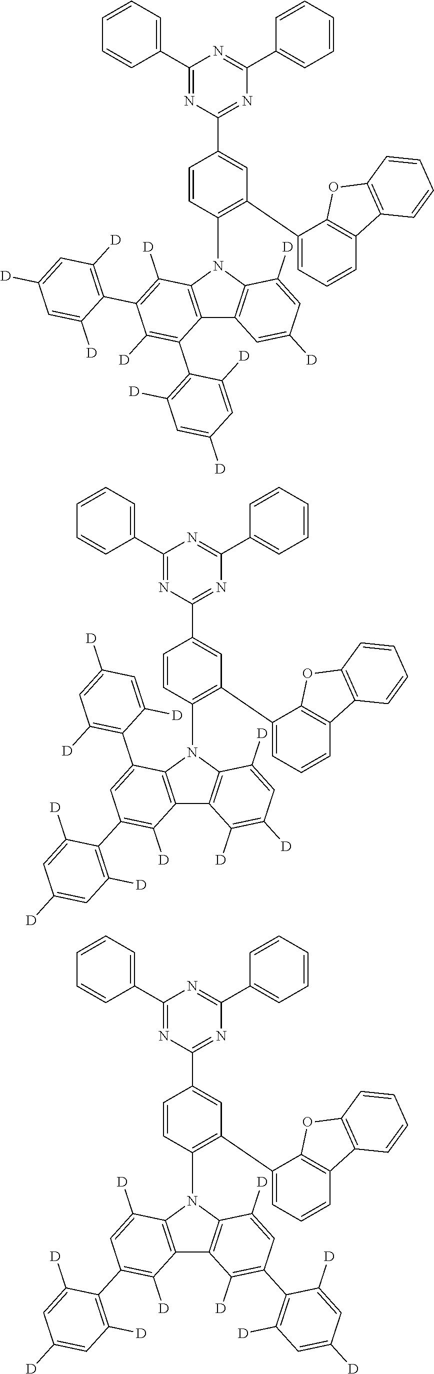

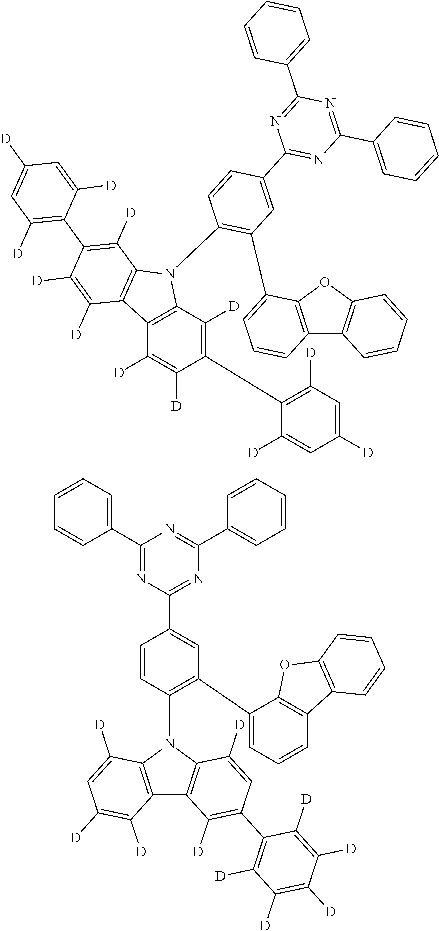

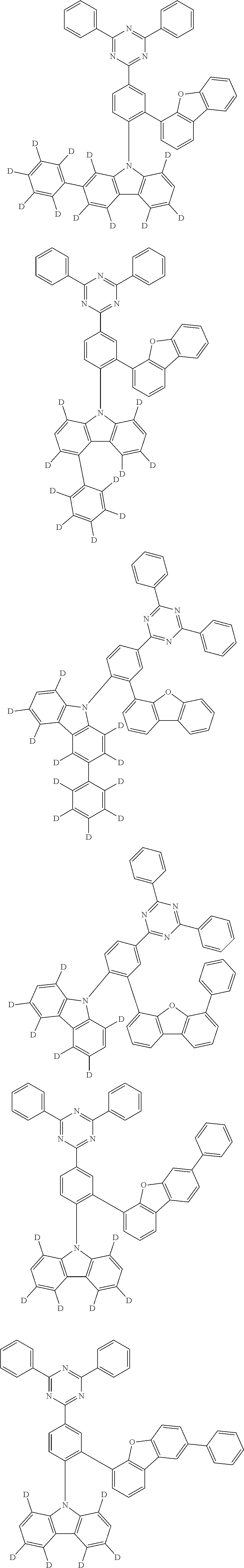

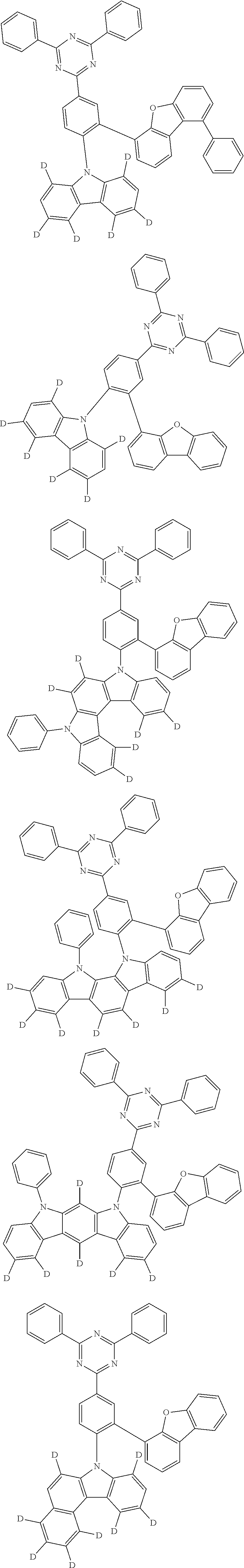

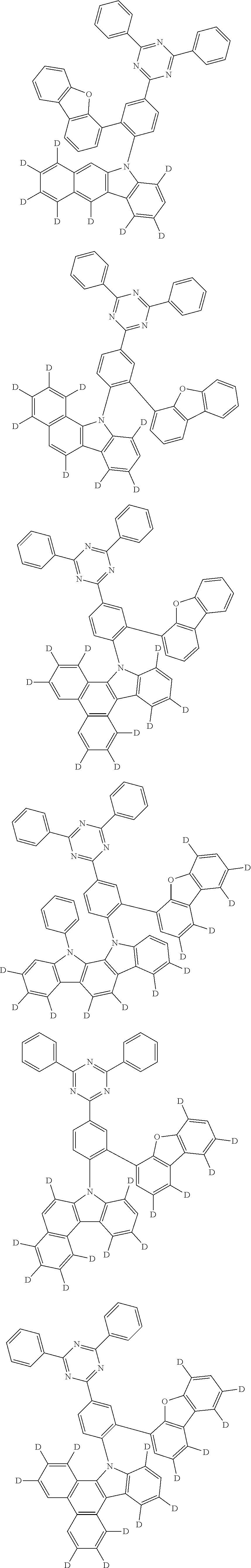

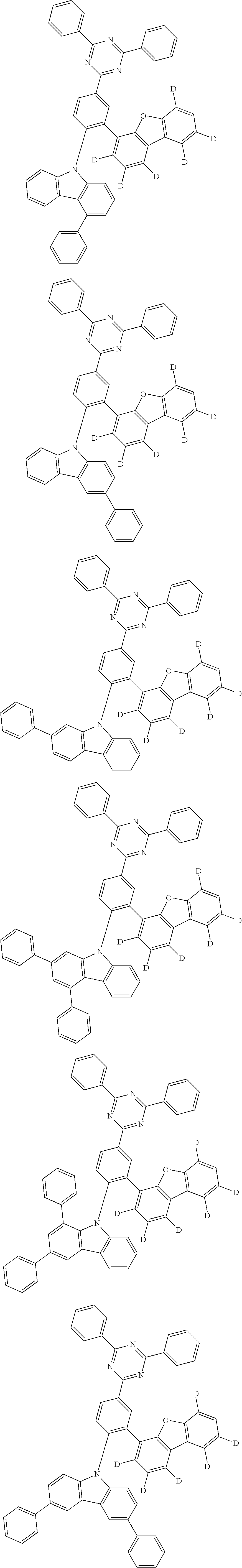

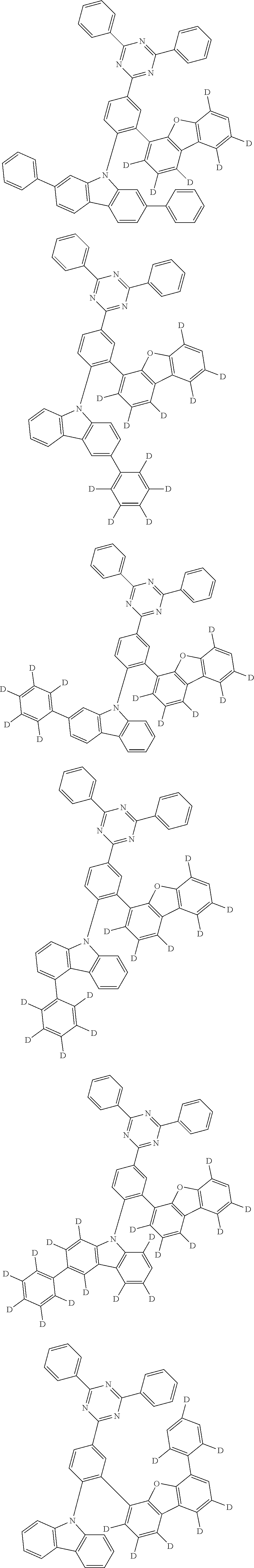

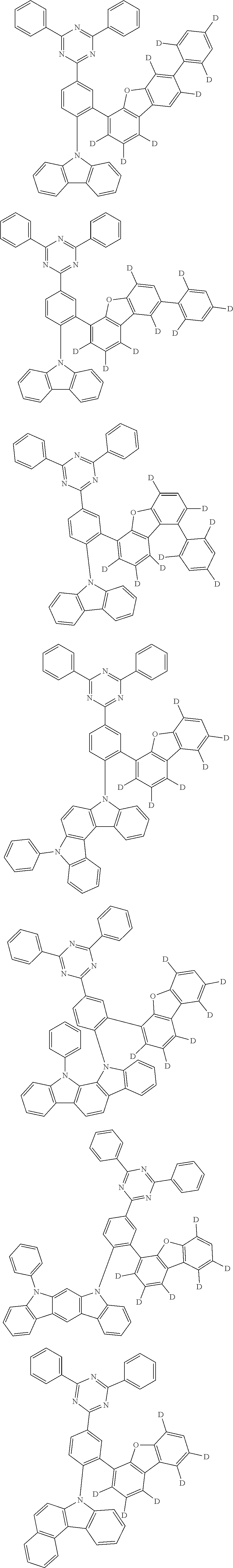

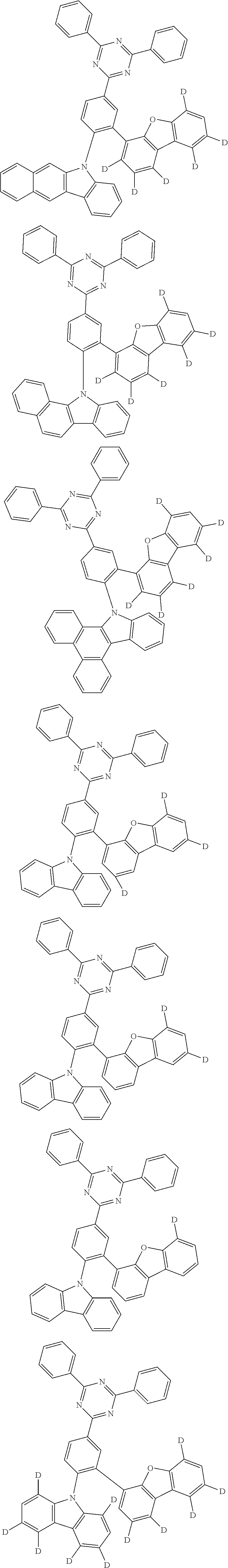

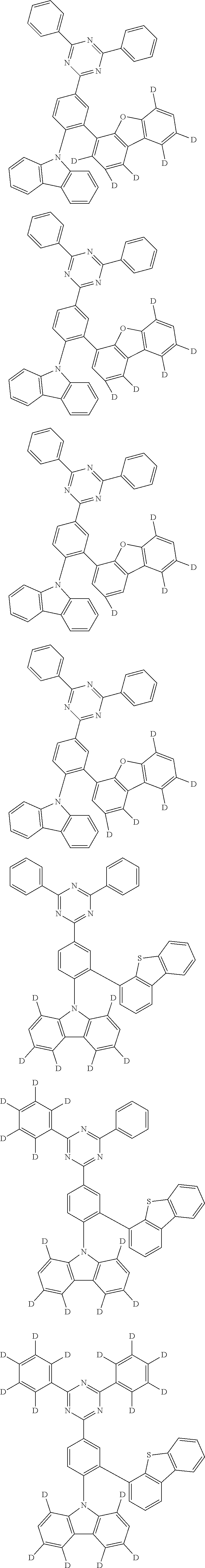

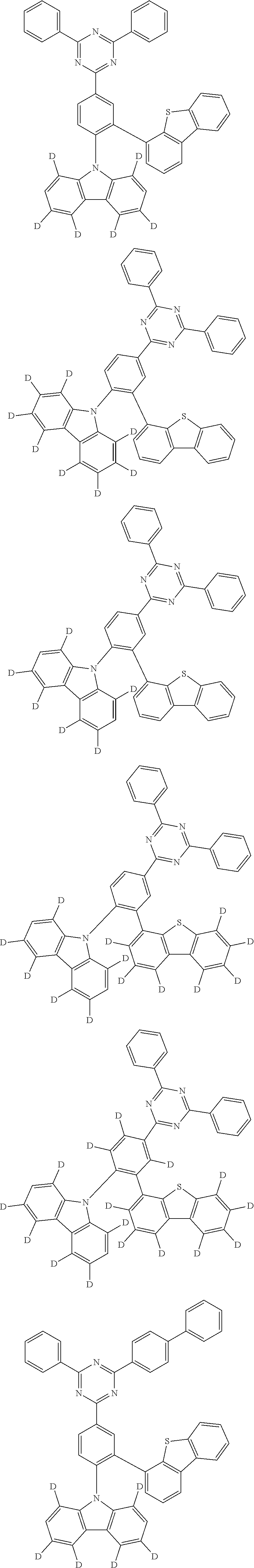

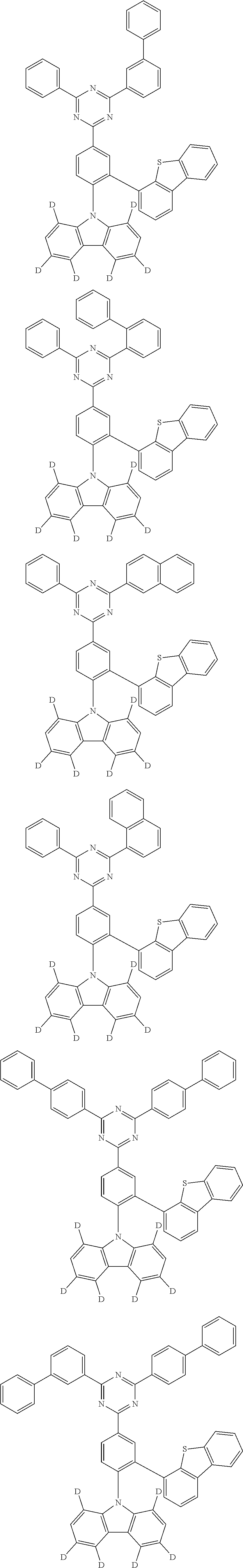

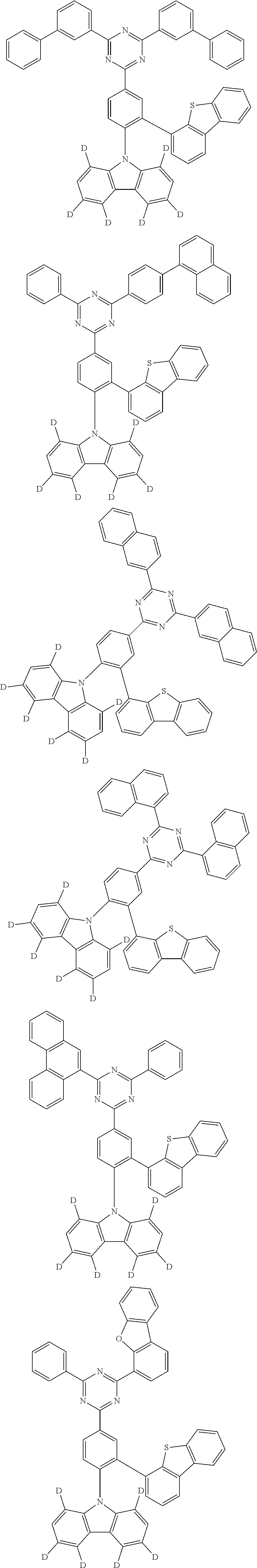

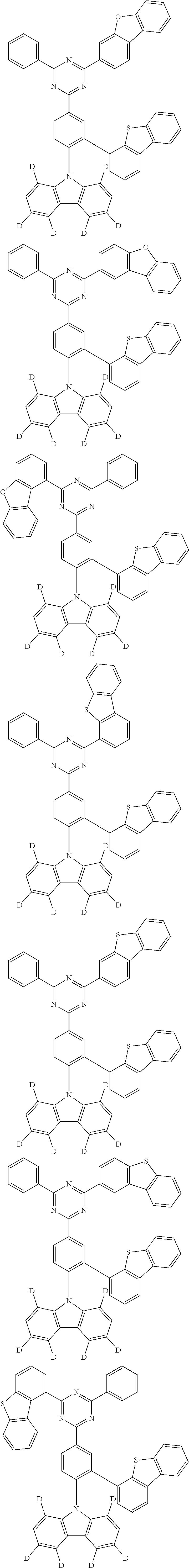

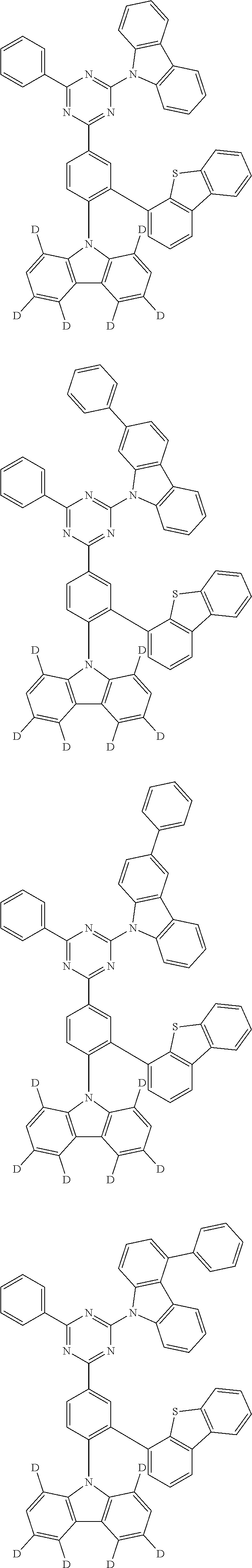

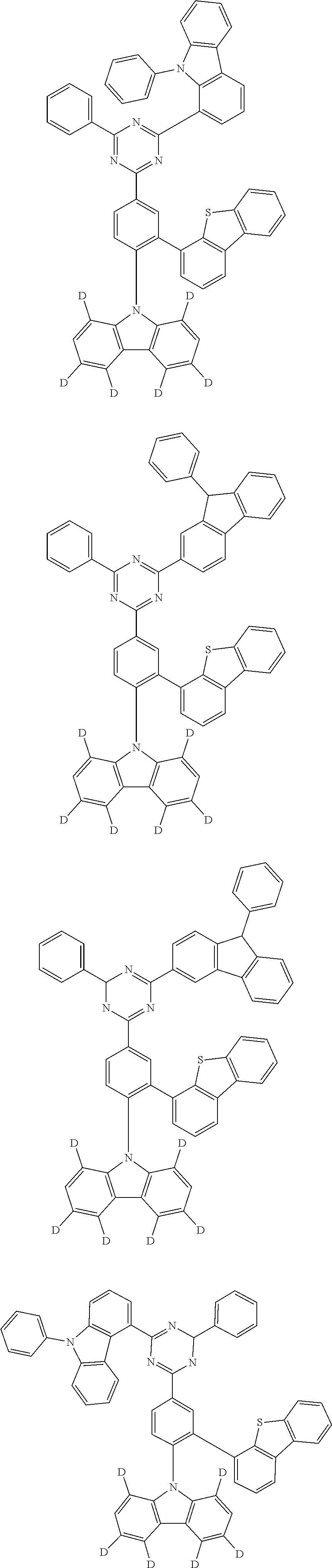

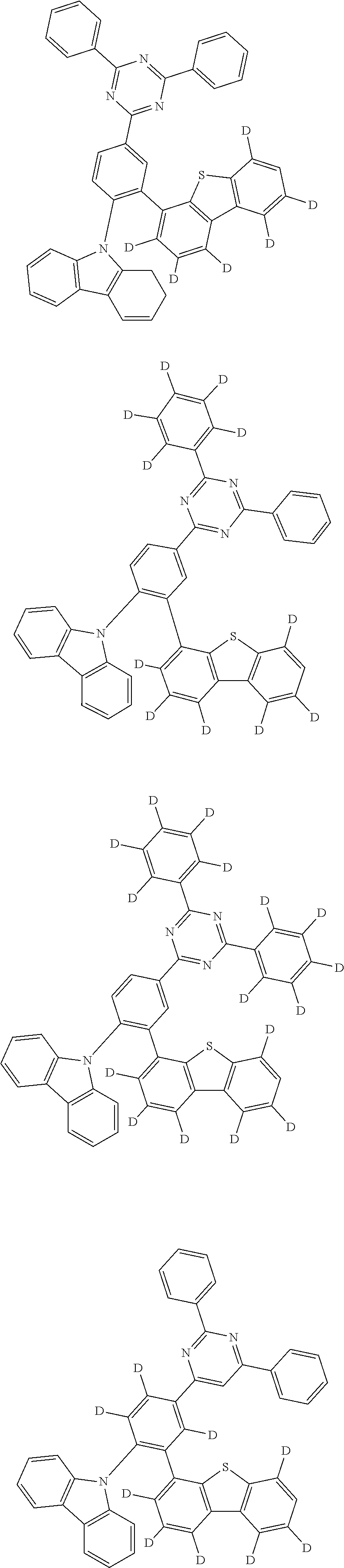

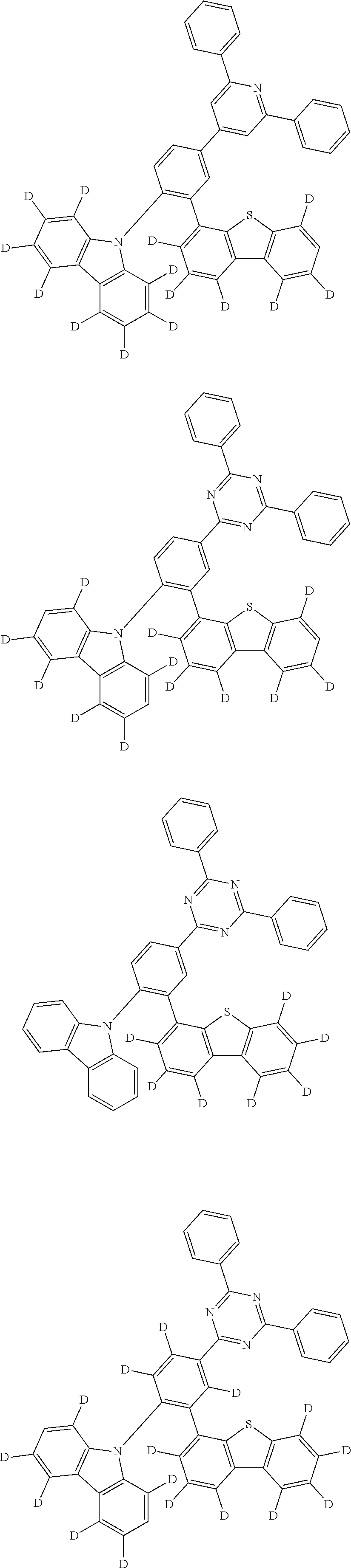

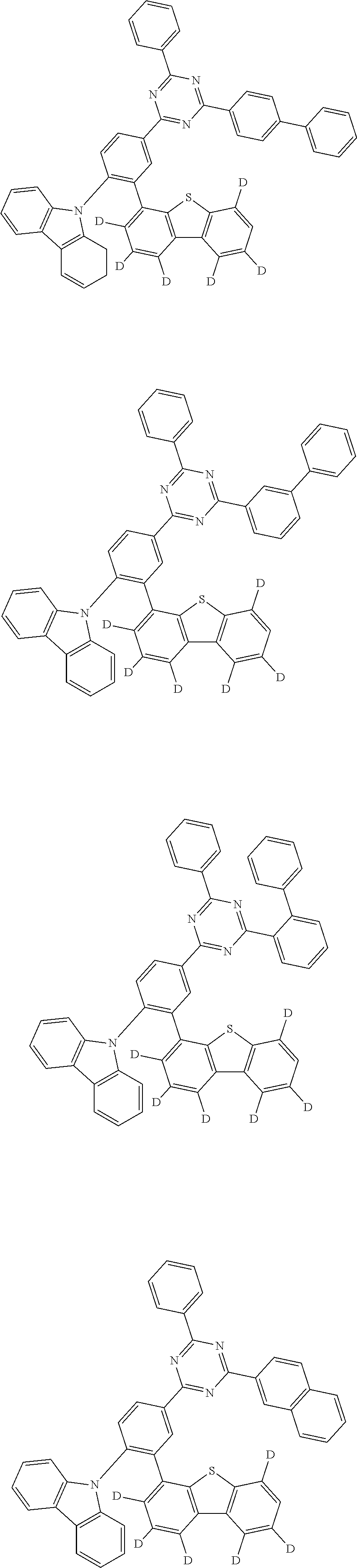

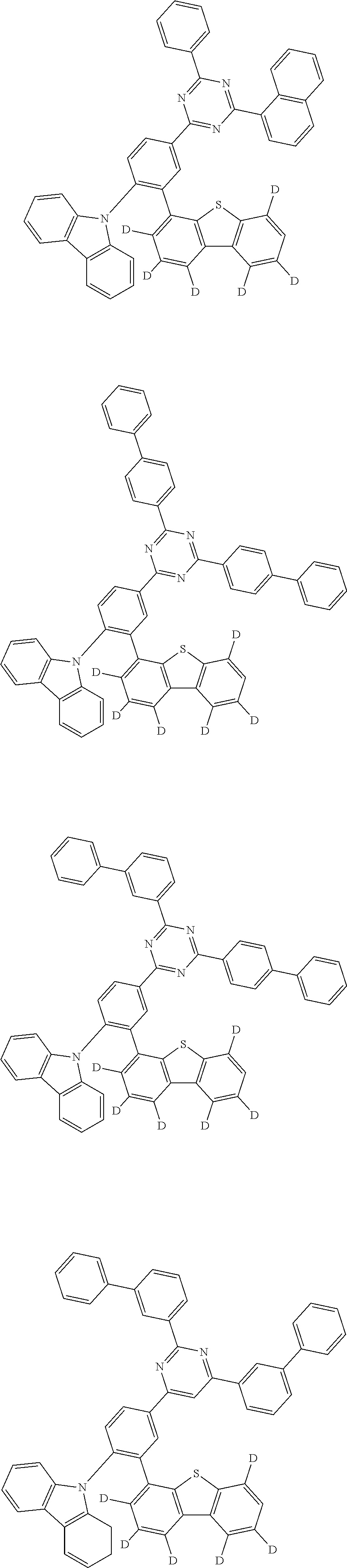

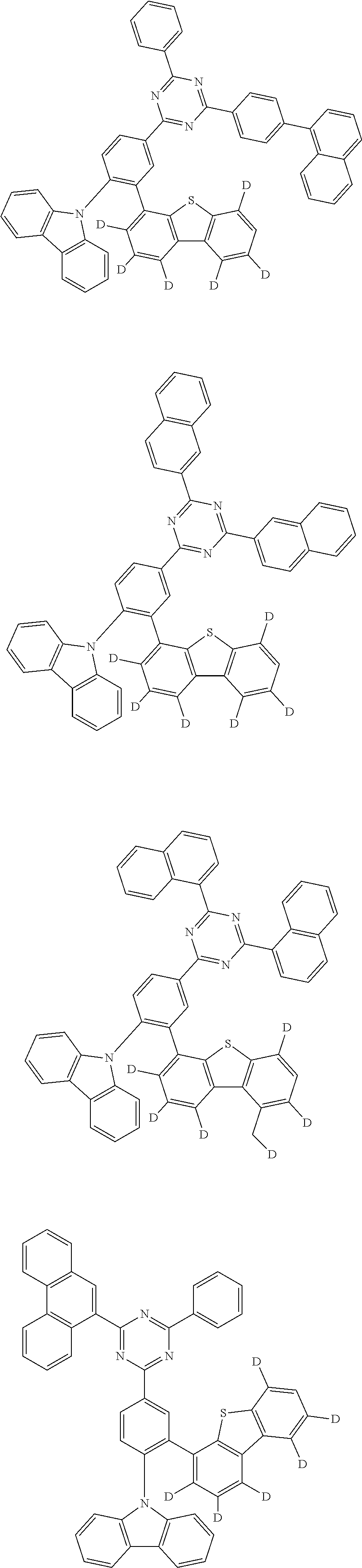

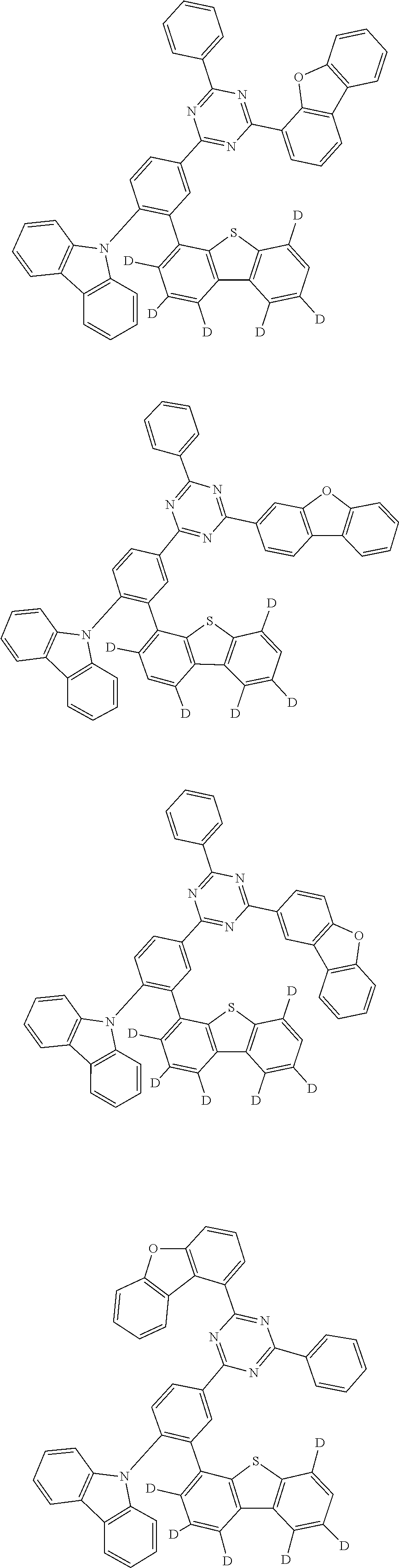

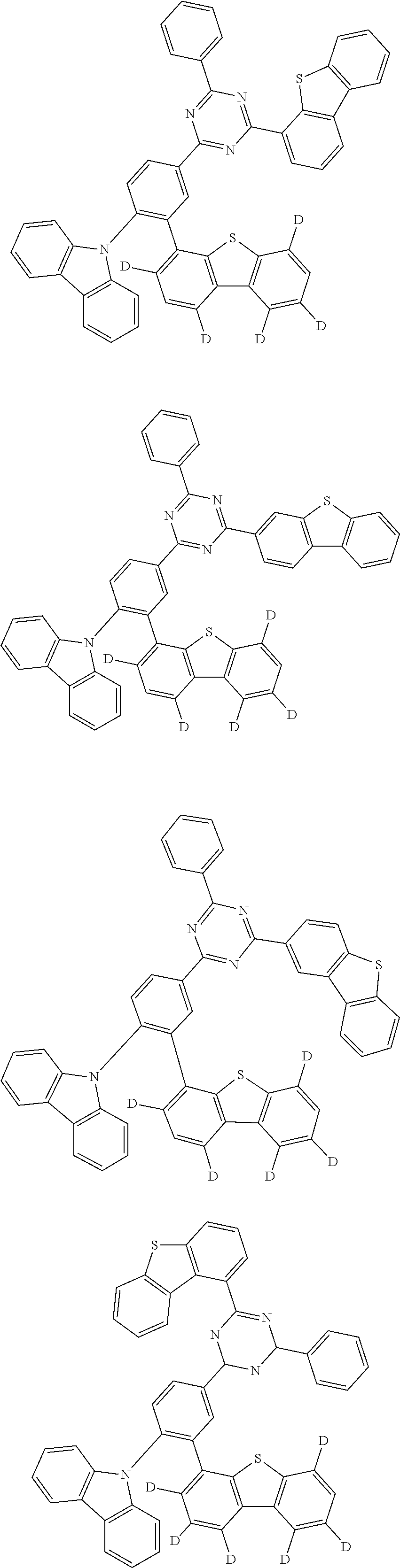

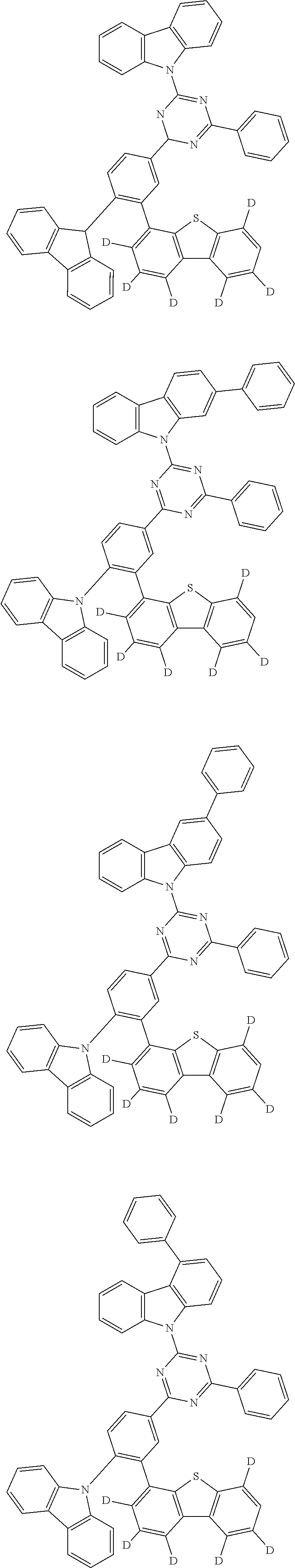

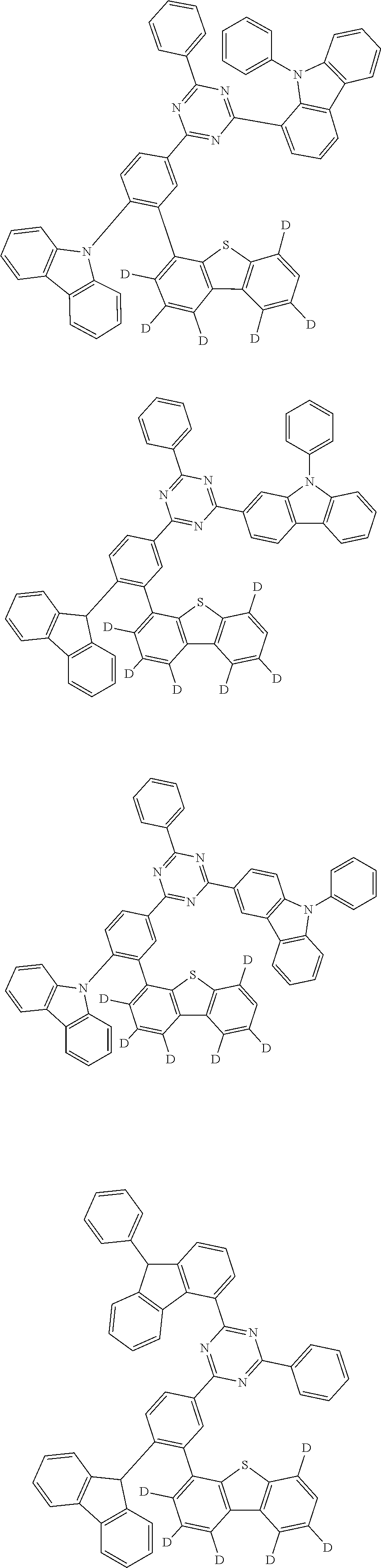

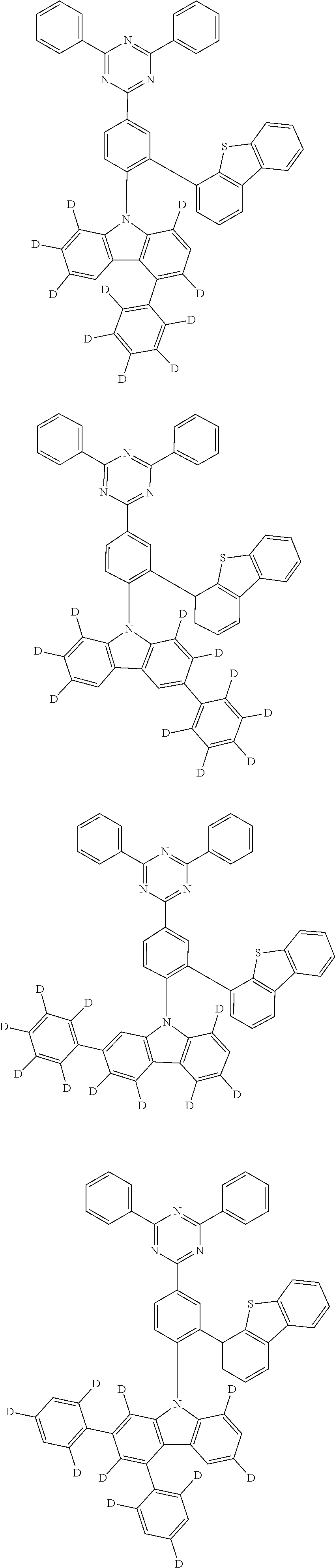

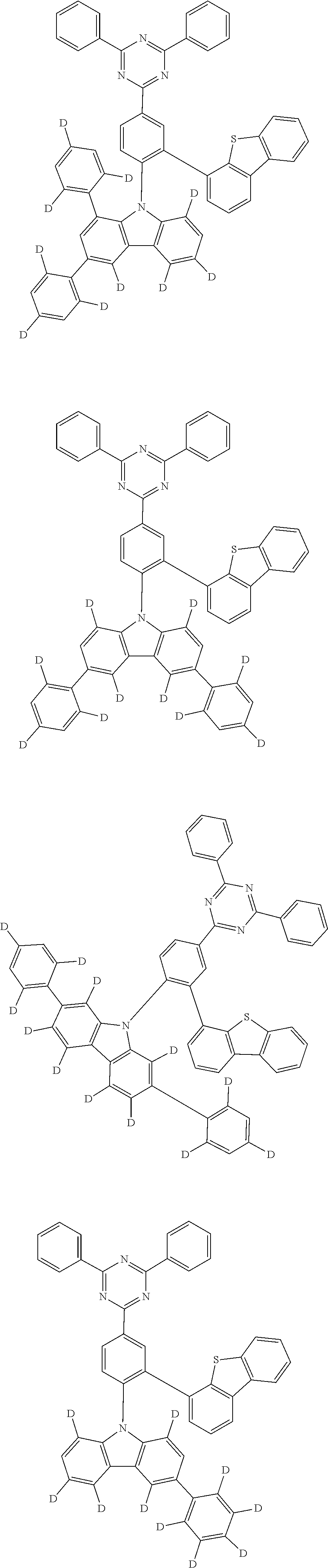

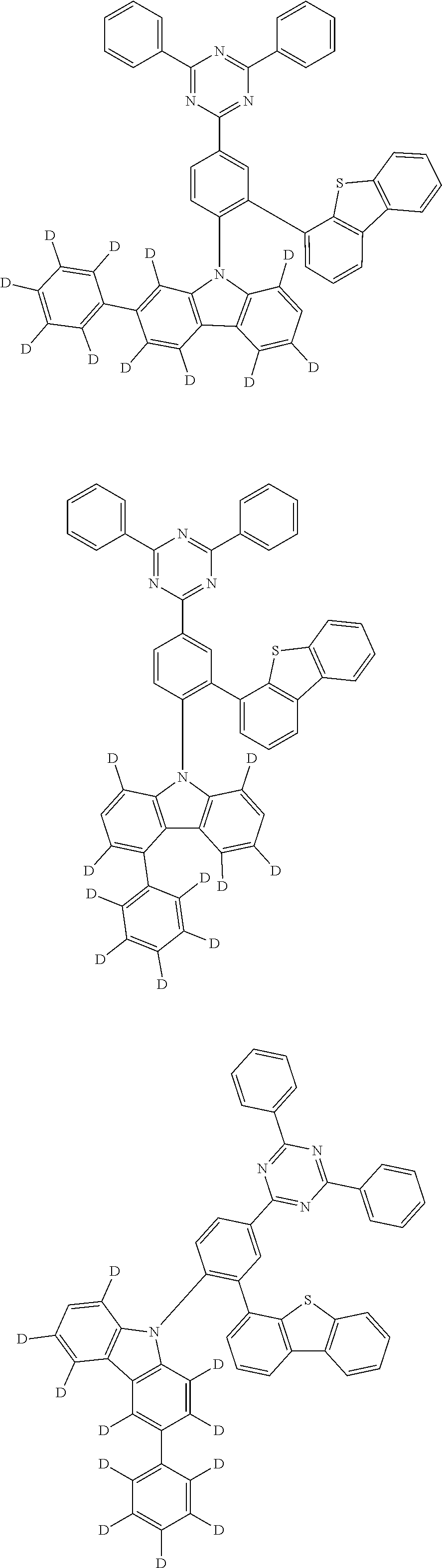 [SMILES: [2H]c1cc([2H])c2c(c1[2H])c1c([2H])c([2H])c(-c3c([2H])c([2H])c([2H])c([2H])c3[2H])c([2H])c1n2-c1ccc(-c2nc(-c3ccccc3)nc(-c3ccccc3)n2)cc1-c1cccc2c1sc1ccccc12 ; [2H]c1cc([2H])c2c(c1[2H])c1c(-c3c([2H])c([2H])c([2H])c([2H])c3[2H])c([2H])cc([2H])c1n2-c1ccc(-c2nc(-c3ccccc3)nc(-c3ccccc3)n2)cc1-c1cccc2c1sc1ccccc12 ; [2H]c1cc([2H])c2c(c1[2H])c1c([2H])c(-c3c([2H])c([2H])c([2H])c([2H])c3[2H])c([2H])c([2H])c1n2-c1ccc(-c2nc(-c3ccccc3)nc(-c3ccccc3)n2)cc1-c1cccc2c1sc1ccccc12]

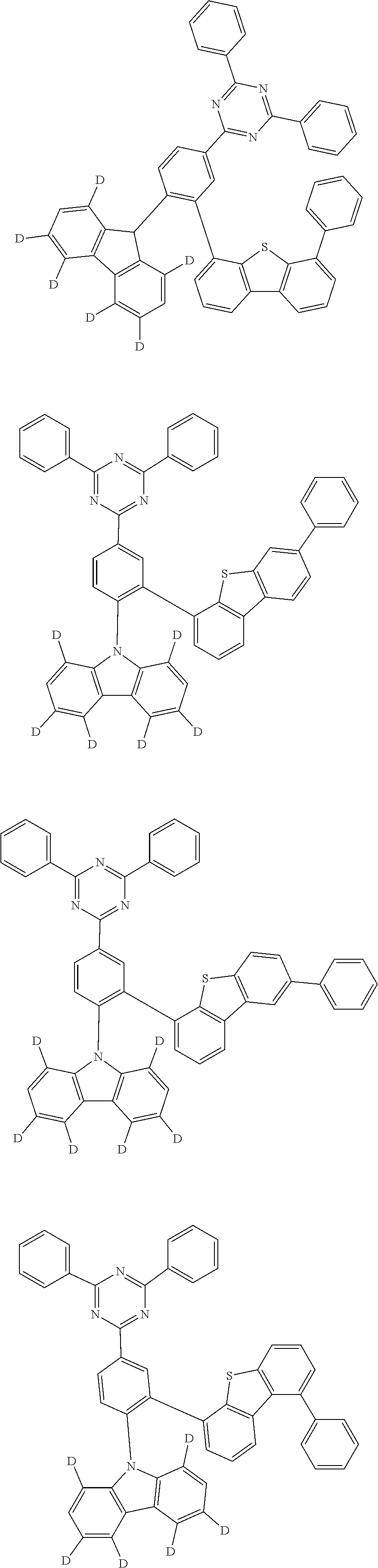

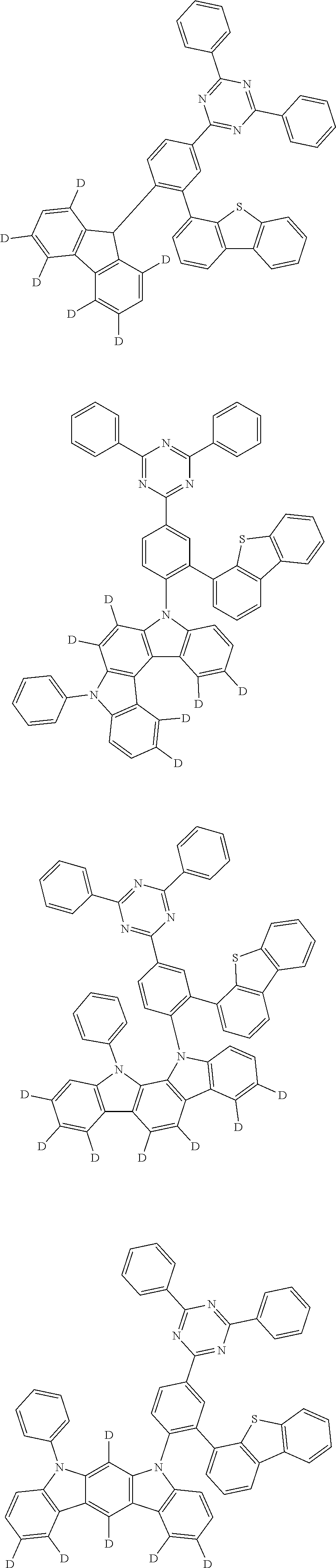

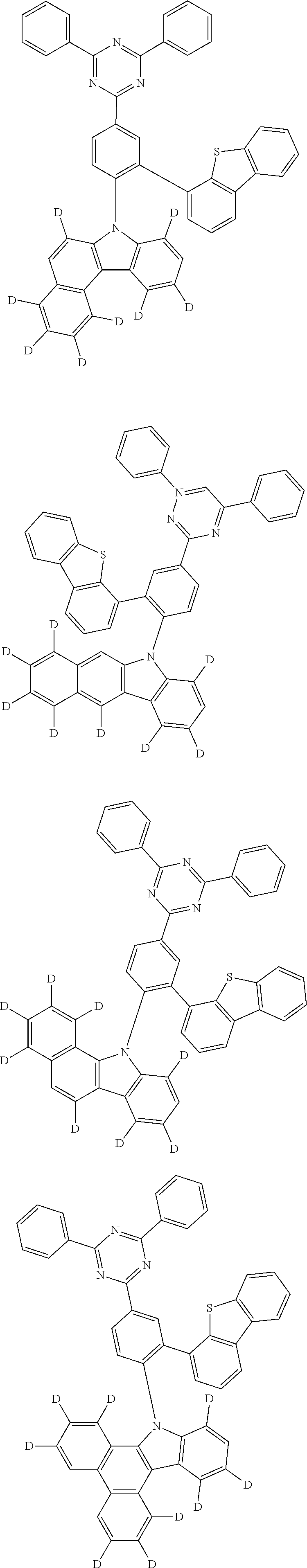

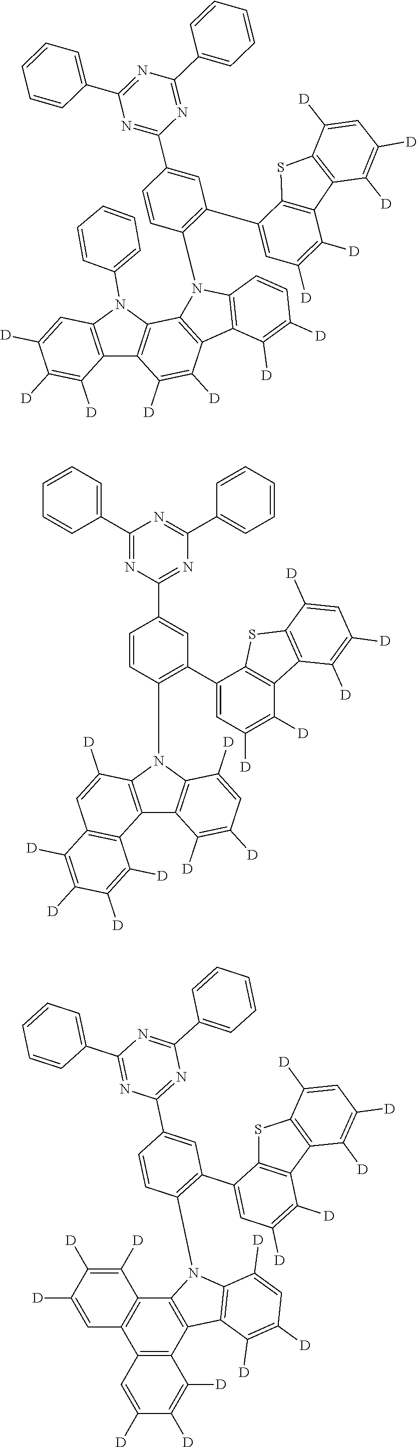

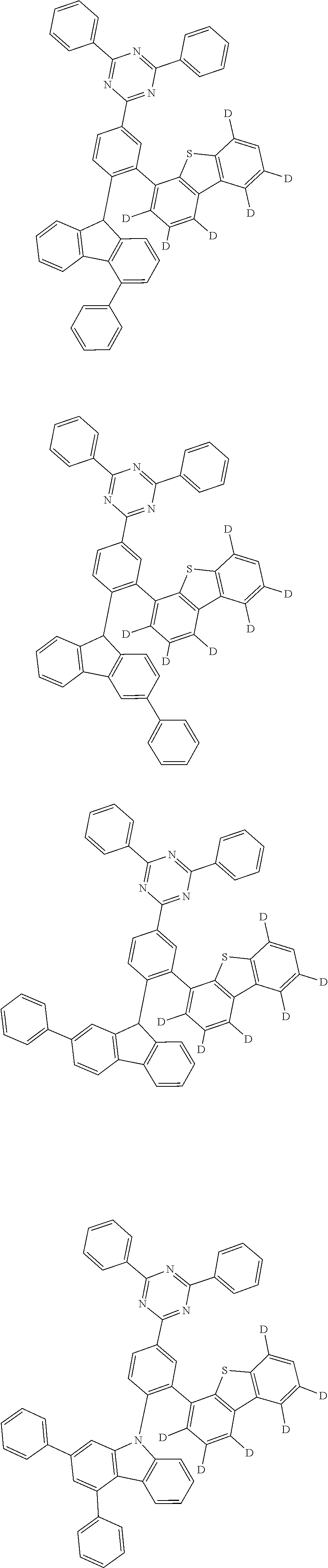

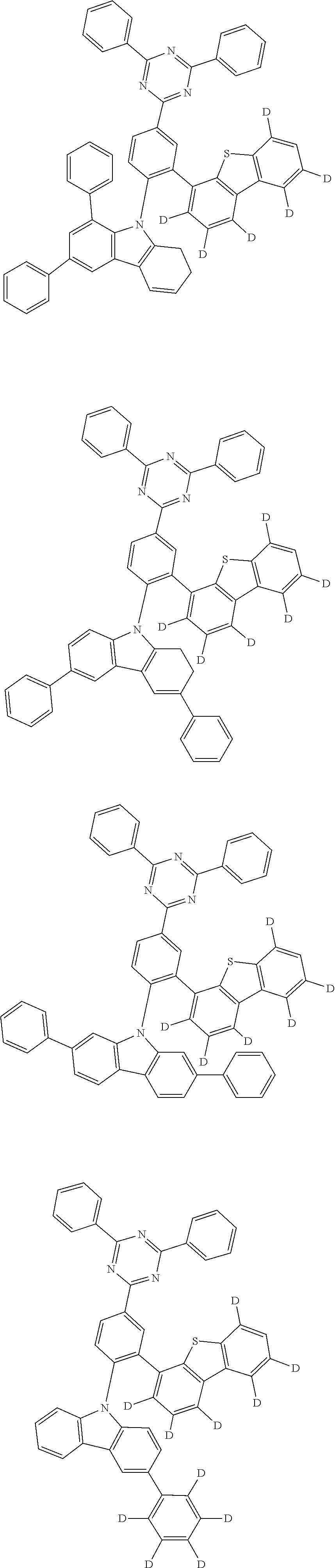

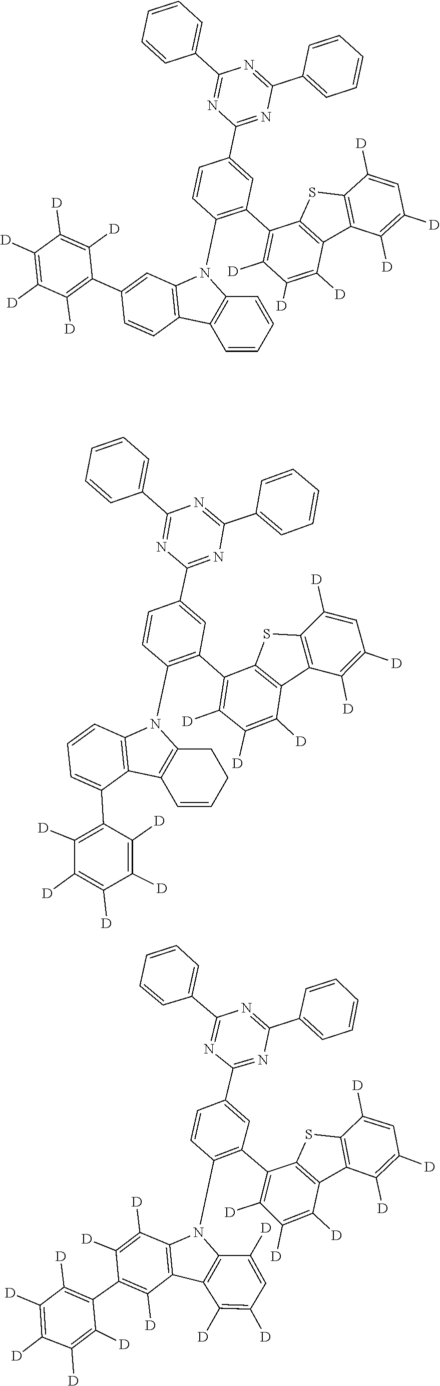

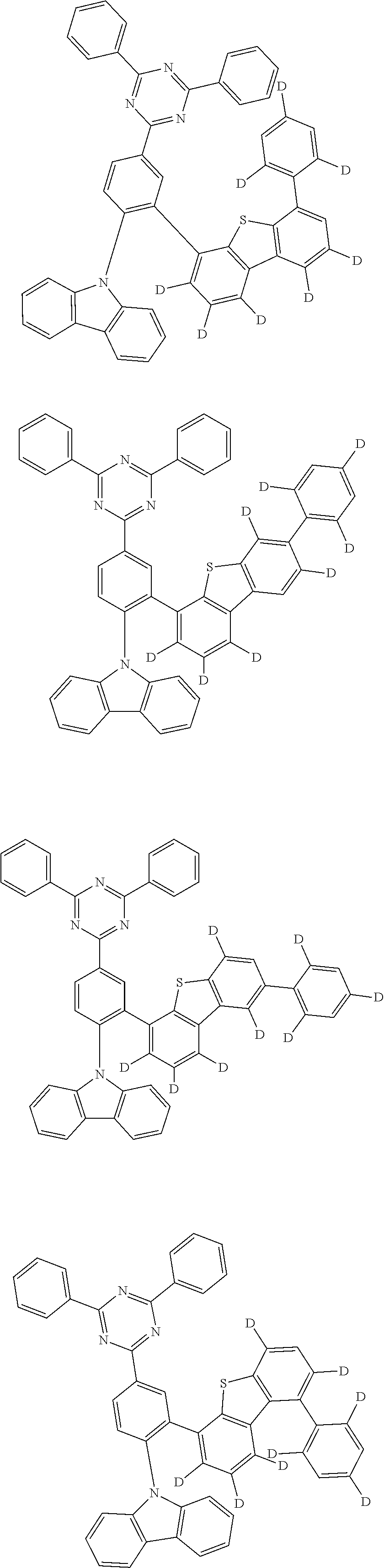

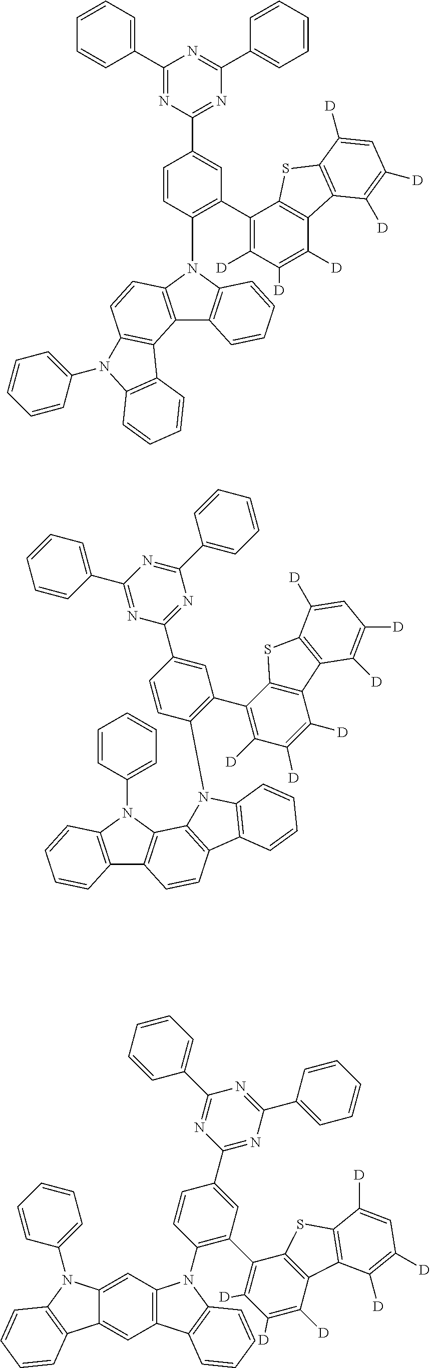 [SMILES: [2H]c1cc([2H])c2sc3c(-c4cc(-c5nc(-c6ccccc6)nc(-c6ccccc6)n5)ccc4-n4c5ccccc5c5c6c7ccccc7n(-c7ccccc7)c6ccc54)c([2H])c([2H])c([2H])c3c2c1[2H] ; [2H]c1cc([2H])c2sc3c(-c4cc(-c5nc(-c6ccccc6)nc(-c6ccccc6)n5)ccc4-n4c5ccccc5c5ccc6c7ccccc7n(-c7ccccc7)c6c54)c([2H])c([2H])c([2H])c3c2c1[2H] ; [2H]c1cc([2H])c2sc3c(-c4cc(-c5nc(-c6ccccc6)nc(-c6ccccc6)n5)ccc4-n4c5ccccc5c5cc6c7ccccc7n(-c7ccccc7)c6cc54)c([2H])c([2H])c([2H])c3c2c1[2H]]

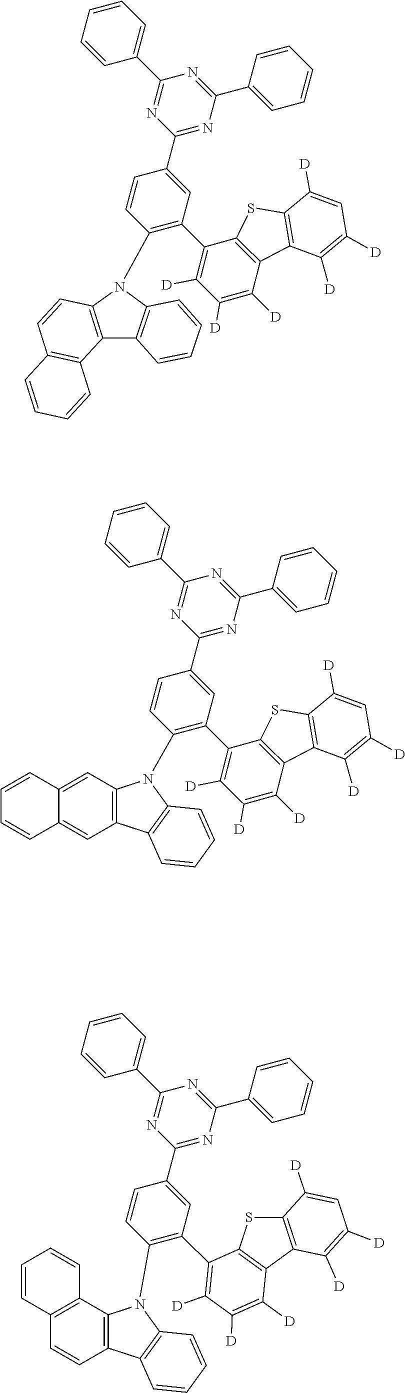 [SMILES: [2H]c1cc([2H])c2sc3c(-c4cc(-c5nc(-c6ccccc6)nc(-c6ccccc6)n5)ccc4-n4c5ccccc5c5c6ccccc6ccc54)c([2H])c([2H])c([2H])c3c2c1[2H] ; [2H]c1cc([2H])c2sc3c(-c4cc(-c5nc(-c6ccccc6)nc(-c6ccccc6)n5)ccc4-n4c5ccccc5c5cc6ccccc6cc54)c([2H])c([2H])c([2H])c3c2c1[2H] ; [2H]c1cc([2H])c2sc3c(-c4cc(-c5nc(-c6ccccc6)nc(-c6ccccc6)n5)ccc4-n4c5ccccc5c5ccc6ccccc6c54)c([2H])c([2H])c([2H])c3c2c1[2H]]

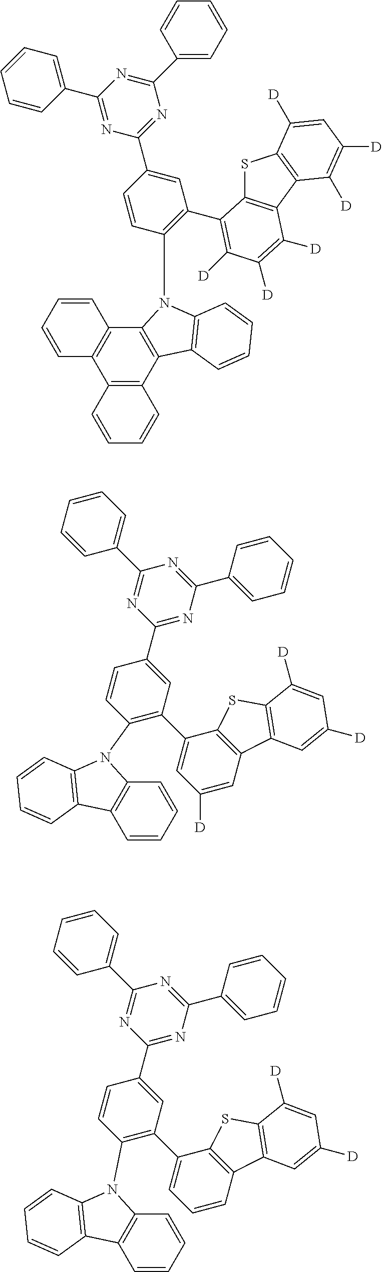 [SMILES: [2H]c1cc([2H])c2sc3c(-c4cc(-c5nc(-c6ccccc6)nc(-c6ccccc6)n5)ccc4-n4c5ccccc5c5c6ccccc6c6ccccc6c54)c([2H])c([2H])c([2H])c3c2c1[2H] ; [2H]c1cc([2H])c2sc3c(-c4cc(-c5nc(-c6ccccc6)nc(-c6ccccc6)n5)ccc4-n4c5ccccc5c5ccccc54)cc([2H])cc3c2c1 ; [2H]c1cc([2H])c2sc3c(-c4cc(-c5nc(-c6ccccc6)nc(-c6ccccc6)n5)ccc4-n4c5ccccc5c5ccccc54)cccc3c2c1]

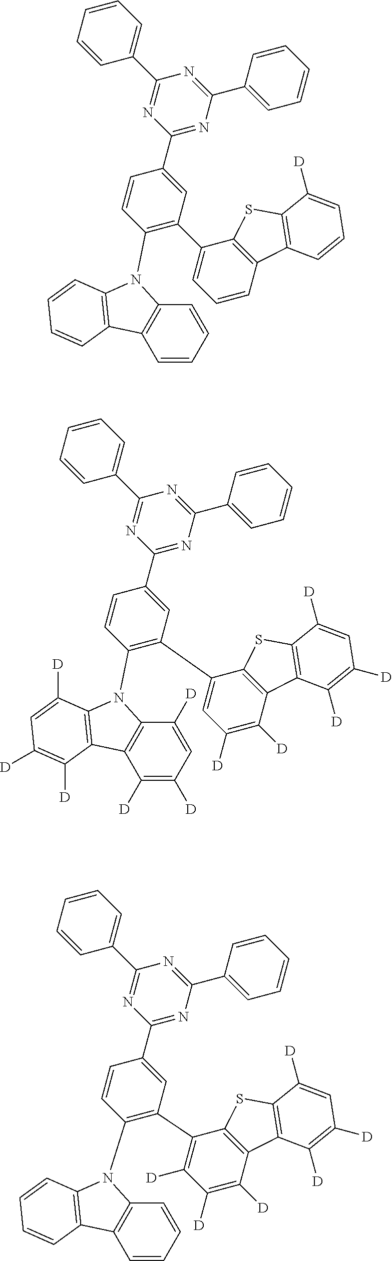 [SMILES: [2H]c1cccc2c1sc1c(-c3cc(-c4nc(-c5ccccc5)nc(-c5ccccc5)n4)ccc3-n3c4ccccc4c4ccccc43)cccc12 ; [2H]c1cc([2H])c2sc3c(-c4cc(-c5nc(-c6ccccc6)nc(-c6ccccc6)n5)ccc4-n4c5c([2H])cc([2H])c([2H])c5c5c([2H])c([2H])cc([2H])c54)cc([2H])c([2H])c3c2c1[2H] ; [2H]c1cc([2H])c2sc3c(-c4cc(-c5nc(-c6ccccc6)nc(-c6ccccc6)n5)ccc4-n4c5ccccc5c5ccccc54)c([2H])c([2H])c([2H])c3c2c1[2H]]

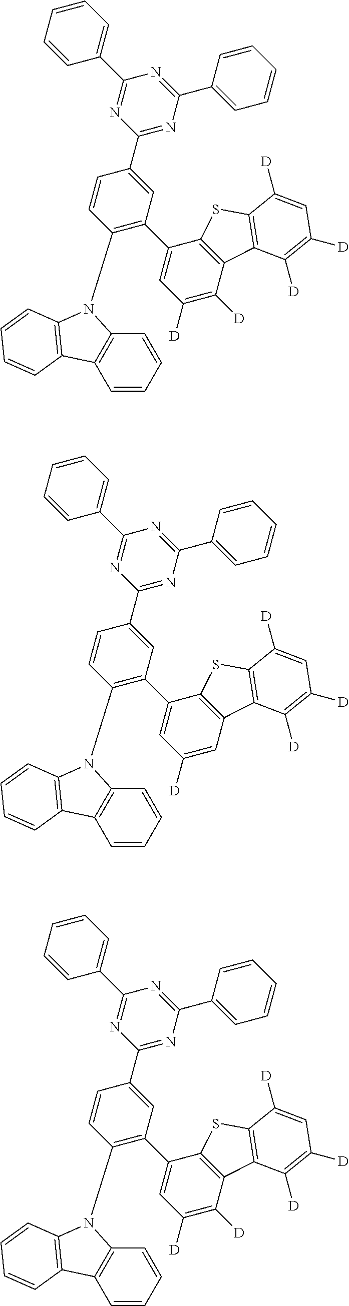 [SMILES: [2H]c1cc([2H])c2sc3c(-c4cc(-c5nc(-c6ccccc6)nc(-c6ccccc6)n5)ccc4-n4c5ccccc5c5ccccc54)cc([2H])c([2H])c3c2c1[2H] ; [2H]c1cc(-c2cc(-c3nc(-c4ccccc4)nc(-c4ccccc4)n3)ccc2-n2c3ccccc3c3ccccc32)c2sc3c([2H])cc([2H])c([2H])c3c2c1 ; [2H]c1cc([2H])c2sc3c(-c4cc(-c5nc(-c6ccccc6)nc(-c6ccccc6)n5)ccc4-n4c5ccccc5c5ccccc54)cc([2H])c([2H])c3c2c1[2H]]

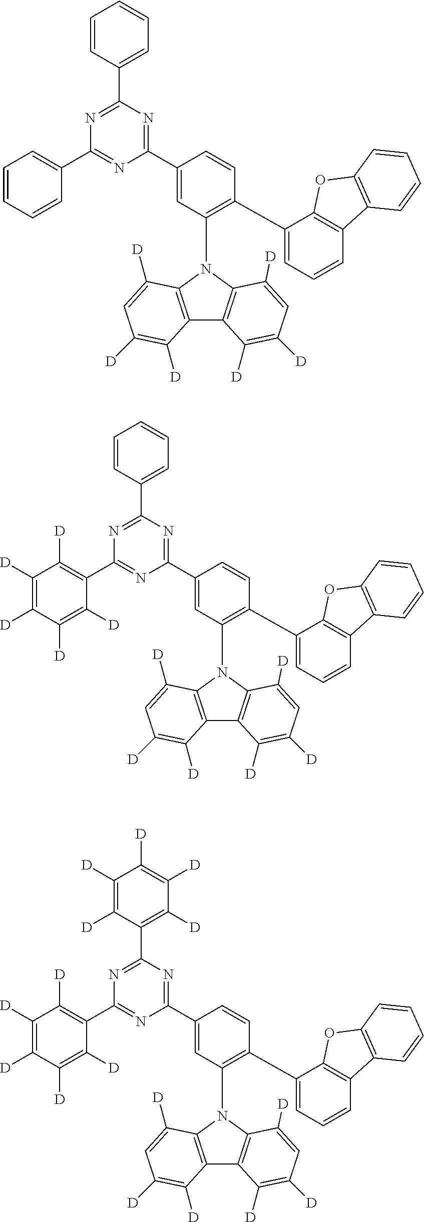 [SMILES: [2H]c1cc([2H])c2c(c1[2H])c1c([2H])c([2H])cc([2H])c1n2-c1cc(-c2nc(-c3ccccc3)nc(-c3ccccc3)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3c([2H])c([2H])c([2H])c([2H])c3[2H])n2)ccc1-c1cccc2c1oc1ccccc12 ; [2H]c1cc([2H])c2c(c1[2H])c1c([2H])c([2H])cc([2H])c1n2-c1cc(-c2nc(-c3c([2H])c([2H])c([2H])c([2H])c3[2H])nc(-c3c([2H])c([2H])c([2H])c([2H])c3[2H])n2)ccc1-c1cccc2c1oc1ccccc12]

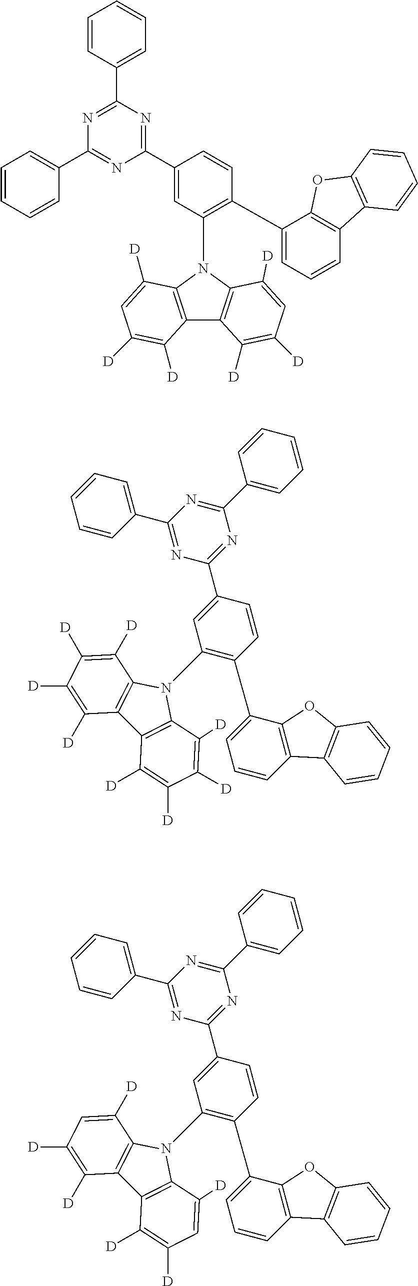 [SMILES: [2H]c1cc([2H])c2c(c1[2H])c1c([2H])c([2H])cc([2H])c1n2-c1cc(-c2nc(-c3ccccc3)nc(-c3ccccc3)n2)ccc1-c1cccc2c1oc1ccccc12 ; [2H]c1c([2H])c([2H])c2c(c1[2H])c1c([2H])c([2H])c([2H])c([2H])c1n2-c1cc(-c2nc(-c3ccccc3)nc(-c3ccccc3)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3ccccc3)n2)ccc1-c1cccc2c1oc1ccccc12]

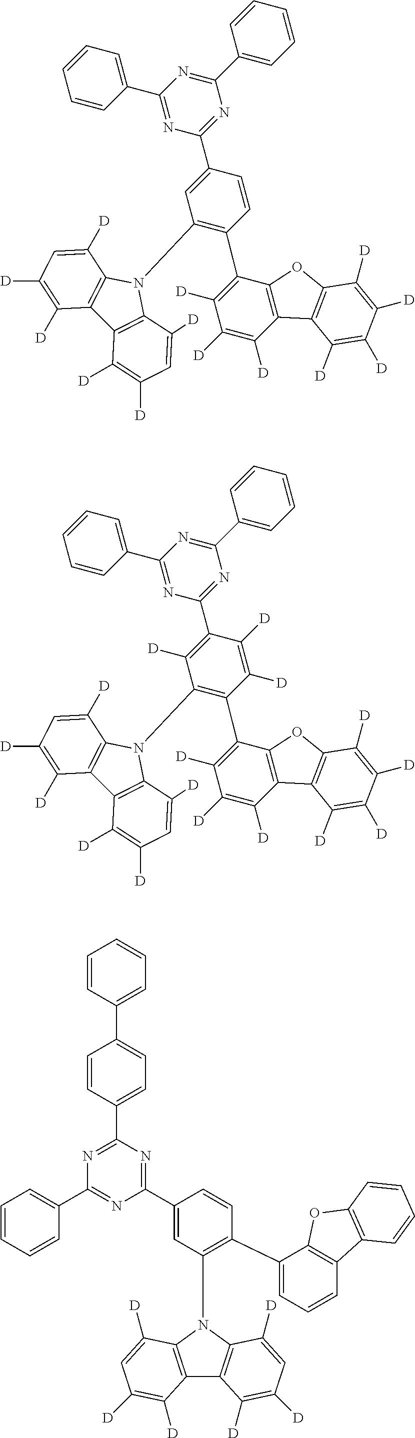 [SMILES: [2H]c1cc([2H])c2c(c1[2H])c1c([2H])c([2H])cc([2H])c1n2-c1cc(-c2nc(-c3ccccc3)nc(-c3ccccc3)n2)ccc1-c1c([2H])c([2H])c([2H])c2c1oc1c([2H])c([2H])c([2H])c([2H])c12 ; [2H]c1cc([2H])c2c(c1[2H])c1c([2H])c([2H])cc([2H])c1n2-c1c([2H])c(-c2nc(-c3ccccc3)nc(-c3ccccc3)n2)c([2H])c([2H])c1-c1c([2H])c([2H])c([2H])c2c1oc1c([2H])c([2H])c([2H])c([2H])c12 ; [2H]c1cc([2H])c2c(c1[2H])c1c([2H])c([2H])cc([2H])c1n2-c1cc(-c2nc(-c3ccccc3)nc(-c3ccc(-c4ccccc4)cc3)n2)ccc1-c1cccc2c1oc1ccccc12]

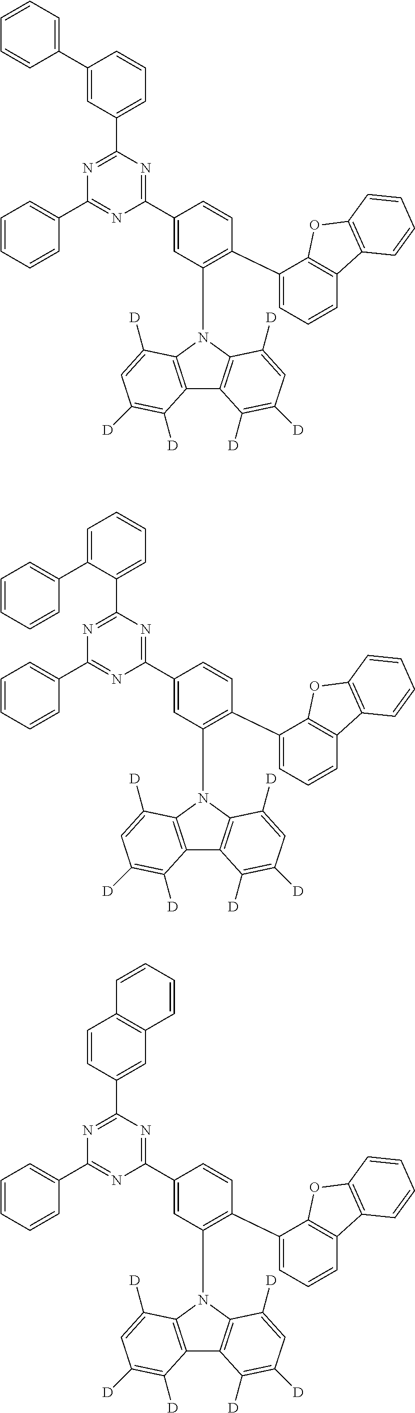 [SMILES: [2H]c1cc([2H])c2c(c1[2H])c1c([2H])c([2H])cc([2H])c1n2-c1cc(-c2nc(-c3ccccc3)nc(-c3cccc(-c4ccccc4)c3)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3ccccc3-c3ccccc3)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3ccc4ccccc4c3)n2)ccc1-c1cccc2c1oc1ccccc12]

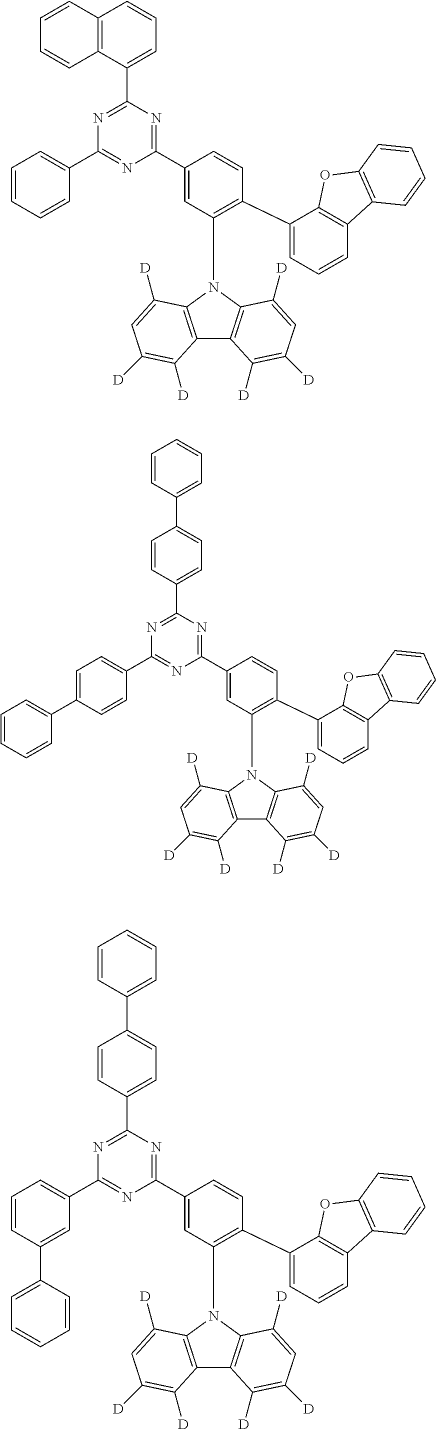

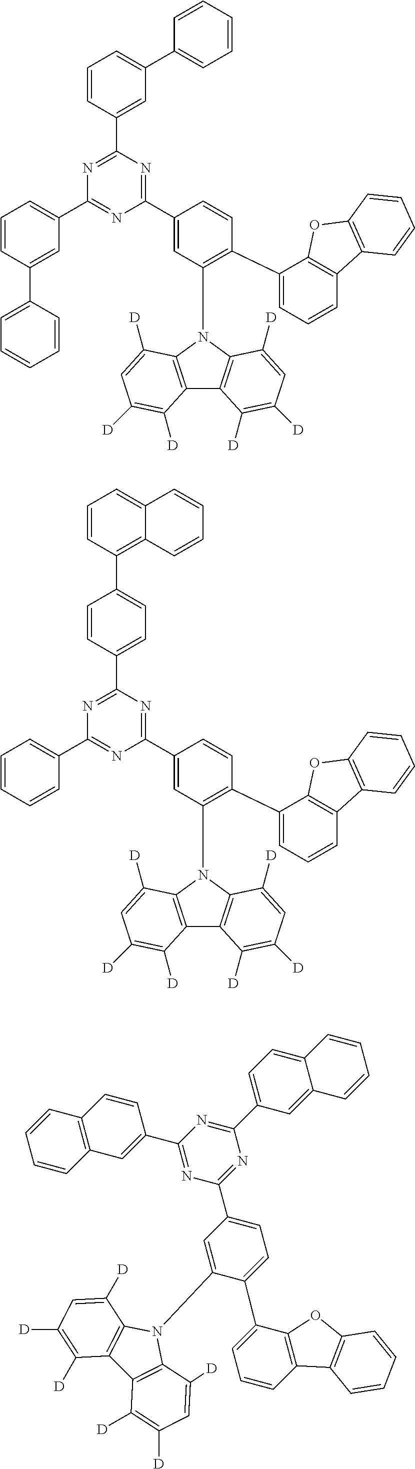

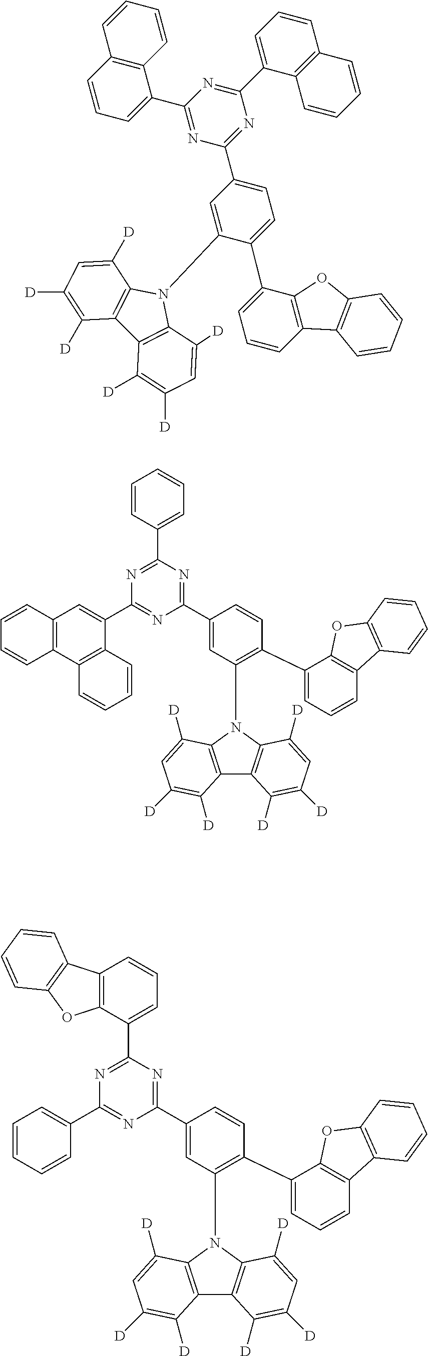

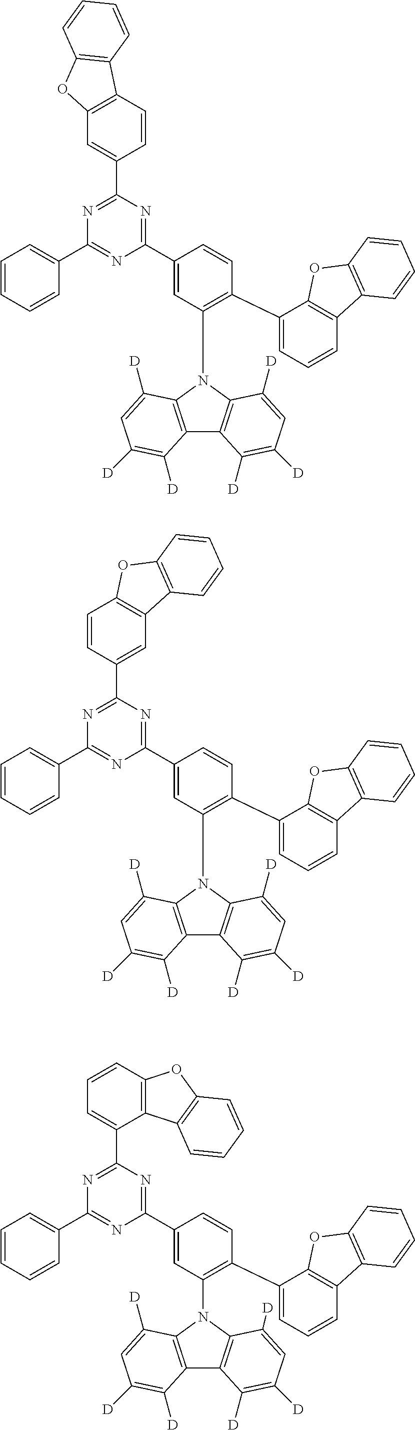

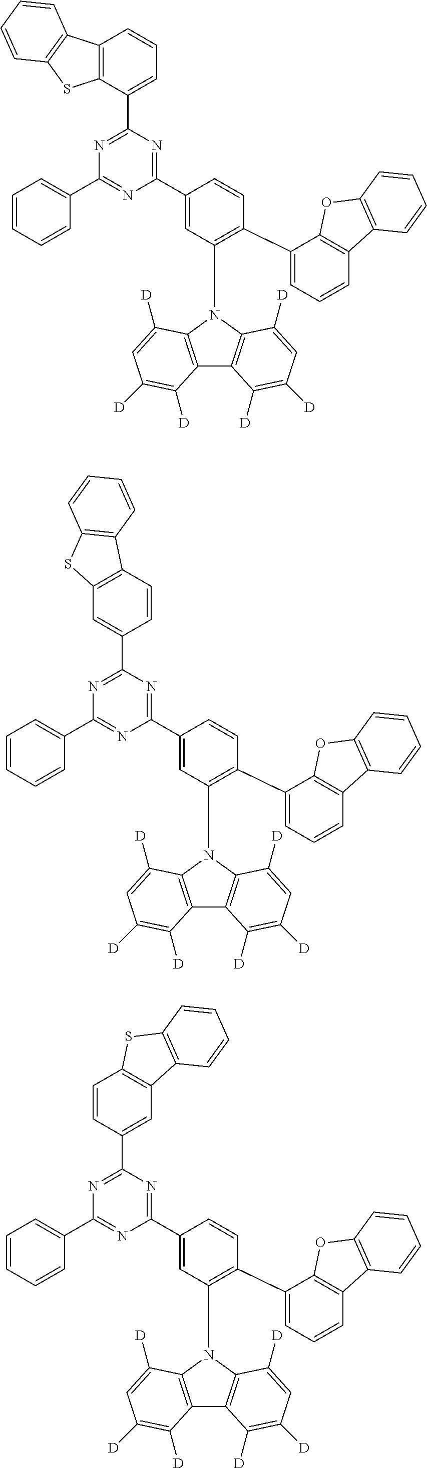

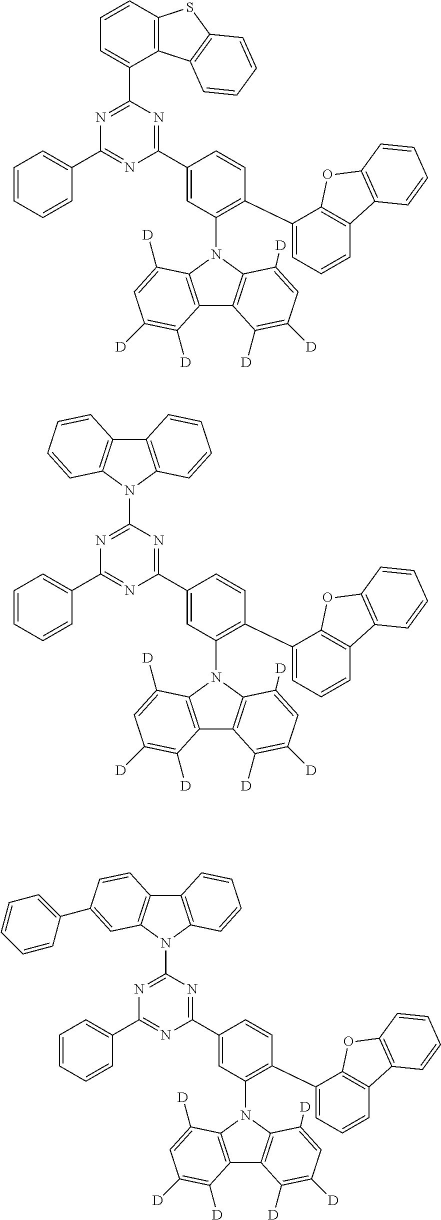 [SMILES: [2H]c1cc([2H])c2c(c1[2H])c1c([2H])c([2H])cc([2H])c1n2-c1cc(-c2nc(-c3ccccc3)nc(-c3cccc4sc5ccccc5c34)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-n3c4ccccc4c4ccccc43)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-n3c4ccccc4c4ccc(-c5ccccc5)cc43)n2)ccc1-c1cccc2c1oc1ccccc12]

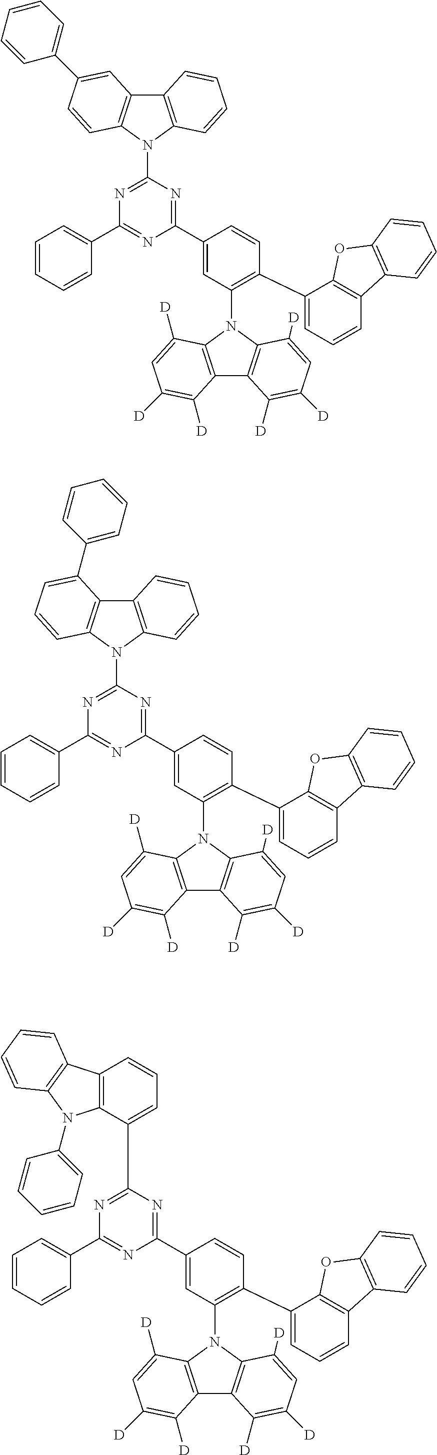 [SMILES: [2H]c1cc([2H])c2c(c1[2H])c1c([2H])c([2H])cc([2H])c1n2-c1cc(-c2nc(-c3ccccc3)nc(-n3c4ccccc4c4cc(-c5ccccc5)ccc43)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-n3c4ccccc4c4c(-c5ccccc5)cccc43)n2)ccc1-c1cccc2c1oc1ccccc12 ; [2H]c1cc([2H])c2c(c1[2H])c1c([2H])c([2H])cc([2H])c1n2-c1cc(-c2nc(-c3ccccc3)nc(-c3cccc4c5ccccc5n(-c5ccccc5)c34)n2)ccc1-c1cccc2c1oc1ccccc12]

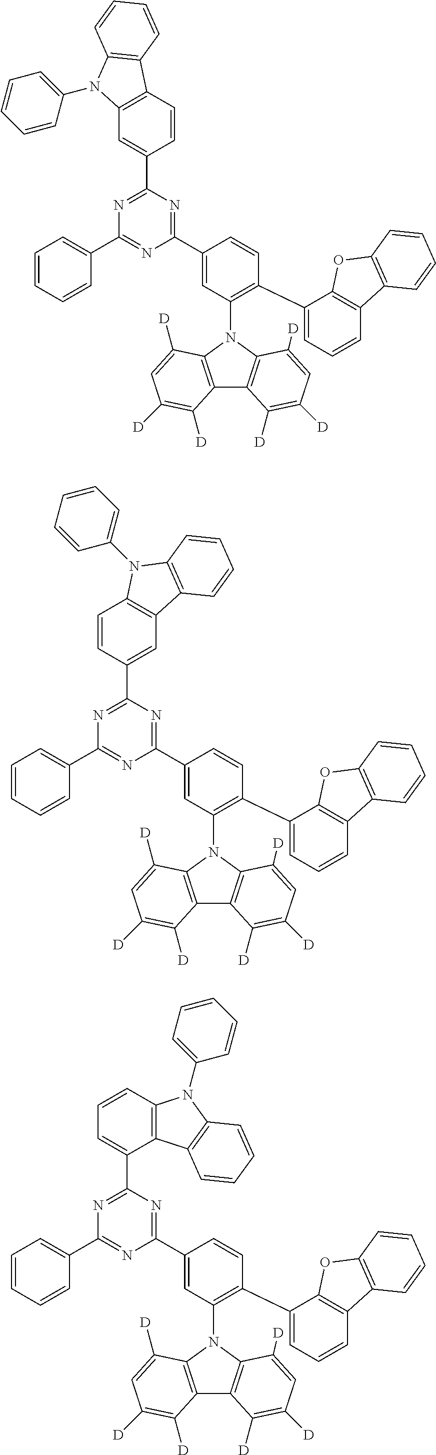

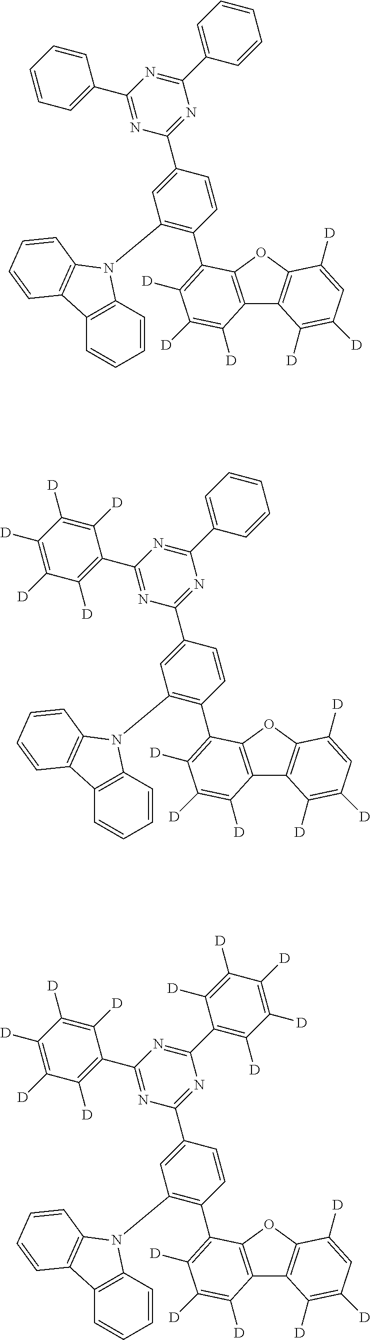 [SMILES: [2H]c1cc([2H])c2oc3c(-c4ccc(-c5nc(-c6ccccc6)nc(-c6ccccc6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6c([2H])c([2H])c([2H])c([2H])c6[2H])n5)cc4-n4c5ccccc5c5ccccc54)c([2H])c([2H])c([2H])c3c2c1[2H] ; [2H]c1cc([2H])c2oc3c(-c4ccc(-c5nc(-c6c([2H])c([2H])c([2H])c([2H])c6[2H])nc(-c6c([2H])c([2H])c([2H])c([2H])c6[2H])n5)cc4-n4c5ccccc5c5ccccc54)c([2H])c([2H])c([2H])c3c2c1[2H]]

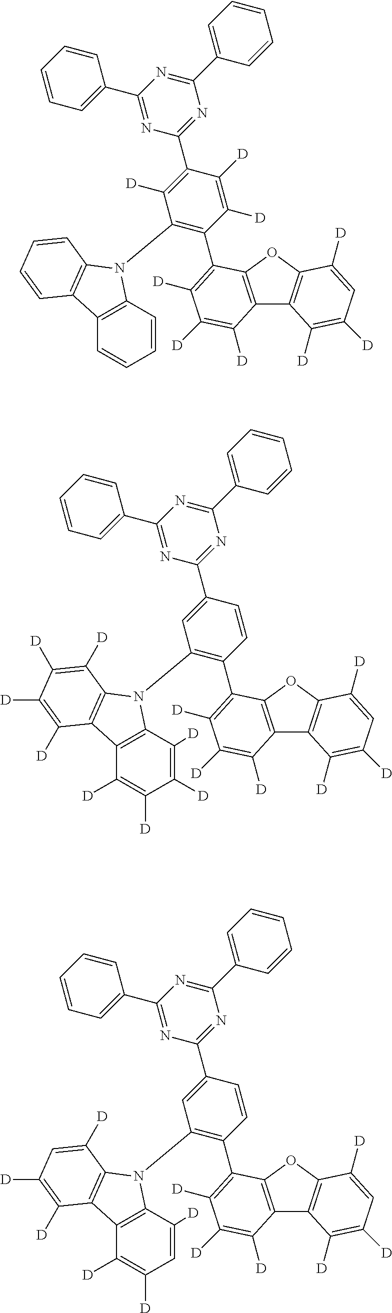 [SMILES: [2H]c1cc([2H])c2oc3c(-c4c([2H])c([2H])c(-c5nc(-c6ccccc6)nc(-c6ccccc6)n5)c([2H])c4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccccc6)n5)cc4-n4c5c([2H])c([2H])c([2H])c([2H])c5c5c([2H])c([2H])c([2H])c([2H])c54)c([2H])c([2H])c([2H])c3c2c1[2H] ; [2H]c1cc([2H])c2oc3c(-c4ccc(-c5nc(-c6ccccc6)nc(-c6ccccc6)n5)cc4-n4c5c([2H])cc([2H])c([2H])c5c5c([2H])c([2H])cc([2H])c54)c([2H])c([2H])c([2H])c3c2c1[2H]]

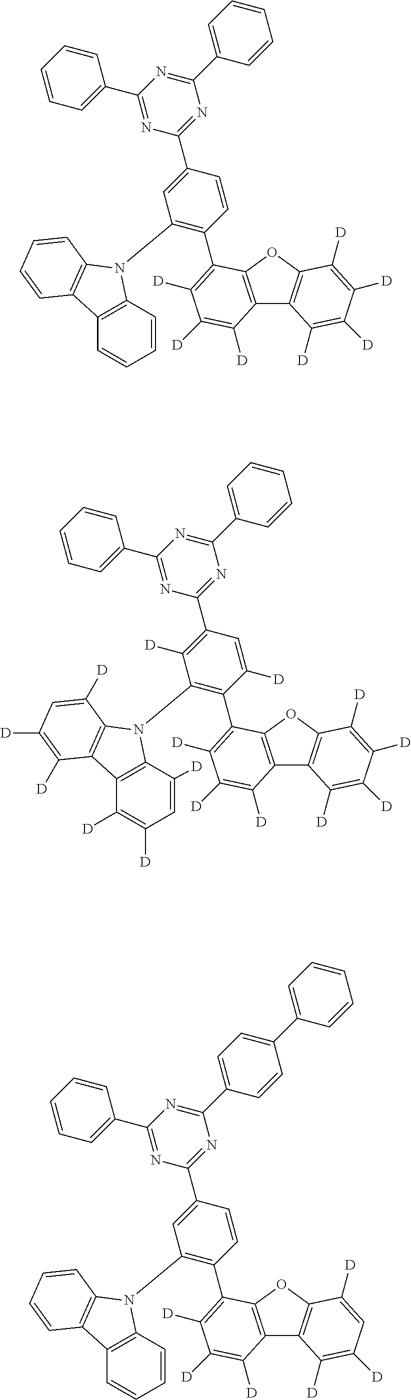 [SMILES: [2H]c1c([2H])c([2H])c2c(oc3c(-c4ccc(-c5nc(-c6ccccc6)nc(-c6ccccc6)n5)cc4-n4c5ccccc5c5ccccc54)c([2H])c([2H])c([2H])c32)c1[2H] ; [2H]c1cc(-c2nc(-c3ccccc3)nc(-c3ccccc3)n2)c([2H])c(-n2c3c([2H])cc([2H])c([2H])c3c3c([2H])c([2H])cc([2H])c32)c1-c1c([2H])c([2H])c([2H])c2c1oc1c([2H])c([2H])c([2H])c([2H])c12 ; [2H]c1cc([2H])c2oc3c(-c4ccc(-c5nc(-c6ccccc6)nc(-c6ccc(-c7ccccc7)cc6)n5)cc4-n4c5ccccc5c5ccccc54)c([2H])c([2H])c([2H])c3c2c1[2H]]

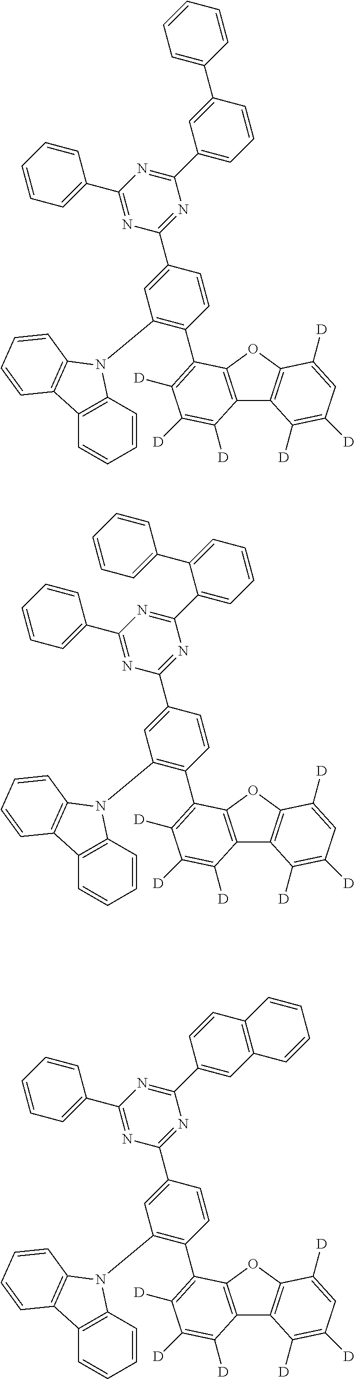 [SMILES: [2H]c1cc([2H])c2oc3c(-c4ccc(-c5nc(-c6ccccc6)nc(-c6cccc(-c7ccccc7)c6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccccc6-c6ccccc6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccc7ccccc7c6)n5)cc4-n4c5ccccc5c5ccccc54)c([2H])c([2H])c([2H])c3c2c1[2H]]

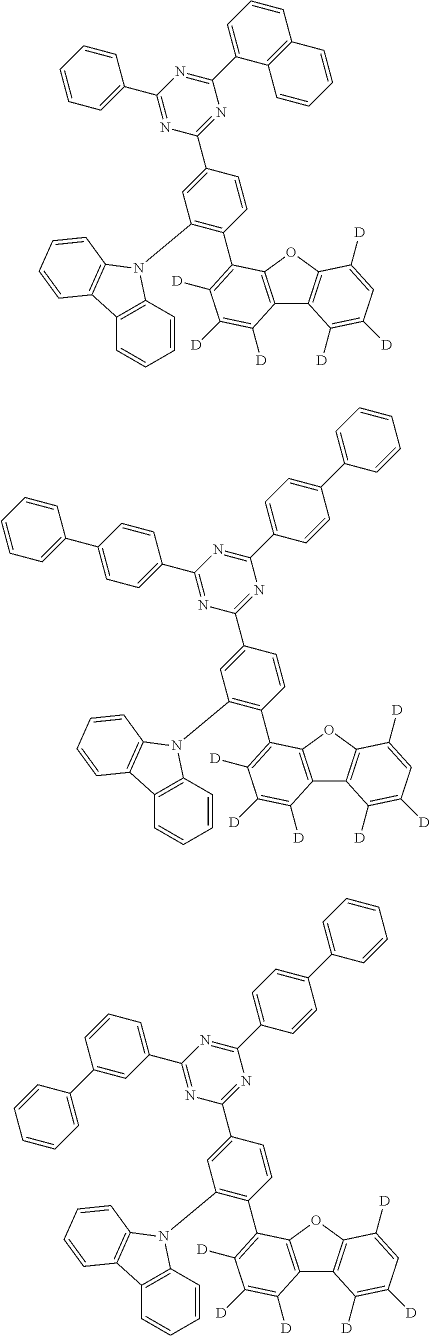 [SMILES: [2H]c1cc([2H])c2oc3c(-c4ccc(-c5nc(-c6ccccc6)nc(-c6cccc7ccccc67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccc(-c7ccccc7)cc6)nc(-c6ccc(-c7ccccc7)cc6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccc(-c7ccccc7)cc6)nc(-c6cccc(-c7ccccc7)c6)n5)cc4-n4c5ccccc5c5ccccc54)c([2H])c([2H])c([2H])c3c2c1[2H]]

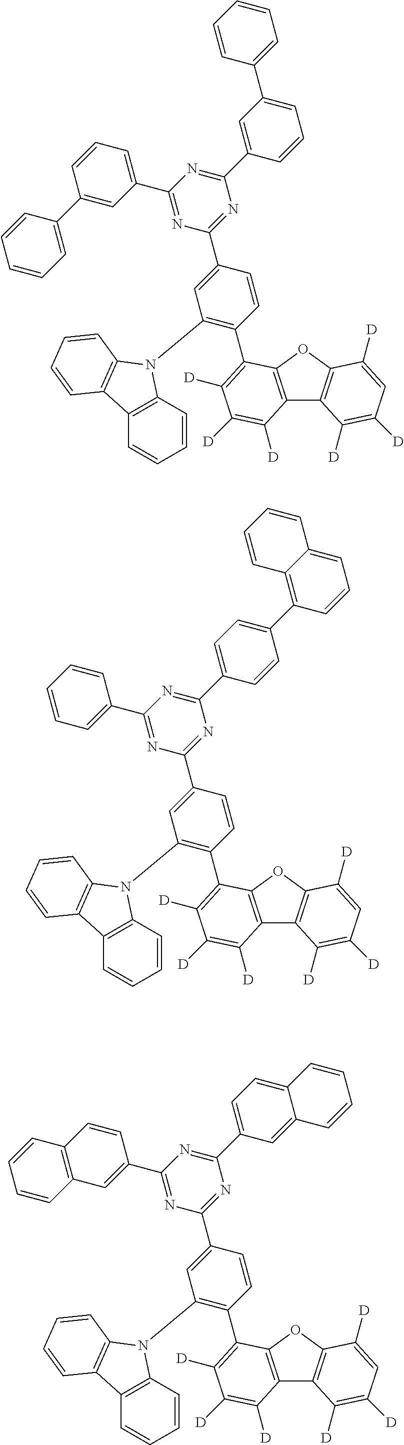 [SMILES: [2H]c1cc([2H])c2oc3c(-c4ccc(-c5nc(-c6cccc(-c7ccccc7)c6)nc(-c6cccc(-c7ccccc7)c6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccc(-c7cccc8ccccc78)cc6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccc7ccccc7c6)nc(-c6ccc7ccccc7c6)n5)cc4-n4c5ccccc5c5ccccc54)c([2H])c([2H])c([2H])c3c2c1[2H]]

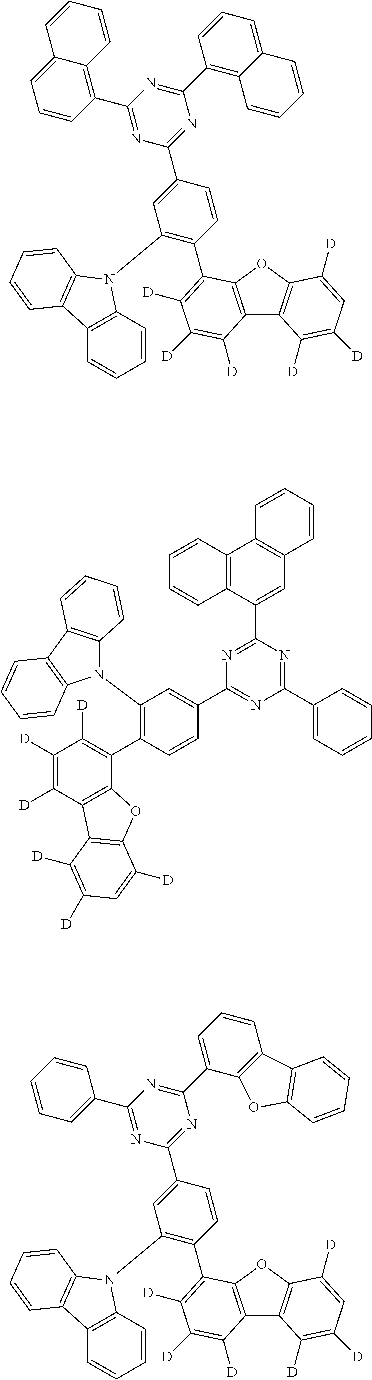 [SMILES: [2H]c1cc([2H])c2oc3c(-c4ccc(-c5nc(-c6cccc7ccccc67)nc(-c6cccc7ccccc67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6cc7ccccc7c7ccccc67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6cccc7c6oc6ccccc67)n5)cc4-n4c5ccccc5c5ccccc54)c([2H])c([2H])c([2H])c3c2c1[2H]]

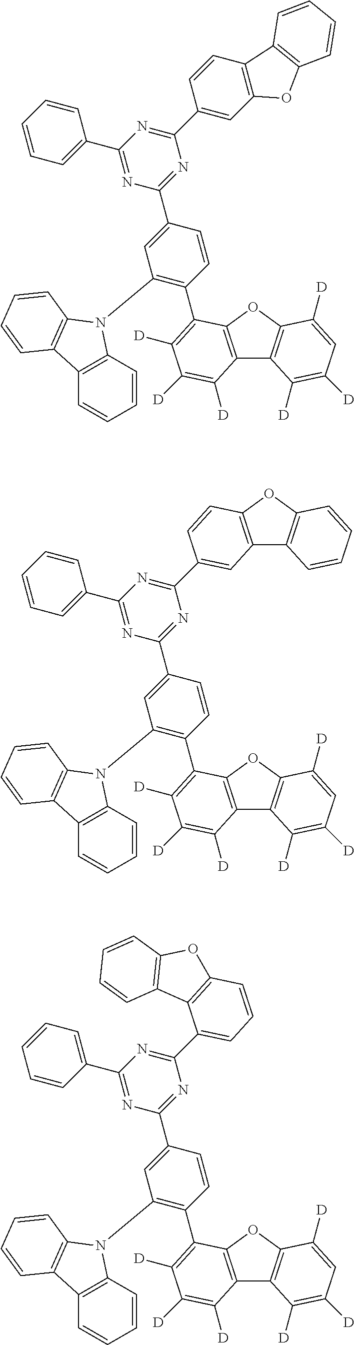 [SMILES: [2H]c1cc([2H])c2oc3c(-c4ccc(-c5nc(-c6ccccc6)nc(-c6ccc7c(c6)oc6ccccc67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccc7oc8ccccc8c7c6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6cccc7oc8ccccc8c67)n5)cc4-n4c5ccccc5c5ccccc54)c([2H])c([2H])c([2H])c3c2c1[2H]]

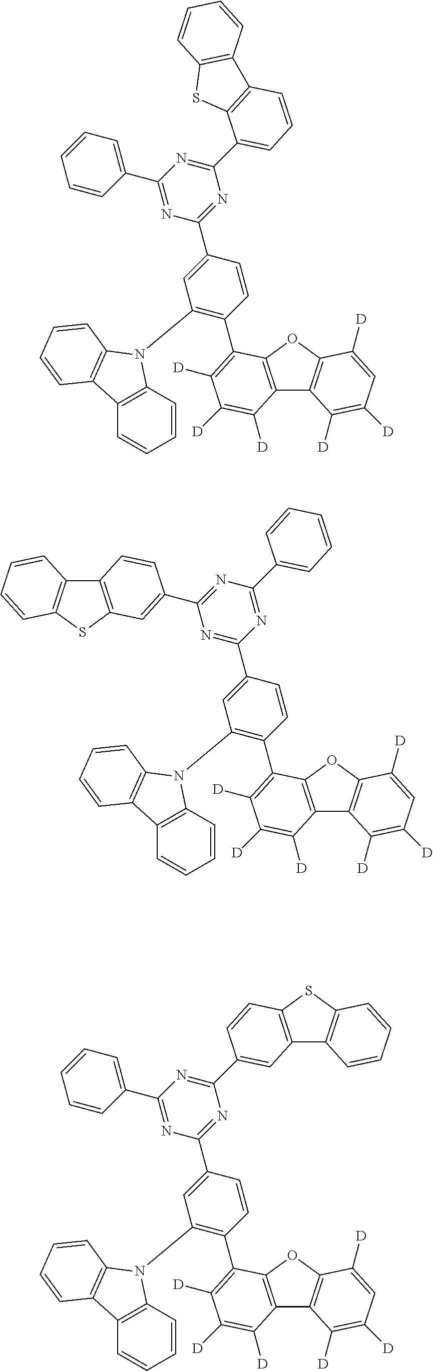 [SMILES: [2H]c1cc([2H])c2oc3c(-c4ccc(-c5nc(-c6ccccc6)nc(-c6cccc7c6sc6ccccc67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccc7c(c6)sc6ccccc67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccc7sc8ccccc8c7c6)n5)cc4-n4c5ccccc5c5ccccc54)c([2H])c([2H])c([2H])c3c2c1[2H]]

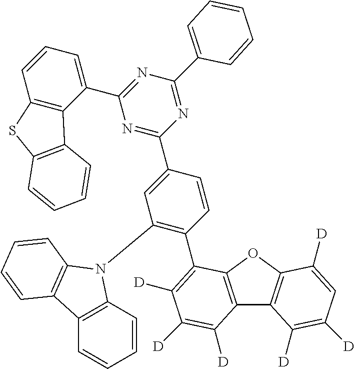 [SMILES: [2H]c1cc([2H])c2oc3c(-c4ccc(-c5nc(-c6ccccc6)nc(-c6cccc7sc8ccccc8c67)n5)cc4-n4c5ccccc5c5ccccc54)c([2H])c([2H])c([2H])c3c2c1[2H]]

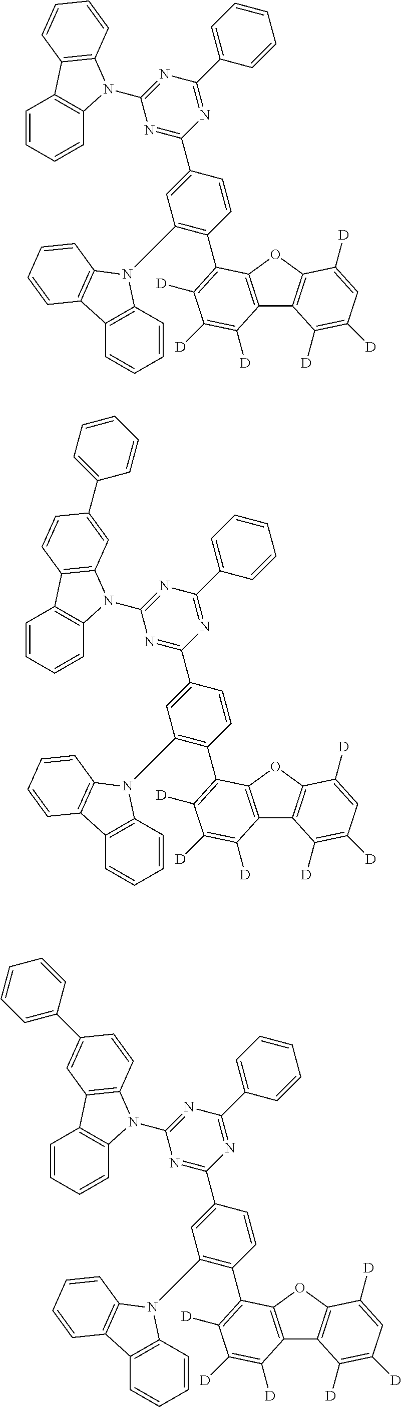 [SMILES: [2H]c1cc([2H])c2oc3c(-c4ccc(-c5nc(-c6ccccc6)nc(-n6c7ccccc7c7ccccc76)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-n6c7ccccc7c7ccc(-c8ccccc8)cc76)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-n6c7ccccc7c7cc(-c8ccccc8)ccc76)n5)cc4-n4c5ccccc5c5ccccc54)c([2H])c([2H])c([2H])c3c2c1[2H]]

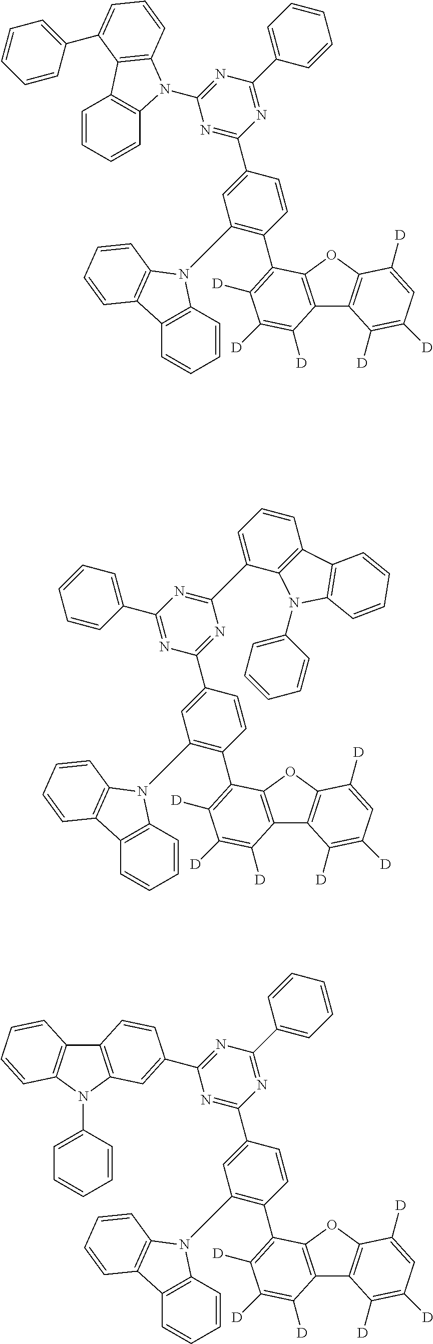 [SMILES: [2H]c1cc([2H])c2oc3c(-c4ccc(-c5nc(-c6ccccc6)nc(-n6c7ccccc7c7c(-c8ccccc8)cccc76)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6cccc7c8ccccc8n(-c8ccccc8)c67)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6ccc7c8ccccc8n(-c8ccccc8)c7c6)n5)cc4-n4c5ccccc5c5ccccc54)c([2H])c([2H])c([2H])c3c2c1[2H]]

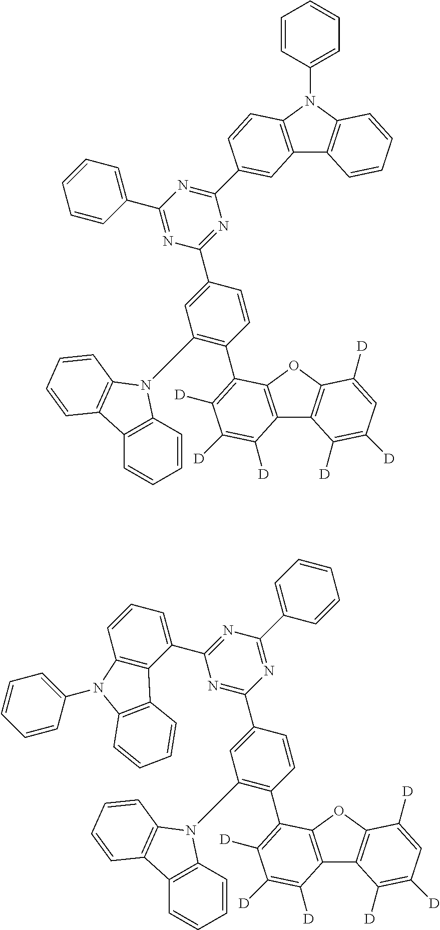 [SMILES: [2H]c1cc([2H])c2oc3c(-c4ccc(-c5nc(-c6ccccc6)nc(-c6ccc7c(c6)c6ccccc6n7-c6ccccc6)n5)cc4-n4c5ccccc5c5ccccc54)c([2H])c([2H])c([2H])c3c2c1[2H] ; [2H]c1cc([2H])c2oc3c(-c4ccc(-c5nc(-c6ccccc6)nc(-c6cccc7c6c6ccccc6n7-c6ccccc6)n5)cc4-n4c5ccccc5c5ccccc54)c([2H])c([2H])c([2H])c3c2c1[2H]]

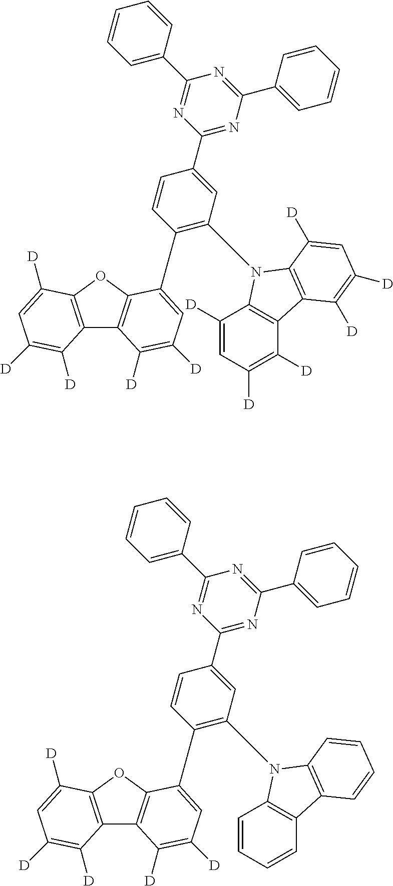 [SMILES: [2H]c1cc([2H])c2oc3c(-c4ccc(-c5nc(-c6ccccc6)nc(-c6ccccc6)n5)cc4-n4c5c([2H])cc([2H])c([2H])c5c5c([2H])c([2H])cc([2H])c54)cc([2H])c([2H])c3c2c1[2H] ; [2H]c1cc([2H])c2oc3c(-c4ccc(-c5nc(-c6ccccc6)nc(-c6ccccc6)n5)cc4-n4c5ccccc5c5ccccc54)cc([2H])c([2H])c3c2c1[2H]]

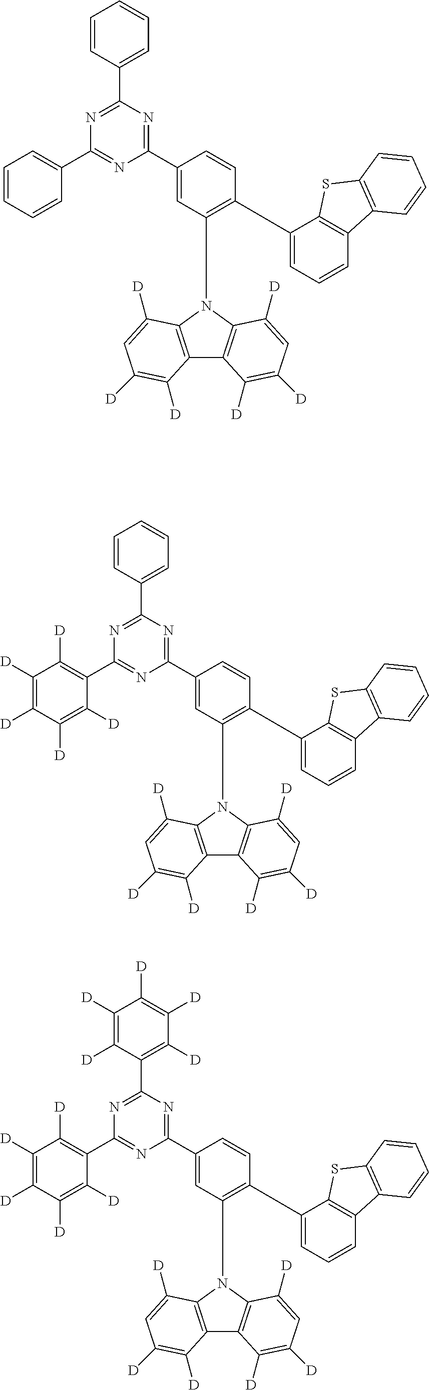 [SMILES: [2H]c1cc([2H])c2c(c1[2H])c1c([2H])c([2H])cc([2H])c1n2-c1cc(-c2nc(-c3ccccc3)nc(-c3ccccc3)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3c([2H])c([2H])c([2H])c([2H])c3[2H])n2)ccc1-c1cccc2c1sc1ccccc12 ; [2H]c1cc([2H])c2c(c1[2H])c1c([2H])c([2H])cc([2H])c1n2-c1cc(-c2nc(-c3c([2H])c([2H])c([2H])c([2H])c3[2H])nc(-c3c([2H])c([2H])c([2H])c([2H])c3[2H])n2)ccc1-c1cccc2c1sc1ccccc12]

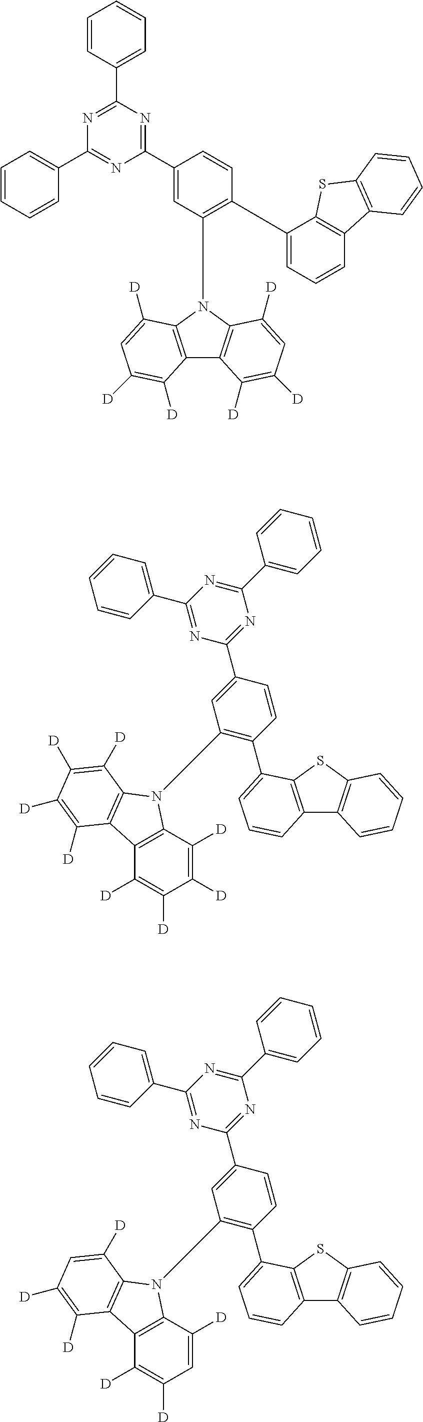 [SMILES: [2H]c1cc([2H])c2c(c1[2H])c1c([2H])c([2H])cc([2H])c1n2-c1cc(-c2nc(-c3ccccc3)nc(-c3ccccc3)n2)ccc1-c1cccc2c1sc1ccccc12 ; [2H]c1c([2H])c([2H])c2c(c1[2H])c1c([2H])c([2H])c([2H])c([2H])c1n2-c1cc(-c2nc(-c3ccccc3)nc(-c3ccccc3)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3ccccc3)n2)ccc1-c1cccc2c1sc1ccccc12]

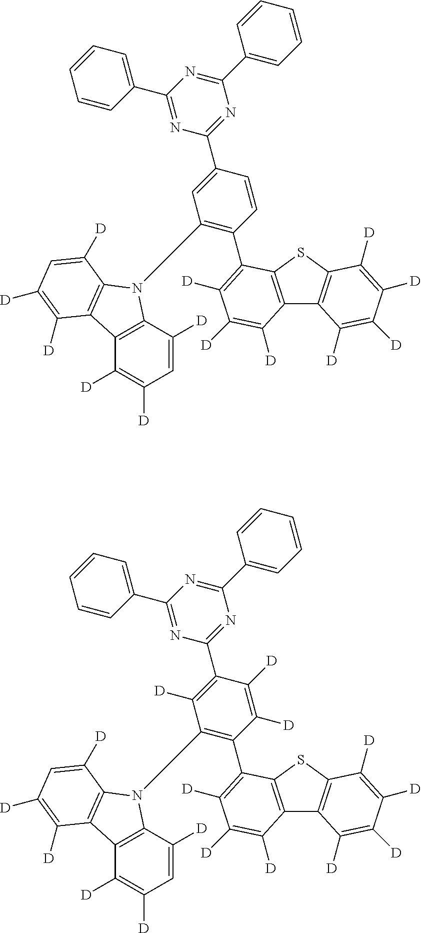 [SMILES: [2H]c1cc([2H])c2c(c1[2H])c1c([2H])c([2H])cc([2H])c1n2-c1cc(-c2nc(-c3ccccc3)nc(-c3ccccc3)n2)ccc1-c1c([2H])c([2H])c([2H])c2c1sc1c([2H])c([2H])c([2H])c([2H])c12 ; [2H]c1cc([2H])c2c(c1[2H])c1c([2H])c([2H])cc([2H])c1n2-c1c([2H])c(-c2nc(-c3ccccc3)nc(-c3ccccc3)n2)c([2H])c([2H])c1-c1c([2H])c([2H])c([2H])c2c1sc1c([2H])c([2H])c([2H])c([2H])c12]

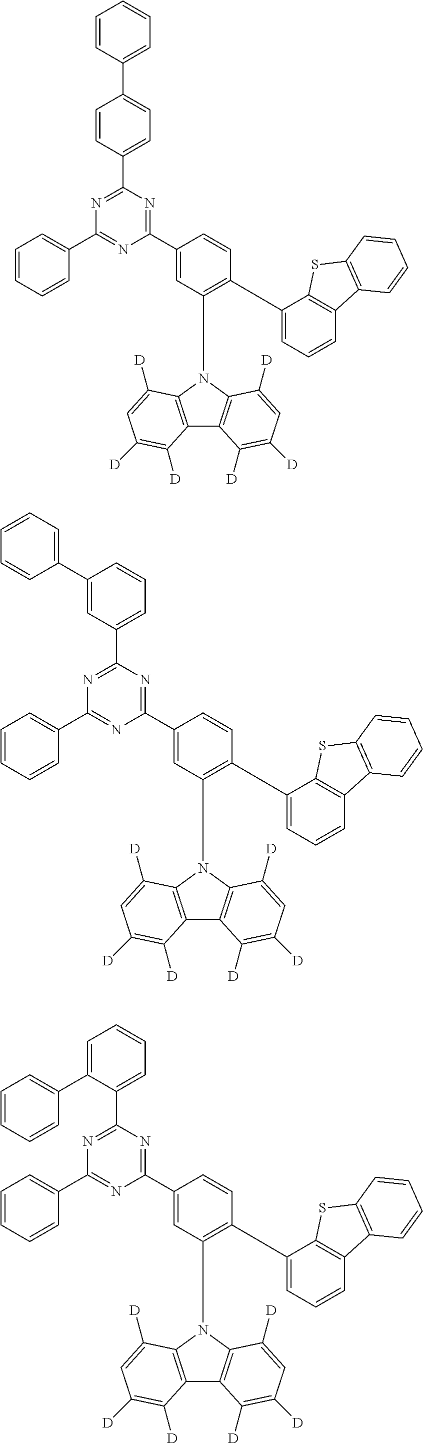 [SMILES: [2H]c1cc([2H])c2c(c1[2H])c1c([2H])c([2H])cc([2H])c1n2-c1cc(-c2nc(-c3ccccc3)nc(-c3ccc(-c4ccccc4)cc3)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3cccc(-c4ccccc4)c3)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3ccccc3-c3ccccc3)n2)ccc1-c1cccc2c1sc1ccccc12]

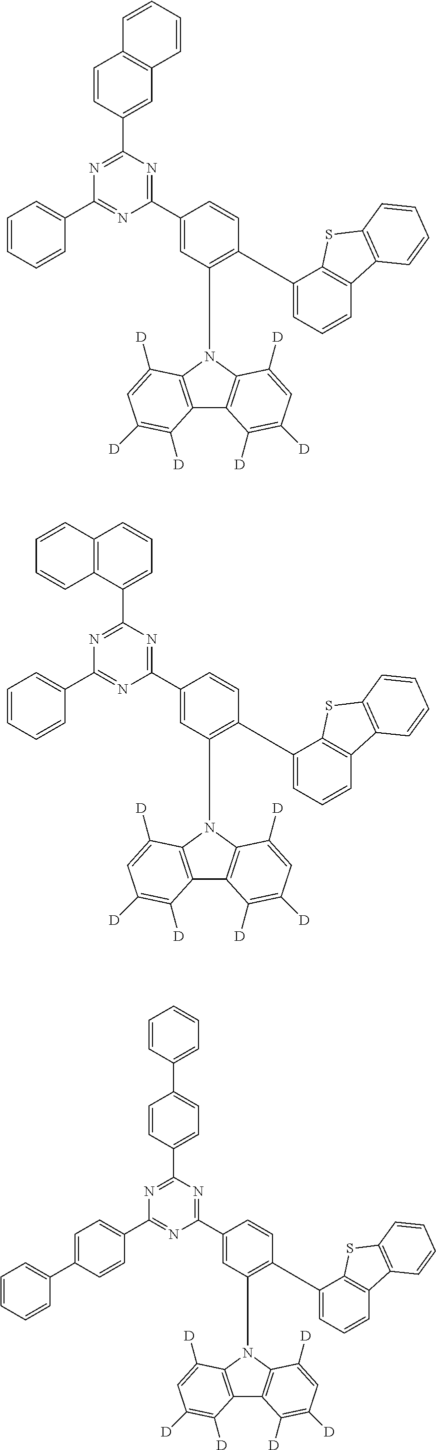 [SMILES: [2H]c1cc([2H])c2c(c1[2H])c1c([2H])c([2H])cc([2H])c1n2-c1cc(-c2nc(-c3ccccc3)nc(-c3ccc4ccccc4c3)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3cccc4ccccc34)n2)ccc1-c1cccc2c1sc1ccccc12 ; [2H]c1cc([2H])c2c(c1[2H])c1c([2H])c([2H])cc([2H])c1n2-c1cc(-c2nc(-c3ccc(-c4ccccc4)cc3)nc(-c3ccc(-c4ccccc4)cc3)n2)ccc1-c1cccc2c1sc1ccccc12]

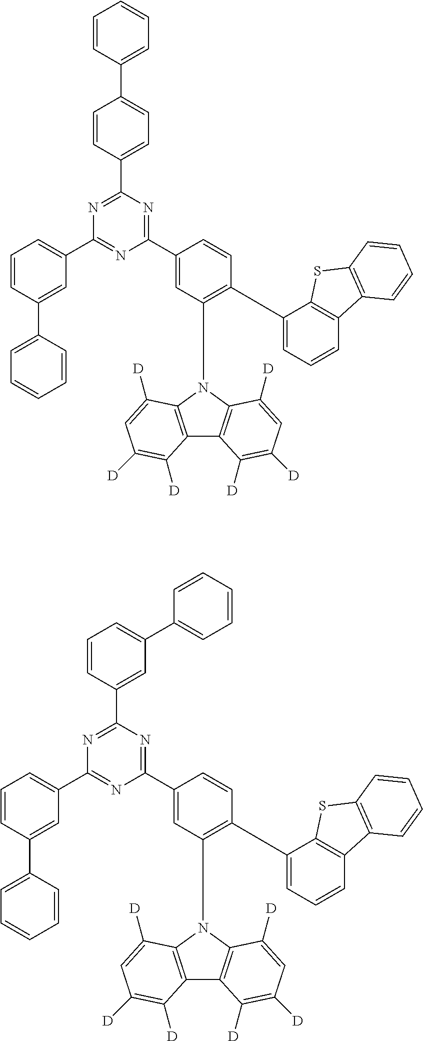 [SMILES: [2H]c1cc([2H])c2c(c1[2H])c1c([2H])c([2H])cc([2H])c1n2-c1cc(-c2nc(-c3ccc(-c4ccccc4)cc3)nc(-c3cccc(-c4ccccc4)c3)n2)ccc1-c1cccc2c1sc1ccccc12 ; [2H]c1cc([2H])c2c(c1[2H])c1c([2H])c([2H])cc([2H])c1n2-c1cc(-c2nc(-c3cccc(-c4ccccc4)c3)nc(-c3cccc(-c4ccccc4)c3)n2)ccc1-c1cccc2c1sc1ccccc12]

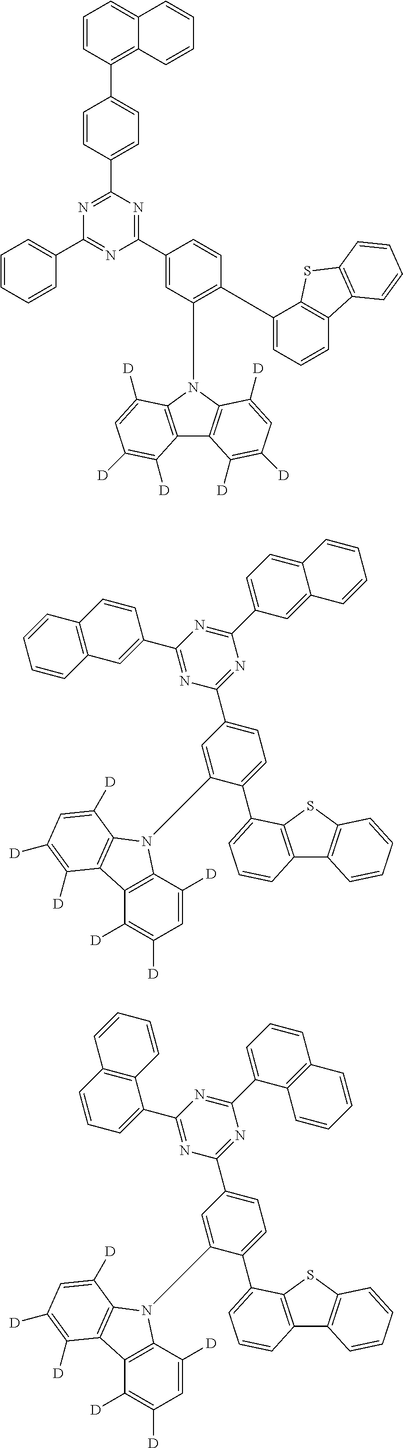 [SMILES: [2H]c1cc([2H])c2c(c1[2H])c1c([2H])c([2H])cc([2H])c1n2-c1cc(-c2nc(-c3ccccc3)nc(-c3ccc(-c4cccc5ccccc45)cc3)n2)ccc1-c1cccc2c1sc1ccccc12 ; [2H]c1cc([2H])c2c(c1[2H])c1c([2H])c([2H])cc([2H])c1n2-c1cc(-c2nc(-c3ccc4ccccc4c3)nc(-c3ccc4ccccc4c3)n2)ccc1-c1cccc2c1sc1ccccc12 ; [2H]c1cc([2H])c2c(c1[2H])c1c([2H])c([2H])cc([2H])c1n2-c1cc(-c2nc(-c3cccc4ccccc34)nc(-c3cccc4ccccc34)n2)ccc1-c1cccc2c1sc1ccccc12]

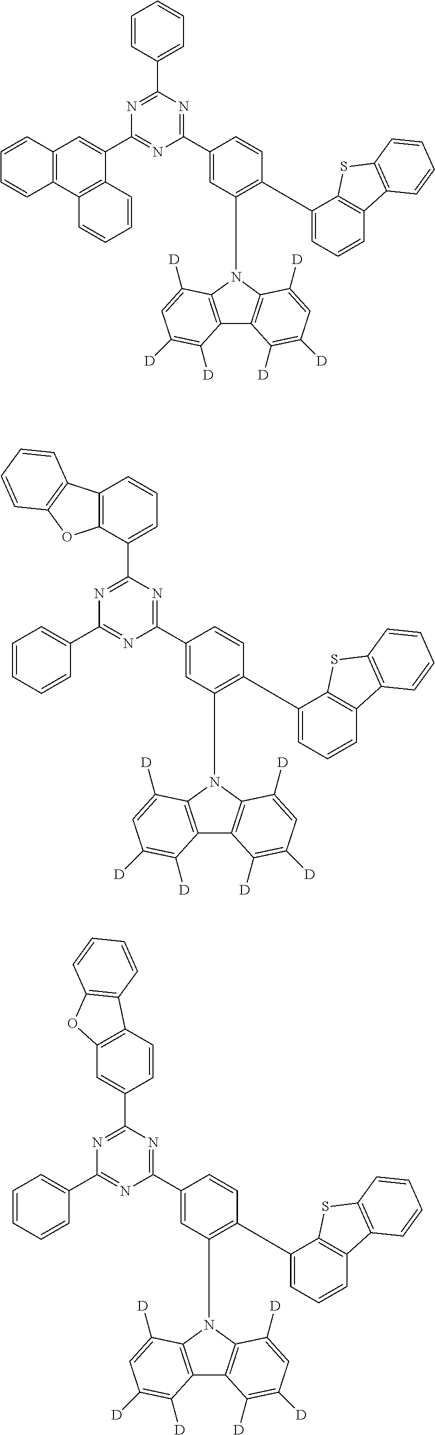 [SMILES: [2H]c1cc([2H])c2c(c1[2H])c1c([2H])c([2H])cc([2H])c1n2-c1cc(-c2nc(-c3ccccc3)nc(-c3cc4ccccc4c4ccccc34)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3cccc4c3oc3ccccc34)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3ccc4c(c3)oc3ccccc34)n2)ccc1-c1cccc2c1sc1ccccc12]

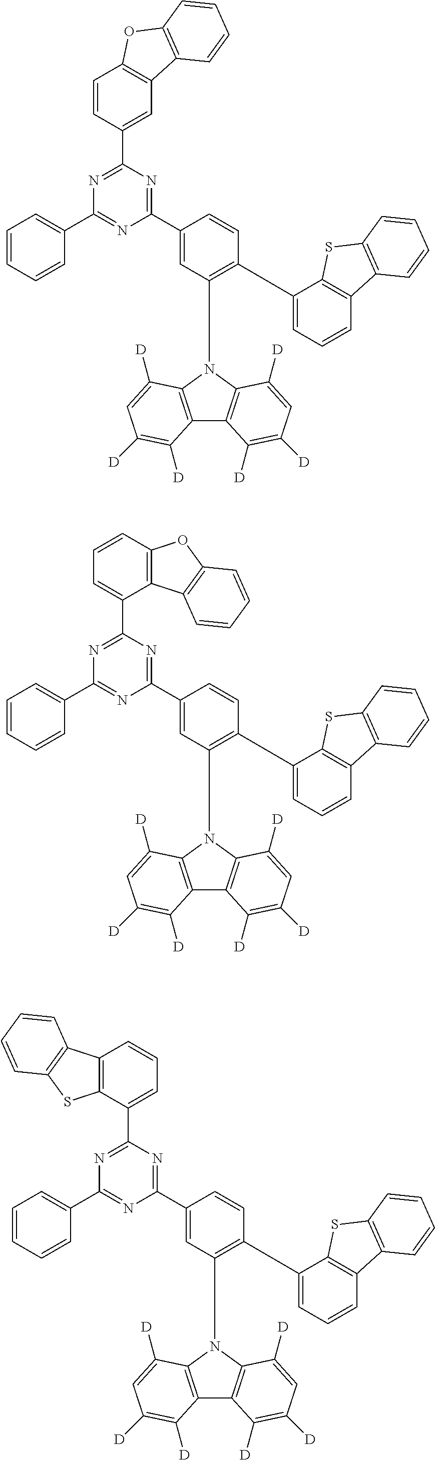 [SMILES: [2H]c1cc([2H])c2c(c1[2H])c1c([2H])c([2H])cc([2H])c1n2-c1cc(-c2nc(-c3ccccc3)nc(-c3ccc4oc5ccccc5c4c3)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3cccc4oc5ccccc5c34)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3cccc4c3sc3ccccc34)n2)ccc1-c1cccc2c1sc1ccccc12]

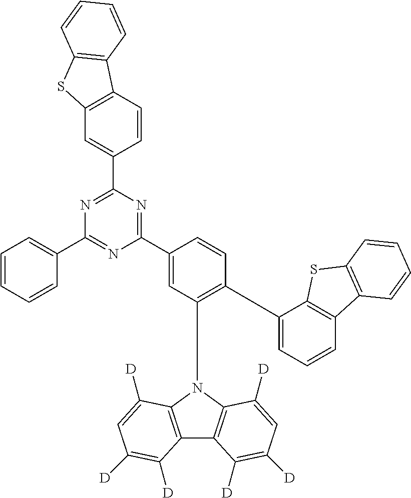 [SMILES: [2H]c1cc([2H])c2c(c1[2H])c1c([2H])c([2H])cc([2H])c1n2-c1cc(-c2nc(-c3ccccc3)nc(-c3ccc4c(c3)sc3ccccc34)n2)ccc1-c1cccc2c1sc1ccccc12]

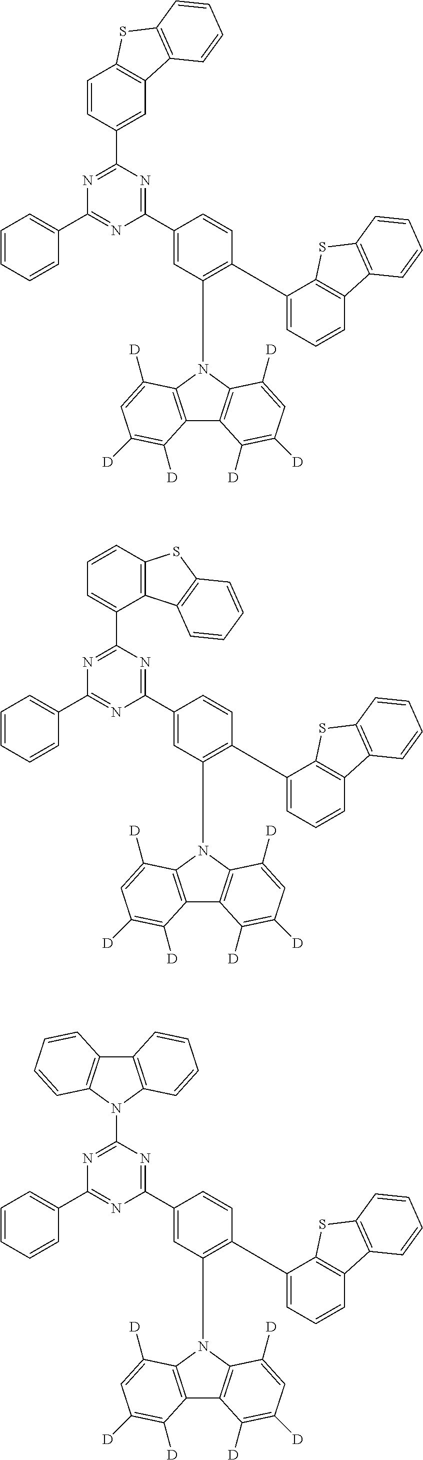 [SMILES: [2H]c1cc([2H])c2c(c1[2H])c1c([2H])c([2H])cc([2H])c1n2-c1cc(-c2nc(-c3ccccc3)nc(-c3ccc4sc5ccccc5c4c3)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3cccc4sc5ccccc5c34)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-n3c4ccccc4c4ccccc43)n2)ccc1-c1cccc2c1sc1ccccc12]

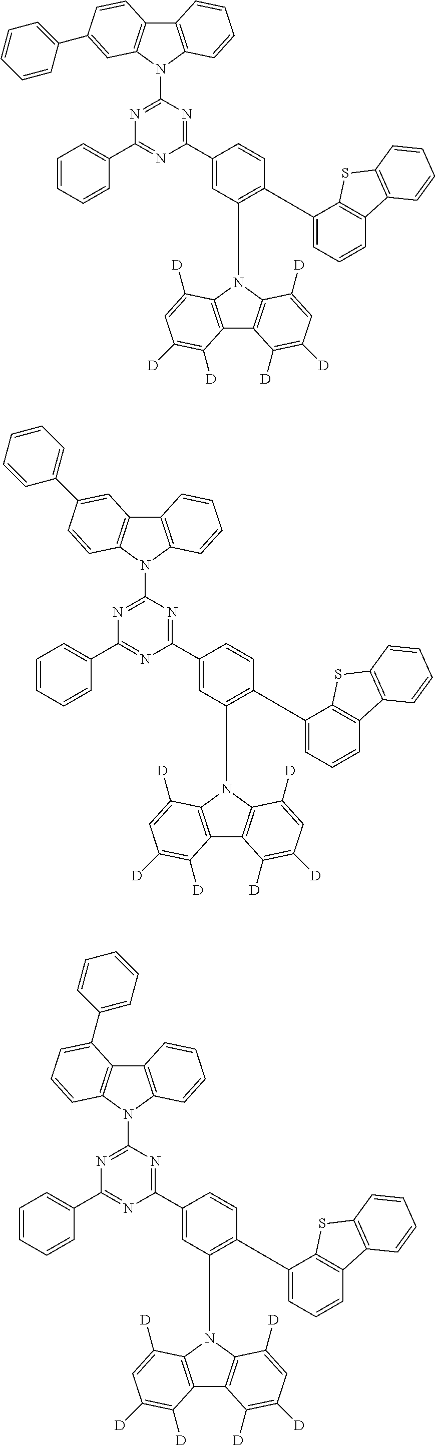 [SMILES: [2H]c1cc([2H])c2c(c1[2H])c1c([2H])c([2H])cc([2H])c1n2-c1cc(-c2nc(-c3ccccc3)nc(-n3c4ccccc4c4ccc(-c5ccccc5)cc43)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-n3c4ccccc4c4cc(-c5ccccc5)ccc43)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-n3c4ccccc4c4c(-c5ccccc5)cccc43)n2)ccc1-c1cccc2c1sc1ccccc12]

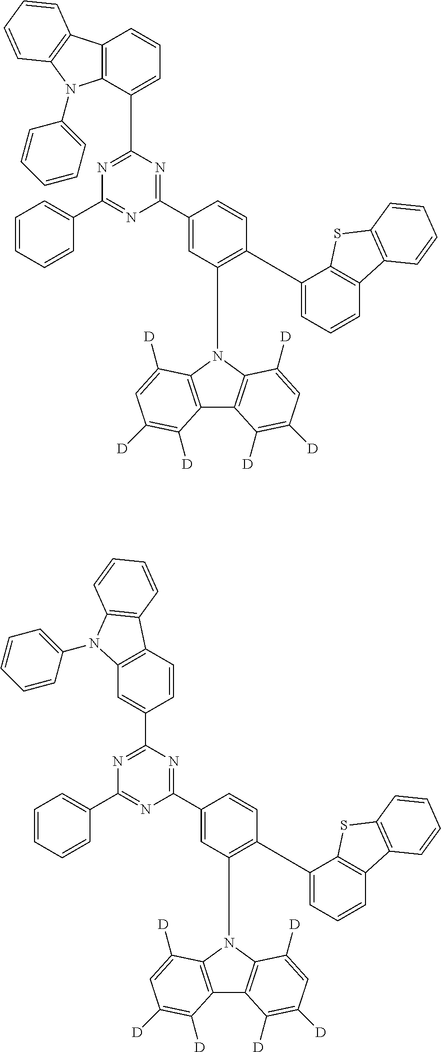 [SMILES: [2H]c1cc([2H])c2c(c1[2H])c1c([2H])c([2H])cc([2H])c1n2-c1cc(-c2nc(-c3ccccc3)nc(-c3cccc4c5ccccc5n(-c5ccccc5)c34)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3ccc4c5ccccc5n(-c5ccccc5)c4c3)n2)ccc1-c1cccc2c1sc1ccccc12]

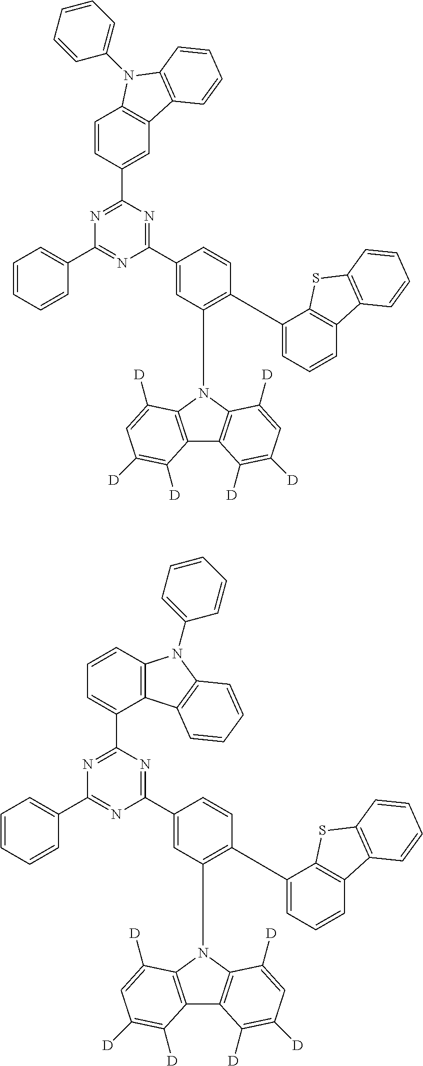 [SMILES: [2H]c1cc([2H])c2c(c1[2H])c1c([2H])c([2H])cc([2H])c1n2-c1cc(-c2nc(-c3ccccc3)nc(-c3ccc4c(c3)c3ccccc3n4-c3ccccc3)n2)ccc1-c1cccc2c1sc1ccccc12 ; [2H]c1cc([2H])c2c(c1[2H])c1c([2H])c([2H])cc([2H])c1n2-c1cc(-c2nc(-c3ccccc3)nc(-c3cccc4c3c3ccccc3n4-c3ccccc3)n2)ccc1-c1cccc2c1sc1ccccc12]

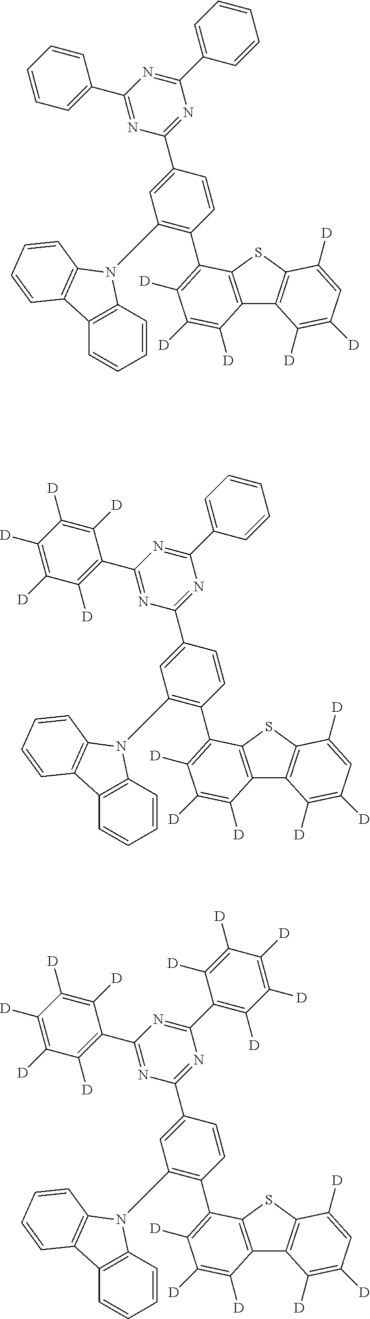 [SMILES: [2H]c1cc([2H])c2sc3c(-c4ccc(-c5nc(-c6ccccc6)nc(-c6ccccc6)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6c([2H])c([2H])c([2H])c([2H])c6[2H])n5)cc4-n4c5ccccc5c5ccccc54)c([2H])c([2H])c([2H])c3c2c1[2H] ; [2H]c1cc([2H])c2sc3c(-c4ccc(-c5nc(-c6c([2H])c([2H])c([2H])c([2H])c6[2H])nc(-c6c([2H])c([2H])c([2H])c([2H])c6[2H])n5)cc4-n4c5ccccc5c5ccccc54)c([2H])c([2H])c([2H])c3c2c1[2H]]

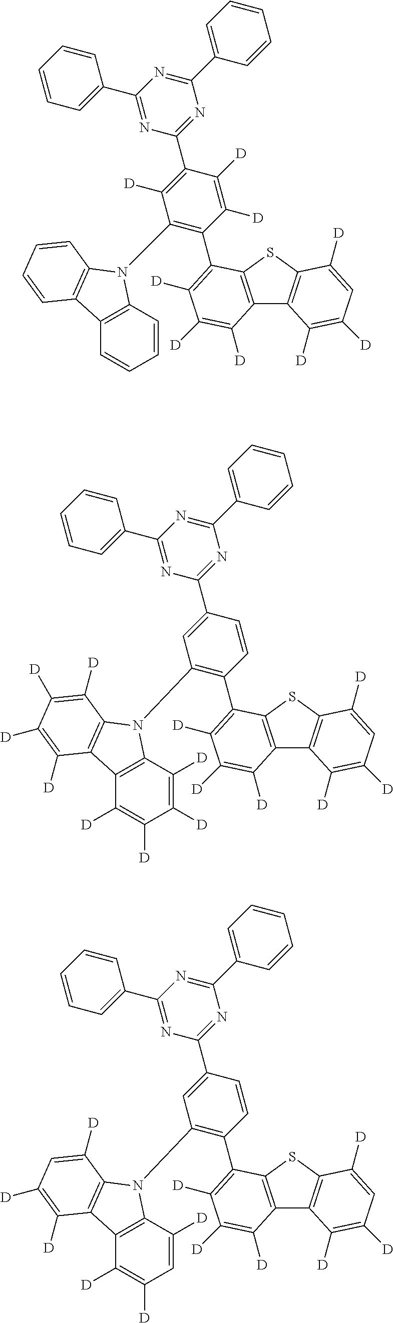 [SMILES: [2H]c1cc([2H])c2sc3c(-c4c([2H])c([2H])c(-c5nc(-c6ccccc6)nc(-c6ccccc6)n5)c([2H])c4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6ccccc6)n5)cc4-n4c5c([2H])c([2H])c([2H])c([2H])c5c5c([2H])c([2H])c([2H])c([2H])c54)c([2H])c([2H])c([2H])c3c2c1[2H] ; [2H]c1cc([2H])c2sc3c(-c4ccc(-c5nc(-c6ccccc6)nc(-c6ccccc6)n5)cc4-n4c5c([2H])cc([2H])c([2H])c5c5c([2H])c([2H])cc([2H])c54)c([2H])c([2H])c([2H])c3c2c1[2H]]

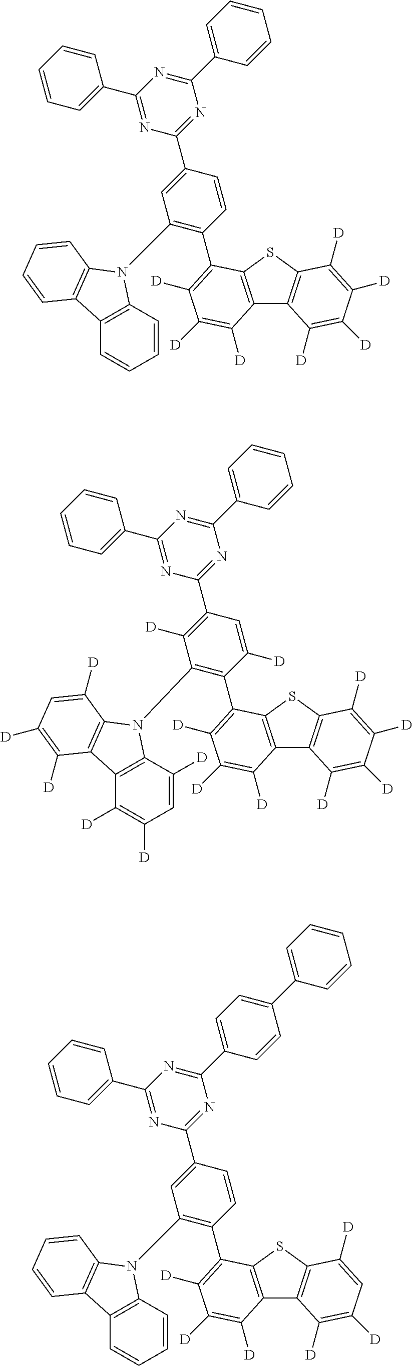 [SMILES: [2H]c1c([2H])c([2H])c2c(sc3c(-c4ccc(-c5nc(-c6ccccc6)nc(-c6ccccc6)n5)cc4-n4c5ccccc5c5ccccc54)c([2H])c([2H])c([2H])c32)c1[2H] ; [2H]c1cc(-c2nc(-c3ccccc3)nc(-c3ccccc3)n2)c([2H])c(-n2c3c([2H])cc([2H])c([2H])c3c3c([2H])c([2H])cc([2H])c32)c1-c1c([2H])c([2H])c([2H])c2c1sc1c([2H])c([2H])c([2H])c([2H])c12 ; [2H]c1cc([2H])c2sc3c(-c4ccc(-c5nc(-c6ccccc6)nc(-c6ccc(-c7ccccc7)cc6)n5)cc4-n4c5ccccc5c5ccccc54)c([2H])c([2H])c([2H])c3c2c1[2H]]

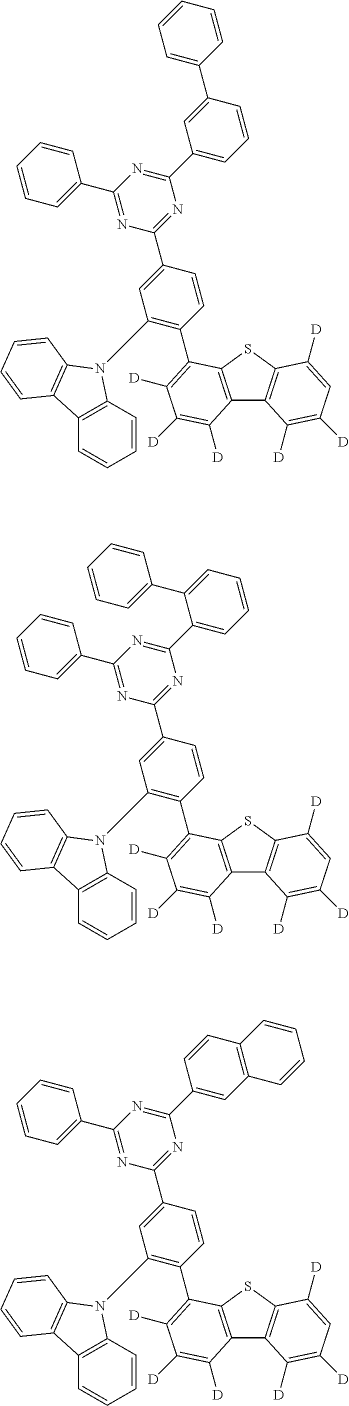 [SMILES: [2H]c1cc([2H])c2sc3c(-c4ccc(-c5nc(-c6ccccc6)nc(-c6cccc(-c7ccccc7)c6)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6ccccc6-c6ccccc6)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6ccc7ccccc7c6)n5)cc4-n4c5ccccc5c5ccccc54)c([2H])c([2H])c([2H])c3c2c1[2H]]

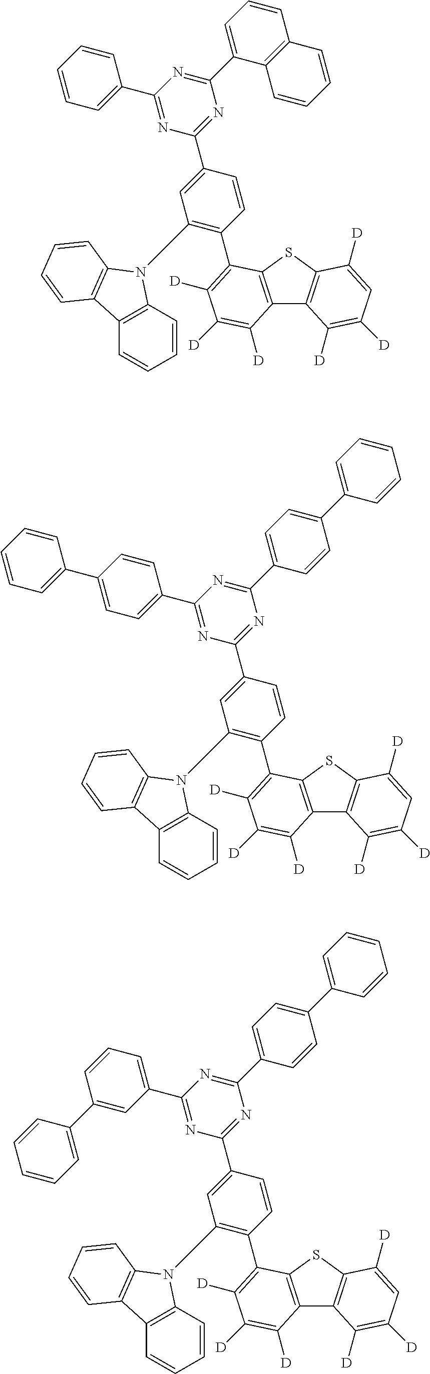 [SMILES: [2H]c1cc([2H])c2sc3c(-c4ccc(-c5nc(-c6ccccc6)nc(-c6cccc7ccccc67)n5)cc4-n4c5ccccc5c5ccccc54)c([2H])c([2H])c([2H])c3c2c1[2H] ; [2H]c1cc([2H])c2sc3c(-c4ccc(-c5nc(-c6ccc(-c7ccccc7)cc6)nc(-c6ccc(-c7ccccc7)cc6)n5)cc4-n4c5ccccc5c5ccccc54)c([2H])c([2H])c([2H])c3c2c1[2H] ; [2H]c1cc([2H])c2sc3c(-c4ccc(-c5nc(-c6ccc(-c7ccccc7)cc6)nc(-c6cccc(-c7ccccc7)c6)n5)cc4-n4c5ccccc5c5ccccc54)c([2H])c([2H])c([2H])c3c2c1[2H]]

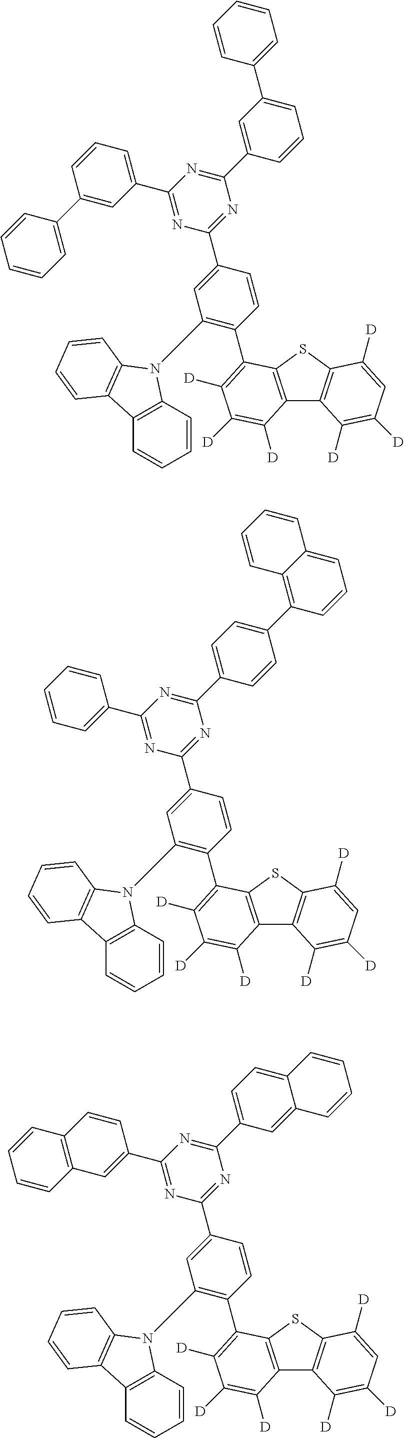 [SMILES: [2H]c1cc([2H])c2sc3c(-c4ccc(-c5nc(-c6cccc(-c7ccccc7)c6)nc(-c6cccc(-c7ccccc7)c6)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6ccc(-c7cccc8ccccc78)cc6)n5)cc4-n4c5ccccc5c5ccccc54)c([2H])c([2H])c([2H])c3c2c1[2H] ; [2H]c1cc([2H])c2sc3c(-c4ccc(-c5nc(-c6ccc7ccccc7c6)nc(-c6ccc7ccccc7c6)n5)cc4-n4c5ccccc5c5ccccc54)c([2H])c([2H])c([2H])c3c2c1[2H]]

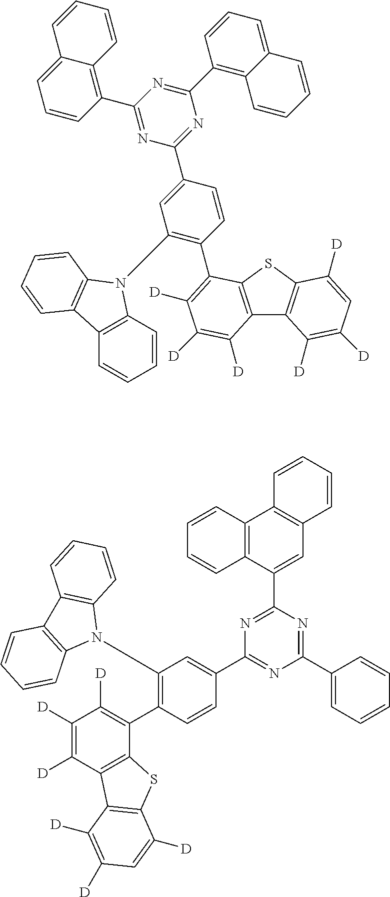 [SMILES: [2H]c1cc([2H])c2sc3c(-c4ccc(-c5nc(-c6cccc7ccccc67)nc(-c6cccc7ccccc67)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6cc7ccccc7c7ccccc67)n5)cc4-n4c5ccccc5c5ccccc54)c([2H])c([2H])c([2H])c3c2c1[2H]]

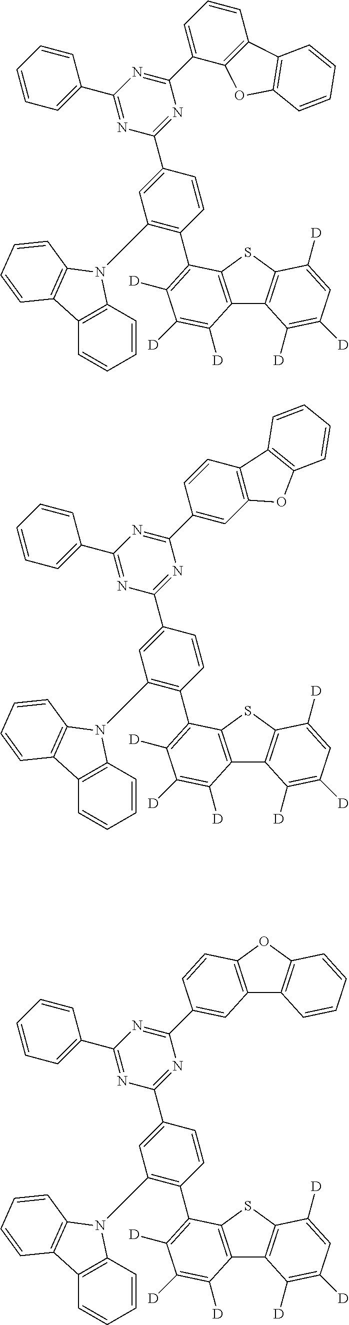 [SMILES: [2H]c1cc([2H])c2sc3c(-c4ccc(-c5nc(-c6ccccc6)nc(-c6cccc7c6oc6ccccc67)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6ccc7c(c6)oc6ccccc67)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6ccc7oc8ccccc8c7c6)n5)cc4-n4c5ccccc5c5ccccc54)c([2H])c([2H])c([2H])c3c2c1[2H]]

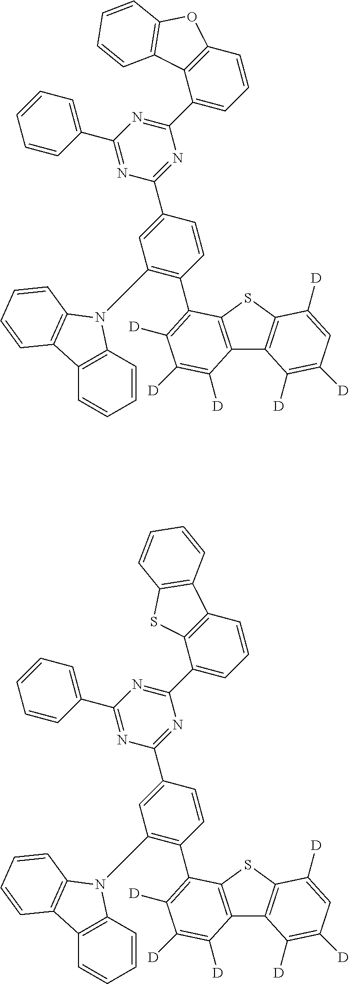 [SMILES: [2H]c1cc([2H])c2sc3c(-c4ccc(-c5nc(-c6ccccc6)nc(-c6cccc7oc8ccccc8c67)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6cccc7c6sc6ccccc67)n5)cc4-n4c5ccccc5c5ccccc54)c([2H])c([2H])c([2H])c3c2c1[2H]]

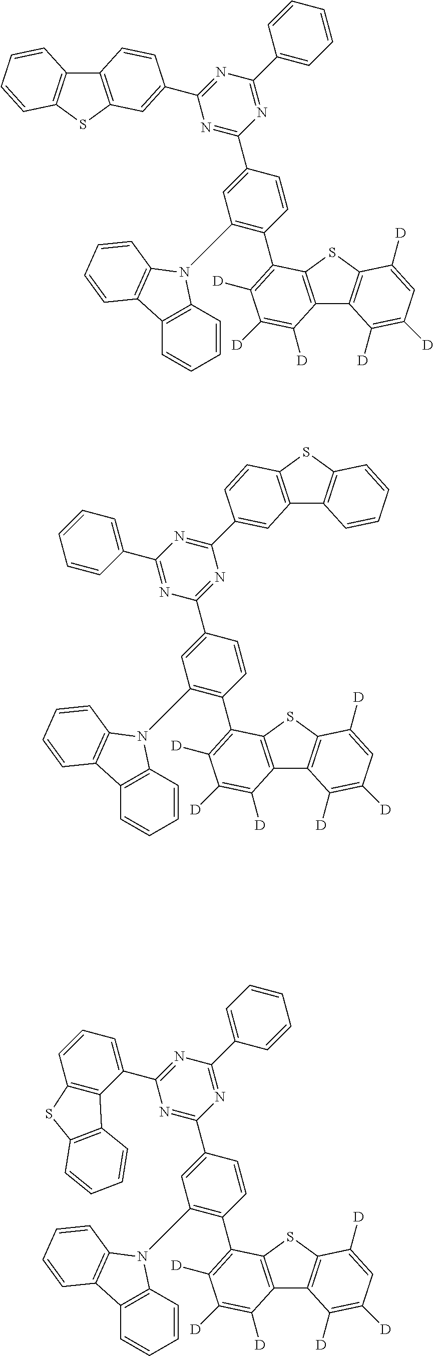 [SMILES: [2H]c1cc([2H])c2sc3c(-c4ccc(-c5nc(-c6ccccc6)nc(-c6ccc7c(c6)sc6ccccc67)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6ccc7sc8ccccc8c7c6)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6cccc7sc8ccccc8c67)n5)cc4-n4c5ccccc5c5ccccc54)c([2H])c([2H])c([2H])c3c2c1[2H]]

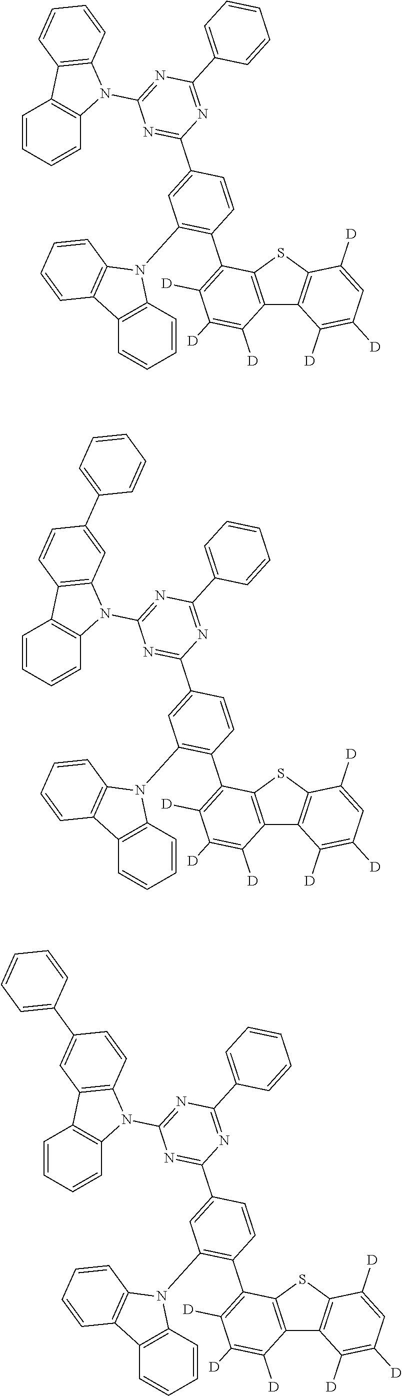 [SMILES: [2H]c1cc([2H])c2sc3c(-c4ccc(-c5nc(-c6ccccc6)nc(-n6c7ccccc7c7ccccc76)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-n6c7ccccc7c7ccc(-c8ccccc8)cc76)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-n6c7ccccc7c7cc(-c8ccccc8)ccc76)n5)cc4-n4c5ccccc5c5ccccc54)c([2H])c([2H])c([2H])c3c2c1[2H]]

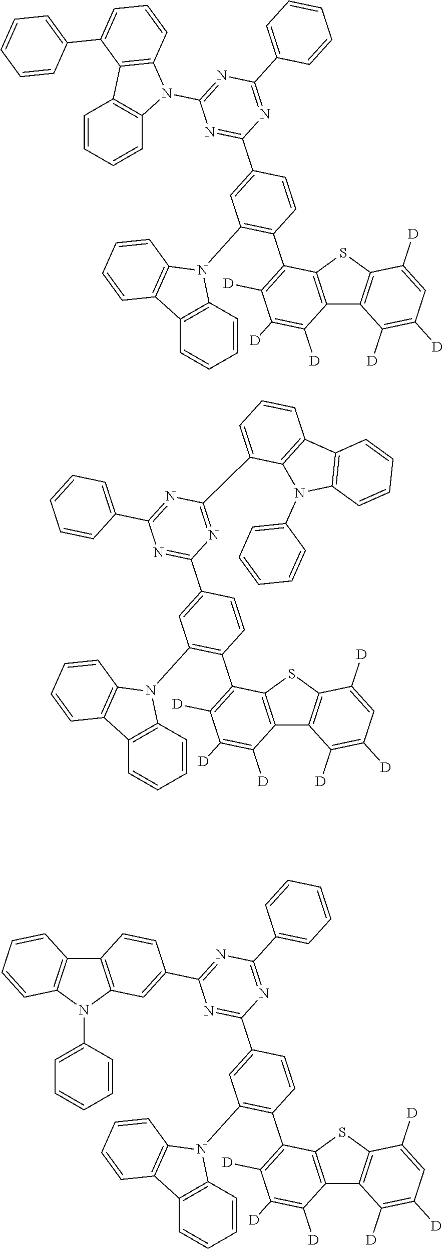 [SMILES: [2H]c1cc([2H])c2sc3c(-c4ccc(-c5nc(-c6ccccc6)nc(-n6c7ccccc7c7c(-c8ccccc8)cccc76)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6cccc7c8ccccc8n(-c8ccccc8)c67)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6ccc7c8ccccc8n(-c8ccccc8)c7c6)n5)cc4-n4c5ccccc5c5ccccc54)c([2H])c([2H])c([2H])c3c2c1[2H]]

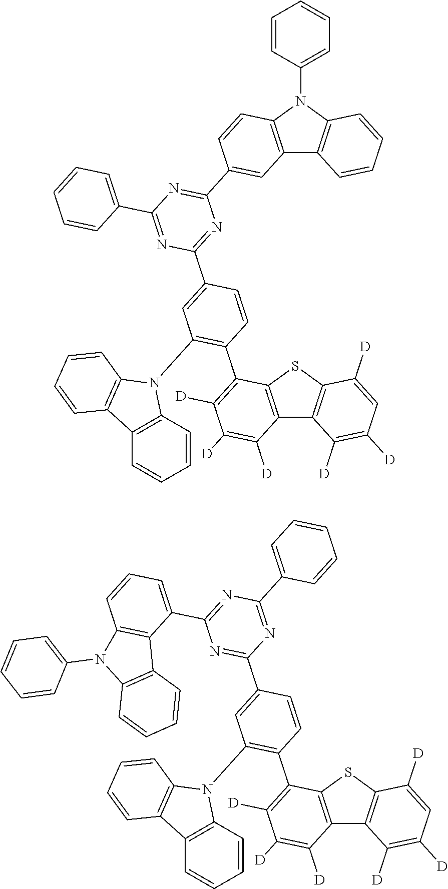 [SMILES: [2H]c1cc([2H])c2sc3c(-c4ccc(-c5nc(-c6ccccc6)nc(-c6ccc7c(c6)c6ccccc6n7-c6ccccc6)n5)cc4-n4c5ccccc5c5ccccc54)c([2H])c([2H])c([2H])c3c2c1[2H] ; [2H]c1cc([2H])c2sc3c(-c4ccc(-c5nc(-c6ccccc6)nc(-c6cccc7c6c6ccccc6n7-c6ccccc6)n5)cc4-n4c5ccccc5c5ccccc54)c([2H])c([2H])c([2H])c3c2c1[2H]]

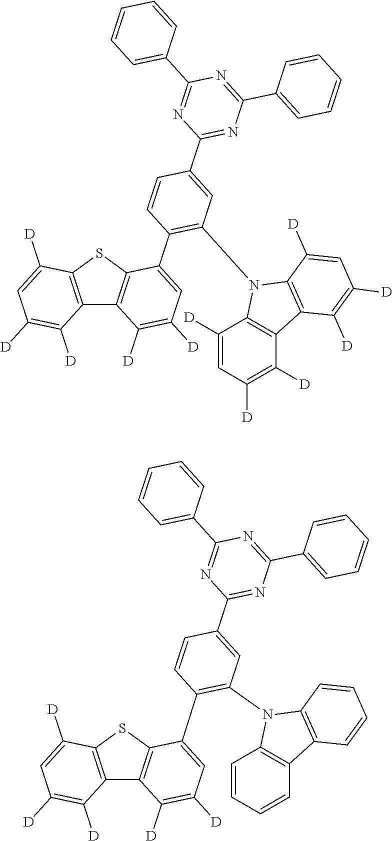 [SMILES: [2H]c1cc([2H])c2sc3c(-c4ccc(-c5nc(-c6ccccc6)nc(-c6ccccc6)n5)cc4-n4c5c([2H])cc([2H])c([2H])c5c5c([2H])c([2H])cc([2H])c54)cc([2H])c([2H])c3c2c1[2H] ; [2H]c1cc([2H])c2sc3c(-c4ccc(-c5nc(-c6ccccc6)nc(-c6ccccc6)n5)cc4-n4c5ccccc5c5ccccc54)cc([2H])c([2H])c3c2c1[2H]]

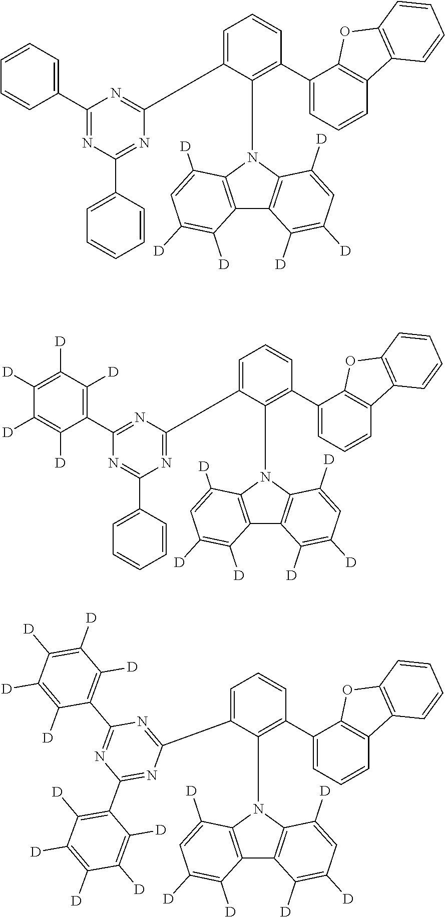 [SMILES: [2H]c1cc([2H])c2c(c1[2H])c1c([2H])c([2H])cc([2H])c1n2-c1c(-c2nc(-c3ccccc3)nc(-c3ccccc3)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3c([2H])c([2H])c([2H])c([2H])c3[2H])n2)cccc1-c1cccc2c1oc1ccccc12 ; [2H]c1cc([2H])c2c(c1[2H])c1c([2H])c([2H])cc([2H])c1n2-c1c(-c2nc(-c3c([2H])c([2H])c([2H])c([2H])c3[2H])nc(-c3c([2H])c([2H])c([2H])c([2H])c3[2H])n2)cccc1-c1cccc2c1oc1ccccc12]

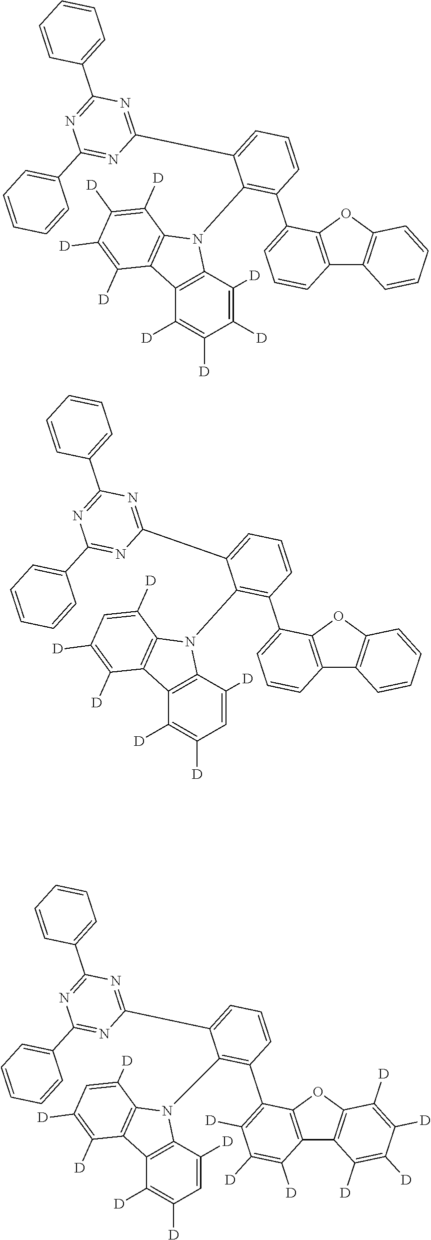 [SMILES: [2H]c1c([2H])c([2H])c2c(c1[2H])c1c([2H])c([2H])c([2H])c([2H])c1n2-c1c(-c2nc(-c3ccccc3)nc(-c3ccccc3)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3ccccc3)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3ccccc3)n2)cccc1-c1c([2H])c([2H])c([2H])c2c1oc1c([2H])c([2H])c([2H])c([2H])c12]

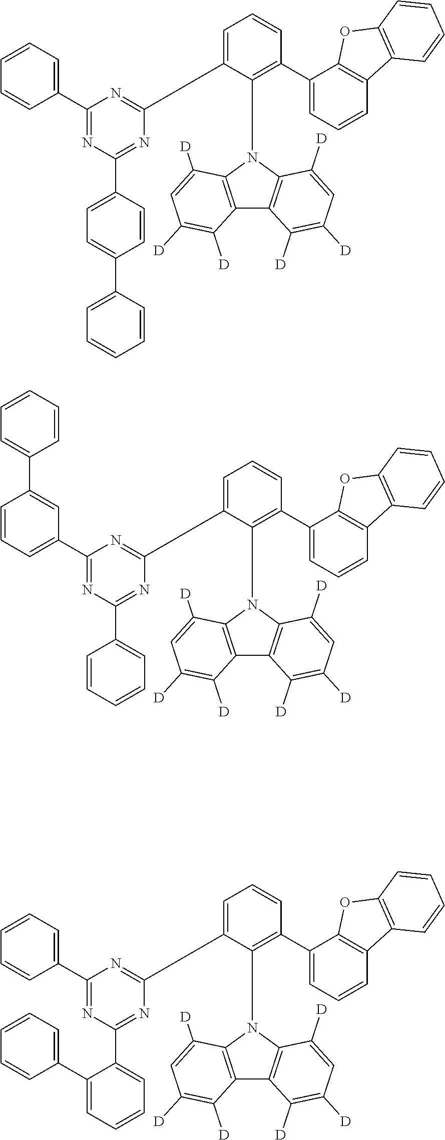 [SMILES: [2H]c1cc([2H])c2c(c1[2H])c1c([2H])c([2H])cc([2H])c1n2-c1c(-c2nc(-c3ccccc3)nc(-c3ccc(-c4ccccc4)cc3)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3cccc(-c4ccccc4)c3)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3ccccc3-c3ccccc3)n2)cccc1-c1cccc2c1oc1ccccc12]

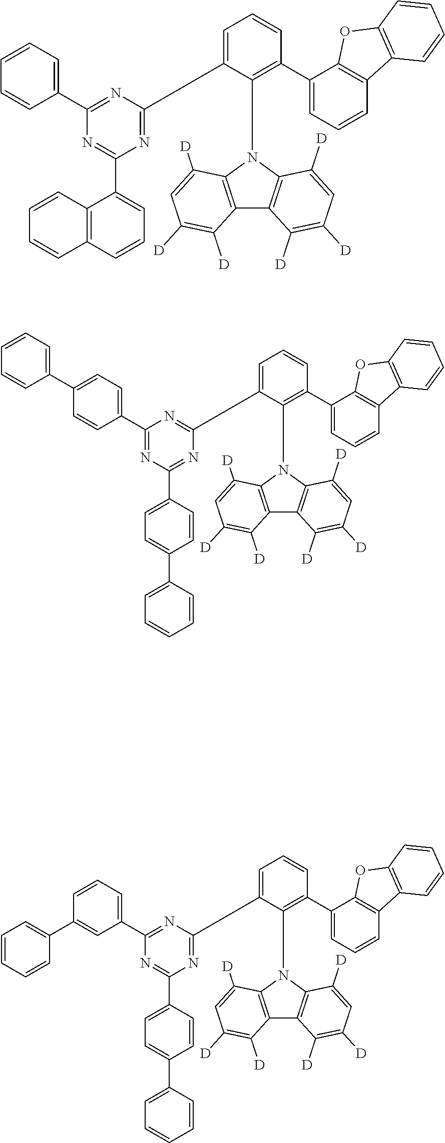 [SMILES: [2H]c1cc([2H])c2c(c1[2H])c1c([2H])c([2H])cc([2H])c1n2-c1c(-c2nc(-c3ccccc3)nc(-c3cccc4ccccc34)n2)cccc1-c1cccc2c1oc1ccccc12 ; [2H]c1cc([2H])c2c(c1[2H])c1c([2H])c([2H])cc([2H])c1n2-c1c(-c2nc(-c3ccc(-c4ccccc4)cc3)nc(-c3ccc(-c4ccccc4)cc3)n2)cccc1-c1cccc2c1oc1ccccc12 ; [2H]c1cc([2H])c2c(c1[2H])c1c([2H])c([2H])cc([2H])c1n2-c1c(-c2nc(-c3ccc(-c4ccccc4)cc3)nc(-c3cccc(-c4ccccc4)c3)n2)cccc1-c1cccc2c1oc1ccccc12]

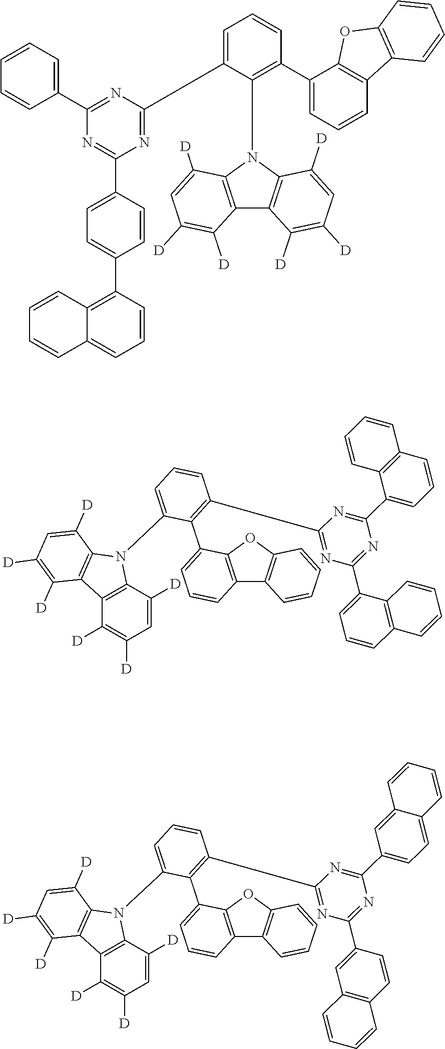 [SMILES: [2H]c1cc([2H])c2c(c1[2H])c1c([2H])c([2H])cc([2H])c1n2-c1c(-c2nc(-c3ccccc3)nc(-c3ccc(-c4cccc5ccccc45)cc3)n2)cccc1-c1cccc2c1oc1ccccc12 ; [2H]c1cc([2H])c2c(c1[2H])c1c([2H])c([2H])cc([2H])c1n2-c1cccc(-c2nc(-c3cccc4ccccc34)nc(-c3cccc4ccccc34)n2)c1-c1cccc2c1oc1ccccc12 ; [2H]c1cc([2H])c2c(c1[2H])c1c([2H])c([2H])cc([2H])c1n2-c1cccc(-c2nc(-c3ccc4ccccc4c3)nc(-c3ccc4ccccc4c3)n2)c1-c1cccc2c1oc1ccccc12]

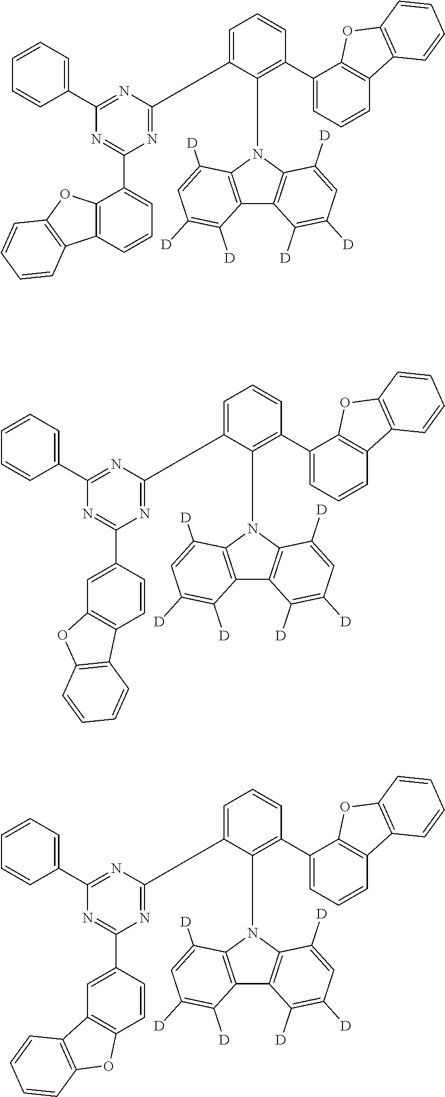 [SMILES: [2H]c1cc([2H])c2c(c1[2H])c1c([2H])c([2H])cc([2H])c1n2-c1c(-c2nc(-c3ccccc3)nc(-c3cccc4c3oc3ccccc34)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3ccc4c(c3)oc3ccccc34)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3ccc4oc5ccccc5c4c3)n2)cccc1-c1cccc2c1oc1ccccc12]

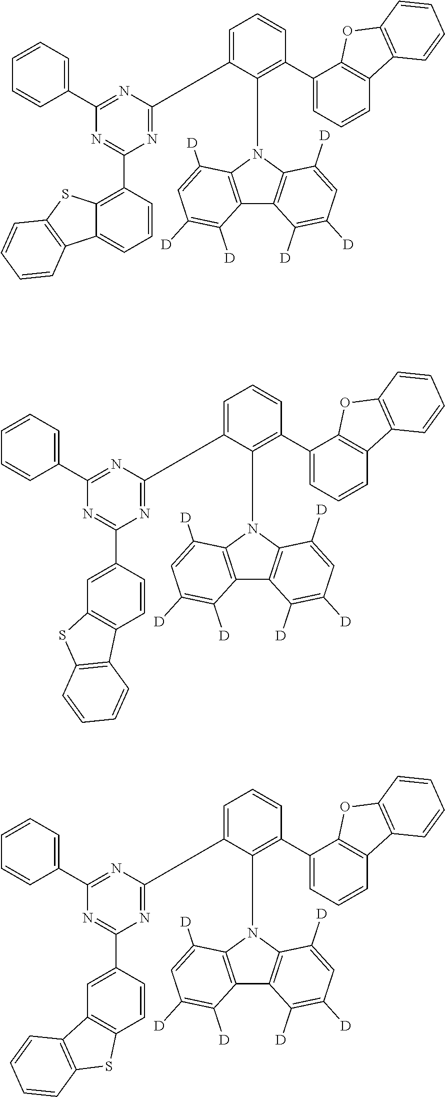 [SMILES: [2H]c1cc([2H])c2c(c1[2H])c1c([2H])c([2H])cc([2H])c1n2-c1c(-c2nc(-c3ccccc3)nc(-c3cccc4c3sc3ccccc34)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3ccc4c(c3)sc3ccccc34)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3ccc4sc5ccccc5c4c3)n2)cccc1-c1cccc2c1oc1ccccc12]

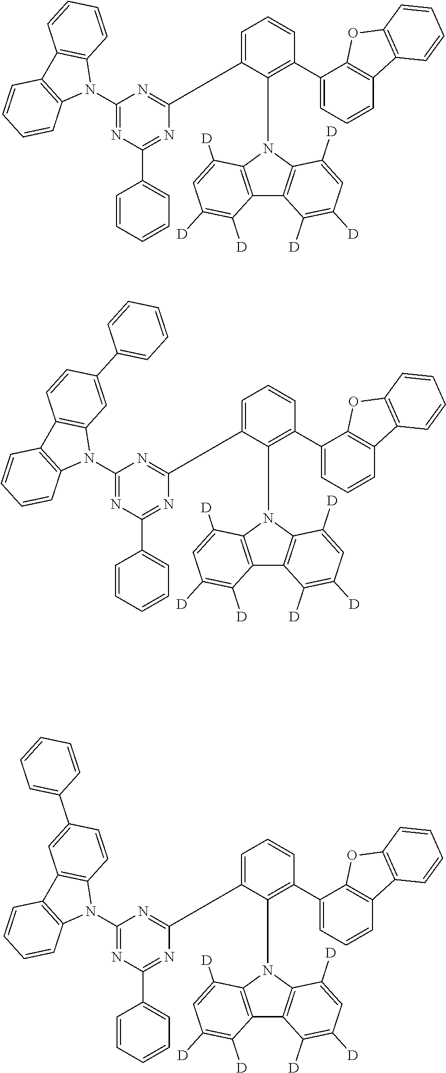 [SMILES: [2H]c1cc([2H])c2c(c1[2H])c1c([2H])c([2H])cc([2H])c1n2-c1c(-c2nc(-c3ccccc3)nc(-n3c4ccccc4c4ccccc43)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-n3c4ccccc4c4ccc(-c5ccccc5)cc43)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-n3c4ccccc4c4cc(-c5ccccc5)ccc43)n2)cccc1-c1cccc2c1oc1ccccc12]

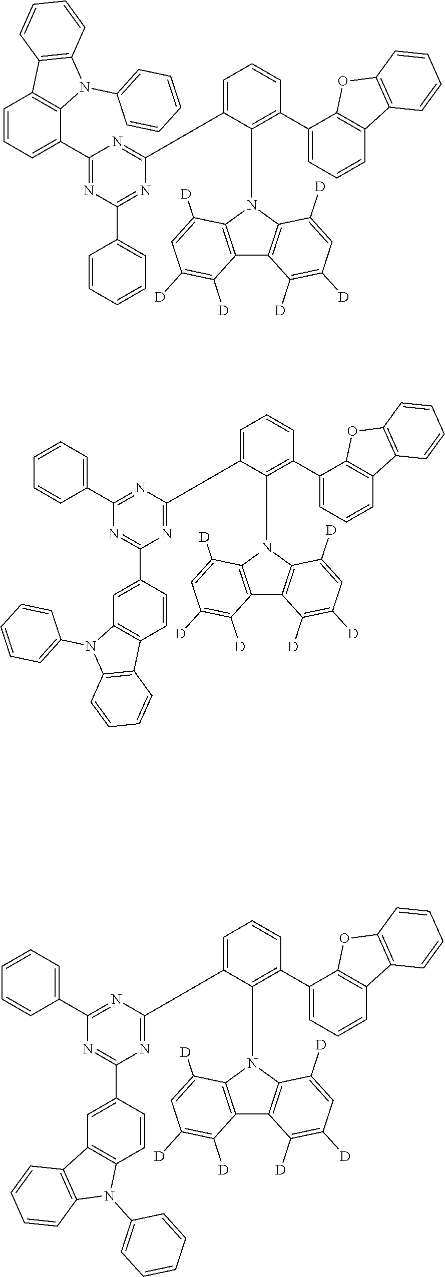 [SMILES: [2H]c1cc([2H])c2c(c1[2H])c1c([2H])c([2H])cc([2H])c1n2-c1c(-c2nc(-c3ccccc3)nc(-c3cccc4c5ccccc5n(-c5ccccc5)c34)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3ccc4c5ccccc5n(-c5ccccc5)c4c3)n2)cccc1-c1cccc2c1oc1ccccc12 ; [2H]c1cc([2H])c2c(c1[2H])c1c([2H])c([2H])cc([2H])c1n2-c1c(-c2nc(-c3ccccc3)nc(-c3ccc4c(c3)c3ccccc3n4-c3ccccc3)n2)cccc1-c1cccc2c1oc1ccccc12]

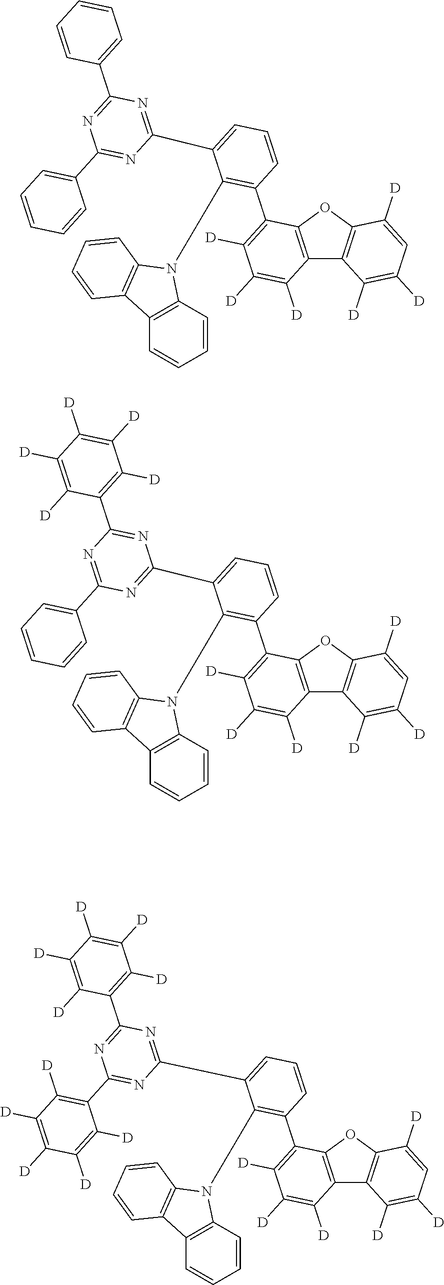 [SMILES: [2H]c1cc([2H])c2oc3c(-c4cccc(-c5nc(-c6ccccc6)nc(-c6ccccc6)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6c([2H])c([2H])c([2H])c([2H])c6[2H])n5)c4-n4c5ccccc5c5ccccc54)c([2H])c([2H])c([2H])c3c2c1[2H] ; [2H]c1cc([2H])c2oc3c(-c4cccc(-c5nc(-c6c([2H])c([2H])c([2H])c([2H])c6[2H])nc(-c6c([2H])c([2H])c([2H])c([2H])c6[2H])n5)c4-n4c5ccccc5c5ccccc54)c([2H])c([2H])c([2H])c3c2c1[2H]]

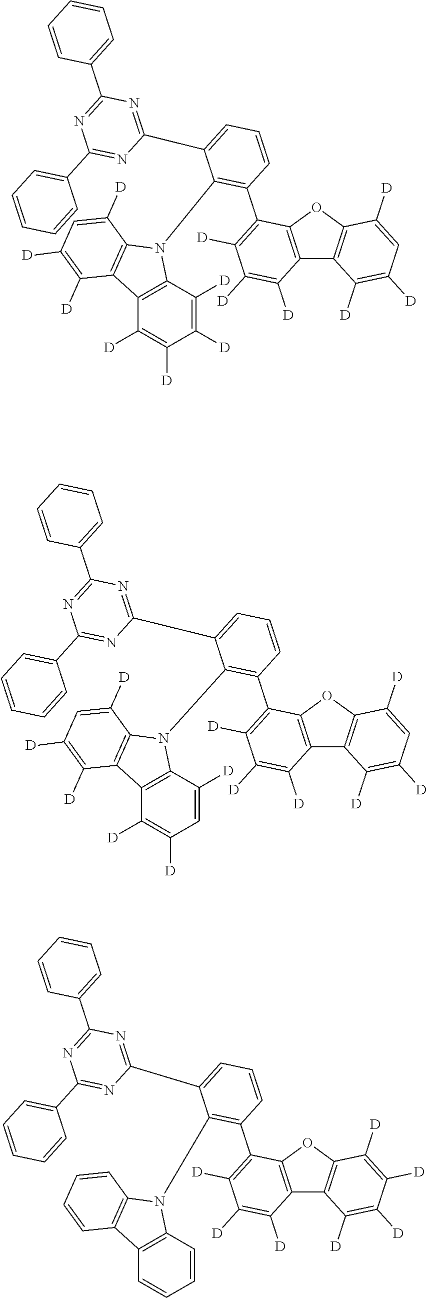 [SMILES: [2H]c1cc([2H])c2oc3c(-c4cccc(-c5nc(-c6ccccc6)nc(-c6ccccc6)n5)c4-n4c5c([2H])cc([2H])c([2H])c5c5c([2H])c([2H])c([2H])c([2H])c54)c([2H])c([2H])c([2H])c3c2c1[2H] ; [2H]c1cc([2H])c2oc3c(-c4cccc(-c5nc(-c6ccccc6)nc(-c6ccccc6)n5)c4-n4c5c([2H])cc([2H])c([2H])c5c5c([2H])c([2H])cc([2H])c54)c([2H])c([2H])c([2H])c3c2c1[2H] ; [2H]c1c([2H])c([2H])c2c(oc3c(-c4cccc(-c5nc(-c6ccccc6)nc(-c6ccccc6)n5)c4-n4c5ccccc5c5ccccc54)c([2H])c([2H])c([2H])c32)c1[2H]]

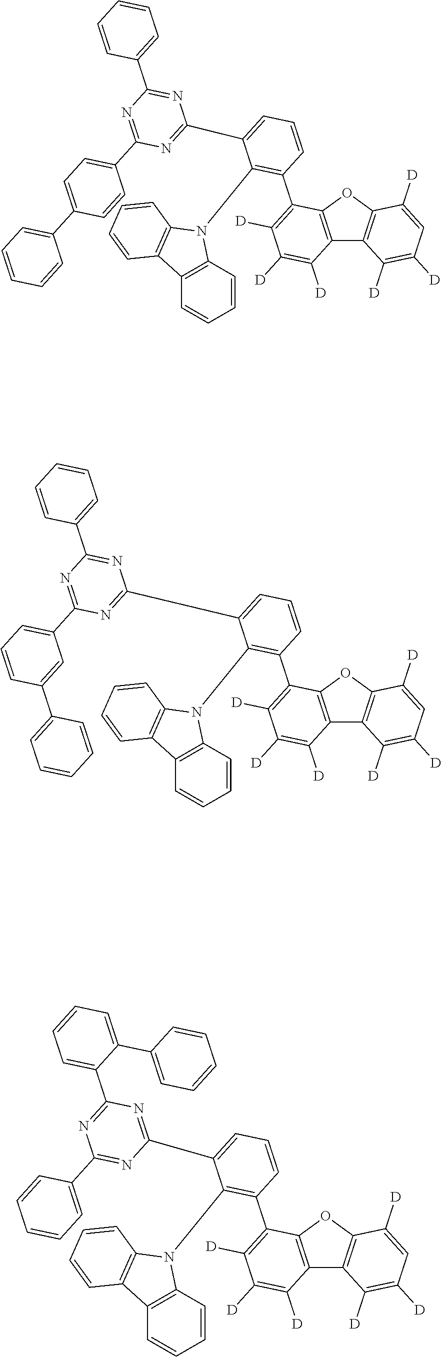 [SMILES: [2H]c1cc([2H])c2oc3c(-c4cccc(-c5nc(-c6ccccc6)nc(-c6ccc(-c7ccccc7)cc6)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6cccc(-c7ccccc7)c6)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6ccccc6-c6ccccc6)n5)c4-n4c5ccccc5c5ccccc54)c([2H])c([2H])c([2H])c3c2c1[2H]]

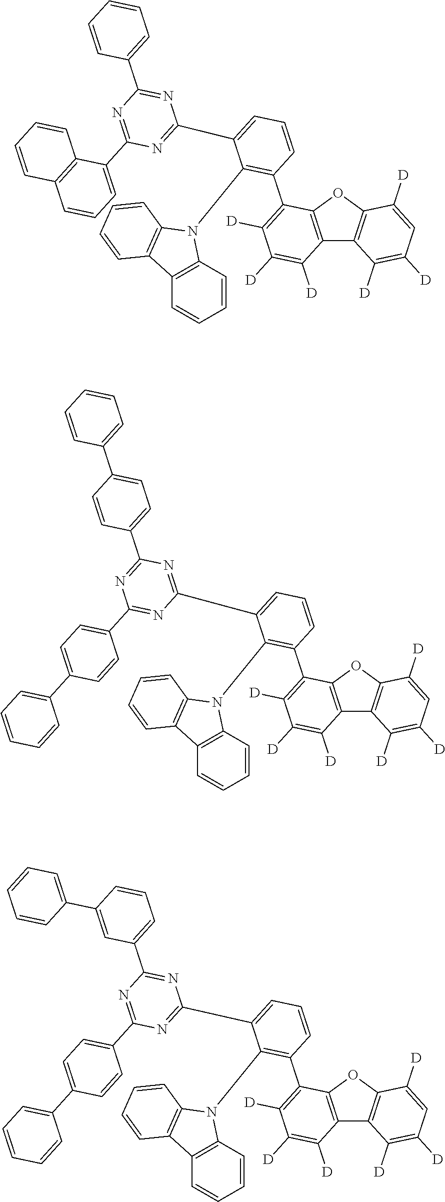 [SMILES: [2H]c1cc([2H])c2oc3c(-c4cccc(-c5nc(-c6ccccc6)nc(-c6cccc7ccccc67)n5)c4-n4c5ccccc5c5ccccc54)c([2H])c([2H])c([2H])c3c2c1[2H] ; [2H]c1cc([2H])c2oc3c(-c4cccc(-c5nc(-c6ccc(-c7ccccc7)cc6)nc(-c6ccc(-c7ccccc7)cc6)n5)c4-n4c5ccccc5c5ccccc54)c([2H])c([2H])c([2H])c3c2c1[2H] ; [2H]c1cc([2H])c2oc3c(-c4cccc(-c5nc(-c6ccc(-c7ccccc7)cc6)nc(-c6cccc(-c7ccccc7)c6)n5)c4-n4c5ccccc5c5ccccc54)c([2H])c([2H])c([2H])c3c2c1[2H]]

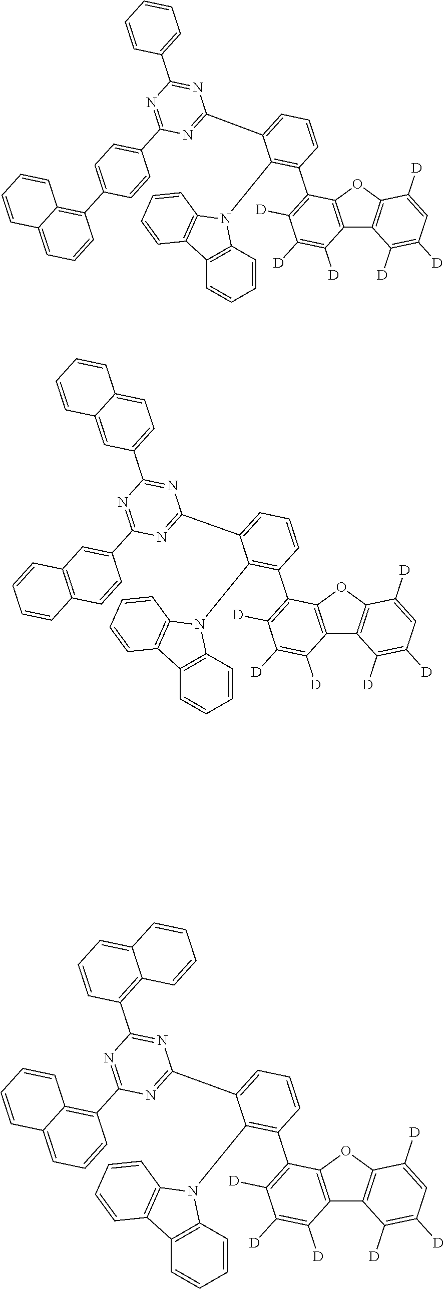 [SMILES: [2H]c1cc([2H])c2oc3c(-c4cccc(-c5nc(-c6ccccc6)nc(-c6ccc(-c7cccc8ccccc78)cc6)n5)c4-n4c5ccccc5c5ccccc54)c([2H])c([2H])c([2H])c3c2c1[2H] ; [2H]c1cc([2H])c2oc3c(-c4cccc(-c5nc(-c6ccc7ccccc7c6)nc(-c6ccc7ccccc7c6)n5)c4-n4c5ccccc5c5ccccc54)c([2H])c([2H])c([2H])c3c2c1[2H] ; [2H]c1cc([2H])c2oc3c(-c4cccc(-c5nc(-c6cccc7ccccc67)nc(-c6cccc7ccccc67)n5)c4-n4c5ccccc5c5ccccc54)c([2H])c([2H])c([2H])c3c2c1[2H]]

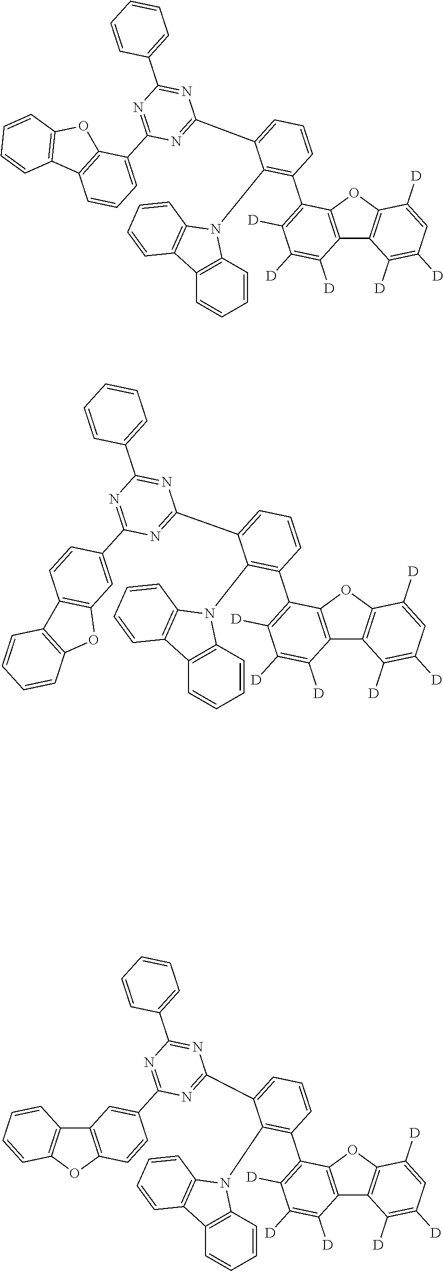 [SMILES: [2H]c1cc([2H])c2oc3c(-c4cccc(-c5nc(-c6ccccc6)nc(-c6cccc7c6oc6ccccc67)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6ccc7c(c6)oc6ccccc67)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6ccc7oc8ccccc8c7c6)n5)c4-n4c5ccccc5c5ccccc54)c([2H])c([2H])c([2H])c3c2c1[2H]]

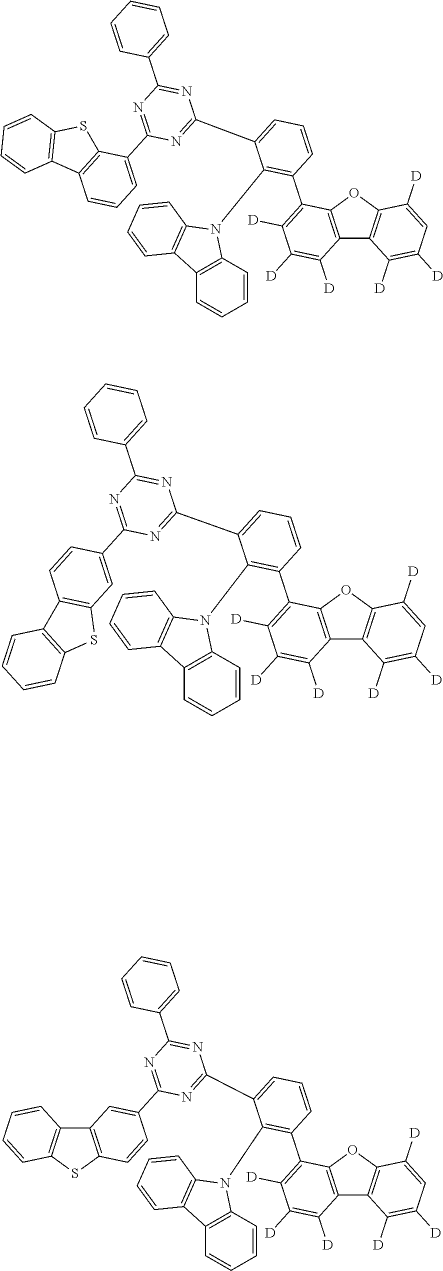 [SMILES: [2H]c1cc([2H])c2oc3c(-c4cccc(-c5nc(-c6ccccc6)nc(-c6cccc7c6sc6ccccc67)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6ccc7c(c6)sc6ccccc67)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6ccc7sc8ccccc8c7c6)n5)c4-n4c5ccccc5c5ccccc54)c([2H])c([2H])c([2H])c3c2c1[2H]]

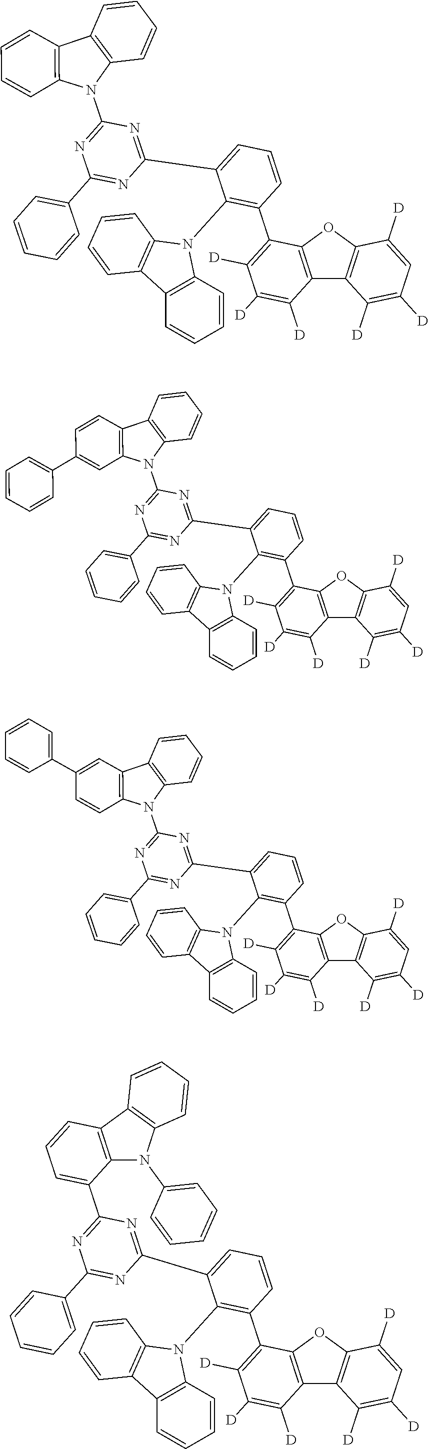

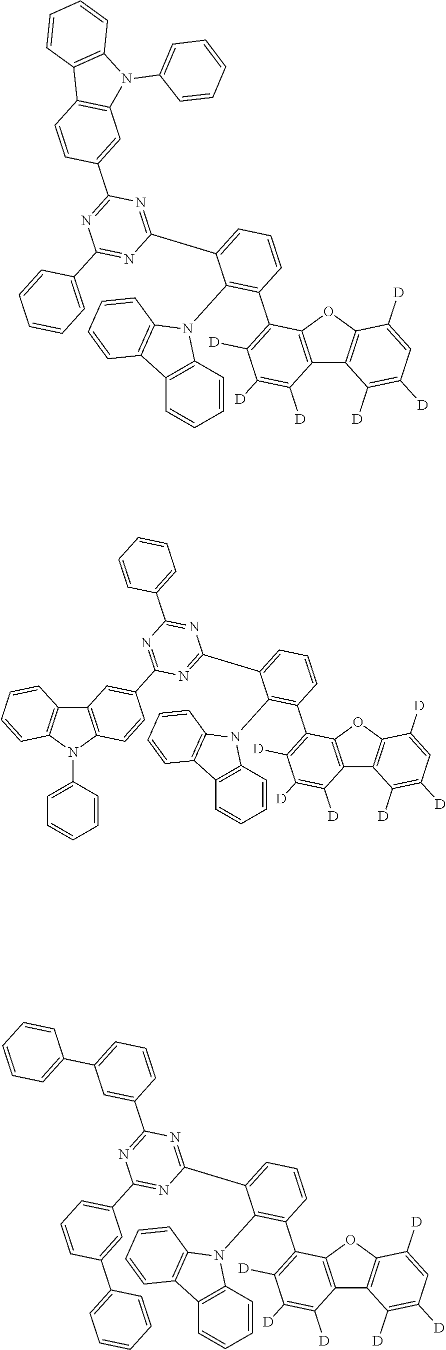 [SMILES: [2H]c1cc([2H])c2oc3c(-c4cccc(-c5nc(-c6ccccc6)nc(-c6ccc7c8ccccc8n(-c8ccccc8)c7c6)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6ccc7c(c6)c6ccccc6n7-c6ccccc6)n5)c4-n4c5ccccc5c5ccccc54)c([2H])c([2H])c([2H])c3c2c1[2H] ; [2H]c1cc([2H])c2oc3c(-c4cccc(-c5nc(-c6cccc(-c7ccccc7)c6)nc(-c6cccc(-c7ccccc7)c6)n5)c4-n4c5ccccc5c5ccccc54)c([2H])c([2H])c([2H])c3c2c1[2H]]

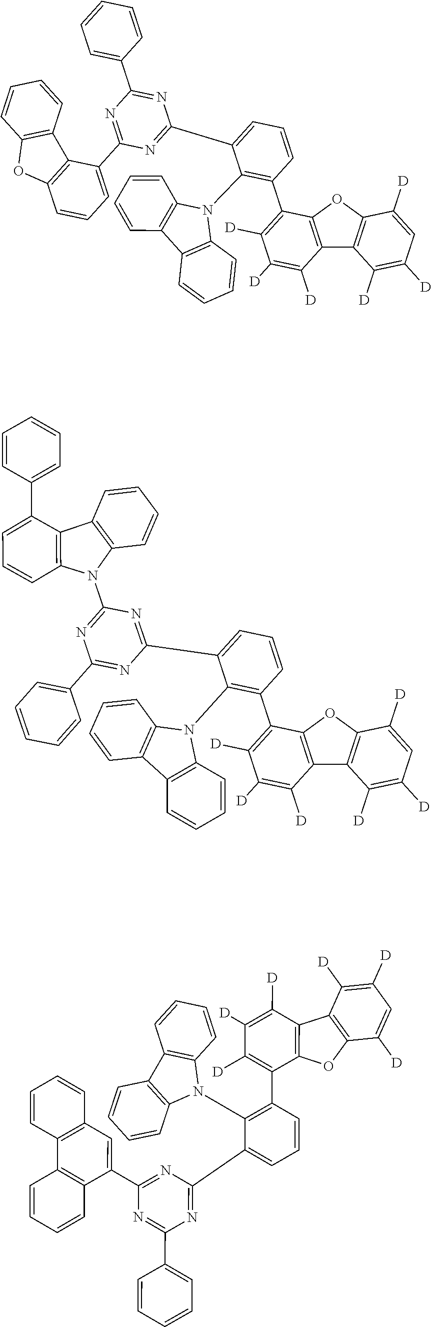 [SMILES: [2H]c1cc([2H])c2oc3c(-c4cccc(-c5nc(-c6ccccc6)nc(-c6cccc7oc8ccccc8c67)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-n6c7ccccc7c7c(-c8ccccc8)cccc76)n5)c4-n4c5ccccc5c5ccccc54)c([2H])c([2H])c([2H])c3c2c1[2H] ; [2H]c1cc([2H])c2oc3c(-c4cccc(-c5nc(-c6ccccc6)nc(-c6cc7ccccc7c7ccccc67)n5)c4-n4c5ccccc5c5ccccc54)c([2H])c([2H])c([2H])c3c2c1[2H]]

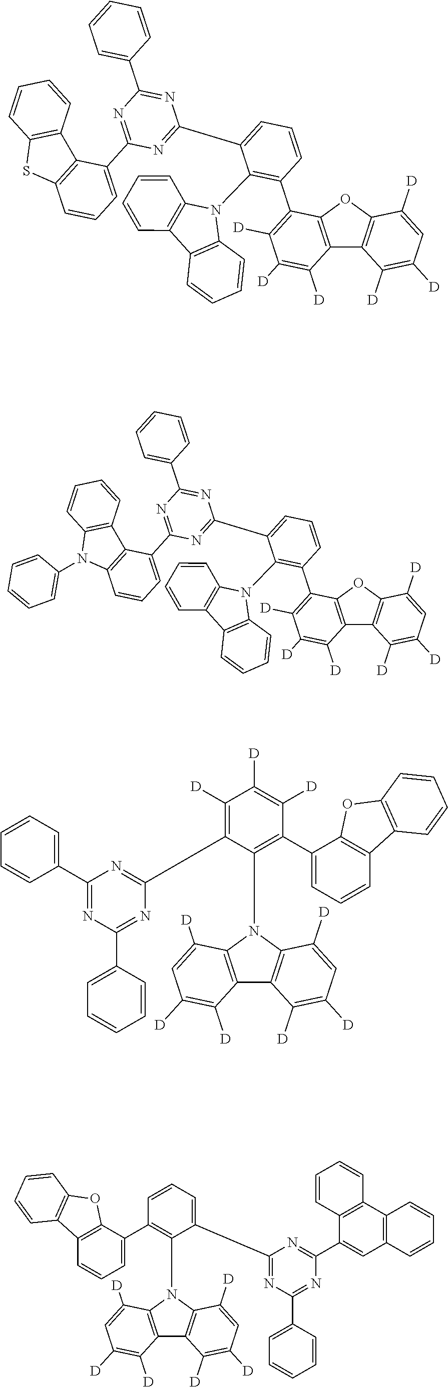

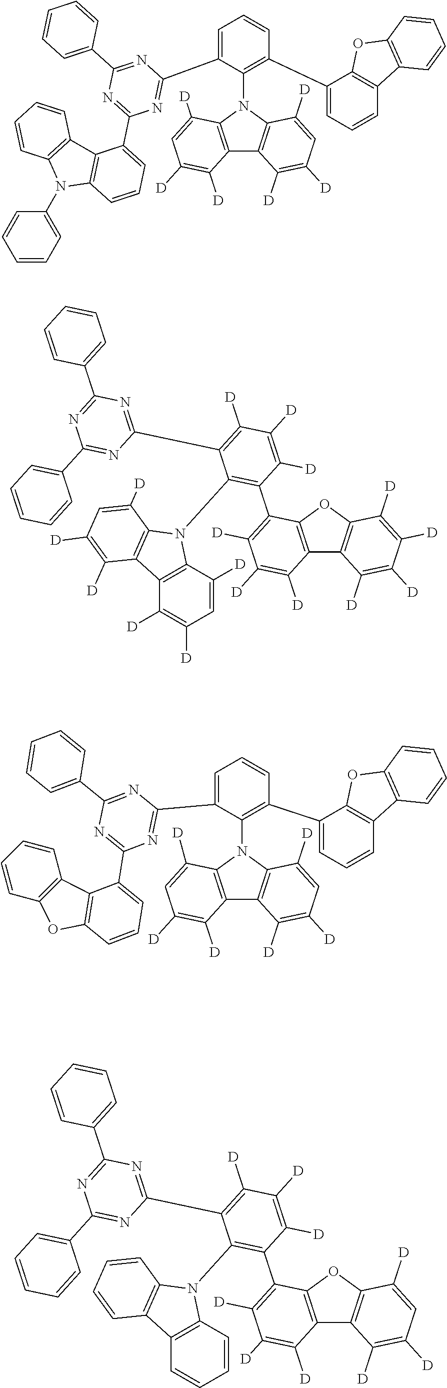

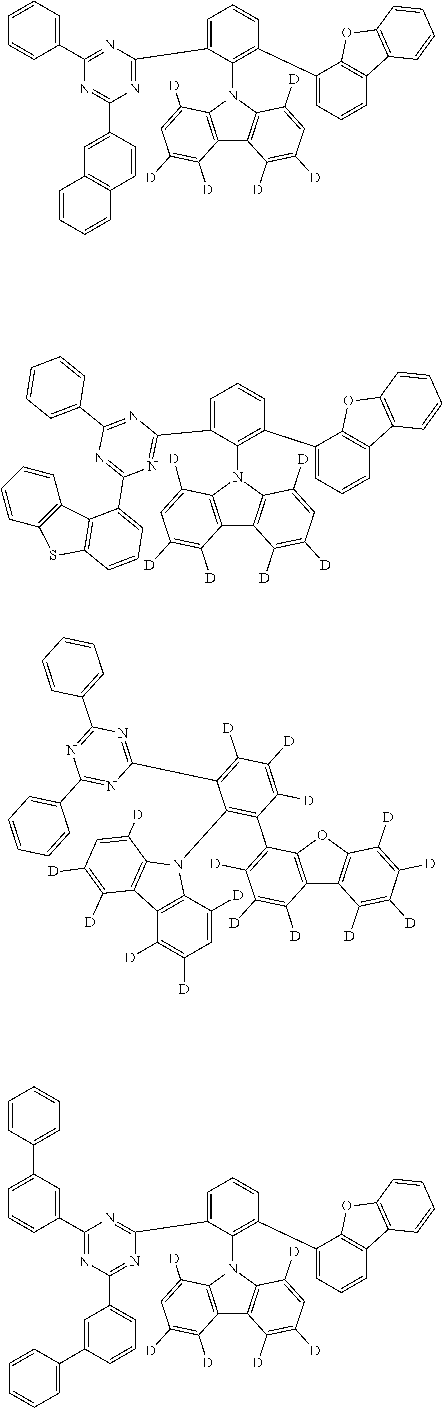

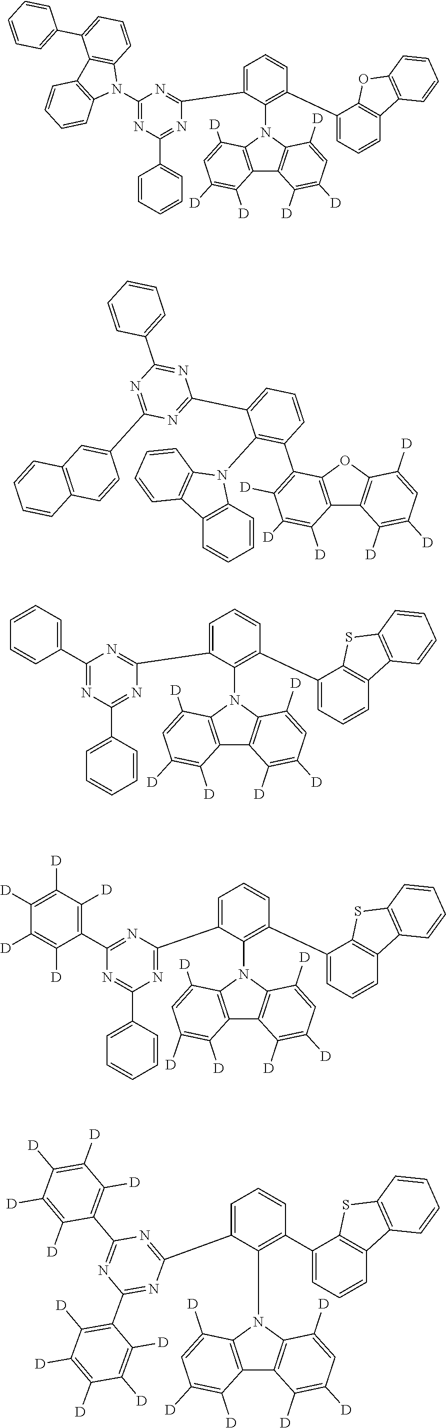

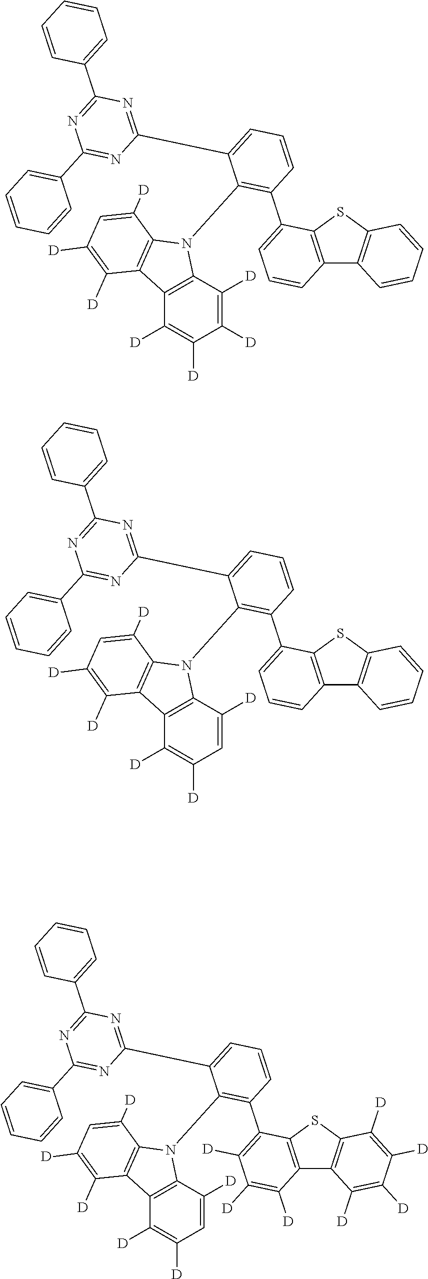 [SMILES: [2H]c1cc([2H])c2c(c1[2H])c1c([2H])c([2H])c([2H])c([2H])c1n2-c1c(-c2nc(-c3ccccc3)nc(-c3ccccc3)n2)cccc1-c1cccc2c1sc1ccccc12 ; [2H]c1cc([2H])c2c(c1[2H])c1c([2H])c([2H])cc([2H])c1n2-c1c(-c2nc(-c3ccccc3)nc(-c3ccccc3)n2)cccc1-c1cccc2c1sc1ccccc12 ; [2H]c1cc([2H])c2c(c1[2H])c1c([2H])c([2H])cc([2H])c1n2-c1c(-c2nc(-c3ccccc3)nc(-c3ccccc3)n2)cccc1-c1c([2H])c([2H])c([2H])c2c1sc1c([2H])c([2H])c([2H])c([2H])c12]

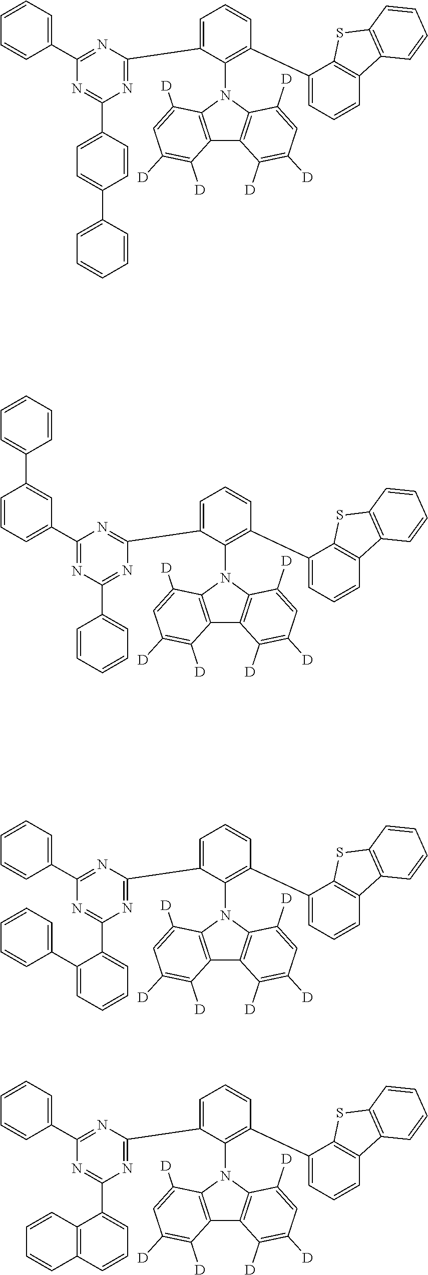

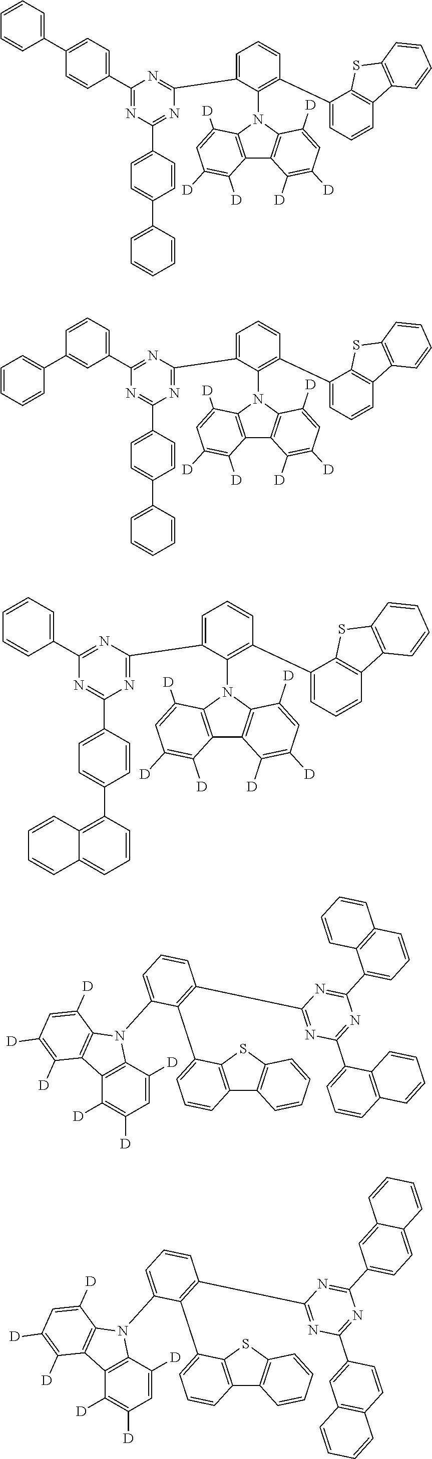

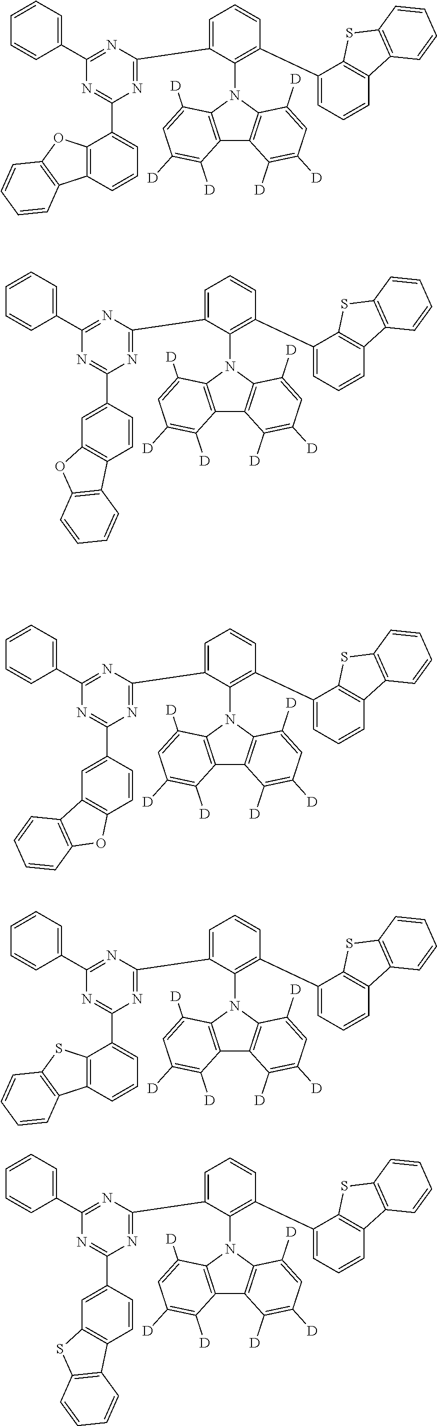

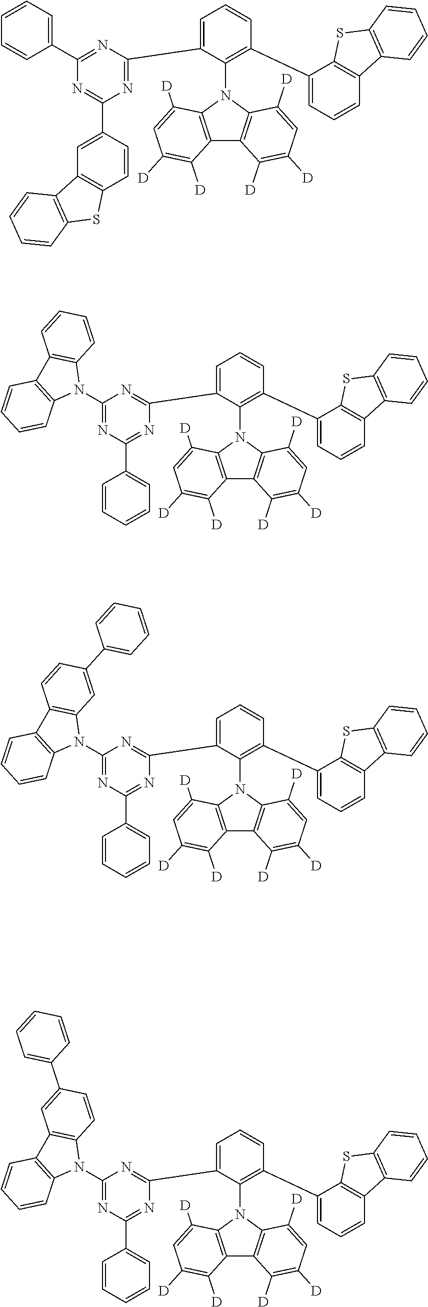

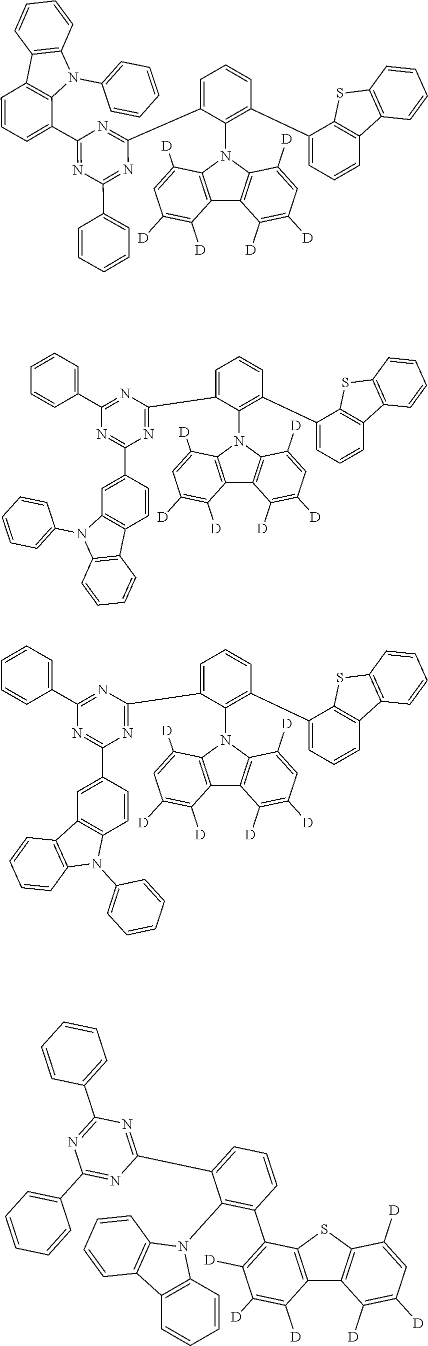

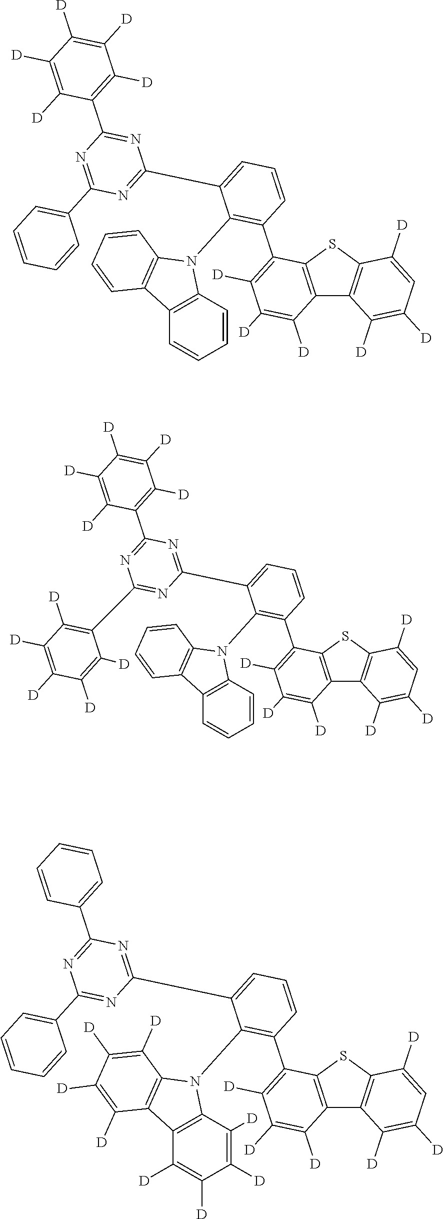 [SMILES: [2H]c1cc([2H])c2sc3c(-c4cccc(-c5nc(-c6ccccc6)nc(-c6c([2H])c([2H])c([2H])c([2H])c6[2H])n5)c4-n4c5ccccc5c5ccccc54)c([2H])c([2H])c([2H])c3c2c1[2H] ; [2H]c1cc([2H])c2sc3c(-c4cccc(-c5nc(-c6c([2H])c([2H])c([2H])c([2H])c6[2H])nc(-c6c([2H])c([2H])c([2H])c([2H])c6[2H])n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccccc6)n5)c4-n4c5c([2H])c([2H])c([2H])c([2H])c5c5c([2H])c([2H])c([2H])c([2H])c54)c([2H])c([2H])c([2H])c3c2c1[2H]]

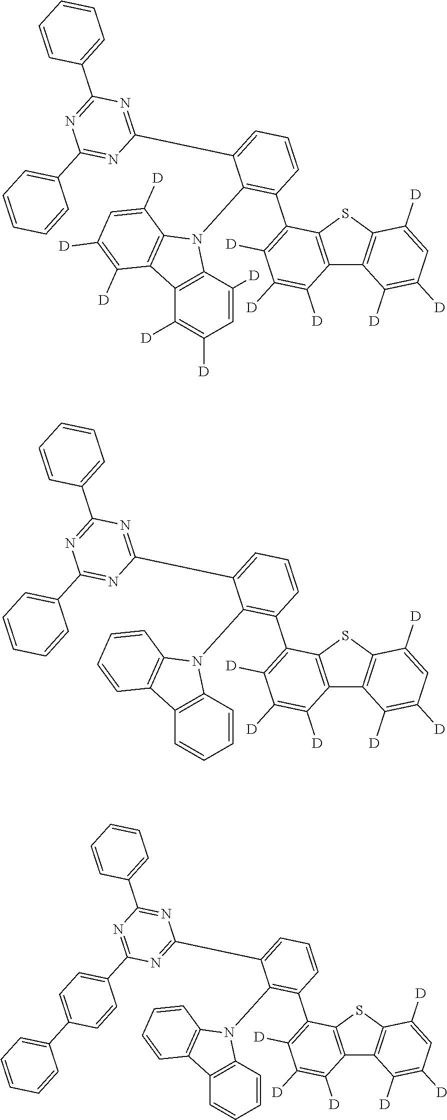 [SMILES: [2H]c1cc([2H])c2sc3c(-c4cccc(-c5nc(-c6ccccc6)nc(-c6ccccc6)n5)c4-n4c5c([2H])cc([2H])c([2H])c5c5c([2H])c([2H])cc([2H])c54)c([2H])c([2H])c([2H])c3c2c1[2H] ; [2H]c1cc([2H])c2sc3c(-c4cccc(-c5nc(-c6ccccc6)nc(-c6ccccc6)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccc(-c7ccccc7)cc6)n5)c4-n4c5ccccc5c5ccccc54)c([2H])c([2H])c([2H])c3c2c1[2H]]

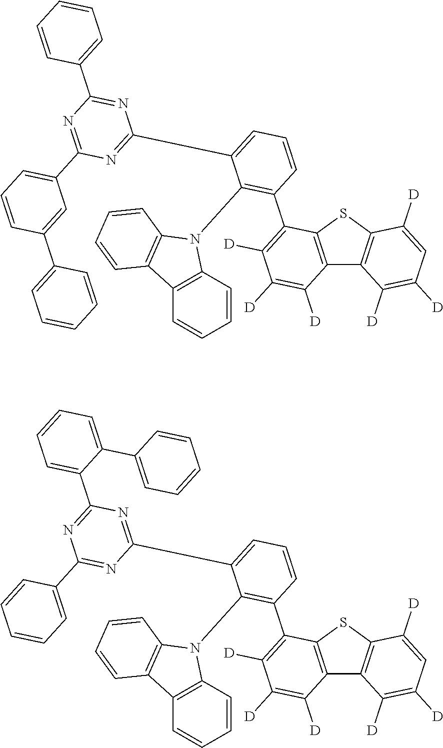 [SMILES: [2H]c1cc([2H])c2sc3c(-c4cccc(-c5nc(-c6ccccc6)nc(-c6cccc(-c7ccccc7)c6)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccccc6-c6ccccc6)n5)c4-n4c5ccccc5c5ccccc54)c([2H])c([2H])c([2H])c3c2c1[2H]]

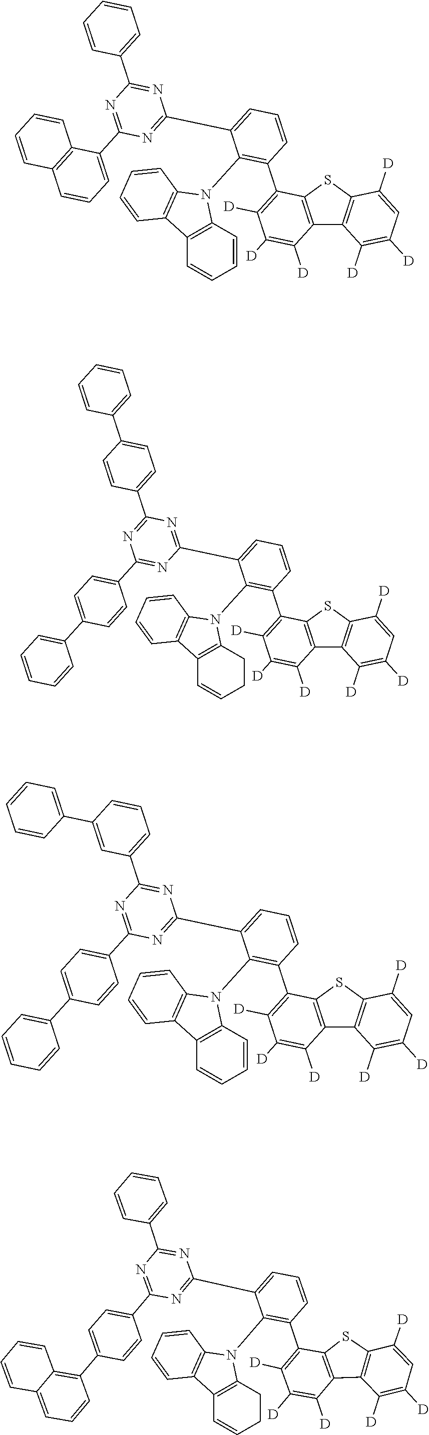

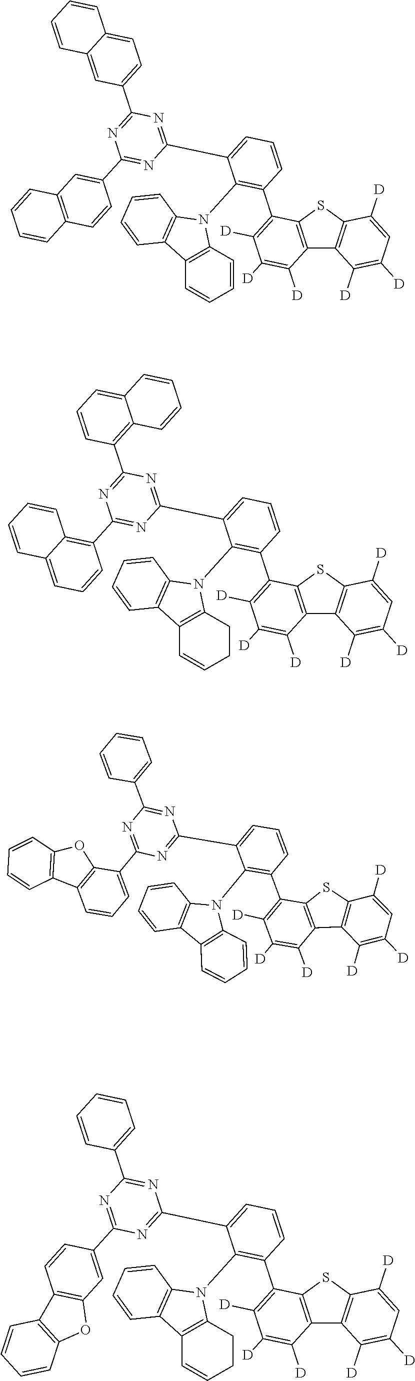

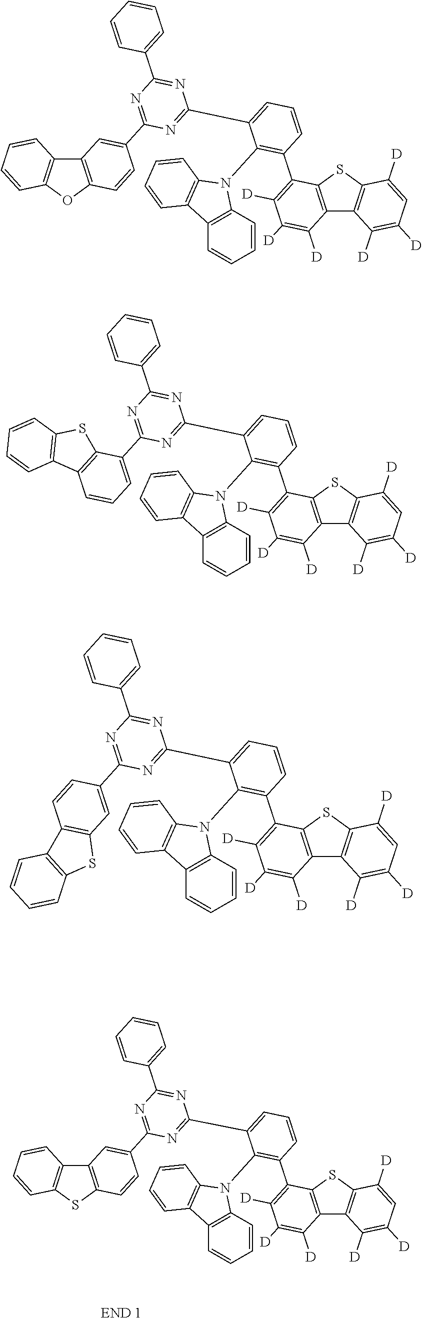

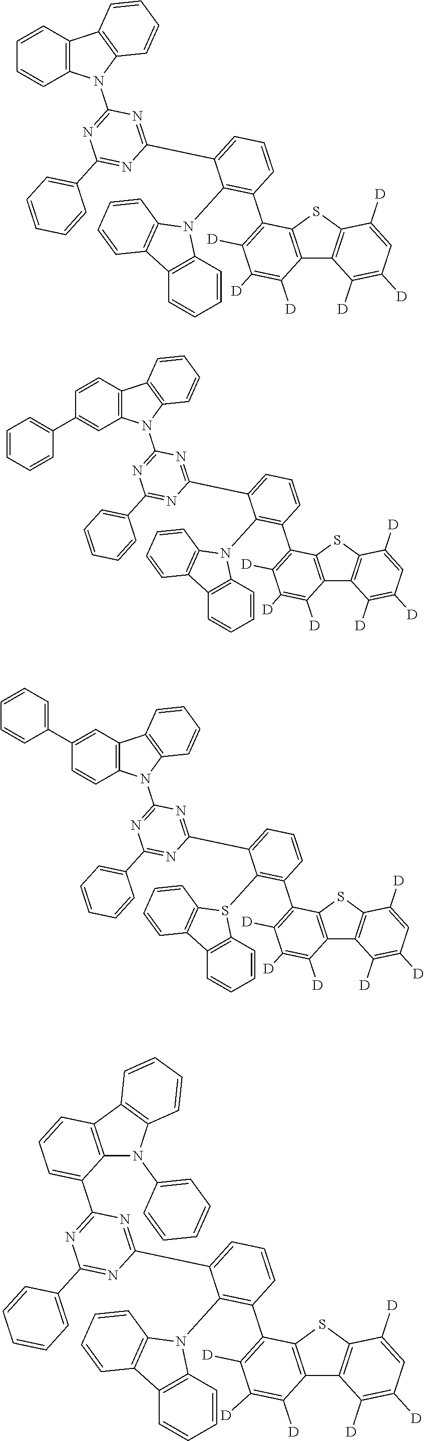

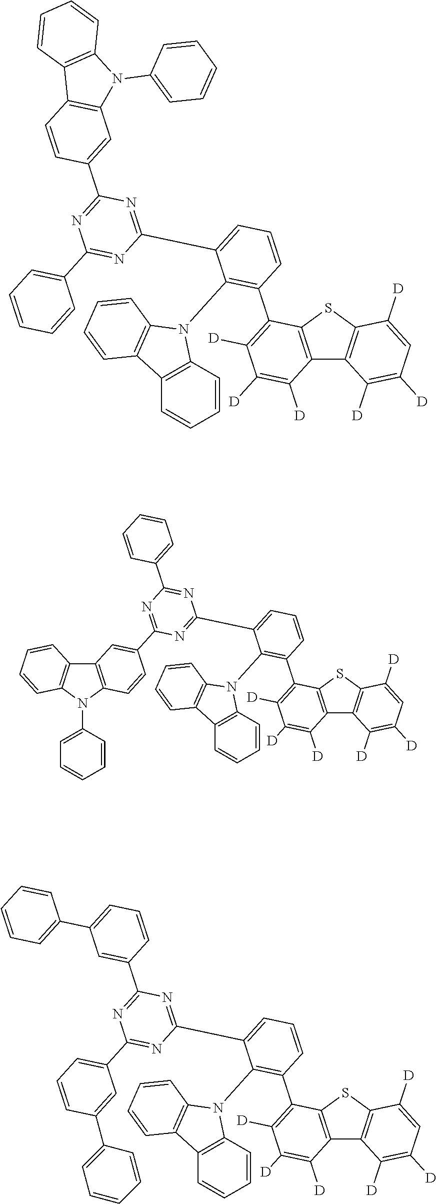 [SMILES: [2H]c1cc([2H])c2sc3c(-c4cccc(-c5nc(-c6ccccc6)nc(-c6ccc7c8ccccc8n(-c8ccccc8)c7c6)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6ccc7c(c6)c6ccccc6n7-c6ccccc6)n5)c4-n4c5ccccc5c5ccccc54)c([2H])c([2H])c([2H])c3c2c1[2H] ; [2H]c1cc([2H])c2sc3c(-c4cccc(-c5nc(-c6cccc(-c7ccccc7)c6)nc(-c6cccc(-c7ccccc7)c6)n5)c4-n4c5ccccc5c5ccccc54)c([2H])c([2H])c([2H])c3c2c1[2H]]

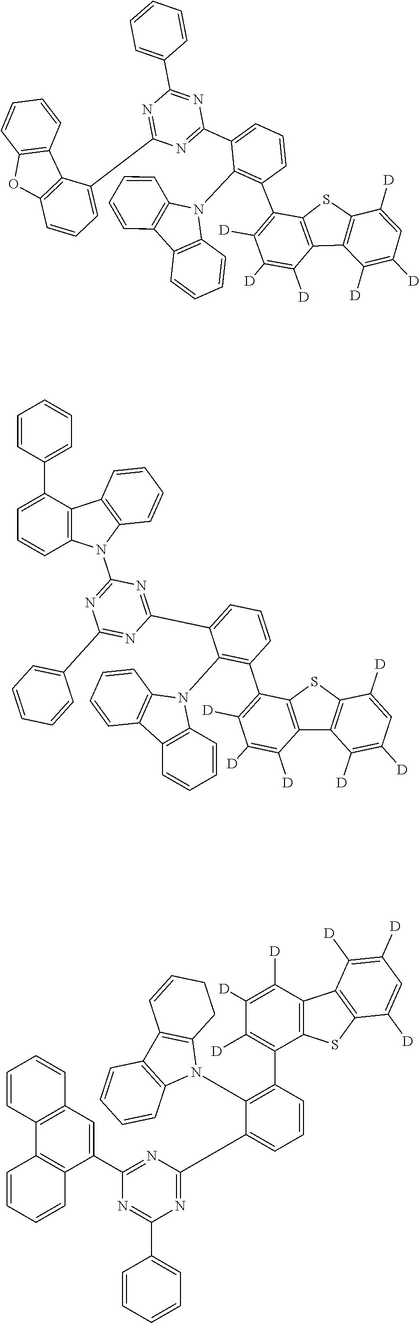 [SMILES: [2H]c1cc([2H])c2sc3c(-c4cccc(-c5nc(-c6ccccc6)nc(-c6cccc7oc8ccccc8c67)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-n6c7ccccc7c7c(-c8ccccc8)cccc76)n5)c4-n4c5ccccc5c5ccccc54)c([2H])c([2H])c([2H])c3c2c1[2H] ; [2H]c1cc([2H])c2sc3c(-c4cccc(-c5nc(-c6ccccc6)nc(-c6cc7ccccc7c7ccccc67)n5)c4-n4c5c(c6ccccc64)C=CCC5)c([2H])c([2H])c([2H])c3c2c1[2H]]

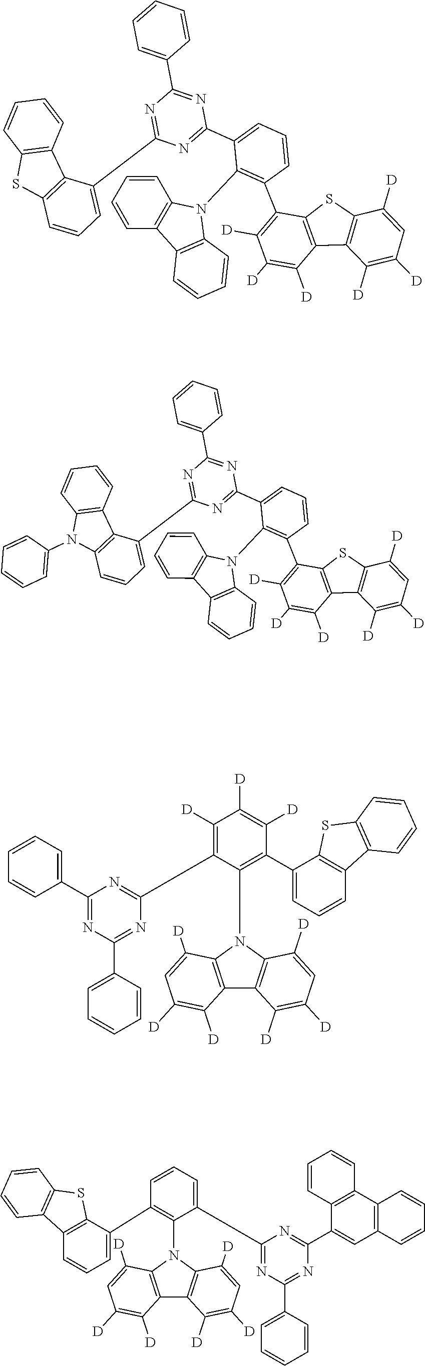

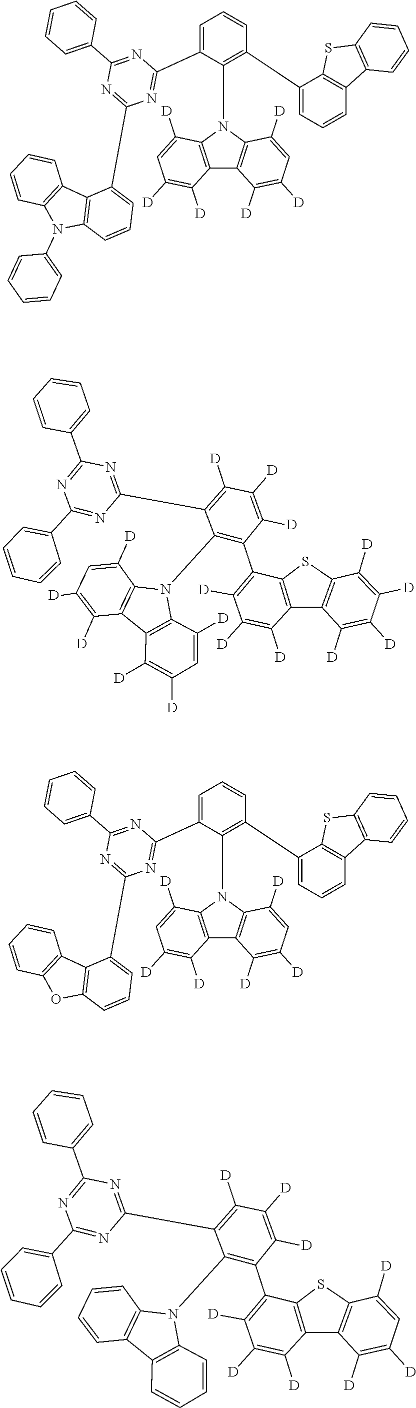

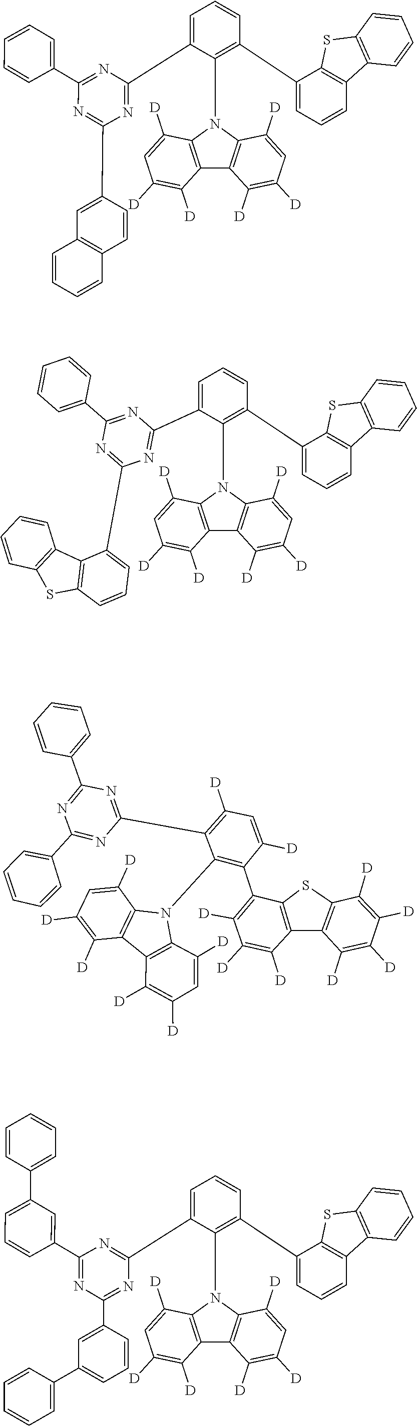

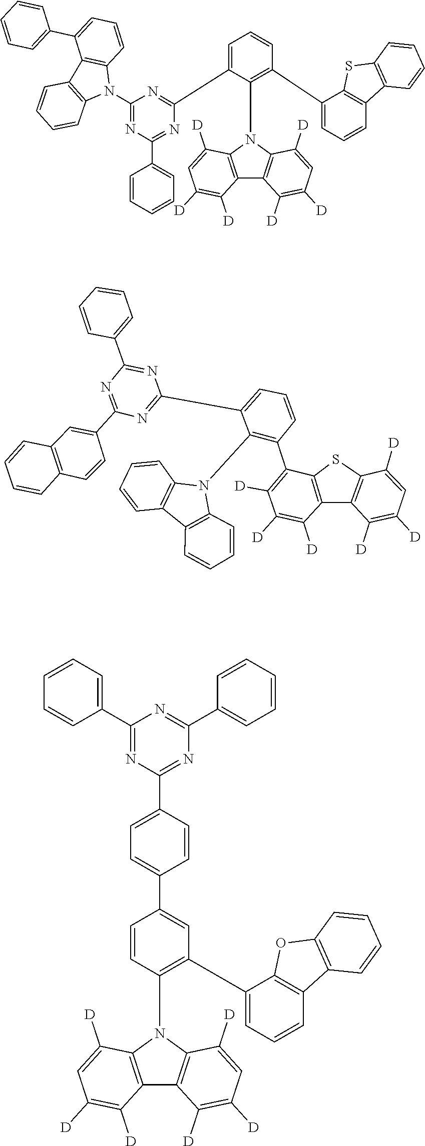 [SMILES: [2H]c1cc([2H])c2c(c1[2H])c1c([2H])c([2H])cc([2H])c1n2-c1c(-c2nc(-c3ccccc3)nc(-n3c4ccccc4c4c(-c5ccccc5)cccc43)n2)cccc1-c1cccc2c1sc1ccccc12 ; [2H]c1cc([2H])c2sc3c(-c4cccc(-c5nc(-c6ccccc6)nc(-c6ccc7ccccc7c6)n5)c4-n4c5ccccc5c5ccccc54)c([2H])c([2H])c([2H])c3c2c1[2H] ; [2H]c1cc([2H])c2c(c1[2H])c1c([2H])c([2H])cc([2H])c1n2-c1ccc(-c2ccc(-c3nc(-c4ccccc4)nc(-c4ccccc4)n3)cc2)cc1-c1cccc2c1oc1ccccc12]

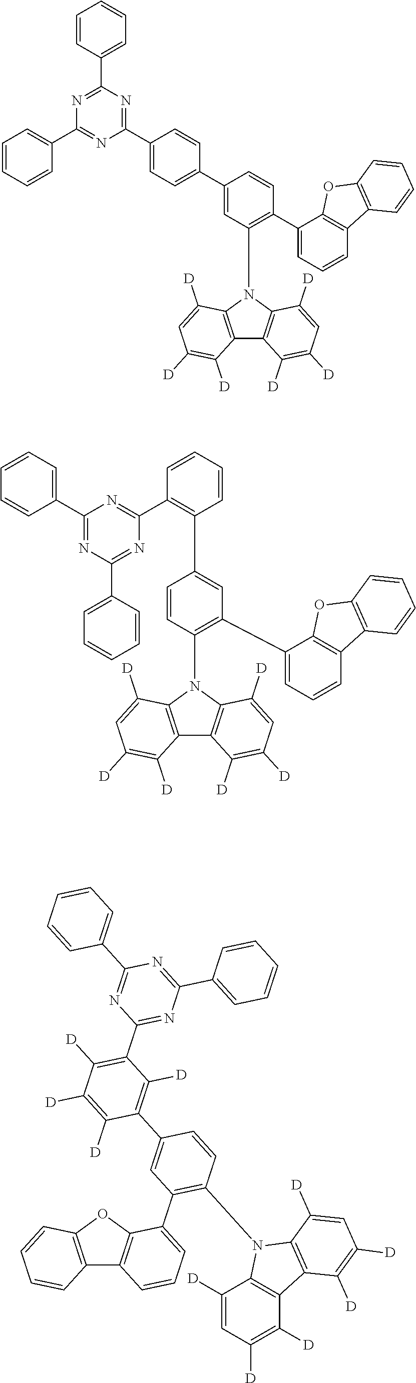 [SMILES: [2H]c1cc([2H])c2c(c1[2H])c1c([2H])c([2H])cc([2H])c1n2-c1cc(-c2ccc(-c3nc(-c4ccccc4)nc(-c4ccccc4)n3)cc2)ccc1-c1cccc2c1oc1ccccc12 ; [2H]c1cc([2H])c2c(c1[2H])c1c([2H])c([2H])cc([2H])c1n2-c1ccc(-c2ccccc2-c2nc(-c3ccccc3)nc(-c3ccccc3)n2)cc1-c1cccc2c1oc1ccccc12 ; [2H]c1cc([2H])c2c(c1[2H])c1c([2H])c([2H])cc([2H])c1n2-c1ccc(-c2c([2H])c([2H])c([2H])c(-c3nc(-c4ccccc4)nc(-c4ccccc4)n3)c2[2H])cc1-c1cccc2c1oc1ccccc12]

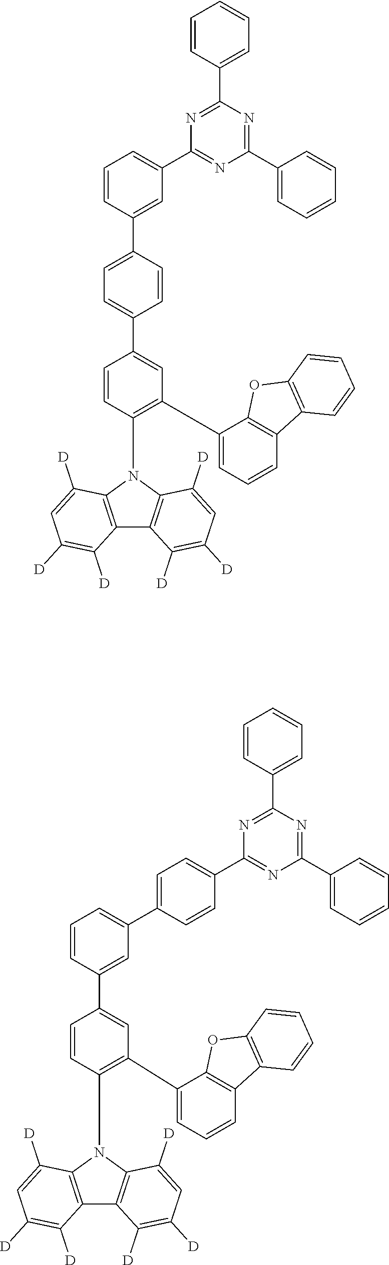 [SMILES: [2H]c1cc([2H])c2c(c1[2H])c1c([2H])c([2H])cc([2H])c1n2-c1ccc(-c2ccc(-c3cccc(-c4nc(-c5ccccc5)nc(-c5ccccc5)n4)c3)cc2)cc1-c1cccc2c1oc1ccccc12 ; [2H]c1cc([2H])c2c(c1[2H])c1c([2H])c([2H])cc([2H])c1n2-c1ccc(-c2cccc(-c3ccc(-c4nc(-c5ccccc5)nc(-c5ccccc5)n4)cc3)c2)cc1-c1cccc2c1oc1ccccc12]

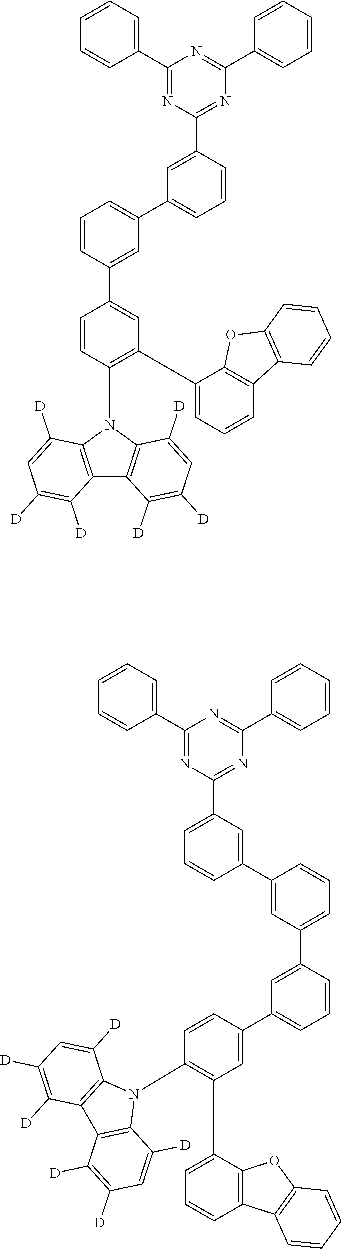 [SMILES: [2H]c1cc([2H])c2c(c1[2H])c1c([2H])c([2H])cc([2H])c1n2-c1ccc(-c2cccc(-c3cccc(-c4nc(-c5ccccc5)nc(-c5ccccc5)n4)c3)c2)cc1-c1cccc2c1oc1ccccc12 ; [2H]c1cc([2H])c2c(c1[2H])c1c([2H])c([2H])cc([2H])c1n2-c1ccc(-c2cccc(-c3cccc(-c4cccc(-c5nc(-c6ccccc6)nc(-c6ccccc6)n5)c4)c3)c2)cc1-c1cccc2c1oc1ccccc12]

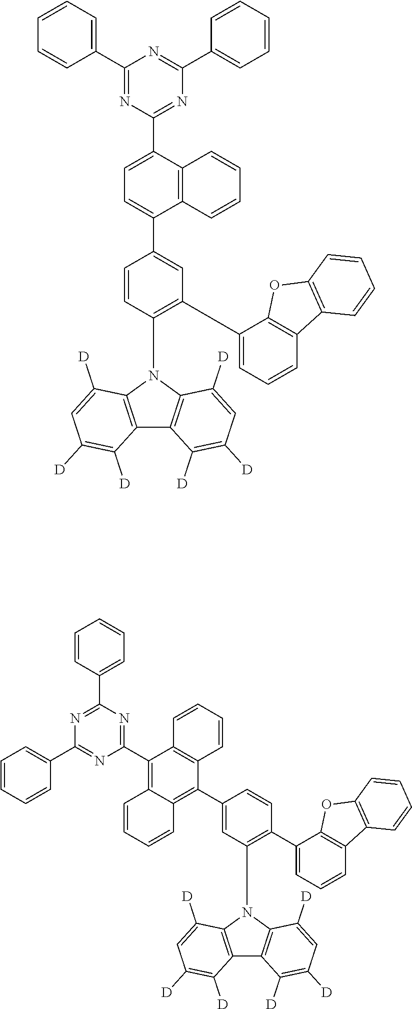 [SMILES: [2H]c1cc([2H])c2c(c1[2H])c1c([2H])c([2H])cc([2H])c1n2-c1ccc(-c2ccc(-c3nc(-c4ccccc4)nc(-c4ccccc4)n3)c3ccccc23)cc1-c1cccc2c1oc1ccccc12 ; [2H]c1cc([2H])c2c(c1[2H])c1c([2H])c([2H])cc([2H])c1n2-c1cc(-c2c3ccccc3c(-c3nc(-c4ccccc4)nc(-c4ccccc4)n3)c3ccccc23)ccc1-c1cccc2c1oc1ccccc12]

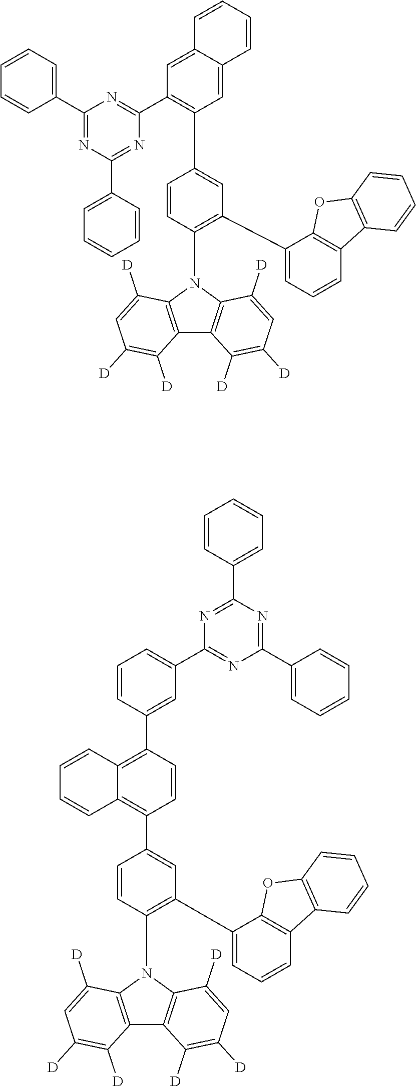 [SMILES: [2H]c1cc([2H])c2c(c1[2H])c1c([2H])c([2H])cc([2H])c1n2-c1ccc(-c2cc3ccccc3cc2-c2nc(-c3ccccc3)nc(-c3ccccc3)n2)cc1-c1cccc2c1oc1ccccc12 ; [2H]c1cc([2H])c2c(c1[2H])c1c([2H])c([2H])cc([2H])c1n2-c1ccc(-c2ccc(-c3cccc(-c4nc(-c5ccccc5)nc(-c5ccccc5)n4)c3)c3ccccc23)cc1-c1cccc2c1oc1ccccc12]

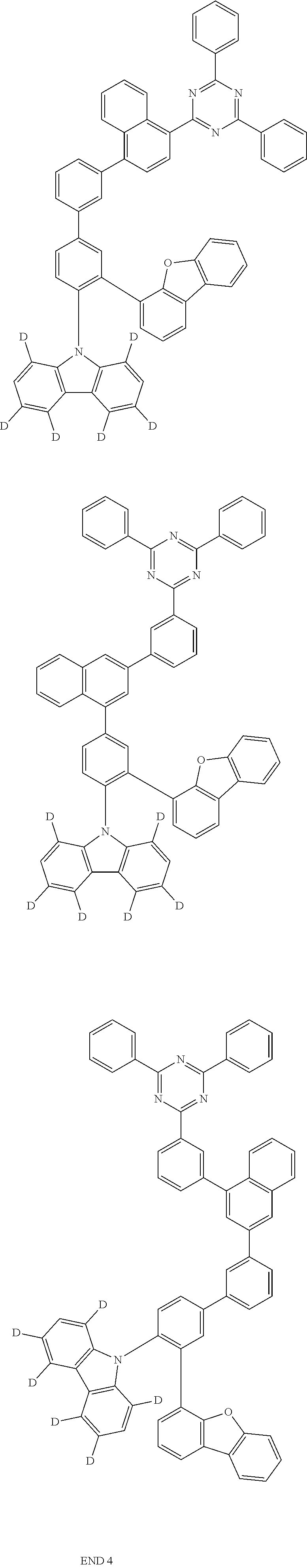 [SMILES: [2H]c1cc([2H])c2c(c1[2H])c1c([2H])c([2H])cc([2H])c1n2-c1ccc(-c2cccc(-c3ccc(-c4nc(-c5ccccc5)nc(-c5ccccc5)n4)c4ccccc34)c2)cc1-c1cccc2c1oc1ccccc12 ; [2H]c1cc([2H])c2c(c1[2H])c1c([2H])c([2H])cc([2H])c1n2-c1ccc(-c2cc(-c3cccc(-c4nc(-c5ccccc5)nc(-c5ccccc5)n4)c3)cc3ccccc23)cc1-c1cccc2c1oc1ccccc12 ; [2H]c1cc([2H])c2c(c1[2H])c1c([2H])c([2H])cc([2H])c1n2-c1ccc(-c2cccc(-c3cc(-c4cccc(-c5nc(-c6ccccc6)nc(-c6ccccc6)n5)c4)c4ccccc4c3)c2)cc1-c1cccc2c1oc1ccccc12]

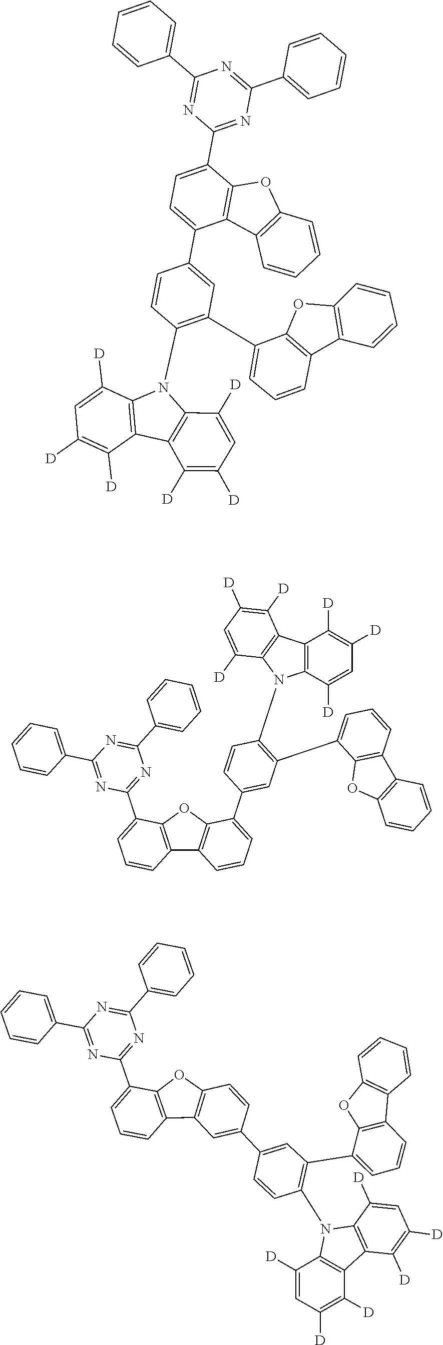 [SMILES: [2H]c1cc([2H])c2c(c1[2H])c1c([2H])c([2H])cc([2H])c1n2-c1ccc(-c2ccc(-c3nc(-c4ccccc4)nc(-c4ccccc4)n3)c3oc4ccccc4c23)cc1-c1cccc2c1oc1ccccc12 ; [2H]c1cc([2H])c2c(c1[2H])c1c([2H])c([2H])cc([2H])c1n2-c1ccc(-c2cccc3c2oc2c(-c4nc(-c5ccccc5)nc(-c5ccccc5)n4)cccc23)cc1-c1cccc2c1oc1ccccc12 ; [2H]c1cc([2H])c2c(c1[2H])c1c([2H])c([2H])cc([2H])c1n2-c1ccc(-c2ccc3oc4c(-c5nc(-c6ccccc6)nc(-c6ccccc6)n5)cccc4c3c2)cc1-c1cccc2c1oc1ccccc12]

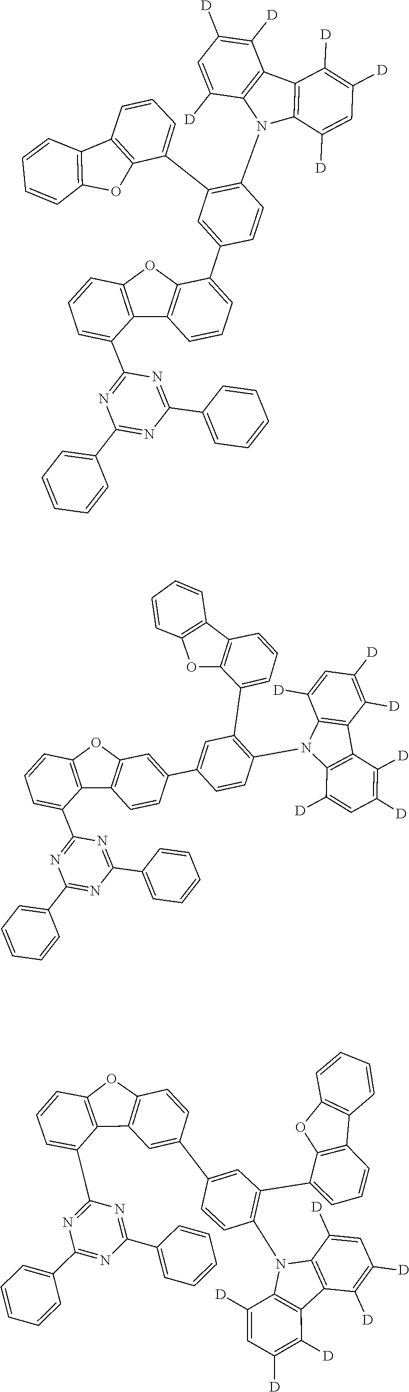 [SMILES: [2H]c1cc([2H])c2c(c1[2H])c1c([2H])c([2H])cc([2H])c1n2-c1ccc(-c2cccc3c2oc2cccc(-c4nc(-c5ccccc5)nc(-c5ccccc5)n4)c23)cc1-c1cccc2c1oc1ccccc12 ; [2H]c1cc([2H])c2c(c1[2H])c1c([2H])c([2H])cc([2H])c1n2-c1ccc(-c2ccc3c(c2)oc2cccc(-c4nc(-c5ccccc5)nc(-c5ccccc5)n4)c23)cc1-c1cccc2c1oc1ccccc12 ; [2H]c1cc([2H])c2c(c1[2H])c1c([2H])c([2H])cc([2H])c1n2-c1ccc(-c2ccc3oc4cccc(-c5nc(-c6ccccc6)nc(-c6ccccc6)n5)c4c3c2)cc1-c1cccc2c1oc1ccccc12]

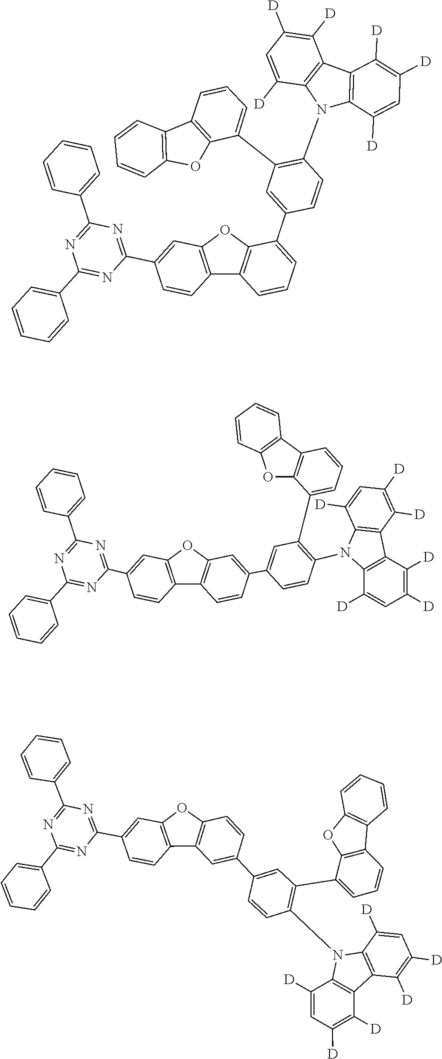 [SMILES: [2H]c1cc([2H])c2c(c1[2H])c1c([2H])c([2H])cc([2H])c1n2-c1ccc(-c2cccc3c2oc2cc(-c4nc(-c5ccccc5)nc(-c5ccccc5)n4)ccc23)cc1-c1cccc2c1oc1ccccc12 ; [2H]c1cc([2H])c2c(c1[2H])c1c([2H])c([2H])cc([2H])c1n2-c1ccc(-c2ccc3c(c2)oc2cc(-c4nc(-c5ccccc5)nc(-c5ccccc5)n4)ccc23)cc1-c1cccc2c1oc1ccccc12 ; [2H]c1cc([2H])c2c(c1[2H])c1c([2H])c([2H])cc([2H])c1n2-c1ccc(-c2ccc3oc4cc(-c5nc(-c6ccccc6)nc(-c6ccccc6)n5)ccc4c3c2)cc1-c1cccc2c1oc1ccccc12]

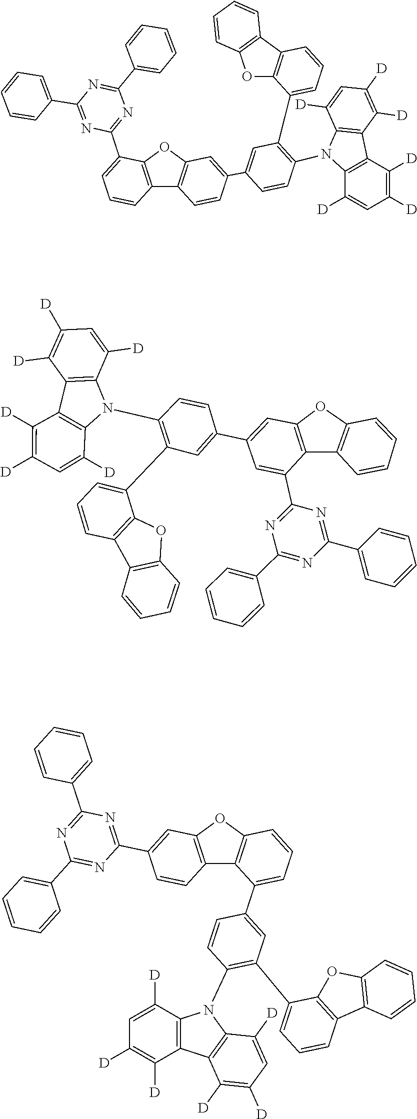 [SMILES: [2H]c1cc([2H])c2c(c1[2H])c1c([2H])c([2H])cc([2H])c1n2-c1ccc(-c2ccc3c(c2)oc2c(-c4nc(-c5ccccc5)nc(-c5ccccc5)n4)cccc23)cc1-c1cccc2c1oc1ccccc12 ; [2H]c1cc([2H])c2c(c1[2H])c1c([2H])c([2H])cc([2H])c1n2-c1ccc(-c2cc(-c3nc(-c4ccccc4)nc(-c4ccccc4)n3)c3c(c2)oc2ccccc23)cc1-c1cccc2c1oc1ccccc12 ; [2H]c1cc([2H])c2c(c1[2H])c1c([2H])c([2H])cc([2H])c1n2-c1ccc(-c2cccc3oc4cc(-c5nc(-c6ccccc6)nc(-c6ccccc6)n5)ccc4c23)cc1-c1cccc2c1oc1ccccc12]

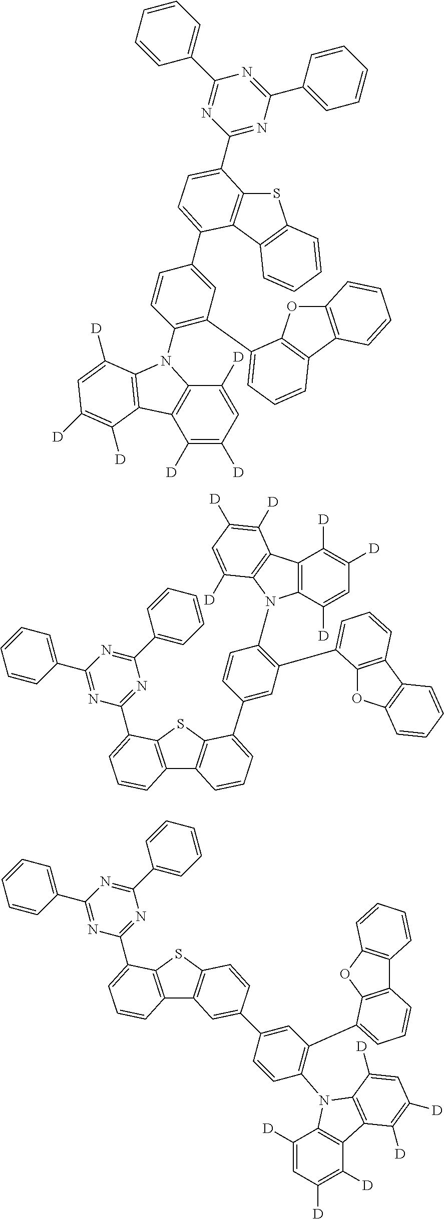

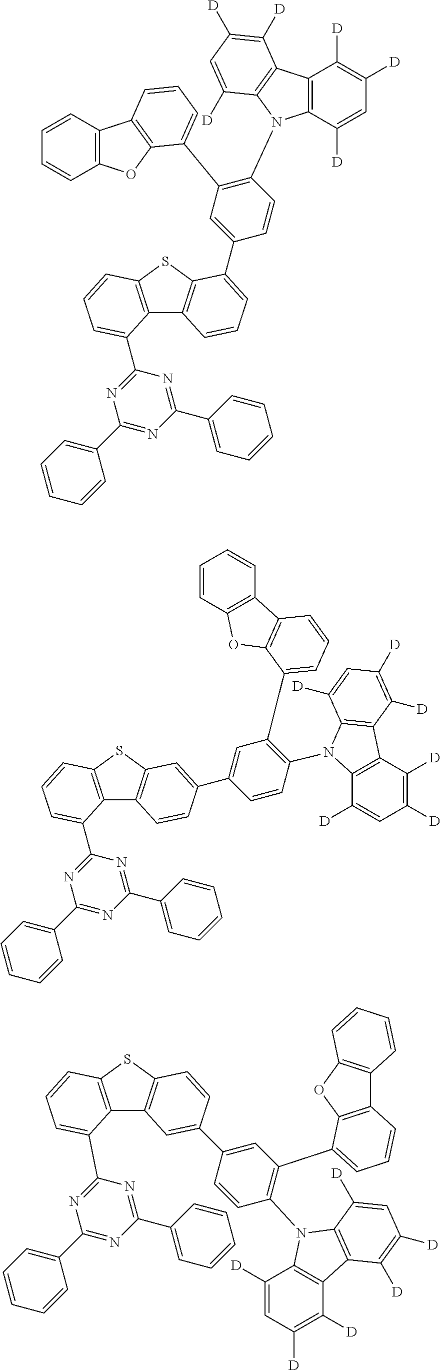 [SMILES: [2H]c1cc([2H])c2c(c1[2H])c1c([2H])c([2H])cc([2H])c1n2-c1ccc(-c2cccc3c2sc2cccc(-c4nc(-c5ccccc5)nc(-c5ccccc5)n4)c23)cc1-c1cccc2c1oc1ccccc12 ; [2H]c1cc([2H])c2c(c1[2H])c1c([2H])c([2H])cc([2H])c1n2-c1ccc(-c2ccc3c(c2)sc2cccc(-c4nc(-c5ccccc5)nc(-c5ccccc5)n4)c23)cc1-c1cccc2c1oc1ccccc12 ; [2H]c1cc([2H])c2c(c1[2H])c1c([2H])c([2H])cc([2H])c1n2-c1ccc(-c2ccc3sc4cccc(-c5nc(-c6ccccc6)nc(-c6ccccc6)n5)c4c3c2)cc1-c1cccc2c1oc1ccccc12]

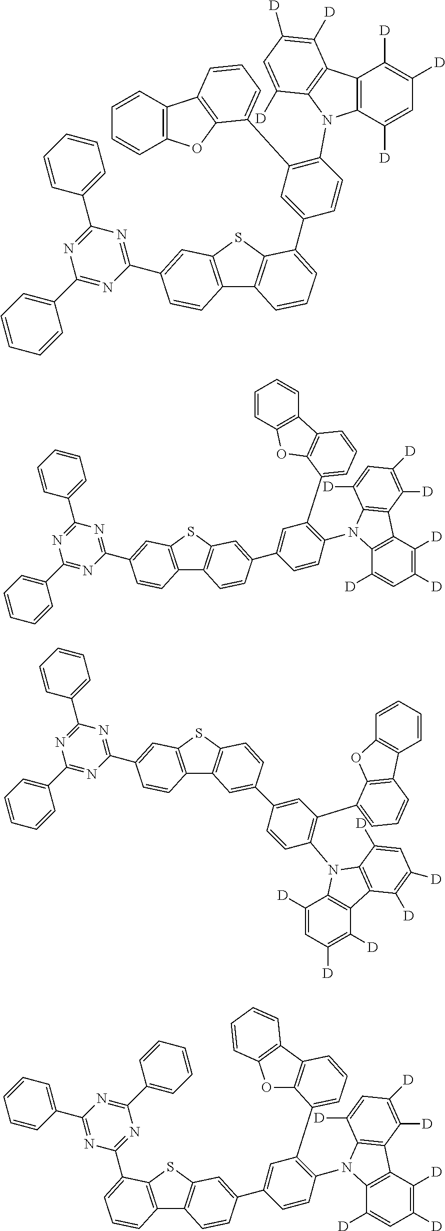

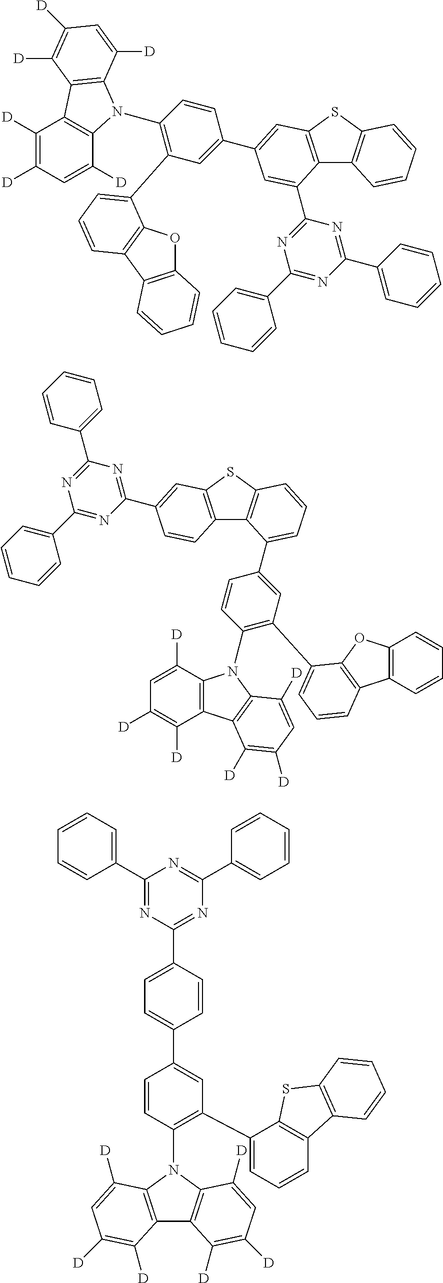

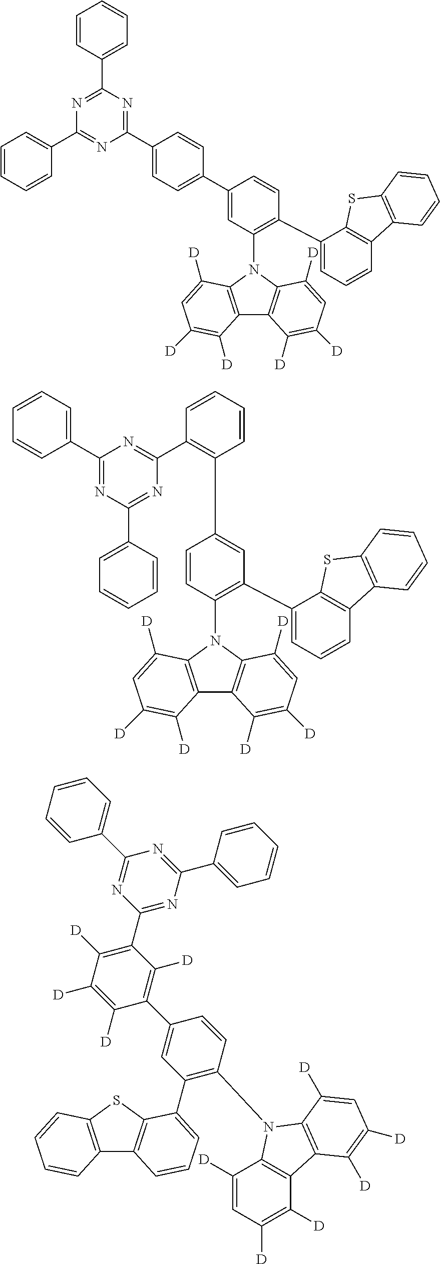

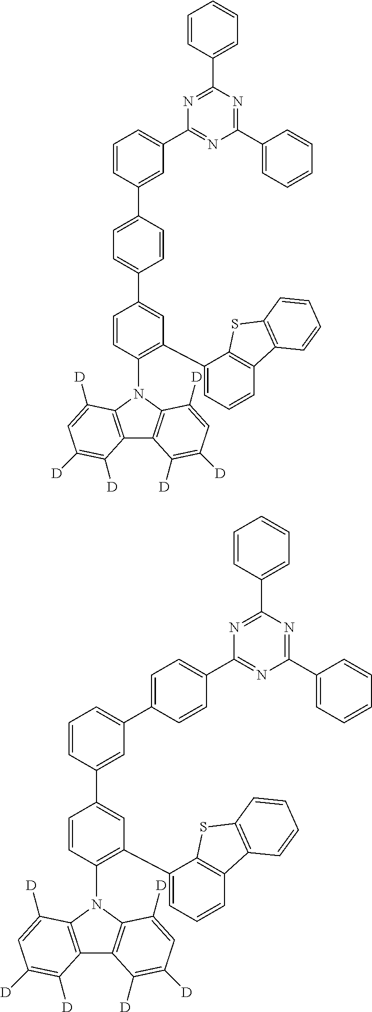 [SMILES: [2H]c1cc([2H])c2c(c1[2H])c1c([2H])c([2H])cc([2H])c1n2-c1ccc(-c2ccc(-c3cccc(-c4nc(-c5ccccc5)nc(-c5ccccc5)n4)c3)cc2)cc1-c1cccc2c1sc1ccccc12 ; [2H]c1cc([2H])c2c(c1[2H])c1c([2H])c([2H])cc([2H])c1n2-c1ccc(-c2cccc(-c3ccc(-c4nc(-c5ccccc5)nc(-c5ccccc5)n4)cc3)c2)cc1-c1cccc2c1sc1ccccc12]

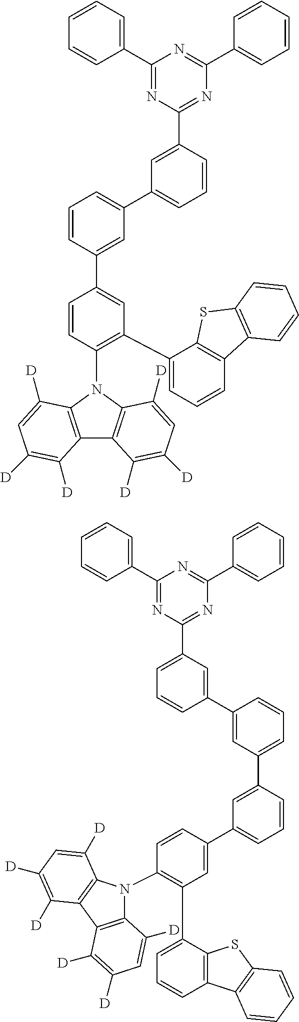

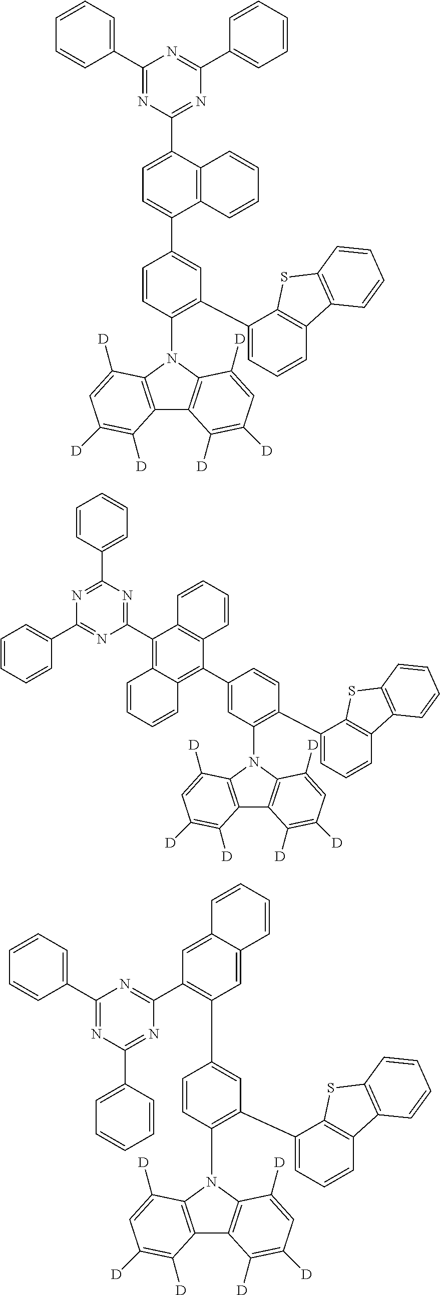

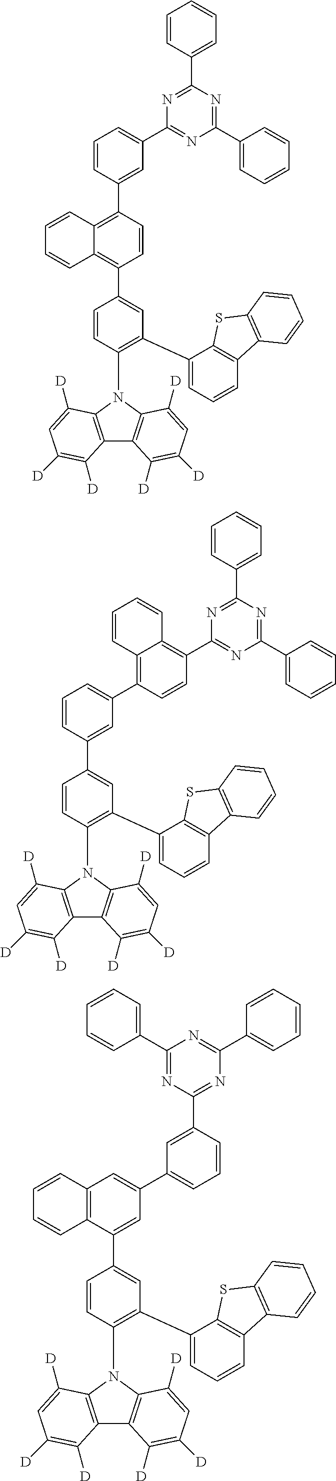

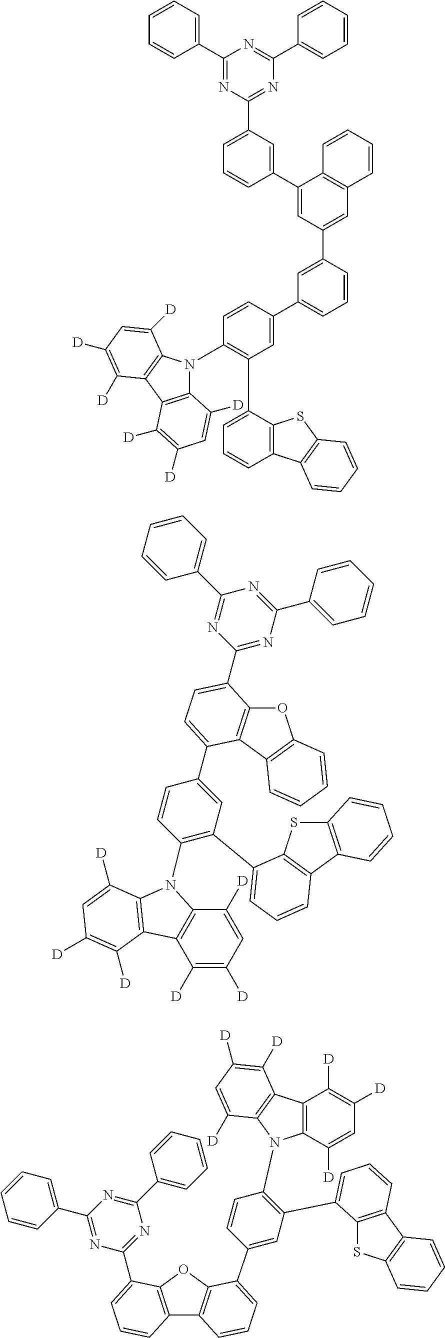

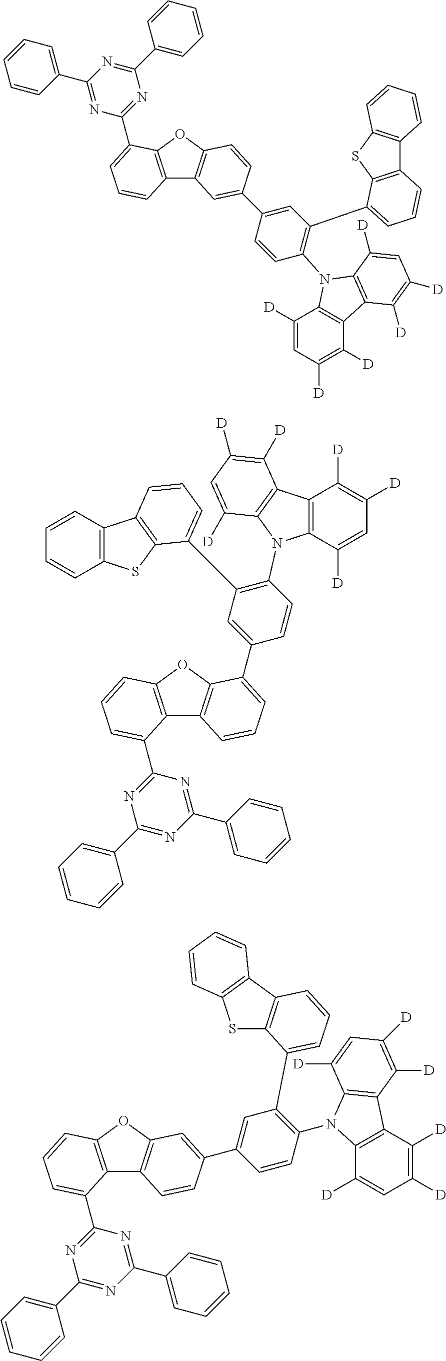 [SMILES: [2H]c1cc([2H])c2c(c1[2H])c1c([2H])c([2H])cc([2H])c1n2-c1ccc(-c2ccc3oc4c(-c5nc(-c6ccccc6)nc(-c6ccccc6)n5)cccc4c3c2)cc1-c1cccc2c1sc1ccccc12 ; [2H]c1cc([2H])c2c(c1[2H])c1c([2H])c([2H])cc([2H])c1n2-c1ccc(-c2cccc3c2oc2cccc(-c4nc(-c5ccccc5)nc(-c5ccccc5)n4)c23)cc1-c1cccc2c1sc1ccccc12 ; [2H]c1cc([2H])c2c(c1[2H])c1c([2H])c([2H])cc([2H])c1n2-c1ccc(-c2ccc3c(c2)oc2cccc(-c4nc(-c5ccccc5)nc(-c5ccccc5)n4)c23)cc1-c1cccc2c1sc1ccccc12]

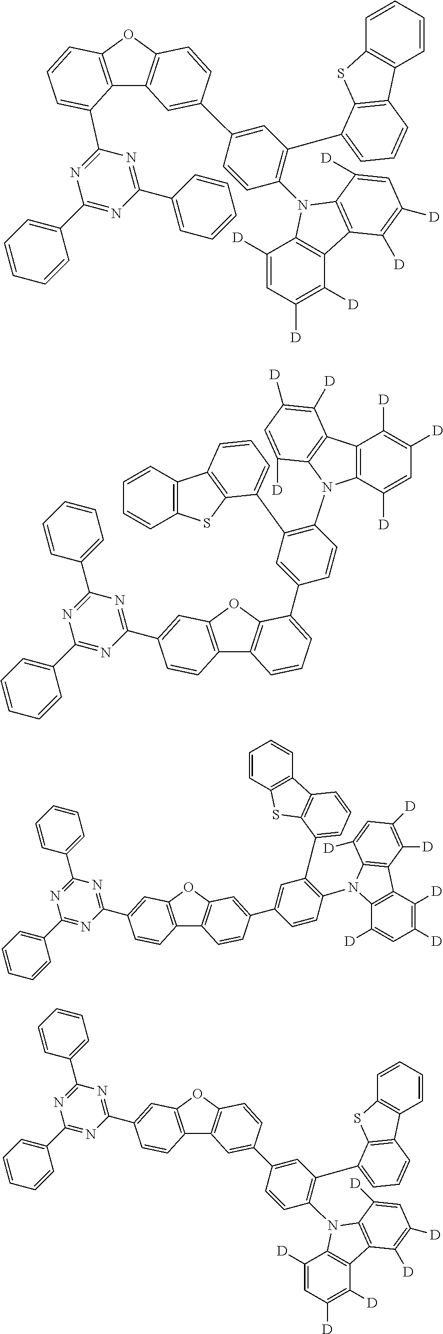

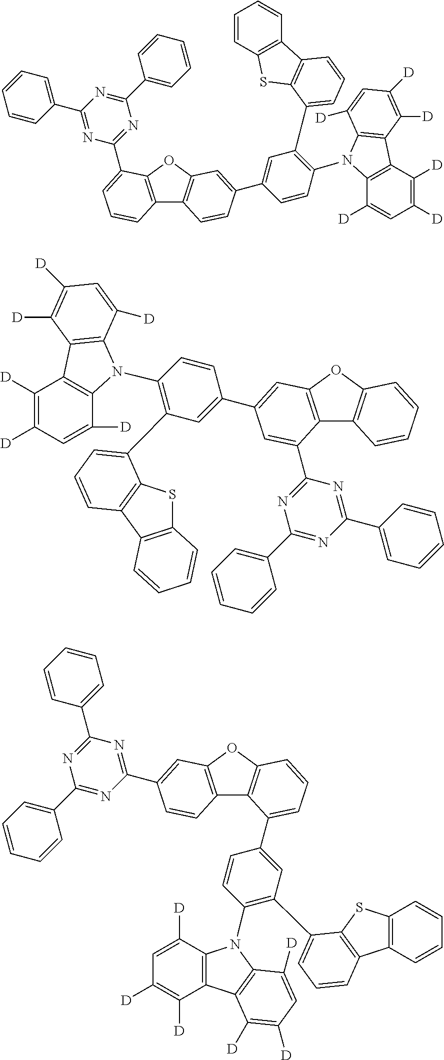

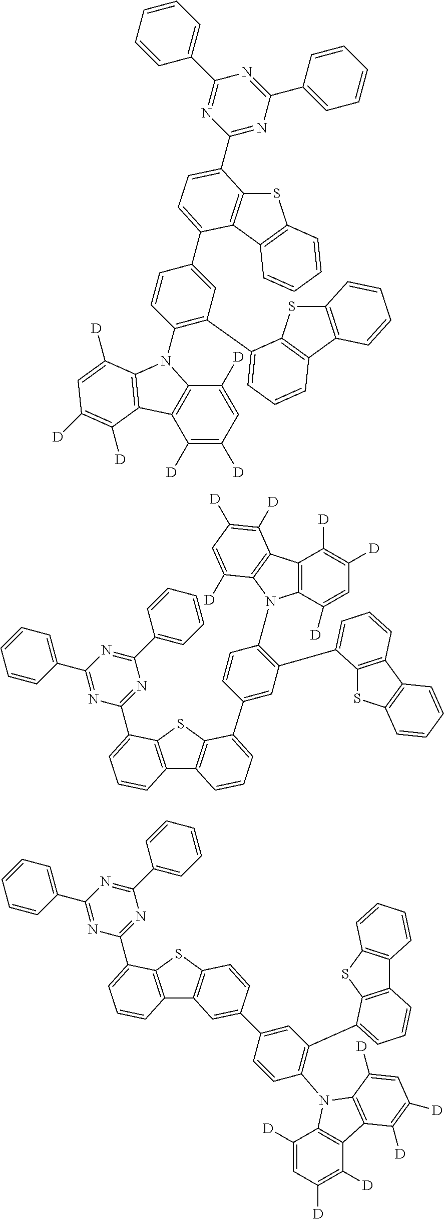

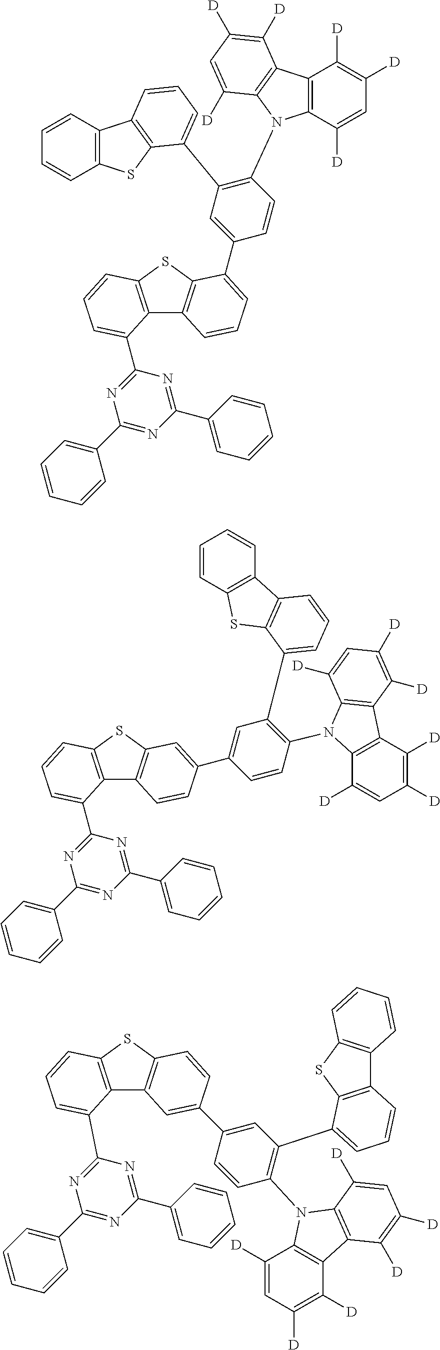 [SMILES: [2H]c1cc([2H])c2c(c1[2H])c1c([2H])c([2H])cc([2H])c1n2-c1ccc(-c2cccc3c2sc2cccc(-c4nc(-c5ccccc5)nc(-c5ccccc5)n4)c23)cc1-c1cccc2c1sc1ccccc12 ; [2H]c1cc([2H])c2c(c1[2H])c1c([2H])c([2H])cc([2H])c1n2-c1ccc(-c2ccc3c(c2)sc2cccc(-c4nc(-c5ccccc5)nc(-c5ccccc5)n4)c23)cc1-c1cccc2c1sc1ccccc12 ; [2H]c1cc([2H])c2c(c1[2H])c1c([2H])c([2H])cc([2H])c1n2-c1ccc(-c2ccc3sc4cccc(-c5nc(-c6ccccc6)nc(-c6ccccc6)n5)c4c3c2)cc1-c1cccc2c1sc1ccccc12]

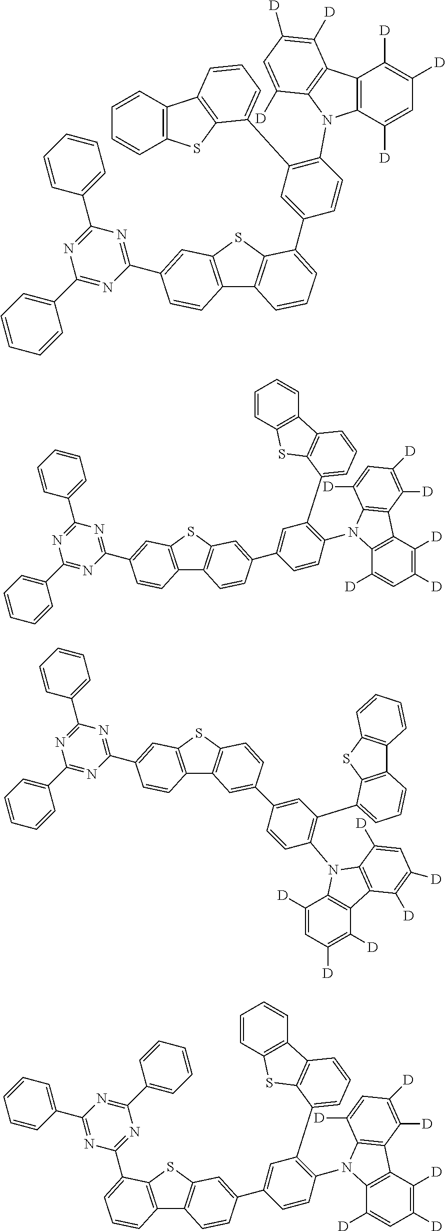

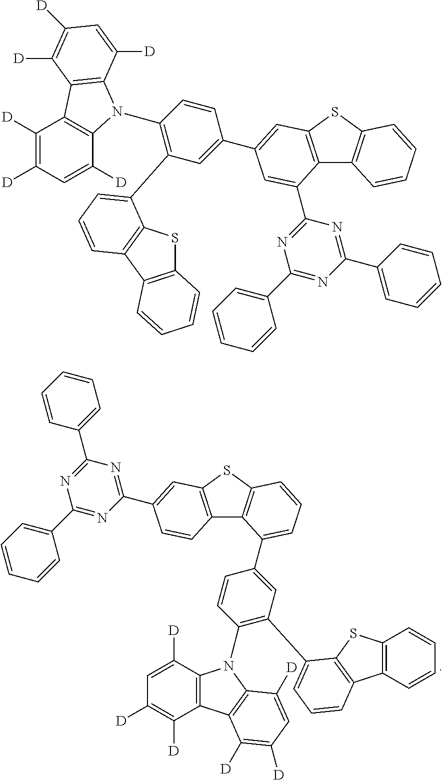 [SMILES: [2H]c1cc([2H])c2c(c1[2H])c1c([2H])c([2H])cc([2H])c1n2-c1ccc(-c2cc(-c3nc(-c4ccccc4)nc(-c4ccccc4)n3)c3c(c2)sc2ccccc23)cc1-c1cccc2c1sc1ccccc12 ; [2H]c1cc([2H])c2c(c1[2H])c1c([2H])c([2H])cc([2H])c1n2-c1ccc(-c2cccc3sc4cc(-c5nc(-c6ccccc6)nc(-c6ccccc6)n5)ccc4c23)cc1-c1cccc2c1sc1ccccc12]

15. An organic light emitting device comprising:
a first electrode;
a second electrode that is opposite to the first electrode; and
one or more organic material layers that are between the first electrode and the second electrode, wherein one or more layers of the one or more organic material layers comprises the compound of claim 1.

16. The organic light emitting device according to claim 15, wherein:
the organic material layer comprising the compound is a light emitting layer.

17. The organic light emitting device according to claim 16, wherein:
the light emitting layer further comprises a compound of Chemical Formula 2: 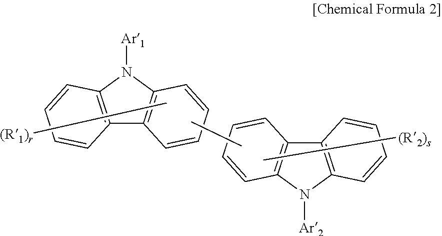
wherein, in Chemical Formula 2;
Ar′₁ and Ar′₂ are each independently a substituted or unsubstituted C₆₋₆₀ aryl or a substituted or unsubstituted C₂₋₆₀ heteroaryl containing one or more heteroatoms of N, O and S;
R′₁ and R′₂ are each independently deuterium, a C₁₋₆₀ alkyl, a C₆₋₆₀ aryl, or a C₂₋₆₀ heteroaryl containing one or more heteroatoms of N, O and S; and
r and s are each independently an integer of 0 to 7.

18. The organic light emitting device according to claim 17, wherein:
the compound of Chemical Formula 2 is any one compound selected from the group consisting of the following compounds: 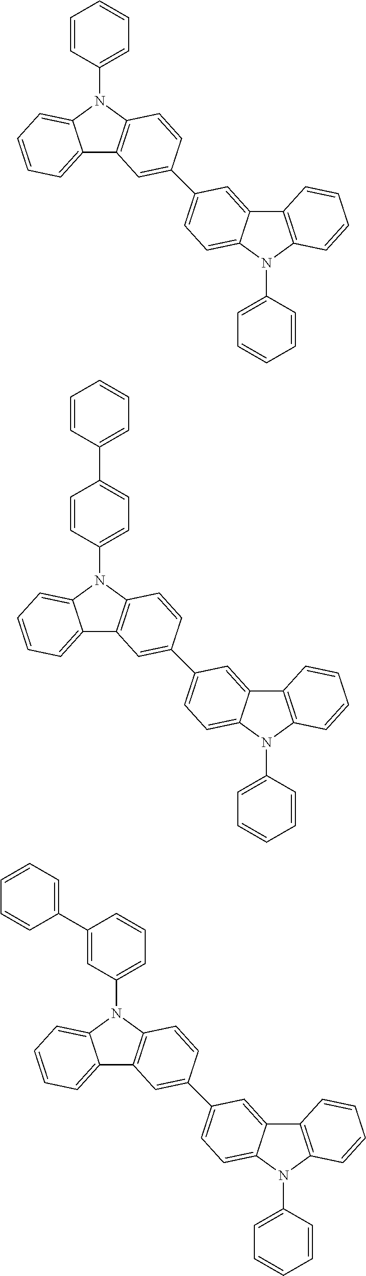 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1] 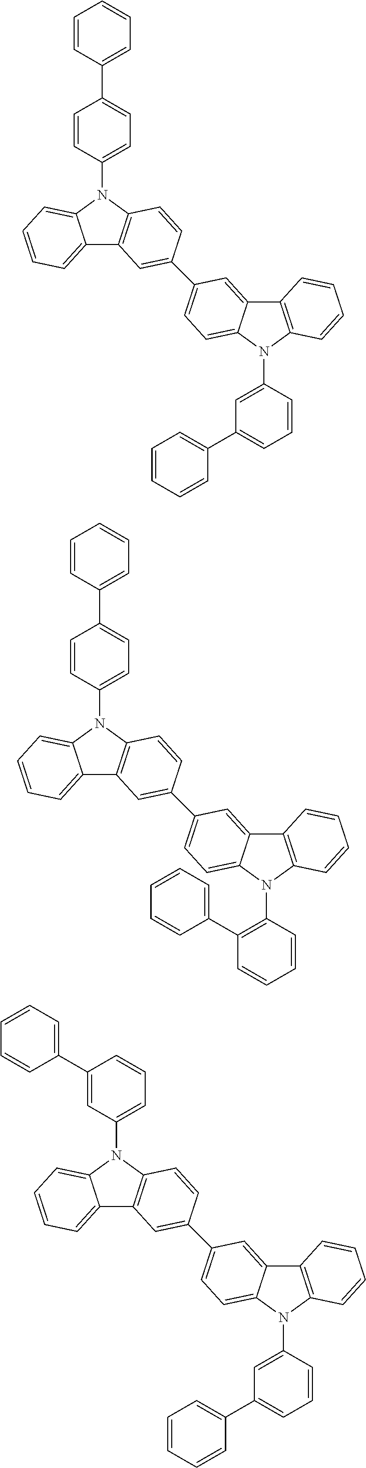 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1] 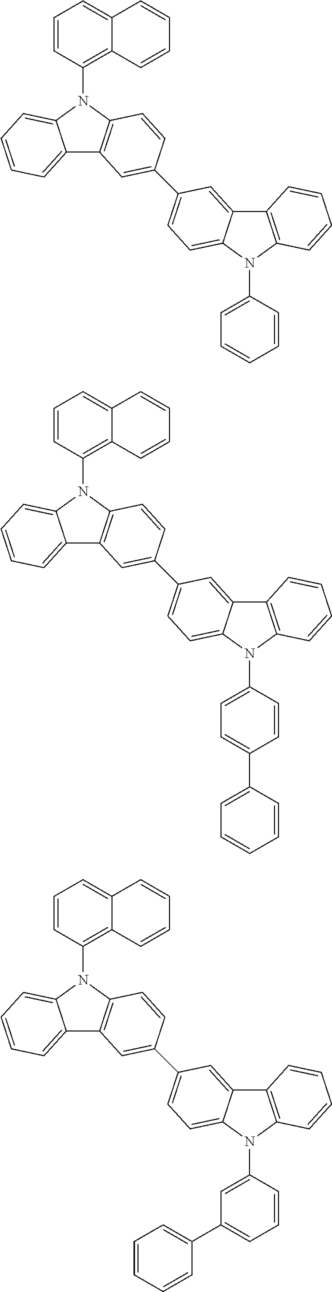 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2cccc(-n3c4ccccc4c4cc(-c5ccc6c(c5)c5ccccc5n6-c5cccc6ccccc56)ccc43)c2)cc1] 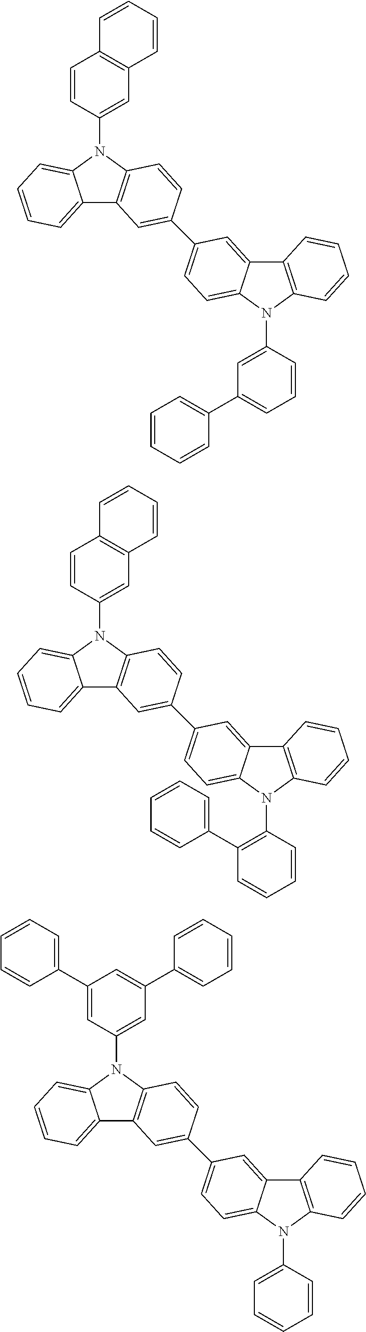 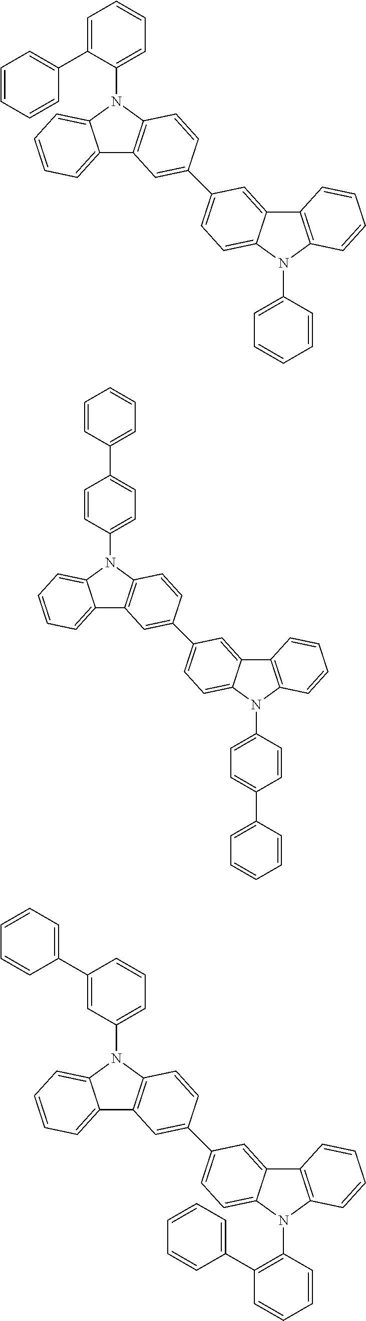 [SMILES: c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1] 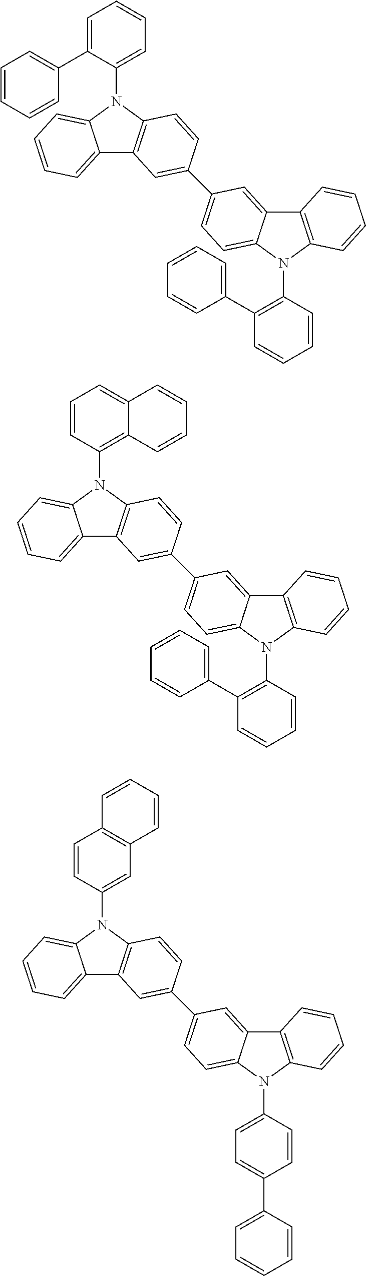 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccccc2-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1] 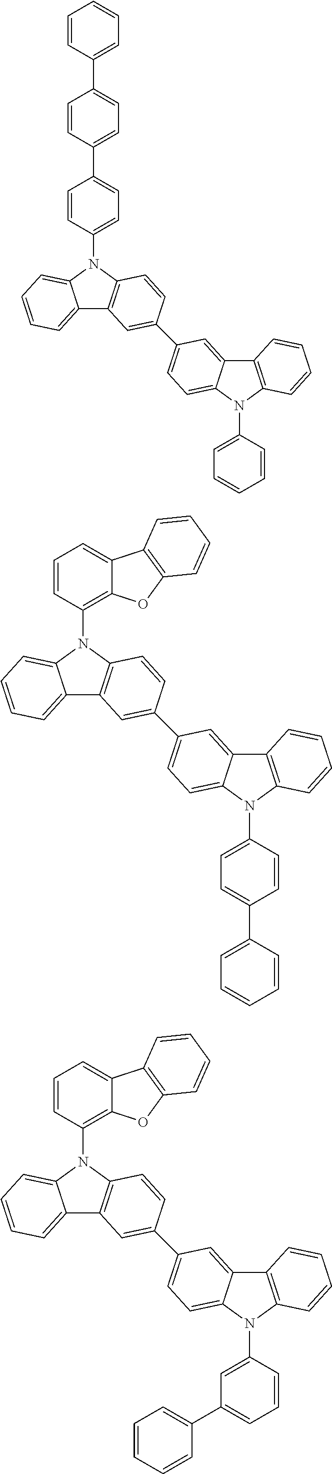 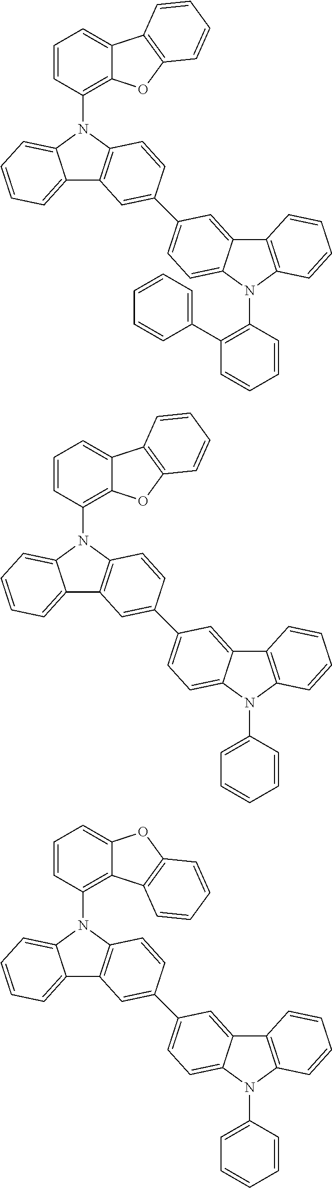 [SMILES: c1ccc(-c2ccccc2-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc5oc6ccccc6c45)ccc32)cc1] 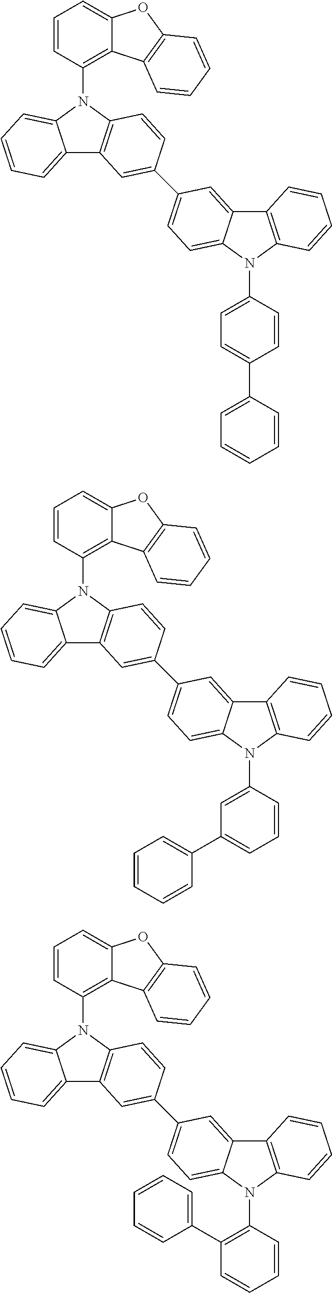 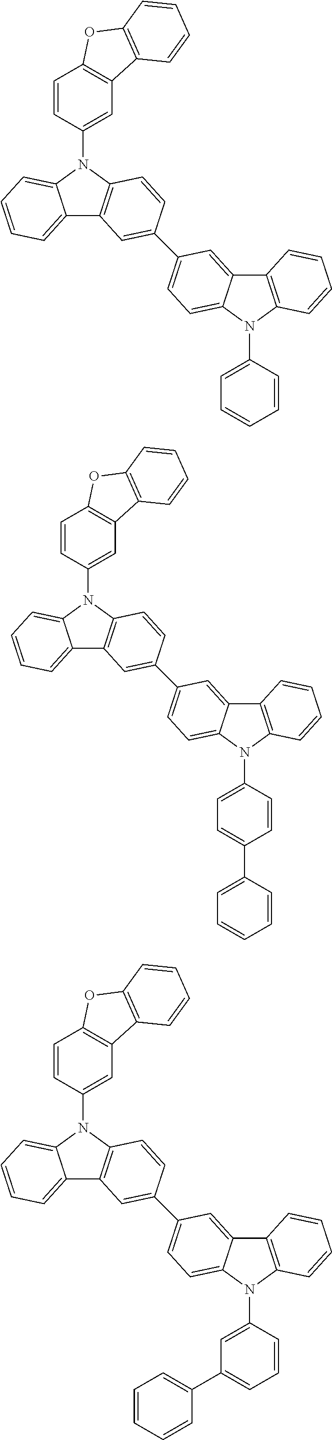 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cccc(-n3c4ccccc4c4cc(-c5ccc6c(c5)c5ccccc5n6-c5ccc6oc7ccccc7c6c5)ccc43)c2)cc1] 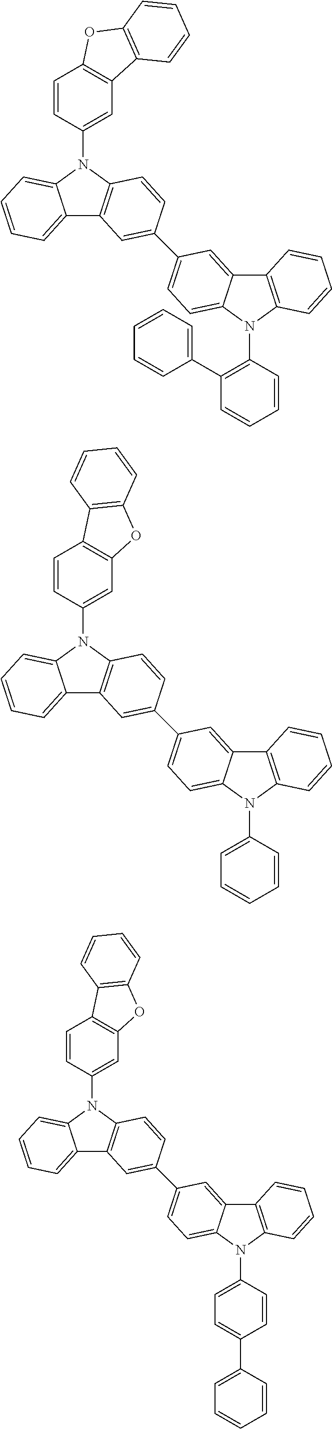 [SMILES: c1ccc(-c2ccccc2-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5c(c4)oc4ccccc45)ccc32)cc1 ; c1ccc(-c2ccc(-n3c4ccccc4c4cc(-c5ccc6c(c5)c5ccccc5n6-c5ccc6c(c5)oc5ccccc56)ccc43)cc2)cc1] 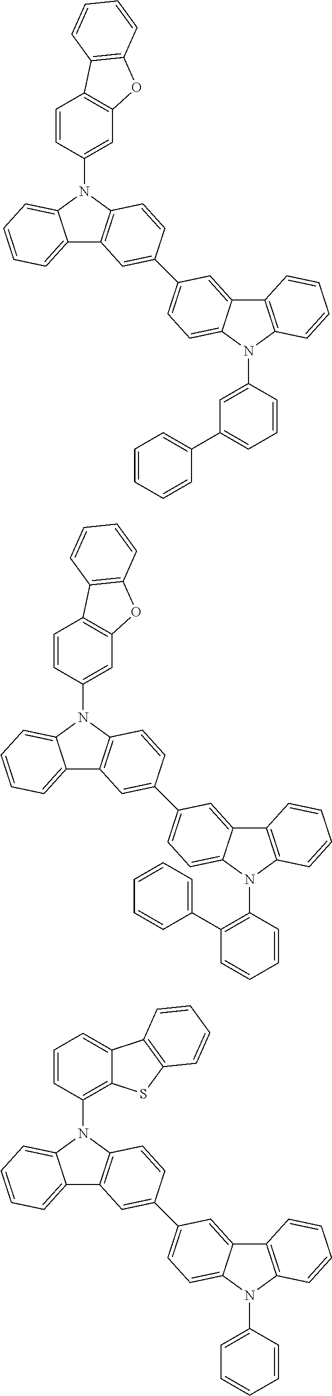 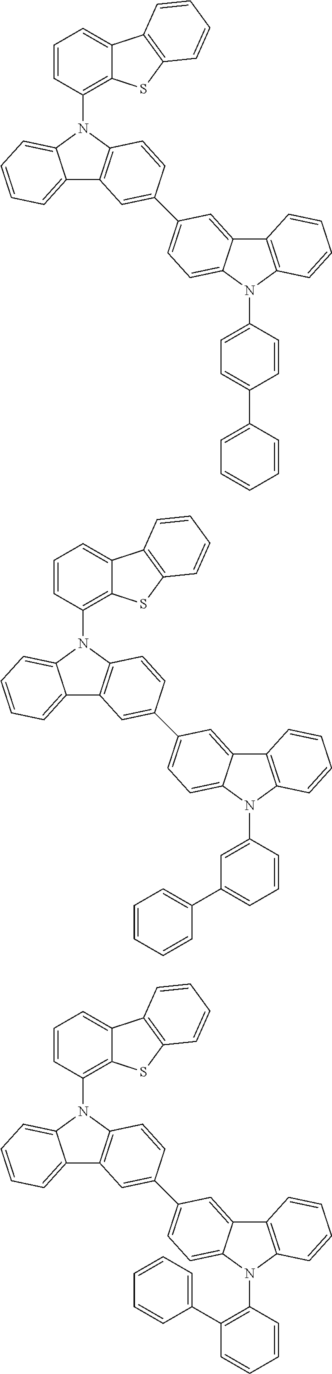 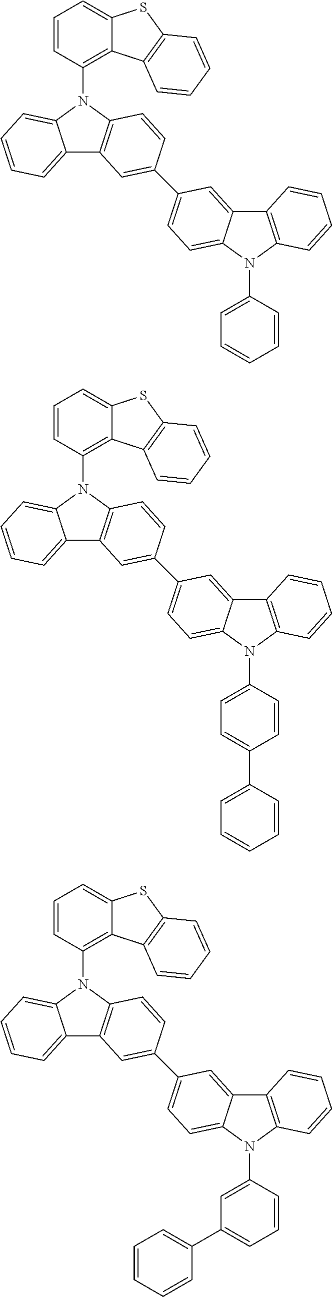 [SMILES: c1ccc(-n2c3ccccc3c3cc(-c4ccc5c(c4)c4ccccc4n5-c4cccc5sc6ccccc6c45)ccc32)cc1 ; c1ccc(-c2ccc(-n3c4ccccc4c4cc(-c5ccc6c(c5)c5ccccc5n6-c5cccc6sc7ccccc7c56)ccc43)cc2)cc1 ; c1ccc(-c2cccc(-n3c4ccccc4c4cc(-c5ccc6c(c5)c5ccccc5n6-c5cccc6sc7ccccc7c56)ccc43)c2)cc1] 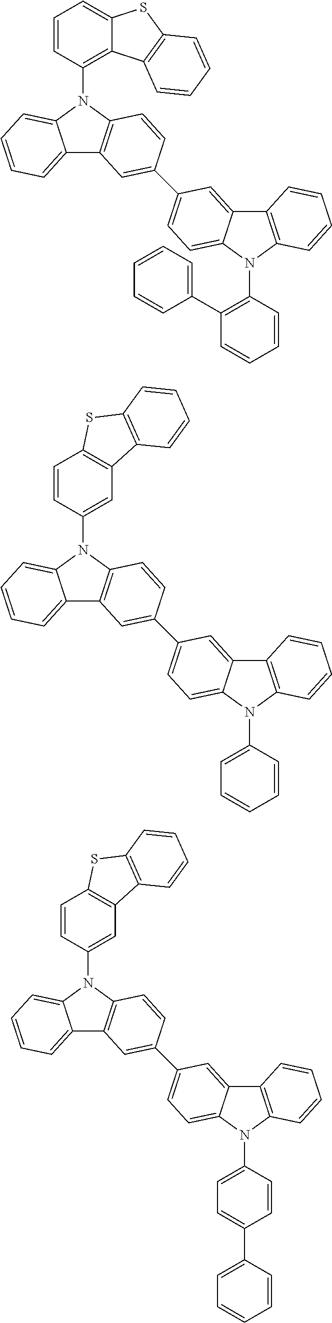 [SMILES: c1ccc(-c2ccccc2-n2c3ccccc3c3cc(-c4ccc5c(c4)c4ccccc4n5-c4cccc5sc6ccccc6c45)ccc32)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1] 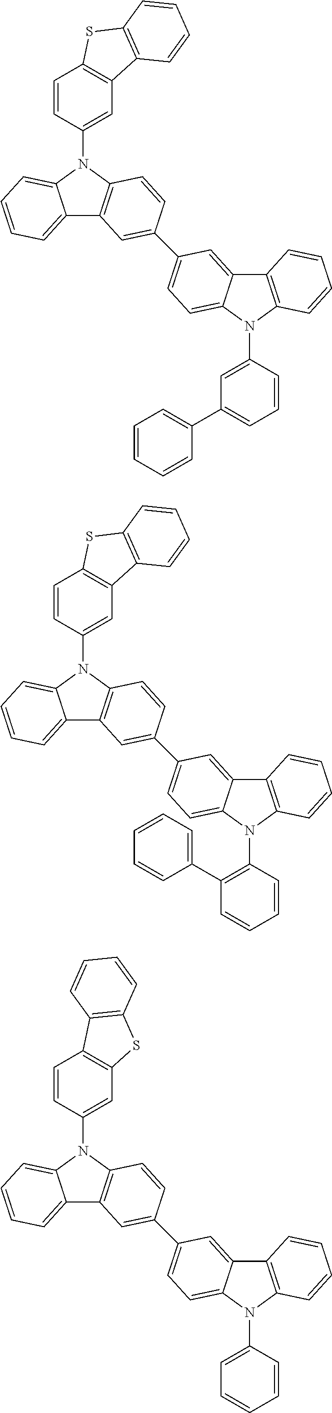 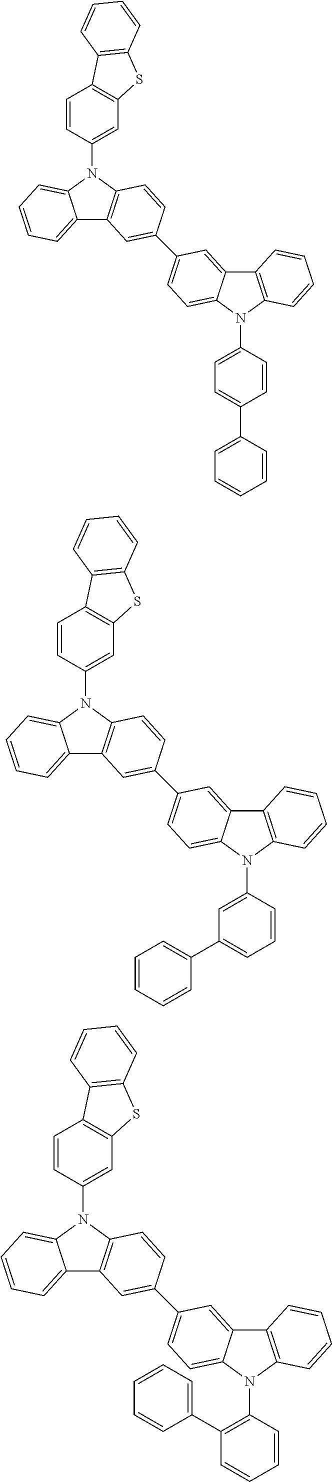 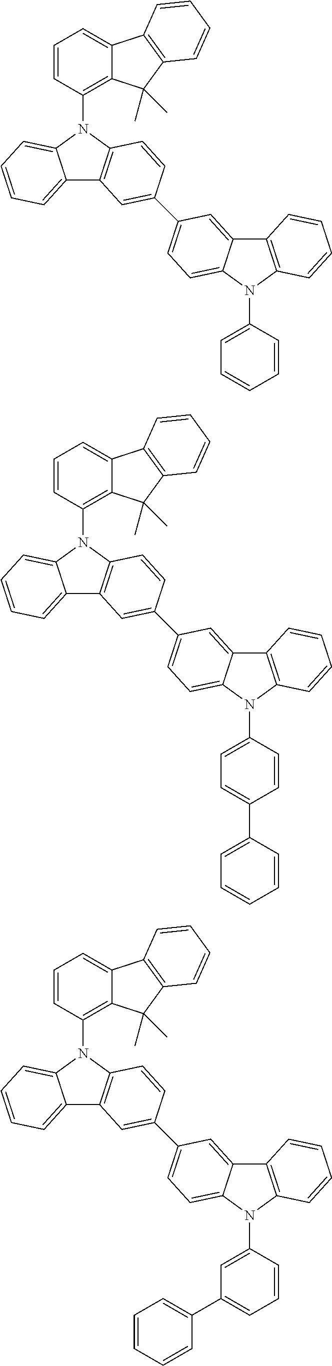 [SMILES: CC1(C)c2ccccc2-c2cccc(-n3c4ccccc4c4cc(-c5ccc6c(c5)c5ccccc5n6-c5ccccc5)ccc43)c21 ; CC1(C)c2ccccc2-c2cccc(-n3c4ccccc4c4cc(-c5ccc6c(c5)c5ccccc5n6-c5ccc(-c6ccccc6)cc5)ccc43)c21 ; CC1(C)c2ccccc2-c2cccc(-n3c4ccccc4c4cc(-c5ccc6c(c5)c5ccccc5n6-c5cccc(-c6ccccc6)c5)ccc43)c21] 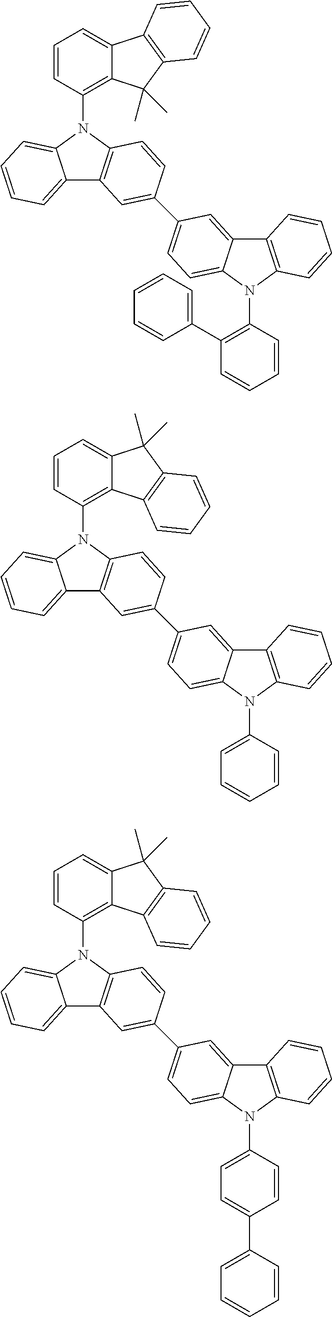 [SMILES: CC1(C)c2ccccc2-c2cccc(-n3c4ccccc4c4cc(-c5ccc6c(c5)c5ccccc5n6-c5ccccc5-c5ccccc5)ccc43)c21 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2c(-n3c4ccccc4c4cc(-c5ccc6c(c5)c5ccccc5n6-c5ccc(-c6ccccc6)cc5)ccc43)cccc21] 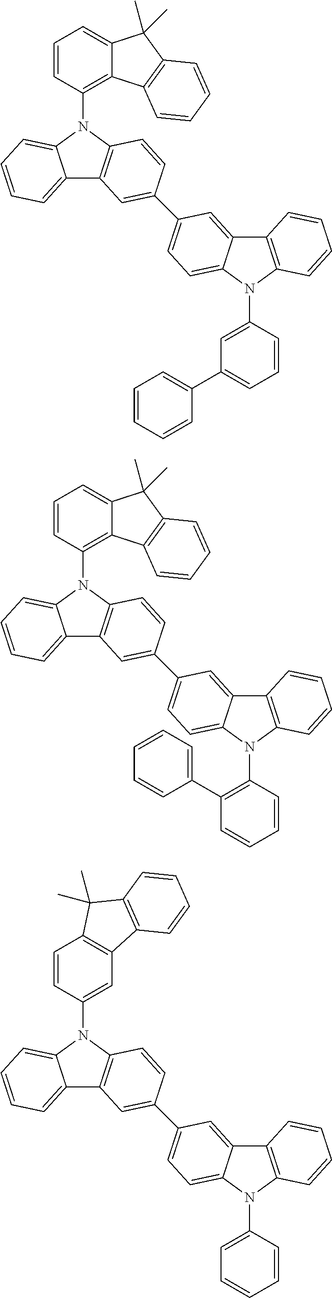 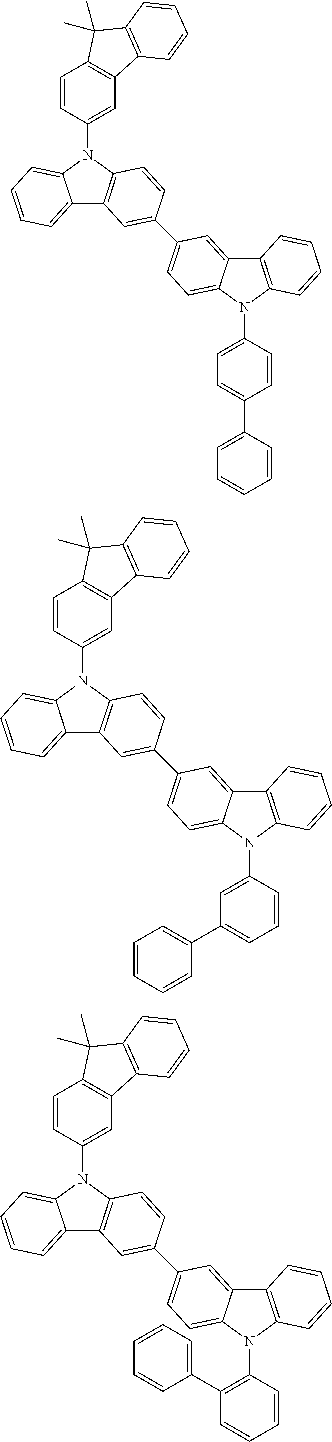 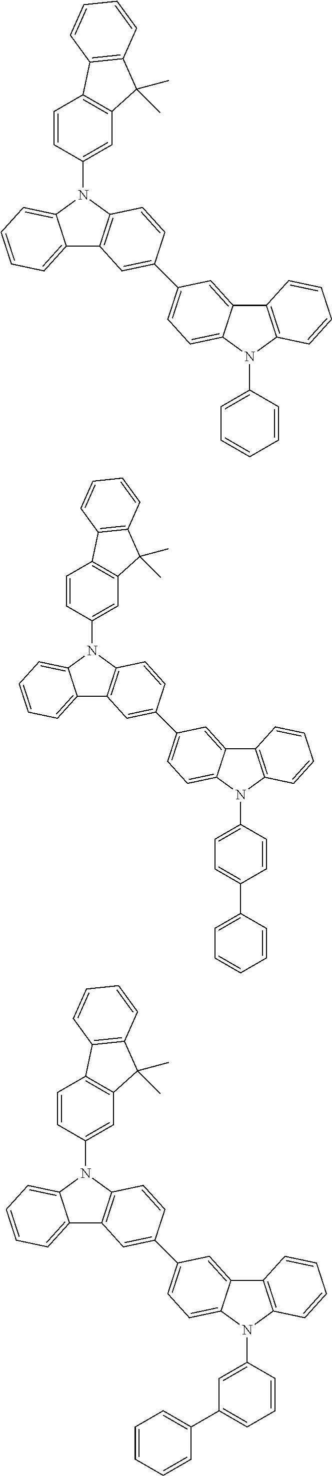 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cccc(-c6ccccc6)c5)ccc43)cc21] 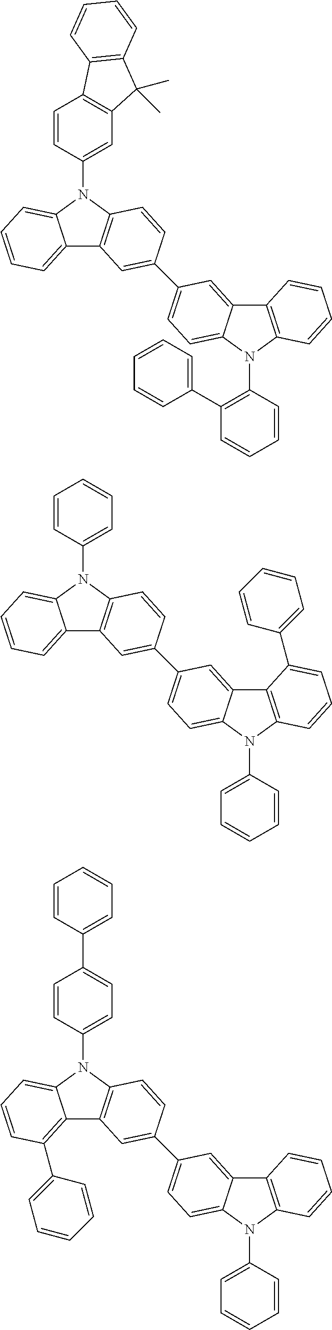 [SMILES: CC1(C)c2ccccc2-c2ccc(-n3c4ccccc4c4cc(-c5ccc6c(c5)c5ccccc5n6-c5ccccc5-c5ccccc5)ccc43)cc21 ; c1ccc(-c2cccc3c2c2cc(-c4ccc5c(c4)c4ccccc4n5-c4ccccc4)ccc2n3-c2ccccc2)cc1 ; c1ccc(-c2ccc(-n3c4ccc(-c5ccc6c(c5)c5ccccc5n6-c5ccccc5)cc4c4c(-c5ccccc5)cccc43)cc2)cc1] 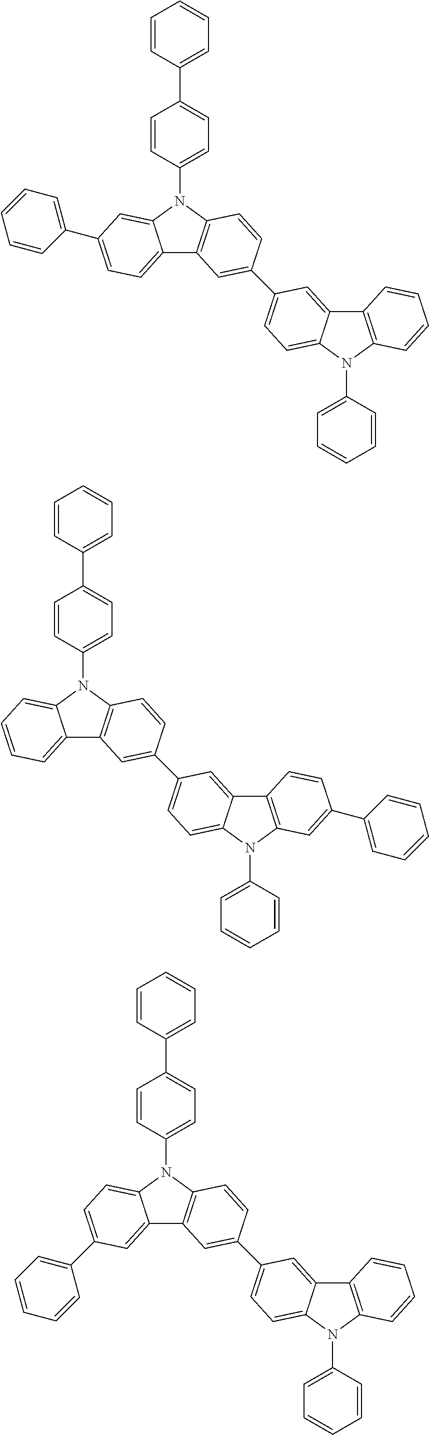 [SMILES: c1ccc(-c2ccc(-n3c4ccc(-c5ccc6c(c5)c5ccccc5n6-c5ccccc5)cc4c4ccc(-c5ccccc5)cc43)cc2)cc1 ; c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1] [chemical structure]
